(19) United States
(12) Patent Application Publication
(10) Pub. No.: US 2026/0247856 A1
(43) Pub. Date: Aug. 20, 2026

(54) COMPOUND FOR ORGANIC OPTOELECTRONIC DEVICE, ORGANIC OPTOELECTRONIC DEVICE AND DISPLAY DEVICE

(71) Applicant: SAMSUNG SDI CO., LTD., Yongin-si, Gyeonggi-do (KR)

(72) Inventors: Jiyun KWON, Suwon-si, Gyeonggi-do (KR); Dong Min KANG, Suwon-si, Gyeonggi-do (KR); Kipo JANG, Suwon-si, Gyeonggi-do (KR); Jonghoon KIM, Suwon-si, Gyeonggi-do (KR); Changwoo KIM, Suwon-si, Gyeonggi-do (KR); Hansol SEO, Suwon-si, Gyeonggi-do (KR); Jongwoo CHOI, Suwon-si, Gyeonggi-do (KR); Hyung Sun KIM, Suwon-si, Gyeonggi-do (KR); Sung-Hyun JUNG, Suwon-si, Gyeonggi-do (KR); Ho Kuk JUNG, Suwon-si, Gyeonggi-do (KR); Youngkyoung JO, Suwon-si, Gyeonggi-do (KR)

(21) Appl. No.: 19/156,561

(22) PCT Filed: Mar. 14, 2024
(86) PCT No.: PCT/KR2024/095503

§ 371 Date: Aug. 14, 2025

(30) Foreign Application Priority Data
Mar. 14, 2023 (KR) 10-2023-0033350

(51) Int. Cl. H10K 85/60; C07B 59/00; C07D 495/04; C07D 519/00; C09K 11/02; H10K 50/11

(52) U.S. Cl. CPC H10K 85/654; C07B 59/002; C07D 495/04; C07D 519/00; C09K 11/02; H10K 85/615; H10K 85/622; H10K 85/657; H10K 85/6572; H10K 85/6574; H10K 50/11

(57) ABSTRACT

Disclosed are a compound for an organic optoelectronic device represented by a combination of Chemical Formula 1 and Chemical Formula 2, an organic optoelectronic device including the same, and a display device.

### TECHNICAL FIELD

A compound for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a compound for an organic optoelectronic device capable of implementing a high-efficiency and long life-span organic optoelectronic device.

Another embodiment provides an organic optoelectronic device including the compound for the organic optoelectronic device.

Another embodiment provides a display device including the organic optoelectronic device.

### Technical Solution

According to an embodiment, a compound for an organic optoelectronic device represented by a combination of a combination of Chemical Formula 1 and Chemical Formula 2.

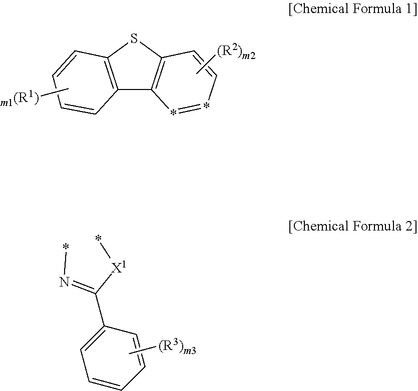

In Chemical Formula 1 and Chemical Formula 2,
X¹ is O or S,
*s are each linking carbon (C),
*in Chemical Formula 1 is linked to * in Chemical Formula 2, respectively,
R¹ and R² are each independently hydrogen, deuterium, a substituted or unsubstituted C1 to C30 alkyl group, a substituted or unsubstituted C6 to C30 aryl group, or a substituted or unsubstituted C2 to C30 heterocyclic group,
R³ is hydrogen, deuterium, a substituted or unsubstituted phenyl group, or a group represented by Chemical Formula a,
m1 is one of integers of 1 to 4,
m2 is an integer of 1 or 2,
m3 is one of integers of 1 to 5, and
at least one of R¹ to R³ is a group represented by Chemical Formula a,

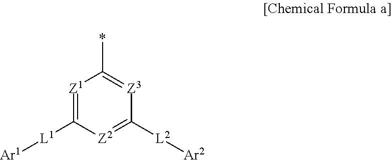

wherein, in Chemical Formula a,
Z¹ to Z³ are each independently N or C-L^{a}-R^{a},
at least two of Z¹ to Z³ are N,
L^{a}, L¹, and L² are each independently a single bond, a substituted or unsubstituted C6 to C30 arylene group, or a substituted or unsubstituted C2 to C30 heterocyclic group,
R^{a} is hydrogen, deuterium, a substituted or unsubstituted C1 to C30 alkyl group, a substituted or unsubstituted C6 to C30 aryl group, or a substituted or unsubstituted C2 to C30 heterocyclic group, and
Ar¹ and Ar² are each independently a substituted or unsubstituted C6 to C30 aryl group or a substituted or unsubstituted C2 to C30 heterocyclic group.

According to another embodiment, an organic optoelectronic device includes an anode and a cathode facing each other, and at least one organic layer between the anode and the cathode, wherein the organic layer includes the compound for the organic optoelectronic device.

According to another embodiment, a display device including the organic optoelectronic device is provided.

### Advantageous Effects

An organic optoelectronic device having high efficiency and a long life-span may be realized.

### DESCRIPTION OF THE DRAWINGS

FIG. 1 is a cross-sectional view illustrating an organic light emitting diode according to an embodiment.

### DESCRIPTION OF SYMBOLS

100: organic light emitting diode
105: organic layer
110: cathode
120: anode
130: light emitting layer
140: hole transport region
150: electron transport region

### BEST MODE

Hereinafter, embodiments of the present invention are described in detail. However, these embodiments are exemplary, and this disclosure is not limited thereto.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present invention, the “substituted” refers to replacement of at least one hydrogen of a substituent or a compound by deuterium, a C1 to C20 alkyl group, a C6 to C30 aryl group, or a cyano group. In specific example of the present invention, the “substituted” refers to replacement of at least one hydrogen of a substituent or a compound by deuterium, a C1 to C5 alkyl group, a C6 to C18 aryl group, or a cyano group. In specific example of the present invention, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

In the present specification, “unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).”

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

As an example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzooxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof, but is not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electronic characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

The compound for the organic optoelectronic device according to an embodiment is represented by a combination of Chemical Formula 1 and Chemical Formula 2.

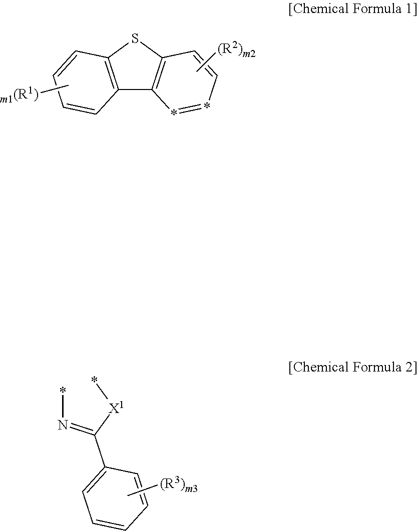

In Chemical Formula 1 and Chemical Formula 2,
X¹ is O or S,
*s are each linking carbon (C),
* in Chemical Formula 1 is linked to * in Chemical Formula 2, respectively,
R¹ and R² are each independently hydrogen, deuterium, a substituted or unsubstituted C1 to C30 alkyl group, a substituted or unsubstituted C6 to C30 aryl group, or a substituted or unsubstituted C2 to C30 heterocyclic group,
R³ is hydrogen, deuterium, a substituted or unsubstituted phenyl group, or a group represented by Chemical Formula a,
m1 is one of integers of 1 to 4,
m2 is an integer of 1 or 2,
m3 is one of integers of 1 to 5, and
at least one of R¹ to R³ is a group represented by Chemical Formula a,

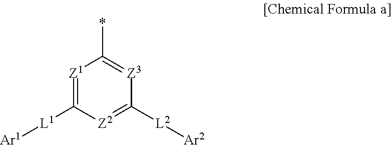

wherein, in Chemical Formula a,
Z¹ to Z³ are each independently N or C-L^{a}-R^{a},
at least two of Z¹ to Z³ are N,
L^{a}, L¹, and L² are each independently a single bond, a substituted or unsubstituted C6 to C30 arylene group, or a substituted or unsubstituted C2 to C30 heterocyclic group,
R^{a} is hydrogen, deuterium, a substituted or unsubstituted C1 to C30 alkyl group, a substituted or unsubstituted C6 to C30 aryl group, or a substituted or unsubstituted C2 to C30 heterocyclic group, and
Ar¹ and Ar² are each independently a substituted or unsubstituted C6 to C30 aryl group or a substituted or unsubstituted C2 to C30 heterocyclic group.

The first compound has the advantages of high charge mobility, including an azolo-dibenzothiophene core, a large current on/off ratio, very low driving voltage, and excellent efficiency and life-span characteristics.

In addition, the benzoxazole-derived moiety is structurally characterized by low dielectric constant and has a low dipole moment value, which enables excellent image quality implementation when applied to final devices.

In particular, the hole mobility of the structure fused at 1st and 2nd positions of dibenzothiophene is more excellent, and accordingly, the organic light emitting diode including it has low voltage/high efficiency/long life-span characteristics. Meanwhile, the carbon between the two heteroatoms, nitrogen and oxygen (or sulfur), of oxazole (or thiophene) has a weak bonding force and is a vulnerable moiety that is easily broken or causes side reactions due to heat, etc., so by replacing this moiety with an aromatic ring such as phenyl, the heat resistance and durability of the material can be improved.

In Chemical Formula 1, when m1 is 2 or more, each R¹ may be the same or different from each other.

In Chemical Formula 1, when m2 is 2 or more, each R² may be the same or different from each other.

In Chemical Formula 2, when m3 is 2 or more, each R³ may be the same or different from each other.

The combination of Chemical Formula 1 and Chemical Formula 2 may be represented by, for example, Chemical Formula 1A or Chemical Formula 1B, depending on the fusion position and fusion direction.

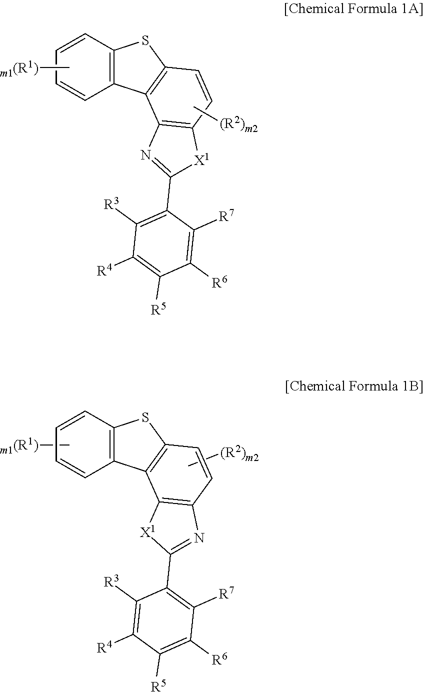

In Chemical Formula 1A to Chemical Formula 1B,
X¹, R¹ to R³, and m1 to m3 are defined as described above.

A specific example of the combination of Chemical Formula 1 and Chemical Formula 2 may be represented by any one of Chemical Formula 1A-I, Chemical Formula 1A-II, Chemical Formula 1B-I, Chemical Formula 1B-II, Chemical Formula 1C-I, and Chemical Formula 1C-II.

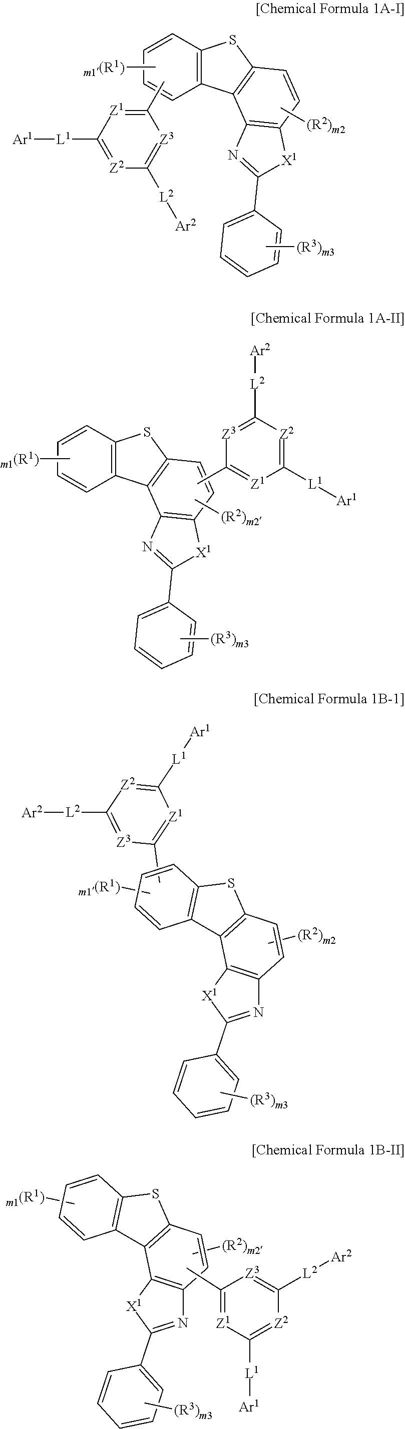

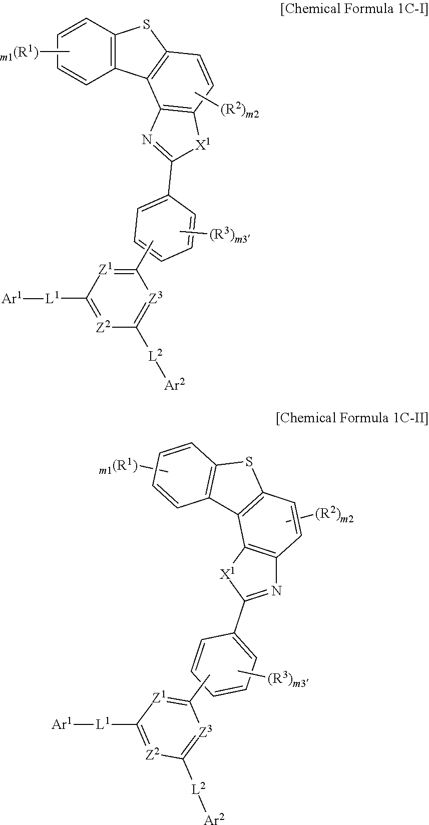

In Chemical Formula 1A-I, Chemical Formula 1A-II, Chemical Formula 1B-I, Chemical Formula 1B-II, Chemical Formula 1C-I, and Chemical Formula 1C-II,
X¹, Z¹ to Z³, Ar¹, Ar², L¹, L², and m1 to m3 are defined as described above,
R¹ and R² are each independently hydrogen, deuterium, a substituted or unsubstituted C1 to C30 alkyl group, a substituted or unsubstituted C6 to C30 aryl group, or a substituted or unsubstituted C2 to C30 heterocyclic group,
R³ is hydrogen, deuterium, or a substituted or unsubstituted phenyl group,
m1′ is one of integers of 1 to 3,
m2′ is 1, and
m3′ is one of integers of 1 to 4.

A specific example of the combination of Chemical Formula 1 and Chemical Formula 2 may be represented by any one of Chemical Formula 1A-I, Chemical Formula 1A-II, Chemical Formula 1B-I, and Chemical Formula 1B-II.

A more specific example of the combination of Chemical Formula 1 and Chemical Formula 2 may be represented by any one of Chemical Formula 1A-I-1 to Chemical Formula 1A-I-4, Chemical Formula 1A-II-1, Chemical Formula 1A-II-2, Chemical Formula 1B-I-1 to Chemical Formula 1B-I-4, Chemical Formula 1B-II-1, and Chemical Formula 1B-II-2.

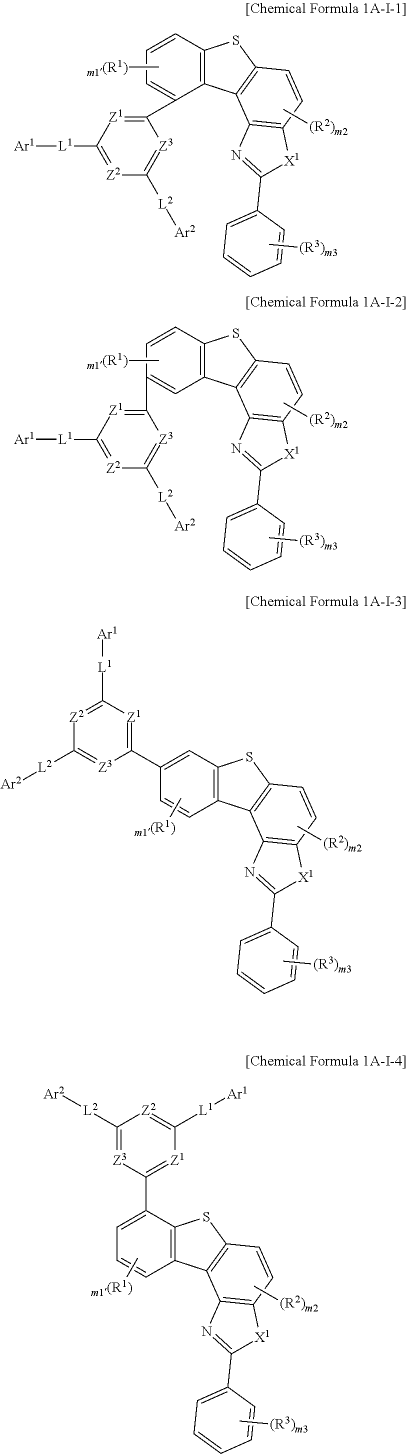

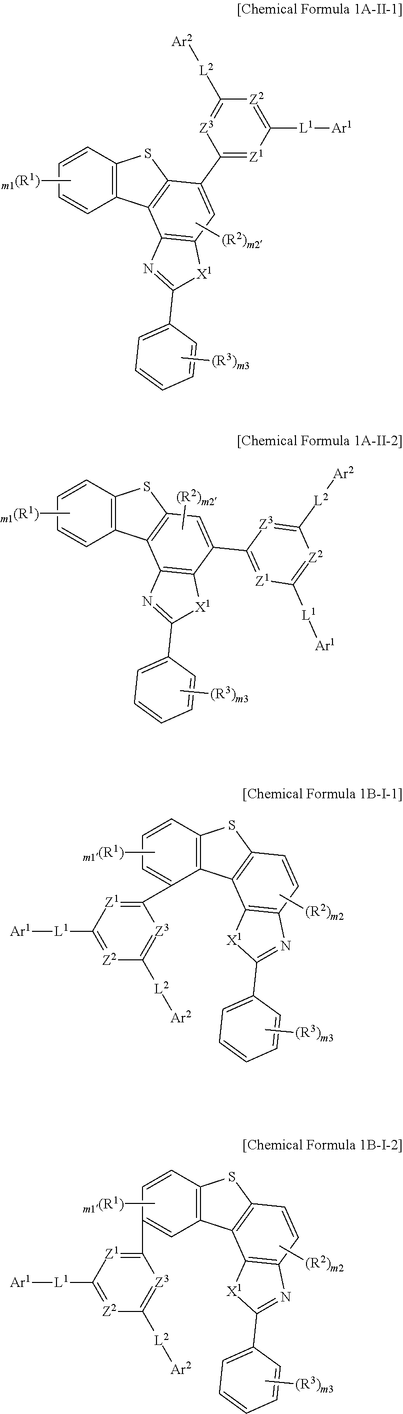

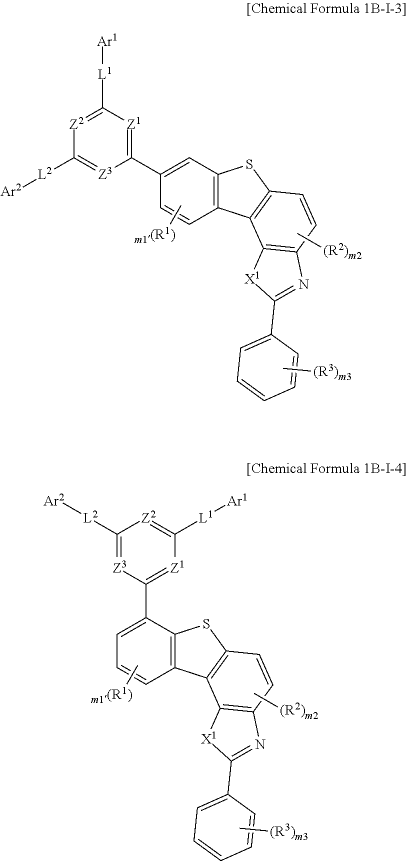

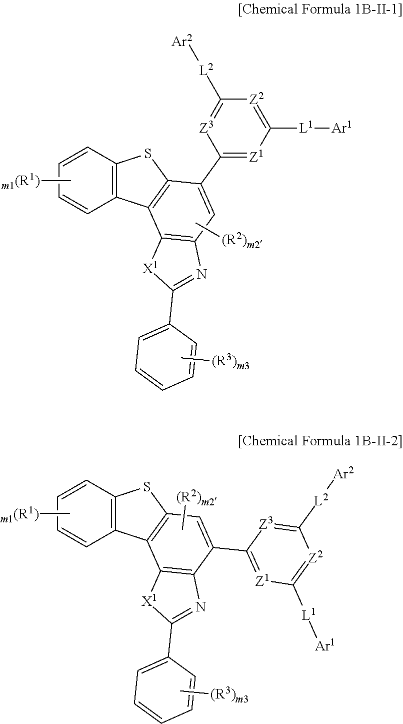

In Chemical Formula 1A-I-1 to Chemical Formula 1A-I-4, Chemical Formula 1A-II-1, Chemical Formula 1A-II-2, Chemical Formula 1B-I-1 to Chemical Formula 1B-I-4, Chemical Formula 1B-II-1, and Chemical Formula 1B-II-2,
X¹, Z¹ to Z³, R¹ to R³, Ar¹, Ar², L¹, L², m1, m1′, m2, m2′, and m3 are as described above.

A more specific example of the combination of Chemical Formula 1 and Chemical Formula 2 may be represented by any one of Chemical Formula 1A-I-1 to Chemical Formula 1A-I-4, Chemical Formula 1A-II-1, Chemical Formula 1B-I-1 to Chemical Formula 1B-I-4, and Chemical Formula 1B-II-1.

For example, R¹ and R² may each independently be hydrogen, deuterium, a substituted or unsubstituted C1 to C10 alkyl group, a substituted or unsubstituted C6 to C12 aryl group, or a substituted or unsubstituted C2 to C20 heterocyclic group.

As a specific example, R¹ and R² may each independently be hydrogen, deuterium, a substituted or unsubstituted C1 to C5 alkyl group, a substituted or unsubstituted phenyl group, a substituted or unsubstituted biphenyl group, or a substituted or unsubstituted naphthyl group.

For example, L^{a}, L¹, and L² may each independently be a single bond, a substituted or unsubstituted phenylene group, or a substituted or unsubstituted biphenylene group.

For example, Ar¹ and Ar² may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, a substituted or unsubstituted dibenzosilolyl group, or a substituted or unsubstituted indolocarbazolyl group.

For example, L¹-Ar¹ and L²-Ar² may each independently be selected from the substituents listed in Group I.

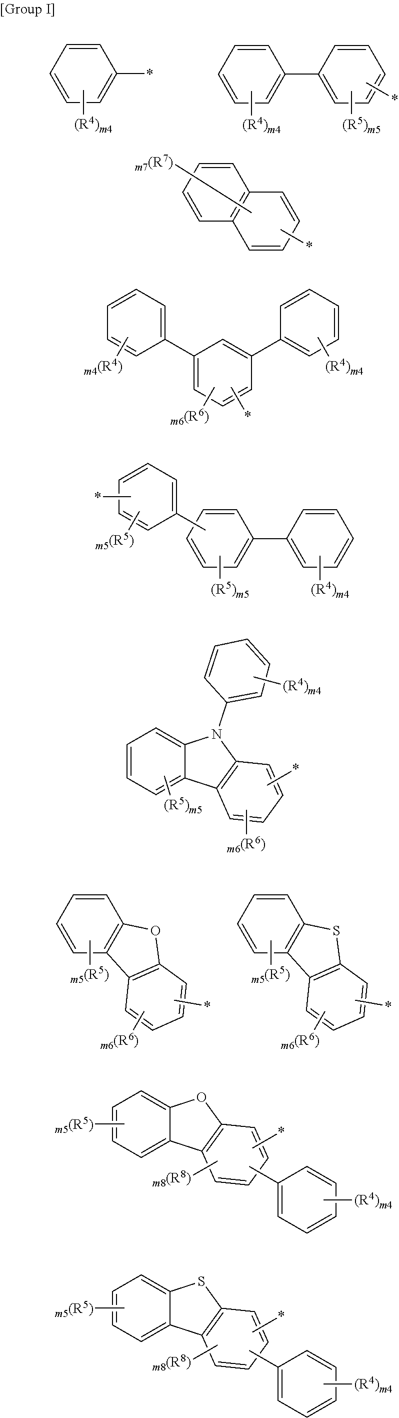

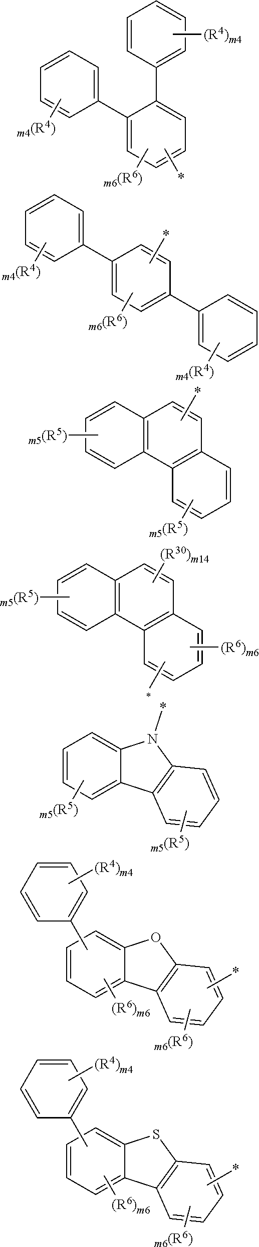

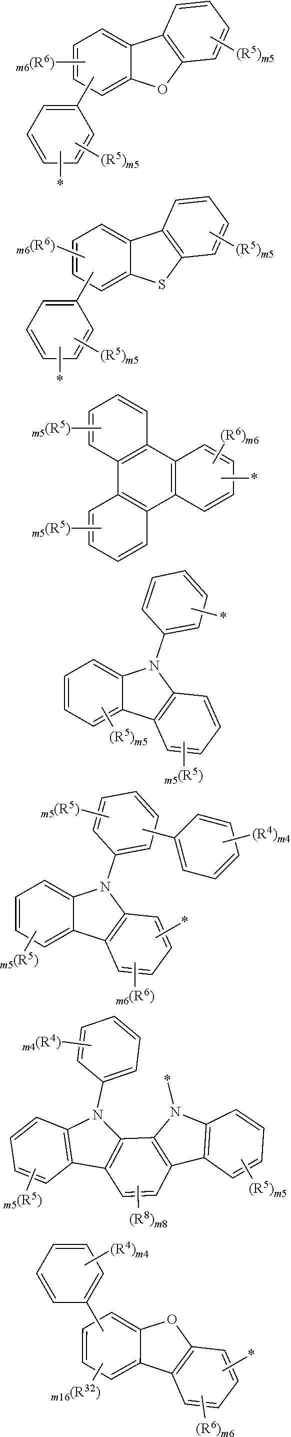

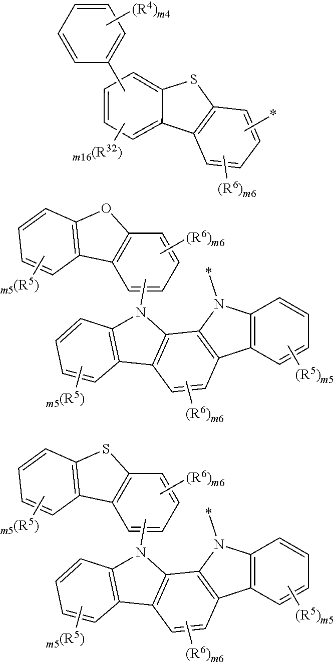

In Group I,
R⁴ to R⁷ are each independently hydrogen, deuterium, a cyano group, a C1 to C10 alkyl group, or a C6 to C12 aryl group,
m4 is one of integers of 1 to 5,
m5 is one of integers of 1 to 4,
m6 is one of integers of 1 to 3,
m7 is one of integers of 1 to 7,
m8 is an integer of 1 or 2, and
* is a linking point.

In Group I, when m4 is 2 or more, each R⁴ may be the same or different from each other.

In Group I, when m5 is 2 or more, each R⁵ may be the same or different from each other.

In Group I, when m6 is 2 or more, each R⁶ may be the same or different from each other.

In Group I, when m7 is 2 or more, each R⁷ may be the same or different from each other.

In Group I, when m8 is 2 is 2 or more, each R⁸ may be the same or different from each other.

In the most specific embodiment, the specific examples of the combination of Chemical Formula 1 and Chemical Formula 2 may be one selected from the compounds listed in Group 1, but are not limited thereto.

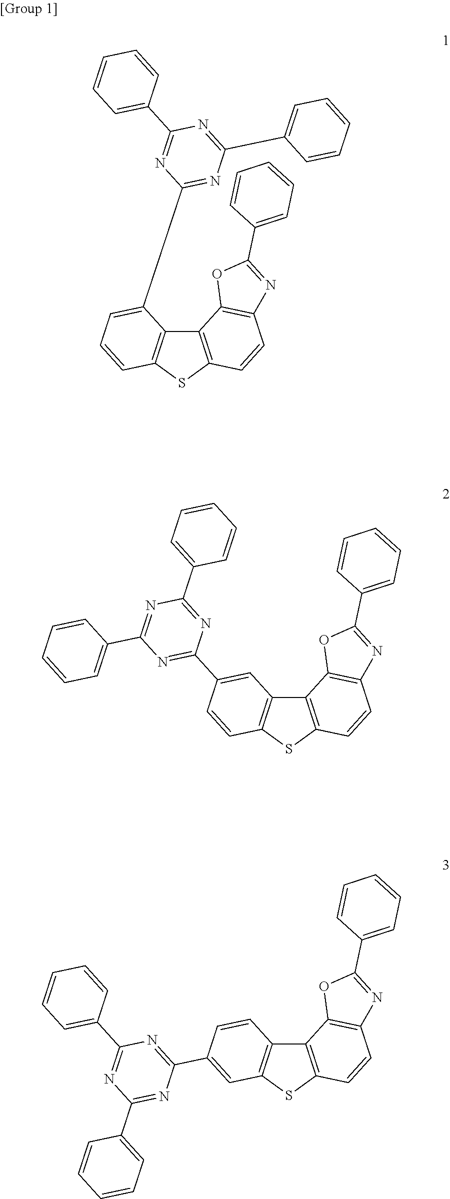 [SMILES: c1ccc(-c2nc(-c3ccccc3)nc(-c3cccc4sc5ccc6nc(-c7ccccc7)oc6c5c34)n2)cc1 ; c1ccc(-c2nc(-c3ccccc3)nc(-c3ccc4sc5ccc6nc(-c7ccccc7)oc6c5c4c3)n2)cc1 ; c1ccc(-c2nc(-c3ccccc3)nc(-c3ccc4c(c3)sc3ccc5nc(-c6ccccc6)oc5c34)n2)cc1]

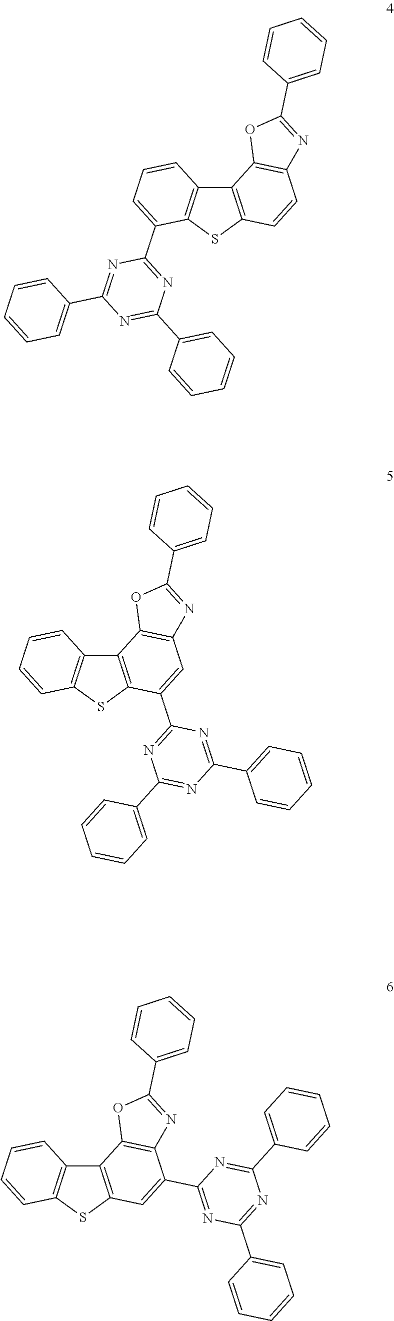 [SMILES: c1ccc(-c2nc(-c3ccccc3)nc(-c3cccc4c3sc3ccc5nc(-c6ccccc6)oc5c34)n2)cc1 ; c1ccc(-c2nc(-c3ccccc3)nc(-c3cc4nc(-c5ccccc5)oc4c4c3sc3ccccc34)n2)cc1 ; c1ccc(-c2nc(-c3ccccc3)nc(-c3cc4sc5ccccc5c4c4oc(-c5ccccc5)nc34)n2)cc1]

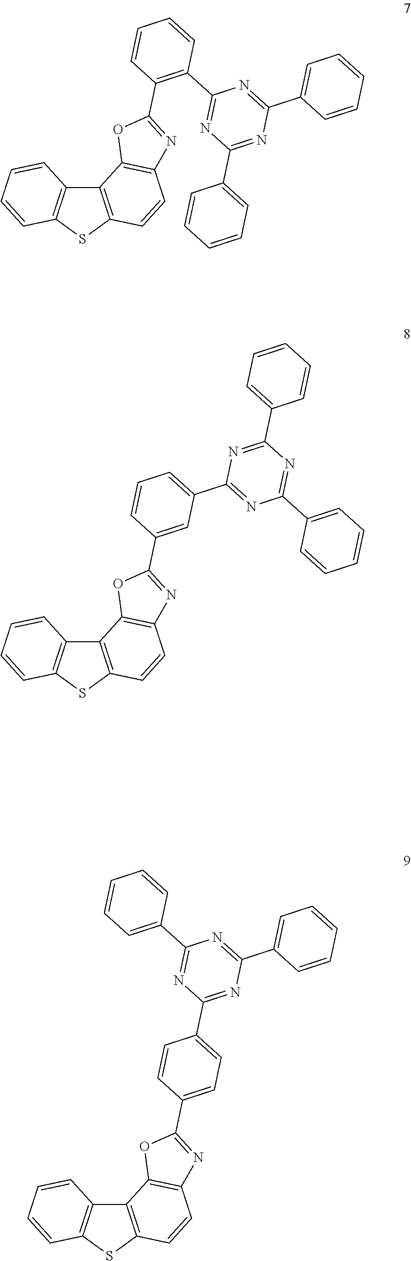 [SMILES: c1ccc(-c2nc(-c3ccccc3)nc(-c3ccccc3-c3nc4ccc5sc6ccccc6c5c4o3)n2)cc1 ; c1ccc(-c2nc(-c3ccccc3)nc(-c3cccc(-c4nc5ccc6sc7ccccc7c6c5o4)c3)n2)cc1 ; c1ccc(-c2nc(-c3ccccc3)nc(-c3ccc(-c4nc5ccc6sc7ccccc7c6c5o4)cc3)n2)cc1]

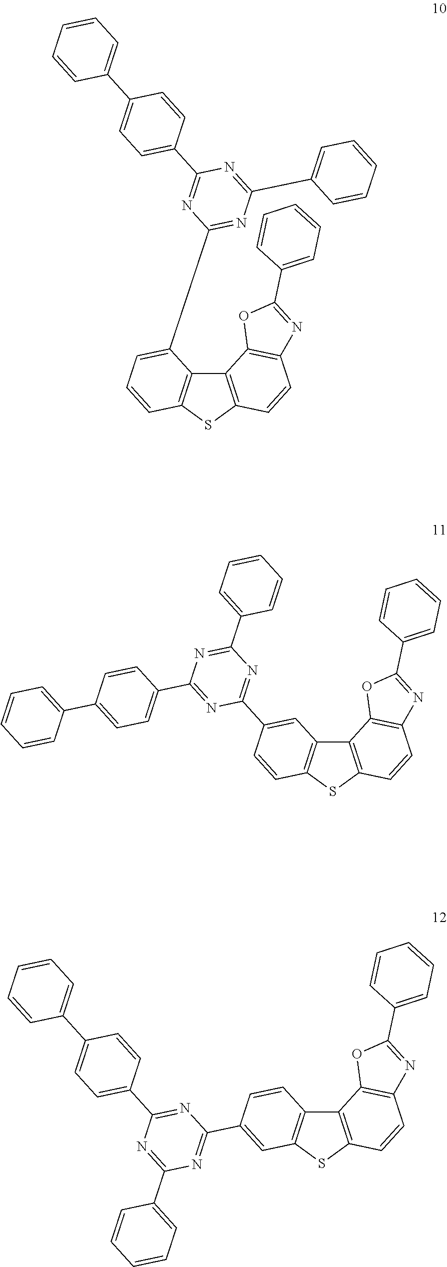 [SMILES: c1ccc(-c2ccc(-c3nc(-c4ccccc4)nc(-c4cccc5sc6ccc7nc(-c8ccccc8)oc7c6c45)n3)cc2)cc1 ; c1ccc(-c2ccc(-c3nc(-c4ccccc4)nc(-c4ccc5sc6ccc7nc(-c8ccccc8)oc7c6c5c4)n3)cc2)cc1 ; c1ccc(-c2ccc(-c3nc(-c4ccccc4)nc(-c4ccc5c(c4)sc4ccc6nc(-c7ccccc7)oc6c45)n3)cc2)cc1]

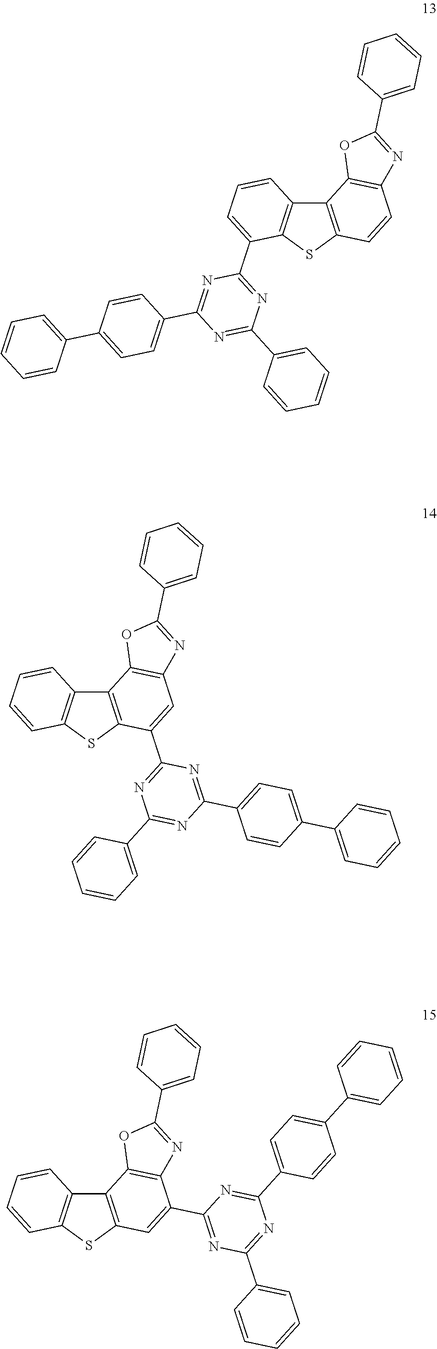 [SMILES: c1ccc(-c2ccc(-c3nc(-c4ccccc4)nc(-c4cccc5c4sc4ccc6nc(-c7ccccc7)oc6c45)n3)cc2)cc1 ; c1ccc(-c2ccc(-c3nc(-c4ccccc4)nc(-c4cc5nc(-c6ccccc6)oc5c5c4sc4ccccc45)n3)cc2)cc1 ; c1ccc(-c2ccc(-c3nc(-c4ccccc4)nc(-c4cc5sc6ccccc6c5c5oc(-c6ccccc6)nc45)n3)cc2)cc1]

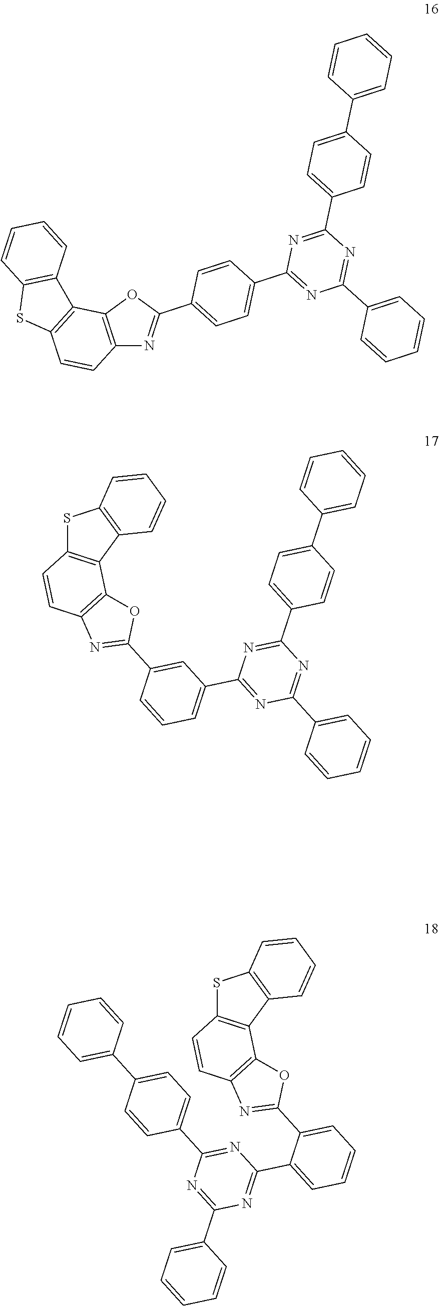 [SMILES: c1ccc(-c2ccc(-c3nc(-c4ccccc4)nc(-c4ccc(-c5nc6ccc7sc8ccccc8c7c6o5)cc4)n3)cc2)cc1 ; c1ccc(-c2ccc(-c3nc(-c4ccccc4)nc(-c4cccc(-c5nc6ccc7sc8ccccc8c7c6o5)c4)n3)cc2)cc1 ; c1ccc(-c2ccc(-c3nc(-c4ccccc4)nc(-c4ccccc4-c4nc5ccc6sc7ccccc7c6c5o4)n3)cc2)cc1]

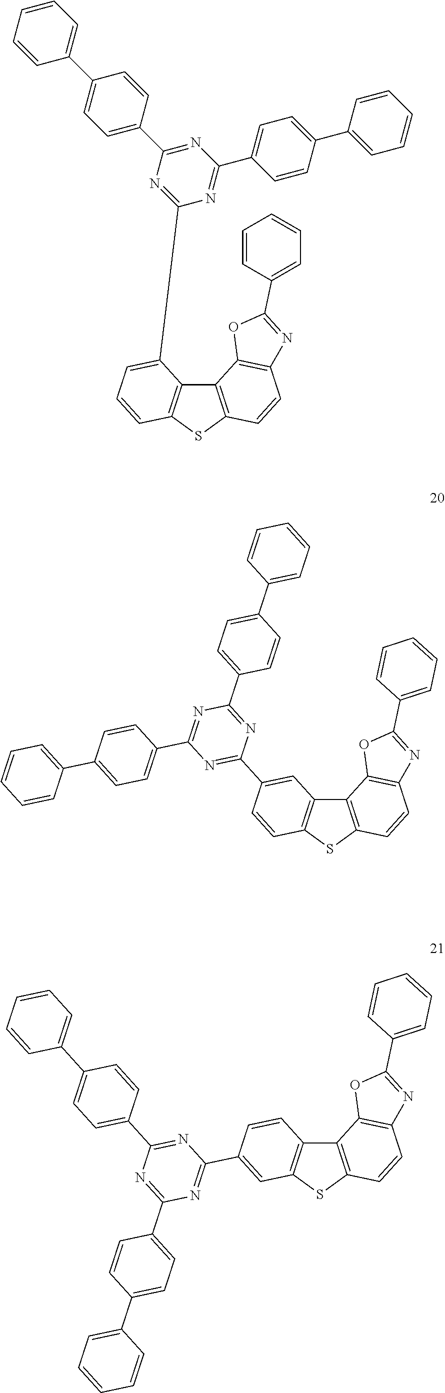 [SMILES: c1ccc(-c2ccc(-c3nc(-c4ccc(-c5ccccc5)cc4)nc(-c4cccc5sc6ccc7nc(-c8ccccc8)oc7c6c45)n3)cc2)cc1 ; c1ccc(-c2ccc(-c3nc(-c4ccc(-c5ccccc5)cc4)nc(-c4ccc5sc6ccc7nc(-c8ccccc8)oc7c6c5c4)n3)cc2)cc1 ; c1ccc(-c2ccc(-c3nc(-c4ccc(-c5ccccc5)cc4)nc(-c4ccc5c(c4)sc4ccc6nc(-c7ccccc7)oc6c45)n3)cc2)cc1]

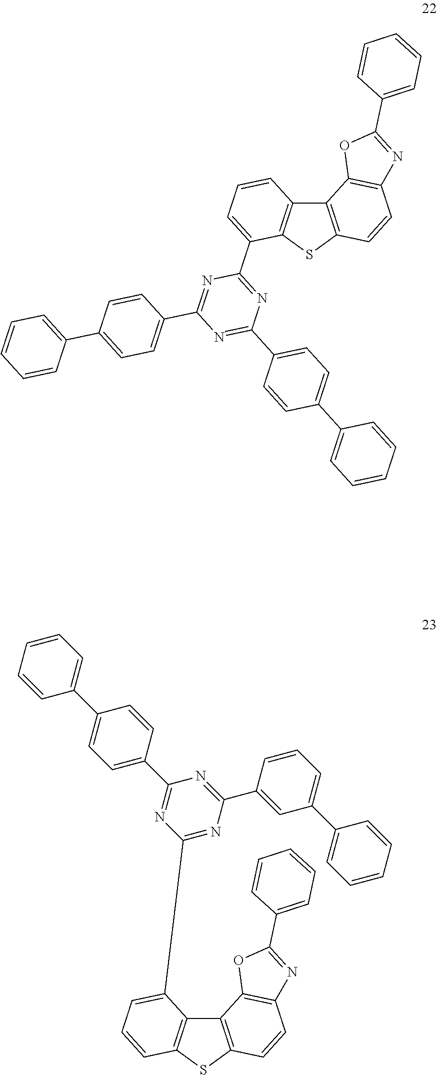 [SMILES: c1ccc(-c2ccc(-c3nc(-c4ccc(-c5ccccc5)cc4)nc(-c4cccc5c4sc4ccc6nc(-c7ccccc7)oc6c45)n3)cc2)cc1 ; c1ccc(-c2ccc(-c3nc(-c4cccc(-c5ccccc5)c4)nc(-c4cccc5sc6ccc7nc(-c8ccccc8)oc7c6c45)n3)cc2)cc1]

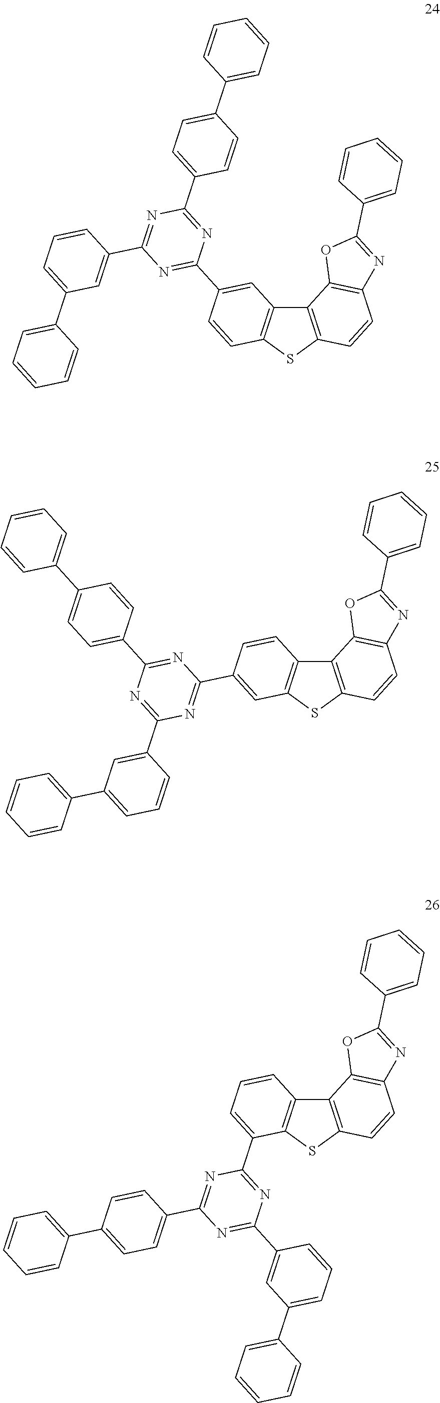 [SMILES: c1ccc(-c2ccc(-c3nc(-c4cccc(-c5ccccc5)c4)nc(-c4ccc5sc6ccc7nc(-c8ccccc8)oc7c6c5c4)n3)cc2)cc1 ; c1ccc(-c2ccc(-c3nc(-c4cccc(-c5ccccc5)c4)nc(-c4ccc5c(c4)sc4ccc6nc(-c7ccccc7)oc6c45)n3)cc2)cc1 ; c1ccc(-c2ccc(-c3nc(-c4cccc(-c5ccccc5)c4)nc(-c4cccc5c4sc4ccc6nc(-c7ccccc7)oc6c45)n3)cc2)cc1]

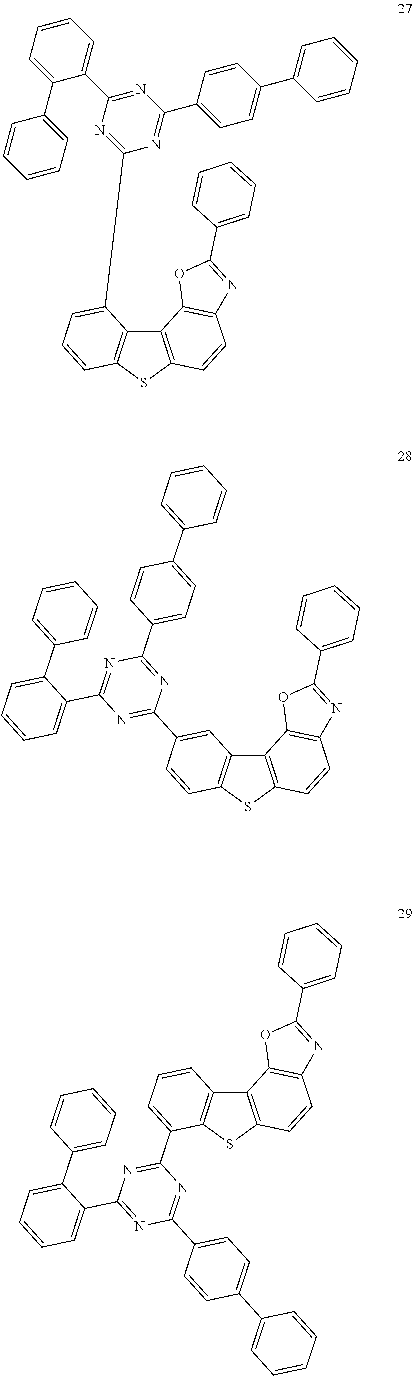 [SMILES: c1ccc(-c2ccc(-c3nc(-c4ccccc4-c4ccccc4)nc(-c4cccc5sc6ccc7nc(-c8ccccc8)oc7c6c45)n3)cc2)cc1 ; c1ccc(-c2ccc(-c3nc(-c4ccc5sc6ccc7nc(-c8ccccc8)oc7c6c5c4)nc(-c4ccccc4-c4ccccc4)n3)cc2)cc1 ; c1ccc(-c2ccc(-c3nc(-c4ccccc4-c4ccccc4)nc(-c4cccc5c4sc4ccc6nc(-c7ccccc7)oc6c45)n3)cc2)cc1]

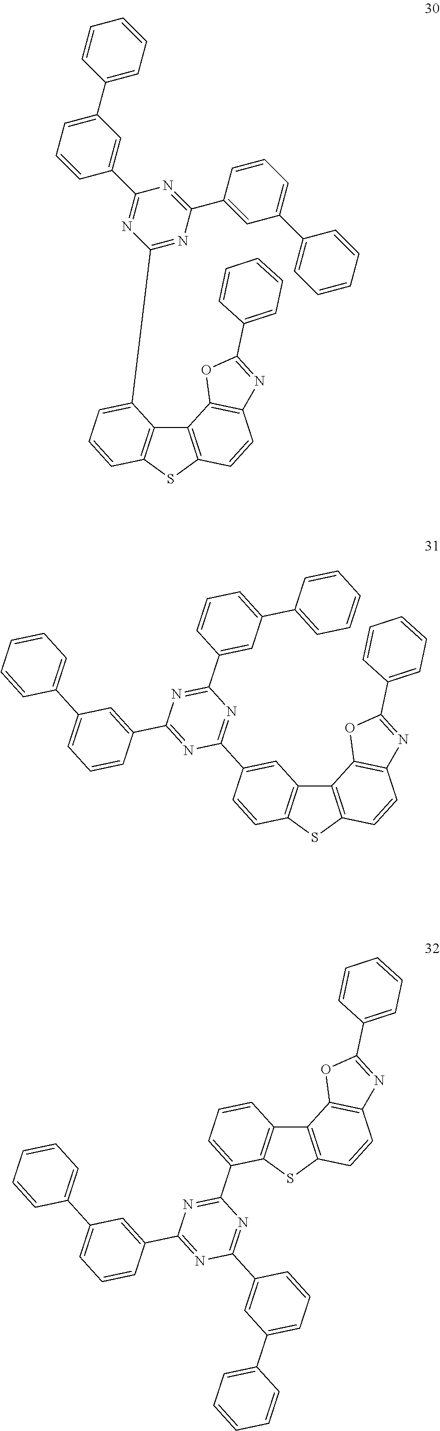 [SMILES: c1ccc(-c2cccc(-c3nc(-c4cccc(-c5ccccc5)c4)nc(-c4cccc5sc6ccc7nc(-c8ccccc8)oc7c6c45)n3)c2)cc1 ; c1ccc(-c2cccc(-c3nc(-c4cccc(-c5ccccc5)c4)nc(-c4ccc5sc6ccc7nc(-c8ccccc8)oc7c6c5c4)n3)c2)cc1 ; c1ccc(-c2cccc(-c3nc(-c4cccc(-c5ccccc5)c4)nc(-c4cccc5c4sc4ccc6nc(-c7ccccc7)oc6c45)n3)c2)cc1]

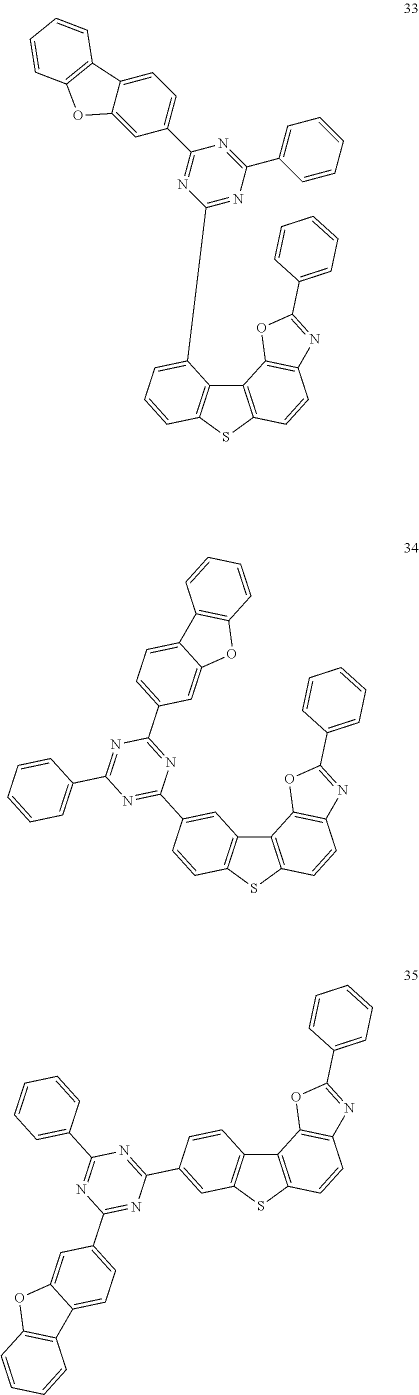 [SMILES: c1ccc(-c2nc(-c3ccc4c(c3)oc3ccccc34)nc(-c3cccc4sc5ccc6nc(-c7ccccc7)oc6c5c34)n2)cc1 ; c1ccc(-c2nc(-c3ccc4c(c3)oc3ccccc34)nc(-c3ccc4sc5ccc6nc(-c7ccccc7)oc6c5c4c3)n2)cc1 ; c1ccc(-c2nc(-c3ccc4c(c3)oc3ccccc34)nc(-c3ccc4c(c3)sc3ccc5nc(-c6ccccc6)oc5c34)n2)cc1]

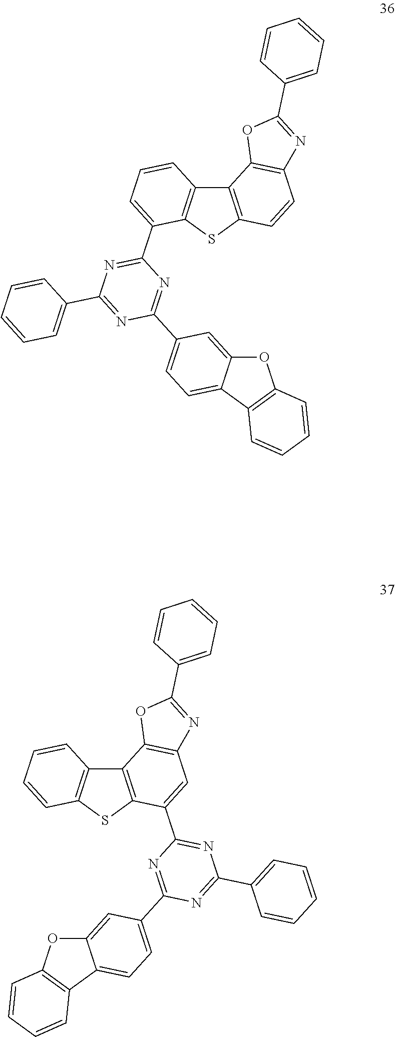 [SMILES: c1ccc(-c2nc(-c3ccc4c(c3)oc3ccccc34)nc(-c3cccc4c3sc3ccc5nc(-c6ccccc6)oc5c34)n2)cc1 ; c1ccc(-c2nc(-c3ccc4c(c3)oc3ccccc34)nc(-c3cc4nc(-c5ccccc5)oc4c4c3sc3ccccc34)n2)cc1]

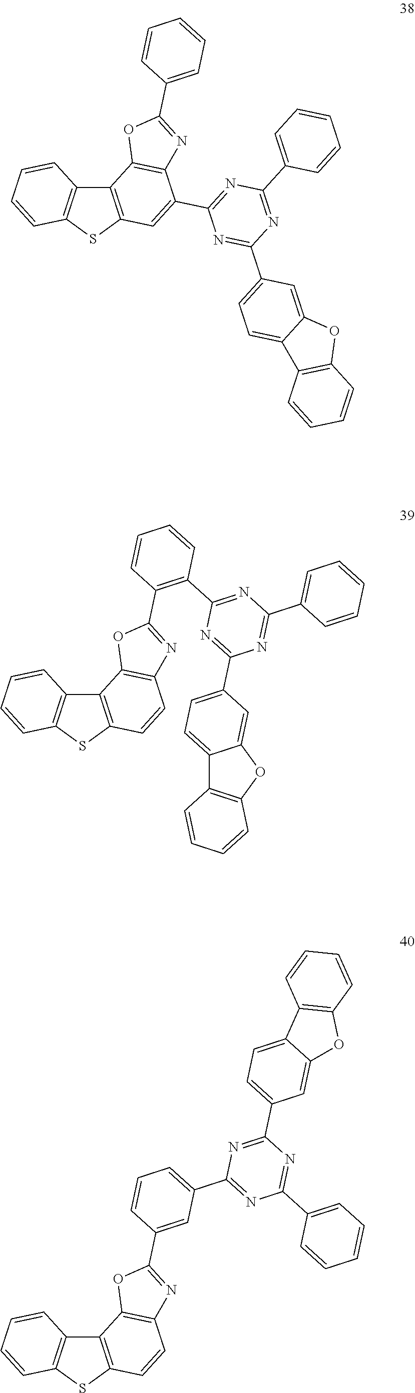 [SMILES: c1ccc(-c2nc(-c3ccc4c(c3)oc3ccccc34)nc(-c3cc4sc5ccccc5c4c4oc(-c5ccccc5)nc34)n2)cc1 ; c1ccc(-c2nc(-c3ccc4c(c3)oc3ccccc34)nc(-c3ccccc3-c3nc4ccc5sc6ccccc6c5c4o3)n2)cc1 ; c1ccc(-c2nc(-c3cccc(-c4nc5ccc6sc7ccccc7c6c5o4)c3)nc(-c3ccc4c(c3)oc3ccccc34)n2)cc1]

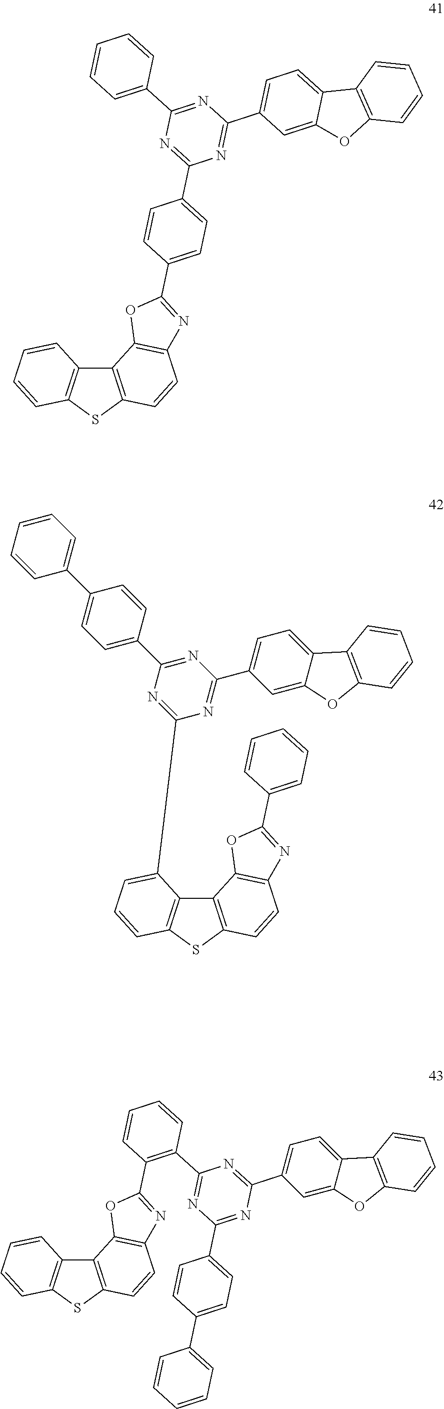 [SMILES: c1ccc(-c2nc(-c3ccc(-c4nc5ccc6sc7ccccc7c6c5o4)cc3)nc(-c3ccc4c(c3)oc3ccccc34)n2)cc1 ; c1ccc(-c2ccc(-c3nc(-c4ccc5c(c4)oc4ccccc45)nc(-c4cccc5sc6ccc7nc(-c8ccccc8)oc7c6c45)n3)cc2)cc1 ; c1ccc(-c2ccc(-c3nc(-c4ccc5c(c4)oc4ccccc45)nc(-c4ccccc4-c4nc5ccc6sc7ccccc7c6c5o4)n3)cc2)cc1]

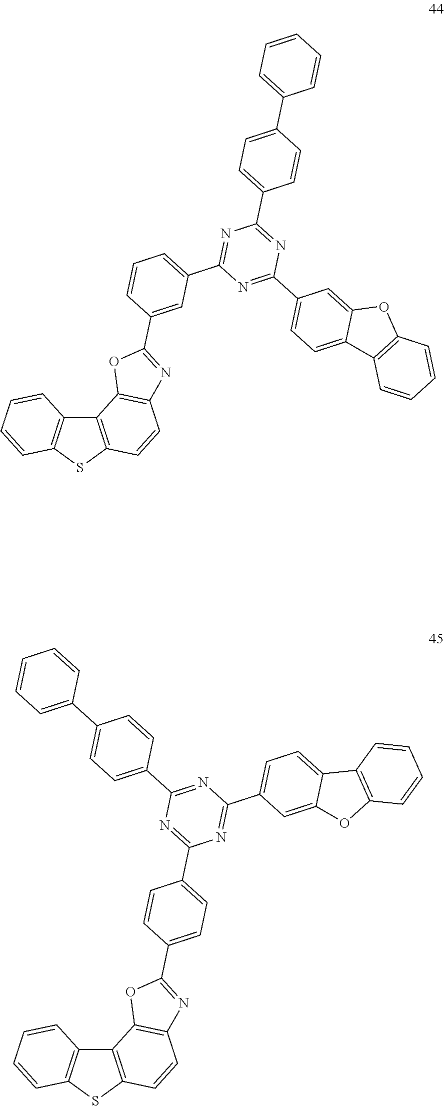 [SMILES: c1ccc(-c2ccc(-c3nc(-c4cccc(-c5nc6ccc7sc8ccccc8c7c6o5)c4)nc(-c4ccc5c(c4)oc4ccccc45)n3)cc2)cc1 ; c1ccc(-c2ccc(-c3nc(-c4ccc(-c5nc6ccc7sc8ccccc8c7c6o5)cc4)nc(-c4ccc5c(c4)oc4ccccc45)n3)cc2)cc1]

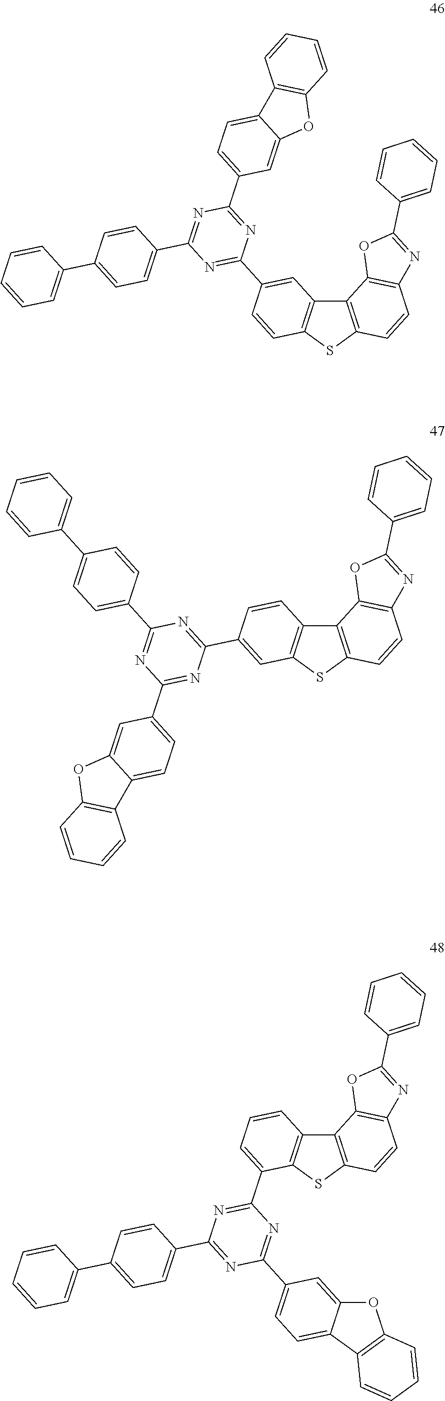 [SMILES: c1ccc(-c2ccc(-c3nc(-c4ccc5c(c4)oc4ccccc45)nc(-c4ccc5sc6ccc7nc(-c8ccccc8)oc7c6c5c4)n3)cc2)cc1 ; c1ccc(-c2ccc(-c3nc(-c4ccc5c(c4)oc4ccccc45)nc(-c4ccc5c(c4)sc4ccc6nc(-c7ccccc7)oc6c45)n3)cc2)cc1 ; c1ccc(-c2ccc(-c3nc(-c4ccc5c(c4)oc4ccccc45)nc(-c4cccc5c4sc4ccc6nc(-c7ccccc7)oc6c45)n3)cc2)cc1]

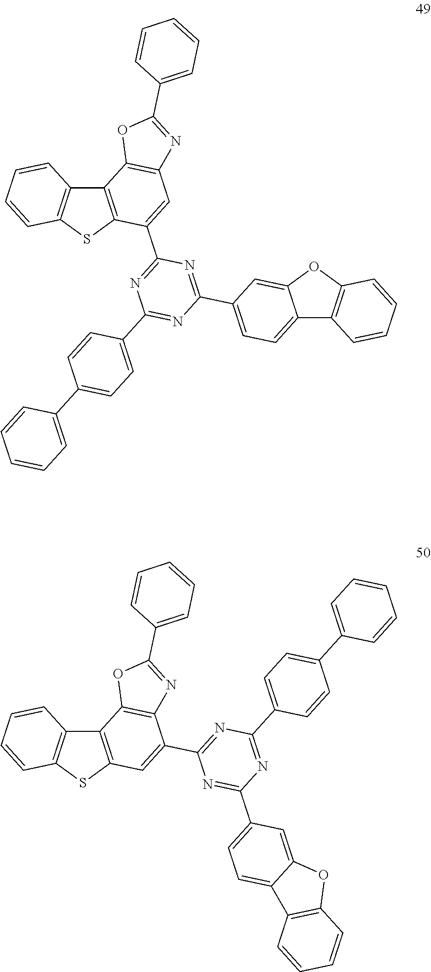 [SMILES: c1ccc(-c2ccc(-c3nc(-c4ccc5c(c4)oc4ccccc45)nc(-c4cc5nc(-c6ccccc6)oc5c5c4sc4ccccc45)n3)cc2)cc1 ; c1ccc(-c2ccc(-c3nc(-c4ccc5c(c4)oc4ccccc45)nc(-c4cc5sc6ccccc6c5c5oc(-c6ccccc6)nc45)n3)cc2)cc1]

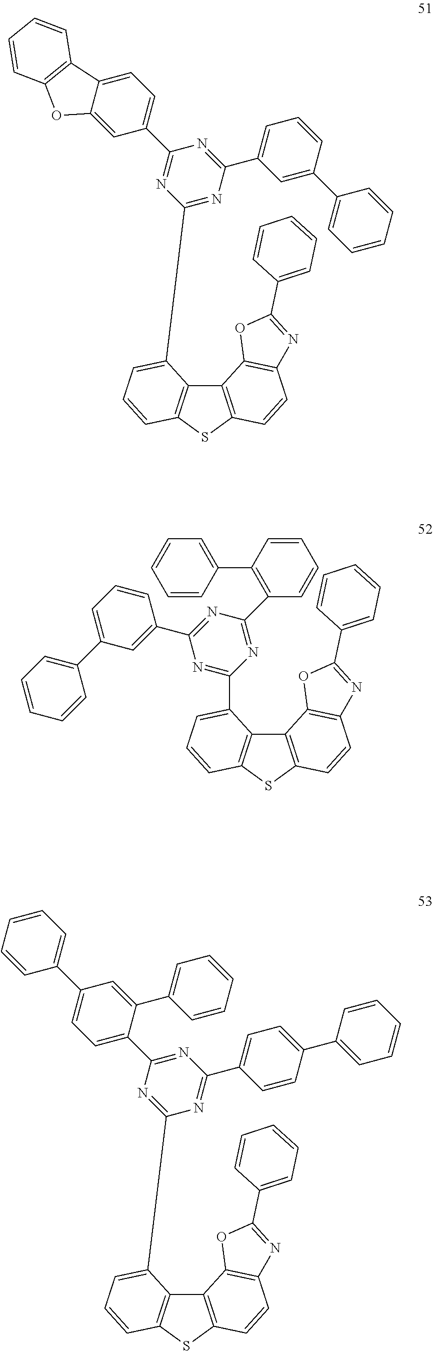 [SMILES: c1ccc(-c2cccc(-c3nc(-c4ccc5c(c4)oc4ccccc45)nc(-c4cccc5sc6ccc7nc(-c8ccccc8)oc7c6c45)n3)c2)cc1 ; c1ccc(-c2cccc(-c3nc(-c4ccccc4-c4ccccc4)nc(-c4cccc5sc6ccc7nc(-c8ccccc8)oc7c6c45)n3)c2)cc1 ; c1ccc(-c2ccc(-c3nc(-c4ccc(-c5ccccc5)cc4-c4ccccc4)nc(-c4cccc5sc6ccc7nc(-c8ccccc8)oc7c6c45)n3)cc2)cc1]

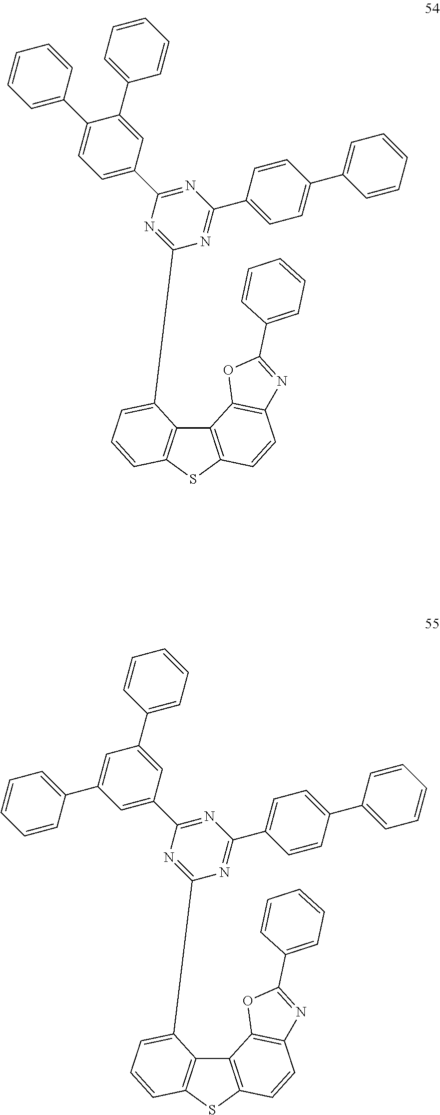 [SMILES: c1ccc(-c2ccc(-c3nc(-c4ccc(-c5ccccc5)c(-c5ccccc5)c4)nc(-c4cccc5sc6ccc7nc(-c8ccccc8)oc7c6c45)n3)cc2)cc1 ; c1ccc(-c2ccc(-c3nc(-c4cc(-c5ccccc5)cc(-c5ccccc5)c4)nc(-c4cccc5sc6ccc7nc(-c8ccccc8)oc7c6c45)n3)cc2)cc1]

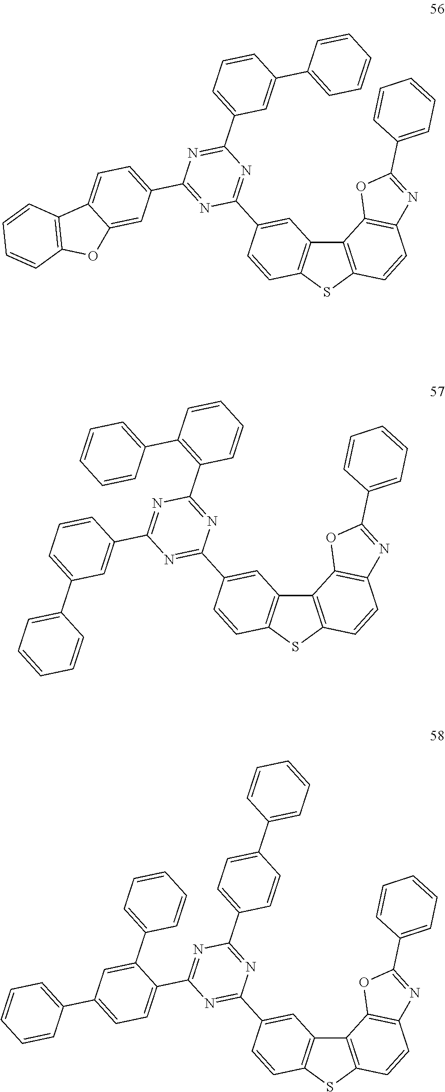 [SMILES: c1ccc(-c2cccc(-c3nc(-c4ccc5c(c4)oc4ccccc45)nc(-c4ccc5sc6ccc7nc(-c8ccccc8)oc7c6c5c4)n3)c2)cc1 ; c1ccc(-c2cccc(-c3nc(-c4ccc5sc6ccc7nc(-c8ccccc8)oc7c6c5c4)nc(-c4ccccc4-c4ccccc4)n3)c2)cc1 ; c1ccc(-c2ccc(-c3nc(-c4ccc5sc6ccc7nc(-c8ccccc8)oc7c6c5c4)nc(-c4ccc(-c5ccccc5)cc4-c4ccccc4)n3)cc2)cc1]

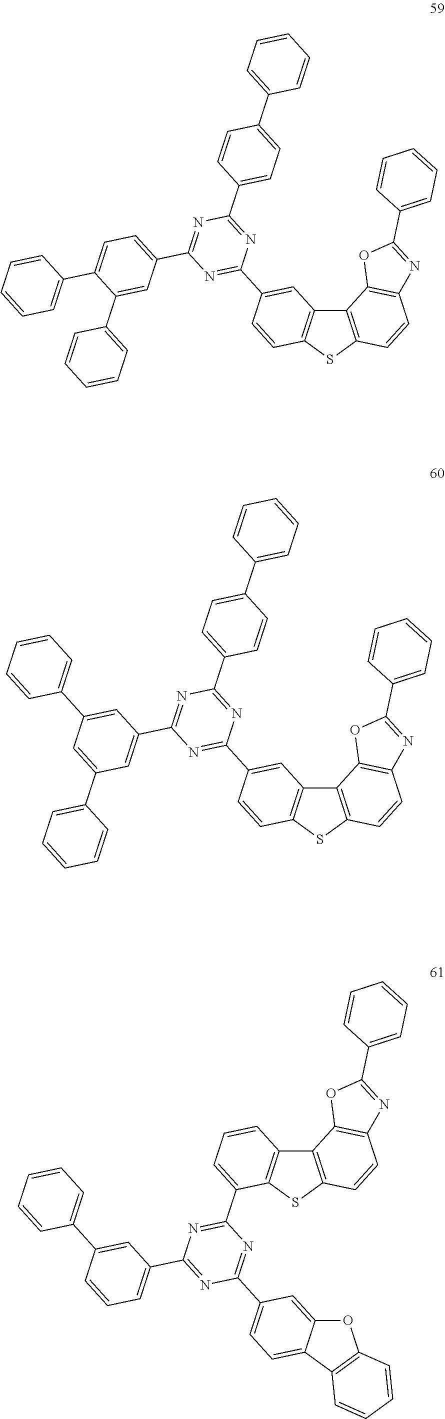 [SMILES: c1ccc(-c2ccc(-c3nc(-c4ccc(-c5ccccc5)c(-c5ccccc5)c4)nc(-c4ccc5sc6ccc7nc(-c8ccccc8)oc7c6c5c4)n3)cc2)cc1 ; c1ccc(-c2ccc(-c3nc(-c4cc(-c5ccccc5)cc(-c5ccccc5)c4)nc(-c4ccc5sc6ccc7nc(-c8ccccc8)oc7c6c5c4)n3)cc2)cc1 ; c1ccc(-c2cccc(-c3nc(-c4ccc5c(c4)oc4ccccc45)nc(-c4cccc5c4sc4ccc6nc(-c7ccccc7)oc6c45)n3)c2)cc1]

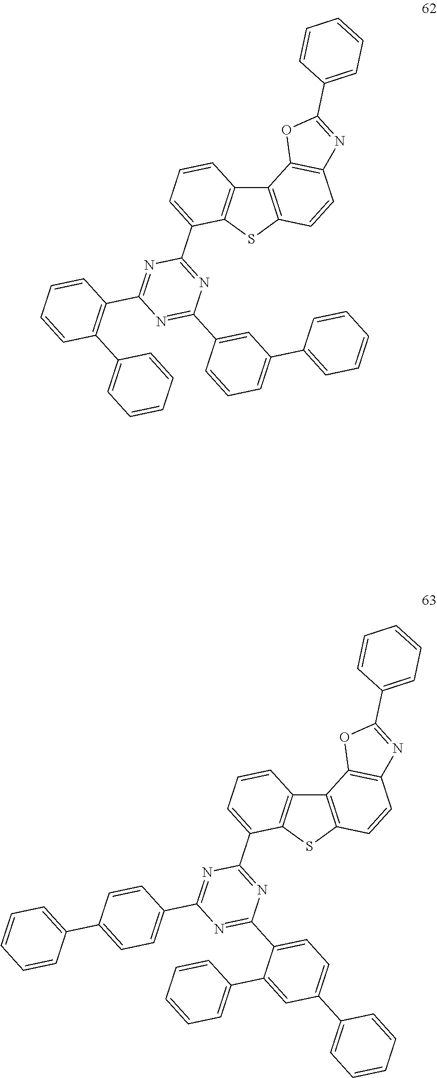 [SMILES: c1ccc(-c2cccc(-c3nc(-c4ccccc4-c4ccccc4)nc(-c4cccc5c4sc4ccc6nc(-c7ccccc7)oc6c45)n3)c2)cc1 ; c1ccc(-c2ccc(-c3nc(-c4ccc(-c5ccccc5)cc4-c4ccccc4)nc(-c4cccc5c4sc4ccc6nc(-c7ccccc7)oc6c45)n3)cc2)cc1]

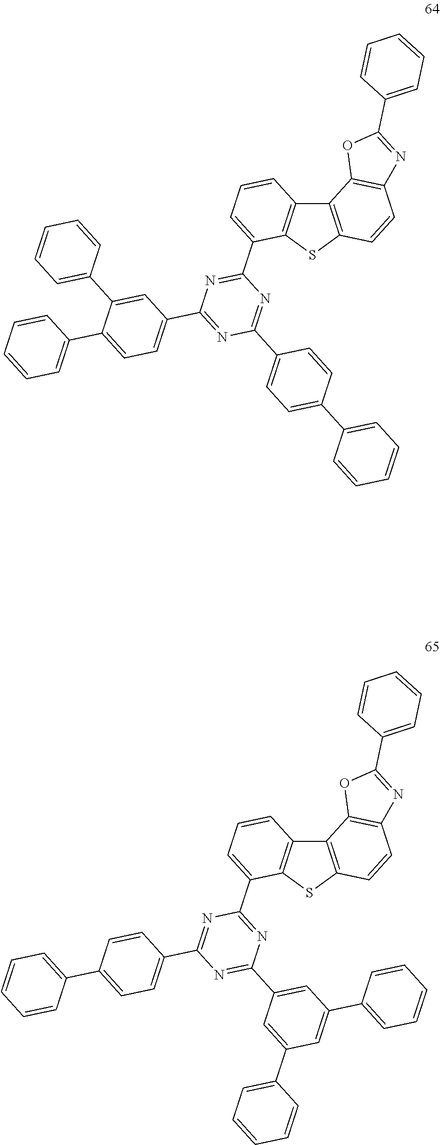 [SMILES: c1ccc(-c2ccc(-c3nc(-c4ccc(-c5ccccc5)c(-c5ccccc5)c4)nc(-c4cccc5c4sc4ccc6nc(-c7ccccc7)oc6c45)n3)cc2)cc1 ; c1ccc(-c2ccc(-c3nc(-c4cc(-c5ccccc5)cc(-c5ccccc5)c4)nc(-c4cccc5c4sc4ccc6nc(-c7ccccc7)oc6c45)n3)cc2)cc1]

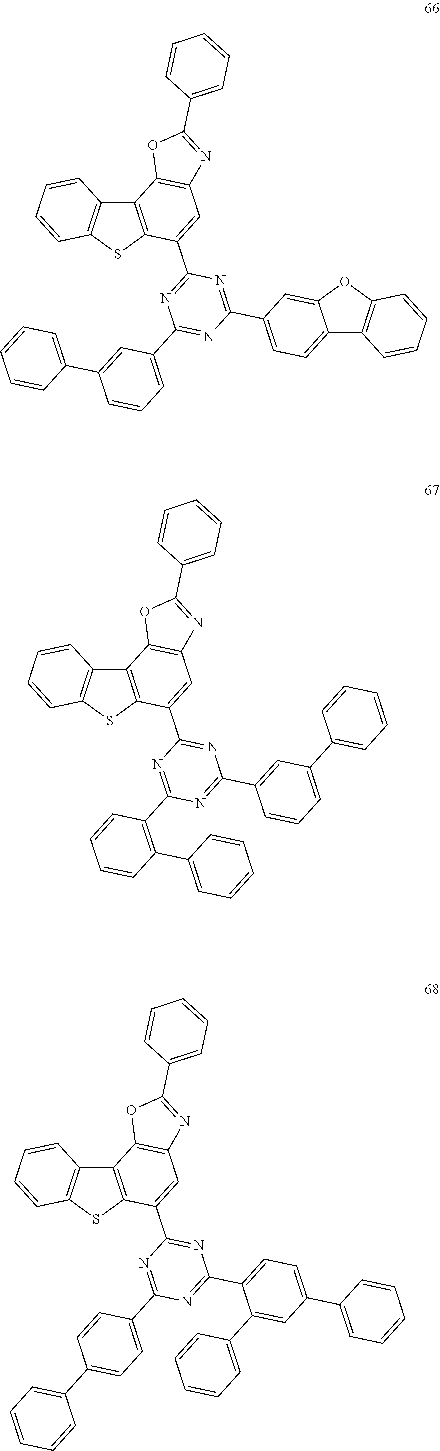 [SMILES: c1ccc(-c2cccc(-c3nc(-c4ccc5c(c4)oc4ccccc45)nc(-c4cc5nc(-c6ccccc6)oc5c5c4sc4ccccc45)n3)c2)cc1 ; c1ccc(-c2cccc(-c3nc(-c4ccccc4-c4ccccc4)nc(-c4cc5nc(-c6ccccc6)oc5c5c4sc4ccccc45)n3)c2)cc1 ; c1ccc(-c2ccc(-c3nc(-c4ccc(-c5ccccc5)cc4-c4ccccc4)nc(-c4cc5nc(-c6ccccc6)oc5c5c4sc4ccccc45)n3)cc2)cc1]

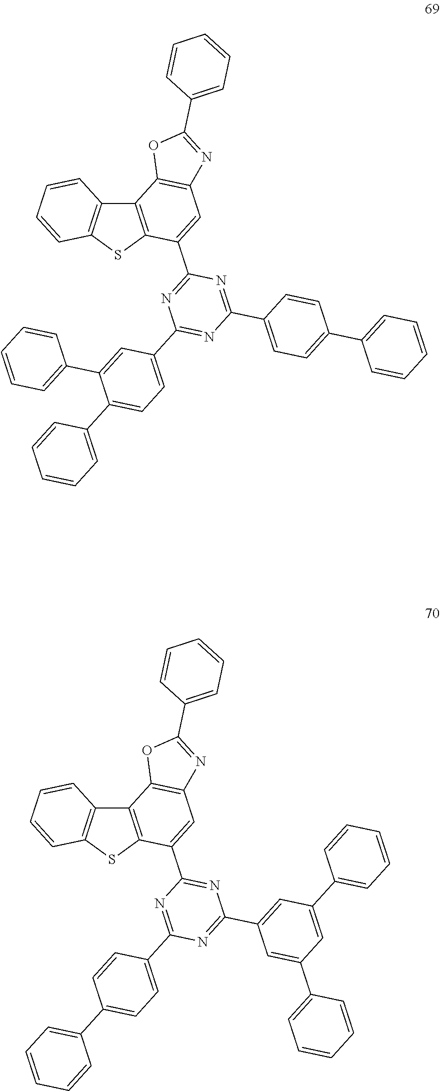 [SMILES: c1ccc(-c2ccc(-c3nc(-c4ccc(-c5ccccc5)c(-c5ccccc5)c4)nc(-c4cc5nc(-c6ccccc6)oc5c5c4sc4ccccc45)n3)cc2)cc1 ; c1ccc(-c2ccc(-c3nc(-c4cc(-c5ccccc5)cc(-c5ccccc5)c4)nc(-c4cc5nc(-c6ccccc6)oc5c5c4sc4ccccc45)n3)cc2)cc1]

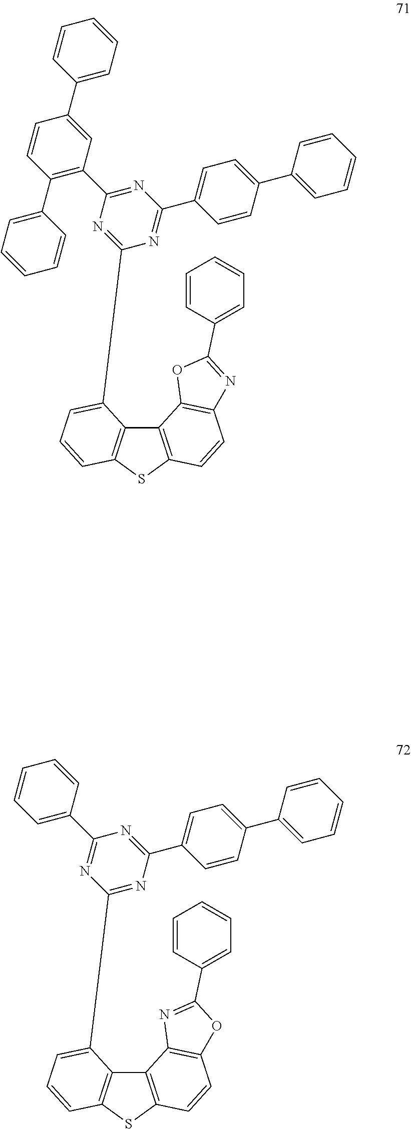 [SMILES: c1ccc(-c2ccc(-c3nc(-c4cc(-c5ccccc5)ccc4-c4ccccc4)nc(-c4cccc5sc6ccc7nc(-c8ccccc8)oc7c6c45)n3)cc2)cc1 ; c1ccc(-c2ccc(-c3nc(-c4ccccc4)nc(-c4cccc5sc6ccc7oc(-c8ccccc8)nc7c6c45)n3)cc2)cc1]

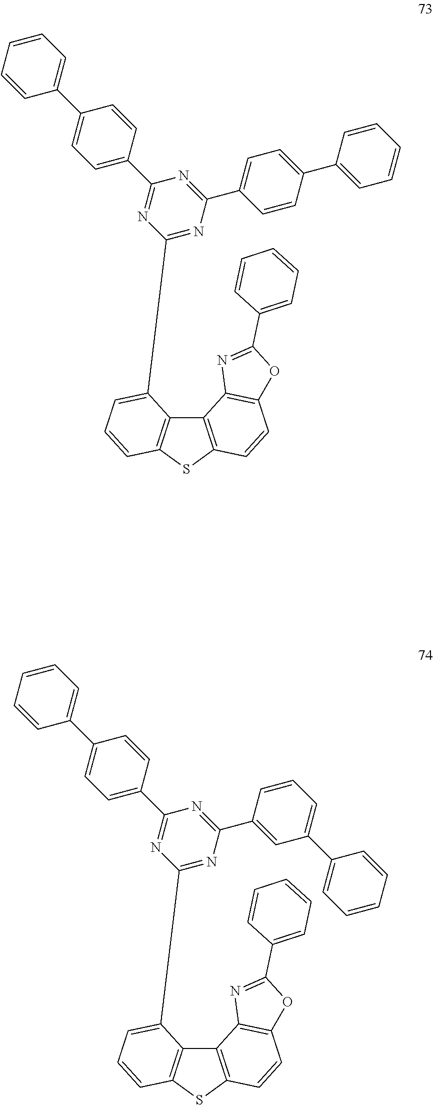 [SMILES: c1ccc(-c2ccc(-c3nc(-c4ccc(-c5ccccc5)cc4)nc(-c4cccc5sc6ccc7oc(-c8ccccc8)nc7c6c45)n3)cc2)cc1 ; c1ccc(-c2ccc(-c3nc(-c4cccc(-c5ccccc5)c4)nc(-c4cccc5sc6ccc7oc(-c8ccccc8)nc7c6c45)n3)cc2)cc1]

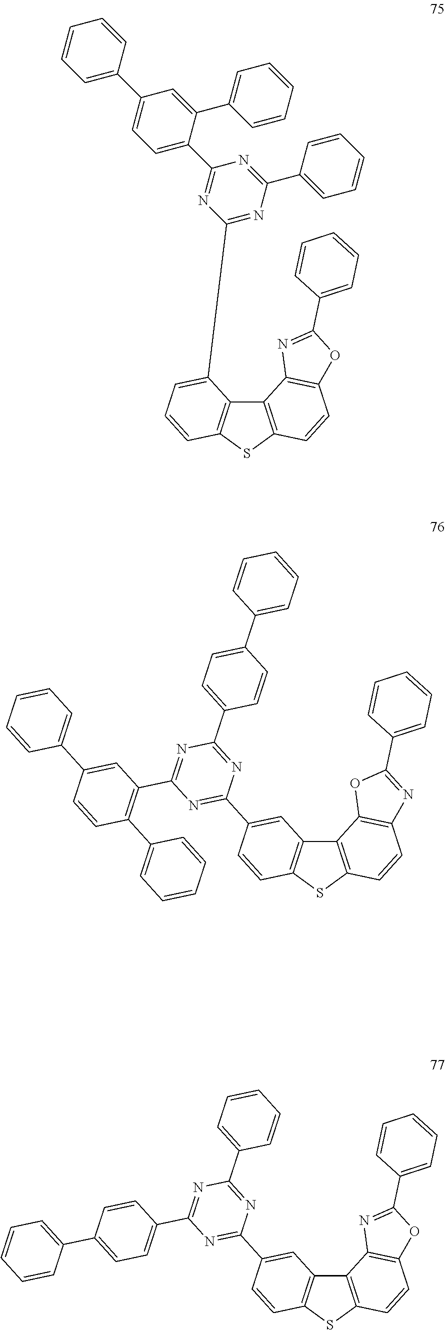 [SMILES: c1ccc(-c2ccc(-c3nc(-c4ccccc4)nc(-c4cccc5sc6ccc7oc(-c8ccccc8)nc7c6c45)n3)c(-c3ccccc3)c2)cc1 ; c1ccc(-c2ccc(-c3nc(-c4ccc5sc6ccc7nc(-c8ccccc8)oc7c6c5c4)nc(-c4cc(-c5ccccc5)ccc4-c4ccccc4)n3)cc2)cc1 ; c1ccc(-c2ccc(-c3nc(-c4ccccc4)nc(-c4ccc5sc6ccc7oc(-c8ccccc8)nc7c6c5c4)n3)cc2)cc1]

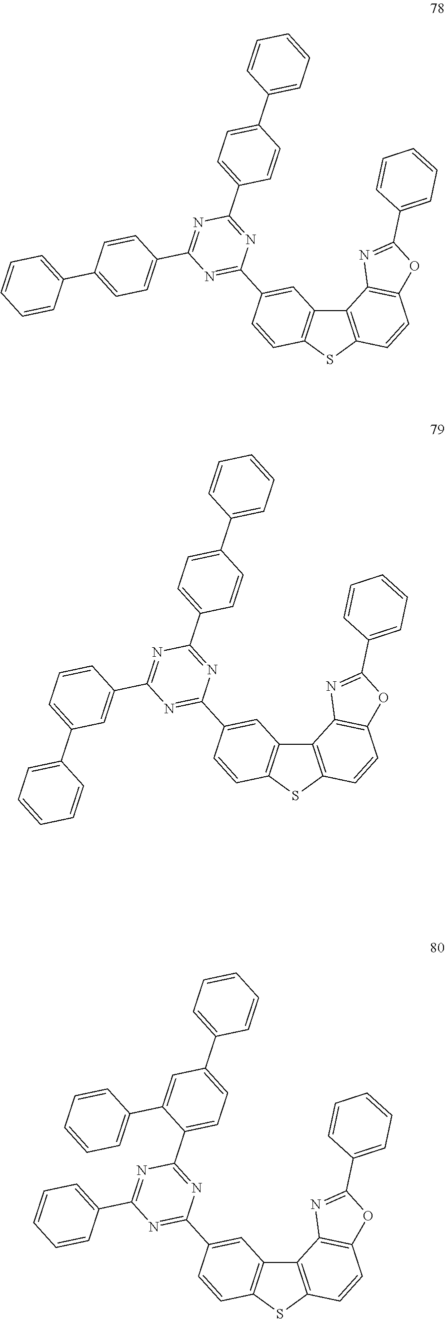 [SMILES: c1ccc(-c2ccc(-c3nc(-c4ccc(-c5ccccc5)cc4)nc(-c4ccc5sc6ccc7oc(-c8ccccc8)nc7c6c5c4)n3)cc2)cc1 ; c1ccc(-c2ccc(-c3nc(-c4cccc(-c5ccccc5)c4)nc(-c4ccc5sc6ccc7oc(-c8ccccc8)nc7c6c5c4)n3)cc2)cc1 ; c1ccc(-c2ccc(-c3nc(-c4ccccc4)nc(-c4ccc5sc6ccc7oc(-c8ccccc8)nc7c6c5c4)n3)c(-c3ccccc3)c2)cc1]

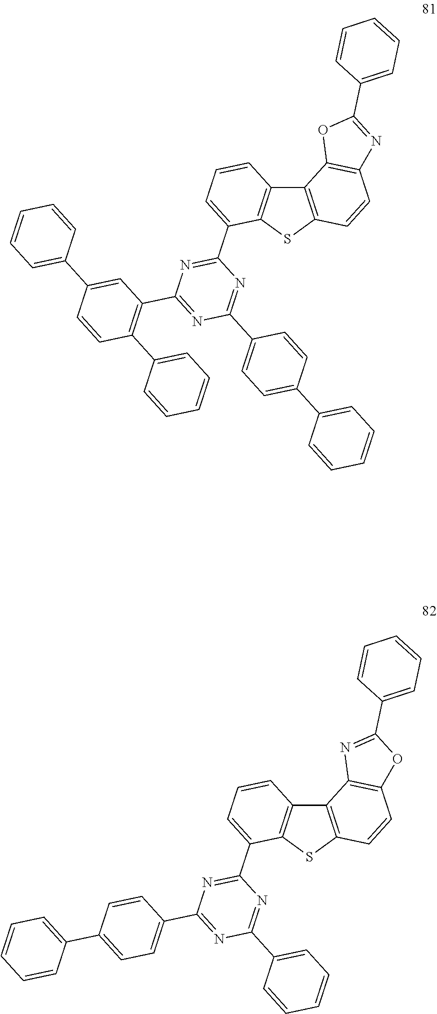 [SMILES: c1ccc(-c2ccc(-c3nc(-c4cc(-c5ccccc5)ccc4-c4ccccc4)nc(-c4cccc5c4sc4ccc6nc(-c7ccccc7)oc6c45)n3)cc2)cc1 ; c1ccc(-c2ccc(-c3nc(-c4ccccc4)nc(-c4cccc5c4sc4ccc6oc(-c7ccccc7)nc6c45)n3)cc2)cc1]

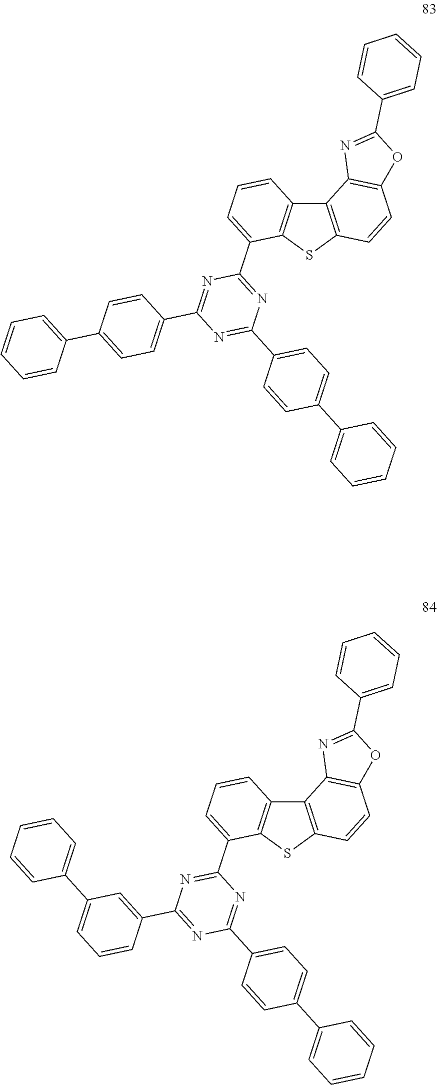 [SMILES: c1ccc(-c2ccc(-c3nc(-c4ccc(-c5ccccc5)cc4)nc(-c4cccc5c4sc4ccc6oc(-c7ccccc7)nc6c45)n3)cc2)cc1 ; c1ccc(-c2ccc(-c3nc(-c4cccc(-c5ccccc5)c4)nc(-c4cccc5c4sc4ccc6oc(-c7ccccc7)nc6c45)n3)cc2)cc1]

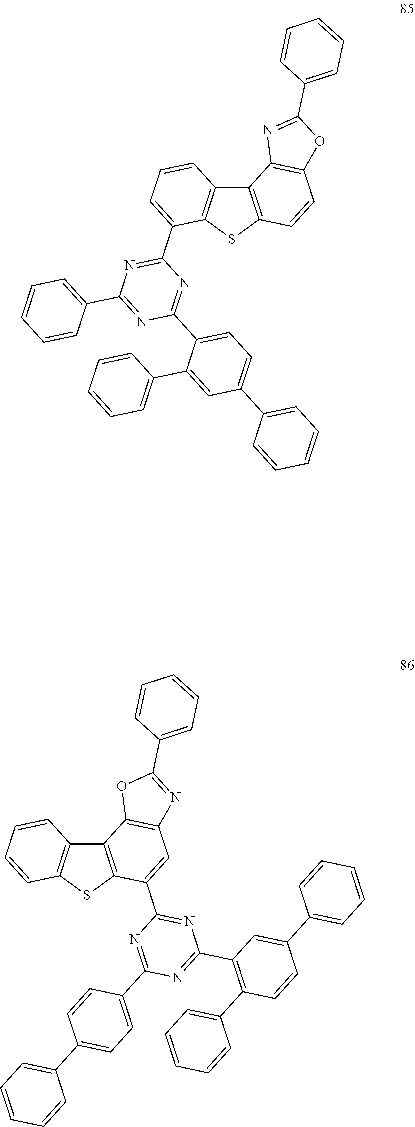 [SMILES: c1ccc(-c2ccc(-c3nc(-c4ccccc4)nc(-c4cccc5c4sc4ccc6oc(-c7ccccc7)nc6c45)n3)c(-c3ccccc3)c2)cc1 ; c1ccc(-c2ccc(-c3nc(-c4cc(-c5ccccc5)ccc4-c4ccccc4)nc(-c4cc5nc(-c6ccccc6)oc5c5c4sc4ccccc45)n3)cc2)cc1]

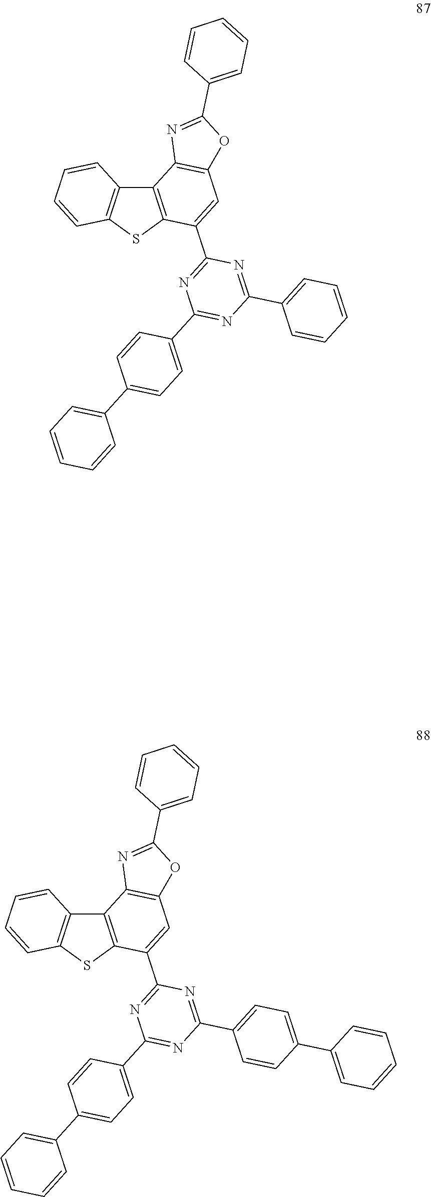 [SMILES: c1ccc(-c2ccc(-c3nc(-c4ccccc4)nc(-c4cc5oc(-c6ccccc6)nc5c5c4sc4ccccc45)n3)cc2)cc1 ; c1ccc(-c2ccc(-c3nc(-c4ccc(-c5ccccc5)cc4)nc(-c4cc5oc(-c6ccccc6)nc5c5c4sc4ccccc45)n3)cc2)cc1]

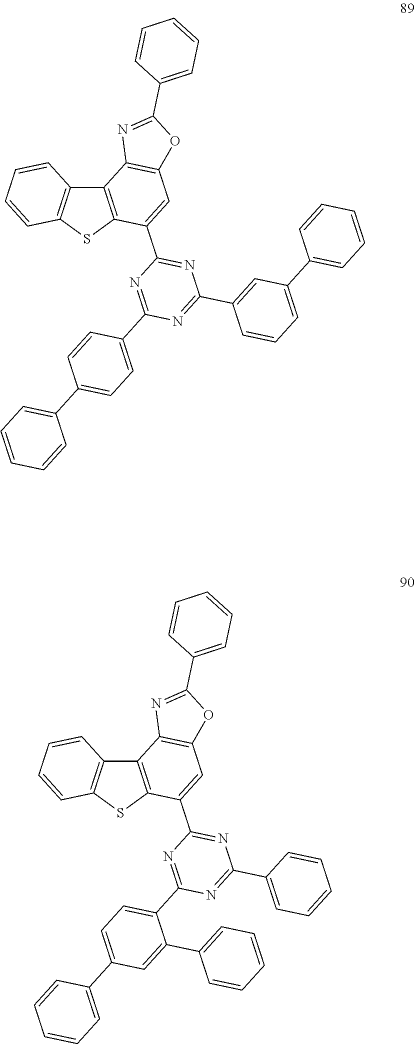 [SMILES: c1ccc(-c2ccc(-c3nc(-c4cccc(-c5ccccc5)c4)nc(-c4cc5oc(-c6ccccc6)nc5c5c4sc4ccccc45)n3)cc2)cc1 ; c1ccc(-c2ccc(-c3nc(-c4ccccc4)nc(-c4cc5oc(-c6ccccc6)nc5c5c4sc4ccccc45)n3)c(-c3ccccc3)c2)cc1]

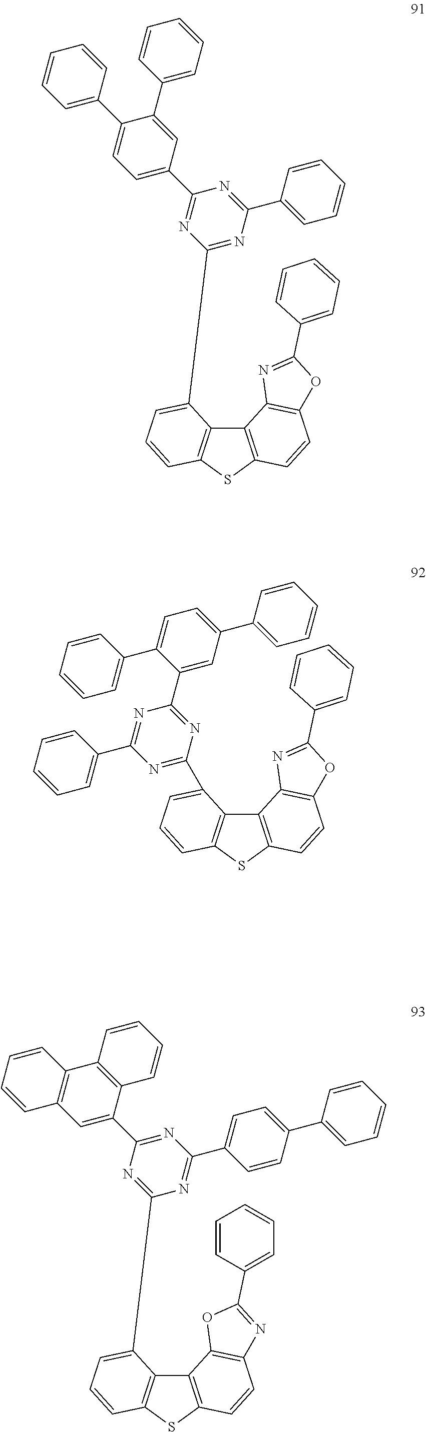 [SMILES: c1ccc(-c2nc(-c3ccc(-c4ccccc4)c(-c4ccccc4)c3)nc(-c3cccc4sc5ccc6oc(-c7ccccc7)nc6c5c34)n2)cc1 ; c1ccc(-c2ccc(-c3ccccc3)c(-c3nc(-c4ccccc4)nc(-c4cccc5sc6ccc7oc(-c8ccccc8)nc7c6c45)n3)c2)cc1 ; c1ccc(-c2ccc(-c3nc(-c4cc5ccccc5c5ccccc45)nc(-c4cccc5sc6ccc7nc(-c8ccccc8)oc7c6c45)n3)cc2)cc1]

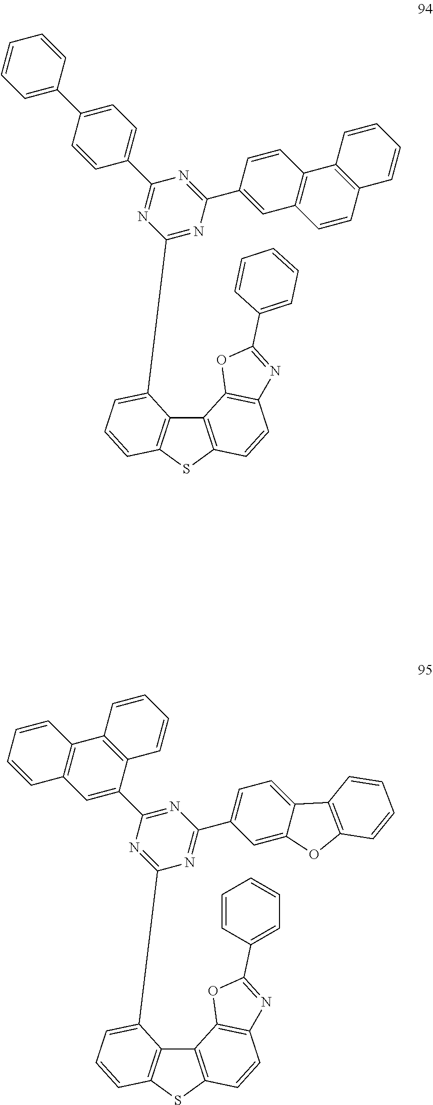 [SMILES: c1ccc(-c2ccc(-c3nc(-c4ccc5c(ccc6ccccc65)c4)nc(-c4cccc5sc6ccc7nc(-c8ccccc8)oc7c6c45)n3)cc2)cc1 ; c1ccc(-c2nc3ccc4sc5cccc(-c6nc(-c7ccc8c(c7)oc7ccccc78)nc(-c7cc8ccccc8c8ccccc78)n6)c5c4c3o2)cc1]

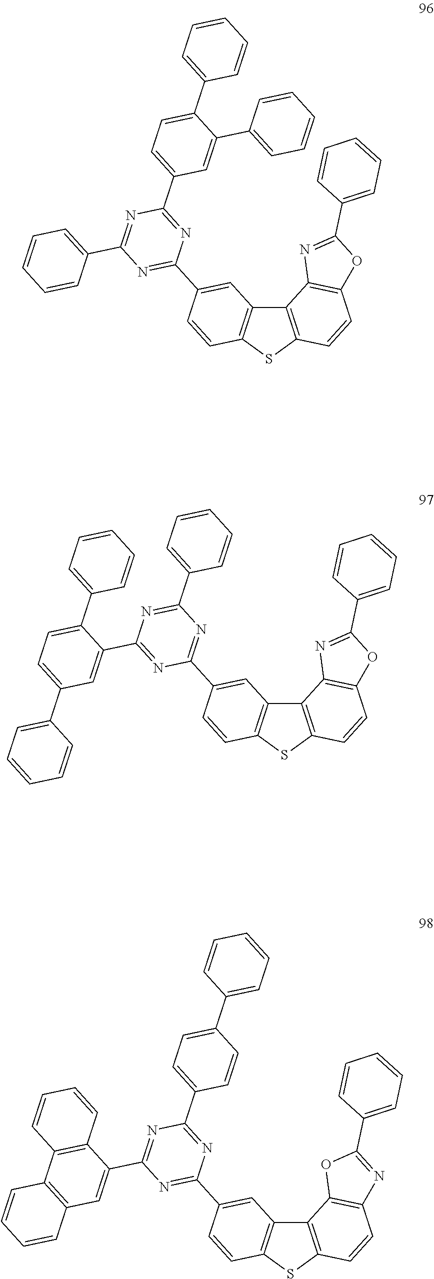 [SMILES: c1ccc(-c2nc(-c3ccc(-c4ccccc4)c(-c4ccccc4)c3)nc(-c3ccc4sc5ccc6oc(-c7ccccc7)nc6c5c4c3)n2)cc1 ; c1ccc(-c2ccc(-c3ccccc3)c(-c3nc(-c4ccccc4)nc(-c4ccc5sc6ccc7oc(-c8ccccc8)nc7c6c5c4)n3)c2)cc1 ; c1ccc(-c2ccc(-c3nc(-c4ccc5sc6ccc7nc(-c8ccccc8)oc7c6c5c4)nc(-c4cc5ccccc5c5ccccc45)n3)cc2)cc1]

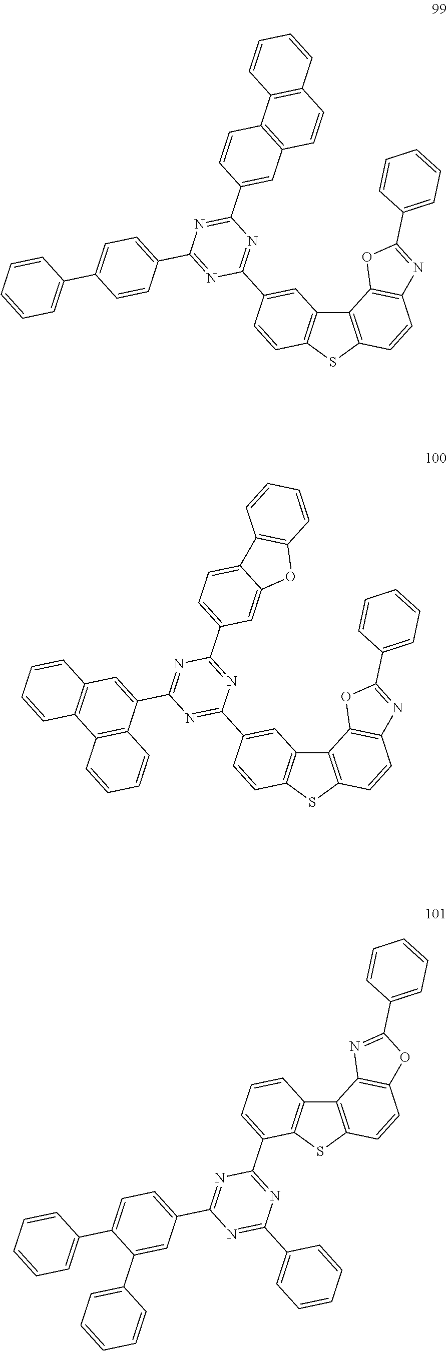 [SMILES: c1ccc(-c2ccc(-c3nc(-c4ccc5c(ccc6ccccc65)c4)nc(-c4ccc5sc6ccc7nc(-c8ccccc8)oc7c6c5c4)n3)cc2)cc1 ; c1ccc(-c2nc3ccc4sc5ccc(-c6nc(-c7ccc8c(c7)oc7ccccc78)nc(-c7cc8ccccc8c8ccccc78)n6)cc5c4c3o2)cc1 ; c1ccc(-c2nc(-c3ccc(-c4ccccc4)c(-c4ccccc4)c3)nc(-c3cccc4c3sc3ccc5oc(-c6ccccc6)nc5c34)n2)cc1]

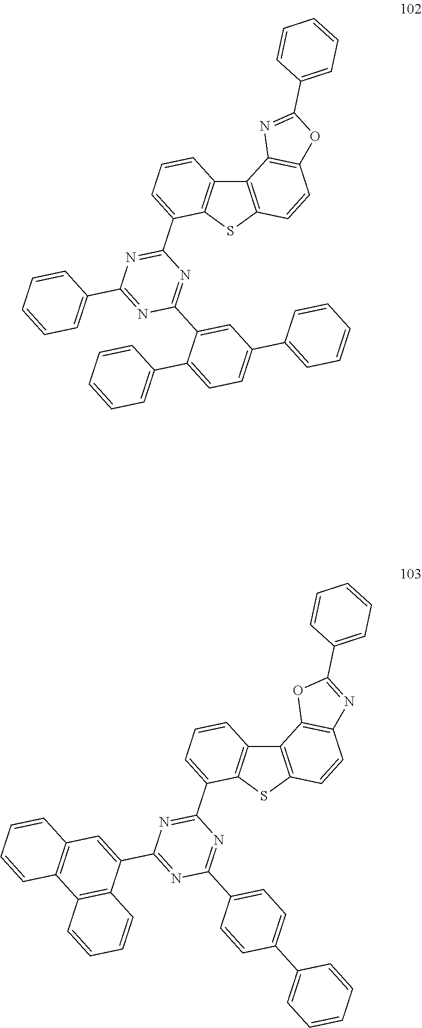 [SMILES: c1ccc(-c2ccc(-c3ccccc3)c(-c3nc(-c4ccccc4)nc(-c4cccc5c4sc4ccc6oc(-c7ccccc7)nc6c45)n3)c2)cc1 ; c1ccc(-c2ccc(-c3nc(-c4cc5ccccc5c5ccccc45)nc(-c4cccc5c4sc4ccc6nc(-c7ccccc7)oc6c45)n3)cc2)cc1]

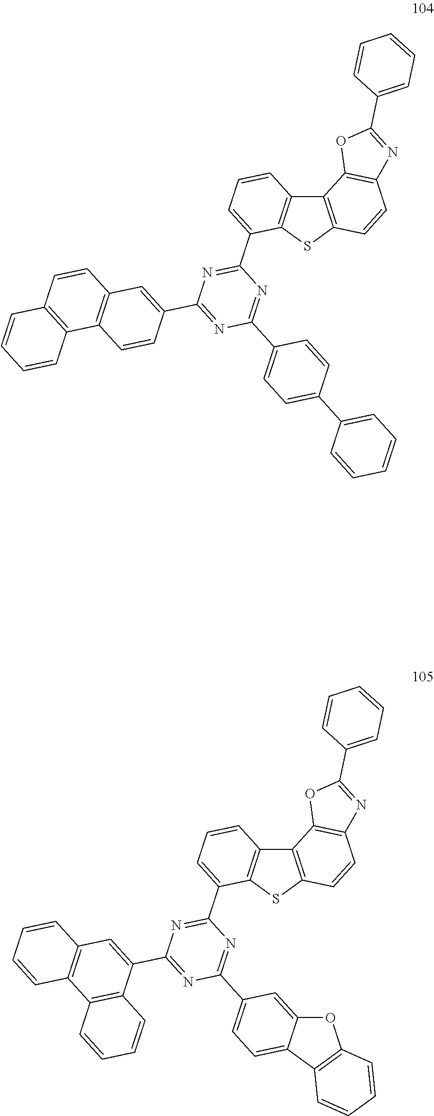 [SMILES: c1ccc(-c2ccc(-c3nc(-c4ccc5c(ccc6ccccc65)c4)nc(-c4cccc5c4sc4ccc6nc(-c7ccccc7)oc6c45)n3)cc2)cc1 ; c1ccc(-c2nc3ccc4sc5c(-c6nc(-c7ccc8c(c7)oc7ccccc78)nc(-c7cc8ccccc8c8ccccc78)n6)cccc5c4c3o2)cc1]

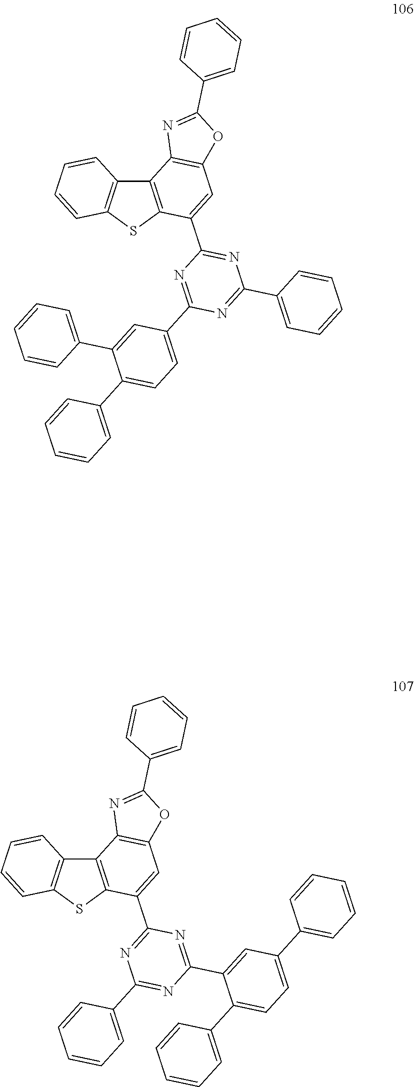 [SMILES: c1ccc(-c2nc(-c3ccc(-c4ccccc4)c(-c4ccccc4)c3)nc(-c3cc4oc(-c5ccccc5)nc4c4c3sc3ccccc34)n2)cc1 ; c1ccc(-c2ccc(-c3ccccc3)c(-c3nc(-c4ccccc4)nc(-c4cc5oc(-c6ccccc6)nc5c5c4sc4ccccc45)n3)c2)cc1]

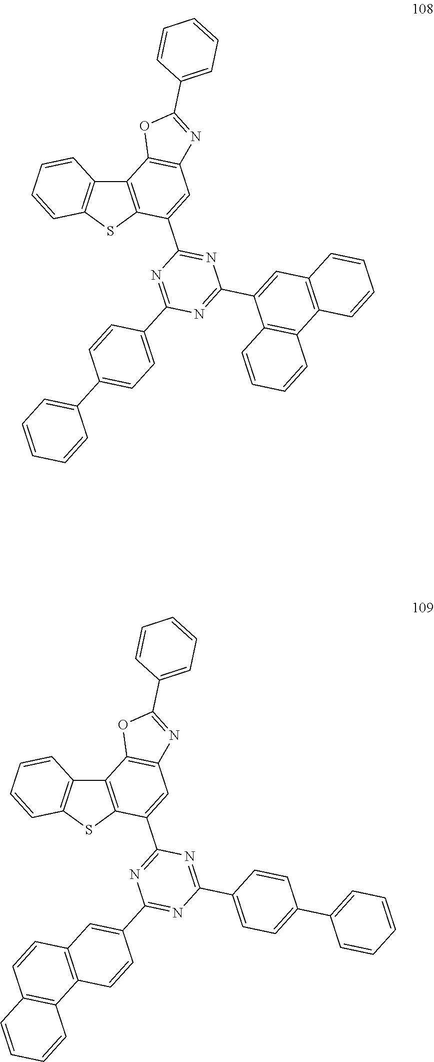 [SMILES: c1ccc(-c2ccc(-c3nc(-c4cc5ccccc5c5ccccc45)nc(-c4cc5nc(-c6ccccc6)oc5c5c4sc4ccccc45)n3)cc2)cc1 ; c1ccc(-c2ccc(-c3nc(-c4ccc5c(ccc6ccccc65)c4)nc(-c4cc5nc(-c6ccccc6)oc5c5c4sc4ccccc45)n3)cc2)cc1]

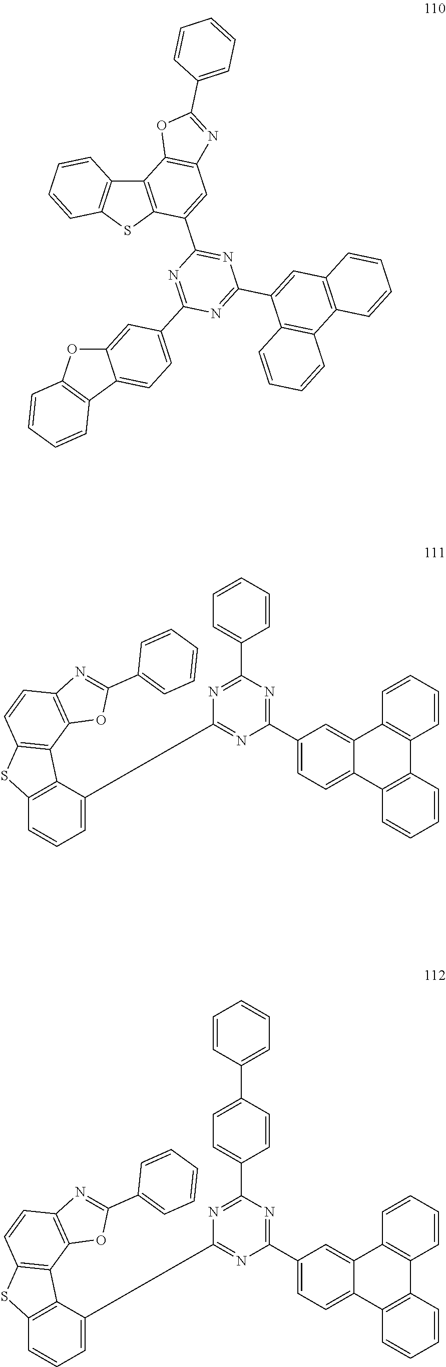 [SMILES: c1ccc(-c2nc3cc(-c4nc(-c5ccc6c(c5)oc5ccccc56)nc(-c5cc6ccccc6c6ccccc56)n4)c4sc5ccccc5c4c3o2)cc1 ; c1ccc(-c2nc(-c3ccc4c5ccccc5c5ccccc5c4c3)nc(-c3cccc4sc5ccc6nc(-c7ccccc7)oc6c5c34)n2)cc1 ; c1ccc(-c2ccc(-c3nc(-c4ccc5c6ccccc6c6ccccc6c5c4)nc(-c4cccc5sc6ccc7nc(-c8ccccc8)oc7c6c45)n3)cc2)cc1]

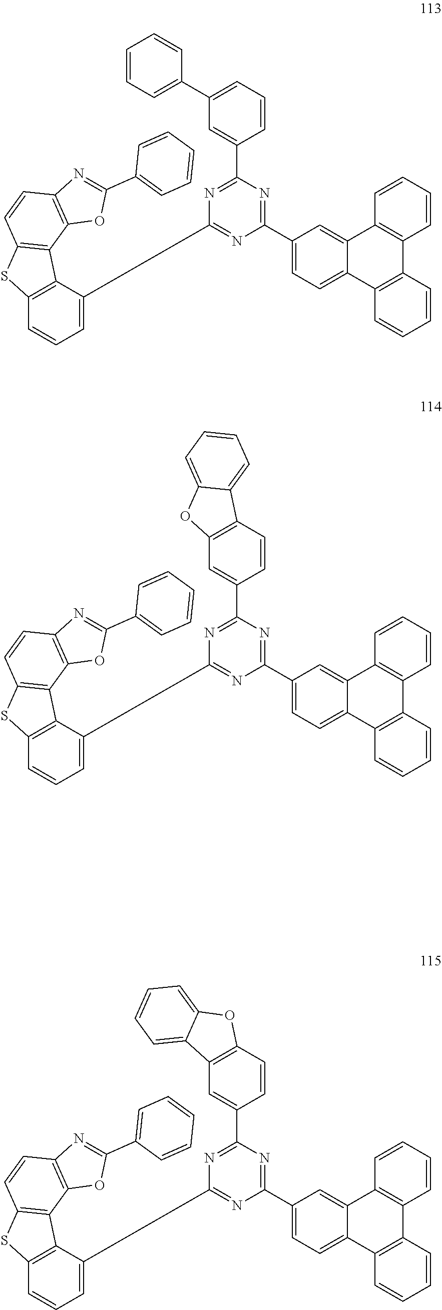 [SMILES: c1ccc(-c2cccc(-c3nc(-c4ccc5c6ccccc6c6ccccc6c5c4)nc(-c4cccc5sc6ccc7nc(-c8ccccc8)oc7c6c45)n3)c2)cc1 ; c1ccc(-c2nc3ccc4sc5cccc(-c6nc(-c7ccc8c(c7)oc7ccccc78)nc(-c7ccc8c9ccccc9c9ccccc9c8c7)n6)c5c4c3o2)cc1 ; c1ccc(-c2nc3ccc4sc5cccc(-c6nc(-c7ccc8oc9ccccc9c8c7)nc(-c7ccc8c9ccccc9c9ccccc9c8c7)n6)c5c4c3o2)cc1]

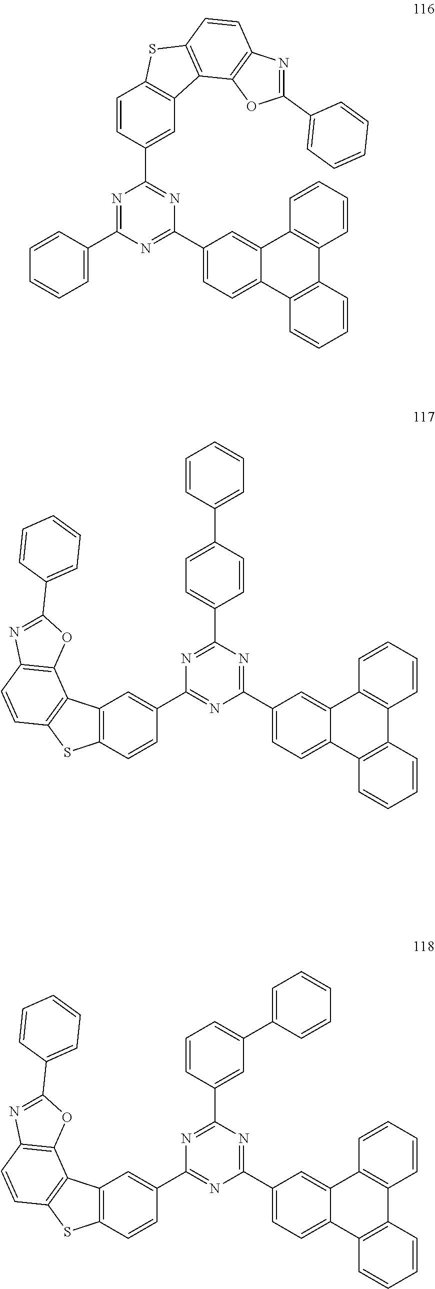 [SMILES: c1ccc(-c2nc(-c3ccc4c5ccccc5c5ccccc5c4c3)nc(-c3ccc4sc5ccc6nc(-c7ccccc7)oc6c5c4c3)n2)cc1 ; c1ccc(-c2ccc(-c3nc(-c4ccc5c6ccccc6c6ccccc6c5c4)nc(-c4ccc5sc6ccc7nc(-c8ccccc8)oc7c6c5c4)n3)cc2)cc1 ; c1ccc(-c2cccc(-c3nc(-c4ccc5c6ccccc6c6ccccc6c5c4)nc(-c4ccc5sc6ccc7nc(-c8ccccc8)oc7c6c5c4)n3)c2)cc1]

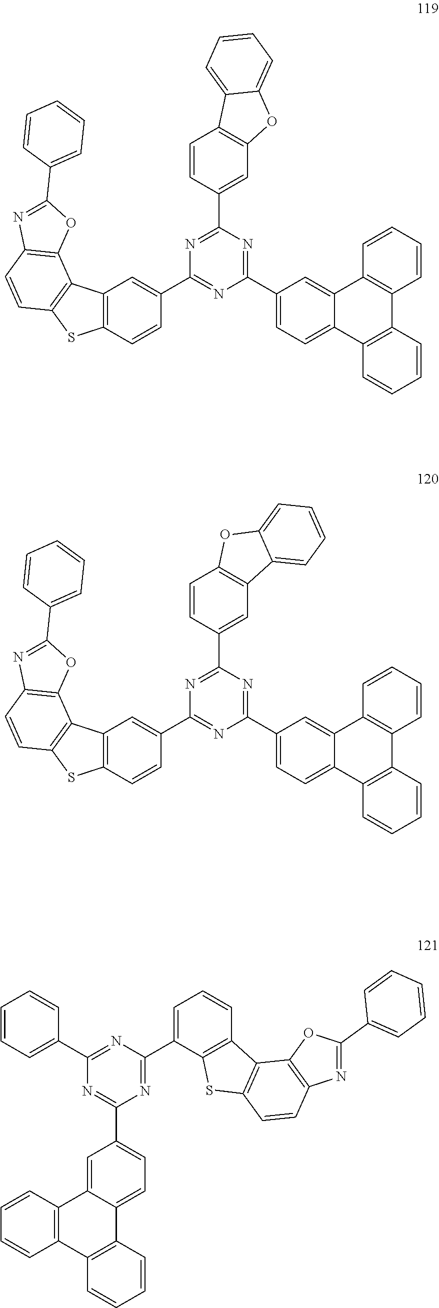 [SMILES: c1ccc(-c2nc3ccc4sc5ccc(-c6nc(-c7ccc8c(c7)oc7ccccc78)nc(-c7ccc8c9ccccc9c9ccccc9c8c7)n6)cc5c4c3o2)cc1 ; c1ccc(-c2nc3ccc4sc5ccc(-c6nc(-c7ccc8oc9ccccc9c8c7)nc(-c7ccc8c9ccccc9c9ccccc9c8c7)n6)cc5c4c3o2)cc1 ; c1ccc(-c2nc(-c3ccc4c5ccccc5c5ccccc5c4c3)nc(-c3cccc4c3sc3ccc5nc(-c6ccccc6)oc5c34)n2)cc1]

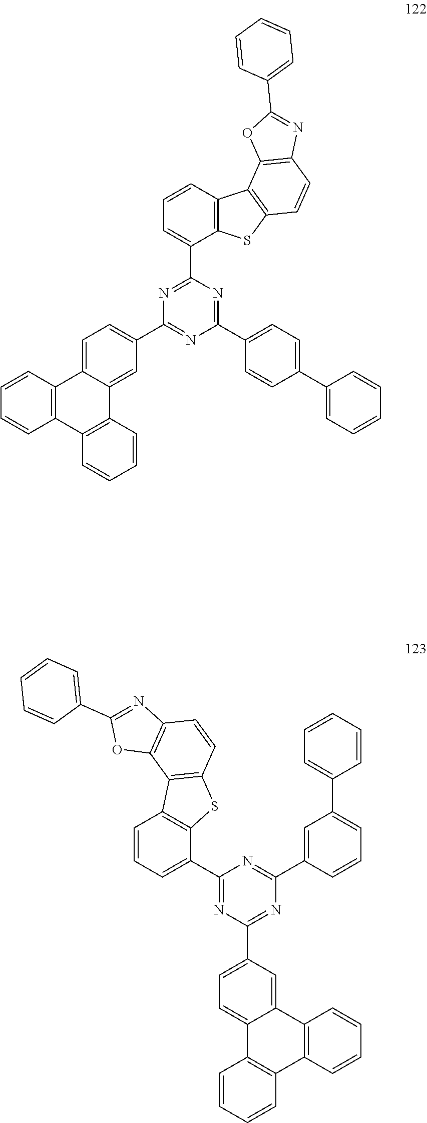 [SMILES: c1ccc(-c2ccc(-c3nc(-c4ccc5c6ccccc6c6ccccc6c5c4)nc(-c4cccc5c4sc4ccc6nc(-c7ccccc7)oc6c45)n3)cc2)cc1 ; c1ccc(-c2cccc(-c3nc(-c4ccc5c6ccccc6c6ccccc6c5c4)nc(-c4cccc5c4sc4ccc6nc(-c7ccccc7)oc6c45)n3)c2)cc1]

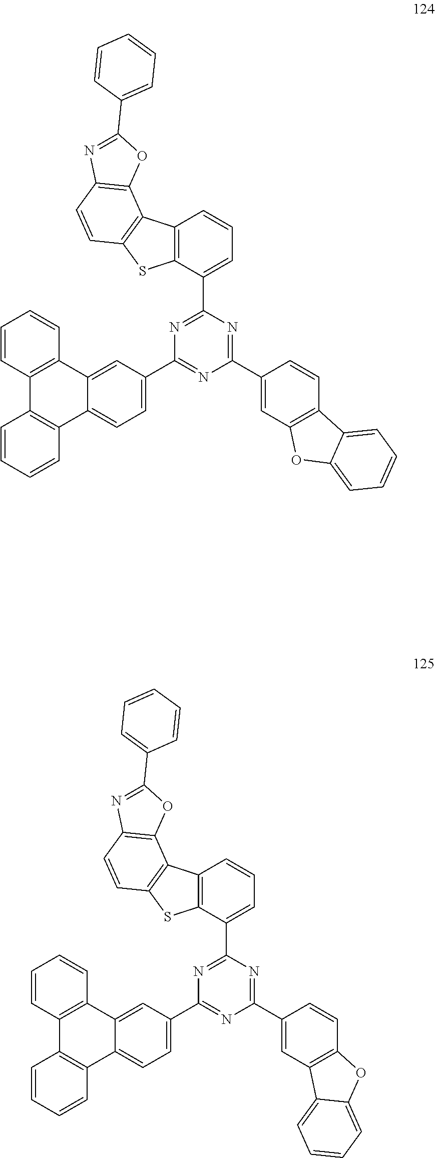 [SMILES: c1ccc(-c2nc3ccc4sc5c(-c6nc(-c7ccc8c(c7)oc7ccccc78)nc(-c7ccc8c9ccccc9c9ccccc9c8c7)n6)cccc5c4c3o2)cc1 ; c1ccc(-c2nc3ccc4sc5c(-c6nc(-c7ccc8oc9ccccc9c8c7)nc(-c7ccc8c9ccccc9c9ccccc9c8c7)n6)cccc5c4c3o2)cc1]

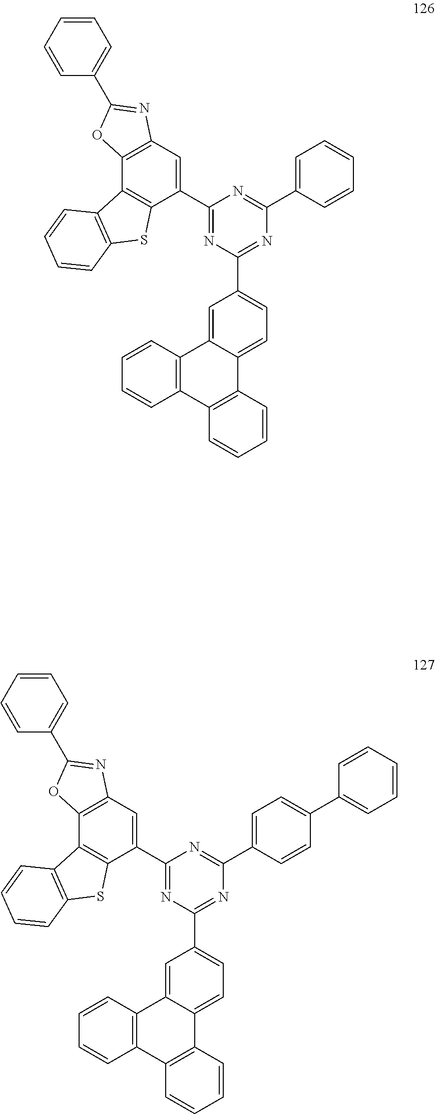 [SMILES: c1ccc(-c2nc(-c3ccc4c5ccccc5c5ccccc5c4c3)nc(-c3cc4nc(-c5ccccc5)oc4c4c3sc3ccccc34)n2)cc1 ; c1ccc(-c2ccc(-c3nc(-c4ccc5c6ccccc6c6ccccc6c5c4)nc(-c4cc5nc(-c6ccccc6)oc5c5c4sc4ccccc45)n3)cc2)cc1]

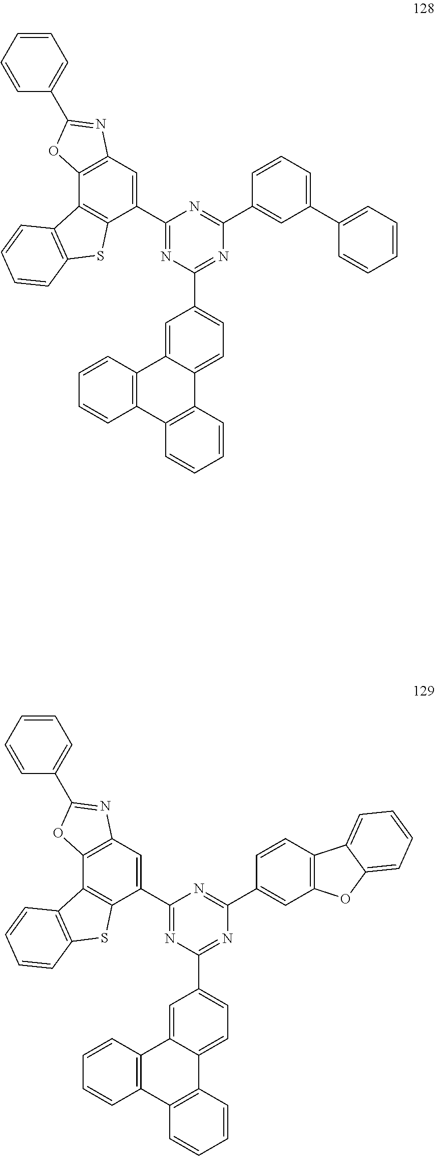 [SMILES: c1ccc(-c2cccc(-c3nc(-c4ccc5c6ccccc6c6ccccc6c5c4)nc(-c4cc5nc(-c6ccccc6)oc5c5c4sc4ccccc45)n3)c2)cc1 ; c1ccc(-c2nc3cc(-c4nc(-c5ccc6c(c5)oc5ccccc56)nc(-c5ccc6c7ccccc7c7ccccc7c6c5)n4)c4sc5ccccc5c4c3o2)cc1]

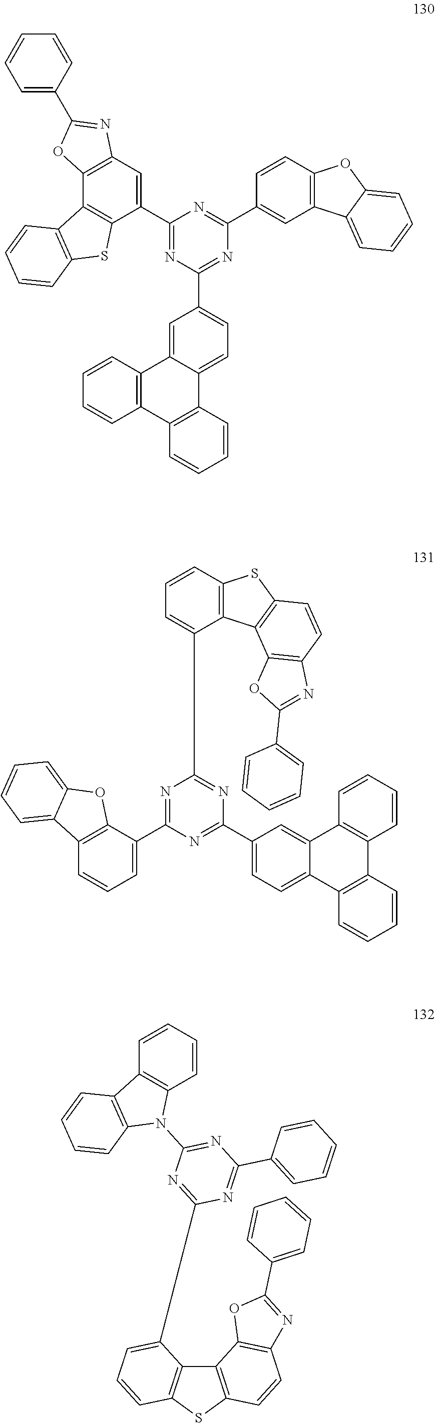 [SMILES: c1ccc(-c2nc3cc(-c4nc(-c5ccc6oc7ccccc7c6c5)nc(-c5ccc6c7ccccc7c7ccccc7c6c5)n4)c4sc5ccccc5c4c3o2)cc1 ; c1ccc(-c2nc3ccc4sc5cccc(-c6nc(-c7ccc8c9ccccc9c9ccccc9c8c7)nc(-c7cccc8c7oc7ccccc78)n6)c5c4c3o2)cc1 ; c1ccc(-c2nc(-c3cccc4sc5ccc6nc(-c7ccccc7)oc6c5c34)nc(-n3c4ccccc4c4ccccc43)n2)cc1]

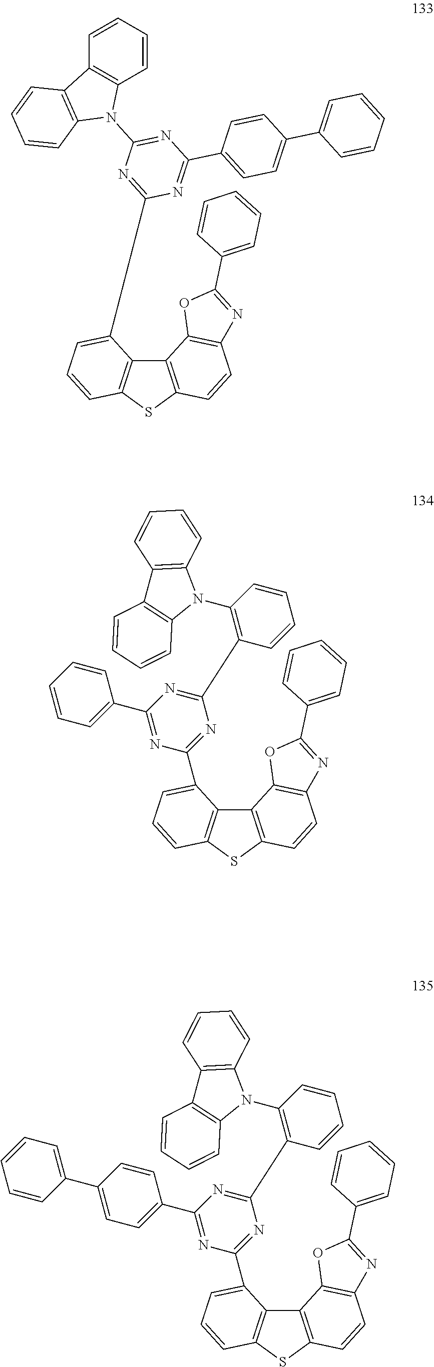 [SMILES: c1ccc(-c2ccc(-c3nc(-c4cccc5sc6ccc7nc(-c8ccccc8)oc7c6c45)nc(-n4c5ccccc5c5ccccc54)n3)cc2)cc1 ; c1ccc(-c2nc(-c3ccccc3-n3c4ccccc4c4ccccc43)nc(-c3cccc4sc5ccc6nc(-c7ccccc7)oc6c5c34)n2)cc1 ; c1ccc(-c2ccc(-c3nc(-c4ccccc4-n4c5ccccc5c5ccccc54)nc(-c4cccc5sc6ccc7nc(-c8ccccc8)oc7c6c45)n3)cc2)cc1]

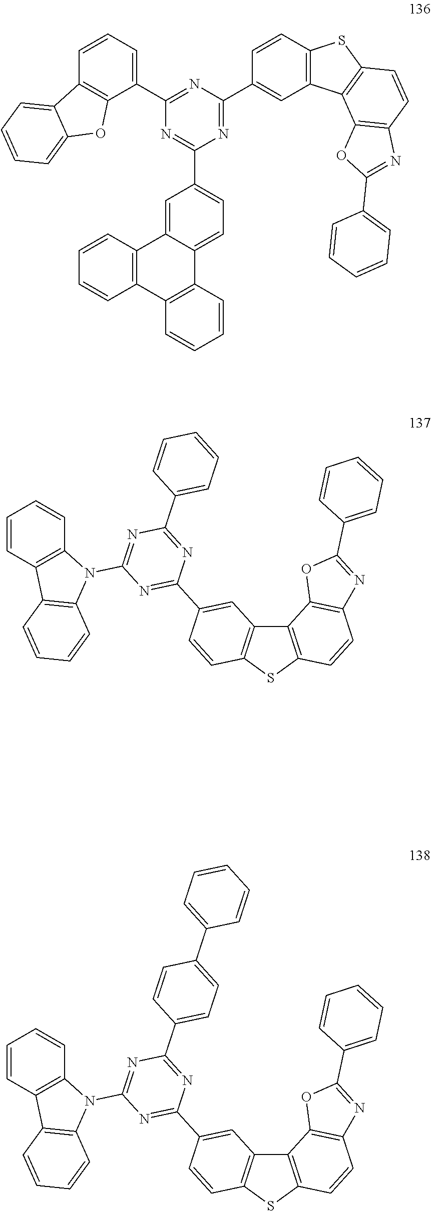 [SMILES: c1ccc(-c2nc3ccc4sc5ccc(-c6nc(-c7ccc8c9ccccc9c9ccccc9c8c7)nc(-c7cccc8c7oc7ccccc78)n6)cc5c4c3o2)cc1 ; c1ccc(-c2nc(-c3ccc4sc5ccc6nc(-c7ccccc7)oc6c5c4c3)nc(-n3c4ccccc4c4ccccc43)n2)cc1 ; c1ccc(-c2ccc(-c3nc(-c4ccc5sc6ccc7nc(-c8ccccc8)oc7c6c5c4)nc(-n4c5ccccc5c5ccccc54)n3)cc2)cc1]

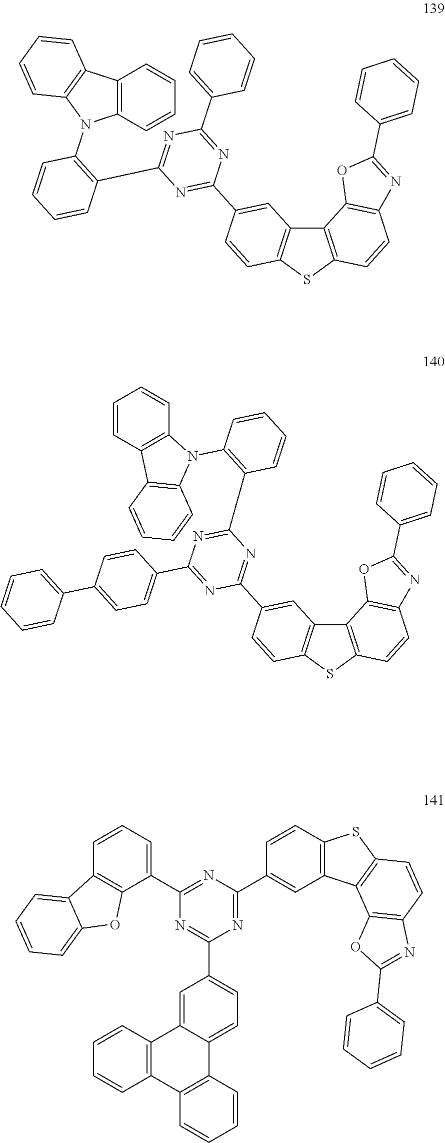 [SMILES: c1ccc(-c2nc(-c3ccc4sc5ccc6nc(-c7ccccc7)oc6c5c4c3)nc(-c3ccccc3-n3c4ccccc4c4ccccc43)n2)cc1 ; c1ccc(-c2ccc(-c3nc(-c4ccc5sc6ccc7nc(-c8ccccc8)oc7c6c5c4)nc(-c4ccccc4-n4c5ccccc5c5ccccc54)n3)cc2)cc1 ; c1ccc(-c2nc3ccc4sc5ccc(-c6nc(-c7ccc8c9ccccc9c9ccccc9c8c7)nc(-c7cccc8c7oc7ccccc78)n6)cc5c4c3o2)cc1]

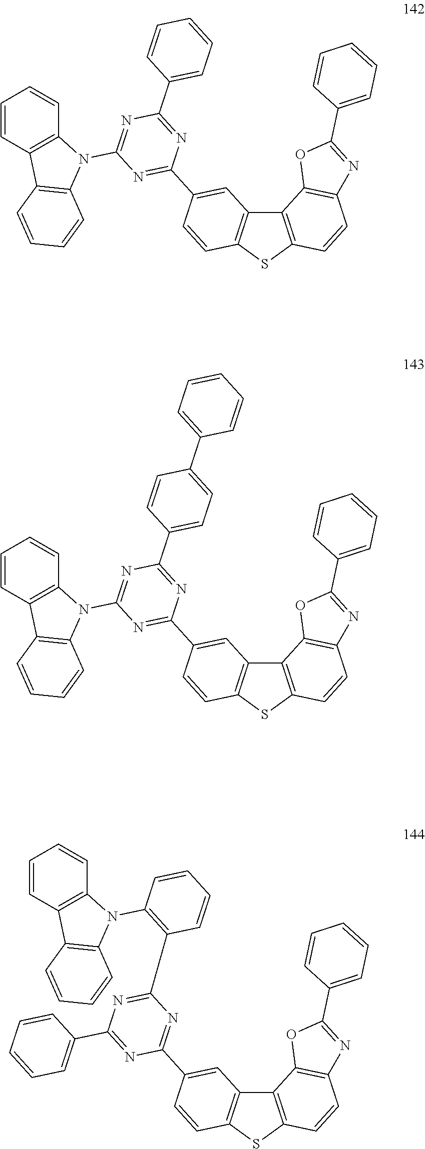 [SMILES: c1ccc(-c2nc(-c3ccc4sc5ccc6nc(-c7ccccc7)oc6c5c4c3)nc(-n3c4ccccc4c4ccccc43)n2)cc1 ; c1ccc(-c2ccc(-c3nc(-c4ccc5sc6ccc7nc(-c8ccccc8)oc7c6c5c4)nc(-n4c5ccccc5c5ccccc54)n3)cc2)cc1 ; c1ccc(-c2nc(-c3ccc4sc5ccc6nc(-c7ccccc7)oc6c5c4c3)nc(-c3ccccc3-n3c4ccccc4c4ccccc43)n2)cc1]

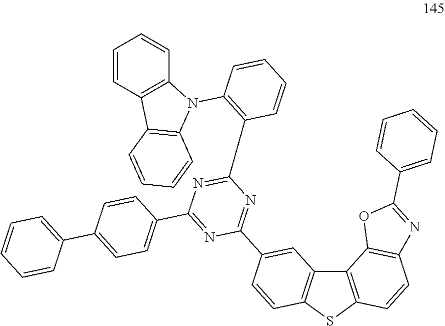 [SMILES: c1ccc(-c2ccc(-c3nc(-c4ccc5sc6ccc7nc(-c8ccccc8)oc7c6c5c4)nc(-c4ccccc4-n4c5ccccc5c5ccccc54)n3)cc2)cc1]

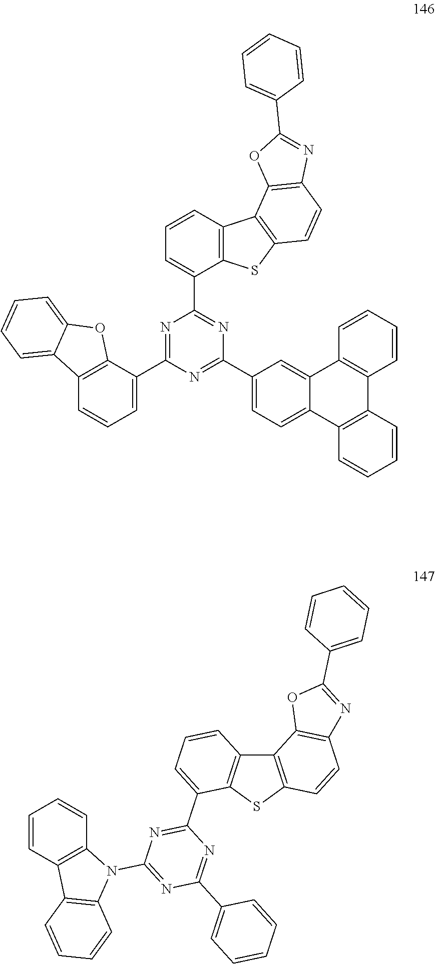 [SMILES: c1ccc(-c2nc3ccc4sc5c(-c6nc(-c7ccc8c9ccccc9c9ccccc9c8c7)nc(-c7cccc8c7oc7ccccc78)n6)cccc5c4c3o2)cc1 ; c1ccc(-c2nc(-c3cccc4c3sc3ccc5nc(-c6ccccc6)oc5c34)nc(-n3c4ccccc4c4ccccc43)n2)cc1]

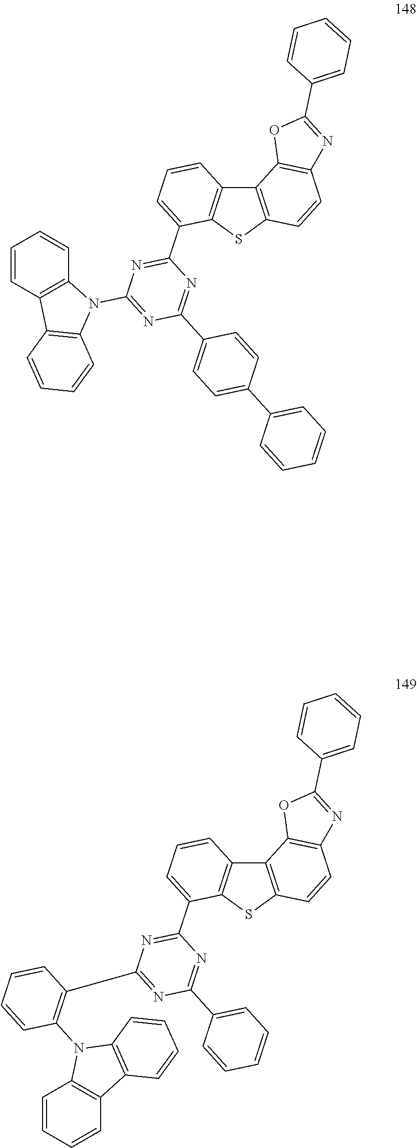 [SMILES: c1ccc(-c2ccc(-c3nc(-c4cccc5c4sc4ccc6nc(-c7ccccc7)oc6c45)nc(-n4c5ccccc5c5ccccc54)n3)cc2)cc1 ; c1ccc(-c2nc(-c3ccccc3-n3c4ccccc4c4ccccc43)nc(-c3cccc4c3sc3ccc5nc(-c6ccccc6)oc5c34)n2)cc1]

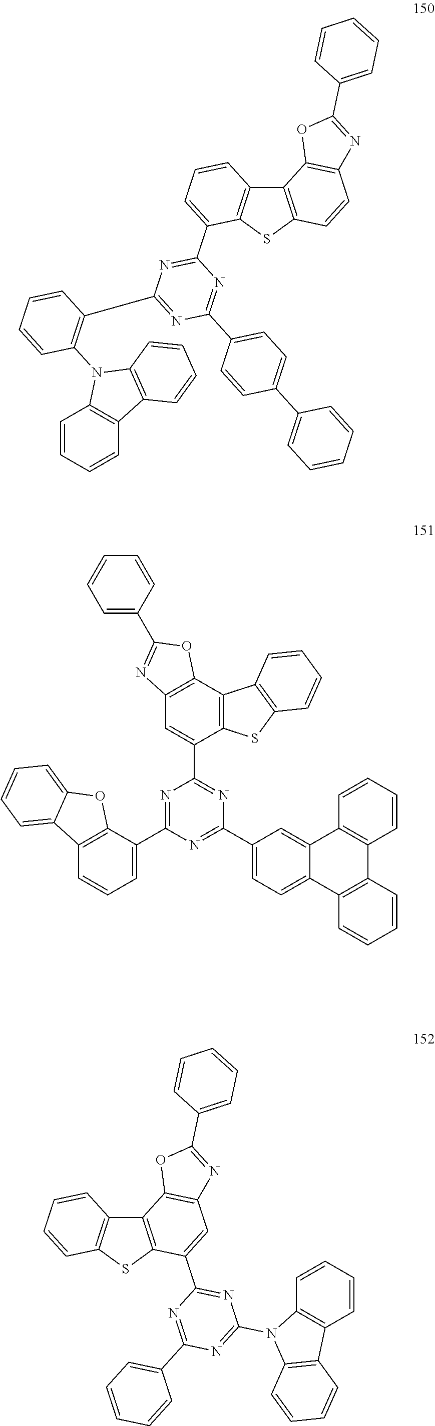 [SMILES: c1ccc(-c2ccc(-c3nc(-c4ccccc4-n4c5ccccc5c5ccccc54)nc(-c4cccc5c4sc4ccc6nc(-c7ccccc7)oc6c45)n3)cc2)cc1 ; c1ccc(-c2nc3cc(-c4nc(-c5ccc6c7ccccc7c7ccccc7c6c5)nc(-c5cccc6c5oc5ccccc56)n4)c4sc5ccccc5c4c3o2)cc1 ; c1ccc(-c2nc(-c3cc4nc(-c5ccccc5)oc4c4c3sc3ccccc34)nc(-n3c4ccccc4c4ccccc43)n2)cc1]

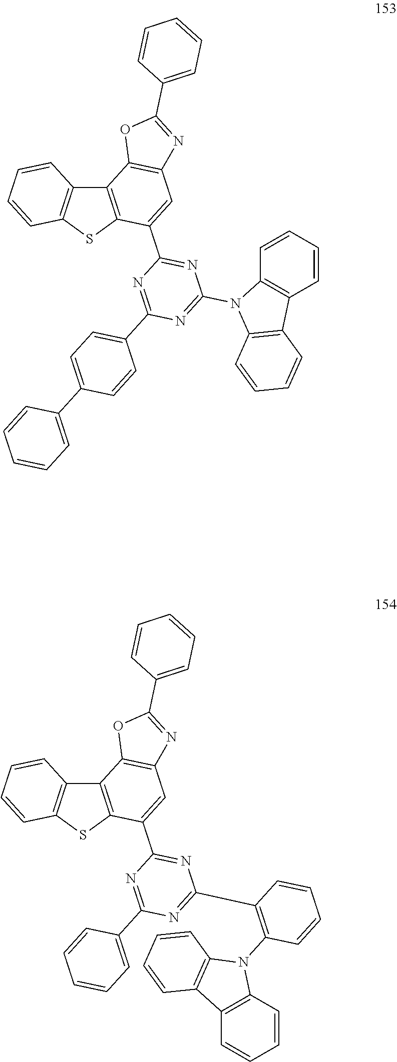 [SMILES: c1ccc(-c2ccc(-c3nc(-c4cc5nc(-c6ccccc6)oc5c5c4sc4ccccc45)nc(-n4c5ccccc5c5ccccc54)n3)cc2)cc1 ; c1ccc(-c2nc(-c3ccccc3-n3c4ccccc4c4ccccc43)nc(-c3cc4nc(-c5ccccc5)oc4c4c3sc3ccccc34)n2)cc1]

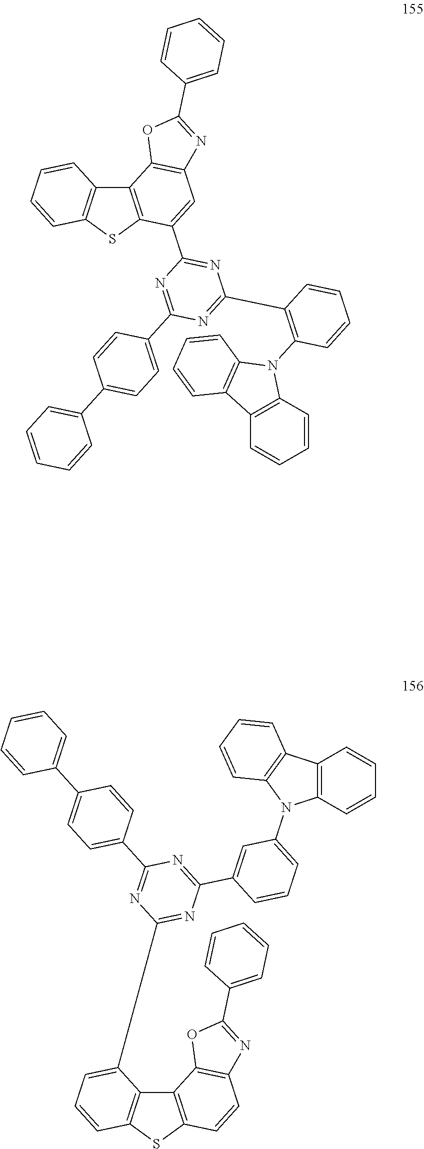 [SMILES: c1ccc(-c2ccc(-c3nc(-c4ccccc4-n4c5ccccc5c5ccccc54)nc(-c4cc5nc(-c6ccccc6)oc5c5c4sc4ccccc45)n3)cc2)cc1 ; c1ccc(-c2ccc(-c3nc(-c4cccc(-n5c6ccccc6c6ccccc65)c4)nc(-c4cccc5sc6ccc7nc(-c8ccccc8)oc7c6c45)n3)cc2)cc1]

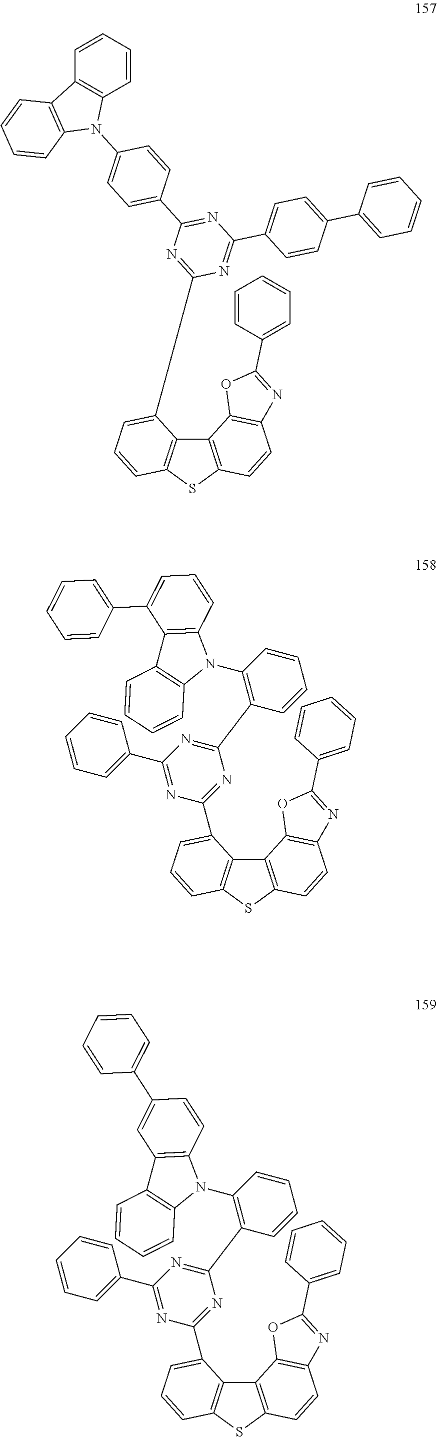 [SMILES: c1ccc(-c2ccc(-c3nc(-c4ccc(-n5c6ccccc6c6ccccc65)cc4)nc(-c4cccc5sc6ccc7nc(-c8ccccc8)oc7c6c45)n3)cc2)cc1 ; c1ccc(-c2nc(-c3ccccc3-n3c4ccccc4c4c(-c5ccccc5)cccc43)nc(-c3cccc4sc5ccc6nc(-c7ccccc7)oc6c5c34)n2)cc1 ; c1ccc(-c2ccc3c(c2)c2ccccc2n3-c2ccccc2-c2nc(-c3ccccc3)nc(-c3cccc4sc5ccc6nc(-c7ccccc7)oc6c5c34)n2)cc1]

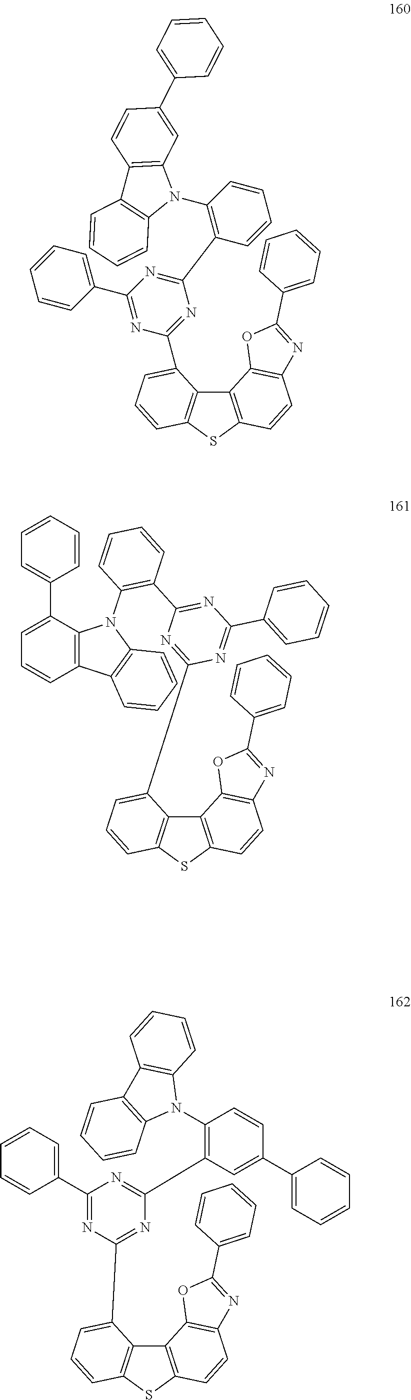 [SMILES: c1ccc(-c2ccc3c4ccccc4n(-c4ccccc4-c4nc(-c5ccccc5)nc(-c5cccc6sc7ccc8nc(-c9ccccc9)oc8c7c56)n4)c3c2)cc1 ; c1ccc(-c2nc(-c3ccccc3-n3c4ccccc4c4cccc(-c5ccccc5)c43)nc(-c3cccc4sc5ccc6nc(-c7ccccc7)oc6c5c34)n2)cc1 ; c1ccc(-c2ccc(-n3c4ccccc4c4ccccc43)c(-c3nc(-c4ccccc4)nc(-c4cccc5sc6ccc7nc(-c8ccccc8)oc7c6c45)n3)c2)cc1]

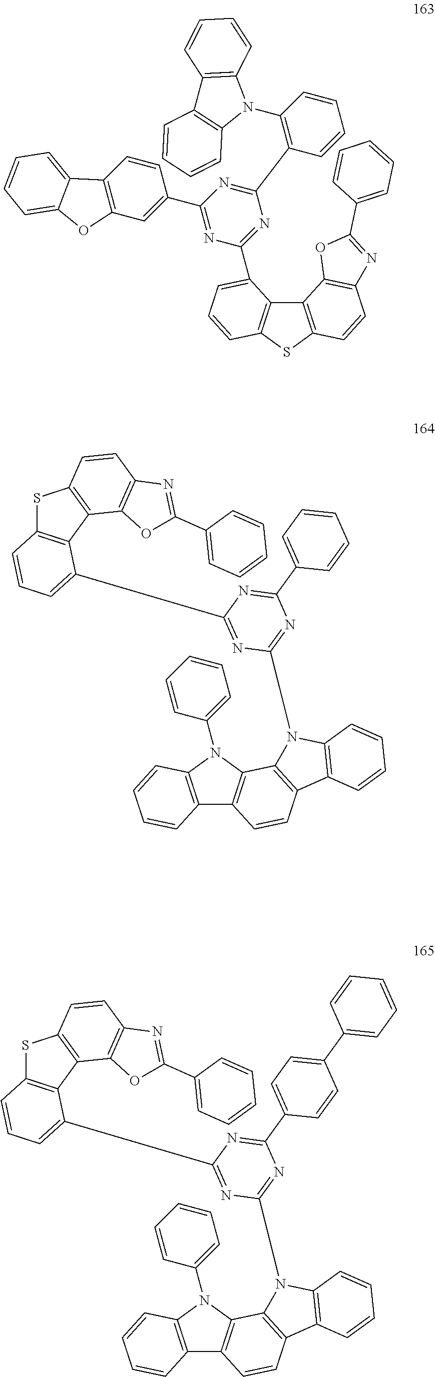 [SMILES: c1ccc(-c2nc3ccc4sc5cccc(-c6nc(-c7ccc8c(c7)oc7ccccc78)nc(-c7ccccc7-n7c8ccccc8c8ccccc87)n6)c5c4c3o2)cc1 ; c1ccc(-c2nc(-c3cccc4sc5ccc6nc(-c7ccccc7)oc6c5c34)nc(-n3c4ccccc4c4ccc5c6ccccc6n(-c6ccccc6)c5c43)n2)cc1 ; c1ccc(-c2ccc(-c3nc(-c4cccc5sc6ccc7nc(-c8ccccc8)oc7c6c45)nc(-n4c5ccccc5c5ccc6c7ccccc7n(-c7ccccc7)c6c54)n3)cc2)cc1]

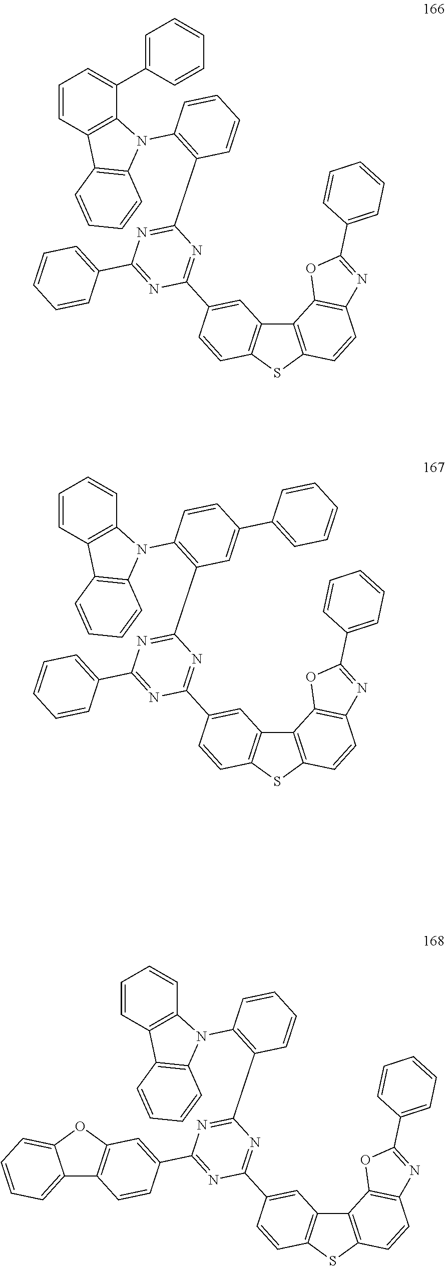 [SMILES: c1ccc(-c2nc(-c3ccc4sc5ccc6nc(-c7ccccc7)oc6c5c4c3)nc(-c3ccccc3-n3c4ccccc4c4cccc(-c5ccccc5)c43)n2)cc1 ; c1ccc(-c2ccc(-n3c4ccccc4c4ccccc43)c(-c3nc(-c4ccccc4)nc(-c4ccc5sc6ccc7nc(-c8ccccc8)oc7c6c5c4)n3)c2)cc1 ; c1ccc(-c2nc3ccc4sc5ccc(-c6nc(-c7ccc8c(c7)oc7ccccc78)nc(-c7ccccc7-n7c8ccccc8c8ccccc87)n6)cc5c4c3o2)cc1]

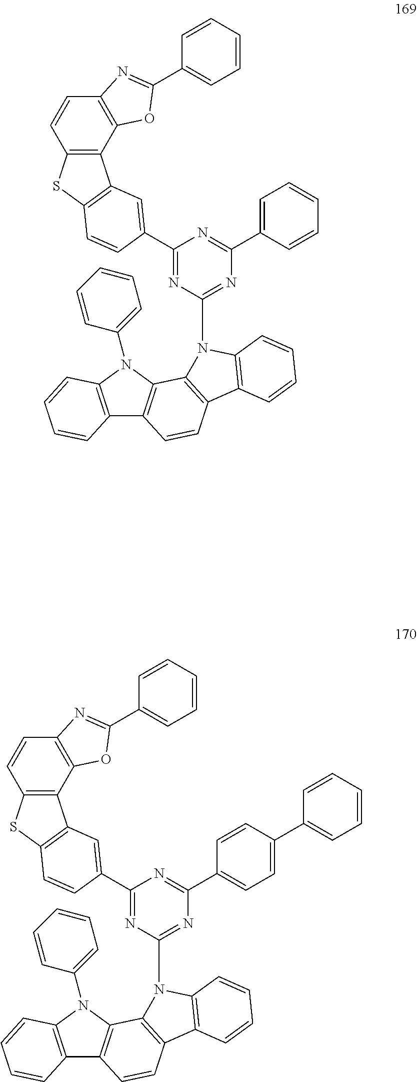 [SMILES: c1ccc(-c2nc(-c3ccc4sc5ccc6nc(-c7ccccc7)oc6c5c4c3)nc(-n3c4ccccc4c4ccc5c6ccccc6n(-c6ccccc6)c5c43)n2)cc1 ; c1ccc(-c2ccc(-c3nc(-c4ccc5sc6ccc7nc(-c8ccccc8)oc7c6c5c4)nc(-n4c5ccccc5c5ccc6c7ccccc7n(-c7ccccc7)c6c54)n3)cc2)cc1]

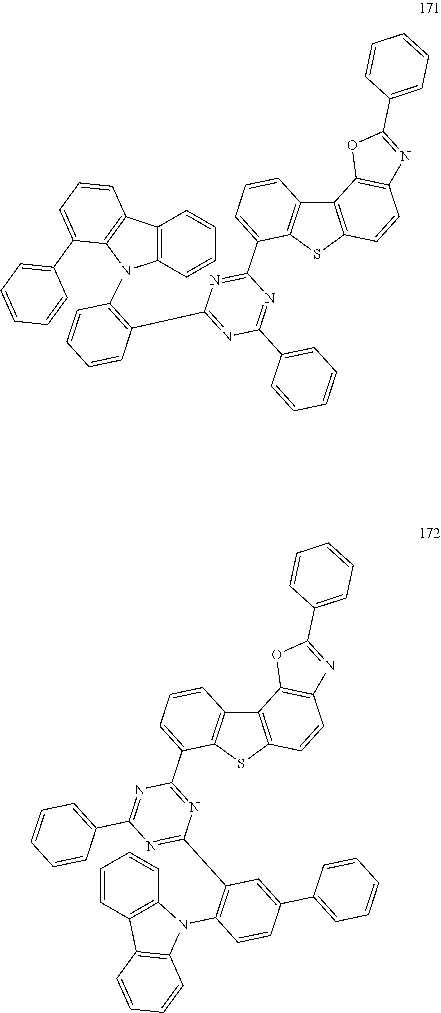 [SMILES: c1ccc(-c2nc(-c3ccccc3-n3c4ccccc4c4cccc(-c5ccccc5)c43)nc(-c3cccc4c3sc3ccc5nc(-c6ccccc6)oc5c34)n2)cc1 ; c1ccc(-c2ccc(-n3c4ccccc4c4ccccc43)c(-c3nc(-c4ccccc4)nc(-c4cccc5c4sc4ccc6nc(-c7ccccc7)oc6c45)n3)c2)cc1]

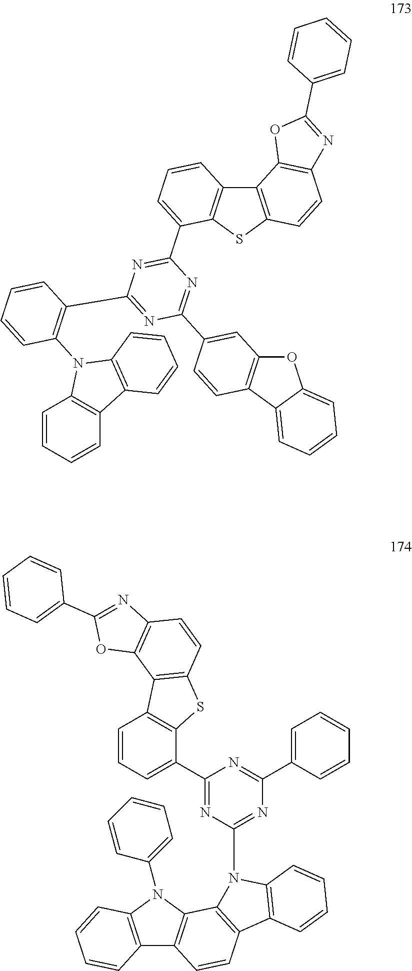 [SMILES: c1ccc(-c2nc3ccc4sc5c(-c6nc(-c7ccc8c(c7)oc7ccccc78)nc(-c7ccccc7-n7c8ccccc8c8ccccc87)n6)cccc5c4c3o2)cc1 ; c1ccc(-c2nc(-c3cccc4c3sc3ccc5nc(-c6ccccc6)oc5c34)nc(-n3c4ccccc4c4ccc5c6ccccc6n(-c6ccccc6)c5c43)n2)cc1]

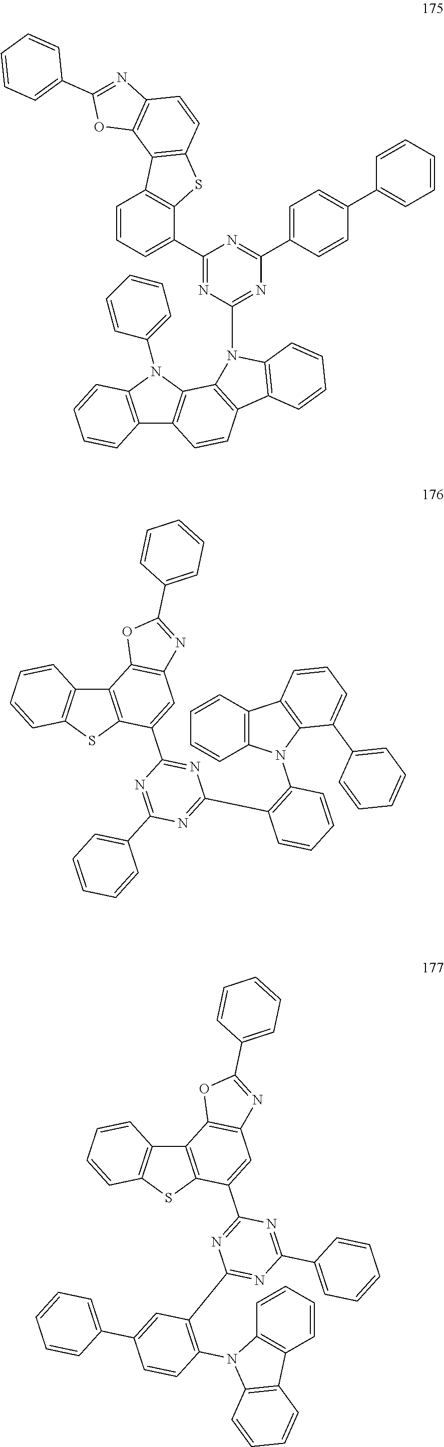 [SMILES: c1ccc(-c2ccc(-c3nc(-c4cccc5c4sc4ccc6nc(-c7ccccc7)oc6c45)nc(-n4c5ccccc5c5ccc6c7ccccc7n(-c7ccccc7)c6c54)n3)cc2)cc1 ; c1ccc(-c2nc(-c3ccccc3-n3c4ccccc4c4cccc(-c5ccccc5)c43)nc(-c3cc4nc(-c5ccccc5)oc4c4c3sc3ccccc34)n2)cc1 ; c1ccc(-c2ccc(-n3c4ccccc4c4ccccc43)c(-c3nc(-c4ccccc4)nc(-c4cc5nc(-c6ccccc6)oc5c5c4sc4ccccc45)n3)c2)cc1]

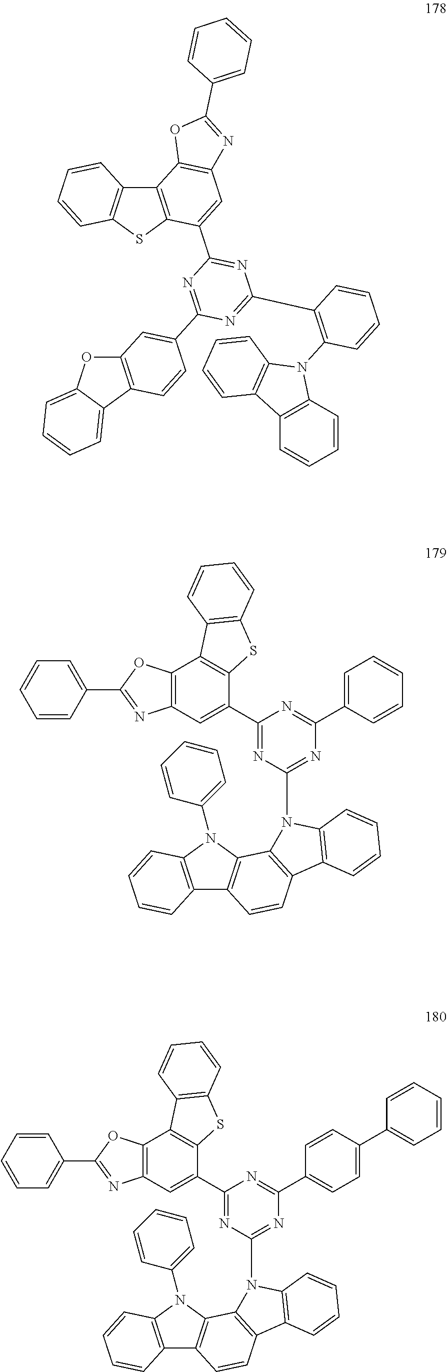 [SMILES: c1ccc(-c2nc3cc(-c4nc(-c5ccc6c(c5)oc5ccccc56)nc(-c5ccccc5-n5c6ccccc6c6ccccc65)n4)c4sc5ccccc5c4c3o2)cc1 ; c1ccc(-c2nc(-c3cc4nc(-c5ccccc5)oc4c4c3sc3ccccc34)nc(-n3c4ccccc4c4ccc5c6ccccc6n(-c6ccccc6)c5c43)n2)cc1 ; c1ccc(-c2ccc(-c3nc(-c4cc5nc(-c6ccccc6)oc5c5c4sc4ccccc45)nc(-n4c5ccccc5c5ccc6c7ccccc7n(-c7ccccc7)c6c54)n3)cc2)cc1]

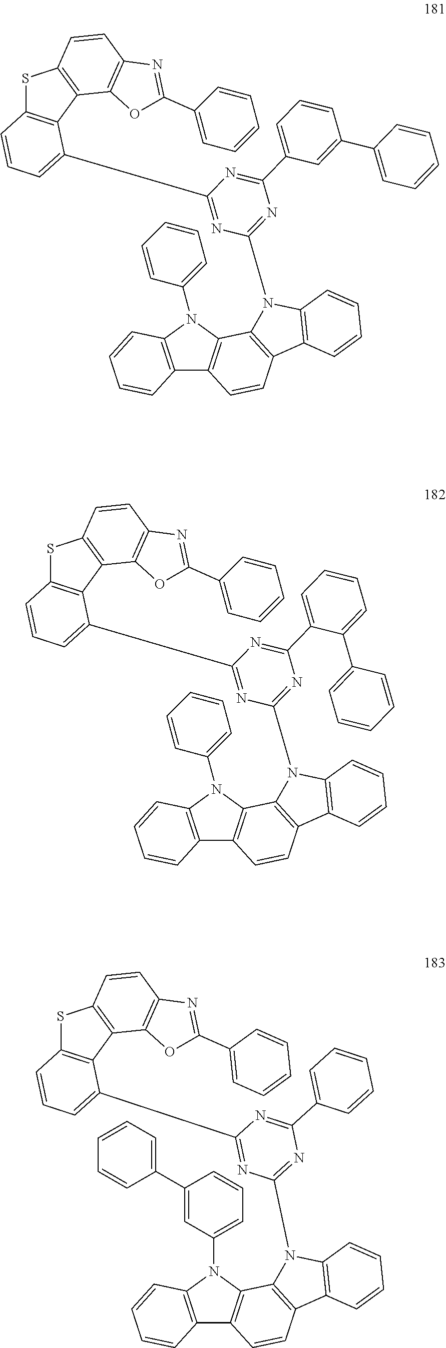 [SMILES: c1ccc(-c2cccc(-c3nc(-c4cccc5sc6ccc7nc(-c8ccccc8)oc7c6c45)nc(-n4c5ccccc5c5ccc6c7ccccc7n(-c7ccccc7)c6c54)n3)c2)cc1 ; c1ccc(-c2nc3ccc4sc5cccc(-c6nc(-c7ccccc7-c7ccccc7)nc(-n7c8ccccc8c8ccc9c%10ccccc%10n(-c%10ccccc%10)c9c87)n6)c5c4c3o2)cc1 ; c1ccc(-c2cccc(-n3c4ccccc4c4ccc5c6ccccc6n(-c6nc(-c7ccccc7)nc(-c7cccc8sc9ccc%10nc(-c%11ccccc%11)oc%10c9c78)n6)c5c43)c2)cc1]

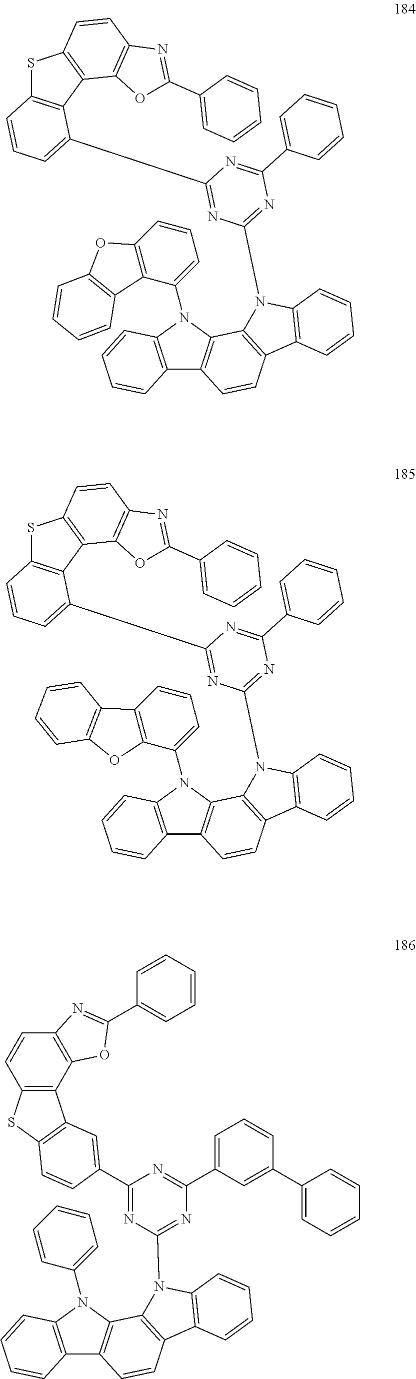 [SMILES: c1ccc(-c2nc(-c3cccc4sc5ccc6nc(-c7ccccc7)oc6c5c34)nc(-n3c4ccccc4c4ccc5c6ccccc6n(-c6cccc7oc8ccccc8c67)c5c43)n2)cc1 ; c1ccc(-c2nc(-c3cccc4sc5ccc6nc(-c7ccccc7)oc6c5c34)nc(-n3c4ccccc4c4ccc5c6ccccc6n(-c6cccc7c6oc6ccccc67)c5c43)n2)cc1 ; c1ccc(-c2cccc(-c3nc(-c4ccc5sc6ccc7nc(-c8ccccc8)oc7c6c5c4)nc(-n4c5ccccc5c5ccc6c7ccccc7n(-c7ccccc7)c6c54)n3)c2)cc1]

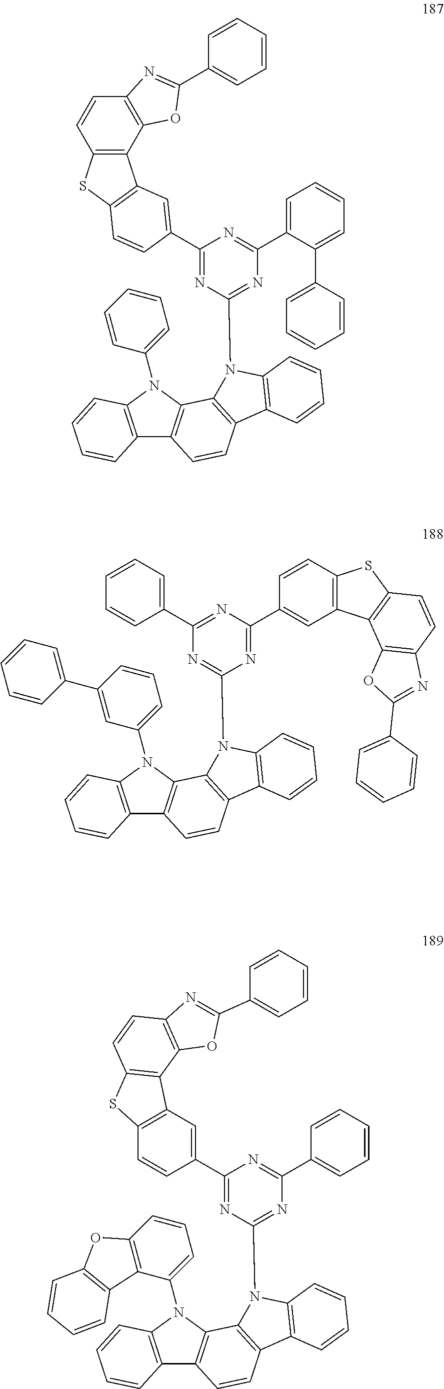 [SMILES: c1ccc(-c2nc3ccc4sc5ccc(-c6nc(-c7ccccc7-c7ccccc7)nc(-n7c8ccccc8c8ccc9c%10ccccc%10n(-c%10ccccc%10)c9c87)n6)cc5c4c3o2)cc1 ; c1ccc(-c2cccc(-n3c4ccccc4c4ccc5c6ccccc6n(-c6nc(-c7ccccc7)nc(-c7ccc8sc9ccc%10nc(-c%11ccccc%11)oc%10c9c8c7)n6)c5c43)c2)cc1 ; c1ccc(-c2nc(-c3ccc4sc5ccc6nc(-c7ccccc7)oc6c5c4c3)nc(-n3c4ccccc4c4ccc5c6ccccc6n(-c6cccc7oc8ccccc8c67)c5c43)n2)cc1]

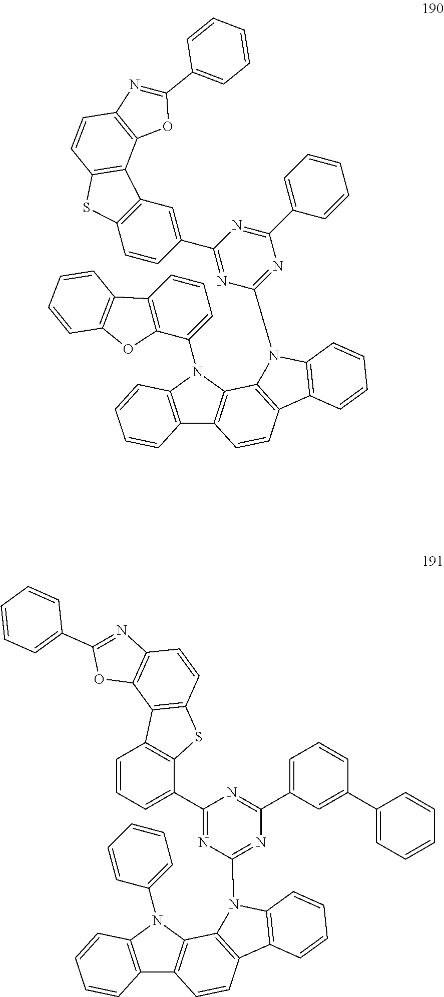 [SMILES: c1ccc(-c2nc(-c3ccc4sc5ccc6nc(-c7ccccc7)oc6c5c4c3)nc(-n3c4ccccc4c4ccc5c6ccccc6n(-c6cccc7c6oc6ccccc67)c5c43)n2)cc1 ; c1ccc(-c2cccc(-c3nc(-c4cccc5c4sc4ccc6nc(-c7ccccc7)oc6c45)nc(-n4c5ccccc5c5ccc6c7ccccc7n(-c7ccccc7)c6c54)n3)c2)cc1]

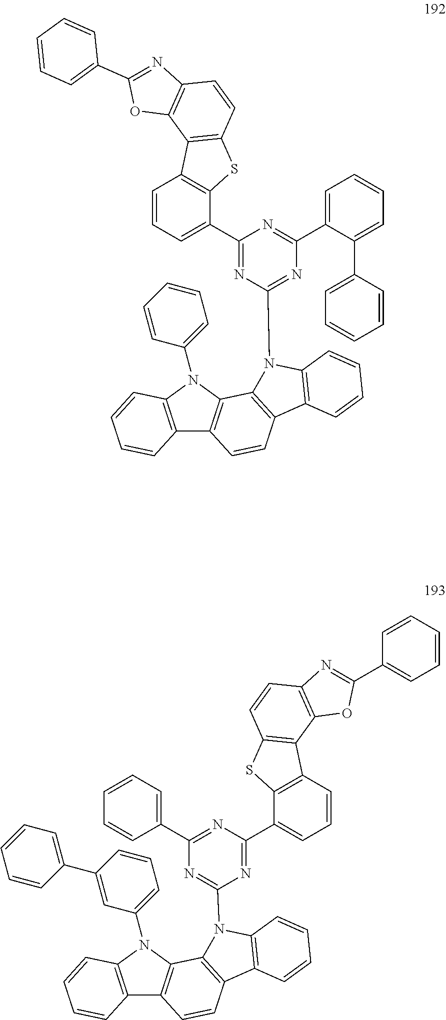 [SMILES: c1ccc(-c2nc3ccc4sc5c(-c6nc(-c7ccccc7-c7ccccc7)nc(-n7c8ccccc8c8ccc9c%10ccccc%10n(-c%10ccccc%10)c9c87)n6)cccc5c4c3o2)cc1 ; c1ccc(-c2cccc(-n3c4ccccc4c4ccc5c6ccccc6n(-c6nc(-c7ccccc7)nc(-c7cccc8c7sc7ccc9nc(-c%10ccccc%10)oc9c78)n6)c5c43)c2)cc1]

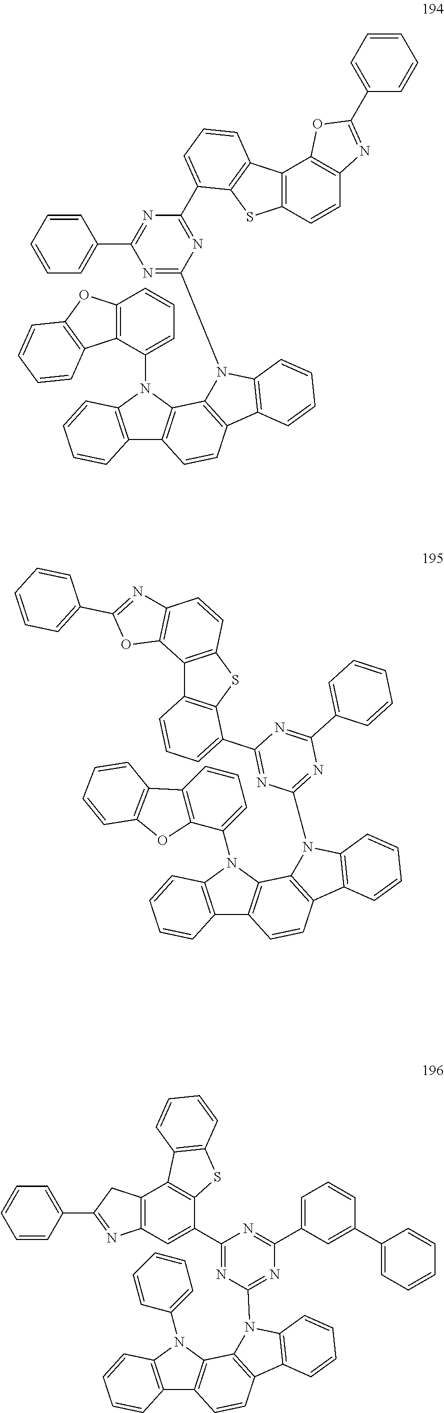 [SMILES: c1ccc(-c2nc(-c3cccc4c3sc3ccc5nc(-c6ccccc6)oc5c34)nc(-n3c4ccccc4c4ccc5c6ccccc6n(-c6cccc7oc8ccccc8c67)c5c43)n2)cc1 ; c1ccc(-c2nc(-c3cccc4c3sc3ccc5nc(-c6ccccc6)oc5c34)nc(-n3c4ccccc4c4ccc5c6ccccc6n(-c6cccc7c6oc6ccccc67)c5c43)n2)cc1 ; c1ccc(C2=Nc3cc(-c4nc(-c5cccc(-c6ccccc6)c5)nc(-n5c6ccccc6c6ccc7c8ccccc8n(-c8ccccc8)c7c65)n4)c4sc5ccccc5c4c3C2)cc1]

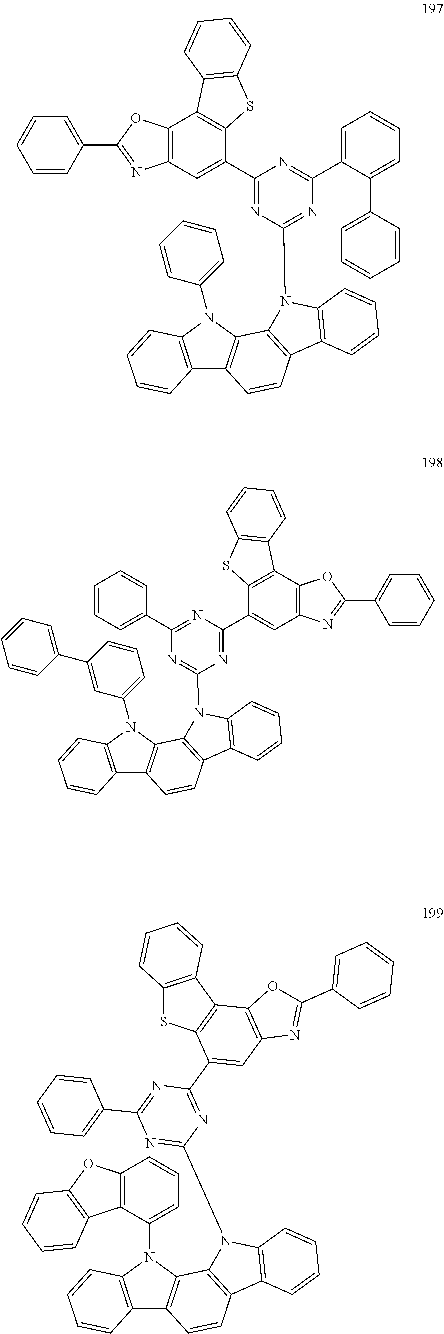 [SMILES: c1ccc(-c2nc3cc(-c4nc(-c5ccccc5-c5ccccc5)nc(-n5c6ccccc6c6ccc7c8ccccc8n(-c8ccccc8)c7c65)n4)c4sc5ccccc5c4c3o2)cc1 ; c1ccc(-c2cccc(-n3c4ccccc4c4ccc5c6ccccc6n(-c6nc(-c7ccccc7)nc(-c7cc8nc(-c9ccccc9)oc8c8c7sc7ccccc78)n6)c5c43)c2)cc1 ; c1ccc(-c2nc(-c3cc4nc(-c5ccccc5)oc4c4c3sc3ccccc34)nc(-n3c4ccccc4c4ccc5c6ccccc6n(-c6cccc7oc8ccccc8c67)c5c43)n2)cc1]

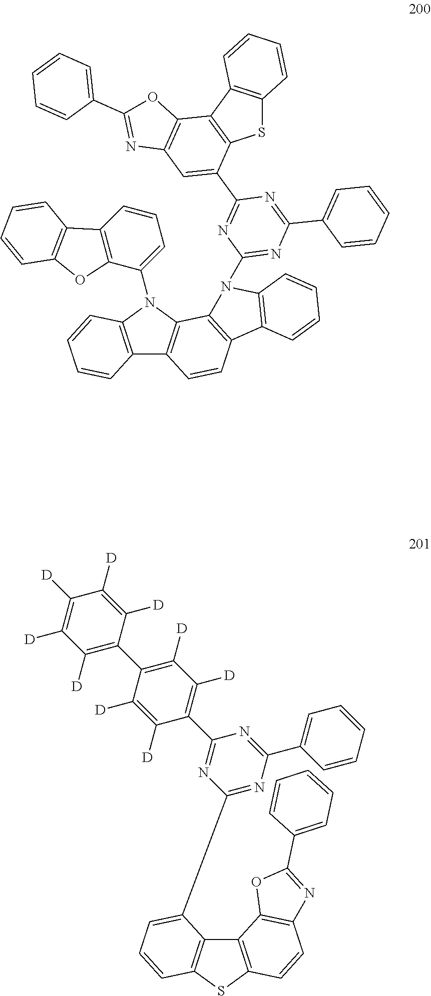 [SMILES: c1ccc(-c2nc(-c3cc4nc(-c5ccccc5)oc4c4c3sc3ccccc34)nc(-n3c4ccccc4c4ccc5c6ccccc6n(-c6cccc7c6oc6ccccc67)c5c43)n2)cc1 ; [2H]c1c([2H])c([2H])c(-c2c([2H])c([2H])c(-c3nc(-c4ccccc4)nc(-c4cccc5sc6ccc7nc(-c8ccccc8)oc7c6c45)n3)c([2H])c2[2H])c([2H])c1[2H]]

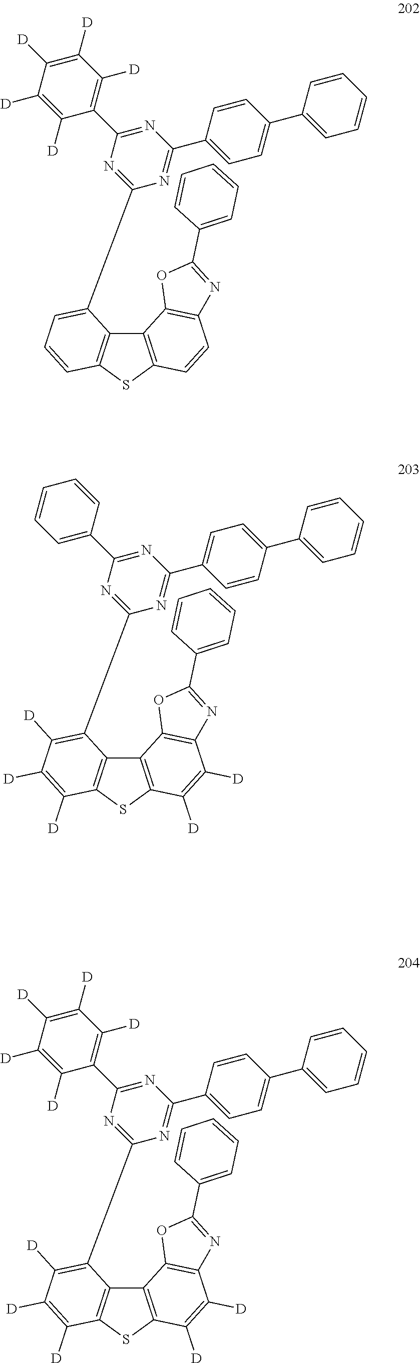 [SMILES: [2H]c1c([2H])c([2H])c(-c2nc(-c3ccc(-c4ccccc4)cc3)nc(-c3cccc4sc5ccc6nc(-c7ccccc7)oc6c5c34)n2)c([2H])c1[2H] ; [2H]c1c([2H])c(-c2nc(-c3ccccc3)nc(-c3ccc(-c4ccccc4)cc3)n2)c2c(sc3c([2H])c([2H])c4nc(-c5ccccc5)oc4c32)c1[2H] ; [2H]c1c([2H])c([2H])c(-c2nc(-c3ccc(-c4ccccc4)cc3)nc(-c3c([2H])c([2H])c([2H])c4sc5c([2H])c([2H])c6nc(-c7ccccc7)oc6c5c34)n2)c([2H])c1[2H]]

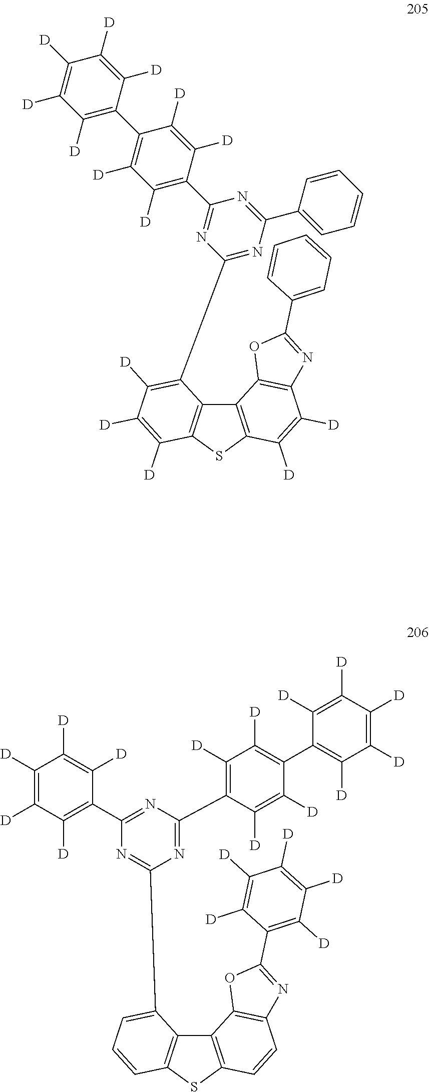 [SMILES: [2H]c1c([2H])c([2H])c(-c2c([2H])c([2H])c(-c3nc(-c4ccccc4)nc(-c4c([2H])c([2H])c([2H])c5sc6c([2H])c([2H])c7nc(-c8ccccc8)oc7c6c45)n3)c([2H])c2[2H])c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3cccc4sc5ccc6nc(-c7c([2H])c([2H])c([2H])c([2H])c7[2H])oc6c5c34)n2)c([2H])c1[2H]]

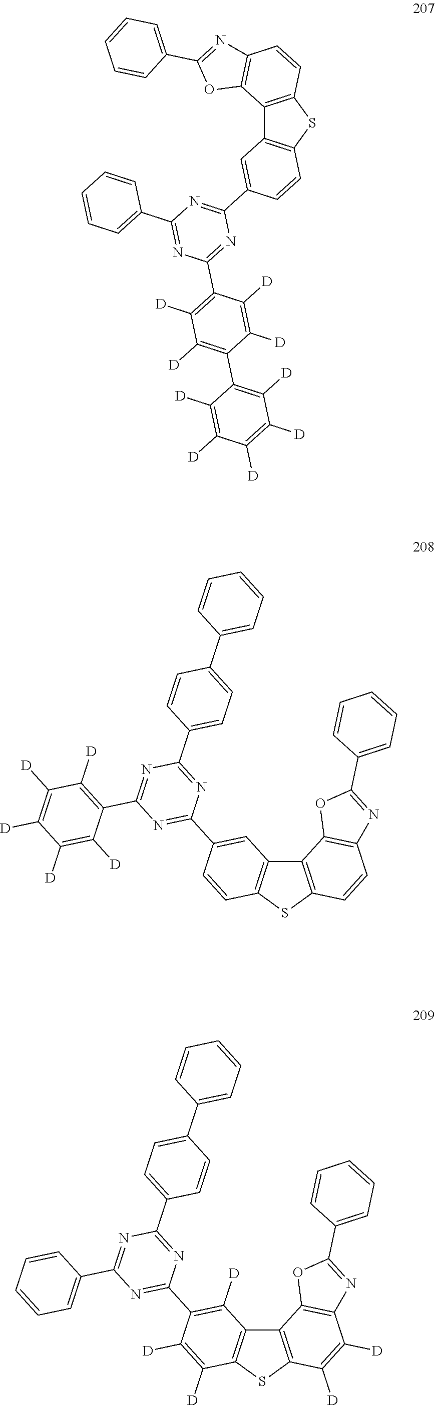 [SMILES: [2H]c1c([2H])c([2H])c(-c2c([2H])c([2H])c(-c3nc(-c4ccccc4)nc(-c4ccc5sc6ccc7nc(-c8ccccc8)oc7c6c5c4)n3)c([2H])c2[2H])c([2H])c1[2H] ; [2H]c1c([2H])c([2H])c(-c2nc(-c3ccc(-c4ccccc4)cc3)nc(-c3ccc4sc5ccc6nc(-c7ccccc7)oc6c5c4c3)n2)c([2H])c1[2H] ; [2H]c1c(-c2nc(-c3ccccc3)nc(-c3ccc(-c4ccccc4)cc3)n2)c([2H])c2c(sc3c([2H])c([2H])c4nc(-c5ccccc5)oc4c32)c1[2H]]

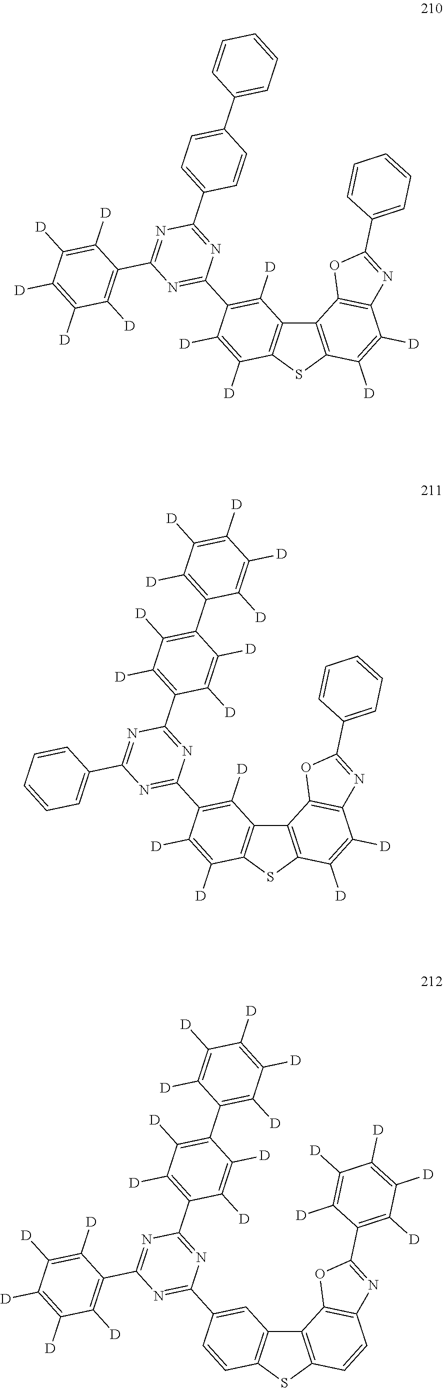 [SMILES: [2H]c1c([2H])c([2H])c(-c2nc(-c3ccc(-c4ccccc4)cc3)nc(-c3c([2H])c([2H])c4sc5c([2H])c([2H])c6nc(-c7ccccc7)oc6c5c4c3[2H])n2)c([2H])c1[2H] ; [2H]c1c([2H])c([2H])c(-c2c([2H])c([2H])c(-c3nc(-c4ccccc4)nc(-c4c([2H])c([2H])c5sc6c([2H])c([2H])c7nc(-c8ccccc8)oc7c6c5c4[2H])n3)c([2H])c2[2H])c([2H])c1[2H] ; [2H]c1c([2H])c([2H])c(-c2nc(-c3ccc4sc5ccc6nc(-c7c([2H])c([2H])c([2H])c([2H])c7[2H])oc6c5c4c3)nc(-c3c([2H])c([2H])c(-c4c([2H])c([2H])c([2H])c([2H])c4[2H])c([2H])c3[2H])n2)c([2H])c1[2H]]

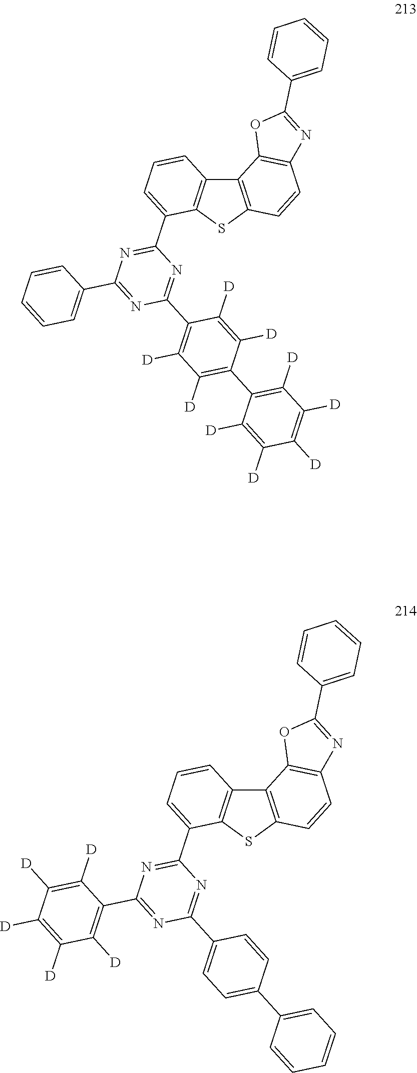 [SMILES: [2H]c1c([2H])c([2H])c(-c2c([2H])c([2H])c(-c3nc(-c4ccccc4)nc(-c4cccc5c4sc4ccc6nc(-c7ccccc7)oc6c45)n3)c([2H])c2[2H])c([2H])c1[2H] ; [2H]c1c([2H])c([2H])c(-c2nc(-c3ccc(-c4ccccc4)cc3)nc(-c3cccc4c3sc3ccc5nc(-c6ccccc6)oc5c34)n2)c([2H])c1[2H]]

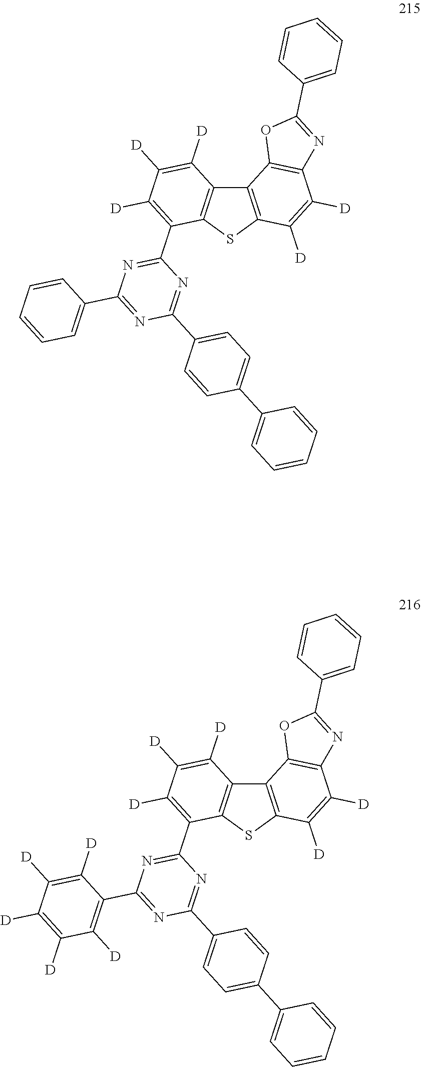 [SMILES: [2H]c1c([2H])c([2H])c2c(sc3c([2H])c([2H])c4nc(-c5ccccc5)oc4c32)c1-c1nc(-c2ccccc2)nc(-c2ccc(-c3ccccc3)cc2)n1 ; [2H]c1c([2H])c([2H])c(-c2nc(-c3ccc(-c4ccccc4)cc3)nc(-c3c([2H])c([2H])c([2H])c4c3sc3c([2H])c([2H])c5nc(-c6ccccc6)oc5c34)n2)c([2H])c1[2H]]

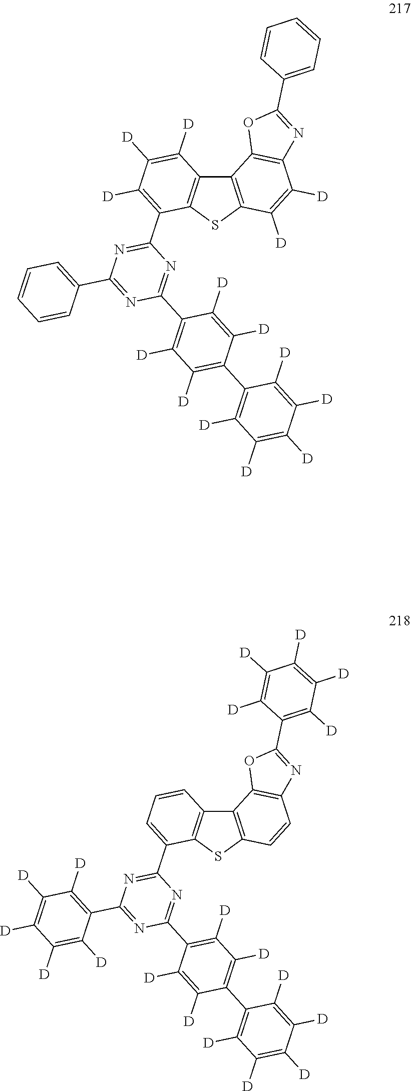 [SMILES: [2H]c1c([2H])c([2H])c(-c2c([2H])c([2H])c(-c3nc(-c4ccccc4)nc(-c4c([2H])c([2H])c([2H])c5c4sc4c([2H])c([2H])c6nc(-c7ccccc7)oc6c45)n3)c([2H])c2[2H])c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3cccc4c3sc3ccc5nc(-c6c([2H])c([2H])c([2H])c([2H])c6[2H])oc5c34)n2)c([2H])c1[2H]]

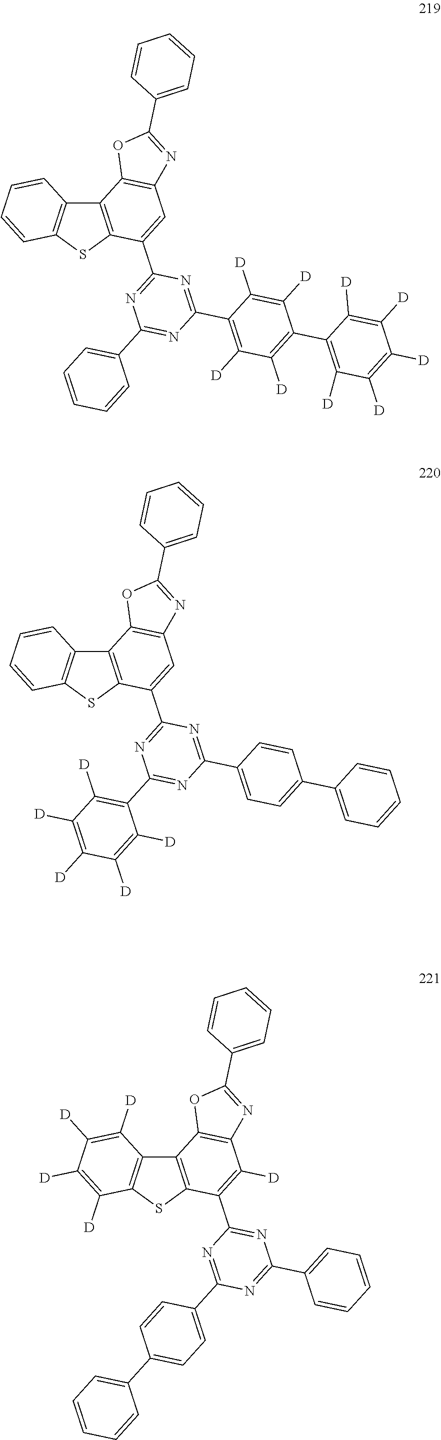 [SMILES: [2H]c1c([2H])c([2H])c(-c2c([2H])c([2H])c(-c3nc(-c4ccccc4)nc(-c4cc5nc(-c6ccccc6)oc5c5c4sc4ccccc45)n3)c([2H])c2[2H])c([2H])c1[2H] ; [2H]c1c([2H])c([2H])c(-c2nc(-c3ccc(-c4ccccc4)cc3)nc(-c3cc4nc(-c5ccccc5)oc4c4c3sc3ccccc34)n2)c([2H])c1[2H] ; [2H]c1c([2H])c([2H])c2c(sc3c(-c4nc(-c5ccccc5)nc(-c5ccc(-c6ccccc6)cc5)n4)c([2H])c4nc(-c5ccccc5)oc4c32)c1[2H]]

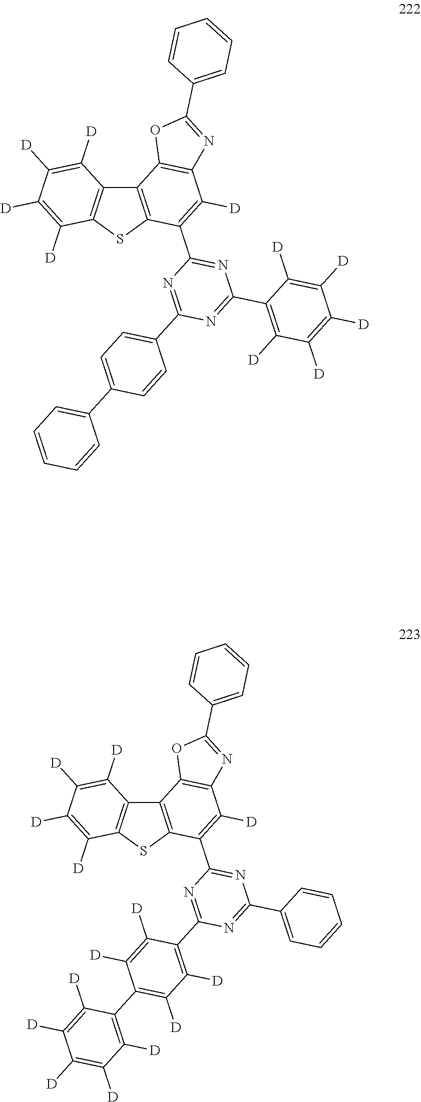 [SMILES: [2H]c1c([2H])c([2H])c(-c2nc(-c3ccc(-c4ccccc4)cc3)nc(-c3c([2H])c4nc(-c5ccccc5)oc4c4c3sc3c([2H])c([2H])c([2H])c([2H])c34)n2)c([2H])c1[2H] ; [2H]c1c([2H])c([2H])c(-c2c([2H])c([2H])c(-c3nc(-c4ccccc4)nc(-c4c([2H])c5nc(-c6ccccc6)oc5c5c4sc4c([2H])c([2H])c([2H])c([2H])c45)n3)c([2H])c2[2H])c([2H])c1[2H]]

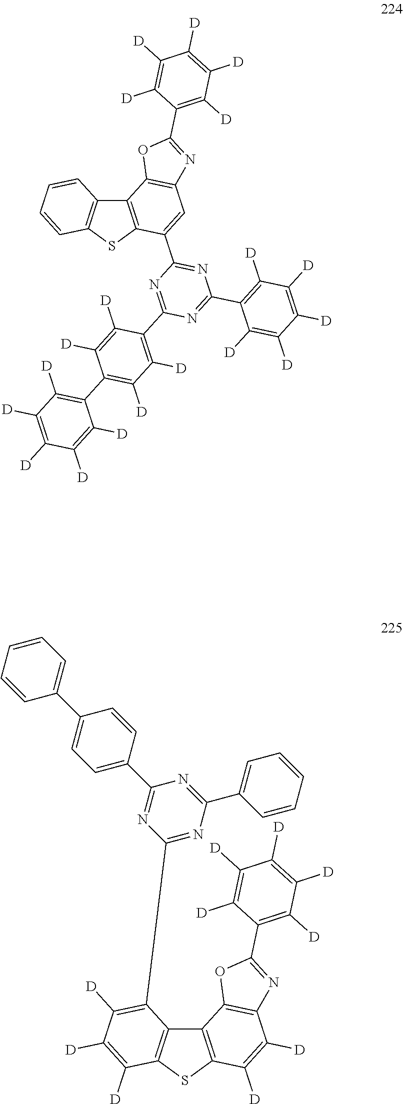 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3cc4nc(-c5c([2H])c([2H])c([2H])c([2H])c5[2H])oc4c4c3sc3ccccc34)n2)c([2H])c1[2H] ; [2H]c1c([2H])c([2H])c(-c2nc3c([2H])c([2H])c4sc5c([2H])c([2H])c([2H])c(-c6nc(-c7ccccc7)nc(-c7ccc(-c8ccccc8)cc7)n6)c5c4c3o2)c([2H])c1[2H]]

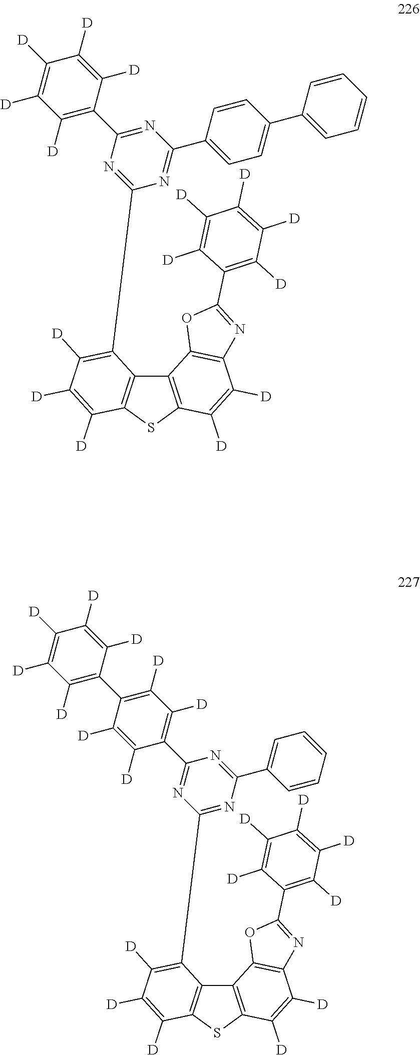 [SMILES: [2H]c1c([2H])c([2H])c(-c2nc(-c3ccc(-c4ccccc4)cc3)nc(-c3c([2H])c([2H])c([2H])c4sc5c([2H])c([2H])c6nc(-c7c([2H])c([2H])c([2H])c([2H])c7[2H])oc6c5c34)n2)c([2H])c1[2H] ; [2H]c1c([2H])c([2H])c(-c2nc3c([2H])c([2H])c4sc5c([2H])c([2H])c([2H])c(-c6nc(-c7ccccc7)nc(-c7c([2H])c([2H])c(-c8c([2H])c([2H])c([2H])c([2H])c8[2H])c([2H])c7[2H])n6)c5c4c3o2)c([2H])c1[2H]]

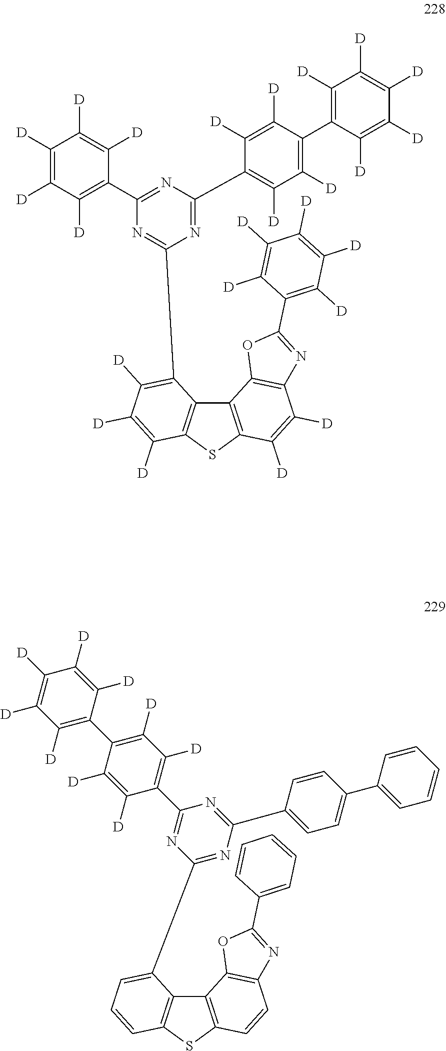 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c([2H])c4sc5c([2H])c([2H])c6nc(-c7c([2H])c([2H])c([2H])c([2H])c7[2H])oc6c5c34)n2)c([2H])c1[2H] ; [2H]c1c([2H])c([2H])c(-c2c([2H])c([2H])c(-c3nc(-c4ccc(-c5ccccc5)cc4)nc(-c4cccc5sc6ccc7nc(-c8ccccc8)oc7c6c45)n3)c([2H])c2[2H])c([2H])c1[2H]]

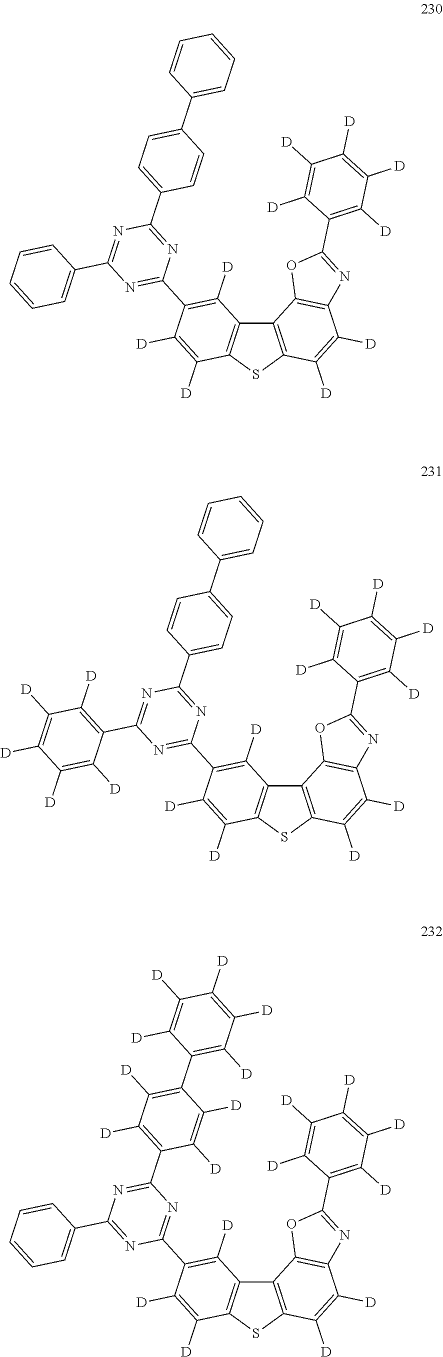 [SMILES: [2H]c1c([2H])c([2H])c(-c2nc3c([2H])c([2H])c4sc5c([2H])c([2H])c(-c6nc(-c7ccccc7)nc(-c7ccc(-c8ccccc8)cc7)n6)c([2H])c5c4c3o2)c([2H])c1[2H] ; [2H]c1c([2H])c([2H])c(-c2nc(-c3ccc(-c4ccccc4)cc3)nc(-c3c([2H])c([2H])c4sc5c([2H])c([2H])c6nc(-c7c([2H])c([2H])c([2H])c([2H])c7[2H])oc6c5c4c3[2H])n2)c([2H])c1[2H] ; [2H]c1c([2H])c([2H])c(-c2nc3c([2H])c([2H])c4sc5c([2H])c([2H])c(-c6nc(-c7ccccc7)nc(-c7c([2H])c([2H])c(-c8c([2H])c([2H])c([2H])c([2H])c8[2H])c([2H])c7[2H])n6)c([2H])c5c4c3o2)c([2H])c1[2H]]

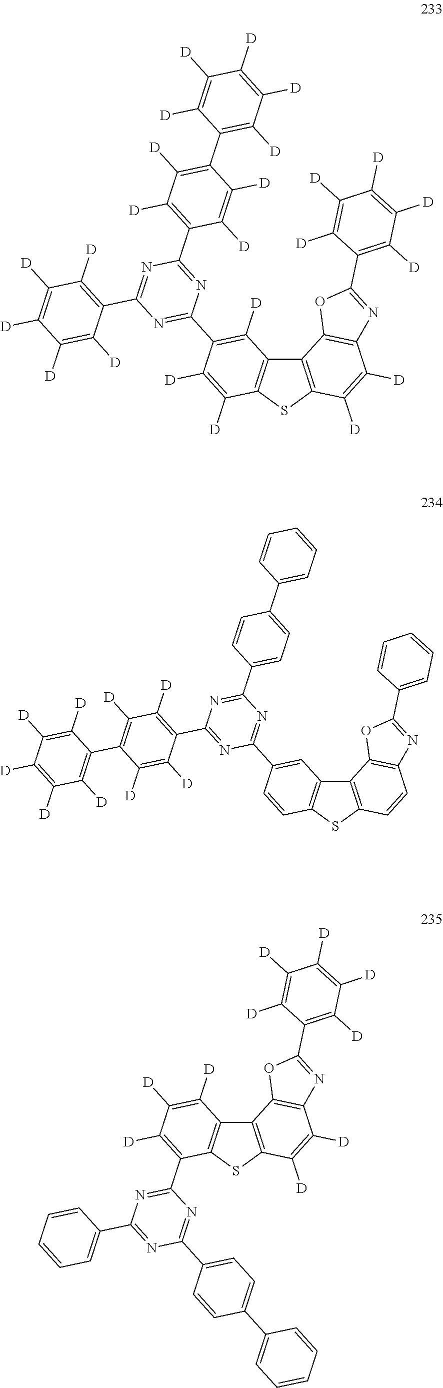 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c4sc5c([2H])c([2H])c6nc(-c7c([2H])c([2H])c([2H])c([2H])c7[2H])oc6c5c4c3[2H])n2)c([2H])c1[2H] ; [2H]c1c([2H])c([2H])c(-c2c([2H])c([2H])c(-c3nc(-c4ccc(-c5ccccc5)cc4)nc(-c4ccc5sc6ccc7nc(-c8ccccc8)oc7c6c5c4)n3)c([2H])c2[2H])c([2H])c1[2H] ; [2H]c1c([2H])c([2H])c(-c2nc3c([2H])c([2H])c4sc5c(-c6nc(-c7ccccc7)nc(-c7ccc(-c8ccccc8)cc7)n6)c([2H])c([2H])c([2H])c5c4c3o2)c([2H])c1[2H]]

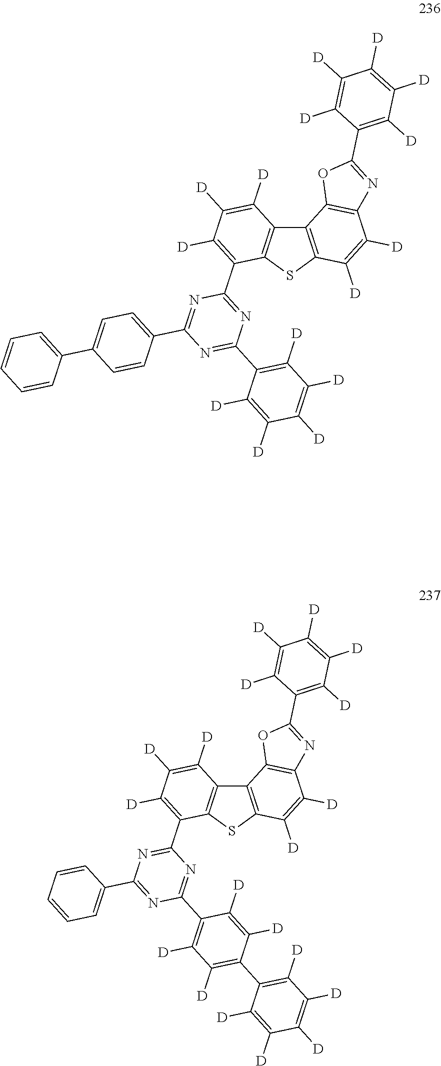 [SMILES: [2H]c1c([2H])c([2H])c(-c2nc(-c3ccc(-c4ccccc4)cc3)nc(-c3c([2H])c([2H])c([2H])c4c3sc3c([2H])c([2H])c5nc(-c6c([2H])c([2H])c([2H])c([2H])c6[2H])oc5c34)n2)c([2H])c1[2H] ; [2H]c1c([2H])c([2H])c(-c2nc3c([2H])c([2H])c4sc5c(-c6nc(-c7ccccc7)nc(-c7c([2H])c([2H])c(-c8c([2H])c([2H])c([2H])c([2H])c8[2H])c([2H])c7[2H])n6)c([2H])c([2H])c([2H])c5c4c3o2)c([2H])c1[2H]]

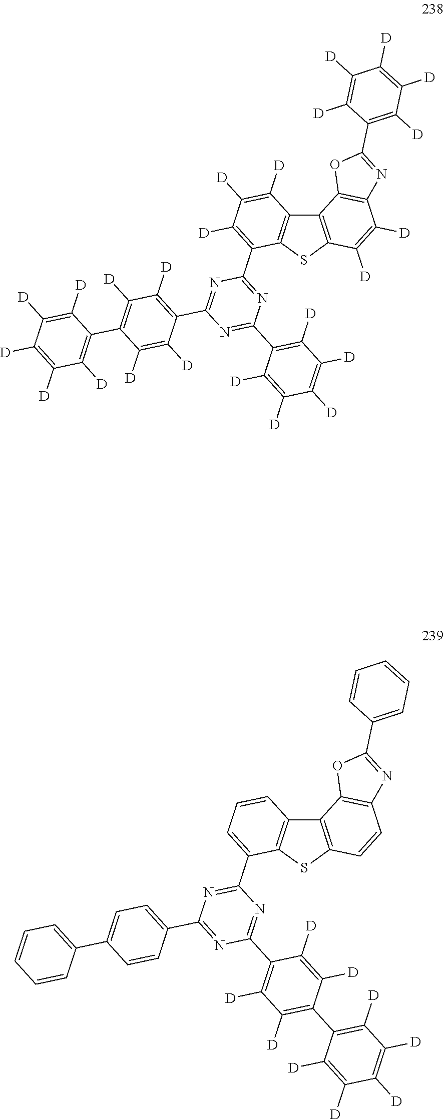 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c([2H])c4c3sc3c([2H])c([2H])c5nc(-c6c([2H])c([2H])c([2H])c([2H])c6[2H])oc5c34)n2)c([2H])c1[2H] ; [2H]c1c([2H])c([2H])c(-c2c([2H])c([2H])c(-c3nc(-c4ccc(-c5ccccc5)cc4)nc(-c4cccc5c4sc4ccc6nc(-c7ccccc7)oc6c45)n3)c([2H])c2[2H])c([2H])c1[2H]]

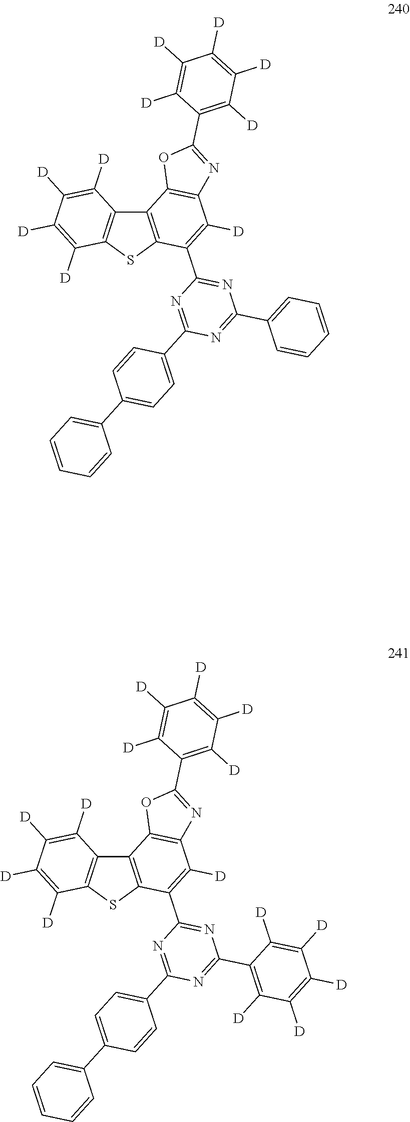 [SMILES: [2H]c1c([2H])c([2H])c(-c2nc3c([2H])c(-c4nc(-c5ccccc5)nc(-c5ccc(-c6ccccc6)cc5)n4)c4sc5c([2H])c([2H])c([2H])c([2H])c5c4c3o2)c([2H])c1[2H] ; [2H]c1c([2H])c([2H])c(-c2nc(-c3ccc(-c4ccccc4)cc3)nc(-c3c([2H])c4nc(-c5c([2H])c([2H])c([2H])c([2H])c5[2H])oc4c4c3sc3c([2H])c([2H])c([2H])c([2H])c34)n2)c([2H])c1[2H]]

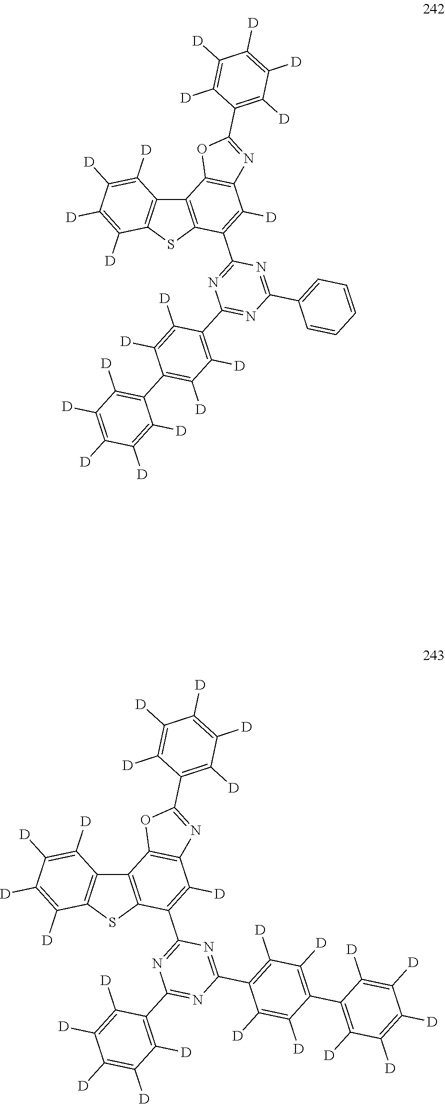 [SMILES: [2H]c1c([2H])c([2H])c(-c2nc3c([2H])c(-c4nc(-c5ccccc5)nc(-c5c([2H])c([2H])c(-c6c([2H])c([2H])c([2H])c([2H])c6[2H])c([2H])c5[2H])n4)c4sc5c([2H])c([2H])c([2H])c([2H])c5c4c3o2)c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c4nc(-c5c([2H])c([2H])c([2H])c([2H])c5[2H])oc4c4c3sc3c([2H])c([2H])c([2H])c([2H])c34)n2)c([2H])c1[2H]]

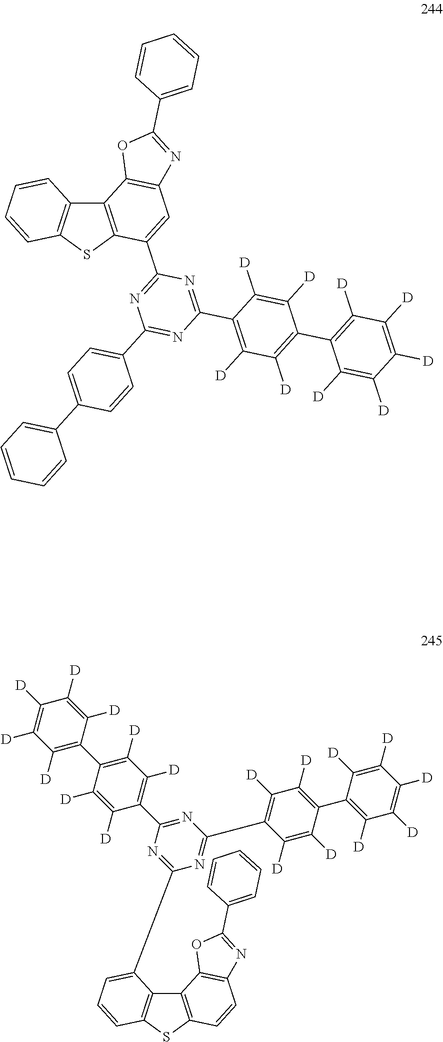 [SMILES: [2H]c1c([2H])c([2H])c(-c2c([2H])c([2H])c(-c3nc(-c4ccc(-c5ccccc5)cc4)nc(-c4cc5nc(-c6ccccc6)oc5c5c4sc4ccccc45)n3)c([2H])c2[2H])c([2H])c1[2H] ; [2H]c1c([2H])c([2H])c(-c2c([2H])c([2H])c(-c3nc(-c4c([2H])c([2H])c(-c5c([2H])c([2H])c([2H])c([2H])c5[2H])c([2H])c4[2H])nc(-c4cccc5sc6ccc7nc(-c8ccccc8)oc7c6c45)n3)c([2H])c2[2H])c([2H])c1[2H]]

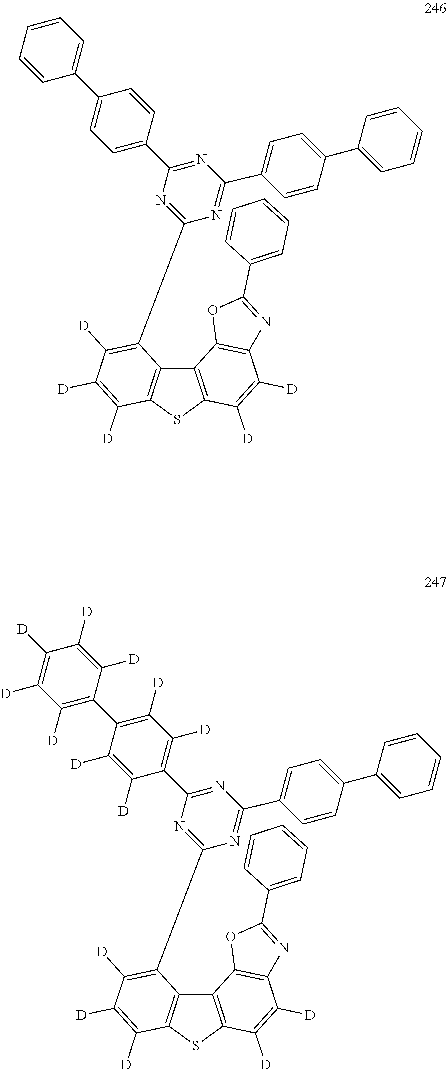 [SMILES: [2H]c1c([2H])c(-c2nc(-c3ccc(-c4ccccc4)cc3)nc(-c3ccc(-c4ccccc4)cc3)n2)c2c(sc3c([2H])c([2H])c4nc(-c5ccccc5)oc4c32)c1[2H] ; [2H]c1c([2H])c([2H])c(-c2c([2H])c([2H])c(-c3nc(-c4ccc(-c5ccccc5)cc4)nc(-c4c([2H])c([2H])c([2H])c5sc6c([2H])c([2H])c7nc(-c8ccccc8)oc7c6c45)n3)c([2H])c2[2H])c([2H])c1[2H]]

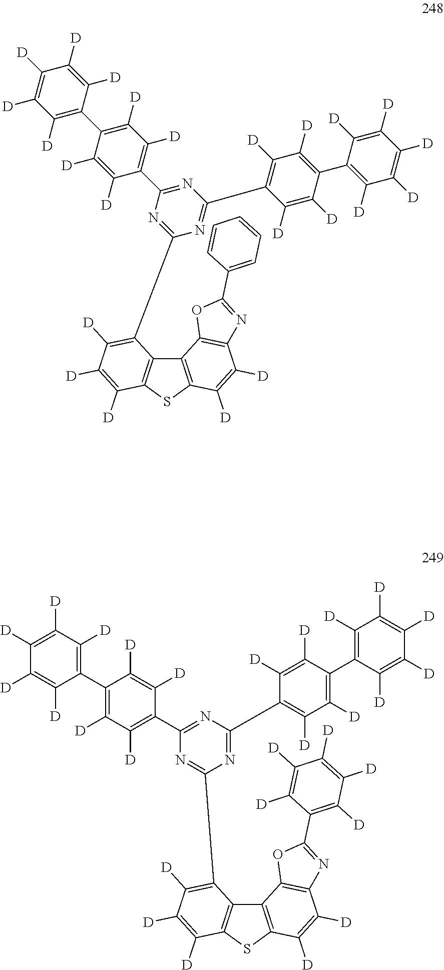 [SMILES: [2H]c1c([2H])c([2H])c(-c2c([2H])c([2H])c(-c3nc(-c4c([2H])c([2H])c(-c5c([2H])c([2H])c([2H])c([2H])c5[2H])c([2H])c4[2H])nc(-c4c([2H])c([2H])c([2H])c5sc6c([2H])c([2H])c7nc(-c8ccccc8)oc7c6c45)n3)c([2H])c2[2H])c([2H])c1[2H] ; [2H]c1c([2H])c([2H])c(-c2nc3c([2H])c([2H])c4sc5c([2H])c([2H])c([2H])c(-c6nc(-c7c([2H])c([2H])c(-c8c([2H])c([2H])c([2H])c([2H])c8[2H])c([2H])c7[2H])nc(-c7c([2H])c([2H])c(-c8c([2H])c([2H])c([2H])c([2H])c8[2H])c([2H])c7[2H])n6)c5c4c3o2)c([2H])c1[2H]]

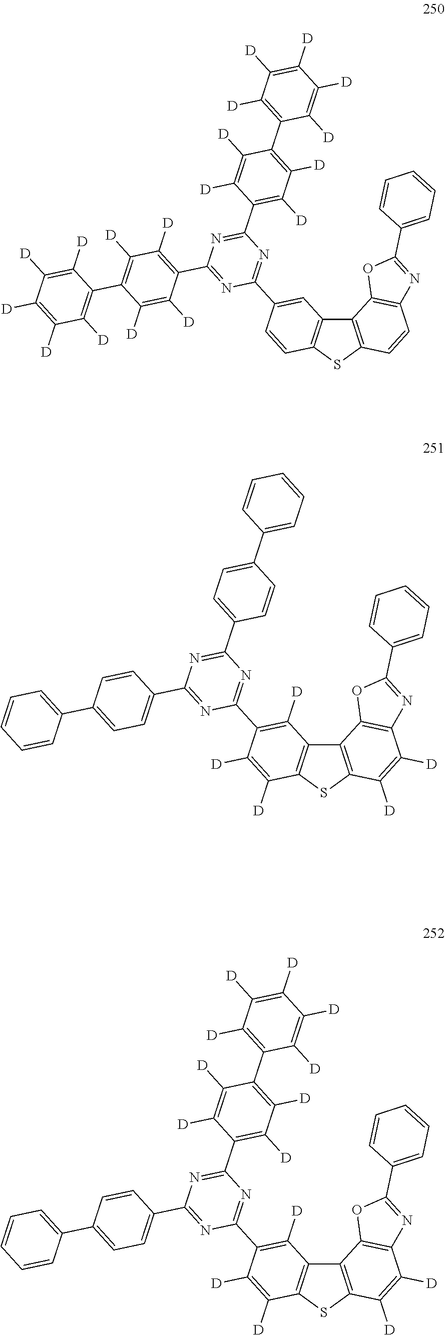 [SMILES: [2H]c1c([2H])c([2H])c(-c2c([2H])c([2H])c(-c3nc(-c4ccc5sc6ccc7nc(-c8ccccc8)oc7c6c5c4)nc(-c4c([2H])c([2H])c(-c5c([2H])c([2H])c([2H])c([2H])c5[2H])c([2H])c4[2H])n3)c([2H])c2[2H])c([2H])c1[2H] ; [2H]c1c(-c2nc(-c3ccc(-c4ccccc4)cc3)nc(-c3ccc(-c4ccccc4)cc3)n2)c([2H])c2c(sc3c([2H])c([2H])c4nc(-c5ccccc5)oc4c32)c1[2H] ; [2H]c1c([2H])c([2H])c(-c2c([2H])c([2H])c(-c3nc(-c4ccc(-c5ccccc5)cc4)nc(-c4c([2H])c([2H])c5sc6c([2H])c([2H])c7nc(-c8ccccc8)oc7c6c5c4[2H])n3)c([2H])c2[2H])c([2H])c1[2H]]

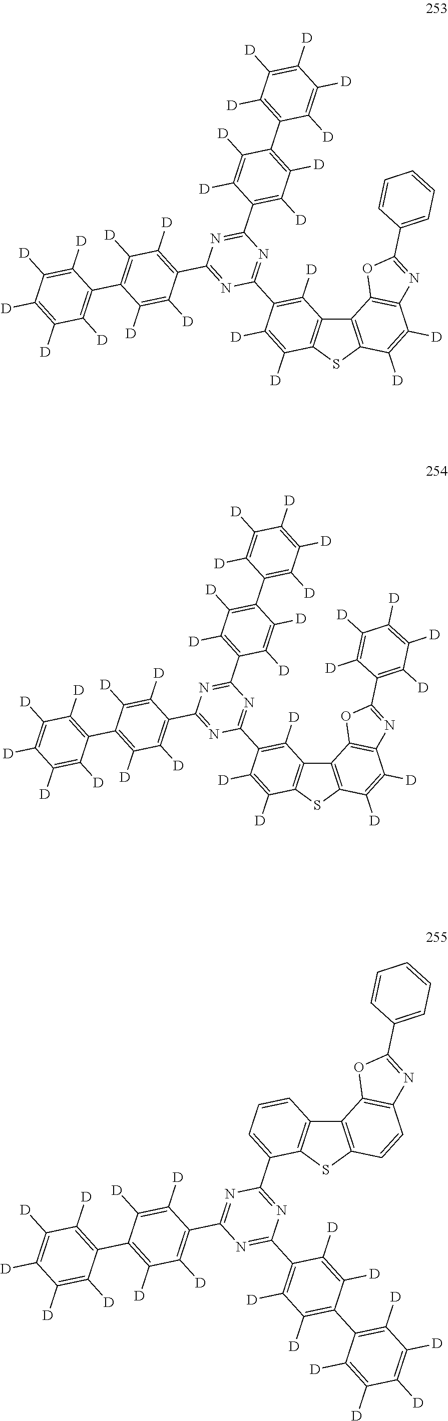

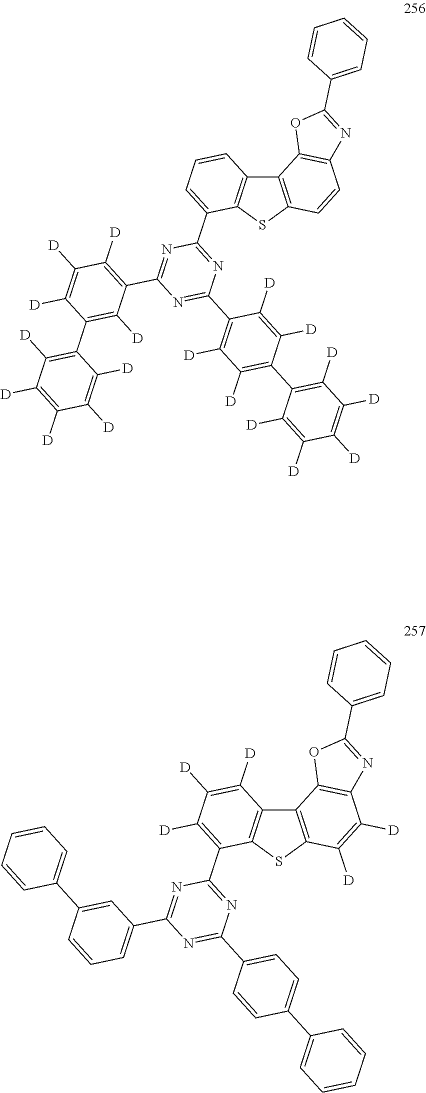 [SMILES: [2H]c1c([2H])c([2H])c(-c2c([2H])c([2H])c(-c3nc(-c4c([2H])c([2H])c([2H])c(-c5c([2H])c([2H])c([2H])c([2H])c5[2H])c4[2H])nc(-c4cccc5c4sc4ccc6nc(-c7ccccc7)oc6c45)n3)c([2H])c2[2H])c([2H])c1[2H] ; [2H]c1c([2H])c([2H])c2c(sc3c([2H])c([2H])c4nc(-c5ccccc5)oc4c32)c1-c1nc(-c2ccc(-c3ccccc3)cc2)nc(-c2cccc(-c3ccccc3)c2)n1]

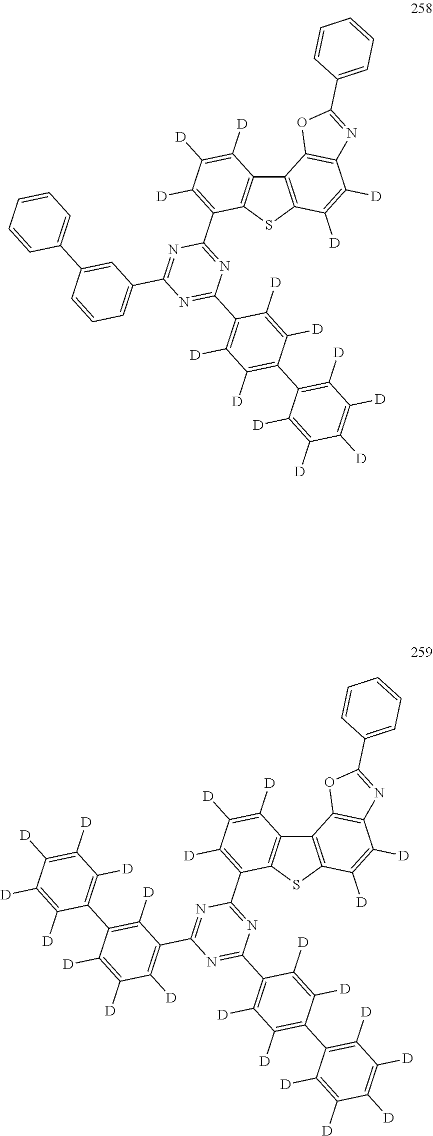 [SMILES: [2H]c1c([2H])c([2H])c(-c2c([2H])c([2H])c(-c3nc(-c4cccc(-c5ccccc5)c4)nc(-c4c([2H])c([2H])c([2H])c5c4sc4c([2H])c([2H])c6nc(-c7ccccc7)oc6c45)n3)c([2H])c2[2H])c([2H])c1[2H] ; [2H]c1c([2H])c([2H])c(-c2c([2H])c([2H])c(-c3nc(-c4c([2H])c([2H])c([2H])c(-c5c([2H])c([2H])c([2H])c([2H])c5[2H])c4[2H])nc(-c4c([2H])c([2H])c([2H])c5c4sc4c([2H])c([2H])c6nc(-c7ccccc7)oc6c45)n3)c([2H])c2[2H])c([2H])c1[2H]]

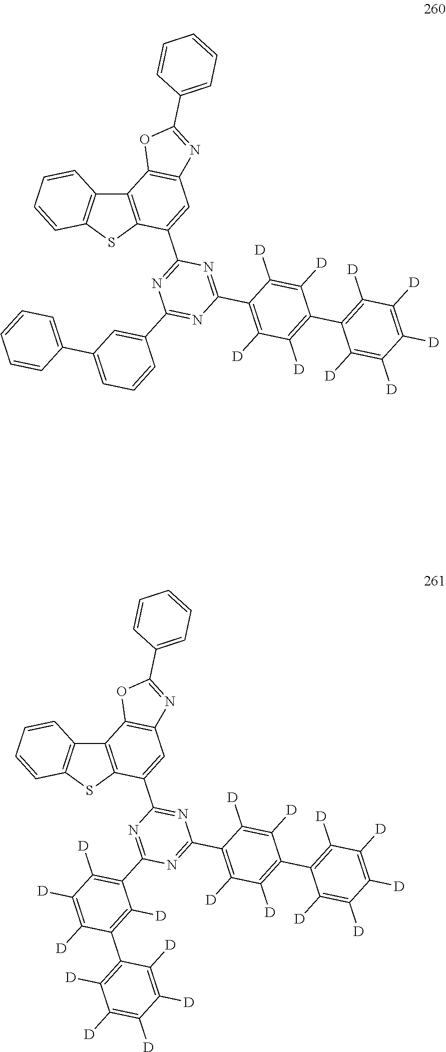 [SMILES: [2H]c1c([2H])c([2H])c(-c2c([2H])c([2H])c(-c3nc(-c4cccc(-c5ccccc5)c4)nc(-c4cc5nc(-c6ccccc6)oc5c5c4sc4ccccc45)n3)c([2H])c2[2H])c([2H])c1[2H] ; [2H]c1c([2H])c([2H])c(-c2c([2H])c([2H])c(-c3nc(-c4c([2H])c([2H])c([2H])c(-c5c([2H])c([2H])c([2H])c([2H])c5[2H])c4[2H])nc(-c4cc5nc(-c6ccccc6)oc5c5c4sc4ccccc45)n3)c([2H])c2[2H])c([2H])c1[2H]]

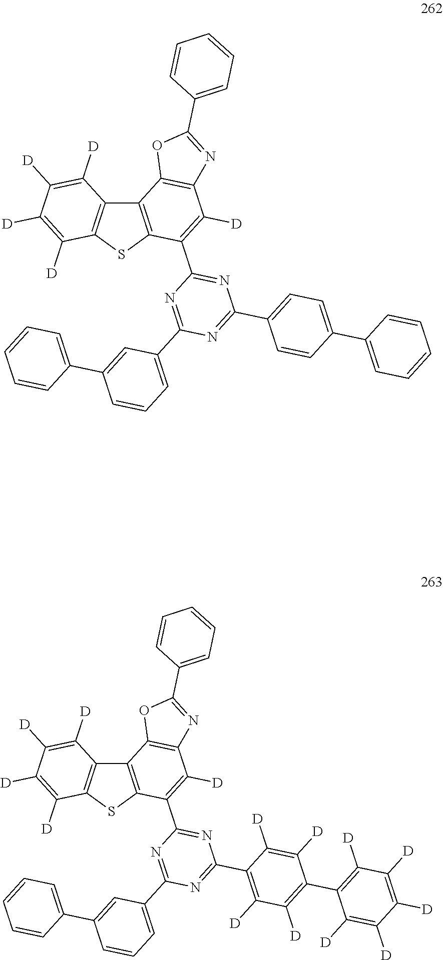 [SMILES: [2H]c1c([2H])c([2H])c2c(sc3c(-c4nc(-c5ccc(-c6ccccc6)cc5)nc(-c5cccc(-c6ccccc6)c5)n4)c([2H])c4nc(-c5ccccc5)oc4c32)c1[2H] ; [2H]c1c([2H])c([2H])c(-c2c([2H])c([2H])c(-c3nc(-c4cccc(-c5ccccc5)c4)nc(-c4c([2H])c5nc(-c6ccccc6)oc5c5c4sc4c([2H])c([2H])c([2H])c([2H])c45)n3)c([2H])c2[2H])c([2H])c1[2H]]

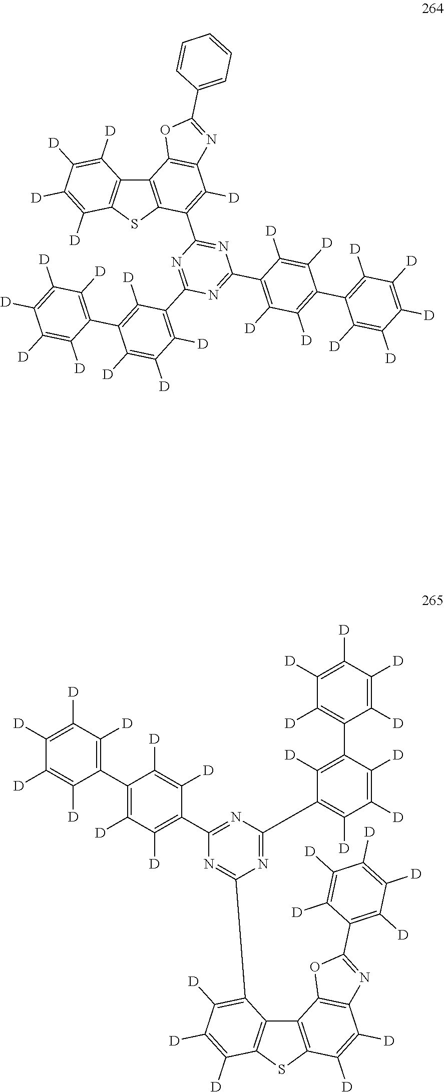 [SMILES: [2H]c1c([2H])c([2H])c(-c2c([2H])c([2H])c(-c3nc(-c4c([2H])c([2H])c([2H])c(-c5c([2H])c([2H])c([2H])c([2H])c5[2H])c4[2H])nc(-c4c([2H])c5nc(-c6ccccc6)oc5c5c4sc4c([2H])c([2H])c([2H])c([2H])c45)n3)c([2H])c2[2H])c([2H])c1[2H] ; [2H]c1c([2H])c([2H])c(-c2nc3c([2H])c([2H])c4sc5c([2H])c([2H])c([2H])c(-c6nc(-c7c([2H])c([2H])c(-c8c([2H])c([2H])c([2H])c([2H])c8[2H])c([2H])c7[2H])nc(-c7c([2H])c([2H])c([2H])c(-c8c([2H])c([2H])c([2H])c([2H])c8[2H])c7[2H])n6)c5c4c3o2)c([2H])c1[2H]]

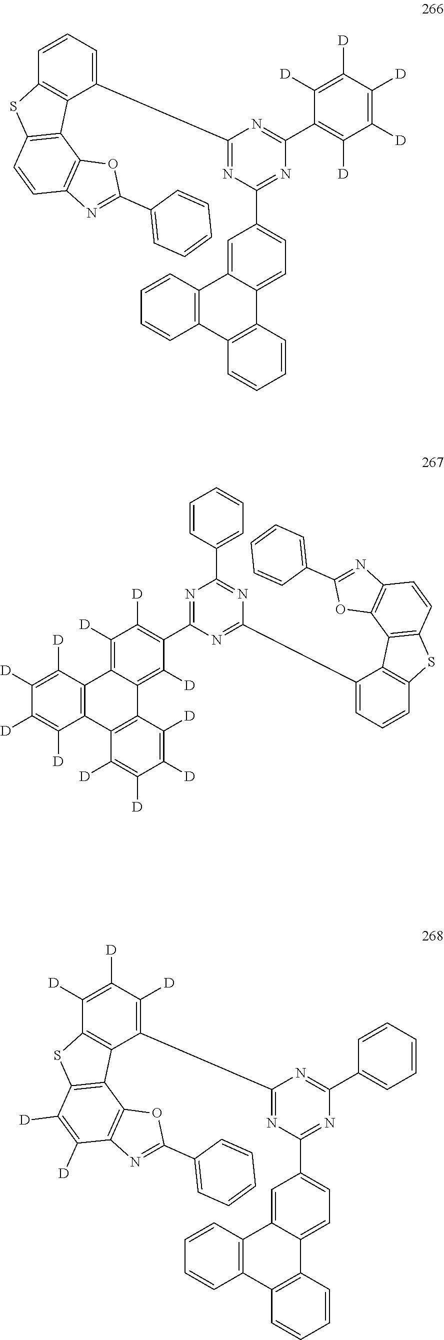 [SMILES: [2H]c1c([2H])c([2H])c(-c2nc(-c3ccc4c5ccccc5c5ccccc5c4c3)nc(-c3cccc4sc5ccc6nc(-c7ccccc7)oc6c5c34)n2)c([2H])c1[2H] ; [2H]c1c([2H])c([2H])c2c(c1[2H])c1c([2H])c([2H])c([2H])c([2H])c1c1c([2H])c(-c3nc(-c4ccccc4)nc(-c4cccc5sc6ccc7nc(-c8ccccc8)oc7c6c45)n3)c([2H])c([2H])c21 ; [2H]c1c([2H])c(-c2nc(-c3ccccc3)nc(-c3ccc4c5ccccc5c5ccccc5c4c3)n2)c2c(sc3c([2H])c([2H])c4nc(-c5ccccc5)oc4c32)c1[2H]]

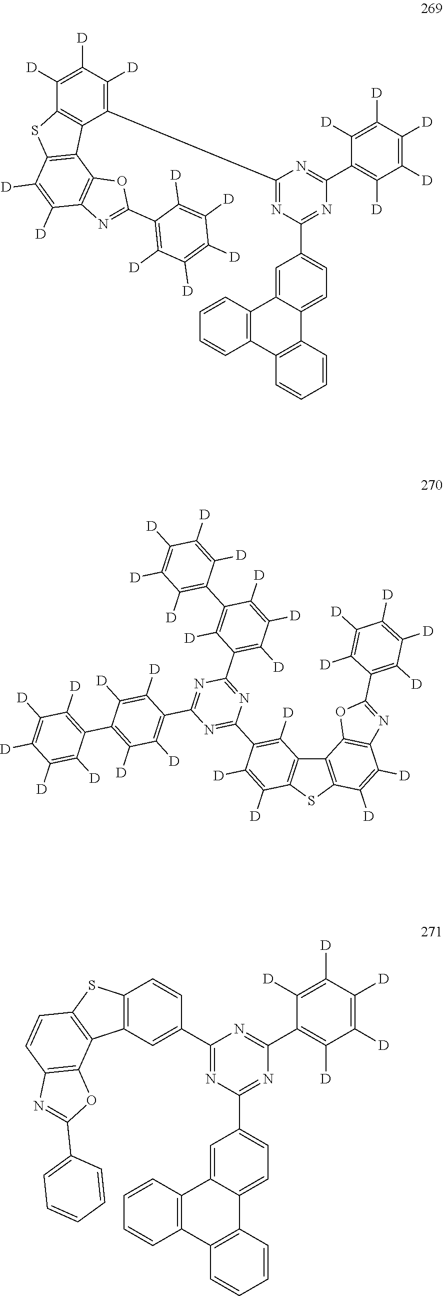

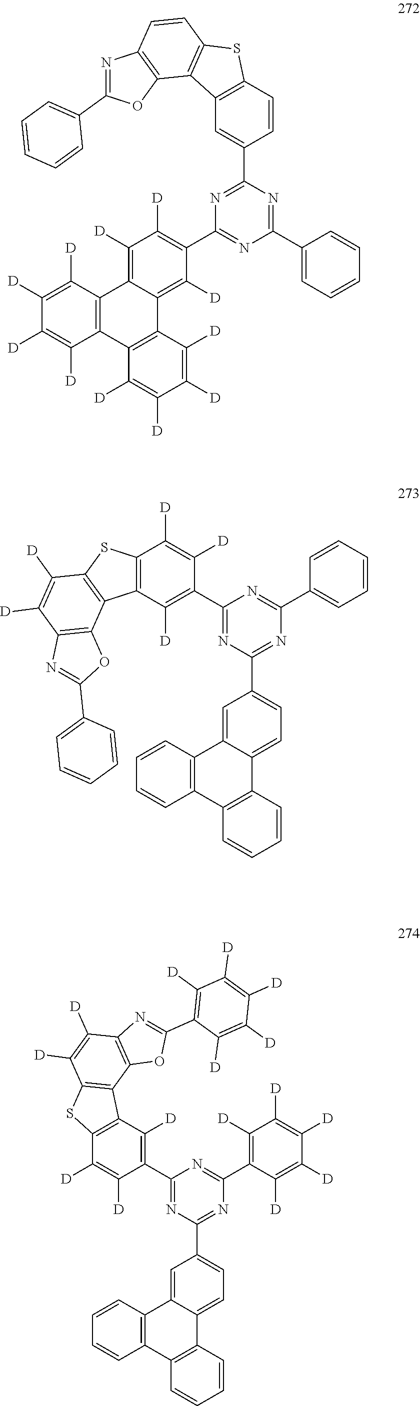 [SMILES: [2H]c1c([2H])c([2H])c2c(c1[2H])c1c([2H])c([2H])c([2H])c([2H])c1c1c([2H])c(-c3nc(-c4ccccc4)nc(-c4ccc5sc6ccc7nc(-c8ccccc8)oc7c6c5c4)n3)c([2H])c([2H])c21 ; [2H]c1c(-c2nc(-c3ccccc3)nc(-c3ccc4c5ccccc5c5ccccc5c4c3)n2)c([2H])c2c(sc3c([2H])c([2H])c4nc(-c5ccccc5)oc4c32)c1[2H] ; [2H]c1c([2H])c([2H])c(-c2nc(-c3ccc4c5ccccc5c5ccccc5c4c3)nc(-c3c([2H])c([2H])c4sc5c([2H])c([2H])c6nc(-c7c([2H])c([2H])c([2H])c([2H])c7[2H])oc6c5c4c3[2H])n2)c([2H])c1[2H]]

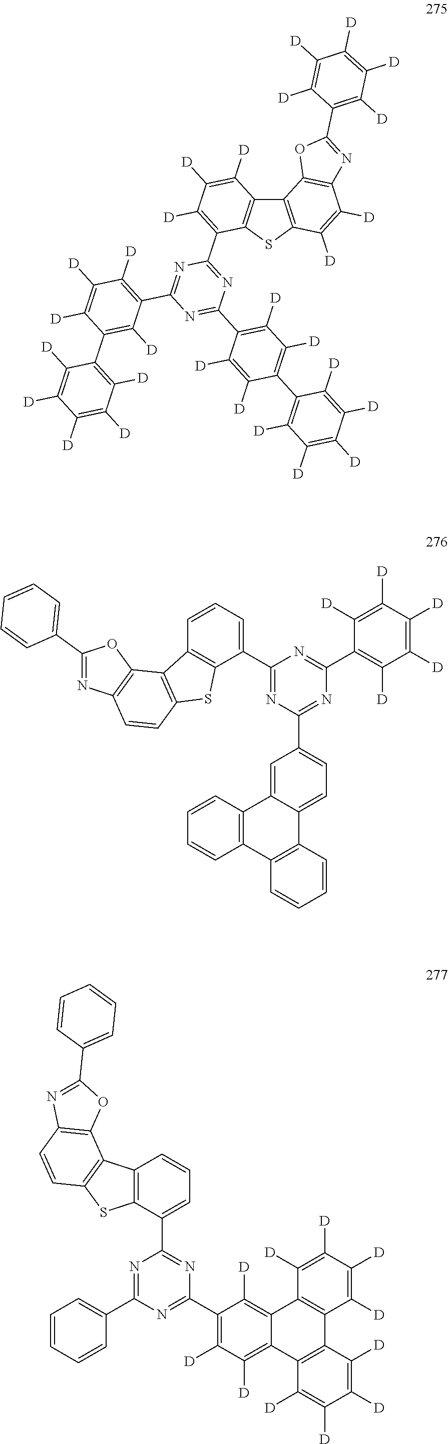

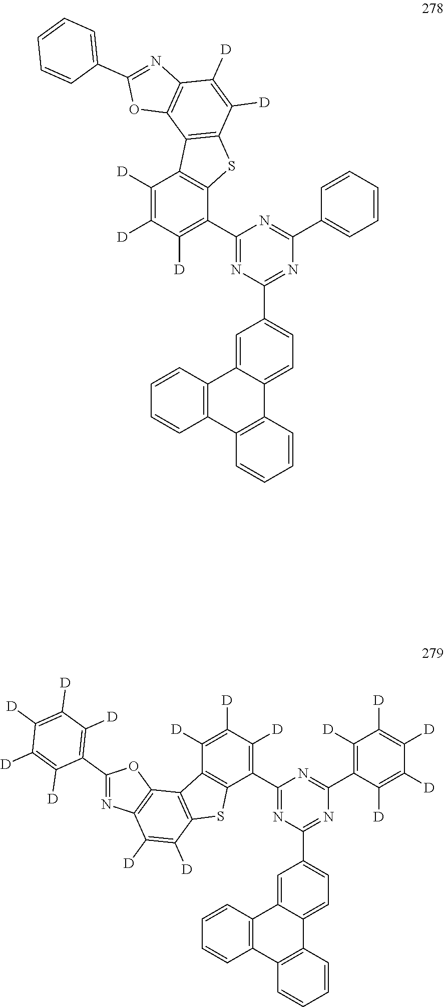 [SMILES: [2H]c1c([2H])c([2H])c2c(sc3c([2H])c([2H])c4nc(-c5ccccc5)oc4c32)c1-c1nc(-c2ccccc2)nc(-c2ccc3c4ccccc4c4ccccc4c3c2)n1 ; [2H]c1c([2H])c([2H])c(-c2nc(-c3ccc4c5ccccc5c5ccccc5c4c3)nc(-c3c([2H])c([2H])c([2H])c4c3sc3c([2H])c([2H])c5nc(-c6c([2H])c([2H])c([2H])c([2H])c6[2H])oc5c34)n2)c([2H])c1[2H]]

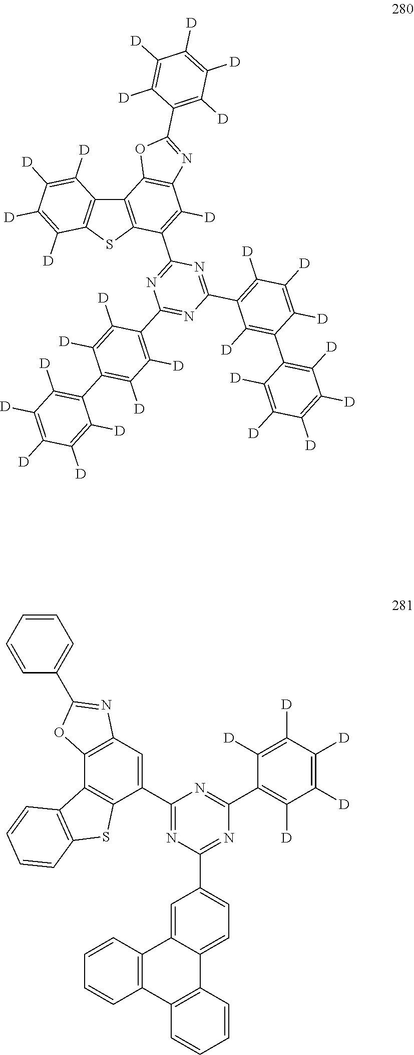 [SMILES: [2H]c1c([2H])c([2H])c(-c2nc3c([2H])c(-c4nc(-c5c([2H])c([2H])c(-c6c([2H])c([2H])c([2H])c([2H])c6[2H])c([2H])c5[2H])nc(-c5c([2H])c([2H])c([2H])c(-c6c([2H])c([2H])c([2H])c([2H])c6[2H])c5[2H])n4)c4sc5c([2H])c([2H])c([2H])c([2H])c5c4c3o2)c([2H])c1[2H] ; [2H]c1c([2H])c([2H])c(-c2nc(-c3ccc4c5ccccc5c5ccccc5c4c3)nc(-c3cc4nc(-c5ccccc5)oc4c4c3sc3ccccc34)n2)c([2H])c1[2H]]

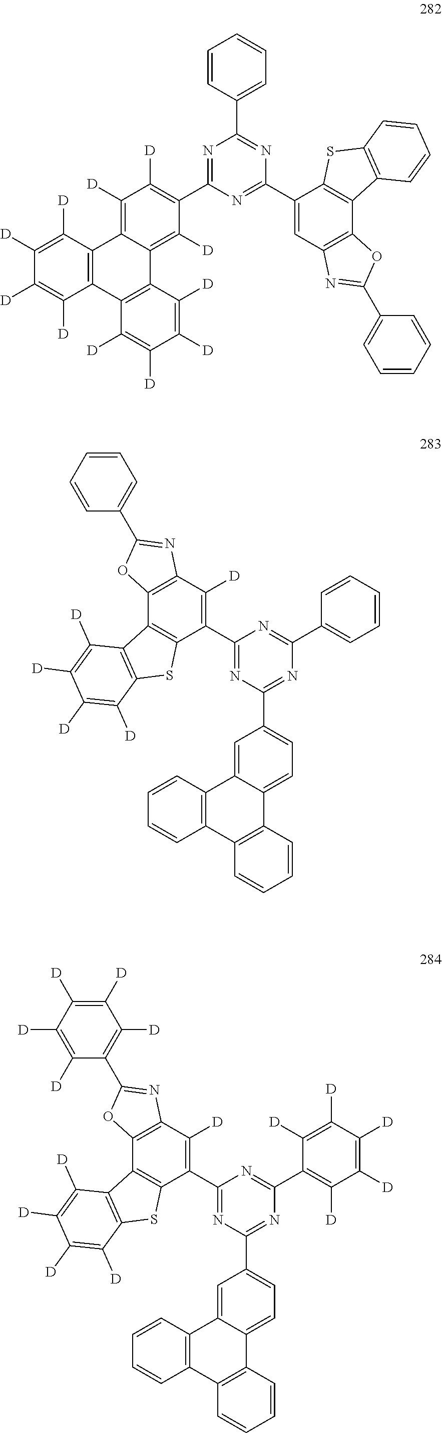 [SMILES: [2H]c1c([2H])c([2H])c2c(c1[2H])c1c([2H])c([2H])c([2H])c([2H])c1c1c([2H])c(-c3nc(-c4ccccc4)nc(-c4cc5nc(-c6ccccc6)oc5c5c4sc4ccccc45)n3)c([2H])c([2H])c21 ; [2H]c1c([2H])c([2H])c2c(sc3c(-c4nc(-c5ccccc5)nc(-c5ccc6c7ccccc7c7ccccc7c6c5)n4)c([2H])c4nc(-c5ccccc5)oc4c32)c1[2H] ; [2H]c1c([2H])c([2H])c(-c2nc(-c3ccc4c5ccccc5c5ccccc5c4c3)nc(-c3c([2H])c4nc(-c5c([2H])c([2H])c([2H])c([2H])c5[2H])oc4c4c3sc3c([2H])c([2H])c([2H])c([2H])c34)n2)c([2H])c1[2H]]

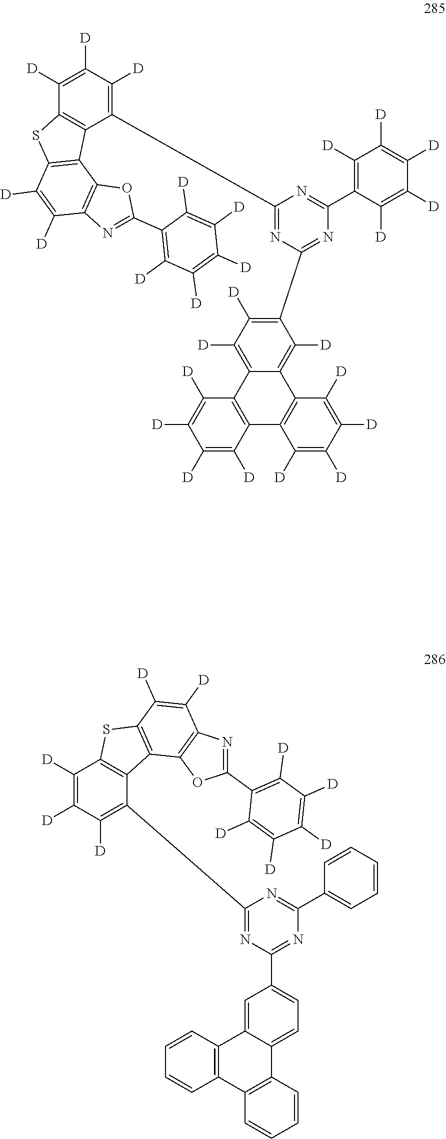 [SMILES: [2H]c1c([2H])c([2H])c(-c2nc(-c3c([2H])c([2H])c4c5c([2H])c([2H])c([2H])c([2H])c5c5c([2H])c([2H])c([2H])c([2H])c5c4c3[2H])nc(-c3c([2H])c([2H])c([2H])c4sc5c([2H])c([2H])c6nc(-c7c([2H])c([2H])c([2H])c([2H])c7[2H])oc6c5c34)n2)c([2H])c1[2H] ; [2H]c1c([2H])c([2H])c(-c2nc3c([2H])c([2H])c4sc5c([2H])c([2H])c([2H])c(-c6nc(-c7ccccc7)nc(-c7ccc8c9ccccc9c9ccccc9c8c7)n6)c5c4c3o2)c([2H])c1[2H]]

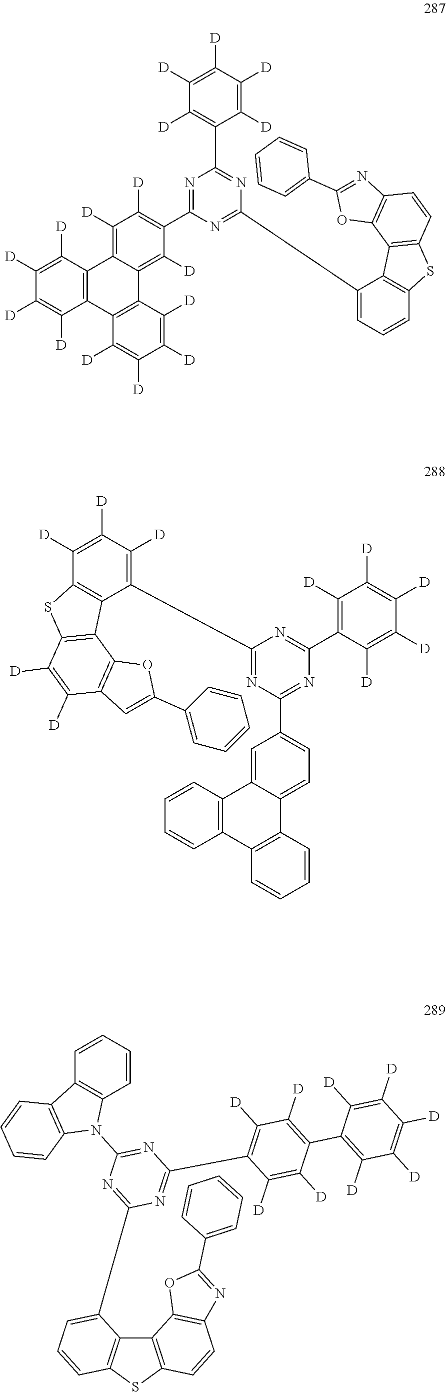 [SMILES: [2H]c1c([2H])c([2H])c(-c2nc(-c3c([2H])c([2H])c4c5c([2H])c([2H])c([2H])c([2H])c5c5c([2H])c([2H])c([2H])c([2H])c5c4c3[2H])nc(-c3cccc4sc5ccc6nc(-c7ccccc7)oc6c5c34)n2)c([2H])c1[2H] ; [2H]c1c([2H])c([2H])c(-c2nc(-c3ccc4c5ccccc5c5ccccc5c4c3)nc(-c3c([2H])c([2H])c([2H])c4sc5c([2H])c([2H])c6cc(-c7ccccc7)oc6c5c34)n2)c([2H])c1[2H] ; [2H]c1c([2H])c([2H])c(-c2c([2H])c([2H])c(-c3nc(-c4cccc5sc6ccc7nc(-c8ccccc8)oc7c6c45)nc(-n4c5ccccc5c5ccccc54)n3)c([2H])c2[2H])c([2H])c1[2H]]

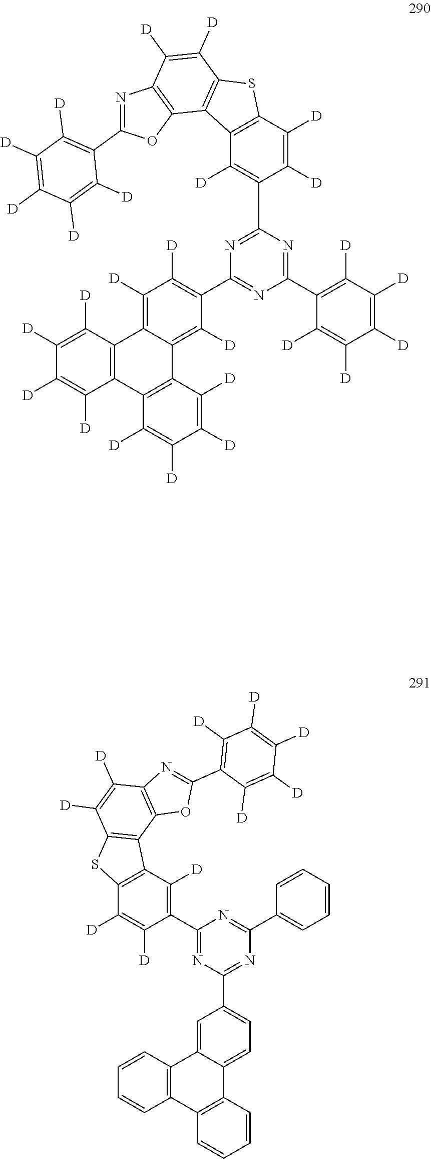 [SMILES: [2H]c1c([2H])c([2H])c(-c2nc(-c3c([2H])c([2H])c4c5c([2H])c([2H])c([2H])c([2H])c5c5c([2H])c([2H])c([2H])c([2H])c5c4c3[2H])nc(-c3c([2H])c([2H])c4sc5c([2H])c([2H])c6nc(-c7c([2H])c([2H])c([2H])c([2H])c7[2H])oc6c5c4c3[2H])n2)c([2H])c1[2H] ; [2H]c1c([2H])c([2H])c(-c2nc3c([2H])c([2H])c4sc5c([2H])c([2H])c(-c6nc(-c7ccccc7)nc(-c7ccc8c9ccccc9c9ccccc9c8c7)n6)c([2H])c5c4c3o2)c([2H])c1[2H]]

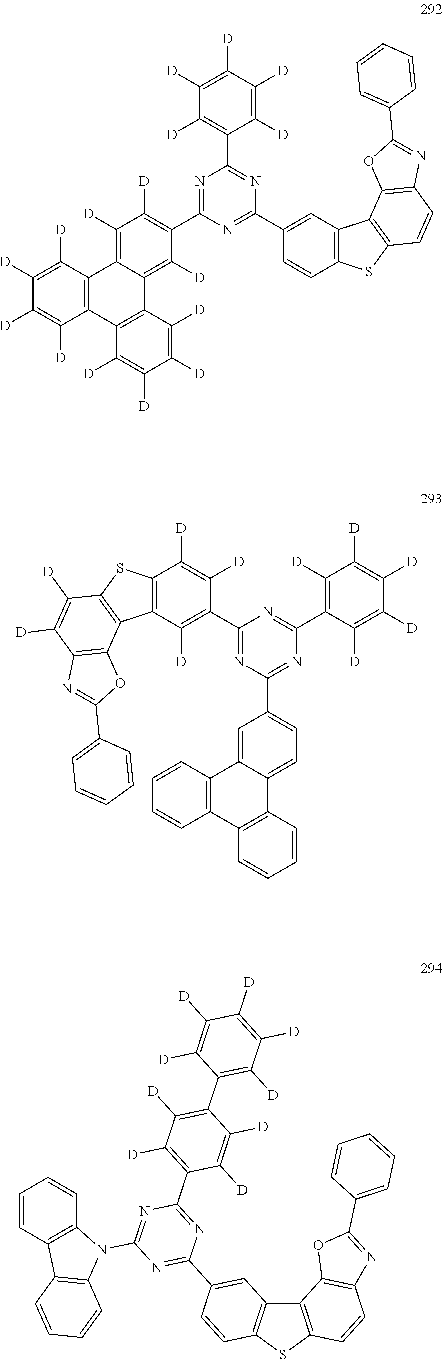 [SMILES: [2H]c1c([2H])c([2H])c(-c2nc(-c3ccc4sc5ccc6nc(-c7ccccc7)oc6c5c4c3)nc(-c3c([2H])c([2H])c4c5c([2H])c([2H])c([2H])c([2H])c5c5c([2H])c([2H])c([2H])c([2H])c5c4c3[2H])n2)c([2H])c1[2H] ; [2H]c1c([2H])c([2H])c(-c2nc(-c3ccc4c5ccccc5c5ccccc5c4c3)nc(-c3c([2H])c([2H])c4sc5c([2H])c([2H])c6nc(-c7ccccc7)oc6c5c4c3[2H])n2)c([2H])c1[2H] ; [2H]c1c([2H])c([2H])c(-c2c([2H])c([2H])c(-c3nc(-c4ccc5sc6ccc7nc(-c8ccccc8)oc7c6c5c4)nc(-n4c5ccccc5c5ccccc54)n3)c([2H])c2[2H])c([2H])c1[2H]]

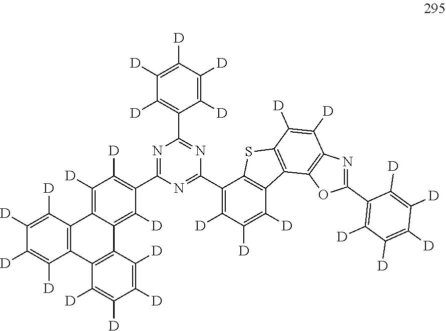 [SMILES: [2H]c1c([2H])c([2H])c(-c2nc(-c3c([2H])c([2H])c4c5c([2H])c([2H])c([2H])c([2H])c5c5c([2H])c([2H])c([2H])c([2H])c5c4c3[2H])nc(-c3c([2H])c([2H])c([2H])c4c3sc3c([2H])c([2H])c5nc(-c6c([2H])c([2H])c([2H])c([2H])c6[2H])oc5c34)n2)c([2H])c1[2H]]

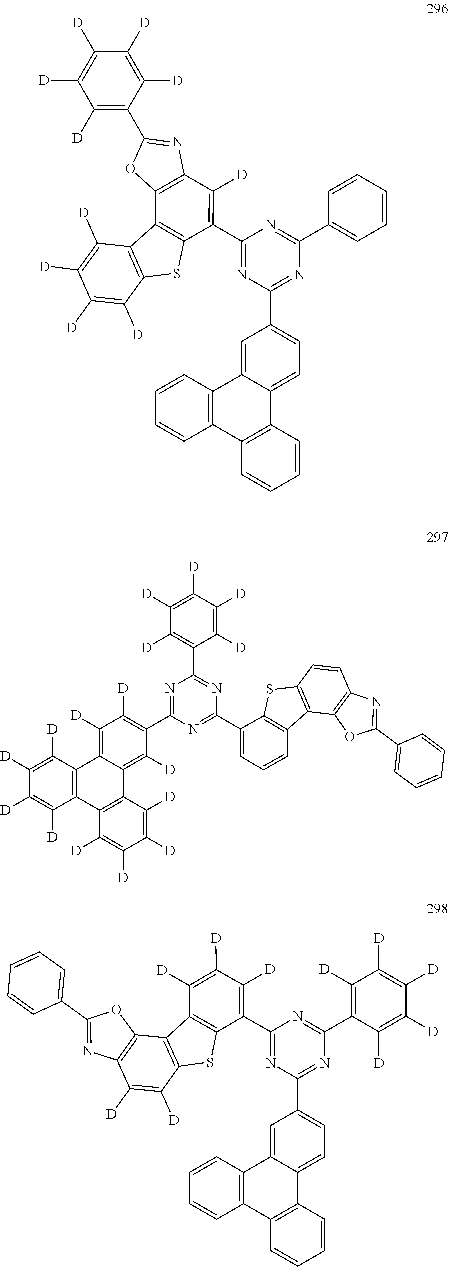 [SMILES: [2H]c1c([2H])c([2H])c(-c2nc3c([2H])c(-c4nc(-c5ccccc5)nc(-c5ccc6c7ccccc7c7ccccc7c6c5)n4)c4sc5c([2H])c([2H])c([2H])c([2H])c5c4c3o2)c([2H])c1[2H] ; [2H]c1c([2H])c([2H])c(-c2nc(-c3c([2H])c([2H])c4c5c([2H])c([2H])c([2H])c([2H])c5c5c([2H])c([2H])c([2H])c([2H])c5c4c3[2H])nc(-c3cccc4c3sc3ccc5nc(-c6ccccc6)oc5c34)n2)c([2H])c1[2H] ; [2H]c1c([2H])c([2H])c(-c2nc(-c3ccc4c5ccccc5c5ccccc5c4c3)nc(-c3c([2H])c([2H])c([2H])c4c3sc3c([2H])c([2H])c5nc(-c6ccccc6)oc5c34)n2)c([2H])c1[2H]]

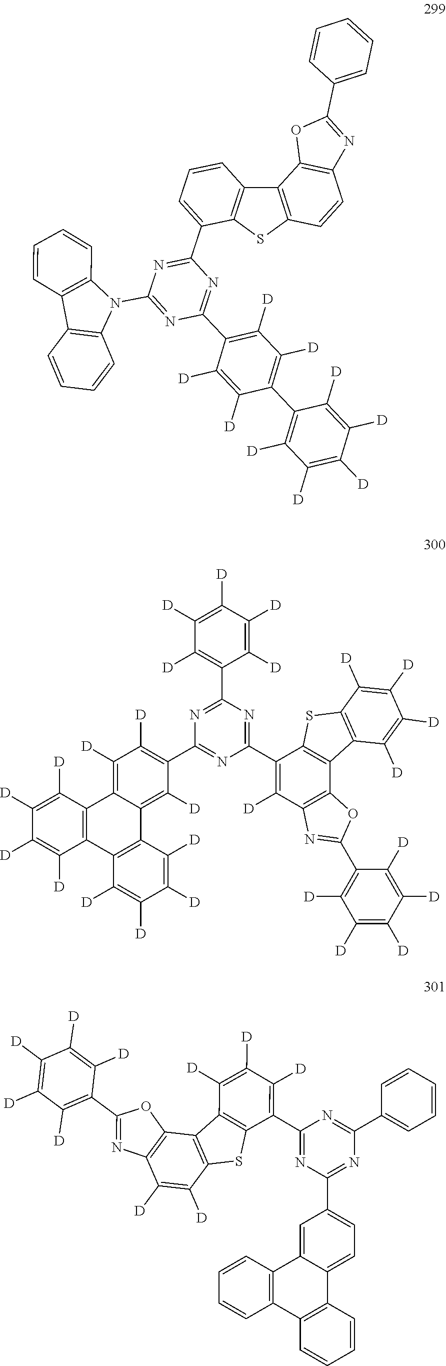

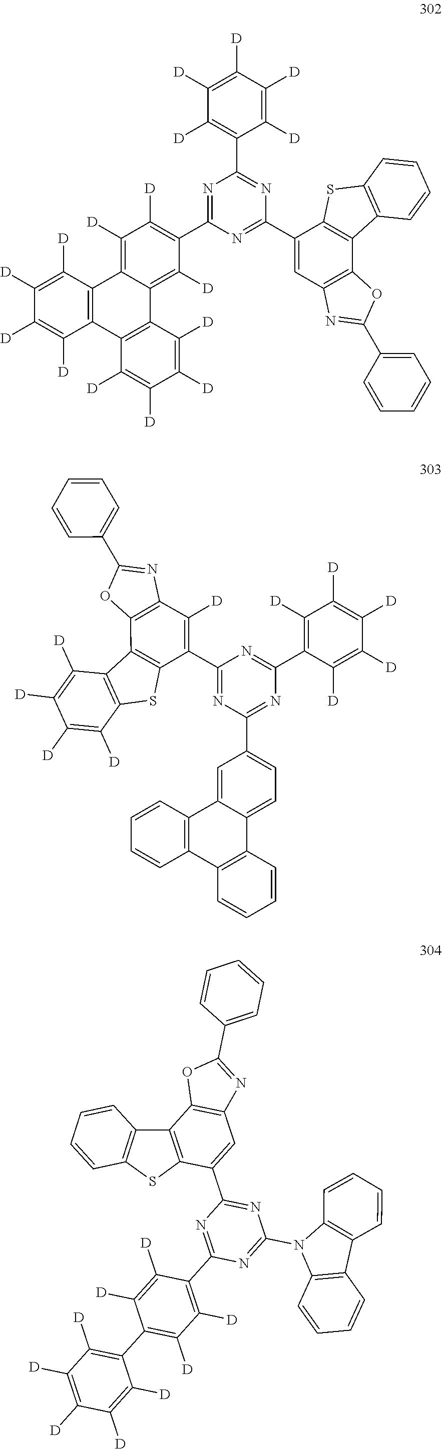

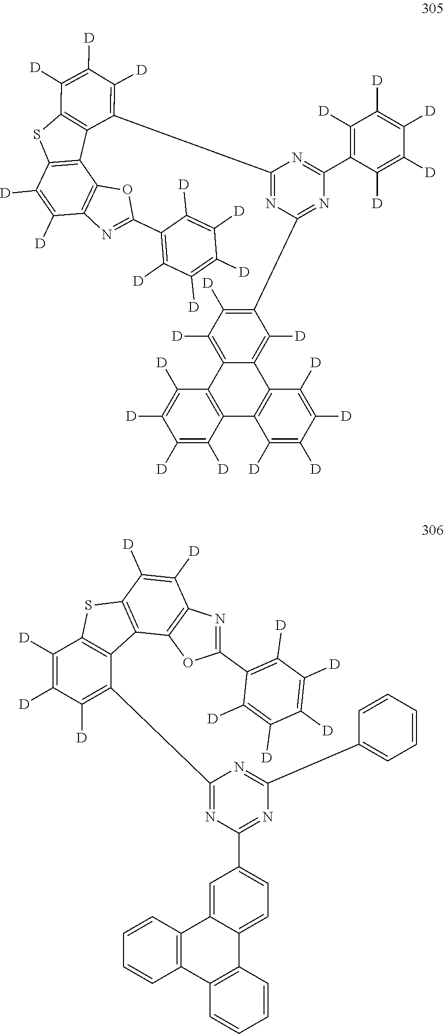 [SMILES: [2H]c1c([2H])c([2H])c(-c2nc(-c3c([2H])c([2H])c4c5c([2H])c([2H])c([2H])c([2H])c5c5c([2H])c([2H])c([2H])c([2H])c5c4c3[2H])nc(-c3c([2H])c([2H])c([2H])c4sc5c([2H])c([2H])c6nc(-c7c([2H])c([2H])c([2H])c([2H])c7[2H])oc6c5c34)n2)c([2H])c1[2H] ; [2H]c1c([2H])c([2H])c(-c2nc3c([2H])c([2H])c4sc5c([2H])c([2H])c([2H])c(-c6nc(-c7ccccc7)nc(-c7ccc8c9ccccc9c9ccccc9c8c7)n6)c5c4c3o2)c([2H])c1[2H]]

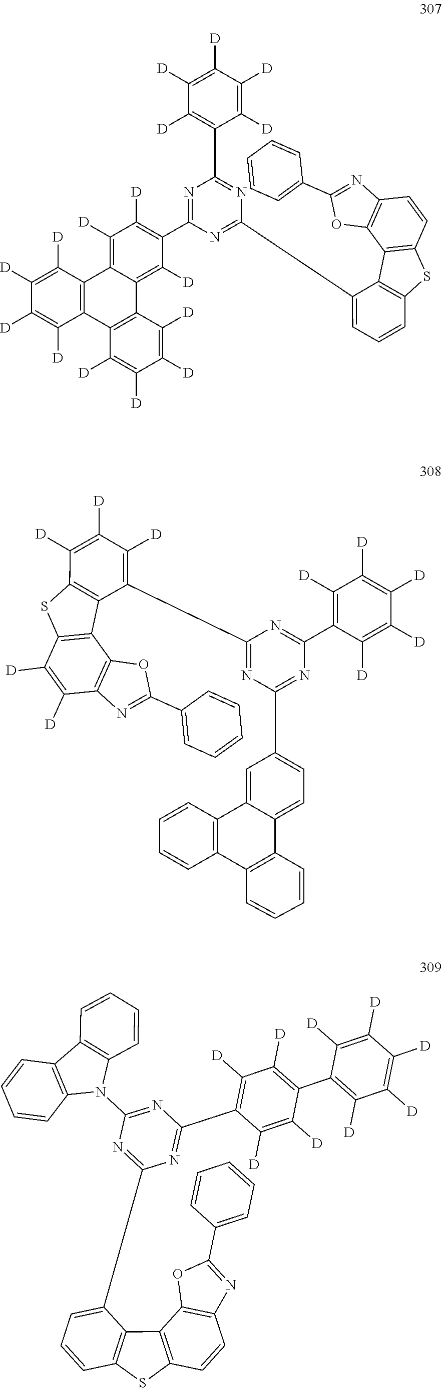 [SMILES: [2H]c1c([2H])c([2H])c(-c2nc(-c3c([2H])c([2H])c4c5c([2H])c([2H])c([2H])c([2H])c5c5c([2H])c([2H])c([2H])c([2H])c5c4c3[2H])nc(-c3cccc4sc5ccc6nc(-c7ccccc7)oc6c5c34)n2)c([2H])c1[2H] ; [2H]c1c([2H])c([2H])c(-c2nc(-c3ccc4c5ccccc5c5ccccc5c4c3)nc(-c3c([2H])c([2H])c([2H])c4sc5c([2H])c([2H])c6nc(-c7ccccc7)oc6c5c34)n2)c([2H])c1[2H] ; [2H]c1c([2H])c([2H])c(-c2c([2H])c([2H])c(-c3nc(-c4cccc5sc6ccc7nc(-c8ccccc8)oc7c6c45)nc(-n4c5ccccc5c5ccccc54)n3)c([2H])c2[2H])c([2H])c1[2H]]

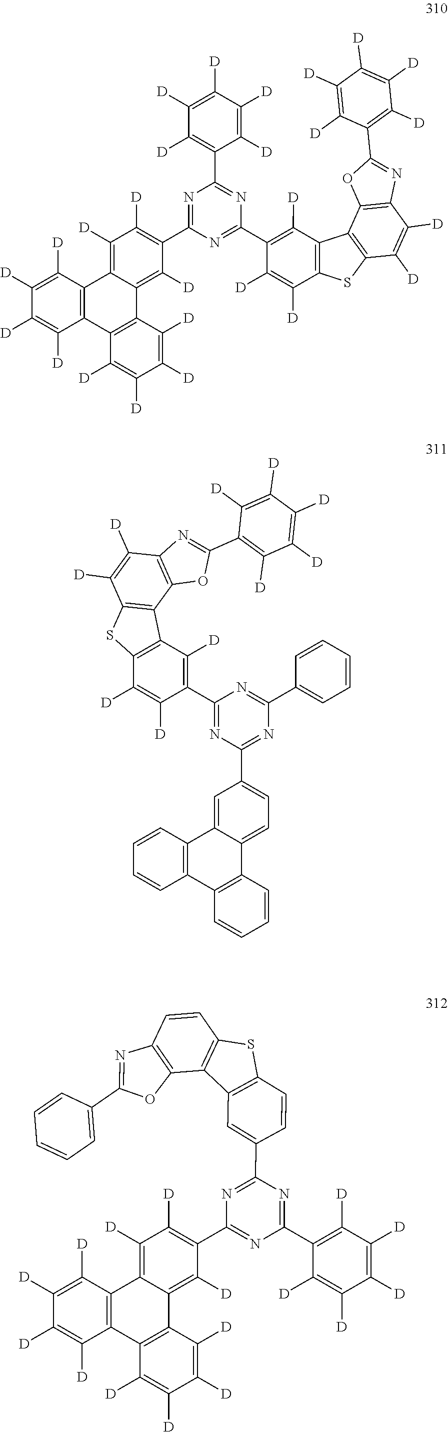

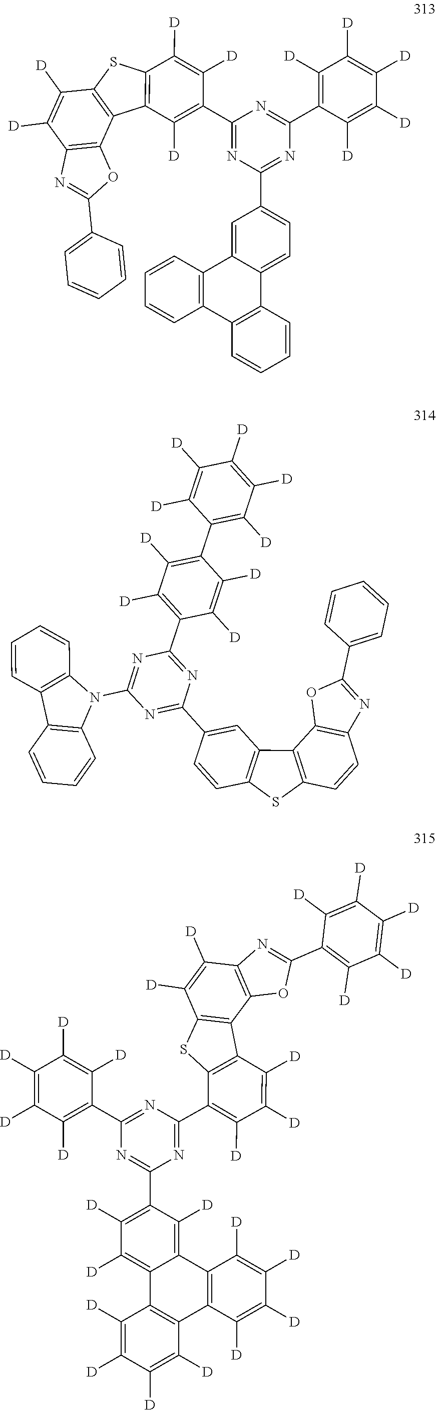

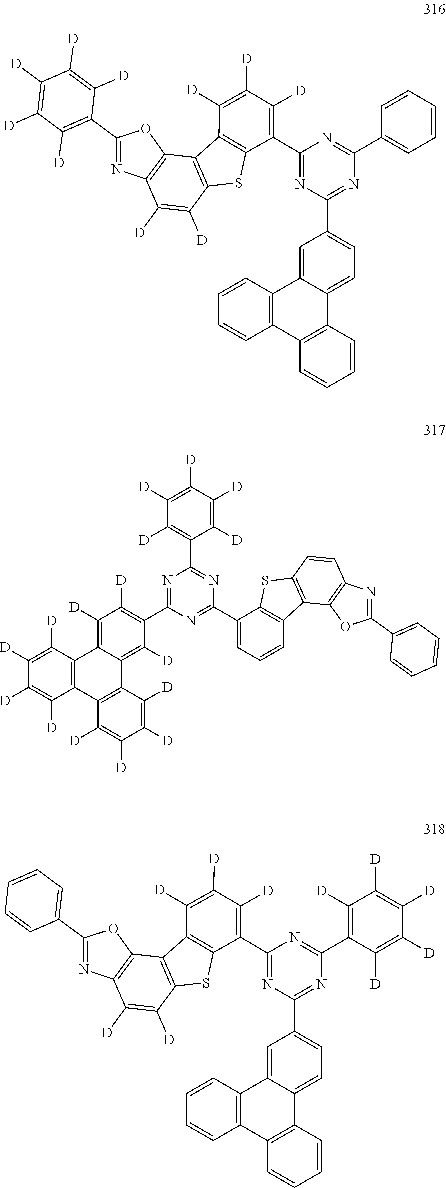 [SMILES: [2H]c1c([2H])c([2H])c(-c2nc3c([2H])c([2H])c4sc5c(-c6nc(-c7ccccc7)nc(-c7ccc8c9ccccc9c9ccccc9c8c7)n6)c([2H])c([2H])c([2H])c5c4c3o2)c([2H])c1[2H] ; [2H]c1c([2H])c([2H])c(-c2nc(-c3c([2H])c([2H])c4c5c([2H])c([2H])c([2H])c([2H])c5c5c([2H])c([2H])c([2H])c([2H])c5c4c3[2H])nc(-c3cccc4c3sc3ccc5nc(-c6ccccc6)oc5c34)n2)c([2H])c1[2H] ; [2H]c1c([2H])c([2H])c(-c2nc(-c3ccc4c5ccccc5c5ccccc5c4c3)nc(-c3c([2H])c([2H])c([2H])c4c3sc3c([2H])c([2H])c5nc(-c6ccccc6)oc5c34)n2)c([2H])c1[2H]]

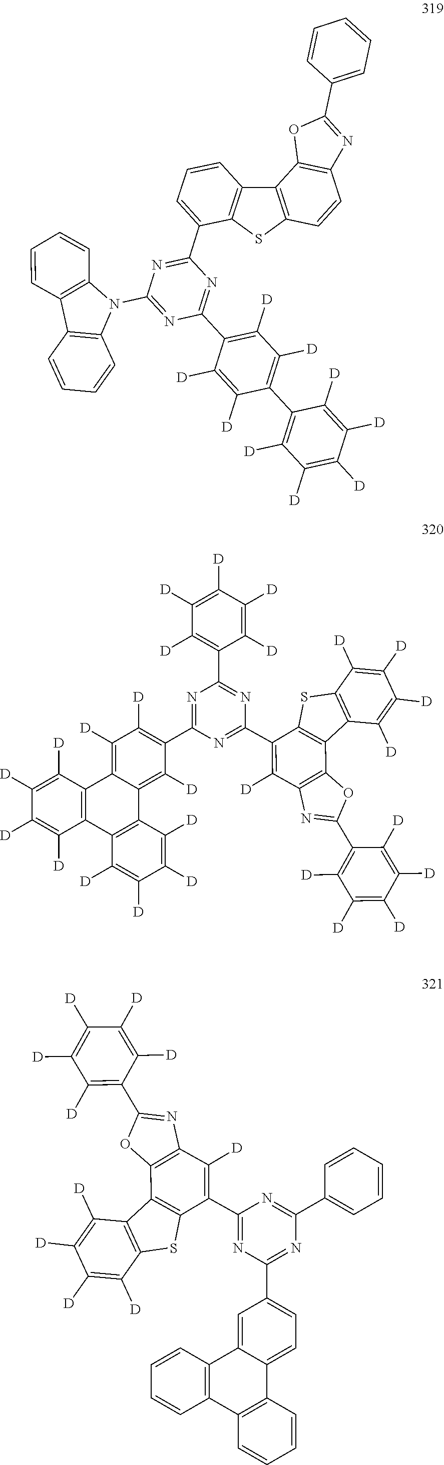

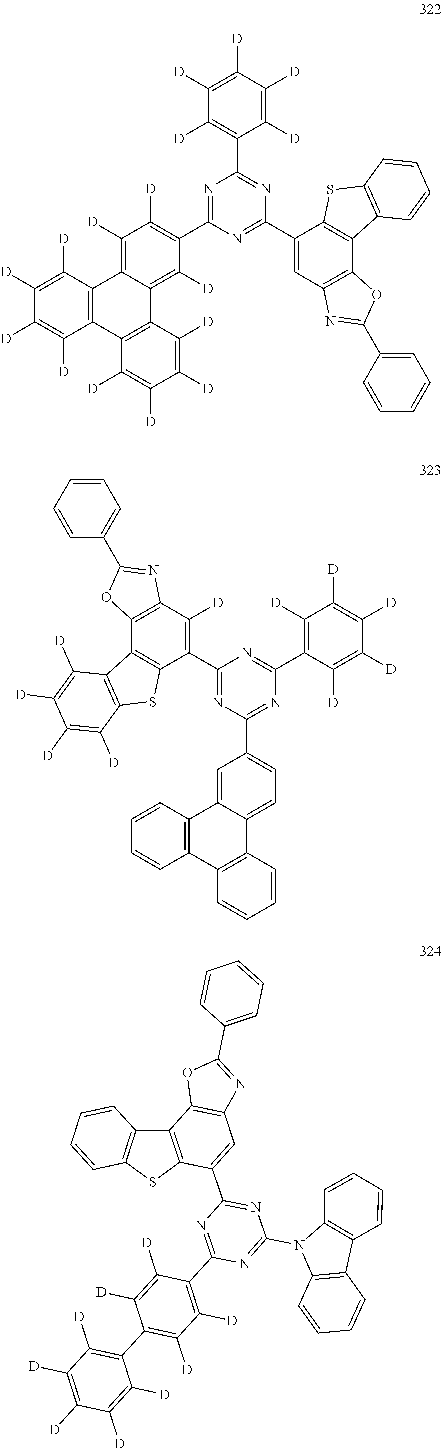

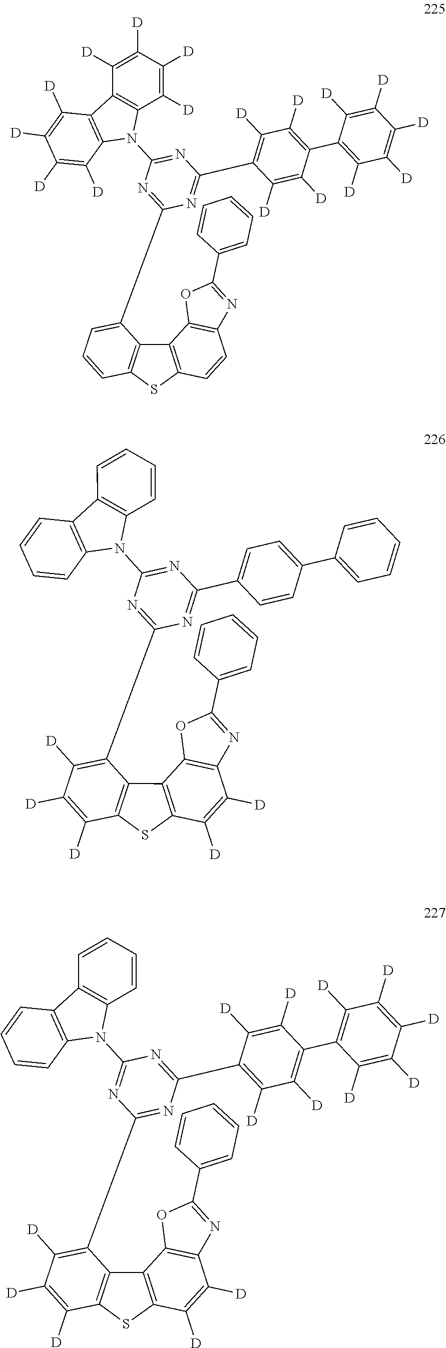 [SMILES: [2H]c1c([2H])c([2H])c(-c2c([2H])c([2H])c(-c3nc(-c4cccc5sc6ccc7nc(-c8ccccc8)oc7c6c45)nc(-n4c5c([2H])c([2H])c([2H])c([2H])c5c5c([2H])c([2H])c([2H])c([2H])c54)n3)c([2H])c2[2H])c([2H])c1[2H] ; [2H]c1c([2H])c(-c2nc(-c3ccc(-c4ccccc4)cc3)nc(-n3c4ccccc4c4ccccc43)n2)c2c(sc3c([2H])c([2H])c4nc(-c5ccccc5)oc4c32)c1[2H] ; [2H]c1c([2H])c([2H])c(-c2c([2H])c([2H])c(-c3nc(-c4c([2H])c([2H])c([2H])c5sc6c([2H])c([2H])c7nc(-c8ccccc8)oc7c6c45)nc(-n4c5ccccc5c5ccccc54)n3)c([2H])c2[2H])c([2H])c1[2H]]

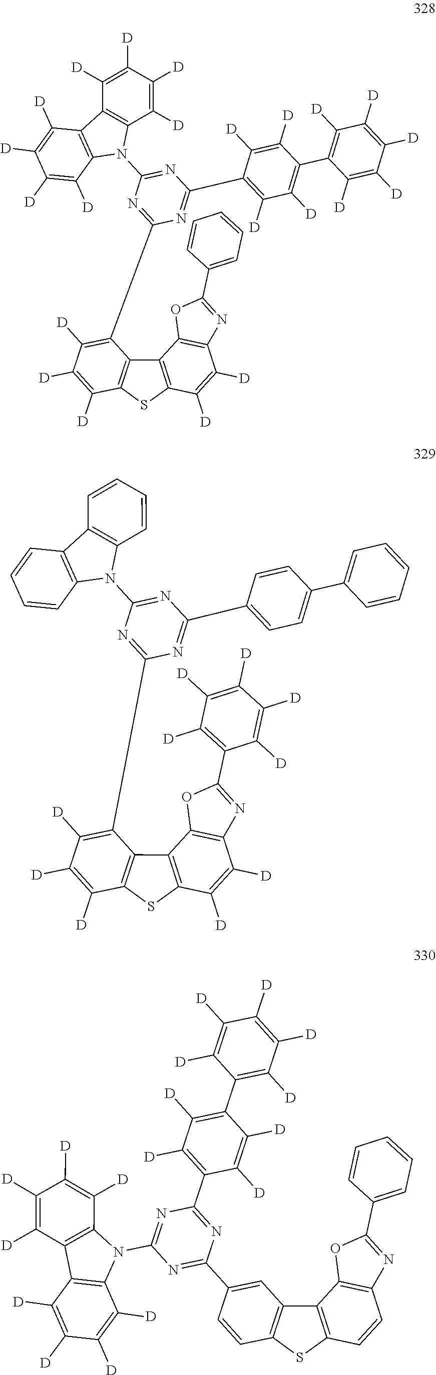

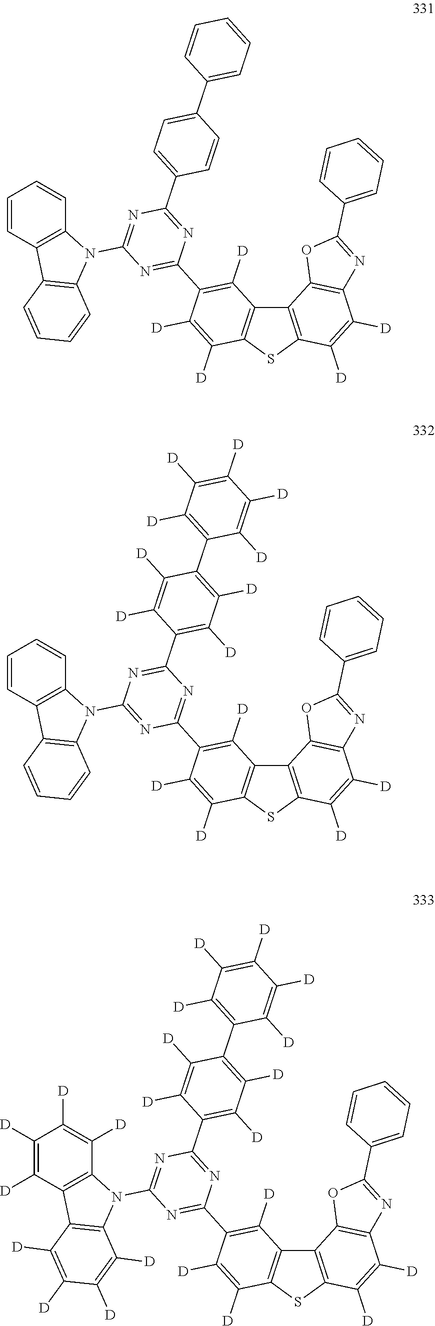

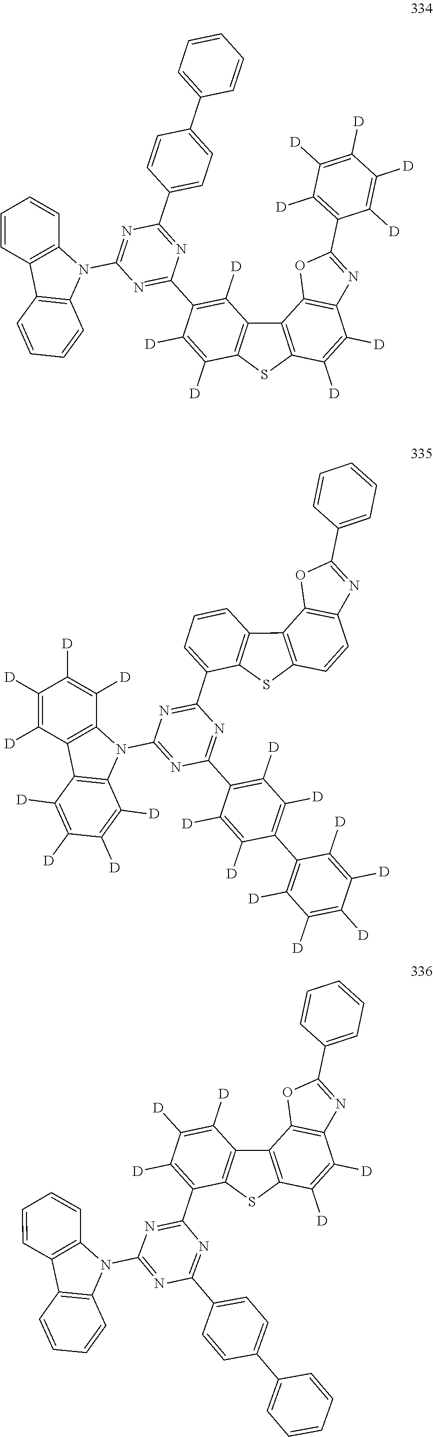

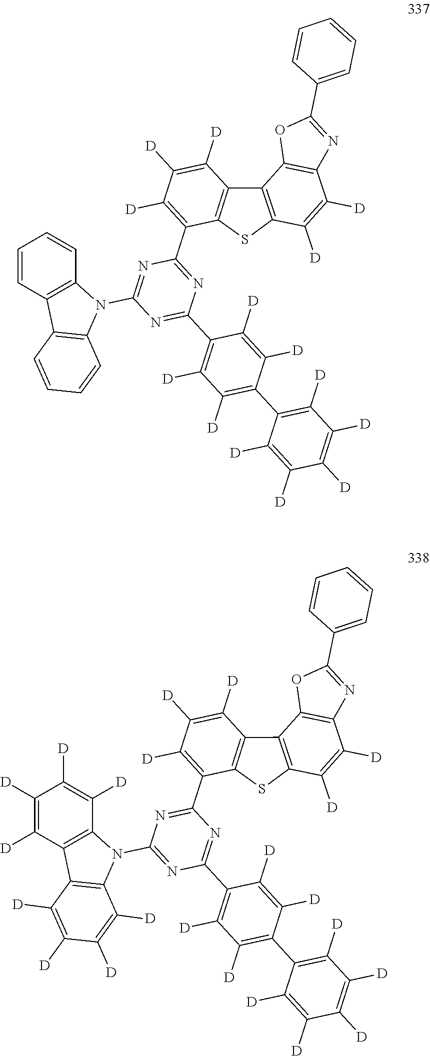 [SMILES: [2H]c1c([2H])c([2H])c(-c2c([2H])c([2H])c(-c3nc(-c4c([2H])c([2H])c([2H])c5c4sc4c([2H])c([2H])c6nc(-c7ccccc7)oc6c45)nc(-n4c5ccccc5c5ccccc54)n3)c([2H])c2[2H])c([2H])c1[2H] ; [2H]c1c([2H])c([2H])c(-c2c([2H])c([2H])c(-c3nc(-c4c([2H])c([2H])c([2H])c5c4sc4c([2H])c([2H])c6nc(-c7ccccc7)oc6c45)nc(-n4c5c([2H])c([2H])c([2H])c([2H])c5c5c([2H])c([2H])c([2H])c([2H])c54)n3)c([2H])c2[2H])c([2H])c1[2H]]

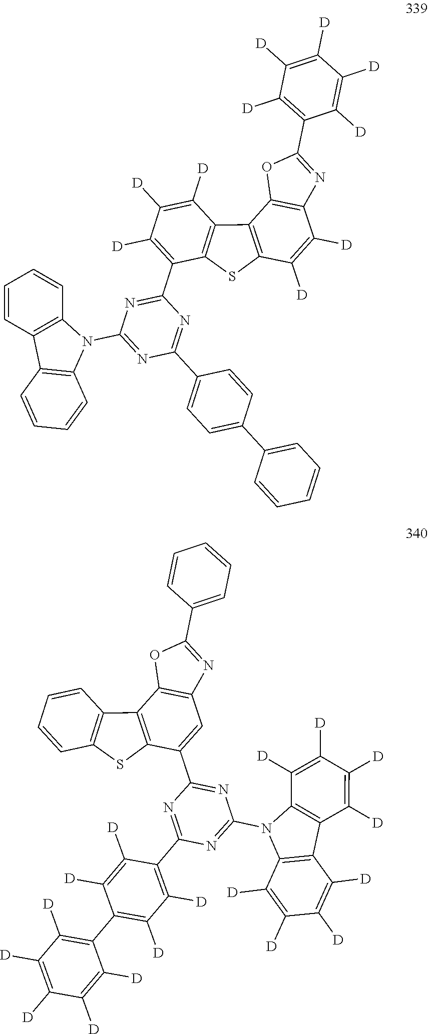 [SMILES: [2H]c1c([2H])c([2H])c(-c2nc3c([2H])c([2H])c4sc5c(-c6nc(-c7ccc(-c8ccccc8)cc7)nc(-n7c8ccccc8c8ccccc87)n6)c([2H])c([2H])c([2H])c5c4c3o2)c([2H])c1[2H] ; [2H]c1c([2H])c([2H])c(-c2c([2H])c([2H])c(-c3nc(-c4cc5nc(-c6ccccc6)oc5c5c4sc4ccccc45)nc(-n4c5c([2H])c([2H])c([2H])c([2H])c5c5c([2H])c([2H])c([2H])c([2H])c54)n3)c([2H])c2[2H])c([2H])c1[2H]]

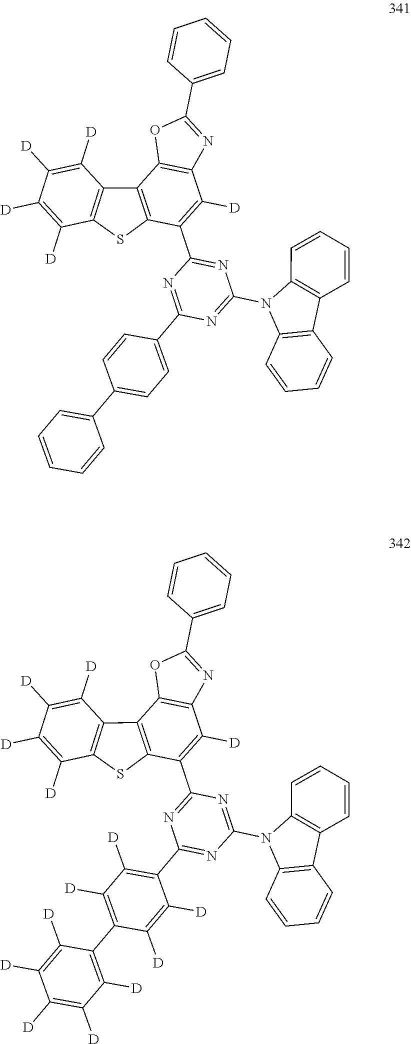 [SMILES: [2H]c1c([2H])c([2H])c2c(sc3c(-c4nc(-c5ccc(-c6ccccc6)cc5)nc(-n5c6ccccc6c6ccccc65)n4)c([2H])c4nc(-c5ccccc5)oc4c32)c1[2H] ; [2H]c1c([2H])c([2H])c(-c2c([2H])c([2H])c(-c3nc(-c4c([2H])c5nc(-c6ccccc6)oc5c5c4sc4c([2H])c([2H])c([2H])c([2H])c45)nc(-n4c5ccccc5c5ccccc54)n3)c([2H])c2[2H])c([2H])c1[2H]]

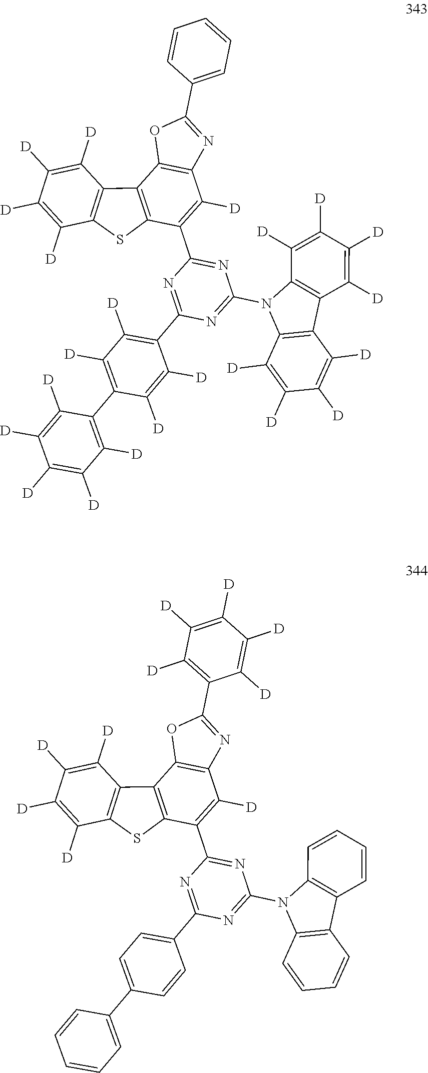 [SMILES: [2H]c1c([2H])c([2H])c(-c2c([2H])c([2H])c(-c3nc(-c4c([2H])c5nc(-c6ccccc6)oc5c5c4sc4c([2H])c([2H])c([2H])c([2H])c45)nc(-n4c5c([2H])c([2H])c([2H])c([2H])c5c5c([2H])c([2H])c([2H])c([2H])c54)n3)c([2H])c2[2H])c([2H])c1[2H] ; [2H]c1c([2H])c([2H])c(-c2nc3c([2H])c(-c4nc(-c5ccc(-c6ccccc6)cc5)nc(-n5c6ccccc6c6ccccc65)n4)c4sc5c([2H])c([2H])c([2H])c([2H])c5c4c3o2)c([2H])c1[2H]]

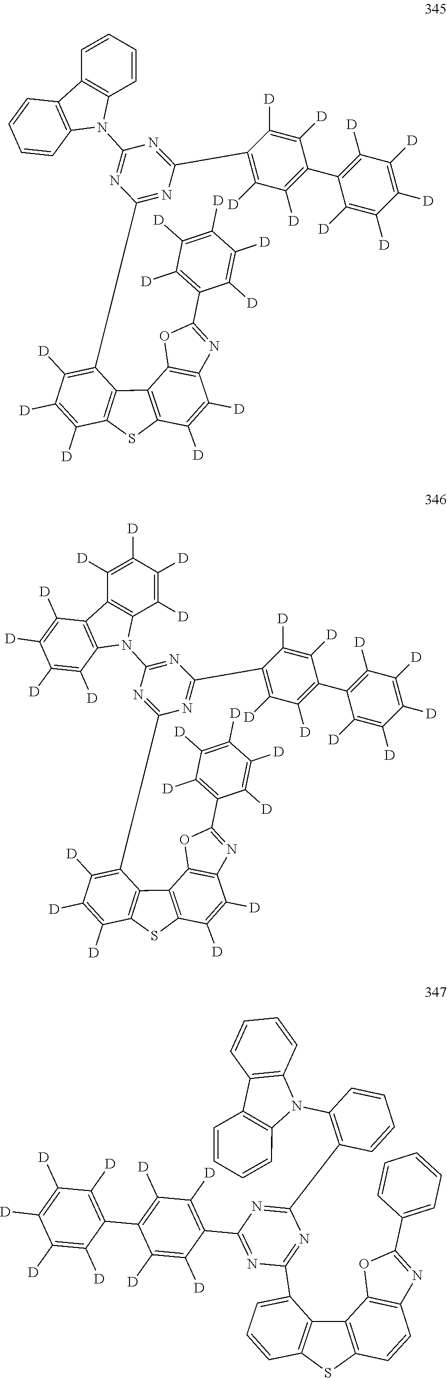

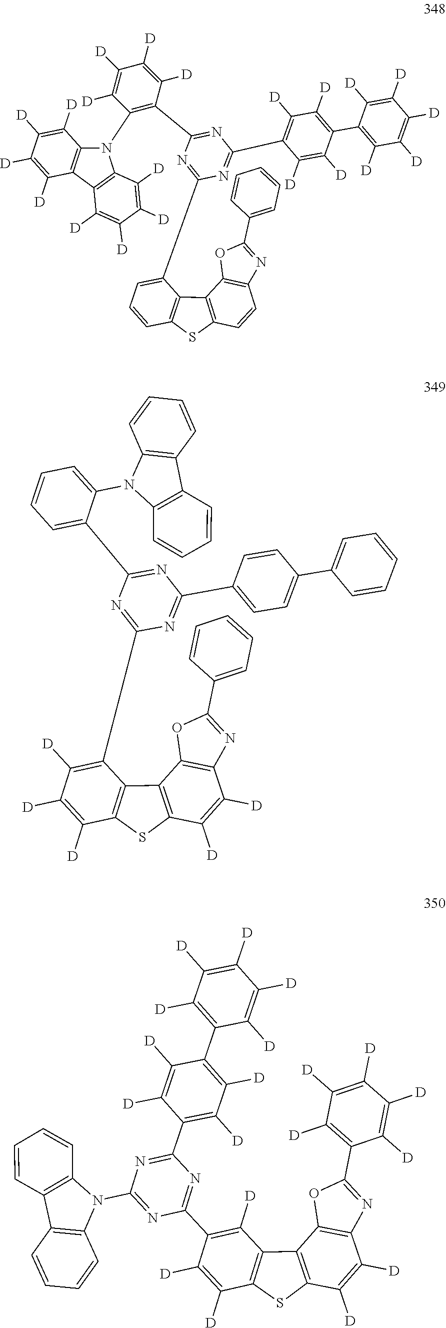

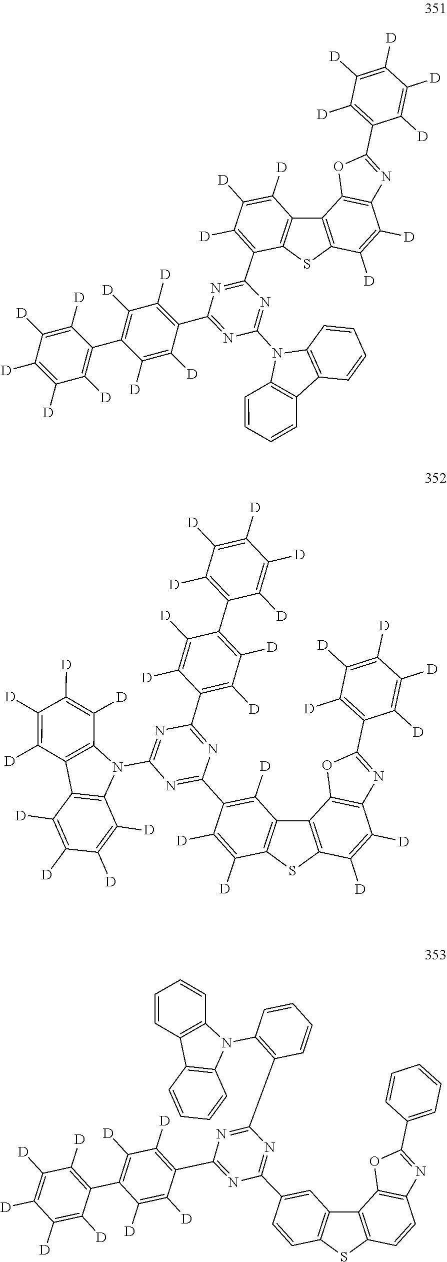

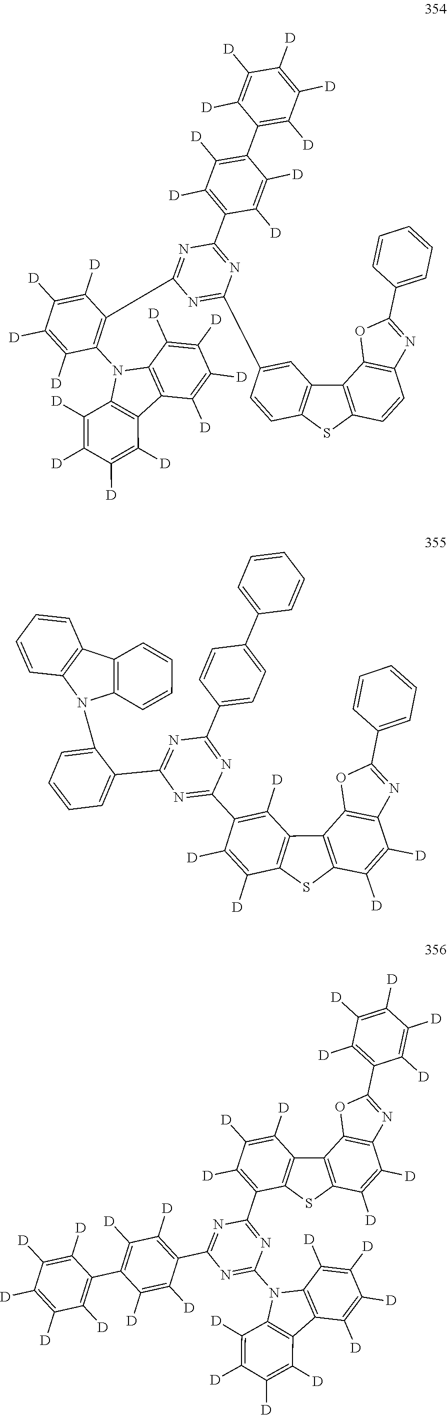

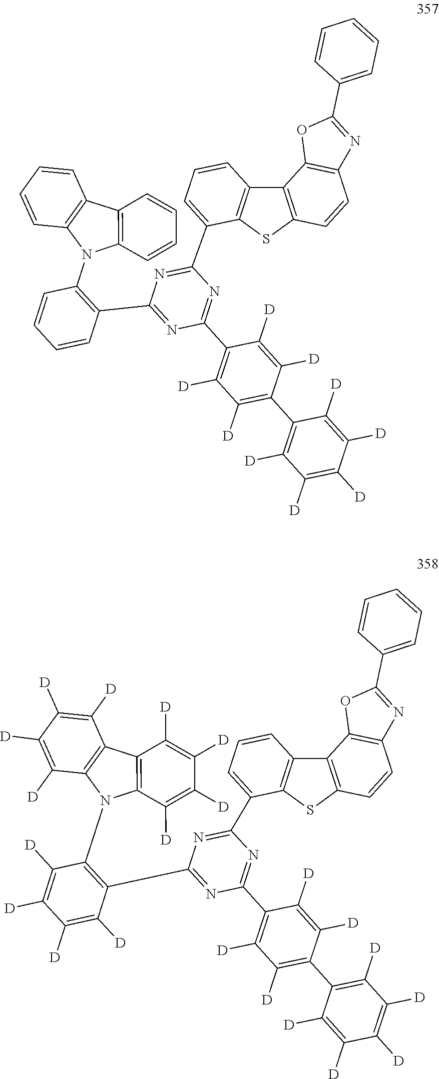 [SMILES: [2H]c1c([2H])c([2H])c(-c2c([2H])c([2H])c(-c3nc(-c4ccccc4-n4c5ccccc5c5ccccc54)nc(-c4cccc5c4sc4ccc6nc(-c7ccccc7)oc6c45)n3)c([2H])c2[2H])c([2H])c1[2H] ; [2H]c1c([2H])c([2H])c(-c2c([2H])c([2H])c(-c3nc(-c4c([2H])c([2H])c([2H])c([2H])c4-n4c5c([2H])c([2H])c([2H])c([2H])c5c5c([2H])c([2H])c([2H])c([2H])c54)nc(-c4cccc5c4sc4ccc6nc(-c7ccccc7)oc6c45)n3)c([2H])c2[2H])c([2H])c1[2H]]

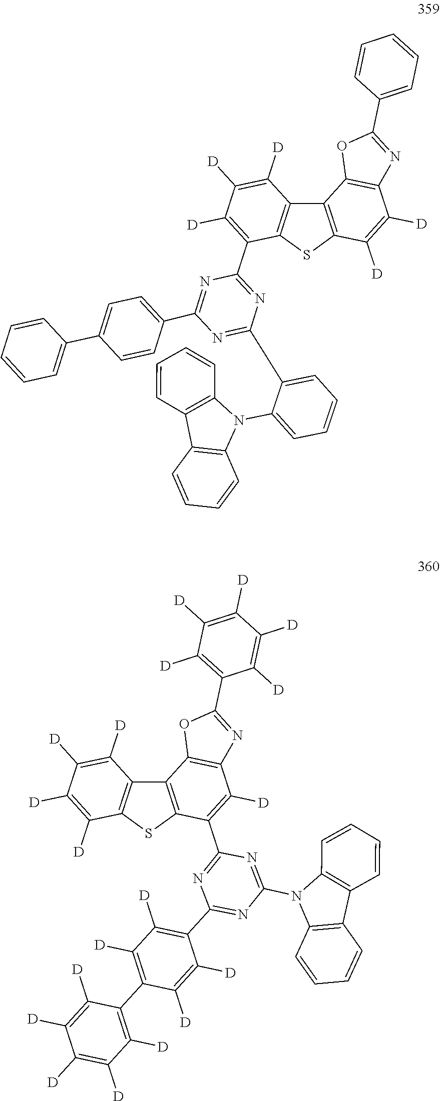 [SMILES: [2H]c1c([2H])c([2H])c2c(sc3c([2H])c([2H])c4nc(-c5ccccc5)oc4c32)c1-c1nc(-c2ccc(-c3ccccc3)cc2)nc(-c2ccccc2-n2c3ccccc3c3ccccc32)n1 ; [2H]c1c([2H])c([2H])c(-c2nc3c([2H])c(-c4nc(-c5c([2H])c([2H])c(-c6c([2H])c([2H])c([2H])c([2H])c6[2H])c([2H])c5[2H])nc(-n5c6ccccc6c6ccccc65)n4)c4sc5c([2H])c([2H])c([2H])c([2H])c5c4c3o2)c([2H])c1[2H]]

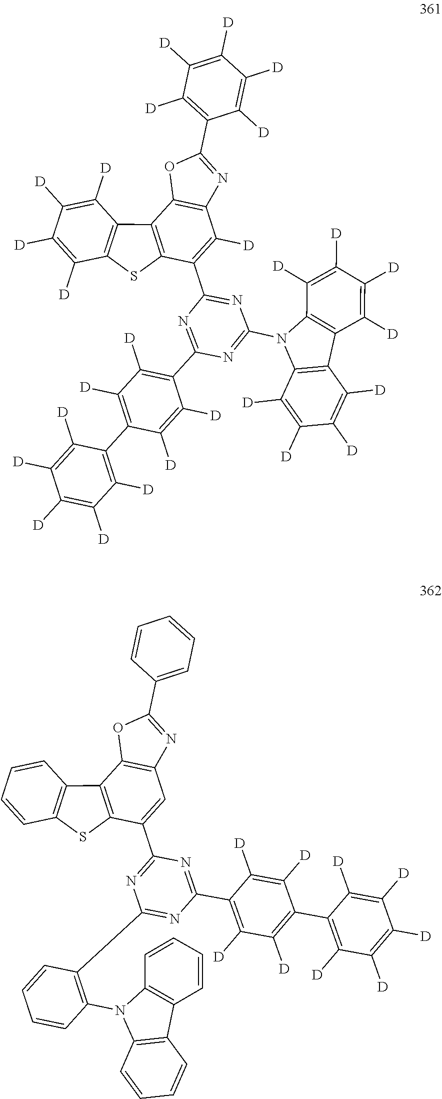 [SMILES: [2H]c1c([2H])c([2H])c(-c2nc3c([2H])c(-c4nc(-c5c([2H])c([2H])c(-c6c([2H])c([2H])c([2H])c([2H])c6[2H])c([2H])c5[2H])nc(-n5c6c([2H])c([2H])c([2H])c([2H])c6c6c([2H])c([2H])c([2H])c([2H])c65)n4)c4sc5c([2H])c([2H])c([2H])c([2H])c5c4c3o2)c([2H])c1[2H] ; [2H]c1c([2H])c([2H])c(-c2c([2H])c([2H])c(-c3nc(-c4ccccc4-n4c5ccccc5c5ccccc54)nc(-c4cc5nc(-c6ccccc6)oc5c5c4sc4ccccc45)n3)c([2H])c2[2H])c([2H])c1[2H]]

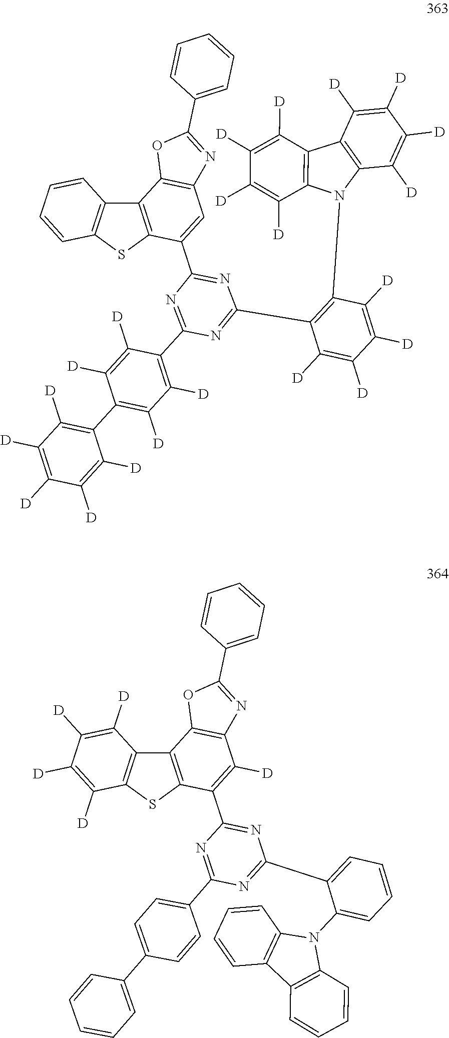 [SMILES: [2H]c1c([2H])c([2H])c(-c2c([2H])c([2H])c(-c3nc(-c4c([2H])c([2H])c([2H])c([2H])c4-n4c5c([2H])c([2H])c([2H])c([2H])c5c5c([2H])c([2H])c([2H])c([2H])c54)nc(-c4cc5nc(-c6ccccc6)oc5c5c4sc4ccccc45)n3)c([2H])c2[2H])c([2H])c1[2H] ; [2H]c1c([2H])c([2H])c2c(sc3c(-c4nc(-c5ccc(-c6ccccc6)cc5)nc(-c5ccccc5-n5c6ccccc6c6ccccc65)n4)c([2H])c4nc(-c5ccccc5)oc4c32)c1[2H]]

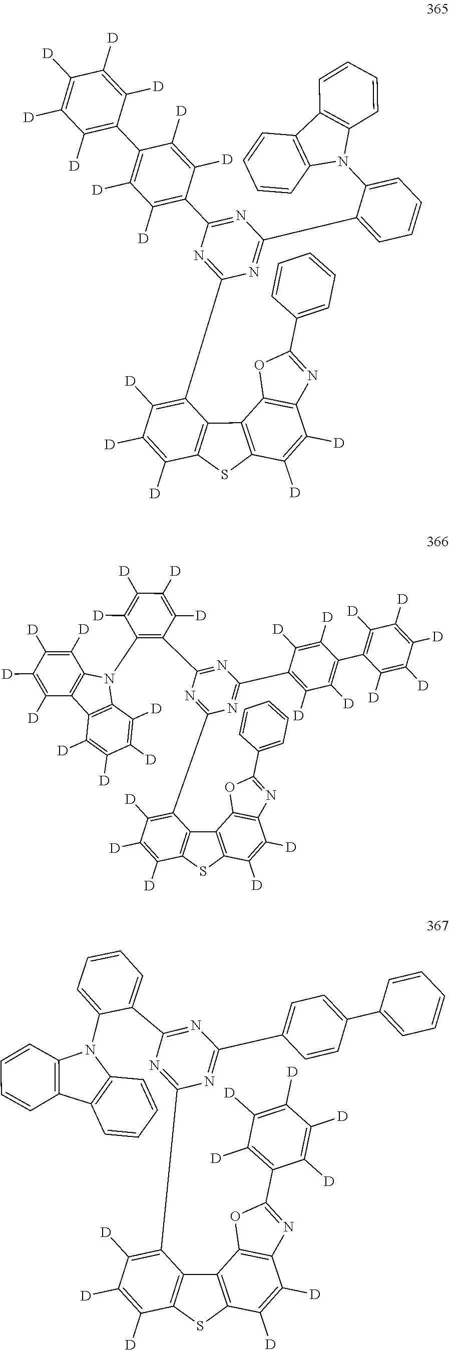

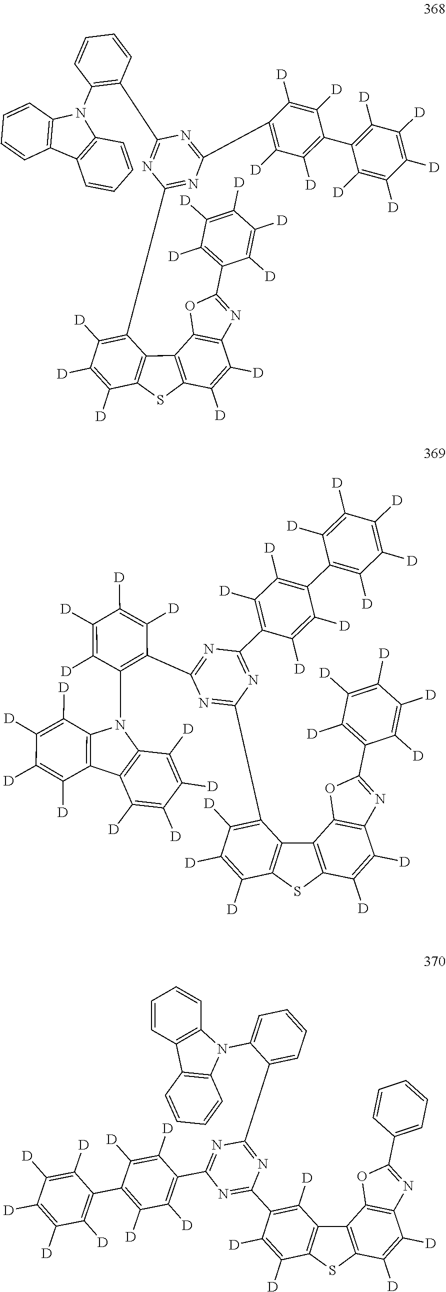

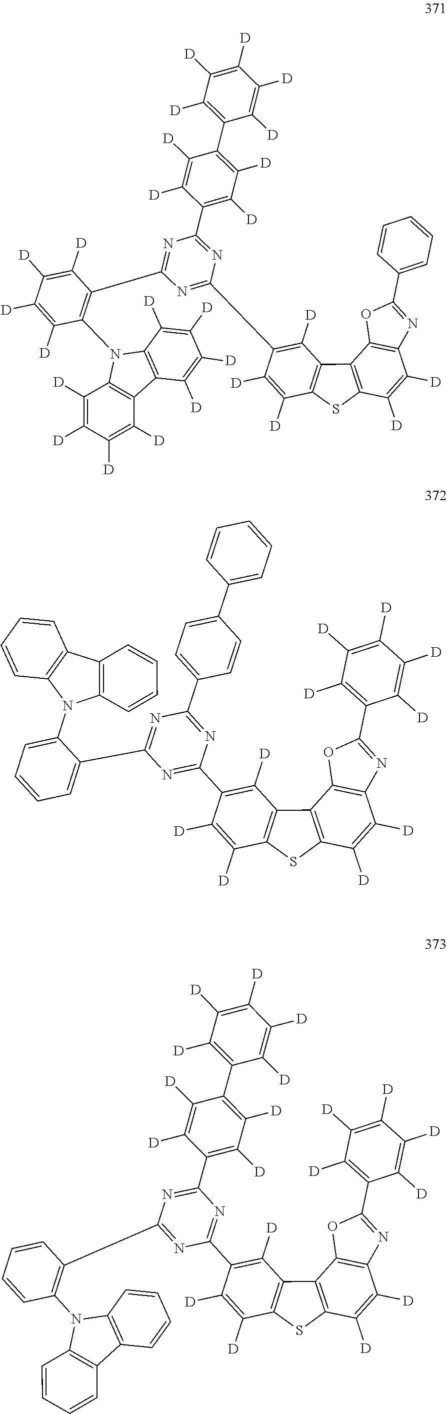

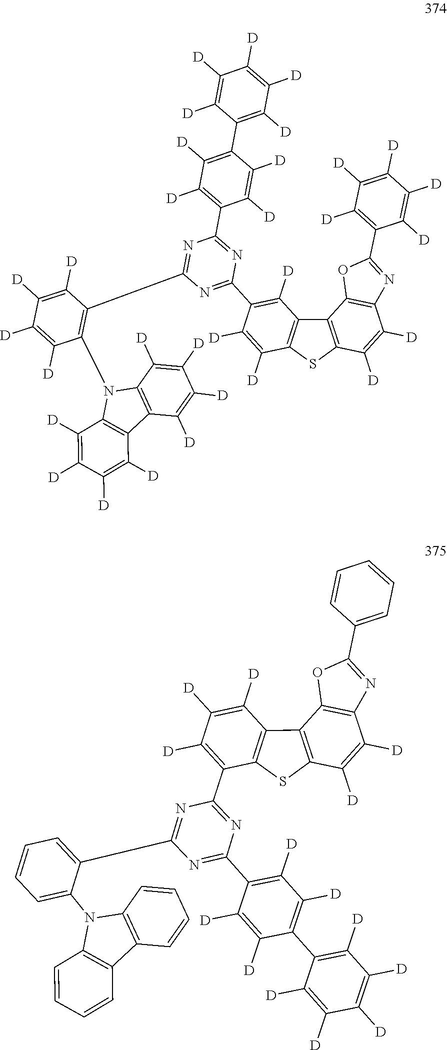 [SMILES: [2H]c1c([2H])c([2H])c(-c2nc3c([2H])c([2H])c4sc5c([2H])c([2H])c(-c6nc(-c7c([2H])c([2H])c(-c8c([2H])c([2H])c([2H])c([2H])c8[2H])c([2H])c7[2H])nc(-c7c([2H])c([2H])c([2H])c([2H])c7-n7c8c([2H])c([2H])c([2H])c([2H])c8c8c([2H])c([2H])c([2H])c([2H])c87)n6)c([2H])c5c4c3o2)c([2H])c1[2H] ; [2H]c1c([2H])c([2H])c(-c2c([2H])c([2H])c(-c3nc(-c4ccccc4-n4c5ccccc5c5ccccc54)nc(-c4c([2H])c([2H])c([2H])c5c4sc4c([2H])c([2H])c6nc(-c7ccccc7)oc6c45)n3)c([2H])c2[2H])c([2H])c1[2H]]

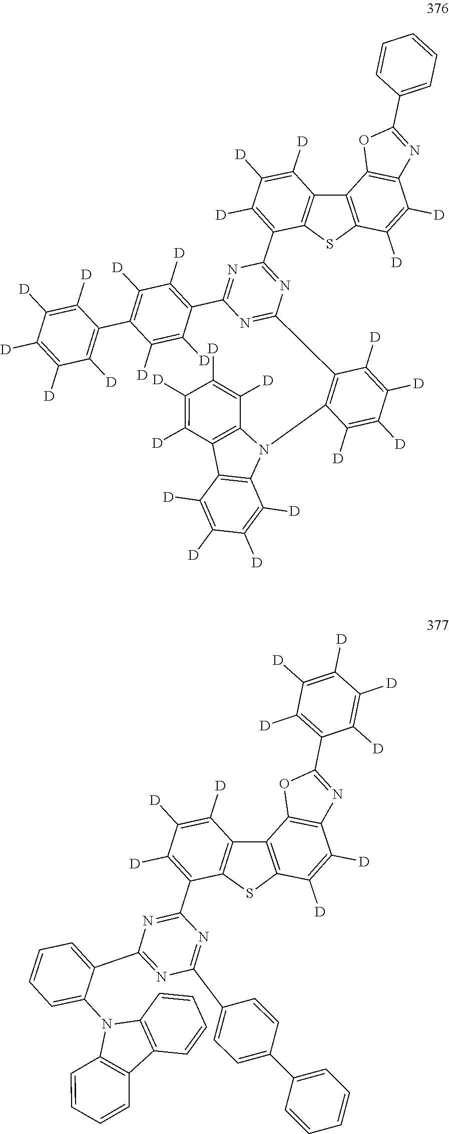 [SMILES: [2H]c1c([2H])c([2H])c(-c2c([2H])c([2H])c(-c3nc(-c4c([2H])c([2H])c([2H])c([2H])c4-n4c5c([2H])c([2H])c([2H])c([2H])c5c5c([2H])c([2H])c([2H])c([2H])c54)nc(-c4c([2H])c([2H])c([2H])c5c4sc4c([2H])c([2H])c6nc(-c7ccccc7)oc6c45)n3)c([2H])c2[2H])c([2H])c1[2H] ; [2H]c1c([2H])c([2H])c(-c2nc3c([2H])c([2H])c4sc5c(-c6nc(-c7ccc(-c8ccccc8)cc7)nc(-c7ccccc7-n7c8ccccc8c8ccccc87)n6)c([2H])c([2H])c([2H])c5c4c3o2)c([2H])c1[2H]]

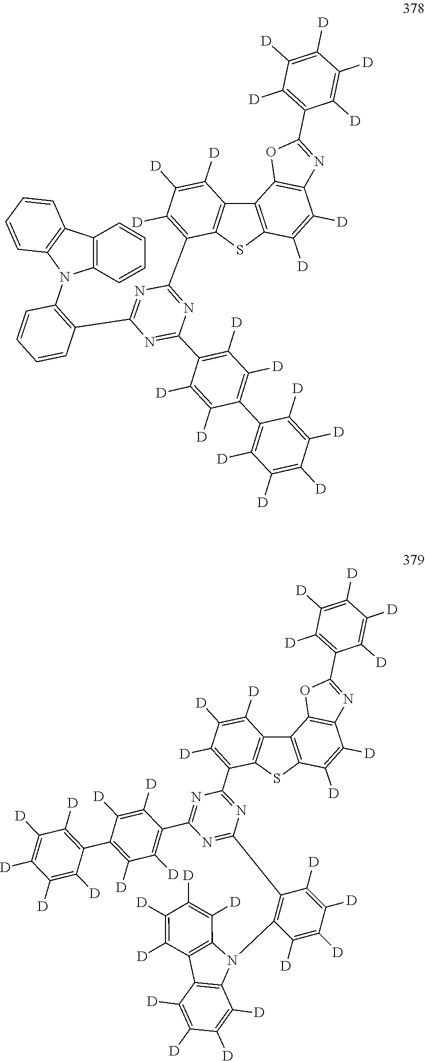 [SMILES: [2H]c1c([2H])c([2H])c(-c2nc3c([2H])c([2H])c4sc5c(-c6nc(-c7ccccc7-n7c8ccccc8c8ccccc87)nc(-c7c([2H])c([2H])c(-c8c([2H])c([2H])c([2H])c([2H])c8[2H])c([2H])c7[2H])n6)c([2H])c([2H])c([2H])c5c4c3o2)c([2H])c1[2H] ; [2H]c1c([2H])c([2H])c(-c2nc3c([2H])c([2H])c4sc5c(-c6nc(-c7c([2H])c([2H])c(-c8c([2H])c([2H])c([2H])c([2H])c8[2H])c([2H])c7[2H])nc(-c7c([2H])c([2H])c([2H])c([2H])c7-n7c8c([2H])c([2H])c([2H])c([2H])c8c8c([2H])c([2H])c([2H])c([2H])c87)n6)c([2H])c([2H])c([2H])c5c4c3o2)c([2H])c1[2H]]

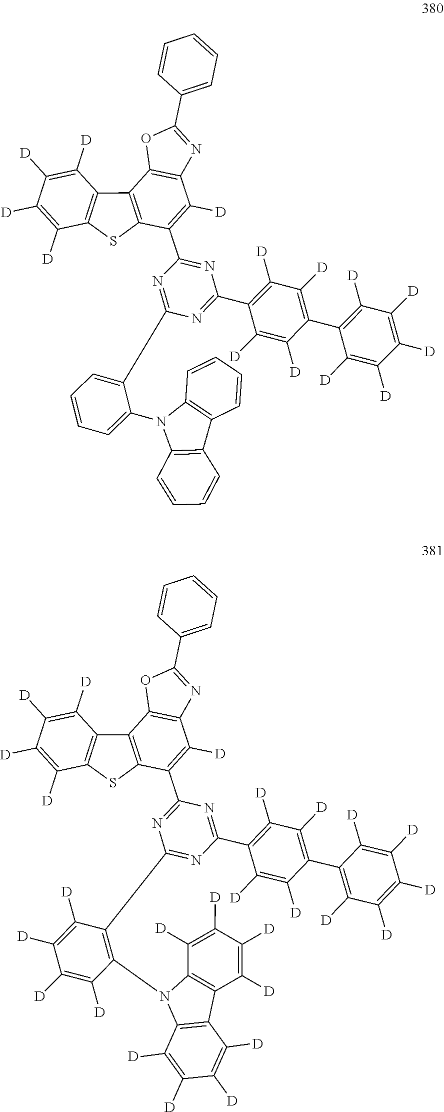 [SMILES: [2H]c1c([2H])c([2H])c(-c2c([2H])c([2H])c(-c3nc(-c4ccccc4-n4c5ccccc5c5ccccc54)nc(-c4c([2H])c5nc(-c6ccccc6)oc5c5c4sc4c([2H])c([2H])c([2H])c([2H])c45)n3)c([2H])c2[2H])c([2H])c1[2H] ; [2H]c1c([2H])c([2H])c(-c2c([2H])c([2H])c(-c3nc(-c4c([2H])c([2H])c([2H])c([2H])c4-n4c5c([2H])c([2H])c([2H])c([2H])c5c5c([2H])c([2H])c([2H])c([2H])c54)nc(-c4c([2H])c5nc(-c6ccccc6)oc5c5c4sc4c([2H])c([2H])c([2H])c([2H])c45)n3)c([2H])c2[2H])c([2H])c1[2H]]

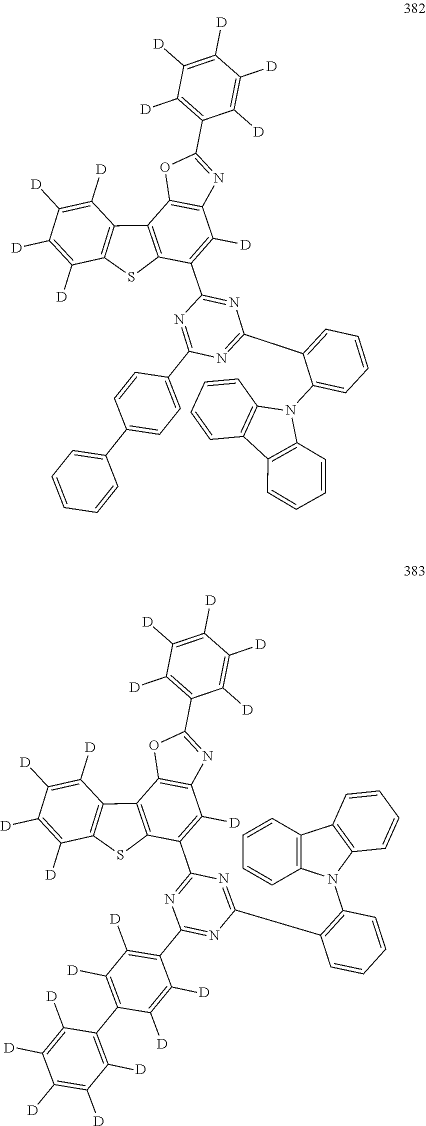 [SMILES: [2H]c1c([2H])c([2H])c(-c2nc3c([2H])c(-c4nc(-c5ccc(-c6ccccc6)cc5)nc(-c5ccccc5-n5c6ccccc6c6ccccc65)n4)c4sc5c([2H])c([2H])c([2H])c([2H])c5c4c3o2)c([2H])c1[2H] ; [2H]c1c([2H])c([2H])c(-c2nc3c([2H])c(-c4nc(-c5ccccc5-n5c6ccccc6c6ccccc65)nc(-c5c([2H])c([2H])c(-c6c([2H])c([2H])c([2H])c([2H])c6[2H])c([2H])c5[2H])n4)c4sc5c([2H])c([2H])c([2H])c([2H])c5c4c3o2)c([2H])c1[2H]]

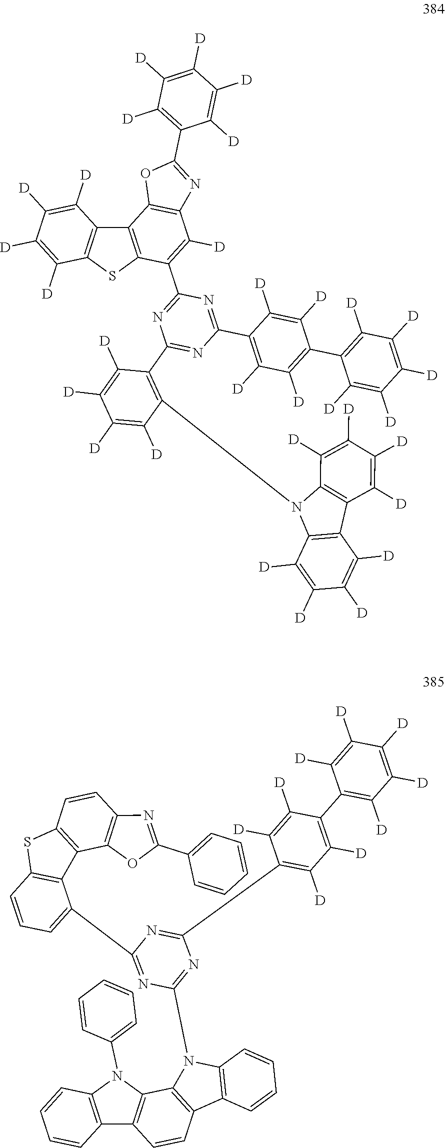 [SMILES: [2H]c1c([2H])c([2H])c(-c2nc3c([2H])c(-c4nc(-c5c([2H])c([2H])c(-c6c([2H])c([2H])c([2H])c([2H])c6[2H])c([2H])c5[2H])nc(-c5c([2H])c([2H])c([2H])c([2H])c5-n5c6c([2H])c([2H])c([2H])c([2H])c6c6c([2H])c([2H])c([2H])c([2H])c65)n4)c4sc5c([2H])c([2H])c([2H])c([2H])c5c4c3o2)c([2H])c1[2H] ; [2H]c1c([2H])c([2H])c(-c2c([2H])c([2H])c(-c3nc(-c4cccc5sc6ccc7nc(-c8ccccc8)oc7c6c45)nc(-n4c5ccccc5c5ccc6c7ccccc7n(-c7ccccc7)c6c54)n3)c([2H])c2[2H])c([2H])c1[2H]]

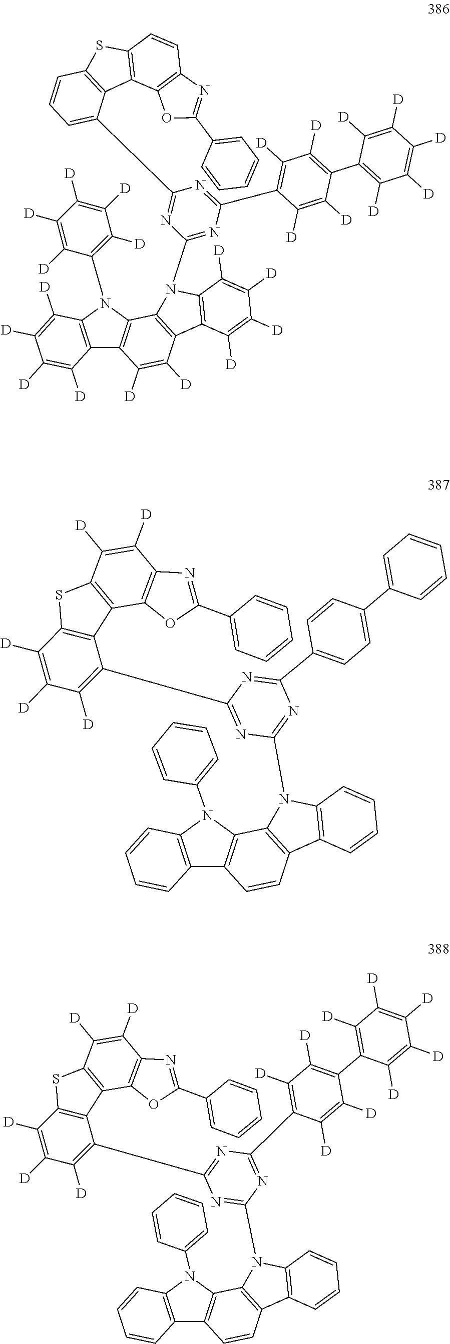

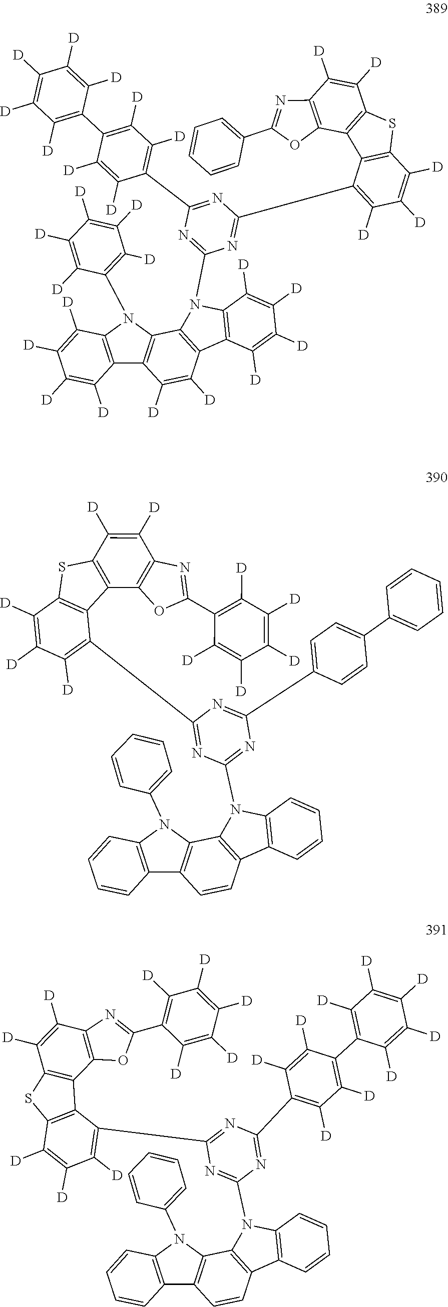

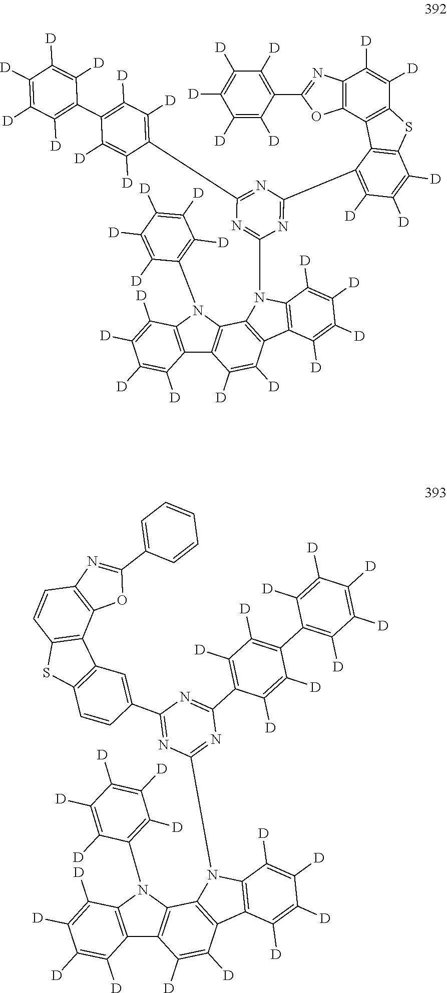

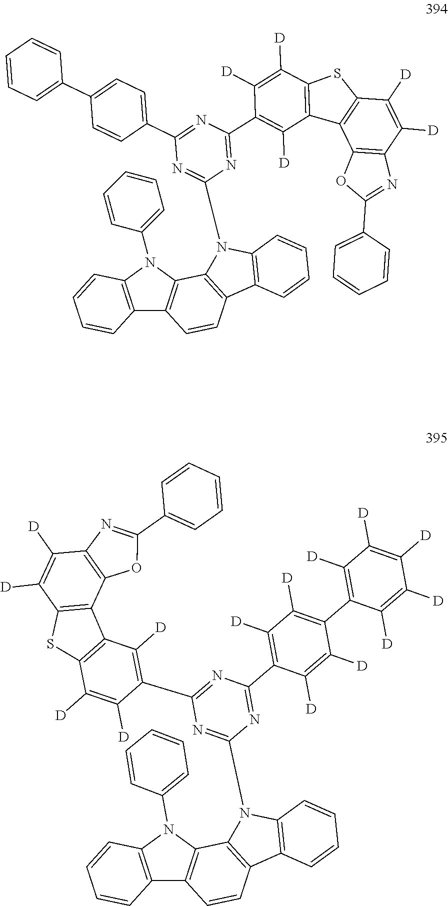 [SMILES: [2H]c1c(-c2nc(-c3ccc(-c4ccccc4)cc3)nc(-n3c4ccccc4c4ccc5c6ccccc6n(-c6ccccc6)c5c43)n2)c([2H])c2c(sc3c([2H])c([2H])c4nc(-c5ccccc5)oc4c32)c1[2H] ; [2H]c1c([2H])c([2H])c(-c2c([2H])c([2H])c(-c3nc(-c4c([2H])c([2H])c5sc6c([2H])c([2H])c7nc(-c8ccccc8)oc7c6c5c4[2H])nc(-n4c5ccccc5c5ccc6c7ccccc7n(-c7ccccc7)c6c54)n3)c([2H])c2[2H])c([2H])c1[2H]]

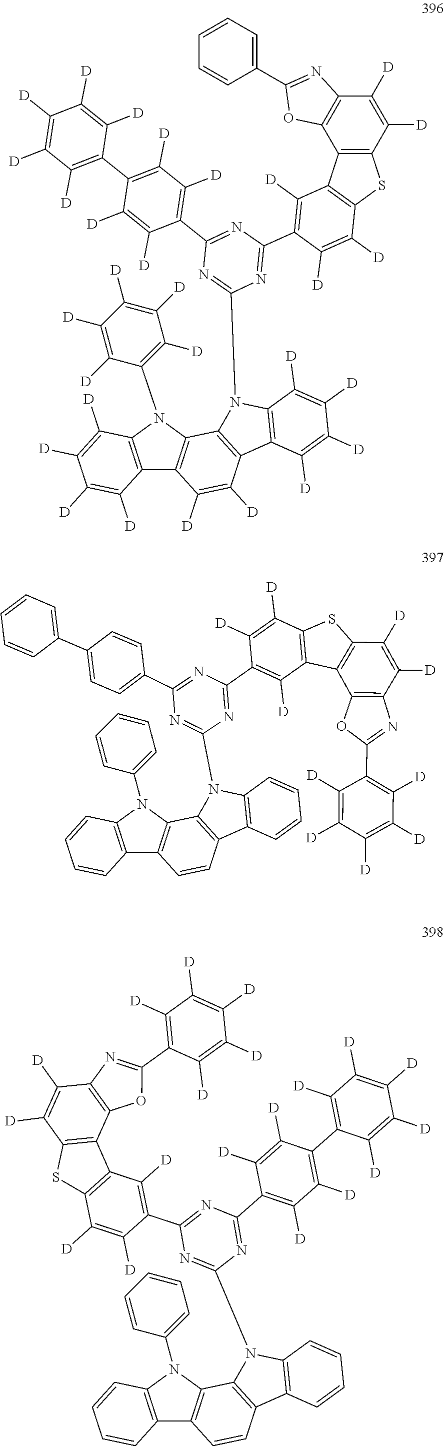

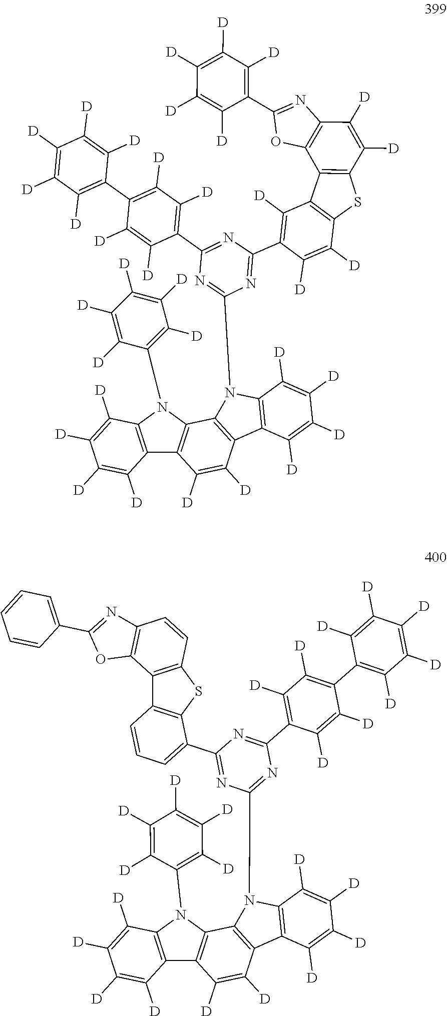

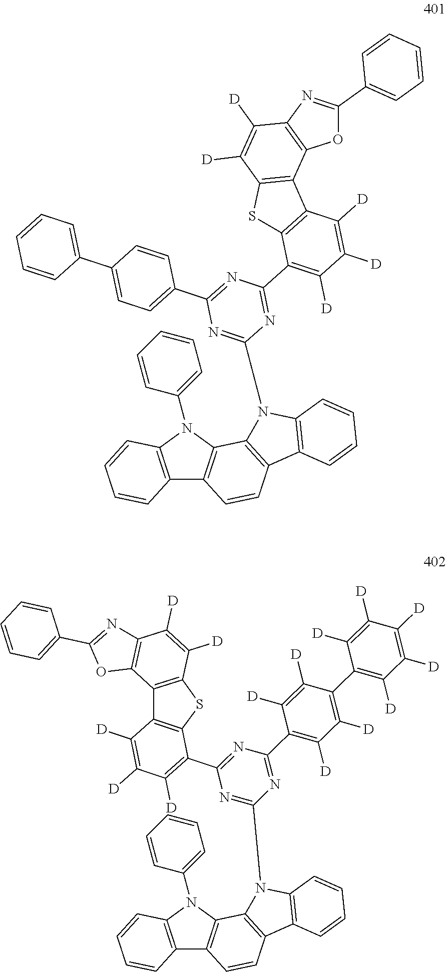 [SMILES: [2H]c1c([2H])c([2H])c2c(sc3c([2H])c([2H])c4nc(-c5ccccc5)oc4c32)c1-c1nc(-c2ccc(-c3ccccc3)cc2)nc(-n2c3ccccc3c3ccc4c5ccccc5n(-c5ccccc5)c4c32)n1 ; [2H]c1c([2H])c([2H])c(-c2c([2H])c([2H])c(-c3nc(-c4c([2H])c([2H])c([2H])c5c4sc4c([2H])c([2H])c6nc(-c7ccccc7)oc6c45)nc(-n4c5ccccc5c5ccc6c7ccccc7n(-c7ccccc7)c6c54)n3)c([2H])c2[2H])c([2H])c1[2H]]

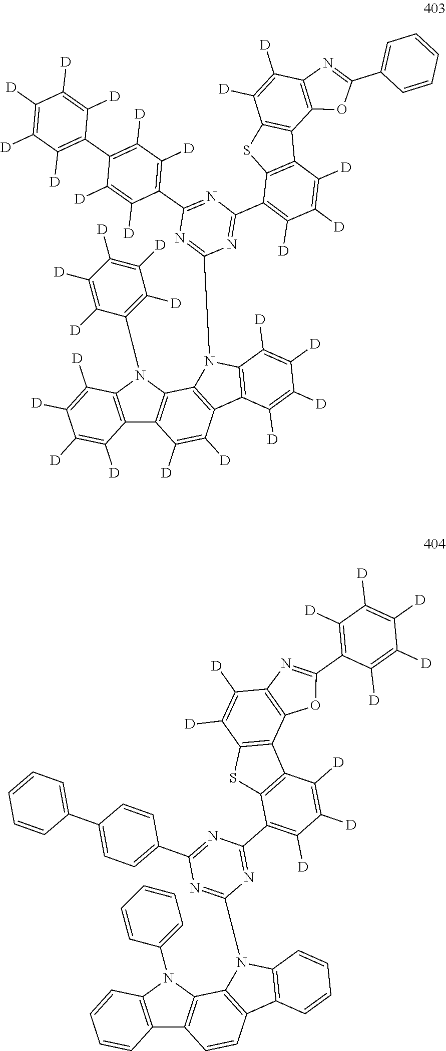 [SMILES: [2H]c1c([2H])c([2H])c(-c2c([2H])c([2H])c(-c3nc(-c4c([2H])c([2H])c([2H])c5c4sc4c([2H])c([2H])c6nc(-c7ccccc7)oc6c45)nc(-n4c5c([2H])c([2H])c([2H])c([2H])c5c5c([2H])c([2H])c6c7c([2H])c([2H])c([2H])c([2H])c7n(-c7c([2H])c([2H])c([2H])c([2H])c7[2H])c6c54)n3)c([2H])c2[2H])c([2H])c1[2H] ; [2H]c1c([2H])c([2H])c(-c2nc3c([2H])c([2H])c4sc5c(-c6nc(-c7ccc(-c8ccccc8)cc7)nc(-n7c8ccccc8c8ccc9c%10ccccc%10n(-c%10ccccc%10)c9c87)n6)c([2H])c([2H])c([2H])c5c4c3o2)c([2H])c1[2H]]

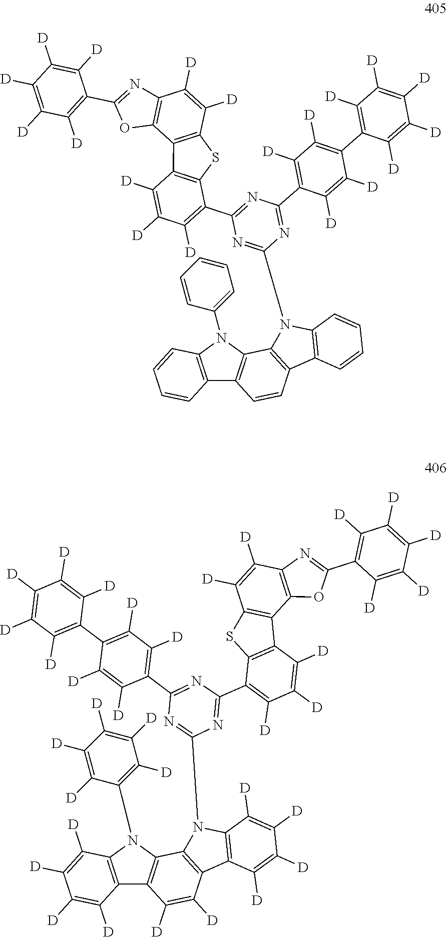

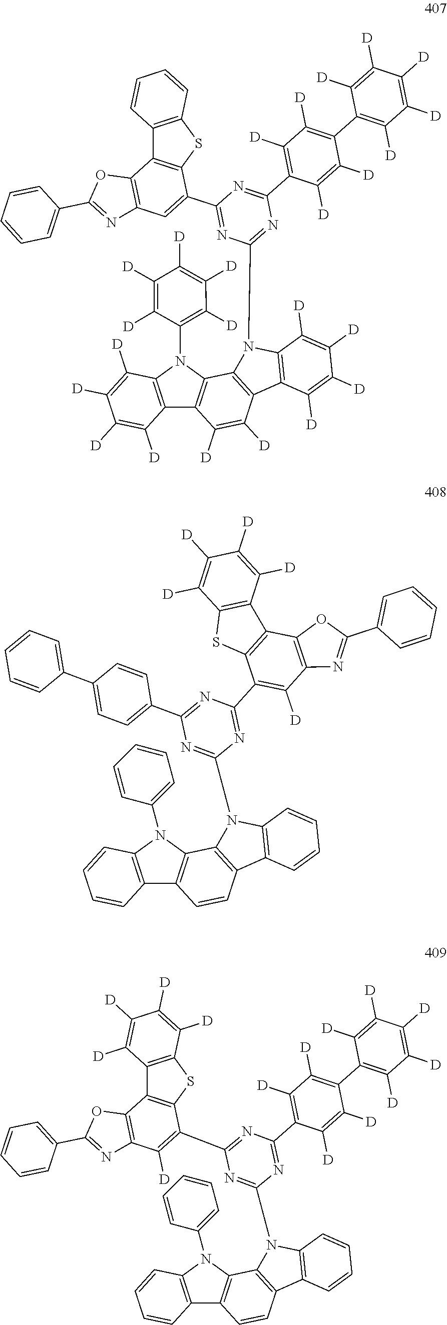

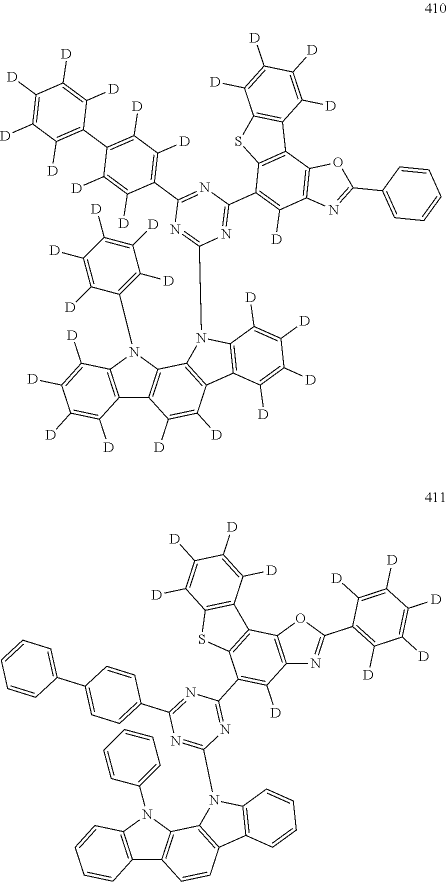 [SMILES: [2H]c1c([2H])c([2H])c(-c2c([2H])c([2H])c(-c3nc(-c4c([2H])c5nc(-c6ccccc6)oc5c5c4sc4c([2H])c([2H])c([2H])c([2H])c45)nc(-n4c5c([2H])c([2H])c([2H])c([2H])c5c5c([2H])c([2H])c6c7c([2H])c([2H])c([2H])c([2H])c7n(-c7c([2H])c([2H])c([2H])c([2H])c7[2H])c6c54)n3)c([2H])c2[2H])c([2H])c1[2H] ; [2H]c1c([2H])c([2H])c(-c2nc3c([2H])c(-c4nc(-c5ccc(-c6ccccc6)cc5)nc(-n5c6ccccc6c6ccc7c8ccccc8n(-c8ccccc8)c7c65)n4)c4sc5c([2H])c([2H])c([2H])c([2H])c5c4c3o2)c([2H])c1[2H]]

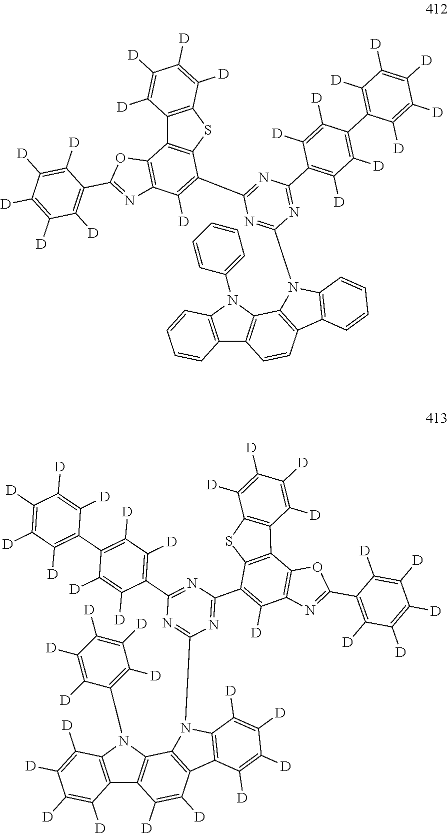

In addition to the aforementioned compound for the organic optoelectronic device, one or more compounds may be further included.

For example, a dopant may be further included.

The dopant may be, for example, a phosphorescent dopant, for example, a red, green or blue phosphorescent dopant, and may be, for example, a red or green phosphorescent dopant.

The dopant is a material mixed with the compound for the organic optoelectronic device in a small amount to cause light emission, and may be generally a material such as a metal complex that emits light by multiple excitation into a triplet or more. The dopant may be, for example, an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example, a compound represented by Chemical Formula Z, but is not limited thereto.

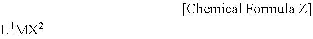

In Chemical Formula Z, M is a metal, and L¹ and X² are the same or different, and are a ligand to form a complex compound with M.

The M may be for example Ir, Pt, Os, Ti, Zr, Hf, Eu, Tb, Tm, Fe, Co, Ni, Ru, Rh, Pd, or a combination thereof and the L¹ and X² may be, for example, a bidentate ligand.

Examples of ligands represented by L¹ and X² may be selected from the chemical formulas listed in Group A, but are not limited thereto.

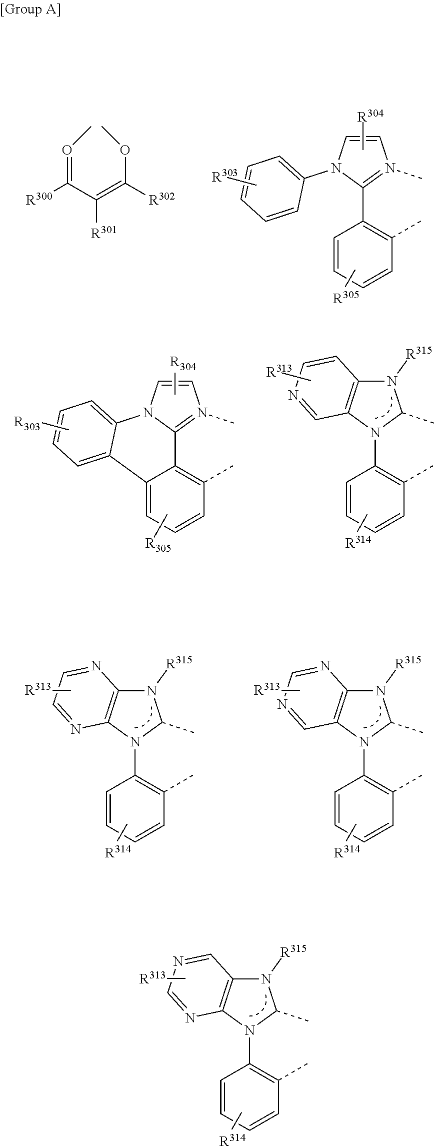

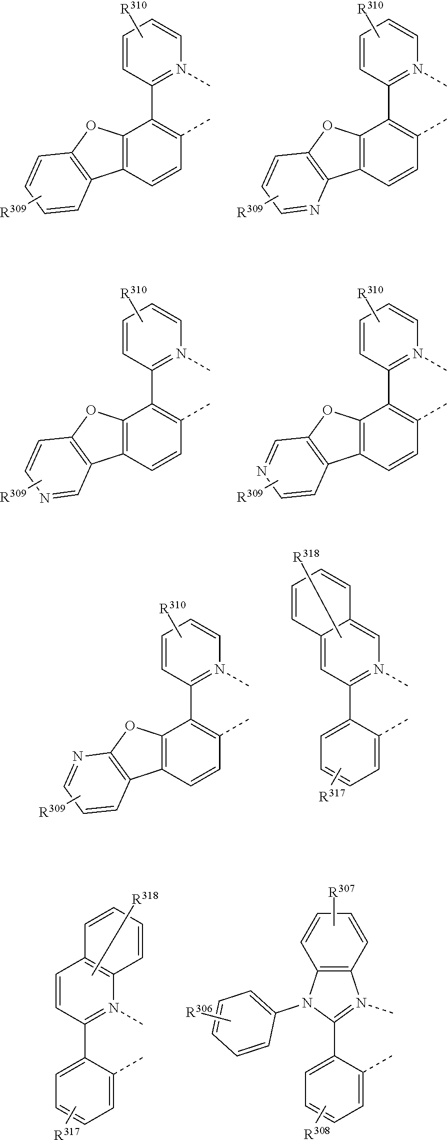

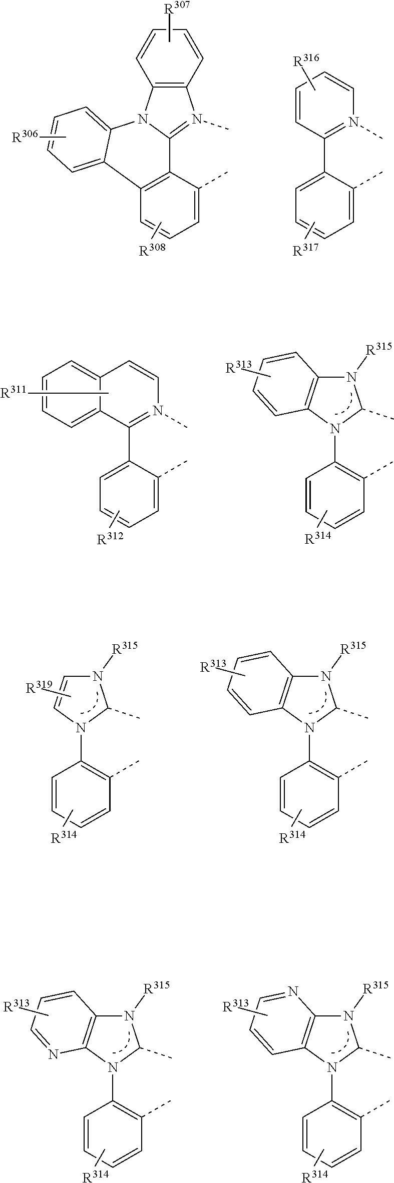

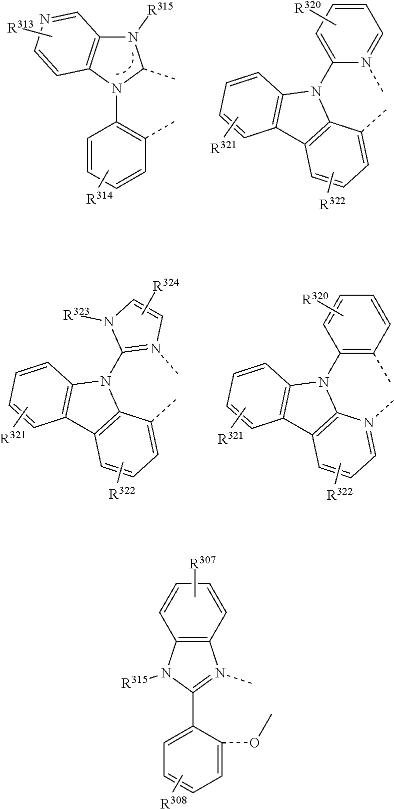

In Group A,
R³⁰⁰ to R³⁰² are each independently hydrogen, deuterium, a C1 to C30 alkyl group substituted or unsubstituted with halogen, a C6 to C30 aryl group substituted or unsubstituted with C1 to C30 alkyl or a halogen, and
R³⁰³ to R³²⁴ are each independently hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

For example, a dopant represented by Chemical Formula V may be further included.

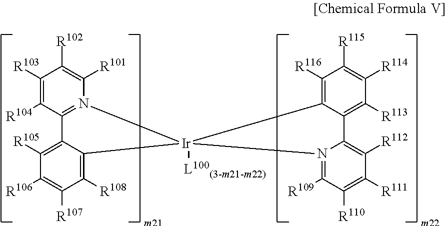

In Chemical Formula V,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a C1 to C6 alkyl group,
at least one of R¹⁰¹ to R¹¹⁶ is a functional group represented by Chemical Formula V-1,
L¹⁰⁰ is a bidentate ligand of a monovalent anion, and is a ligand that coordinates to iridium through a lone pair of carbons or heteroatoms, and
m21 and m22 are each independently any one of integers of 0 to 3, and m21+m22 is any one of integers of 1 to 3,

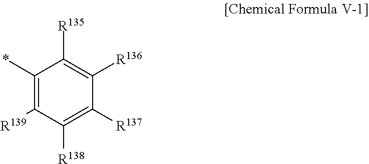

wherein, in Chemical Formula V-1,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and
* refers to a portion linked to a carbon atom.

For example, a dopant represented by Chemical Formula Z-1 may be further included.

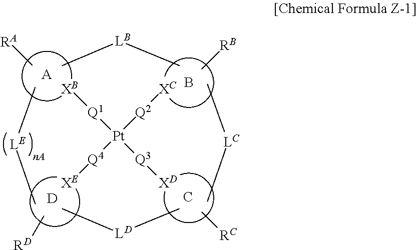

In Chemical Formula Z-1, rings A, B, C, and D are each independently a 5-membered or 6-membered carbocyclic or heterocyclic ring;
R^{A}, R^{B}, R^{C}, and R^{D} are each independently mono-, di-, tri-, or tetra-substitution, or unsubstitution;
L^{B}, L^{C}, and L^{D} are each independently a direct bond, BR, NR, PR, O, S, Se, C═O, S═O, SO₂, CRR′, SiRR′, GeRR′, or a combination thereof,
when nA is 1, L^{E} may be a direct bond, BR, NR, PR, O, S, Se, C═O, S═O, SO₂, CRR′, SiRR′, GeRR′, or a combination thereof; and when nA is 0, L^{E} does not exist;
R^{A}, R^{B}, R^{C}, R^{D}, R, and R′ are each independently hydrogen, deuterium, a halogen, alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

According to one embodiment, the dopant may be a platinum complex, and may be represented by, for example, Chemical Formula VI.

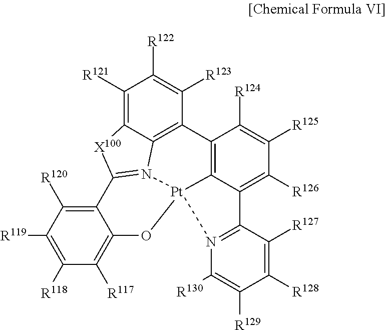

In Chemical Formula VI,
X¹⁰⁰ is selected from O, S, and NR¹³¹,
R¹¹⁷ to R¹³¹ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a C1 to C6 alkyl group,
at least one of R¹¹⁷ to R¹³¹ is —SiR¹³²R¹³³R¹³⁴ or a tert-butyl group, and
R¹³² to R¹³⁴ are each independently a C1 to C6 alkyl group.

Hereinafter, an organic optoelectronic device including the aforementioned compound for the organic optoelectronic device is described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

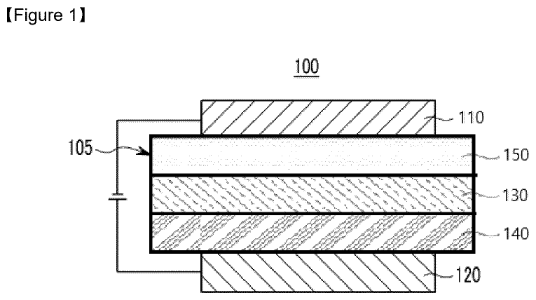

FIG. 1 is a cross-sectional view showing an organic light emitting diode according to an embodiment.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, LiF/Al, and BaF₂/Ca, but is not limited thereto.

The organic layer 105 may include the aforementioned compound for an organic optoelectronic device.

The organic layer 105 may include a light emitting layer 130, and the light emitting layer 130 may include the aforementioned compound for the organic optoelectronic device.

The composition for the organic optoelectronic device further including a dopant may be, for example, a green light emitting composition.

The light emitting layer 130 may include, for example, the aforementioned compound for the organic optoelectronic device as a phosphorescent host.

The organic layer may further include a charge transport region in addition to the light emitting layer.

The charge transport region may be, for example, a hole transport region 140.

The hole transport region 140 can further increase hole injection and/or hole mobility and block electrons between the anode 120 and the light emitting layer 130.

Specifically, the hole transport region 140 may include a hole transport layer between the anode 120 and the light emitting layer 130, and a hole transport auxiliary layer between the light emitting layer 130 and the hole transport layer, and at least one of the compounds listed in Group B may be included in at least one layer of the hole transport layer and the hole transport auxiliary layer.

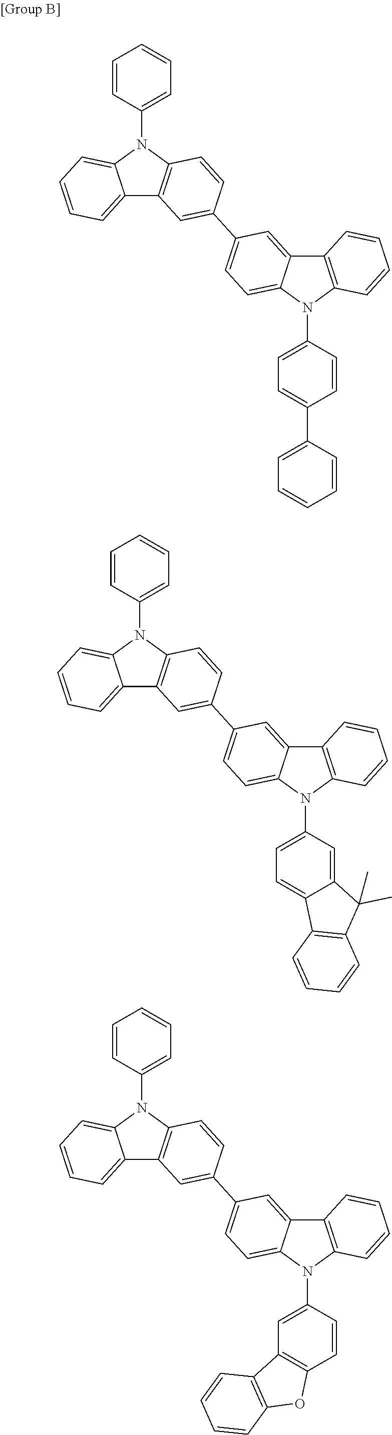 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

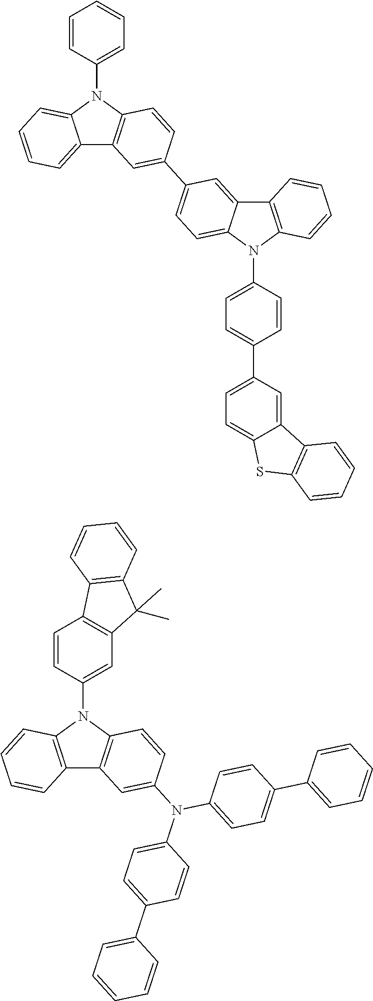 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

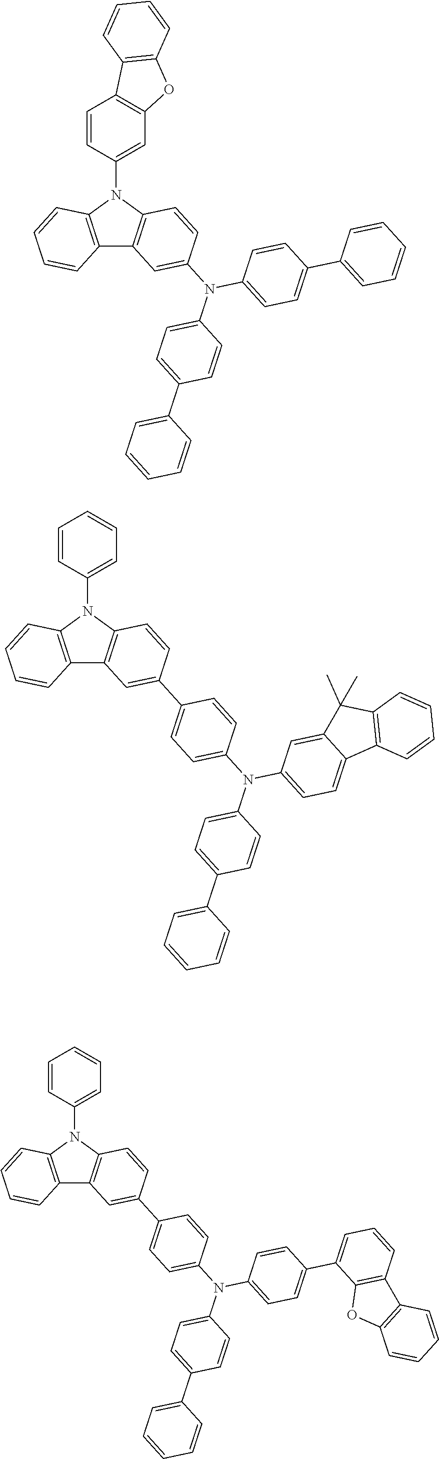 [SMILES: c1ccc(-c2ccc(N(c3ccc(-c4ccccc4)cc3)c3ccc4c(c3)c3ccccc3n4-c3ccc4c(c3)oc3ccccc34)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

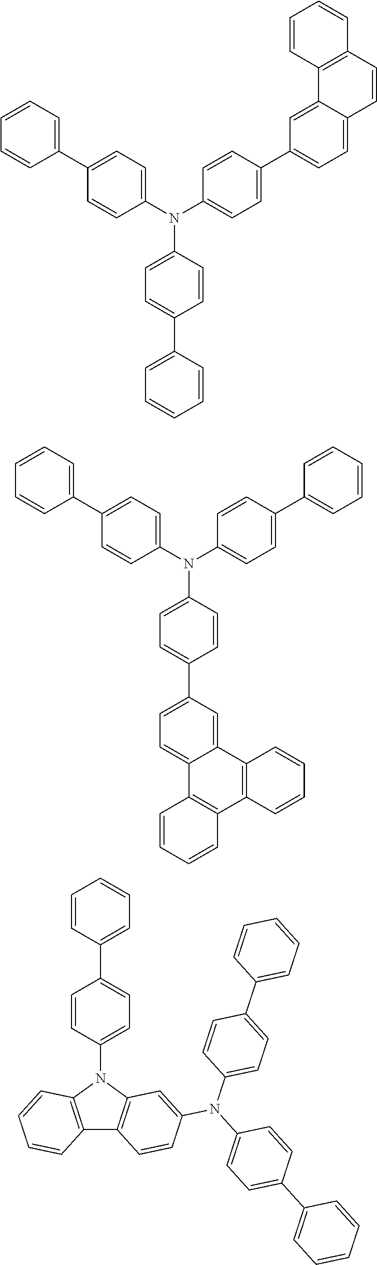 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1]

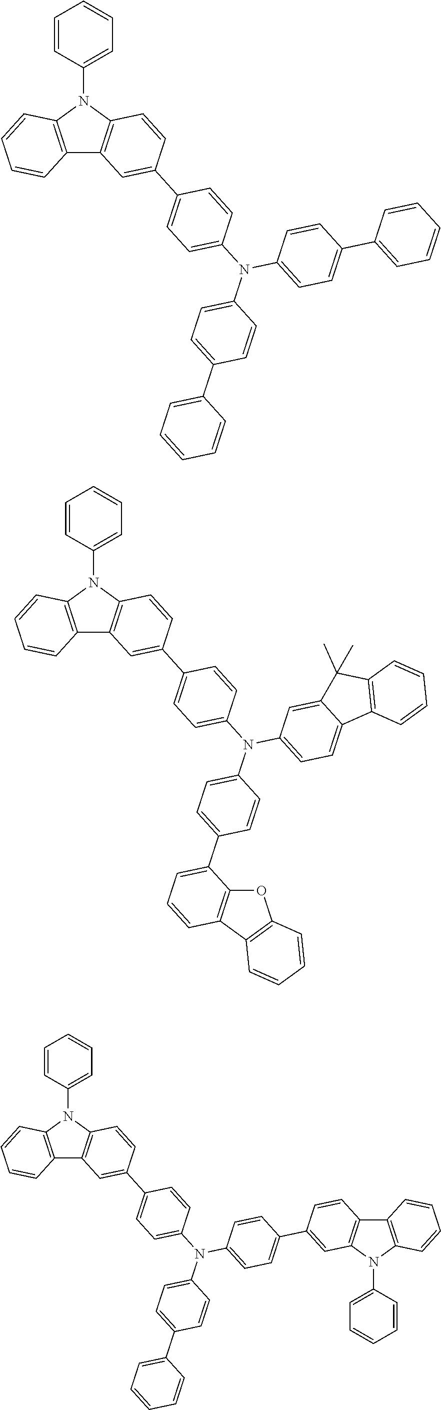 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

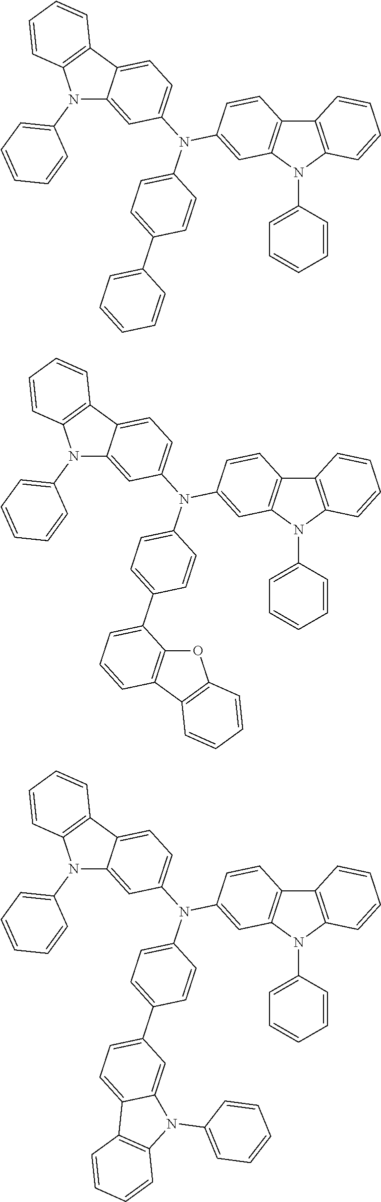 [SMILES: c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1]

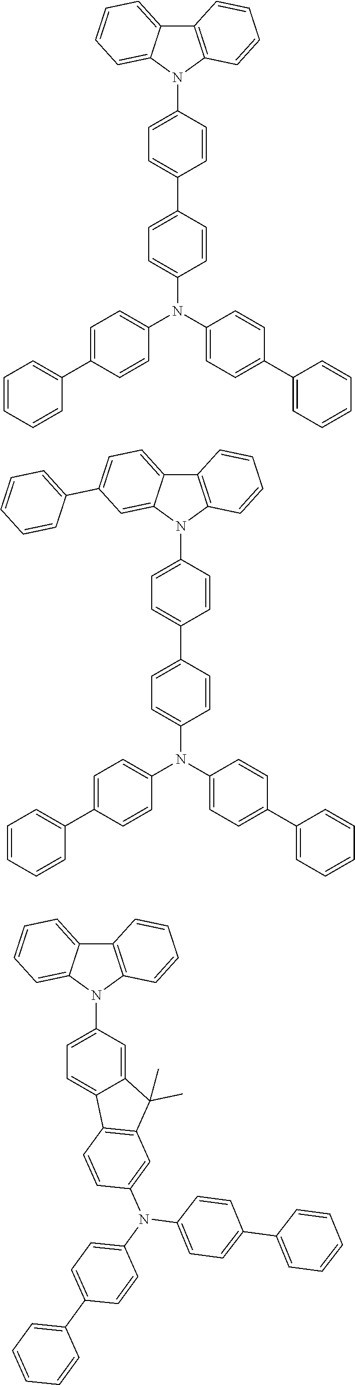

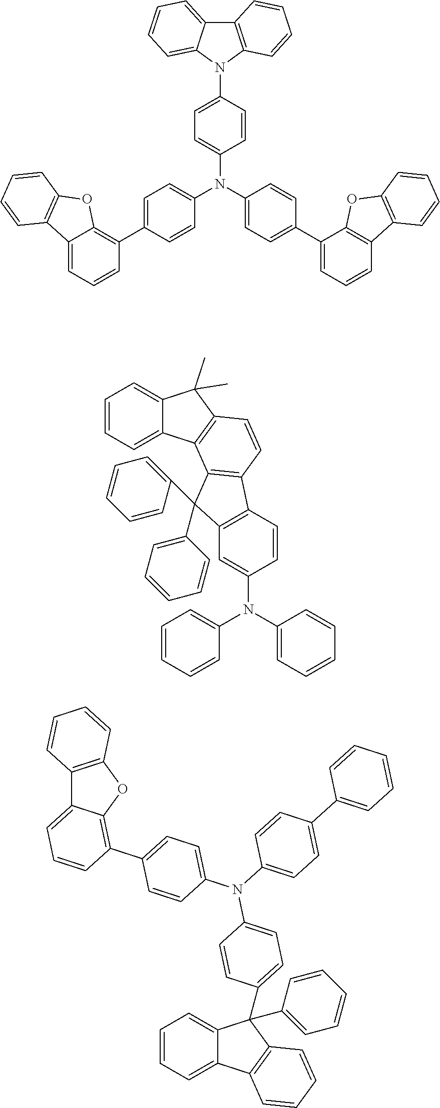 [SMILES: c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1]

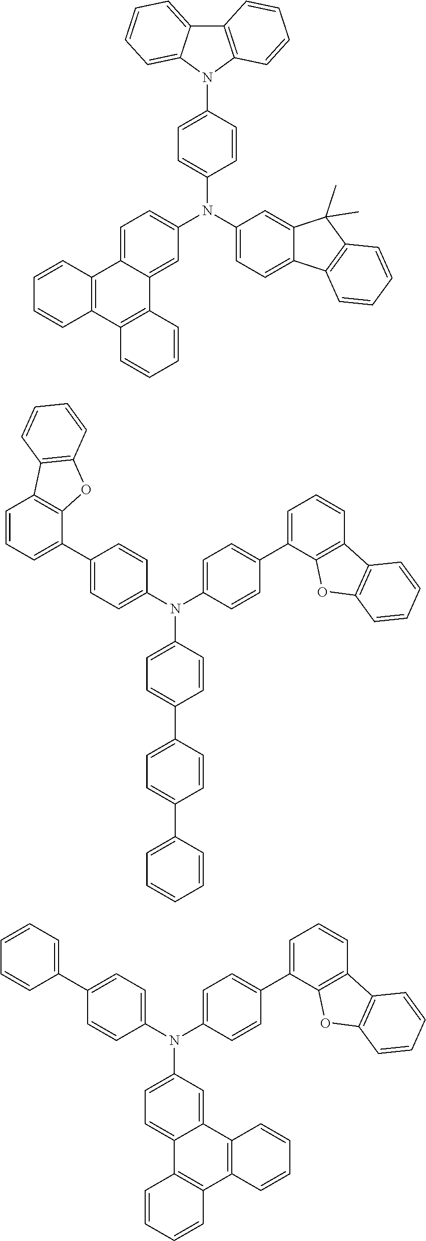 [SMILES: CC1(C)c2ccccc2-c2ccc(N(c3ccc(-n4c5ccccc5c5ccccc54)cc3)c3ccc4c5ccccc5c5ccccc5c4c3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1]

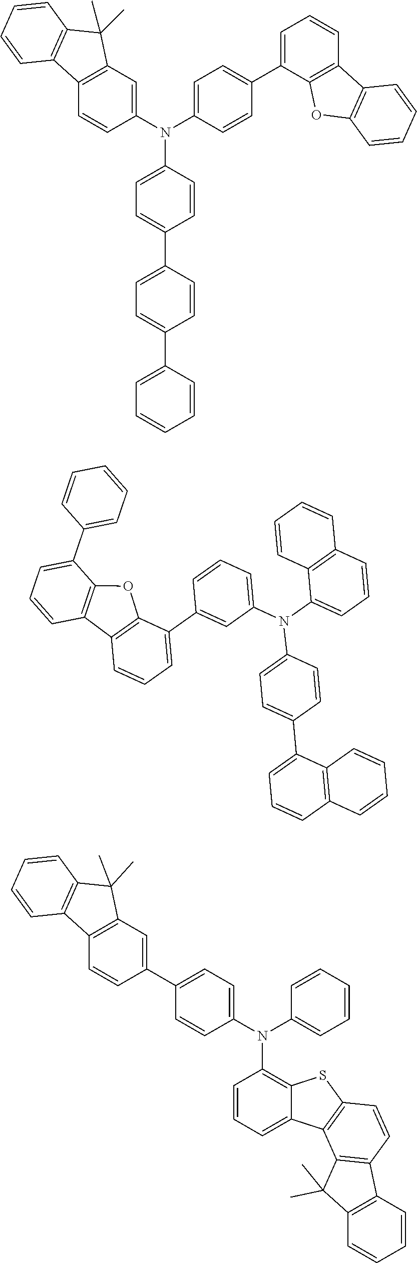 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21]

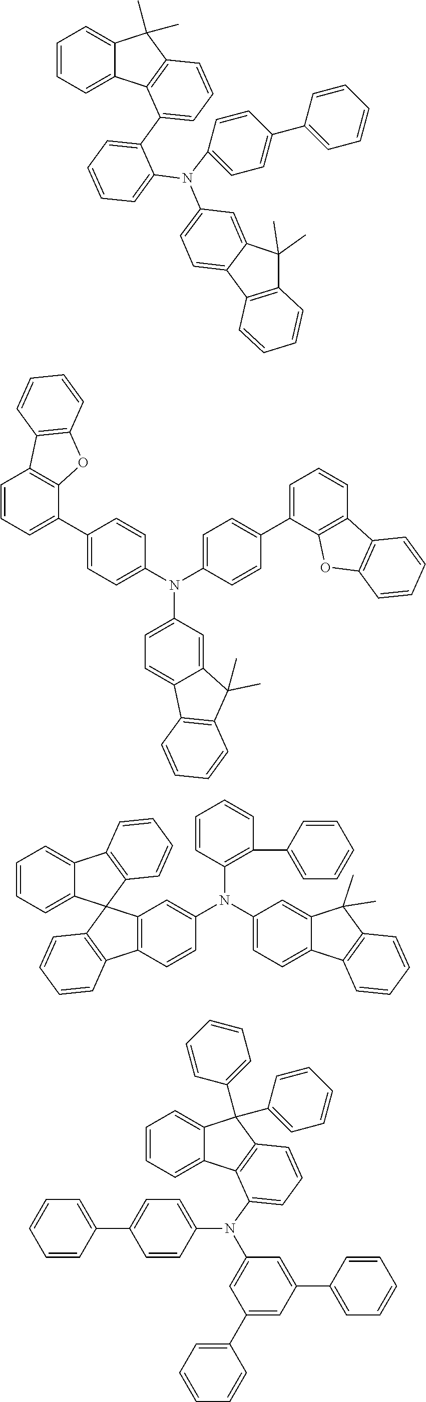

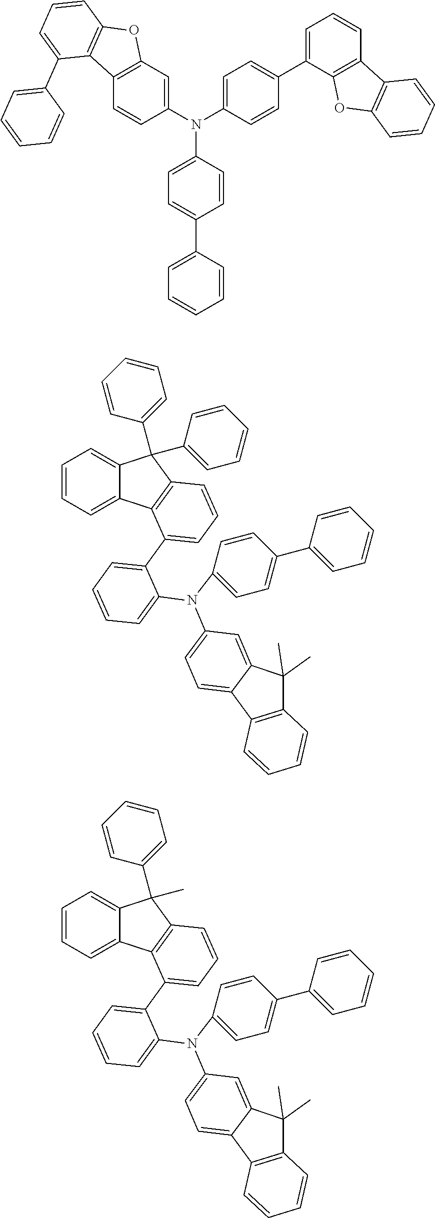 [SMILES: c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21]

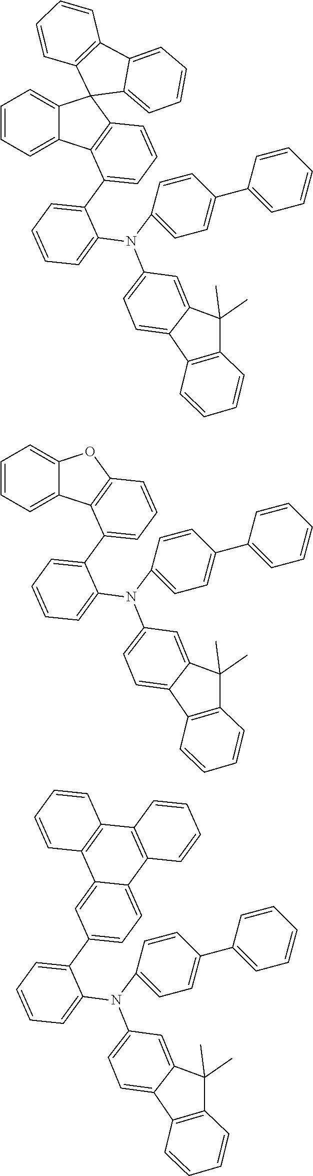

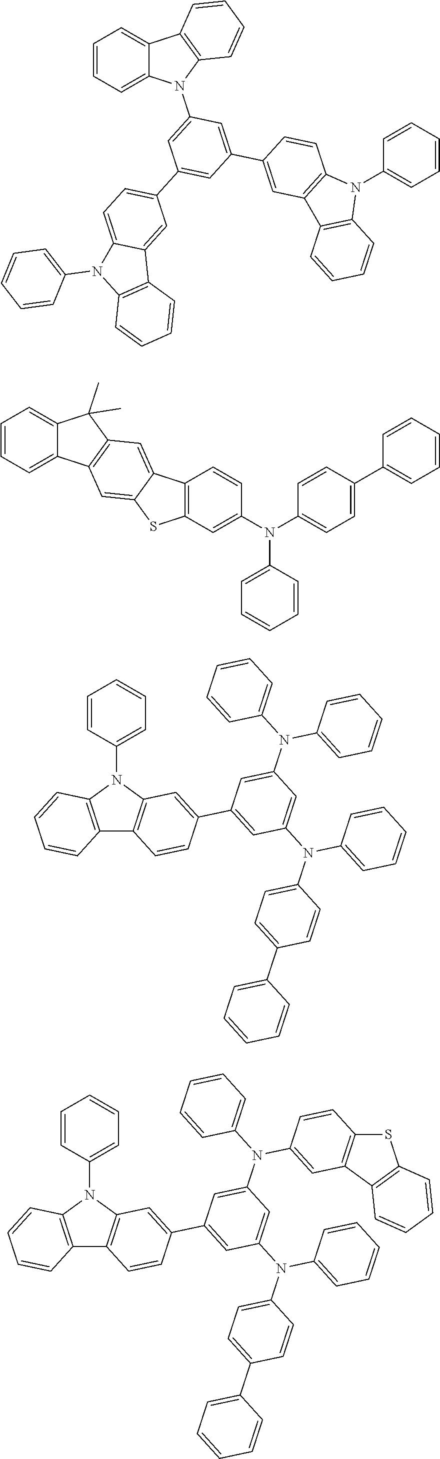

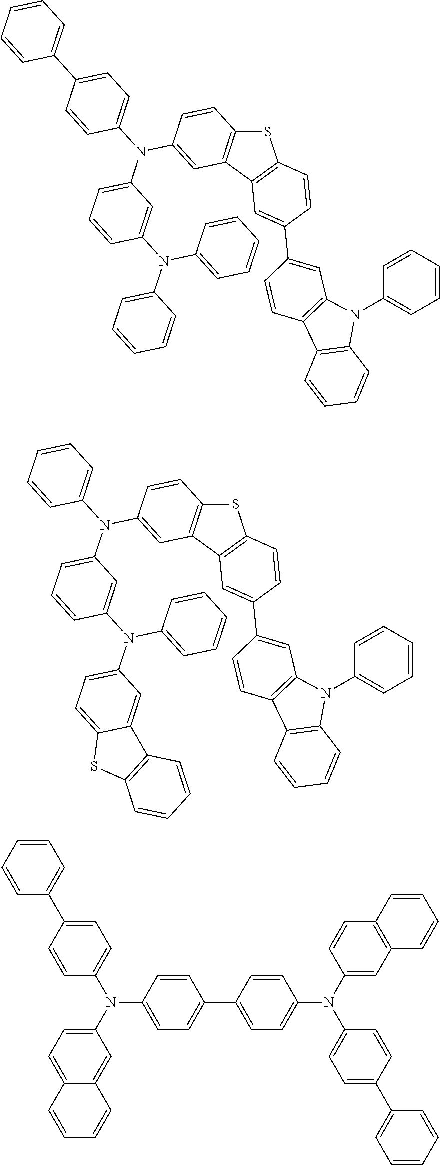 [SMILES: c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1]

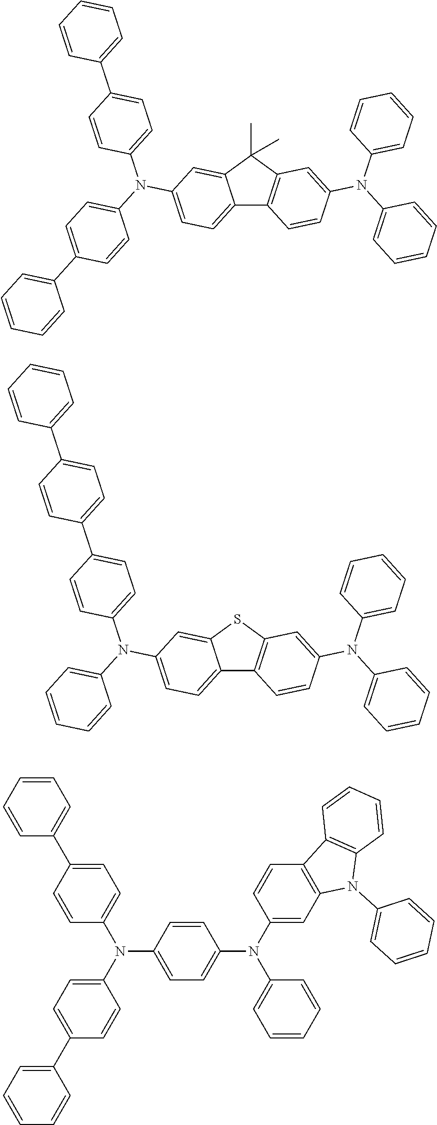

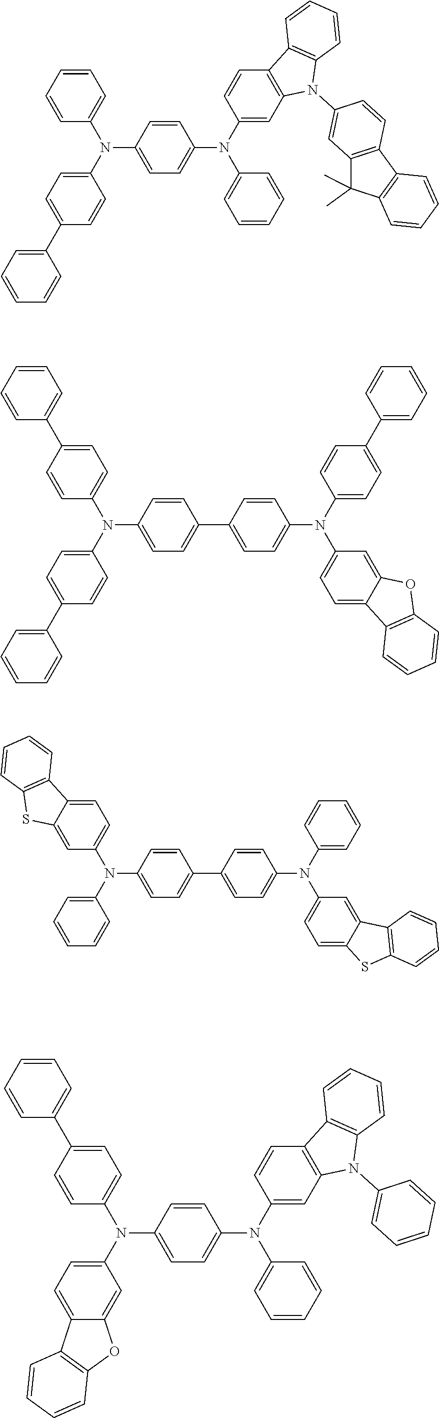 [SMILES: CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21 ; c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1 ; c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1]

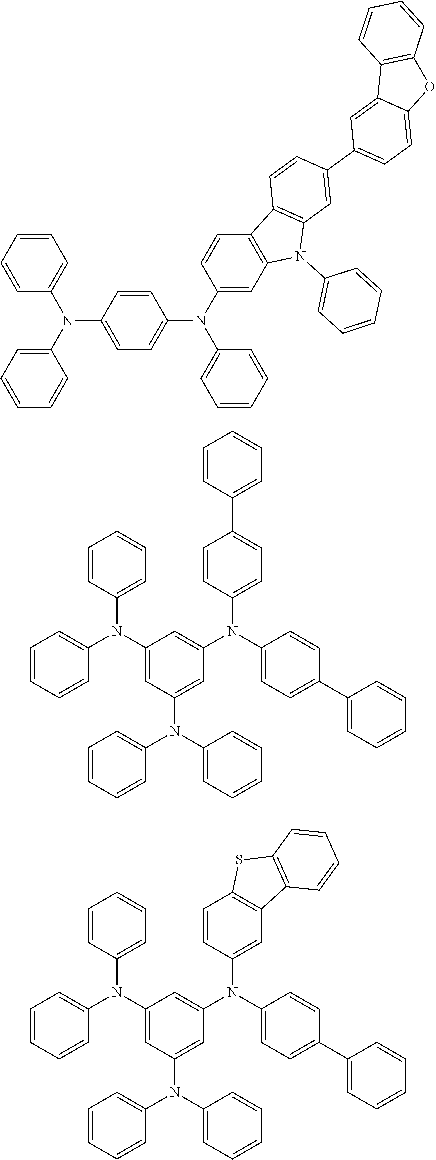

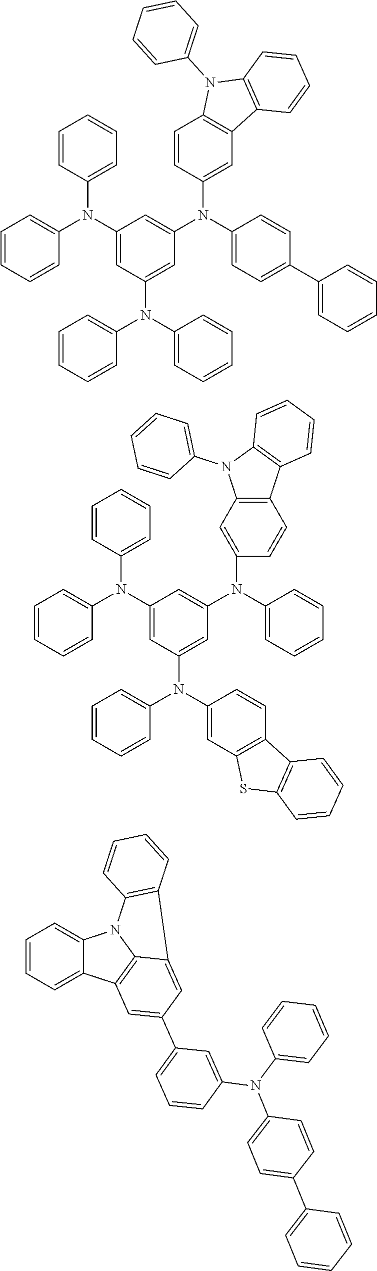

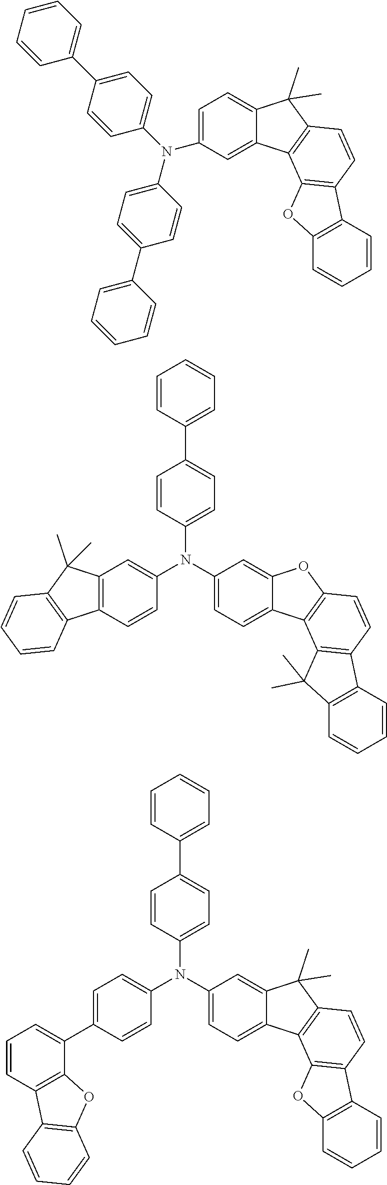

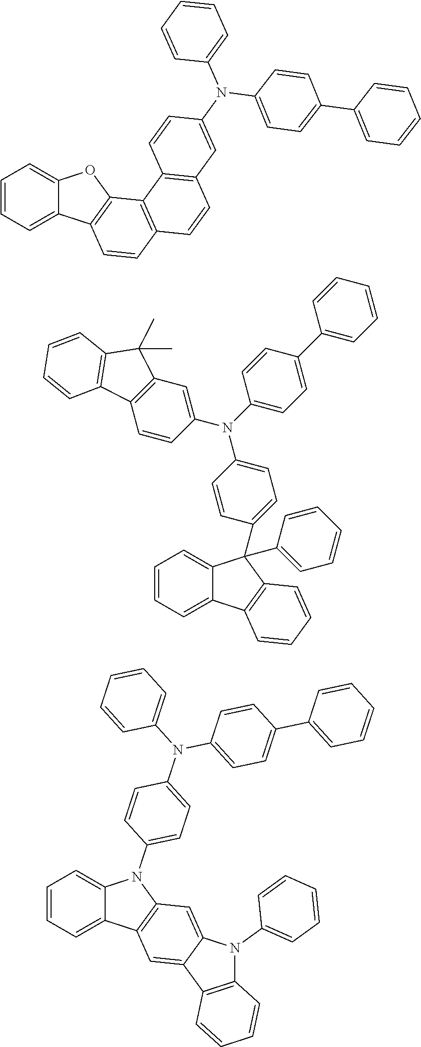 [SMILES: c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1]

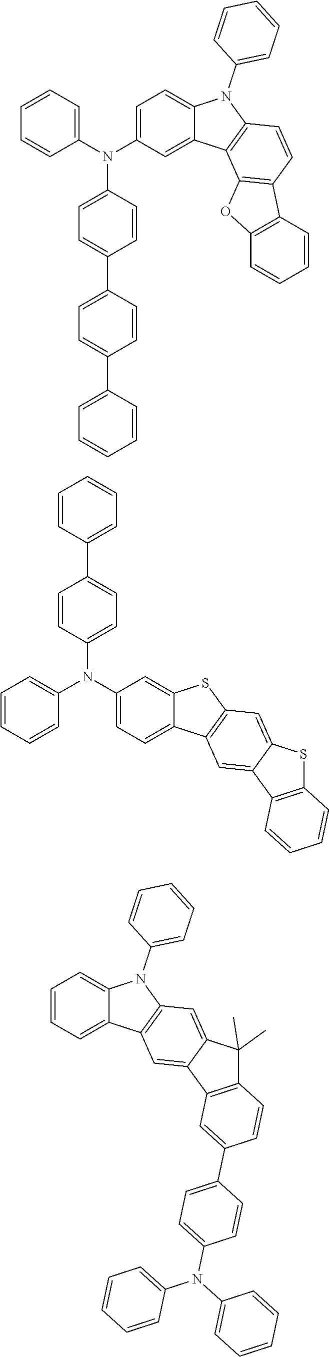

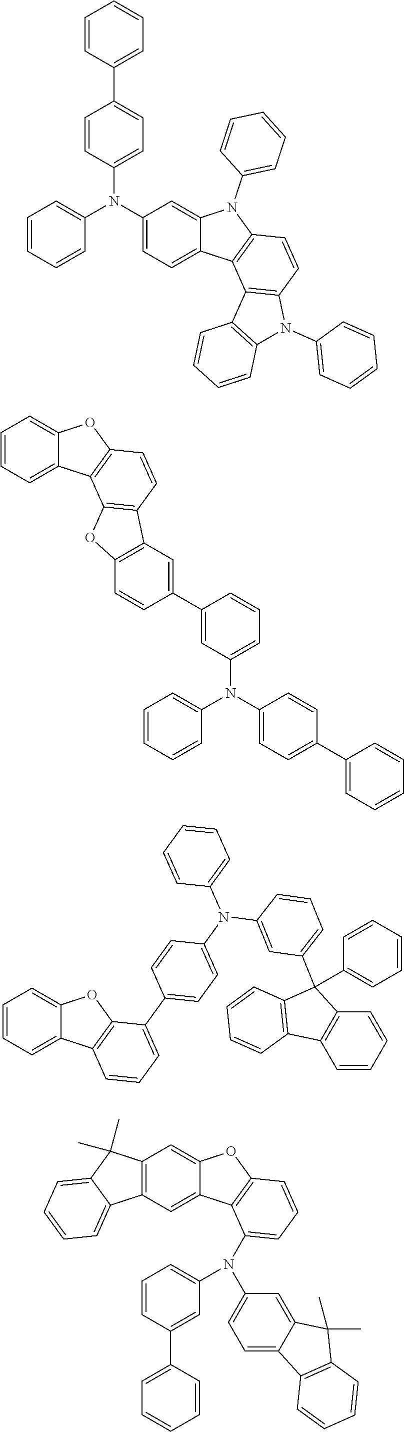

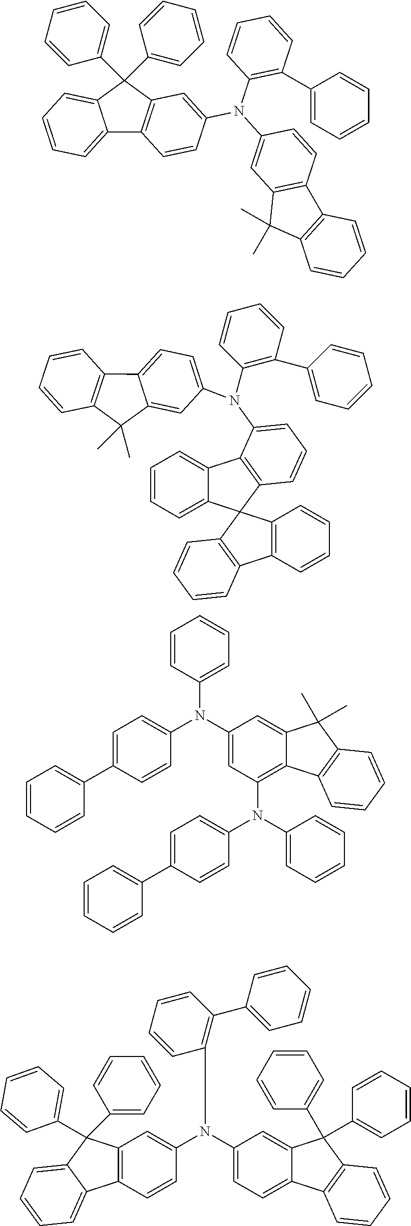

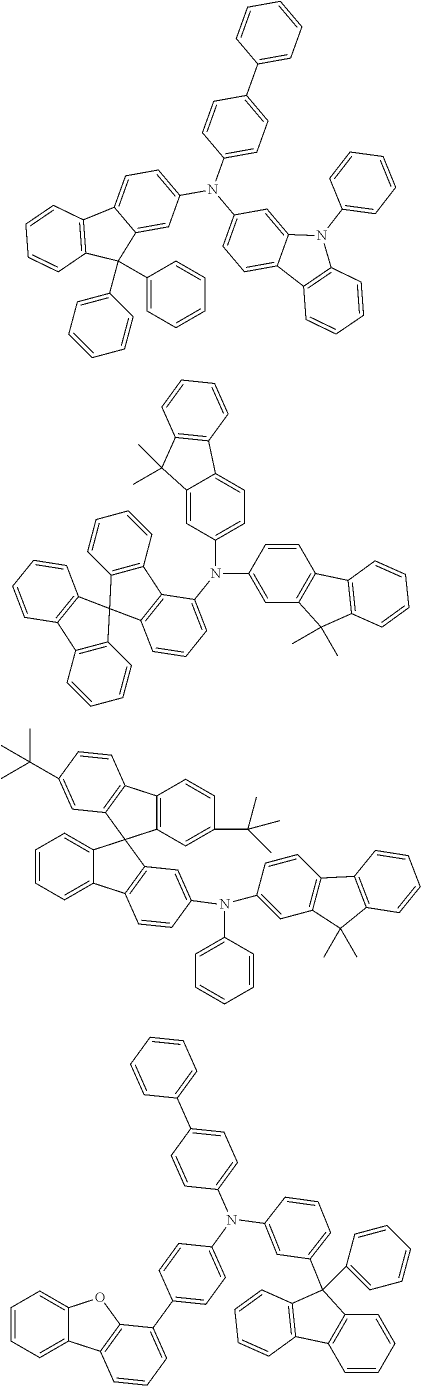 [SMILES: c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2 ; c1ccc(-c2ccc(N(c3ccc(-c4cccc5c4oc4ccccc45)cc3)c3cccc(C4(c5ccccc5)c5ccccc5-c5ccccc54)c3)cc2)cc1]

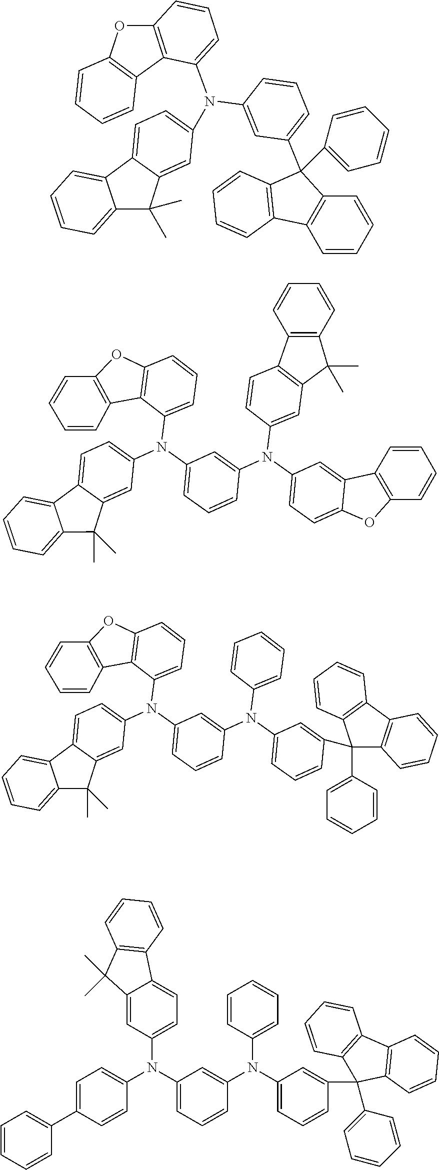

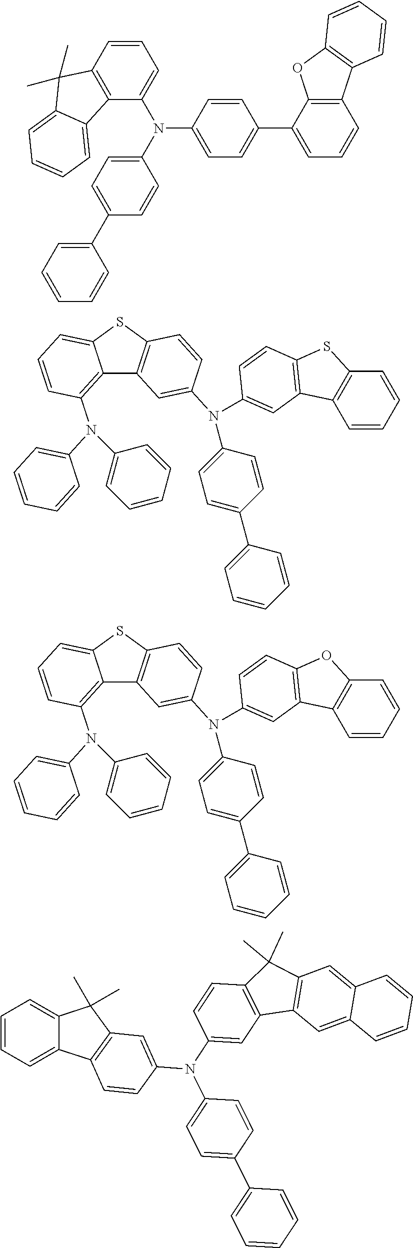

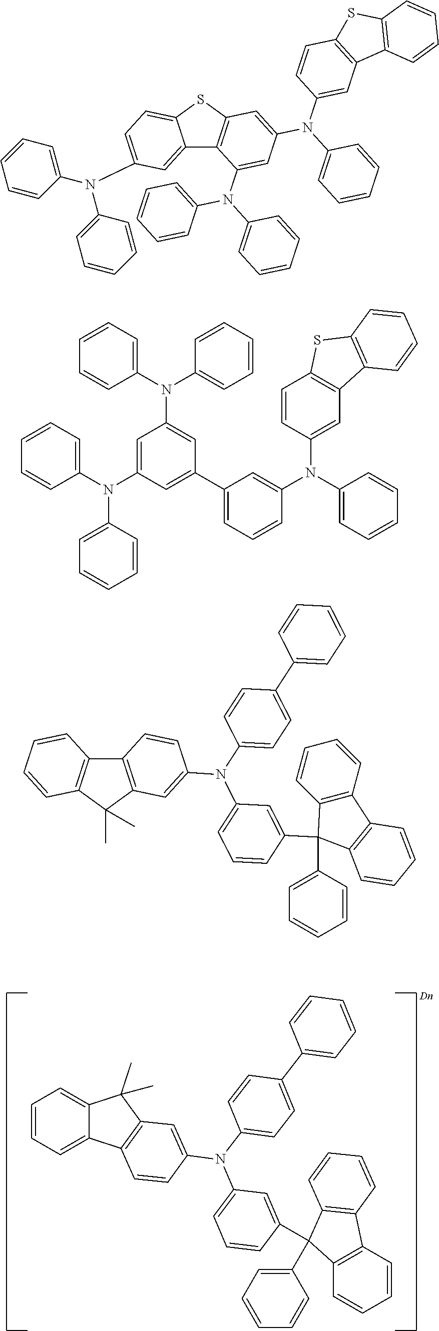

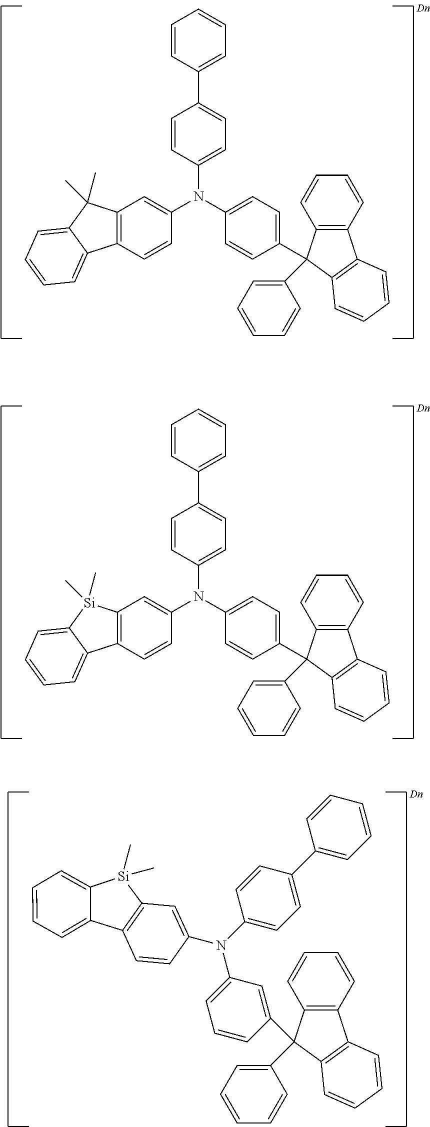

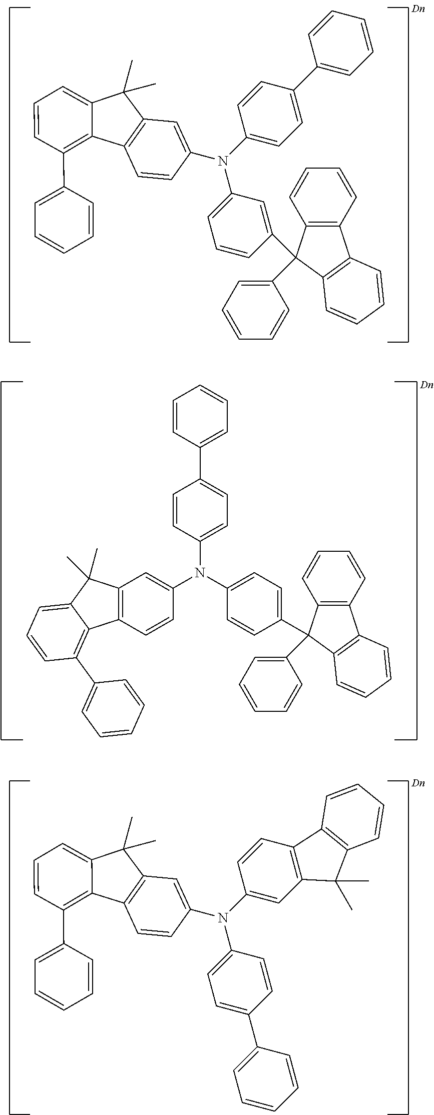

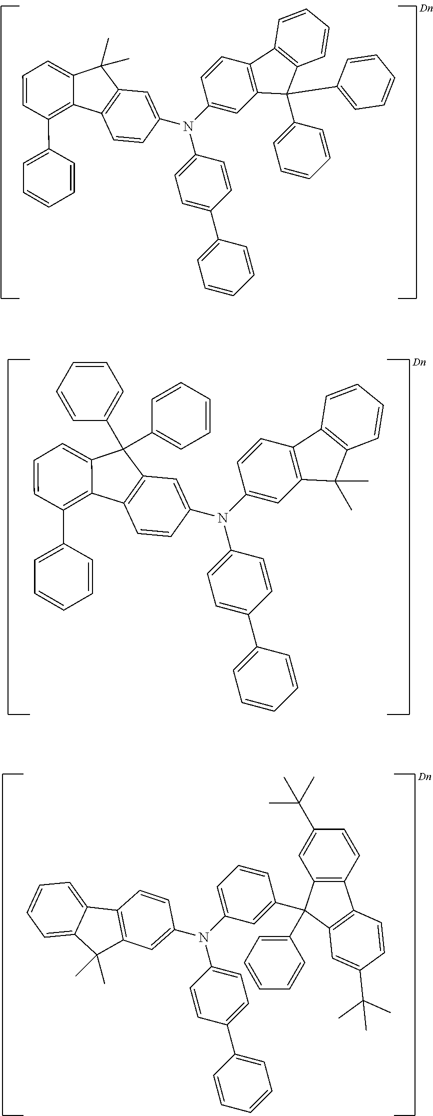

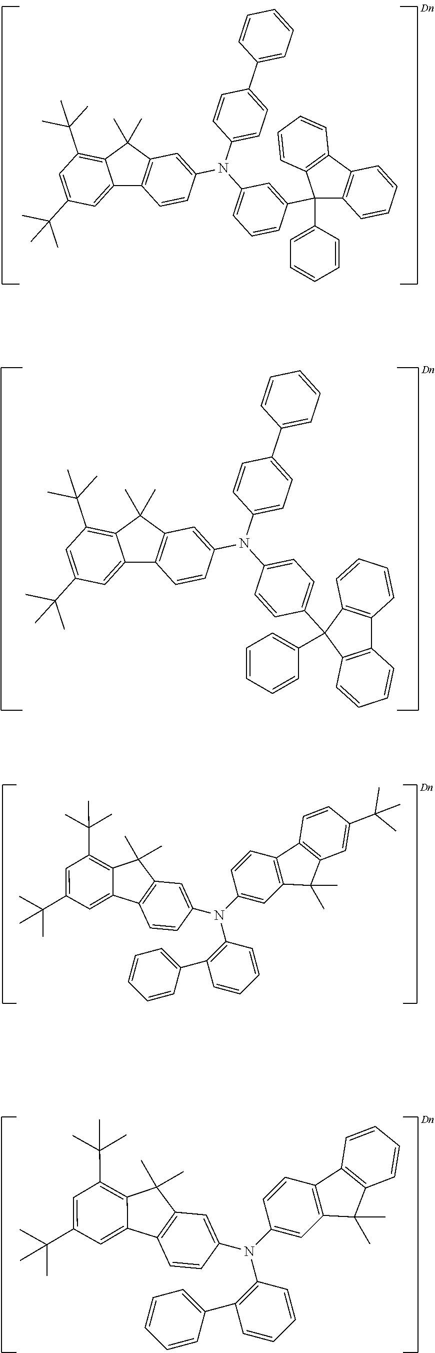

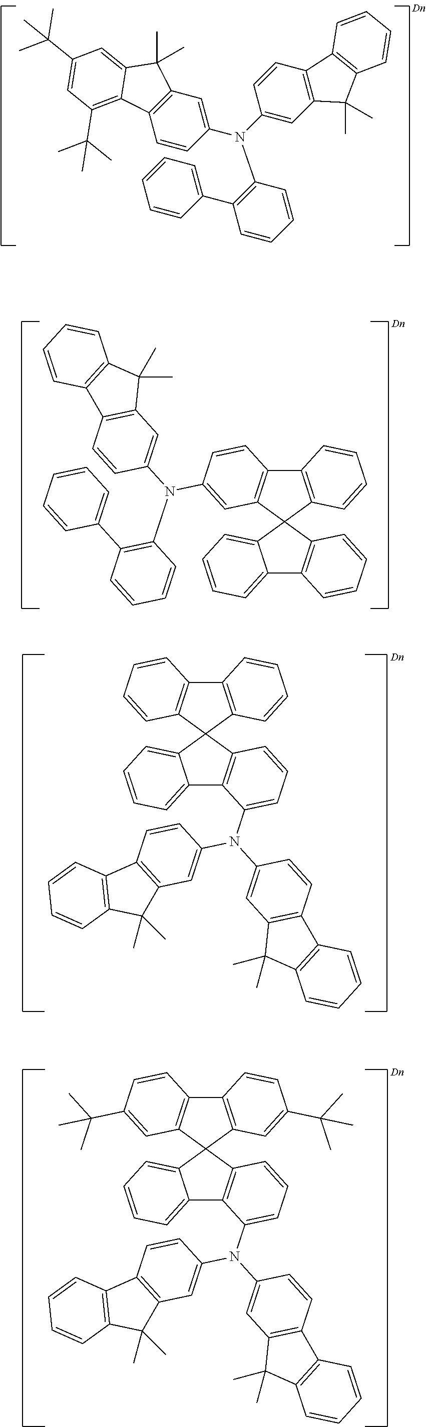

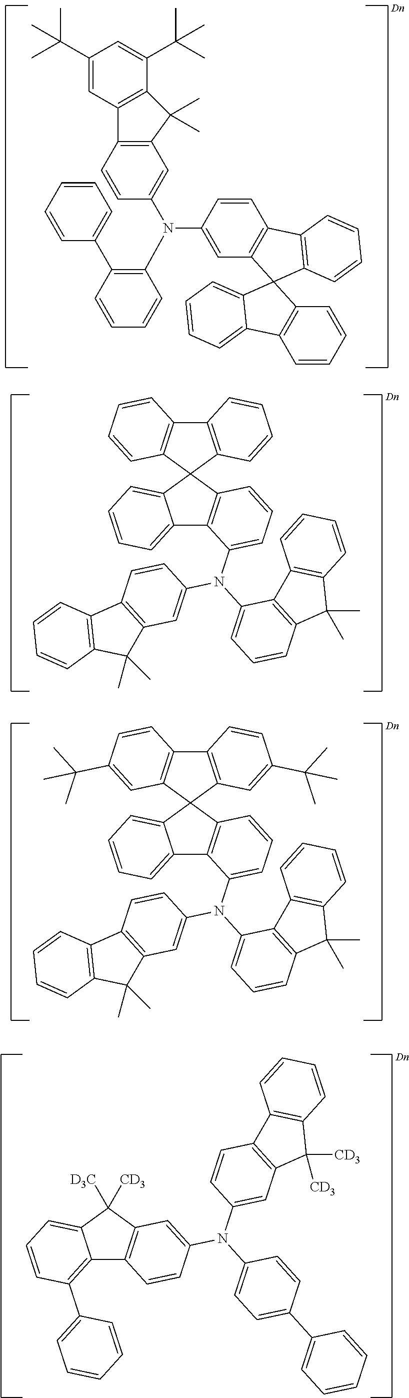

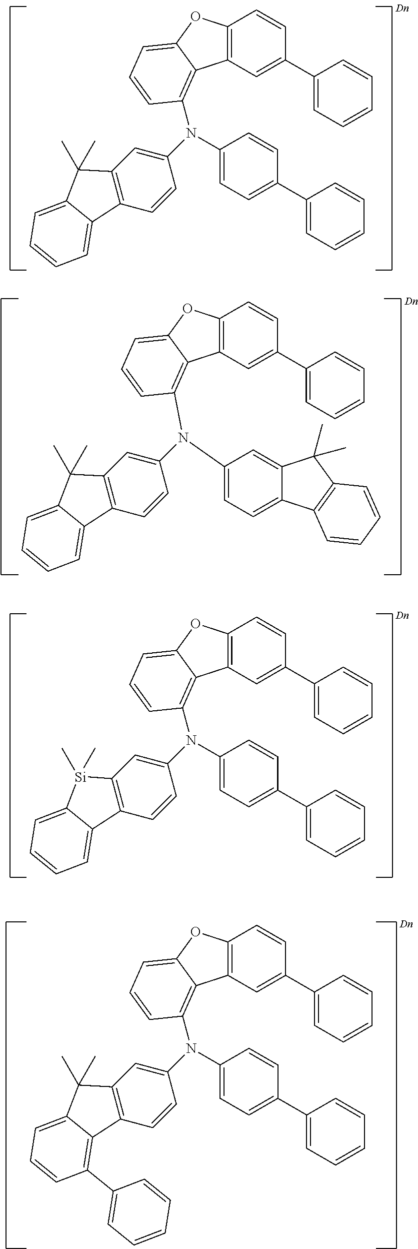

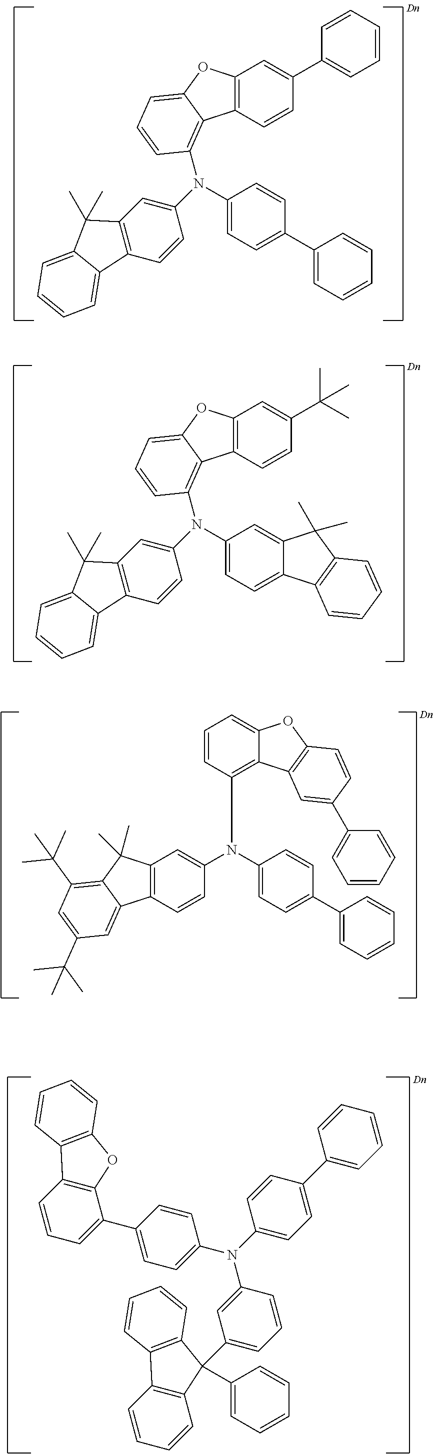

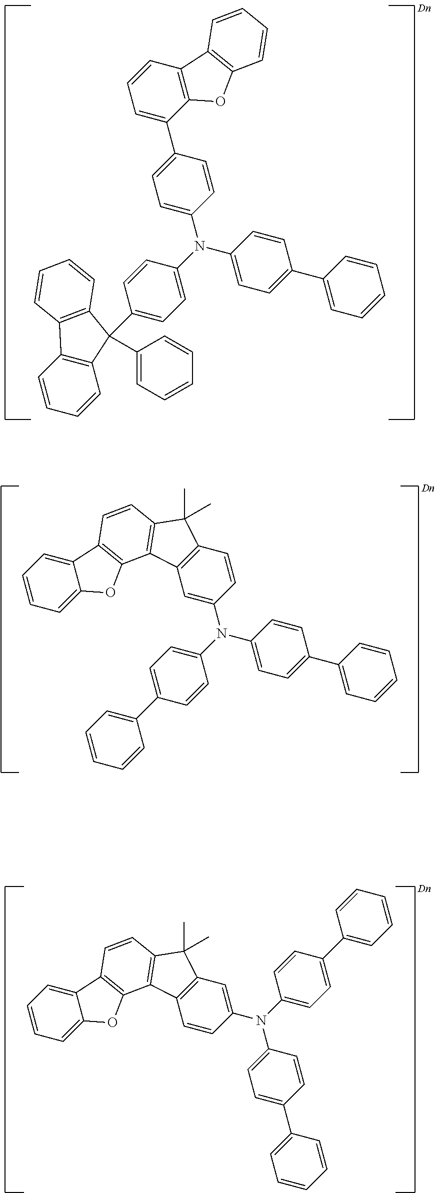

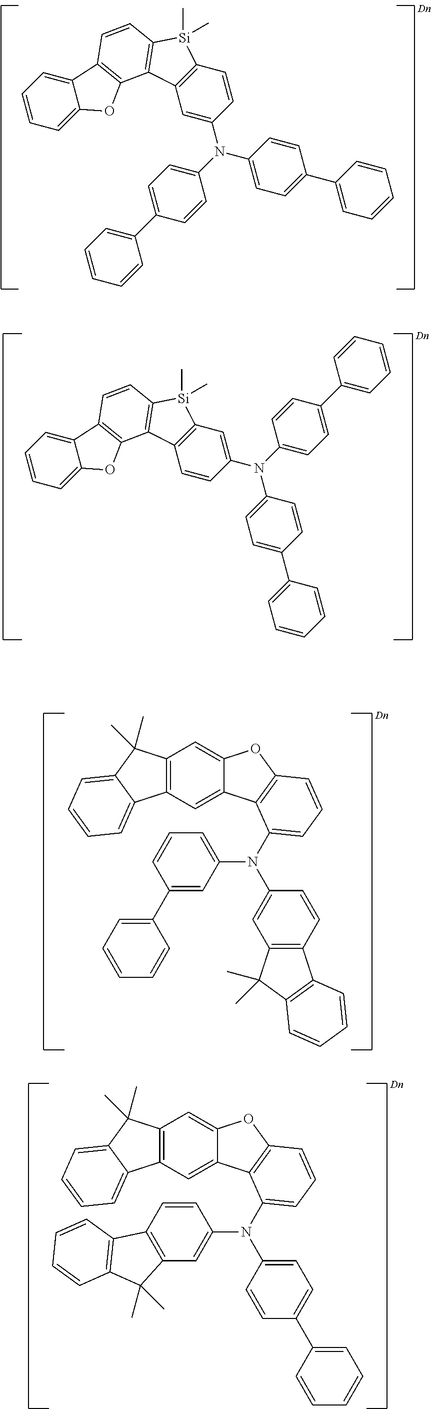

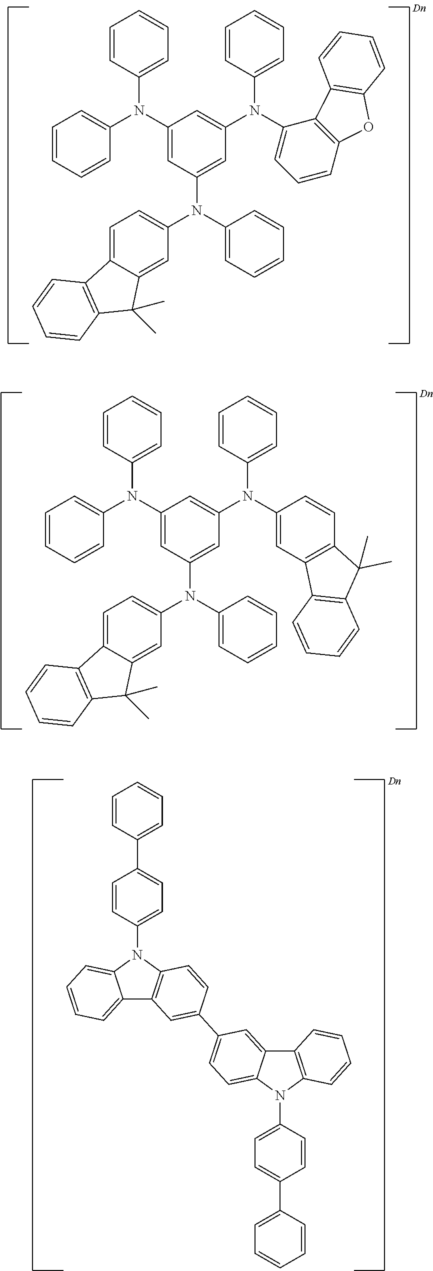

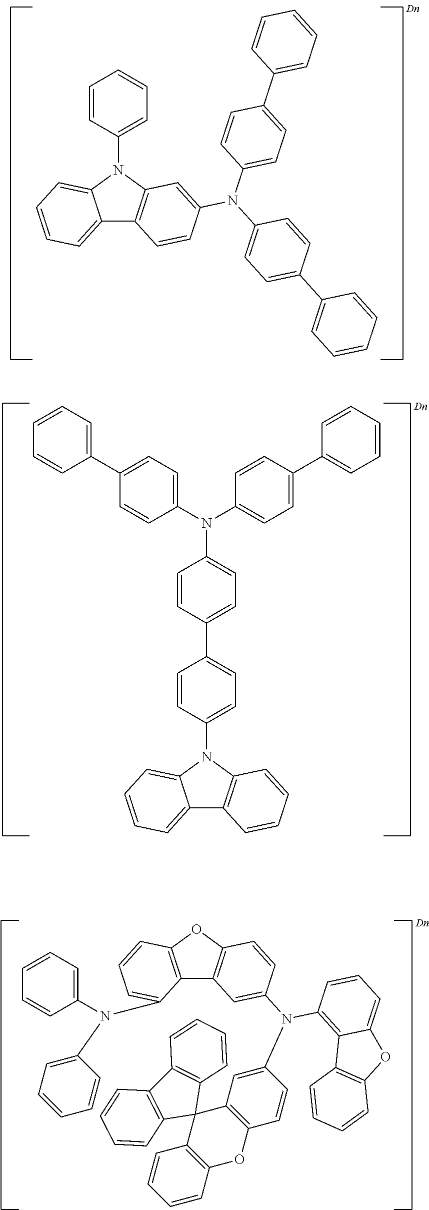

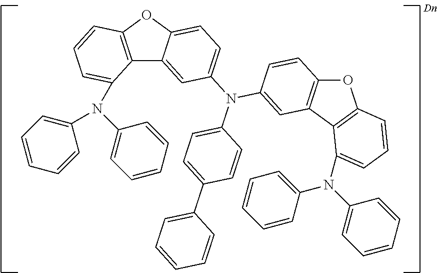

(Dn refers to the number of deuterium atoms substituted, and indicates a structure in which one or more deuterium atoms are substituted)

In the hole transport region 140, in addition to the compounds described above, known compounds disclosed in U.S. Pat. No. 5,061,569A, JP1993-009471A, WO1995-009147A1, JP1995-126615A, JP1998-095973A, etc. and compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

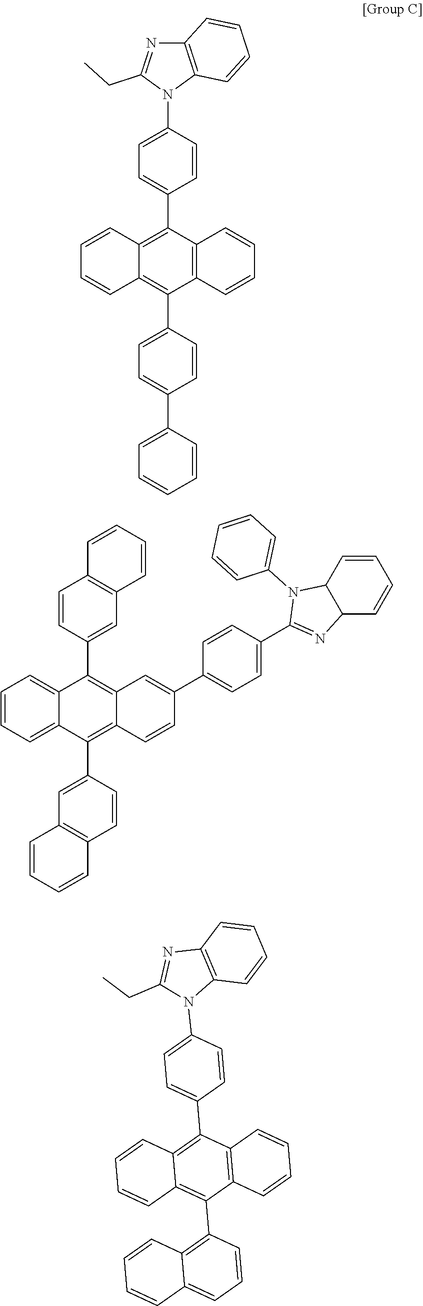 [SMILES: CCc1nc2ccccc2n1-c1ccc(-c2c3ccccc3c(-c3ccc(-c4ccccc4)cc3)c3ccccc23)cc1 ; C1=CC2N=C(c3ccc(-c4ccc5c(-c6ccc7ccccc7c6)c6ccccc6c(-c6ccc7ccccc7c6)c5c4)cc3)N(c3ccccc3)C2C=C1 ; CCc1nc2ccccc2n1-c1ccc(-c2c3ccccc3c(-c3cccc4ccccc34)c3ccccc23)cc1]

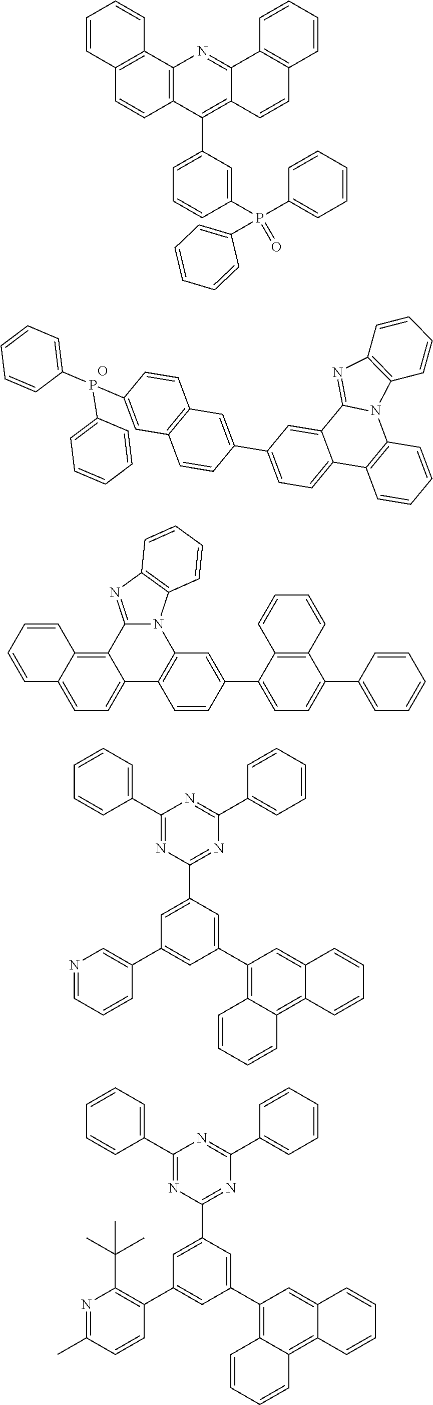

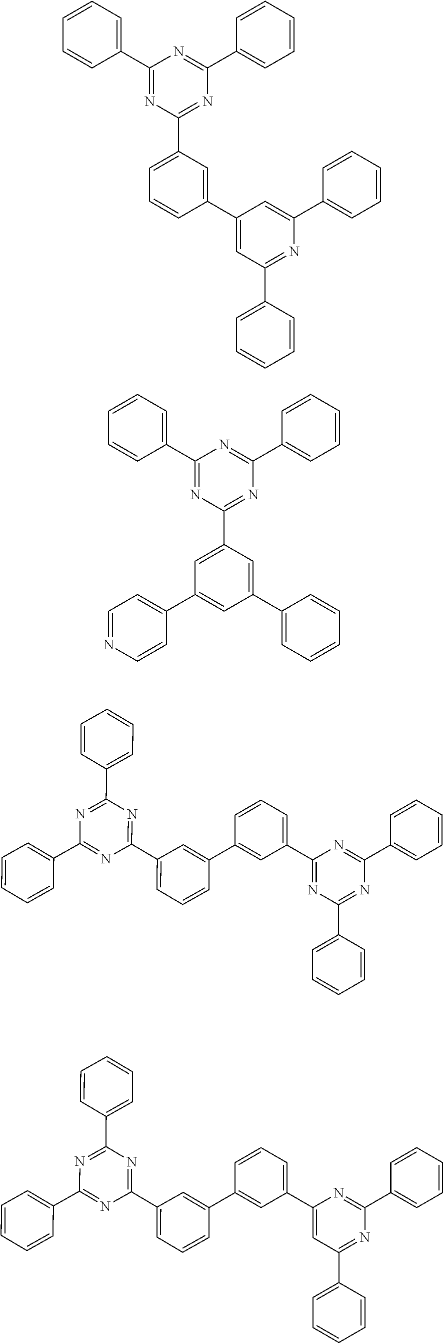 [SMILES: c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccncc3)cc(-c3nc(-c4ccccc4)nc(-c4ccccc4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

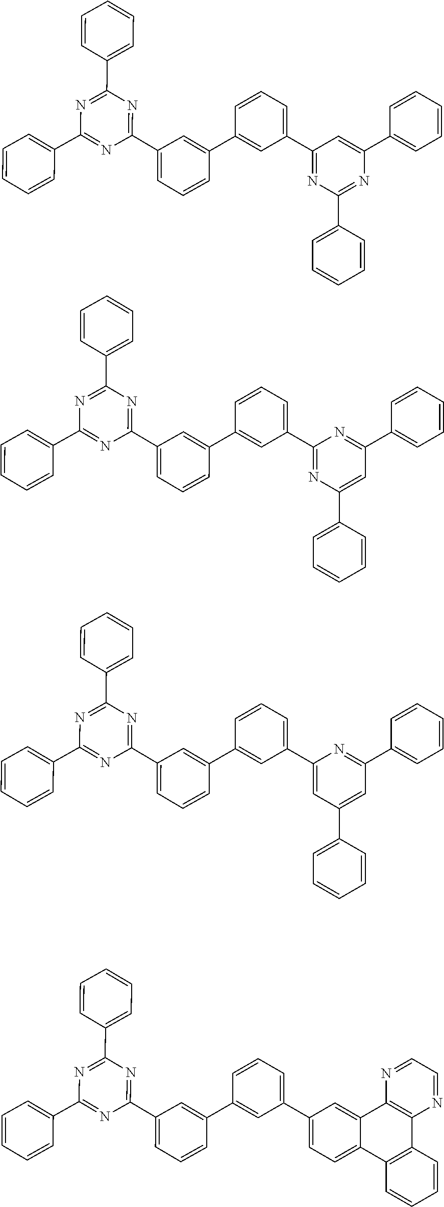 [SMILES: c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1]

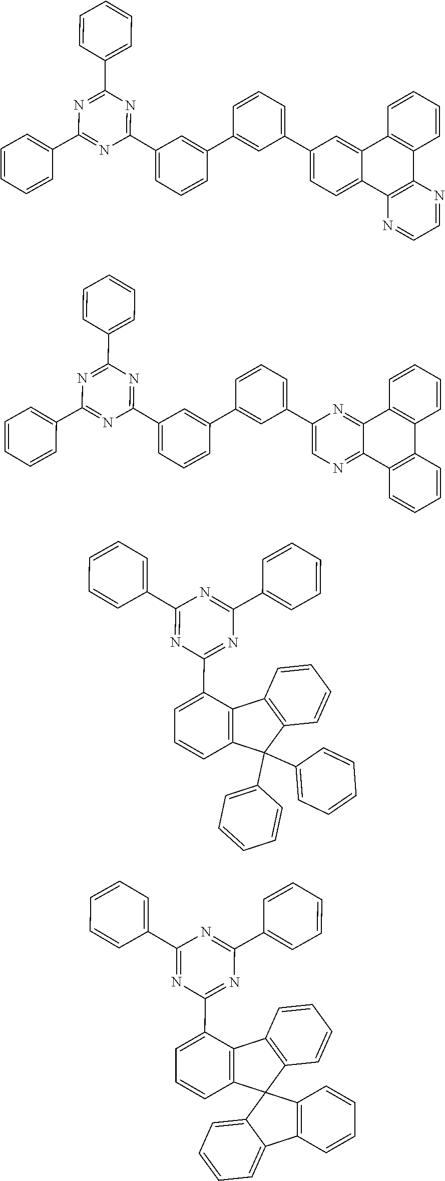 [SMILES: c1ccc(-c2nc(-c3ccccc3)nc(-c3cccc(-c4cccc(-c5ccc6c(c5)c5ccccc5c5nccnc65)c4)c3)n2)cc1 ; c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1 ; c1ccc(-c2nc(-c3ccccc3)nc(-c3cccc4c3-c3ccccc3C43c4ccccc4-c4ccccc43)n2)cc1]

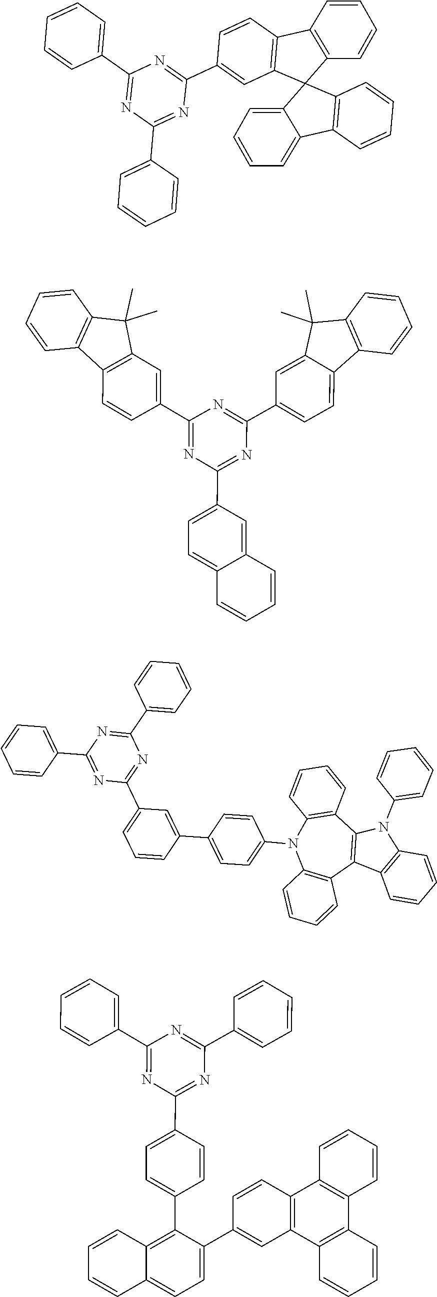 [SMILES: c1ccc(-c2nc(-c3ccccc3)nc(-c3ccc4c(c3)C3(c5ccccc5-c5ccccc53)c3ccccc3-4)n2)cc1 ; CC1(C)c2ccccc2-c2ccc(-c3nc(-c4ccc5c(c4)C(C)(C)c4ccccc4-5)nc(-c4ccc5ccccc5c4)n3)cc21 ; c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1 ; c1ccc(-c2nc(-c3ccccc3)nc(-c3ccc(-c4c(-c5ccc6c7ccccc7c7ccccc7c6c5)ccc5ccccc45)cc3)n2)cc1]

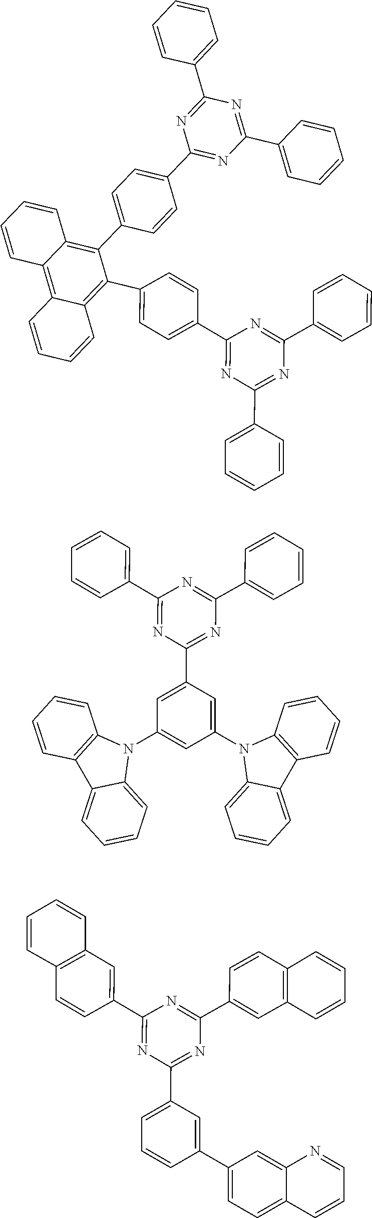 [SMILES: c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2ccc3cccnc3c2)cc(-c2nc(-c3ccc4ccccc4c3)nc(-c3ccc4ccccc4c3)n2)c1]

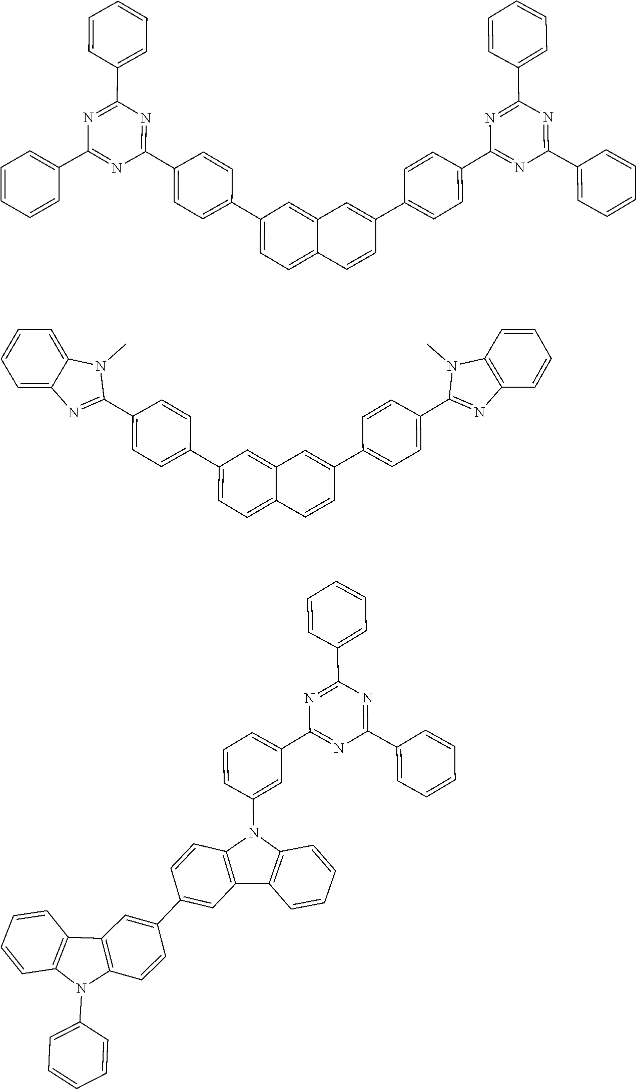 [SMILES: c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1 ; Cn1c(-c2ccc(-c3ccc4ccc(-c5ccc(-c6nc7ccccc7n6C)cc5)cc4c3)cc2)nc2ccccc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

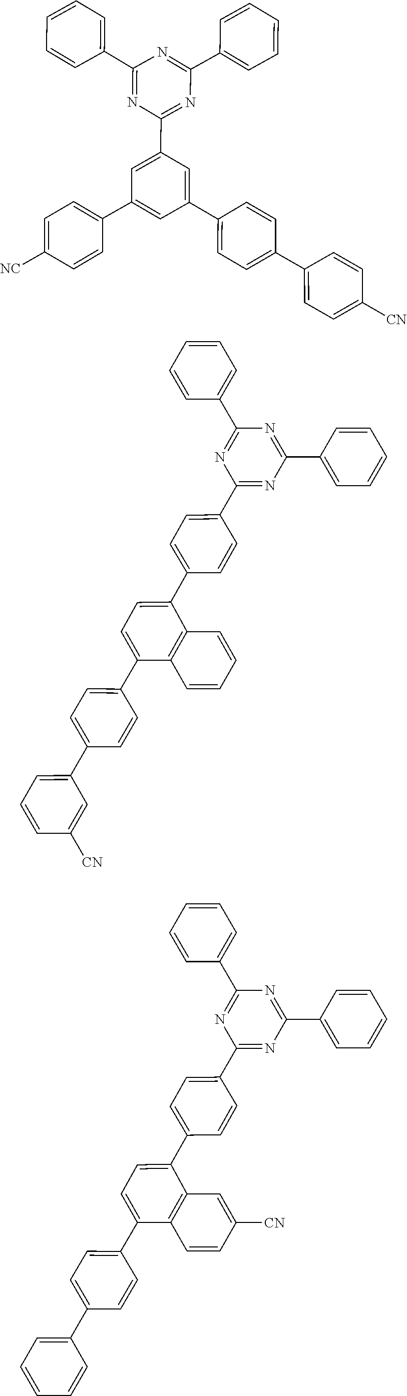 [SMILES: N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

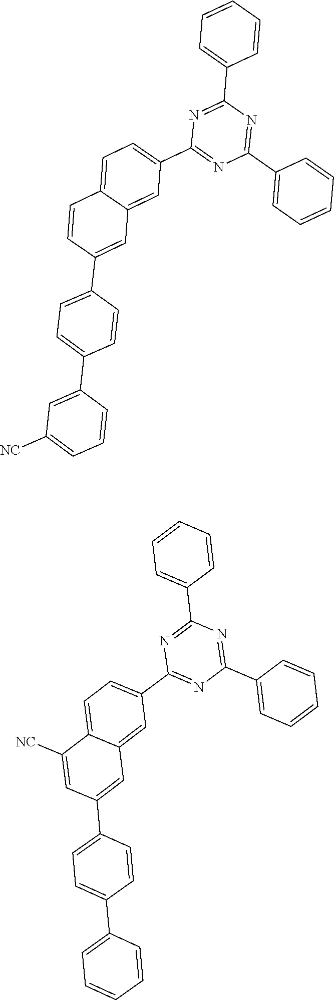 [SMILES: N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12]

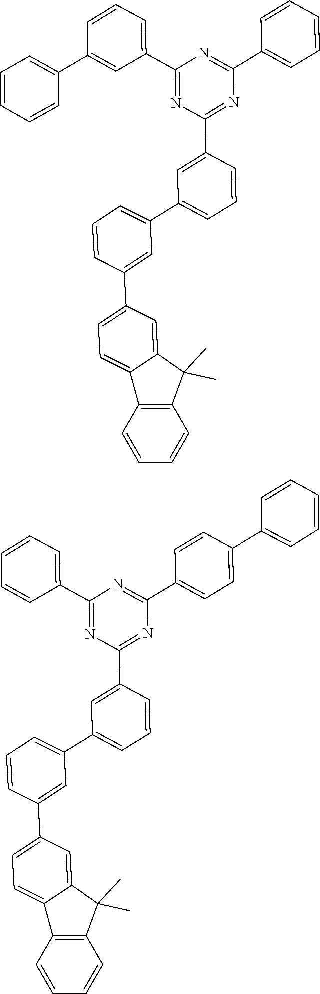 [SMILES: CC1(C)c2ccccc2-c2ccc(-c3cccc(-c4cccc(-c5nc(-c6ccccc6)nc(-c6cccc(-c7ccccc7)c6)n5)c4)c3)cc21 ; CC1(C)c2ccccc2-c2ccc(-c3cccc(-c4cccc(-c5nc(-c6ccccc6)nc(-c6ccc(-c7ccccc7)cc6)n5)c4)c3)cc21]

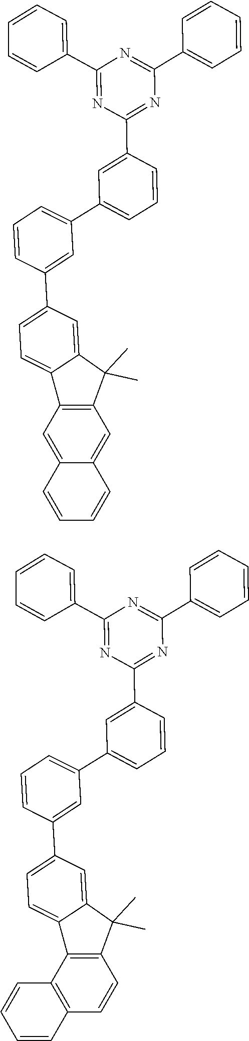

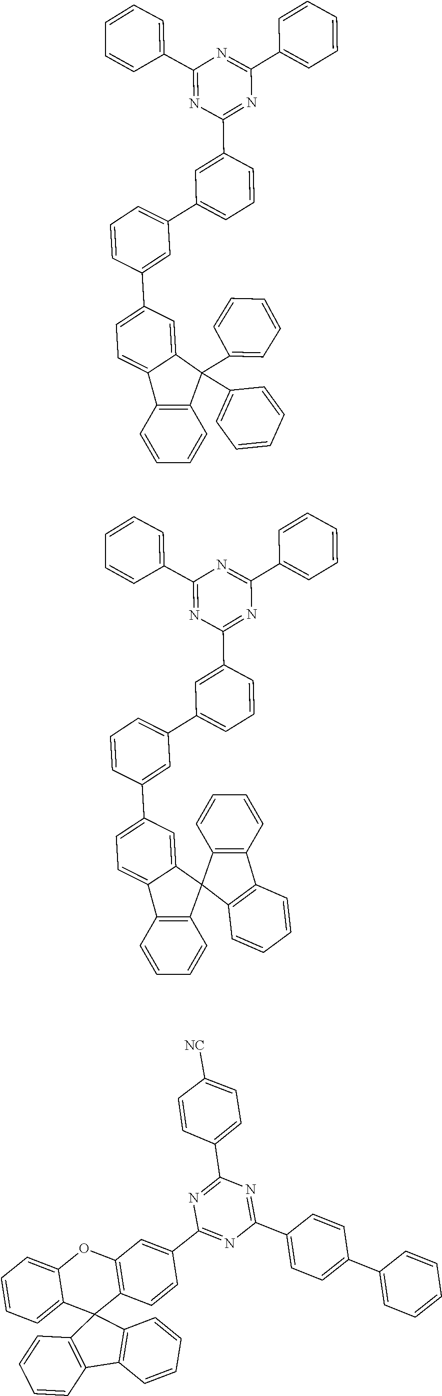 [SMILES: c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1 ; c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1 ; N#Cc1ccc(-c2nc(-c3ccc(-c4ccccc4)cc3)nc(-c3ccc4c(c3)Oc3ccccc3C43c4ccccc4-c4ccccc43)n2)cc1]

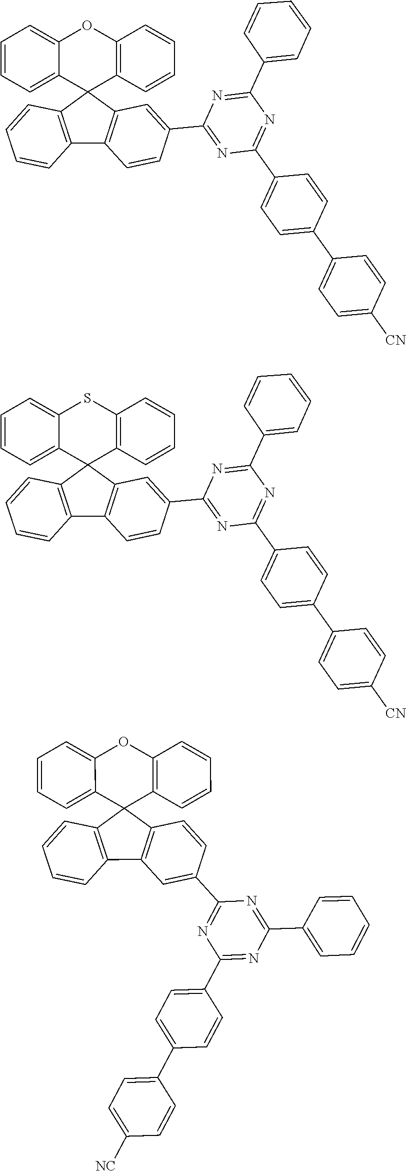 [SMILES: N#Cc1ccc(-c2ccc(-c3nc(-c4ccccc4)nc(-c4ccc5c(c4)C4(c6ccccc6Oc6ccccc64)c4ccccc4-5)n3)cc2)cc1 ; N#Cc1ccc(-c2ccc(-c3nc(-c4ccccc4)nc(-c4ccc5c(c4)C4(c6ccccc6Sc6ccccc64)c4ccccc4-5)n3)cc2)cc1 ; N#Cc1ccc(-c2ccc(-c3nc(-c4ccccc4)nc(-c4ccc5c(c4)-c4ccccc4C54c5ccccc5Oc5ccccc54)n3)cc2)cc1]

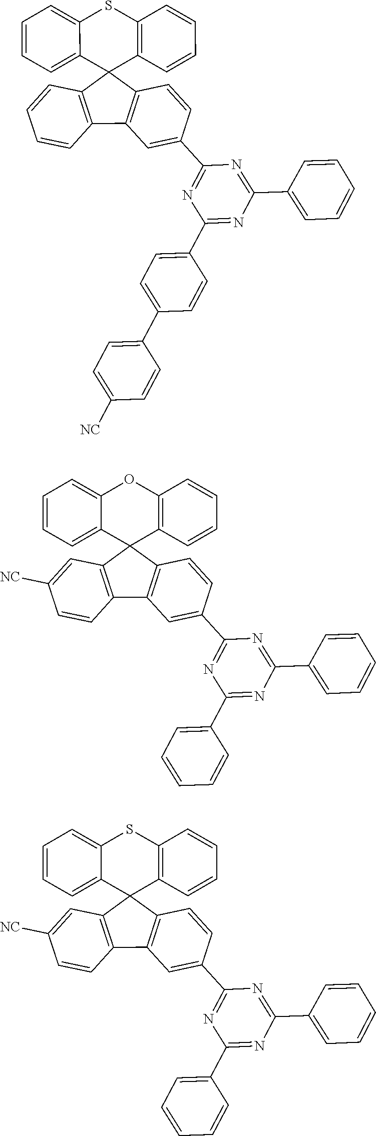

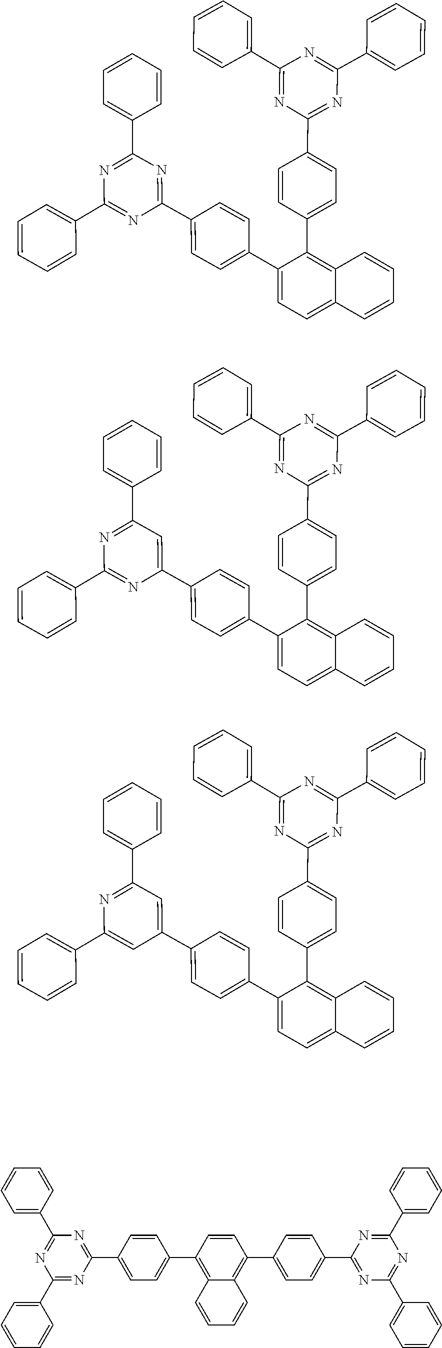 [SMILES: c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1 ; c1ccc(-c2cc(-c3ccc(-c4ccc5ccccc5c4-c4ccc(-c5nc(-c6ccccc6)nc(-c6ccccc6)n5)cc4)cc3)nc(-c3ccccc3)n2)cc1 ; c1ccc(-c2cc(-c3ccc(-c4ccc5ccccc5c4-c4ccc(-c5nc(-c6ccccc6)nc(-c6ccccc6)n5)cc4)cc3)cc(-c3ccccc3)n2)cc1 ; c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1]

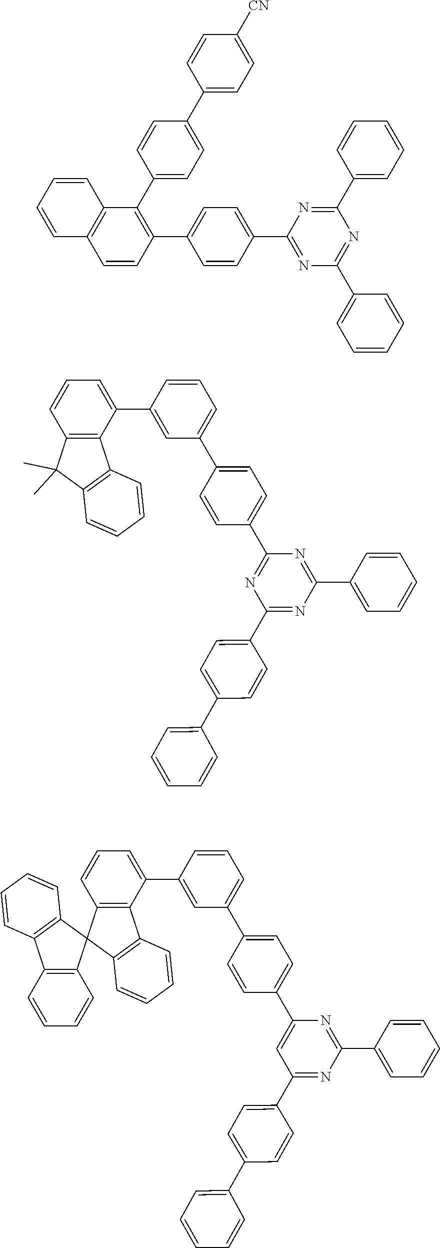 [SMILES: N#Cc1ccc(-c2ccc(-c3c(-c4ccc(-c5nc(-c6ccccc6)nc(-c6ccccc6)n5)cc4)ccc4ccccc34)cc2)cc1 ; CC1(C)c2ccccc2-c2c(-c3cccc(-c4ccc(-c5nc(-c6ccccc6)nc(-c6ccc(-c7ccccc7)cc6)n5)cc4)c3)cccc21 ; c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1]

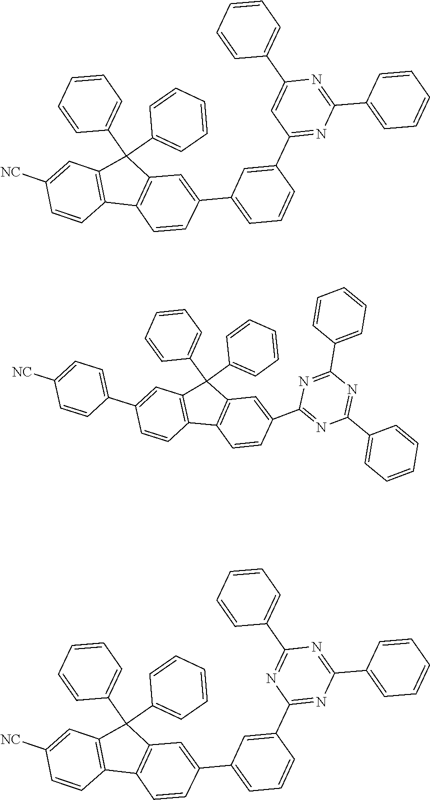 [SMILES: N#Cc1ccc2c(c1)C(c1ccccc1)(c1ccccc1)c1cc(-c3cccc(-c4cc(-c5ccccc5)nc(-c5ccccc5)n4)c3)ccc1-2 ; N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

An embodiment may be an organic light emitting diode including the light emitting layer as the organic layer.

Another embodiment may be an organic light emitting diode including a light emitting layer and a hole transport region as the organic layer.

Another embodiment may be an organic light emitting diode including a light emitting layer and an electron transport region as the organic layer.

An organic light emitting diode according to an embodiment includes a hole transport region 140 and an electron transport region 150 in addition to the light emitting layer 130 as the organic layer 105, as shown in FIG. 1.

On the other hand, an organic light emitting diode may further include an electron injection layer (not shown), a hole injection layer (not shown), etc. in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

Hereinafter, the embodiments are illustrated in more detail with reference to examples. However, these examples are exemplary, and the present scope is not limited thereto.

### MODE FOR INVENTION

Hereinafter, starting materials and reactants used in Examples and Synthesis Examples were purchased from Sigma-Aldrich Co. Ltd., TCI Inc., Tokyo chemical industry, or P&H tech Co., Ltd. as far as there is no particular comment or were synthesized by known methods.

### Preparation of Compound for Organic Optoelectronic Device

### Synthesis of First Compound

### Synthesis Example 1: Synthesis of Compound 10

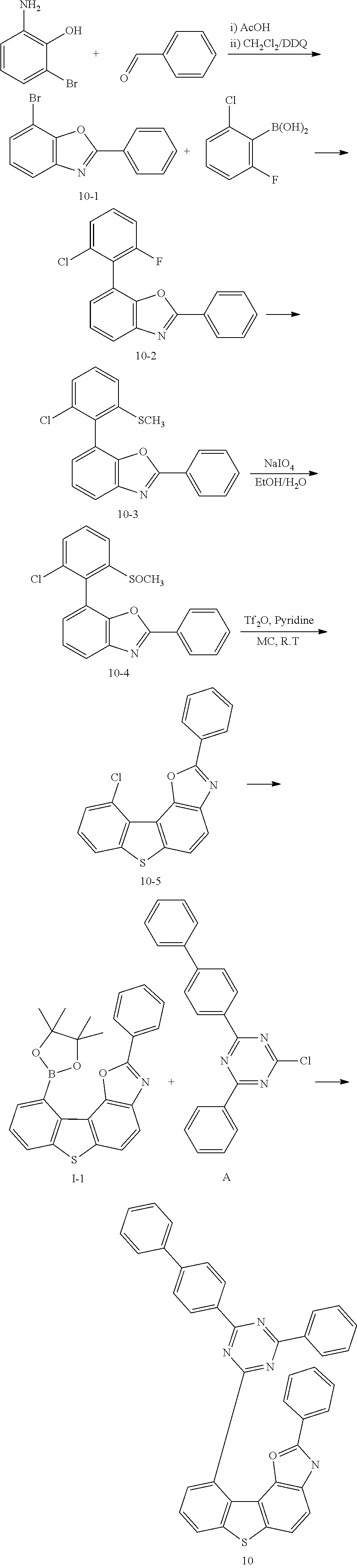

### 1 st Step: Synthesis of Intermediate 10-1

2-amino-6-bromophenol (100.0 g, 485.83 mmol, Merck & Co., Inc.) and benzaldehyde (43.0 g, 404.86 mmol, Merck & Co., Inc.) were dissolved in acetic acid (120 g, 2024.29 mmol) and then, reacted at room temperature for 3 hours. Subsequently, the reactant was poured into a large amount of DIW and then, stirred for 30 minutes, filtered, and twice washed with water. A solid was separated therefrom, extracted with dichloromethane and DIW, and dried with magnesium sulfate, and all the solvents were removed. Subsequently, the solid was dissolved in dichloromethane in an amount corresponding to 10 times that of the solid, and 84.9 g (374 mmol) of 2,3-dichloro-5,6-dicyano-p-benzoquinone (DDQ) was slowly added thereto and then, reacted at room temperature for 2 hours, obtaining benzoxazole of Intermediate 10-1 (70 g, Yield: 78%).

### 2nd Step: Synthesis of Intermediate 10-2

Intermediate 10-1 (70.0 g, 255.36 mmol) and (2-chloro-6-fluorophenyl) boronic acid (53.43 g, 306.44 mmol), Pd(PPh₃)₄ (13.85 g, 11.98 mmol), and K₂CO₃ (99.36 g, 718.91 mmol) were added to dioxane (600 ml) and DIW (3000 ml) and dissolved therein and then, refluxed by heating under a nitrogen atmosphere. After 12 hours, the reaction solution was cooled, and after removing an aqueous layer and also, removing the solvents with a rotary evaporator, extraction was performed with methylene chloride/DIW. An organic layer therefrom was columned with hexane:EA=4:1 (v/v), obtaining Intermediate 10-2 (60.0 g, Yield: 69%).

### 3rd Step: Synthesis of Intermediate 10-3

Intermediate 10-2 (60.0 g, 185.33 mmol) and K₂CO₃ (38.42 g, 277.99 mmol) were dissolved in dimethyl formamide (600 mL), and after setting a temperature of the reaction solution at 0° C., 15.0 g (203.86 mmol) of sodium thiomethoxide was slowly added thereto. When the addition was completed, after increasing the temperature to 100° C., the reaction solution was heated under reflux for 2 hours. A solid was filtered therefrom, extracted with ethylacetate and DIW, and after volatilizing a solvent layer, recrystallized with hexane, obtaining Intermediate 10-3 (38.0 g, Yield: 58%).

### 4th Step: Synthesis of Intermediate 10-4

Intermediate 10-3 (38.0 g, 108.0 mmol) and sodium periodate (34.65 g, 162.0 mmol) were dissolved in 700 mL of a mixed solvent of ethanol/DIW and then, stirred under reflux at 50° C. for 12 hours. When a reaction was completed, an 1 M sodium thiosulfate aqueous solution was added to the reaction solution and then, stirred for 1 hour and extracted with an excessive amount of methylene chloride, and the solvent was volatilized therefrom, obtaining Intermediate 10-4 (35.0 g, Yield: 88%).

### 5th Step: Synthesis of Intermediate 10-5

Intermediate 10-4 (35.0 g, 95.15 mmol) was dissolved in 500 mL of 1,2-dichloroethane, and after setting a temperature of the reaction solution at 0° C., 53.69 g (190.29 mmol) of triflic anhydride was slowly added thereto in a dropwise fashion, and after increasing the reaction temperature to room temperature, the mixture was stirred for 1 hour. After setting the internal temperature at 5° C., pyridine (22.57 g, 142.72 mmol) was slowly added thereto in a dropwise fashion and reacted at room temperature for 12 hours. The obtained solid was filtered and washed with ethanol, and the solvent was removed therefrom, obtaining Intermediate 10-5 (22.0 g, Yield: 69%).

### 6th Step: Synthesis of Intermediate I-1

Intermediate 10-5 (22.0 g, 65.51 mmol), bis(pinacolato)diboron (19.96 g, 78.62 mmol), Pd₂(dba)₃ (3.60 g, 3.93 mmol), P(Cy)₃ (4.41 g, 15.72 mmol), and KOAc (19.29 g, 196.54 mmol) were dissolved in 300 mL of xylene and then, stirred under reflux at 150° C. for 12 hours. When a reaction was completed, after removing the reaction solvent with a rotary evaporator, an organic layer, which was extracted with methylene chloride, was columned with hexane:EA=4:1 (v/v), obtaining Intermediate I-1 (18.0 g, Yield: 64%).

### 7 Step: Synthesis of Compound 10

In a round-bottomed flask, Intermediate I-1 (18 g, 41.59 mmol), 2-([1,1′-biphenyl]-4-yl)-4-chloro-6-phenyl-1,3,5-triazine purchased from P&H Tech Co., Ltd. (http://www.phtech.co.kr/) (Intermediate A, 13.0 g, 37.81 mmol), Pd(PPh₃)₄ (2.18 g, 1.89 mmol), and K₂CO₃ (13.06 g, 94.53 mmol) were dissolved in THF (100 mL) and distilled water (50 mL) and then, refluxed by heating under a nitrogen atmosphere. After 12 hours, when the reaction solution was cooled, after removing an aqueous layer, an organic layer therefrom was dried under a reduced pressure. The obtained solid was washed with water and methanol and twice recrystallized with 200 mL of toluene, obtaining Compound 10 (18.2 g, Yield: 70%).

LC/MS calculated for: C40H24N4OS Exact Mass 608.17 found for 609.33 [M+H].

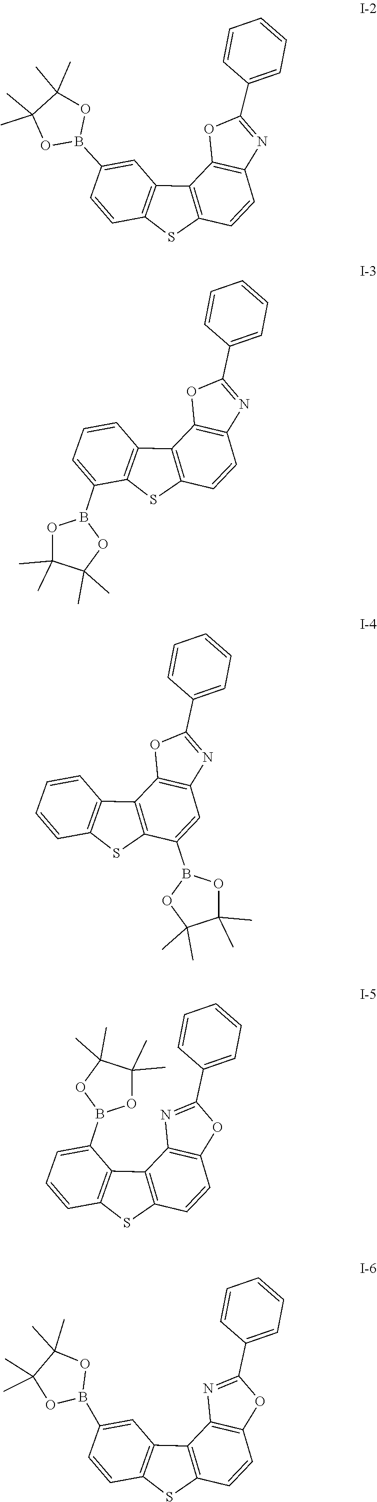

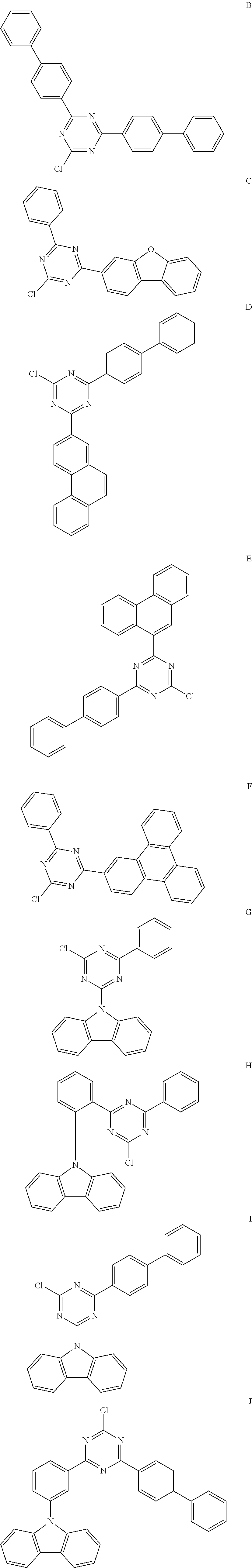

### Synthesis Example 2: Synthesis of Intermediate I-2

Intermediate I-2 (22.0 g, Yield: 79%) was obtained in the same manner as in the 2nd to 7th steps of Synthesis Example 1 except that (5-chloro-2-fluorophenyl) boronic acid (53.43 g, 306.44 mmol) purchased from Merck & Co. Inc. was used instead of the (2-chloro-6-fluorophenyl) boronic acid.

### Synthesis Example 3: Synthesis of Intermediate I-3

Intermediate I-3 (20.0 g, Yield: 71%) was obtained in the same manner as in the 2nd to 7th steps of Synthesis Example 1 except that (3-chloro-2-fluorophenyl) boronic acid (53.43 g, 306.44 mmol) was used instead of the (2-chloro-6-fluorophenyl) boronic acid.

### Synthesis Example 4: Synthesis of Intermediate I-4

Intermediate I-4 (15.0 g, Yield: 54%) was obtained in the same manner as in the 1st to 7th steps of Synthesis Example 1 except that 2-amino-6-bromo-4-chlorophenol (40.0 g, 376.61 mmol) instead of the 2-amino-6-bromophenol and (2-fluorophenyl) boronic acid (60.63 g, 218.88 mmol) instead of the (2-chloro-6-fluorophenyl) boronic acid were used.

### Synthesis Example 5: Synthesis of Intermediate I-5

Intermediate I-5 (18.0 g, Yield: 64%) was obtained in the same manner as in the 1st to 7th steps of Synthesis Example 1 except that 2-amino-3-bromophenol (100.0 g, 485.83 mmol) was used instead of the 2-amino-6-bromophenol.

### Synthesis Example 6: Synthesis of Intermediate I-6

Intermediate I-6 (22.0 g, Yield: 79%) was obtained in the same manner as in the 1st to 7th steps of Synthesis Example 1 except that 2-amino-3-bromophenol (100.0 g, 485.83 mmol) instead of the 2-amino-6-bromophenol and (5-chloro-2-fluorophenyl) boronic acid (53.43 g, 306.44 mmol) instead of the (2-chloro-6-fluorophenyl) boronic acid were used.

Compounds of Synthesis Examples 7 to 23 were respectively synthesized in the same manner as in the 7^{th} step of Synthesis Example 1 except that Intermediate 1 (Intermediates I-1 to I-4) and a triazine intermediate (Intermediates A to J purchased from Sigma-Aldrich Co. Ltd., TCI Inc., Tokyo chemical industry, or P&H tech Co., Ltd.) were respectively used. Specific combinations of the intermediates are shown in Table 1.

TABLE 1

| Synthesis | Intermediate | Intermediate | Final | Amount | |
|---|---|---|---|---|---|
| Example | 1 | 2 | product | (yield) | Properties of final product (LC/MS)) |
| 7 | I-2 | A | Compound | 21.0 g | C40H24N4OS Exact Mass: 608.17 |
| | | | 11 | (71%) | found for 608.75 [M + H] |
| 8 | I-3 | A | Compound | 18.0 g | C40H24N4OS Exact Mass: 608.17 |
| | | | 13 | (64%) | found for 608.71 [M + H] |
| 9 | I-4 | A | Compound | 15.0 g | C40H24N4OS Exact Mass: 608.17 |
| | | | 14 | (50%) | found for 608.58 [M + H] |
| 10 | I-2 | B | Compound | 23.0 g | C46H28N4OS Exact Mass: 684.20 |
| | | | 20 | (71%) | found for 685.74 [M + H] |
| 11 | I-2 | C | Compound | 22.0 g | C40H22N4O2S Exact Mass: 622.15 |
| | | | 34 | (74%) | found for 622.57 [M + H] |
| 12 | I-1 | D | Compound | 20.0 g | C48H28N4OS Exact Mass: 708.20 |
| | | | 94 | (66%) | found for708.98 [M + H] |
| 13 | I-2 | E | Compound | 19.0 g | C48H28N4OS Exact Mass: 708.20 |
| | | | 98 | (63%) | found for 708.74 [M + H] |
| 14 | I-1 | F | Compound | 18.5 g | C46H26N4OS Exact Mass: 682.18 |
| | | | 111 | (63%) | found for 683.25 [M + H] |
| 15 | I-2 | F | Compound | 21.0 g | C46H26N4OS Exact Mass: 682.18 |
| | | | 116 | (72%) | found for 683.42 [M + H] |
| 16 | I-3 | F | Compound | 19.0 g | C46H26N4OS Exact Mass: 682.18 |
| | | | 121 | (65%) | found for 682.99 [M + H] |
| 17 | I-1 | I | Compound | 18.0 g | C46H27N5OS Exact Mass: 697.19 |
| | | | 133 | (62%) | found for 697.83 [M + H] |
| 18 | I-1 | H | Compound | 20.0 g | C46H27N5OS Exact Mass: 697.19 |
| | | | 134 | (67%) | found for698.24 [M + H] |
| 19 | I-2 | G | Compound | 22.0 g | C40H23N5OS Exact Mass: 621.16 |
| | | | 137 | (74%) | found for 622.08 [M + H] |
| 20 | I-2 | I | Compound | 23.0 g | C46H27N5OS Exact Mass: 697.19 |
| | | | 138 | (77%) | found for 697.85 [M + H] |
| 21 | I-1 | J | Compound | 22.0 g | C52H31N5OS Exact Mass: 773.22 |
| | | | 156 | (66%) | found for773.87 [M + H] |
| 22 | I-1 | A | Compound | 18.0 g | C40H24N4OS Exact Mass: 608.17 |
| | | | 72 | (69%) | found for 608.97 [M + H] |
| 23 | I-2 | A | Compound | 21.0 g | C40H24N4OS Exact Mass: 608.17 |
| | | | 77 | (71%) | found for 609.21 [M + H] |

### Synthesis Example 24: Synthesis of Compound Host 1

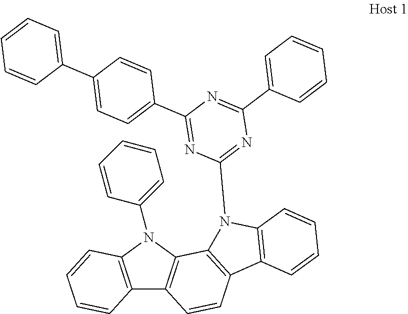 [SMILES: c1ccc(-c2ccc(-c3nc(-c4ccccc4)nc(-n4c5ccccc5c5ccc6c7ccccc7n(-c7ccccc7)c6c54)n3)cc2)cc1]

Compound Host 1 was synthesized by referring to the synthetic method of patent WO2016-194604.

HRMS (70 eV, EI+): m/z calcd for C45H29N5: 639.2423, found: 639.

Elemental Analysis: C, 84%; H, 5%

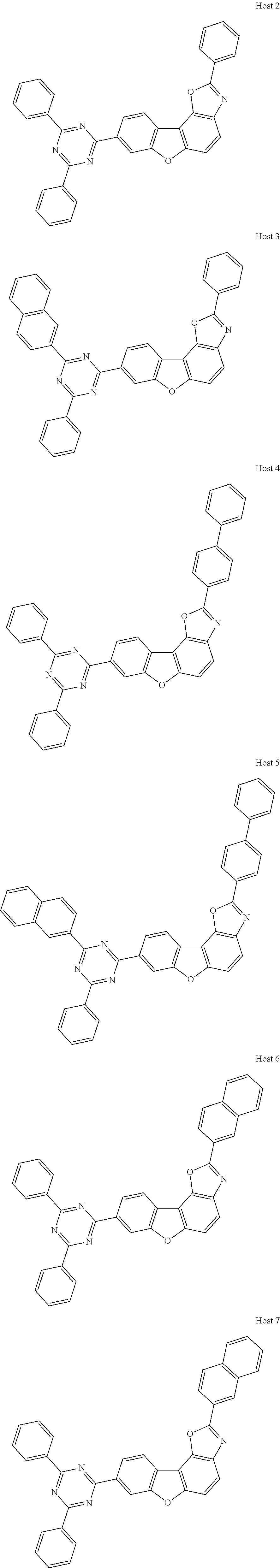 [SMILES: c1ccc(-c2nc(-c3ccccc3)nc(-c3ccc4c(c3)oc3ccc5nc(-c6ccccc6)oc5c34)n2)cc1 ; c1ccc(-c2nc(-c3ccc4ccccc4c3)nc(-c3ccc4c(c3)oc3ccc5nc(-c6ccccc6)oc5c34)n2)cc1 ; c1ccc(-c2ccc(-c3nc4ccc5oc6cc(-c7nc(-c8ccccc8)nc(-c8ccccc8)n7)ccc6c5c4o3)cc2)cc1 ; c1ccc(-c2ccc(-c3nc4ccc5oc6cc(-c7nc(-c8ccccc8)nc(-c8ccc9ccccc9c8)n7)ccc6c5c4o3)cc2)cc1 ; c1ccc(-c2nc(-c3ccccc3)nc(-c3ccc4c(c3)oc3ccc5nc(-c6ccc7ccccc7c6)oc5c34)n2)cc1 ; c1ccc(-c2nc(-c3ccccc3)nc(-c3ccc4c(c3)oc3ccc5nc(-c6ccc7ccccc7c6)oc5c34)n2)cc1]

### Synthesis Example 25: Synthesis of Compound Host 2

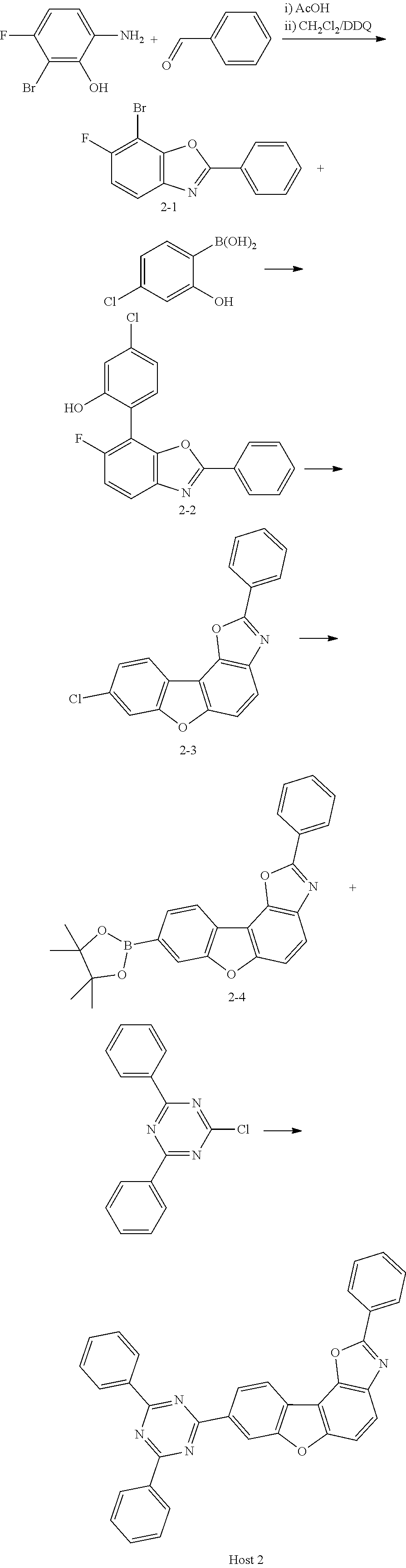

### 1 st Step: Synthesis of Intermediate 2-1

100 g (404.86 mmol) of 6-amino-2-bromo-3-fluorophenol (Merck & Co, Inc.) and 43 g (485.83 mmol) of benzaldehyde (Merck & Co, Inc.) were dissolved in 120 g (2024.29 mmol) of acetic acid and then, reacted at room temperature for 3 hours. Subsequently, the reactant was poured into a large amount of DIW (de-Ionized water) and then, stirred for 30 minutes, filtered, and twice or more washed with water. A solid was separated therefrom, extracted with dichloromethane and DIW, and dried with magnesium sulfate, and the solvents were all removed. Subsequently, the solid was dissolved in an amount corresponding to 10 times that of dichloromethane, and 84.9 g (374 mmol) of 2,3-dichloro-5,6-dicyano-p-benzoquinone (DDQ) was slowly added thereto and then, reacted at room temperature for 2 hours, obtaining Intermediate 2-1 (70 g, Yield: 70%).

### 2nd Step: Synthesis of Intermediate 2-2

Intermediate 2-1 (70.0 g, 239.64 mmol), (4-chloro-2-hydroxyphenyl) boronic acid (45.44 g, 263.60 mmol), Pd(PPh₃)₄ (13.85 g, 11.98 mmol), and K₂CO₃ (99.36 g, 718.91 mmol) were dissolved in dioxane (600 ml) and DIW (3000 ml) and then, refluxed by heating under a nitrogen atmosphere. After 12 hours, the reaction solution was cooled, and after removing an aqueous layer and also, removing the solvents with a rotary evaporator, extraction was performed with methylene chloride/DIW. The obtained organic layer was columned with hexane:EA=4:1 (v/v), obtaining Intermediate 2-2 (40.0 g, Yield: 49%).

### 3rd Step: Synthesis of Intermediate 2-3

Intermediate 2-2 (40.0 g, 117.73 mmol) and K₃PO₄ (50.0 g, 235.47 mmol) were dissolved in DMF (N,N-dimethylformamide) (200 ml) and then, heated under reflux at 120° C. for 3 hours. When a reaction was completed, after removing the solvent with a rotary evaporator, an organic layer, which was extracted with methylene chloride/DIW, was dried with MgSO₄ concentrated and then, stirred with a small amount of methanol to obtain a solid, which was recrystallized with 200 mL of toluene, obtaining Intermediate 2-3 (30.0 g, Yield: 80%).

### 4th Step: Synthesis of Intermediate 2-4

30.0 g (93.83 mmol) of Intermediate 2-3, 24.0 g (93.83 mmol) of bis(pinacolato)diboron, 5.16 g (5.63 mmol) of Pd₂(dba)₃, 6.31 g (22.52 mmol) of P(Cy)₃, and 27.63 g (281.48 mmol) of KOAc were dissolved in 300 m1 of xylene and then, stirred under reflux at 150° C. for 12 hours. When a reaction was completed, after removing the reaction solvent with a rotary evaporator, an organic layer, which was extracted with methylene chloride, was columned with hexane:EA=4:1 (v/v), obtaining intermediate 2-4 (30.0 g, Yield: 78%).

### 5th Step: Synthesis of Host 2

In a round-bottomed flask, 30.0 g (71.90 mmol) of Intermediate 2-4, 17.5 g (65.37 mmol) of 2-chloro-4,6-diphenyl-1,3,5-triazine purchased from P&H Tech Co., Ltd. (http://www.phtech.co.kr/), 3.78 g (3.27 mmol) of Pd(PPh₃)₄, and 22.59 g (163.42 mmol) of K₂CO₃ were dissolved in 120 mL of THF and 60 mL of distilled water and then, refluxed by heating under a nitrogen atmosphere. After 12 hours, the reaction solution was cooled, and after removing an aqueous layer, an organic layer therefrom was dried under a reduced pressure. The obtained solid was washed with water and methanol and twice recrystallized with 200 mL of toluene, obtaining Host 2 (28.0 g, Yield: 83%). LC/MS calculated for: C34H20N4O2 Exact Mass 516.16 found for 517.41 [M+H].

### Synthesis Example 26: Synthesis of Compound Host 3

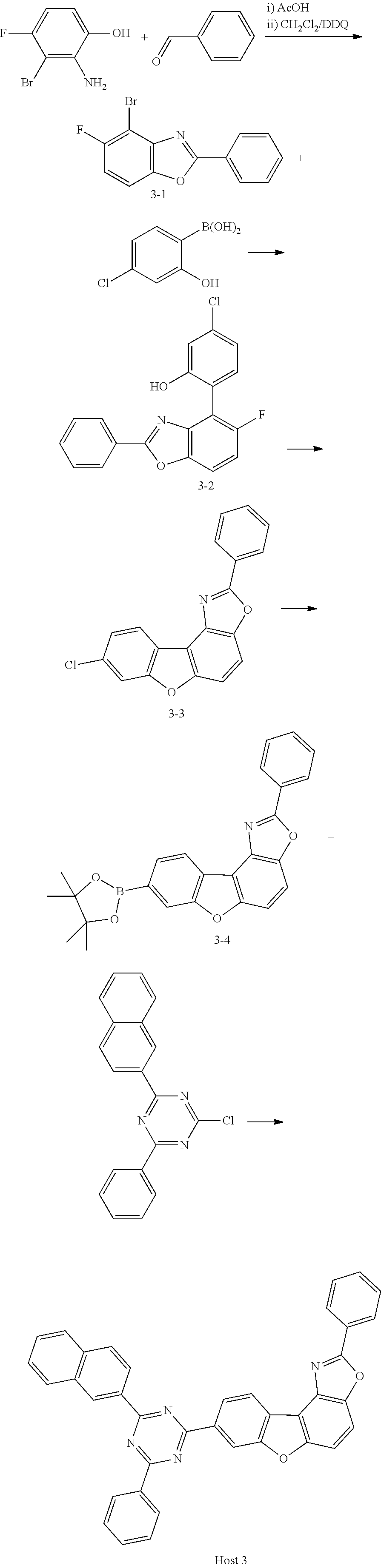

100 g (404.86 mmol) of 2-amino-3-bromo-4-fluorophenol (Merck & Co., Inc.) instead of the 6-amino-2-bromo-3-fluorophenol was used obtained in the same manner as in the 1st to 4th steps of Synthesis Example 25 to obtain Intermediate 3-4 (30 g, 65.14 mmol), which was used with 2-chloro-4-(naphthalen-2-yl)-6-phenyl-1,3,5-triazine (20.7 g, 65.14 mmol) purchased from P&H Tech Co., Ltd. (http://www.phtech.co.kr/), obtaining Host 3 (30.0 g, Yield: 81%). LC/MS calculated for: C38H22N4O2 Exact Mass 566.17 found for 567.01 [M+H].

### Synthesis Example 27: Synthesis of Compound Host 4

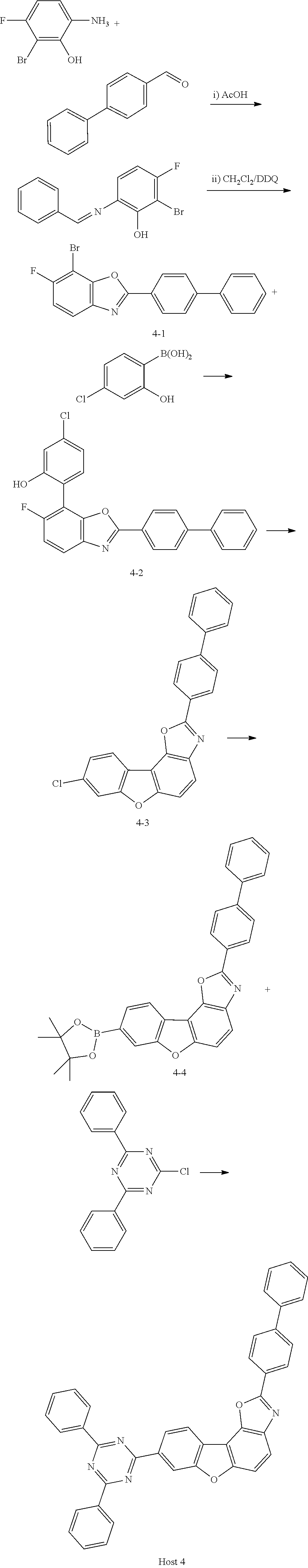

[1,1′-biphenyl]-4-carbaldehyde (74 g, 406.10 mmol) instead of the benzaldehyde was used in the same manner as in the 1st to 4th steps of Synthesis Example 25 to obtain Intermediate 4-4 (40 g, 80.94 mmol), which was used with 2-chloro-4,6-diphenyl-1,3,5-triazine (18.2 g, 43.4 mmol) purchased from P&H Tech Co. Ltd. (http://www.phtech.co.kr/), obtaining Host 4 (30.0 g, Yield: 69%).

LC/MS calculated for: C40H24N4O2 Exact Mass 592.19 found for 592.97 [M+H].

### Synthesis Example 28: Synthesis of Compound Host 5

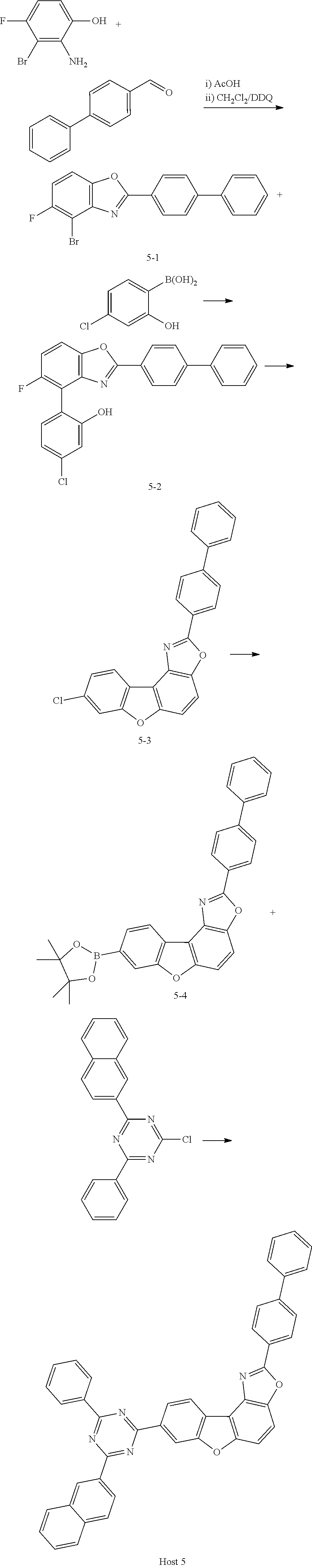

100 g (404.86 mmol of 2-amino-3-bromo-4-fluorophenol (Merck&Co., Inc.) instead of the 6-amino-2-bromo-3-fluorophenol and [1,1′-biphenyl]-4-carbaldehyde (74 g, 406.10 mmol) instead of the benzaldehyde were used in the same manner as in the 1^{st} to 4^{th} steps of Synthesis Example 25 to obtain Intermediate 5-4 (40 g, 80.94 mmol), which was used with 2-chloro-4-(naphthalen-2-yl)-6-phenyl-1,3,5-triazine (23.0 g, 72.38 mmol) purchased from P&H Tech Co., Ltd. (http://www.phtech.co.kr/), obtaining Host 5 (34.0 g, Yield: 73%). LC/MS calculated for: C44H26N4O26 Exact Mass 642.21 found for 643.05 [M+H].

### Synthesis Example 29: Synthesis of Compound Host 6

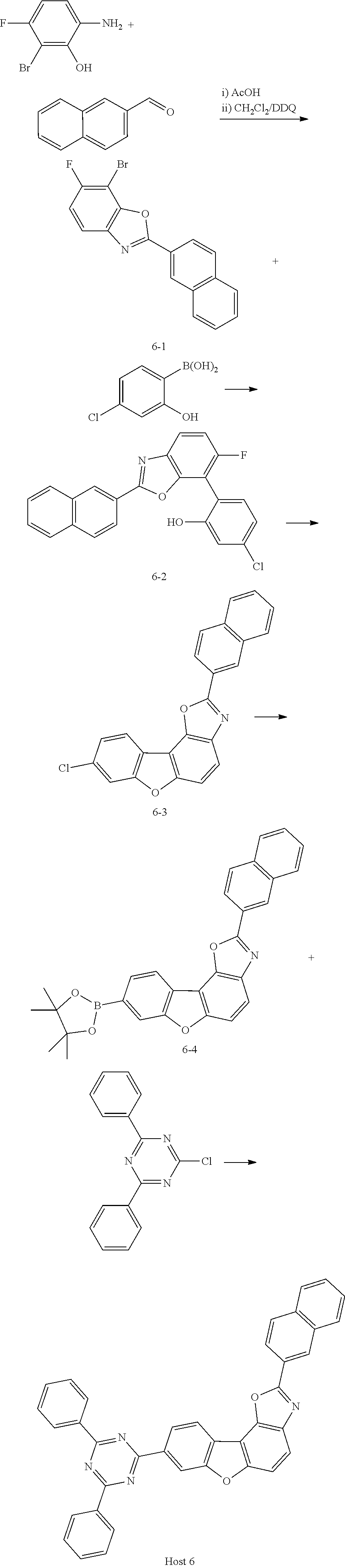

2-naphthaldehyde (63 g, 403.38 mmol) instead of the benzaldehyde was used in the same manner as in the 1^{st} to 4^{th} steps of Synthesis Example 25 to obtain Intermediate 6-4 (40 g, 86.28 mmol), which was used with 2-chloro-4,6-diphenyl-1,3,5-triazine (21.0 g, 78.44 mmol) purchased from P&H Tech Co., Ltd. (http://www.phtech.co.kr/), obtaining Host 6 (36.0 g, Yield: 71%). LC/MS calculated for: C38H22N4O2 Exact Mass 566.17 found for 567.22 [M+H].

### Synthesis Example 30: Synthesis of Compound Host 7

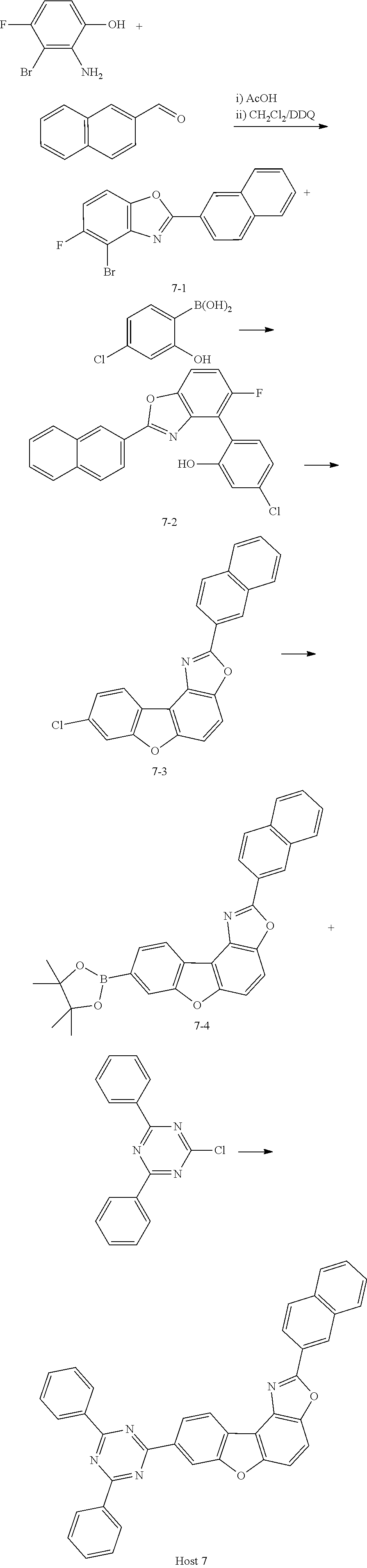

100 g (404.86 mmol) of 2-amino-3-bromo-4-fluorophenol (Merck & Co., Inc.) instead of the 6-amino-2-bromo-3-fluorophenol and 2-naphthaldehyde (63 g, 403.38 mmol) instead of the benzaldehyde were used in the same manner as in the 1^{st} to 4^{th} steps of Synthesis Example 25 to obtain Intermediate 7-4 (40 g, 86.28 mmol), which was used with 2-chloro-4,6-diphenyl-1,3,5-triazine (21.0 g, 78.44 mmol) purchased from P&H Tech Co., Ltd. (http://www.phtech.co.kr/), obtaining Host 7 (36.0 g, Yield: 71%). LC/MS calculated for: C38H22N4O2 Exact Mass 566.17 found for 567.62 [M+H].

### (Manufacturing of Organic Optoelectronic Device)

### Example 1

A glass substrate coated with ITO (Indium tin oxide) was washed with distilled water and ultrasonic waves. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited to on the hole injection layer to form a 1350 Å-thick hole transport layer. Compound B was deposited on the hole transport layer to form a 350 Å-thick hole transport auxiliary layer, and Compound 10 synthesized in Synthesis Example 1 was used as a host on the hole transport auxiliary layer and PhGD was doped as a dopant at 7 wt % to form a 400 Å-thick light emitting layer by vacuum deposition. Subsequently, Compound C was deposited on the light emitting layer to a thickness of to form a 50 Å-thick electron transport auxiliary layer, and Compound D and Liq were simultaneously vacuum-deposited at a weight ratio of 1:1 to form a 300 Å-thick electron transport layer. LiQ (15 Å) and Al (1200 Å) were sequentially vacuum-deposited on the electron transport layer to form a cathode, thereby manufacturing an organic light emitting diode.

The organic light emitting diode has a structure of ITO/Compound A (3% NDP-9 doping, 100 Å)/Compound A (1350 Å)/Compound B (350 Å)/EML [Compound 10 (93 wt %):PhGD (7 wt %)] (400 Å)/Compound C (50 Å)/Compound D:LiQ (300 Å)/LiQ (15 Å)/Al (1200 Å).
Compound A: N-(biphenyl-4-yl)-9,9-dimethyl-N-(4-(9-phenyl-9H-carbazol-3-yl)phenyl)-9H-fluoren-2-amine
Compound B: N-[4-(4-Dibenzofuranyl)phenyl]-N-[4-(9-phenyl-9H-fluoren-9-yl)phenyl][1,1′-biphenyl]-4-amine
Compound C: 2,4-Diphenyl-6-(4′,5′,6′-triphenyl [1,1′:2′,1″:3″,1′:3′,1′″-quinquephenyl]-3″-yl)-1,3,5-triazine
Compound D: 2-(1,1′-Biphenyl-4-yl)-4-(9,9-diphenylfluoren-4-yl)-6-phenyl-1,3,5-triazine

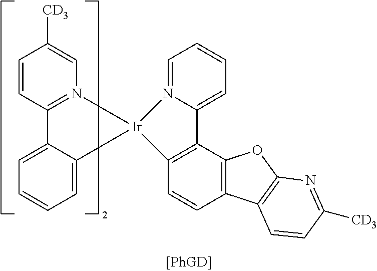

### Examples 2 to 18 and Comparative Examples 1 to 7

Each organic light emitting diode was manufactured in the same manner as in Example 1, except that the composition was changed to that described in Table 2.

### Evaluation

The driving voltage, luminous efficiency, and life-span characteristics of the organic light emitting diodes according to Examples 1 to 18 and Comparative Examples 1 to 7 were evaluated.

Specific measurement methods are as follows, and the results are shown in Table 2.

### (1) Measurement of Current Density Change Depending on Voltage Change

The obtained organic light emitting diodes were measured regarding a current value flowing in the unit diode, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

The current efficiency (cd/A) at the same current density (10 mA/cm²) was calculated using the luminance and current density measured from (1) and (2) above and the voltage.

The luminous efficiency values of Examples 1 to 18 and Comparative Examples 1 to 7 were calculated as relative values based on Comparative Example 1 and are listed in Table 2.

### (4) Measurement of Life-Span

The results were obtained by measuring a time when current efficiency (cd/A) was decreased down to 95%, while luminance (cd/m2) was maintained to be 24000 cd/m².

The life-span measurement values of Examples 1 to 18 and Comparative Examples 1 to 7 were calculated as relative values based on Comparative Example 1 and are listed in Table 2.

### (5) Measurement of Driving Voltage

The results were obtained by measuring the driving voltage of each diode at 15 mA/cm² using a current-voltage meter (Keithley 2400).

The driving voltages of Examples 1 to 18 and Comparative Examples 1 to 7 were calculated as relative values based on Comparative Example 1 and are listed in Table 2.

TABLE 2

| | | Driving | Efficiency | T95 life-span |
|---|---|---|---|---|
| No. | Host | voltage (%) | (%) | (%) |
| Example 1 | Compound 10 | 75 | 210 | 100 |
| Example 2 | Compound 11 | 80 | 200 | 120 |
| Example 3 | Compound 13 | 90 | 210 | 110 |
| Example 4 | Compound 14 | 100 | 180 | 150 |
| Example 5 | Compound 20 | 75 | 180 | 120 |
| Example 6 | Compound 34 | 80 | 185 | 115 |
| Example 7 | Compound 94 | 80 | 150 | 100 |
| Example 8 | Compound 98 | 90 | 170 | 100 |
| Example 9 | Compound 111 | 75 | 190 | 140 |
| Example 10 | Compound 116 | 80 | 180 | 150 |
| Example 11 | Compound 121 | 92 | 187 | 130 |
| Example 12 | Compound 133 | 95 | 160 | 150 |
| Example 13 | Compound 134 | 90 | 180 | 150 |
| Example 14 | Compound 137 | 100 | 155 | 170 |
| Example 15 | Compound 138 | 90 | 150 | 175 |
| Example 16 | Compound 156 | 98 | 185 | 160 |
| Example 17 | Compound 72 | 75 | 220 | 95 |
| Example 18 | Compound 77 | 80 | 210 | 110 |
| Comparative | Host 1 | 100 | 100 | 100 |
| Example 1 | | | | |
| Comparative | Host 2 | 85 | 104 | 50 |
| Example 2 | | | | |
| Comparative | Host 3 | 88 | 70 | 15 |
| Example 3 | | | | |
| Comparative | Host 4 | 85 | 100 | 48 |
| Example 4 | | | | |
| Comparative | Host 5 | 93 | 72 | unmeasurable |
| Example 5 | | | | |
| Comparative | Host 6 | 94 | 68 | 10 |
| Example 6 | | | | |
| Comparative | Host 7 | 90 | 65 | unmeasurable |
| Example 7 | | | | |

Referring to Table 2, it can be confirmed that the organic light emitting diodes according to Examples 1 to 18 have significantly improved driving voltage, luminous efficiency, and/or life-span characteristics compared to the organic light emitting diodes according to Comparative Examples 1 to 7.

In particular, Comparative Examples 5 and 7 in Table 2 had low Tl energy levels, so that the organic light emitting diodes using the compounds as hosts died suddenly and lifespan measurements were not possible.

1. A compound for an organic optoelectronic device represented by a combination of Chemical Formula 1 and Chemical Formula 2: 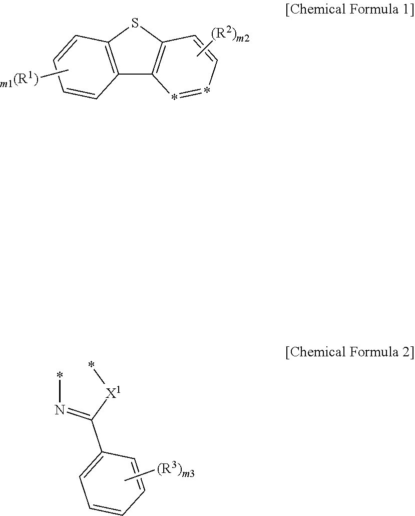
wherein, in Chemical Formula 1 and Chemical Formula 2,
X¹ is O or S,
*s are each linking carbon (C),
* in Chemical Formula 1 is linked to * in Chemical Formula 2, respectively,
R¹ and R² are each independently hydrogen, deuterium, a substituted or unsubstituted C1 to C30 alkyl group, a substituted or unsubstituted C6 to C30 aryl group, or a substituted or unsubstituted C2 to C30 heterocyclic group,
R³ is hydrogen, deuterium, a substituted or unsubstituted phenyl group, or a group represented by Chemical Formula a,
m1 is one of integers of 1 to 4,
m2 is an integer of 1 or 2,
m3 is one of integers of 1 to 5, and
at least one of R¹ to R³ is a group represented by Chemical Formula a, 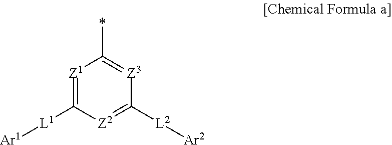
wherein, in Chemical Formula a,
Z¹ to Z³ are each independently N or C-L^{a}-R^{a},
at least two of Z¹ to Z³ are N,
L^{a}, L¹, and L² are each independently a single bond, a substituted or unsubstituted C6 to C30 arylene group, or a substituted or unsubstituted C2 to C30 heterocyclic group,
R^{a} is hydrogen, deuterium, a substituted or unsubstituted C1 to C30 alkyl group, a substituted or unsubstituted C6 to C30 aryl group, or a substituted or unsubstituted C2 to C30 heterocyclic group, and
Ar¹ and Ar² are each independently a substituted or unsubstituted C6 to C30 aryl group or a substituted or unsubstituted C2 to C30 heterocyclic group.

2. The compound for the organic optoelectronic device as claimed in claim 1, wherein
the combination of Chemical Formula 1 and Chemical Formula 2 is represented by Chemical Formula 1A or Chemical Formula 1B: 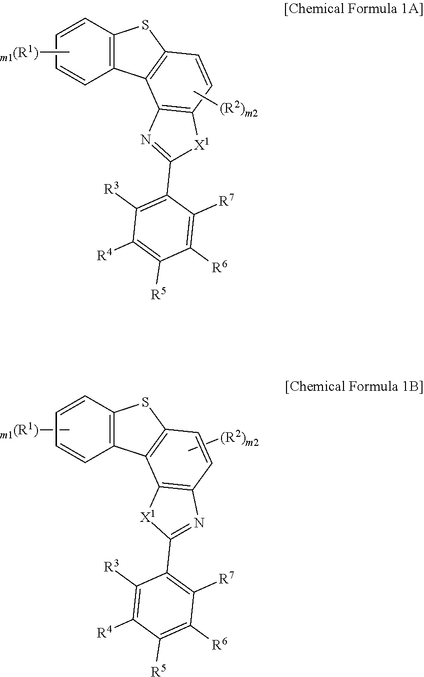
wherein, in Chemical Formula 1A to Chemical Formula 1B, X¹, R¹ to R³, and m1 to m3 are defined the same as those of Chemical Formula 1 and Chemical Formula 2.

3. The compound for the organic optoelectronic device as claimed in claim 1, wherein
the combination of Chemical Formula 1 and Chemical Formula 2 is represented by any one of Chemical Formula 1A-I, Chemical Formula 1A-II, Chemical Formula 1B-I, Chemical Formula 1B-II, Chemical Formula 1C-I, and Chemical Formula 1C-II: 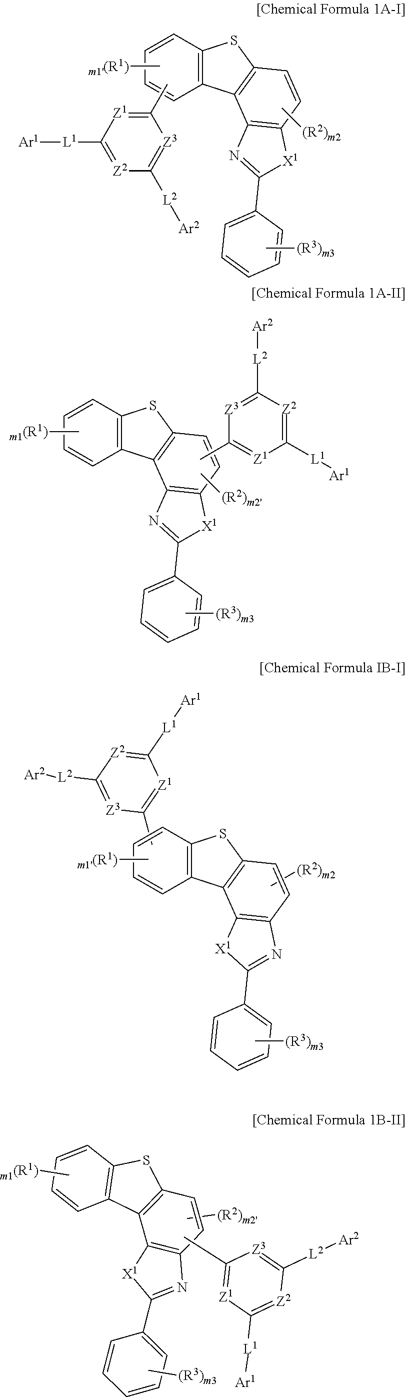 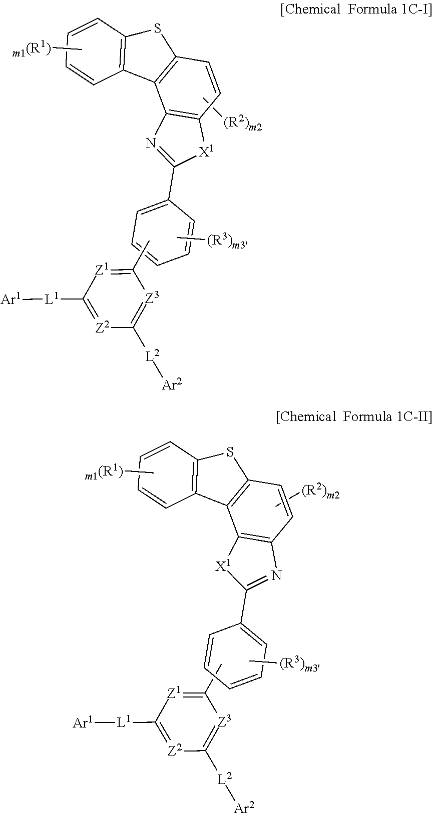
wherein, in Chemical Formula 1A-I, Chemical Formula 1A-II, Chemical Formula 1B-I, Chemical Formula 1B-II, Chemical Formula 1C-I, and Chemical Formula 1C-II,
X¹, Z¹ to Z³, Ar¹, Ar², L¹, L², and m1 to m3 are defined the same as those of Chemical Formula 2 and Chemical Formula a,
R¹ and R² are each independently hydrogen, deuterium, a substituted or unsubstituted C1 to C30 alkyl group, a substituted or unsubstituted C6 to C30 aryl group, or a substituted or unsubstituted C2 to C30 heterocyclic group,
R³ is hydrogen, deuterium, or a substituted or unsubstituted phenyl group,
m1′ is one of integers of 1 to 3,
m2′ is 1, and
m3′ is one of integers of 1 to 4.

4. The compound for the organic optoelectronic device as claimed in claim 3, wherein the combination of Chemical Formula 1 and Chemical Formula 2 is represented by any one of Chemical Formula 1A-I, Chemical Formula 1A-II, Chemical Formula 1B-I, and Chemical Formula 1B-II.

5. The compound for the organic optoelectronic device as claimed in claim 3, wherein the combination of Chemical Formula 1 and Chemical Formula 2 is represented by any one of Chemical Formula 1A-I-1 to Chemical Formula 1A-I-4, Chemical Formula 1A-II-1, Chemical Formula 1B-I-1 to Chemical Formula 1B-I-4, and Chemical Formula 1B-II-1: 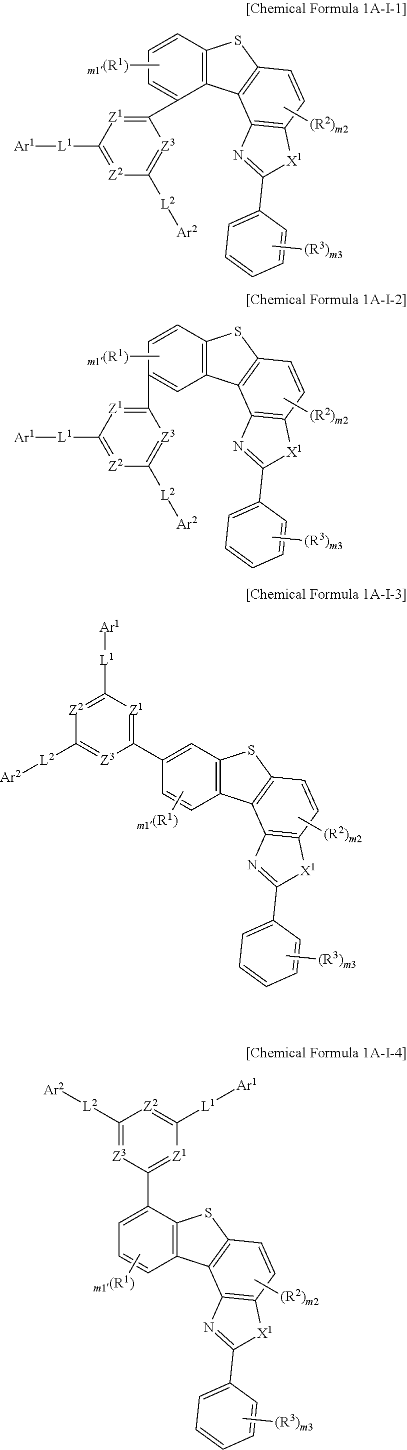 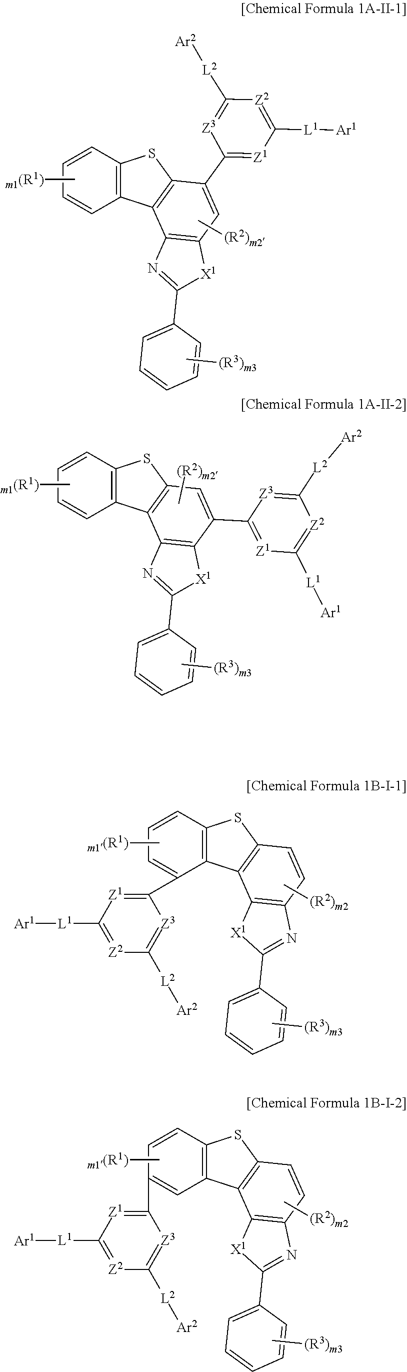 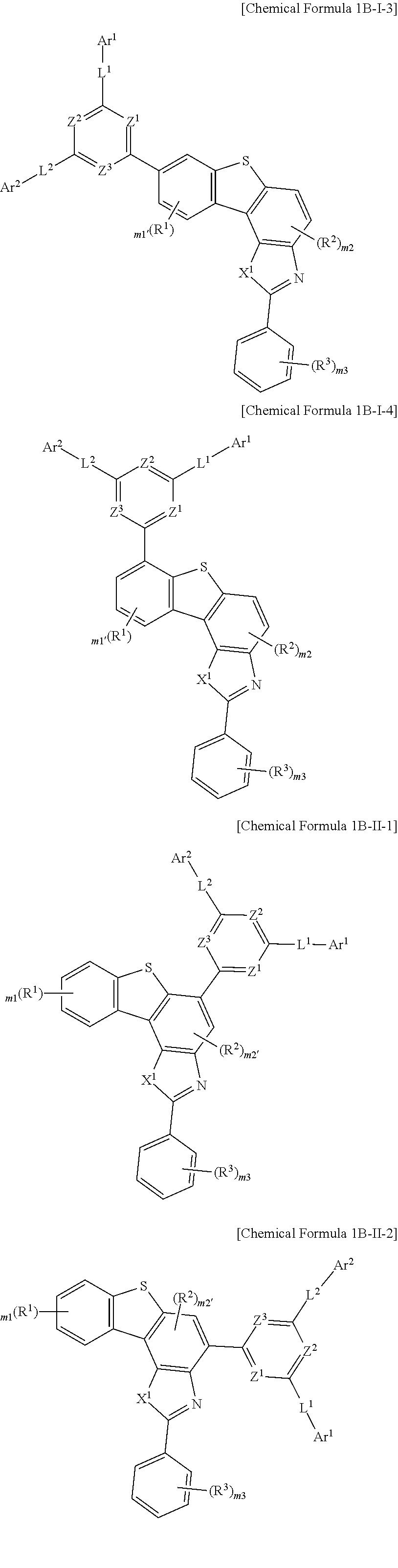
in Chemical Formula 1A-I-1 to Chemical Formula 1A-I-4, Chemical Formula 1A-II-1, Chemical Formula 1B-I-1 to Chemical Formula 1B-I-4, and Chemical Formula 1B-II-1, X¹, Z¹ to Z³, R¹ to R³, Ar¹, Ar², L¹, L², m1, m1′, m2, m2′, and m3 are defined the same as those of Chemical Formula 1A-I, Chemical Formula 1A-II, Chemical Formula 1B-I, Chemical Formula 1B-II, Chemical Formula 1C-I, and Chemical Formula 1C-II.

6. The compound for the organic optoelectronic device as claimed in claim 1, wherein Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, a substituted or unsubstituted dibenzosilolyl group, or a substituted or unsubstituted indolocarbazolyl group.

7. The compound for the organic optoelectronic device as claimed in claim 1, wherein L¹-Ar¹ and L²-Ar² are each independently selected from the substituents listed in Group I: 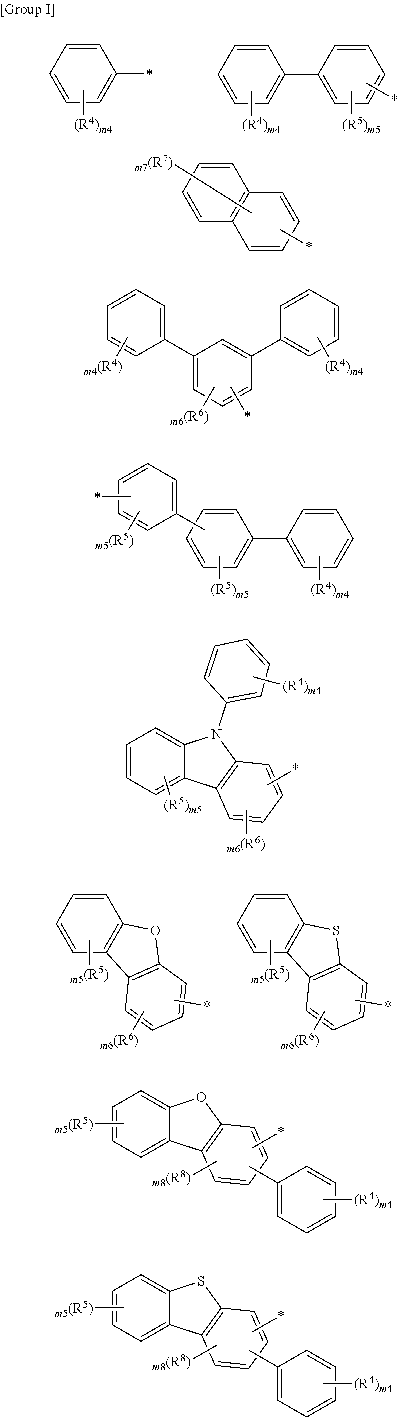 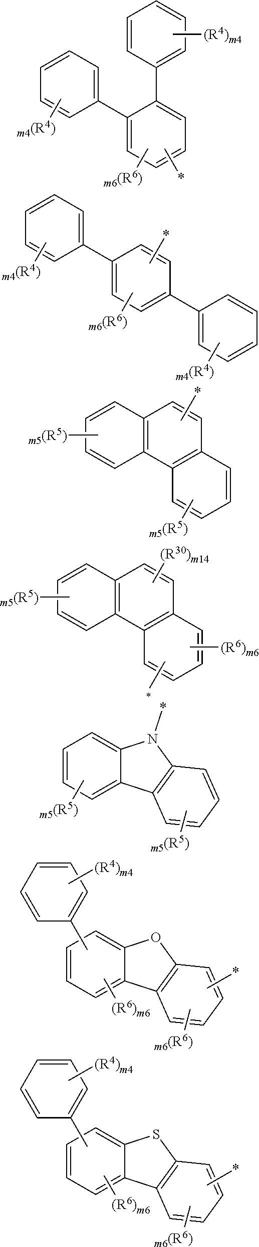 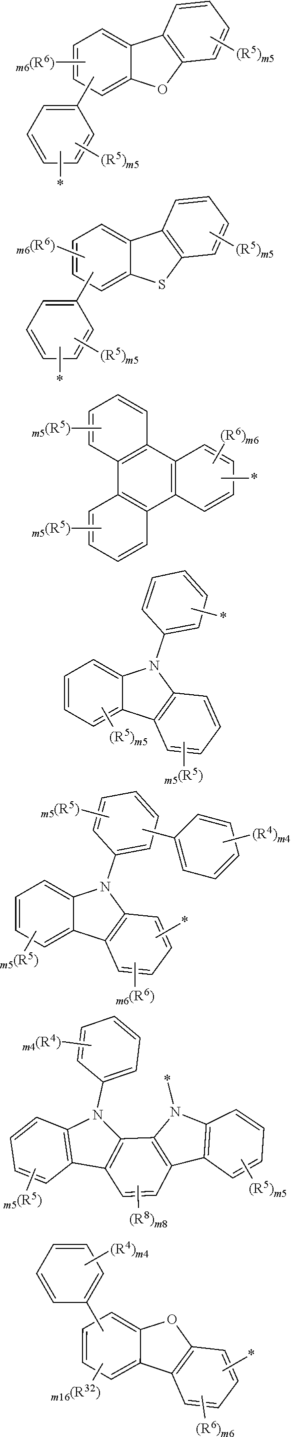 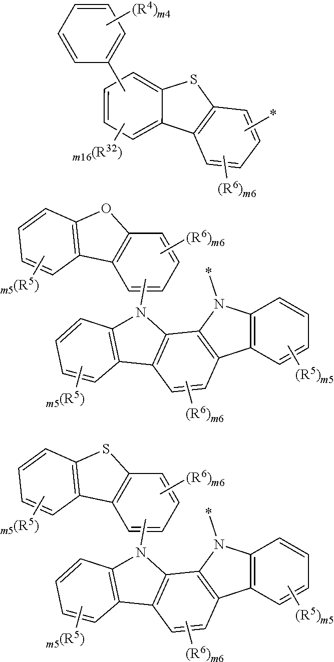
wherein, in Group I,
R⁴ to R⁷ are each independently hydrogen, deuterium, a cyano group, a C1 to C10 alkyl group, or a C6 to C12 aryl group,
m4 is one of integers of 1 to 5,
m5 is one of integers of 1 to 4,
m6 is one of integers of 1 to 3,
m7 is one of integers of 1 to 7,
m8 is an integer of 1 or 2, and
* is a linking point.

8. The compound for the organic optoelectronic device as claimed in claim 1, wherein the compound is selected from compounds listed in Group 1:

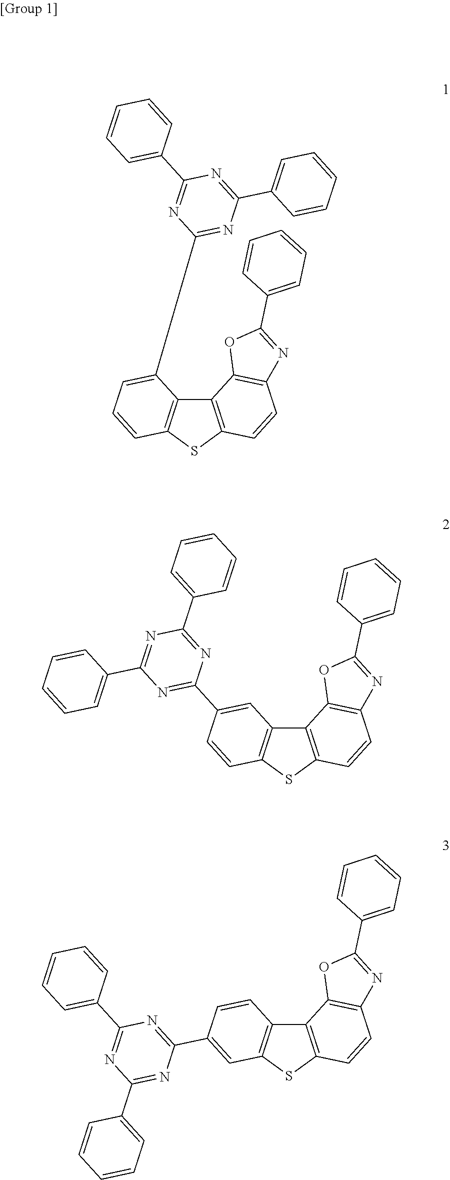 [SMILES: c1ccc(-c2nc(-c3ccccc3)nc(-c3cccc4sc5ccc6nc(-c7ccccc7)oc6c5c34)n2)cc1 ; c1ccc(-c2nc(-c3ccccc3)nc(-c3ccc4sc5ccc6nc(-c7ccccc7)oc6c5c4c3)n2)cc1 ; c1ccc(-c2nc(-c3ccccc3)nc(-c3ccc4c(c3)sc3ccc5nc(-c6ccccc6)oc5c34)n2)cc1]

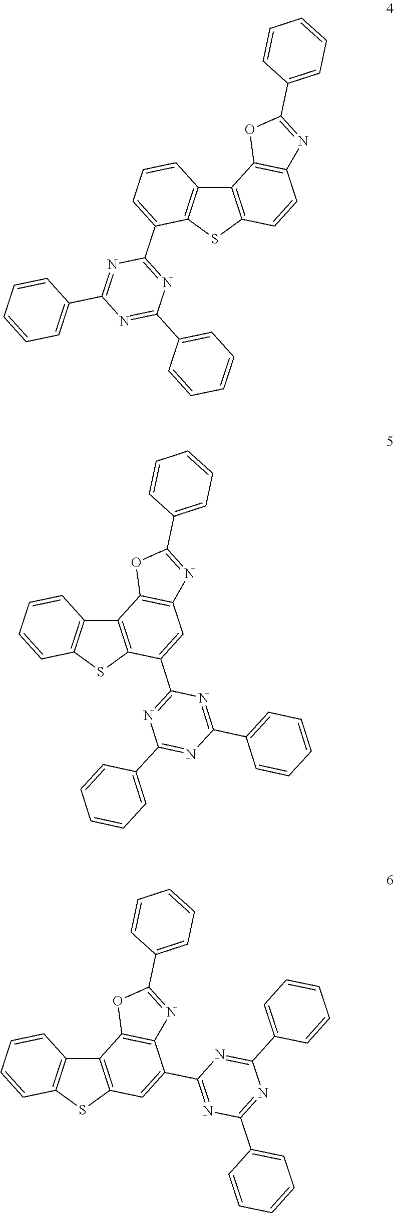 [SMILES: c1ccc(-c2nc(-c3ccccc3)nc(-c3cccc4c3sc3ccc5nc(-c6ccccc6)oc5c34)n2)cc1 ; c1ccc(-c2nc(-c3ccccc3)nc(-c3cc4nc(-c5ccccc5)oc4c4c3sc3ccccc34)n2)cc1 ; c1ccc(-c2nc(-c3ccccc3)nc(-c3cc4sc5ccccc5c4c4oc(-c5ccccc5)nc34)n2)cc1]

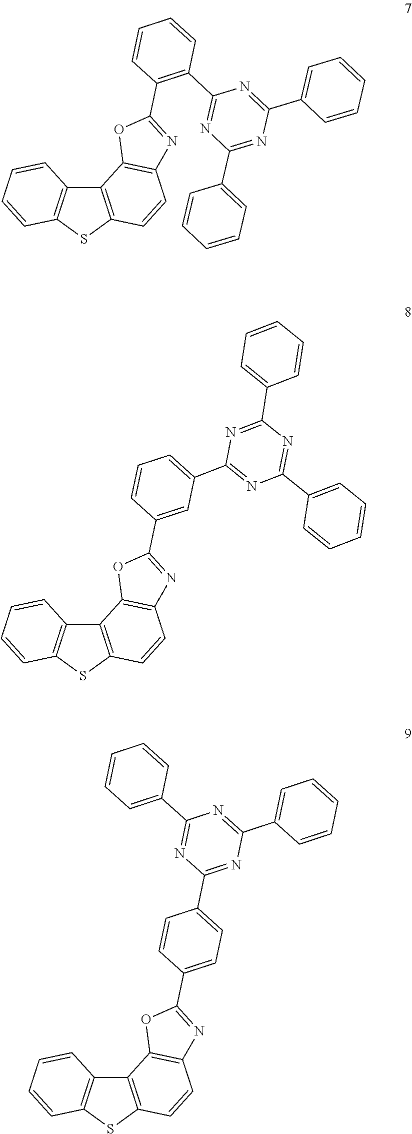 [SMILES: c1ccc(-c2nc(-c3ccccc3)nc(-c3ccccc3-c3nc4ccc5sc6ccccc6c5c4o3)n2)cc1 ; c1ccc(-c2nc(-c3ccccc3)nc(-c3cccc(-c4nc5ccc6sc7ccccc7c6c5o4)c3)n2)cc1 ; c1ccc(-c2nc(-c3ccccc3)nc(-c3ccc(-c4nc5ccc6sc7ccccc7c6c5o4)cc3)n2)cc1]

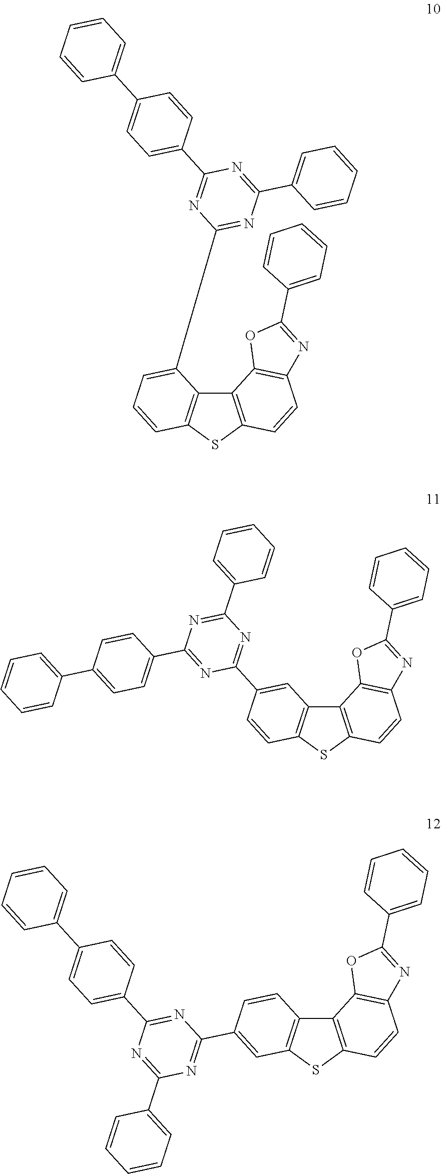 [SMILES: c1ccc(-c2ccc(-c3nc(-c4ccccc4)nc(-c4cccc5sc6ccc7nc(-c8ccccc8)oc7c6c45)n3)cc2)cc1 ; c1ccc(-c2ccc(-c3nc(-c4ccccc4)nc(-c4ccc5sc6ccc7nc(-c8ccccc8)oc7c6c5c4)n3)cc2)cc1 ; c1ccc(-c2ccc(-c3nc(-c4ccccc4)nc(-c4ccc5c(c4)sc4ccc6nc(-c7ccccc7)oc6c45)n3)cc2)cc1]

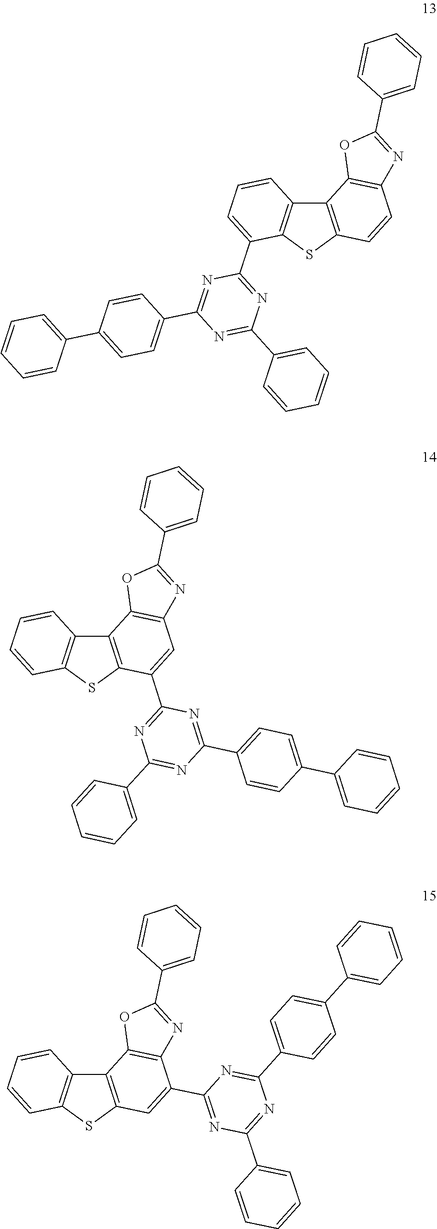 [SMILES: c1ccc(-c2ccc(-c3nc(-c4ccccc4)nc(-c4cccc5c4sc4ccc6nc(-c7ccccc7)oc6c45)n3)cc2)cc1 ; c1ccc(-c2ccc(-c3nc(-c4ccccc4)nc(-c4cc5nc(-c6ccccc6)oc5c5c4sc4ccccc45)n3)cc2)cc1 ; c1ccc(-c2ccc(-c3nc(-c4ccccc4)nc(-c4cc5sc6ccccc6c5c5oc(-c6ccccc6)nc45)n3)cc2)cc1]

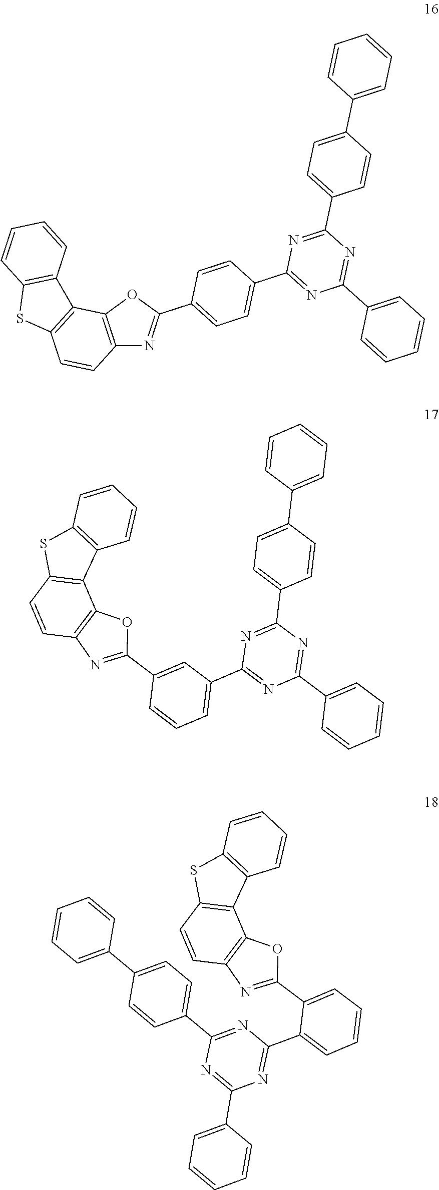 [SMILES: c1ccc(-c2ccc(-c3nc(-c4ccccc4)nc(-c4ccc(-c5nc6ccc7sc8ccccc8c7c6o5)cc4)n3)cc2)cc1 ; c1ccc(-c2ccc(-c3nc(-c4ccccc4)nc(-c4cccc(-c5nc6ccc7sc8ccccc8c7c6o5)c4)n3)cc2)cc1 ; c1ccc(-c2ccc(-c3nc(-c4ccccc4)nc(-c4ccccc4-c4nc5ccc6sc7ccccc7c6c5o4)n3)cc2)cc1]

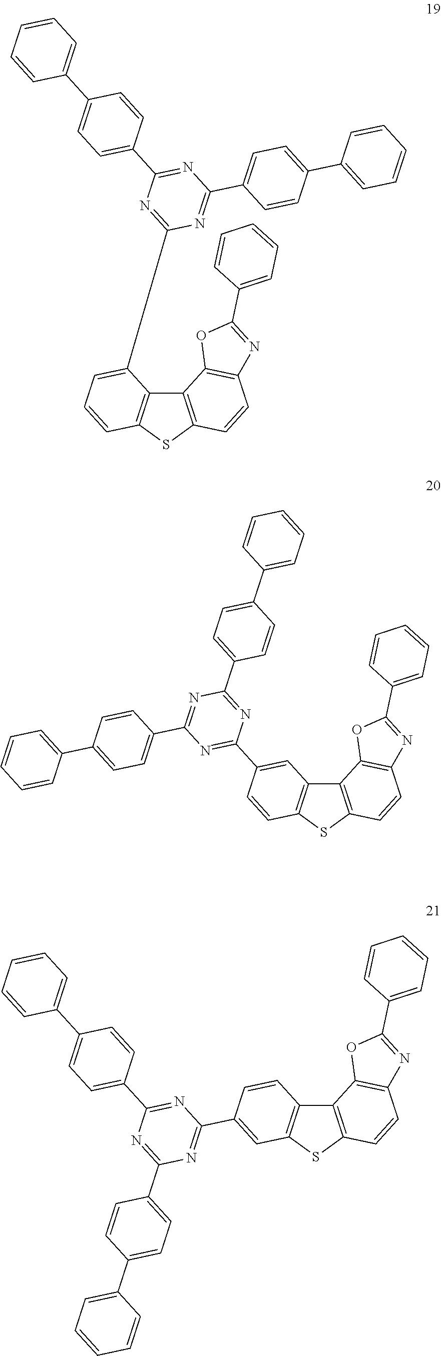 [SMILES: c1ccc(-c2ccc(-c3nc(-c4ccc(-c5ccccc5)cc4)nc(-c4cccc5sc6ccc7nc(-c8ccccc8)oc7c6c45)n3)cc2)cc1 ; c1ccc(-c2ccc(-c3nc(-c4ccc(-c5ccccc5)cc4)nc(-c4ccc5sc6ccc7nc(-c8ccccc8)oc7c6c5c4)n3)cc2)cc1 ; c1ccc(-c2ccc(-c3nc(-c4ccc(-c5ccccc5)cc4)nc(-c4ccc5c(c4)sc4ccc6nc(-c7ccccc7)oc6c45)n3)cc2)cc1]

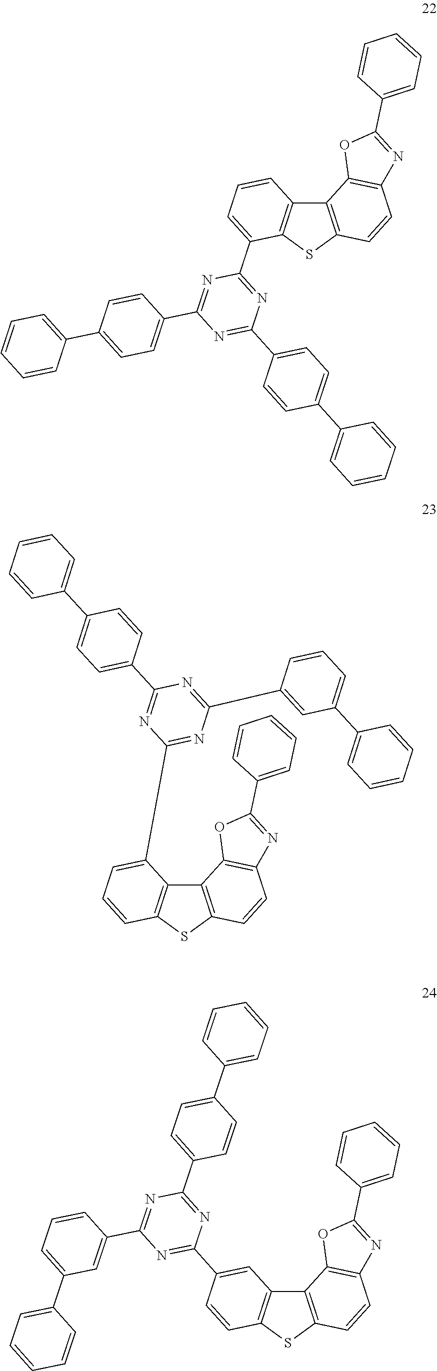 [SMILES: c1ccc(-c2ccc(-c3nc(-c4ccc(-c5ccccc5)cc4)nc(-c4cccc5c4sc4ccc6nc(-c7ccccc7)oc6c45)n3)cc2)cc1 ; c1ccc(-c2ccc(-c3nc(-c4cccc(-c5ccccc5)c4)nc(-c4cccc5sc6ccc7nc(-c8ccccc8)oc7c6c45)n3)cc2)cc1 ; c1ccc(-c2ccc(-c3nc(-c4cccc(-c5ccccc5)c4)nc(-c4ccc5sc6ccc7nc(-c8ccccc8)oc7c6c5c4)n3)cc2)cc1]

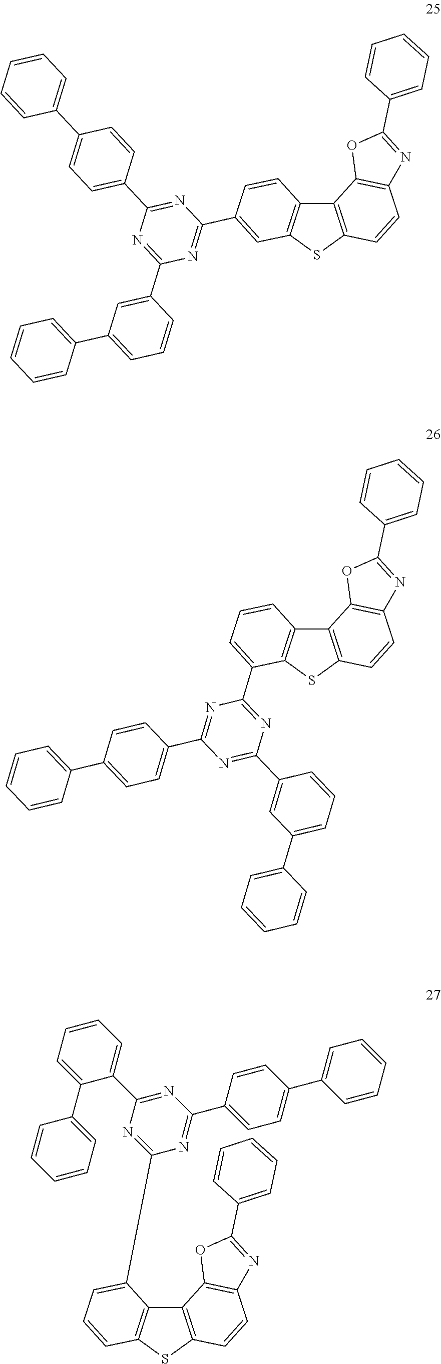 [SMILES: c1ccc(-c2ccc(-c3nc(-c4cccc(-c5ccccc5)c4)nc(-c4ccc5c(c4)sc4ccc6nc(-c7ccccc7)oc6c45)n3)cc2)cc1 ; c1ccc(-c2ccc(-c3nc(-c4cccc(-c5ccccc5)c4)nc(-c4cccc5c4sc4ccc6nc(-c7ccccc7)oc6c45)n3)cc2)cc1 ; c1ccc(-c2ccc(-c3nc(-c4ccccc4-c4ccccc4)nc(-c4cccc5sc6ccc7nc(-c8ccccc8)oc7c6c45)n3)cc2)cc1]

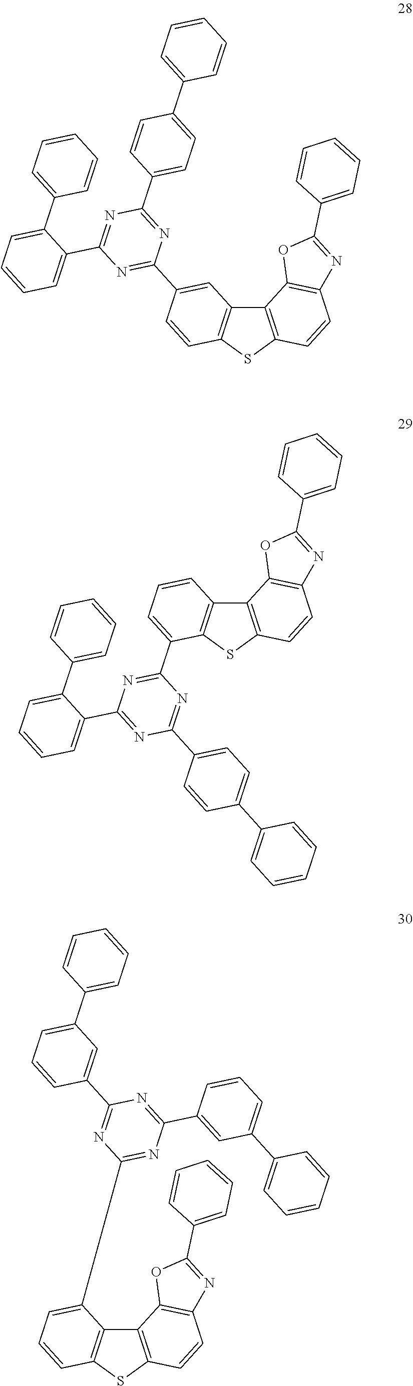 [SMILES: c1ccc(-c2ccc(-c3nc(-c4ccc5sc6ccc7nc(-c8ccccc8)oc7c6c5c4)nc(-c4ccccc4-c4ccccc4)n3)cc2)cc1 ; c1ccc(-c2ccc(-c3nc(-c4ccccc4-c4ccccc4)nc(-c4cccc5c4sc4ccc6nc(-c7ccccc7)oc6c45)n3)cc2)cc1 ; c1ccc(-c2cccc(-c3nc(-c4cccc(-c5ccccc5)c4)nc(-c4cccc5sc6ccc7nc(-c8ccccc8)oc7c6c45)n3)c2)cc1]

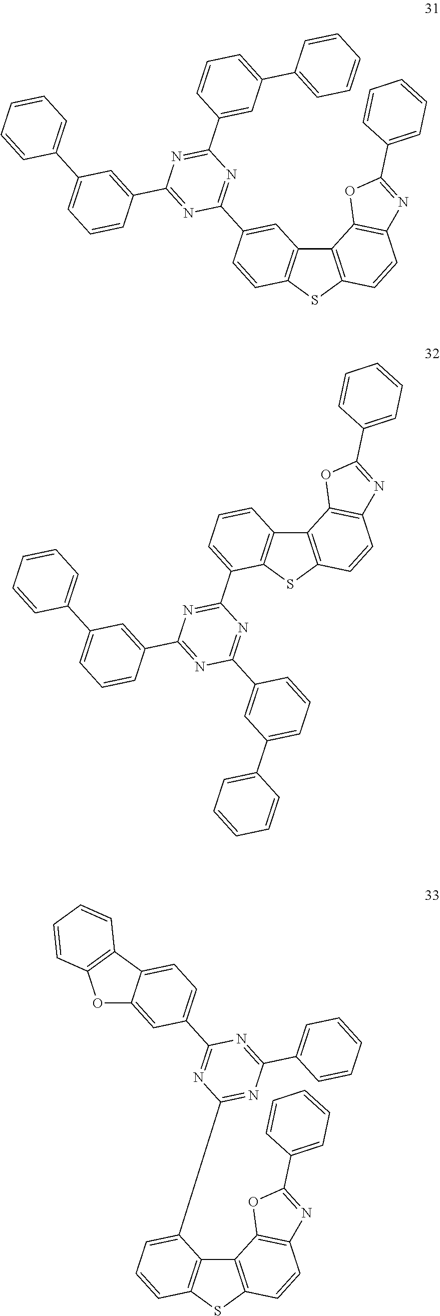 [SMILES: c1ccc(-c2cccc(-c3nc(-c4cccc(-c5ccccc5)c4)nc(-c4ccc5sc6ccc7nc(-c8ccccc8)oc7c6c5c4)n3)c2)cc1 ; c1ccc(-c2cccc(-c3nc(-c4cccc(-c5ccccc5)c4)nc(-c4cccc5c4sc4ccc6nc(-c7ccccc7)oc6c45)n3)c2)cc1 ; c1ccc(-c2nc(-c3ccc4c(c3)oc3ccccc34)nc(-c3cccc4sc5ccc6nc(-c7ccccc7)oc6c5c34)n2)cc1]

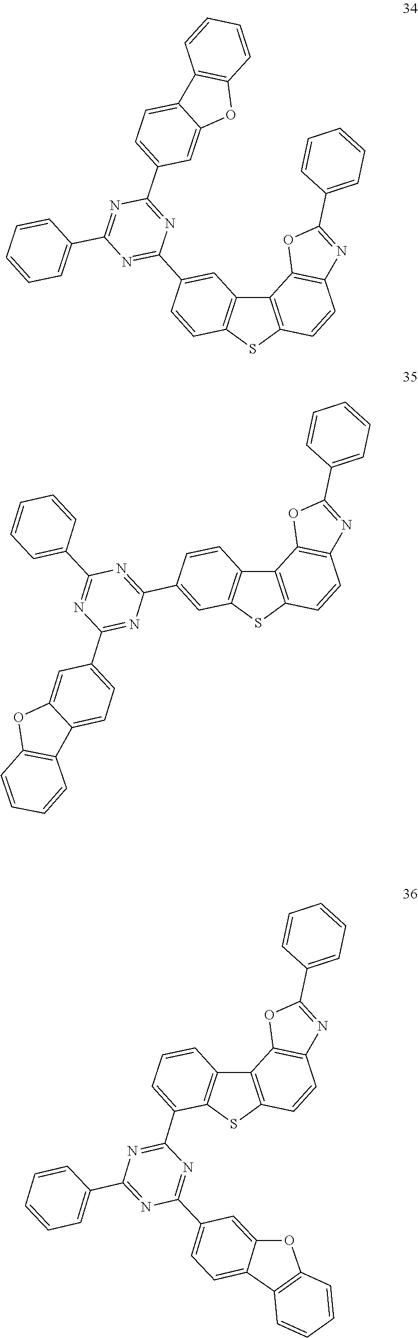 [SMILES: c1ccc(-c2nc(-c3ccc4c(c3)oc3ccccc34)nc(-c3ccc4sc5ccc6nc(-c7ccccc7)oc6c5c4c3)n2)cc1 ; c1ccc(-c2nc(-c3ccc4c(c3)oc3ccccc34)nc(-c3ccc4c(c3)sc3ccc5nc(-c6ccccc6)oc5c34)n2)cc1 ; c1ccc(-c2nc(-c3ccc4c(c3)oc3ccccc34)nc(-c3cccc4c3sc3ccc5nc(-c6ccccc6)oc5c34)n2)cc1]

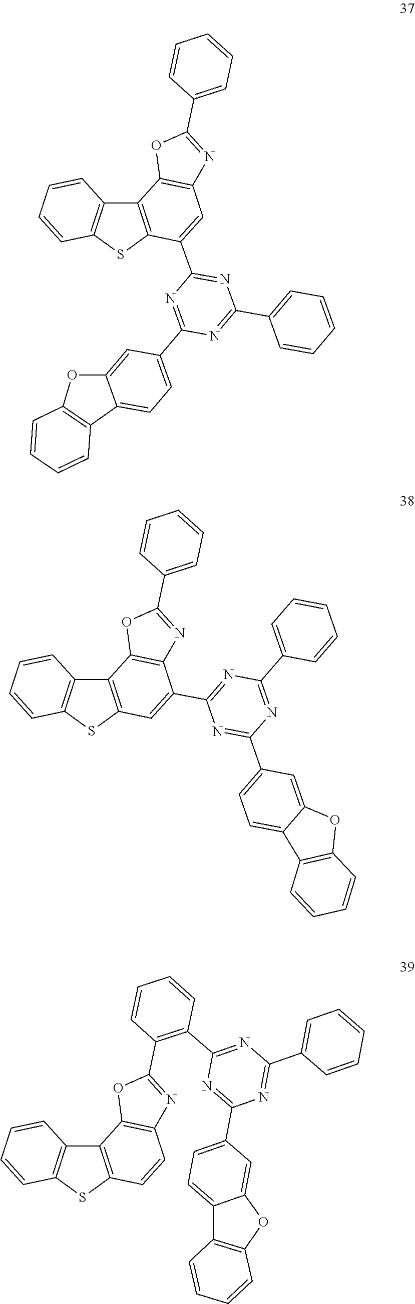 [SMILES: c1ccc(-c2nc(-c3ccc4c(c3)oc3ccccc34)nc(-c3cc4nc(-c5ccccc5)oc4c4c3sc3ccccc34)n2)cc1 ; c1ccc(-c2nc(-c3ccc4c(c3)oc3ccccc34)nc(-c3cc4sc5ccccc5c4c4oc(-c5ccccc5)nc34)n2)cc1 ; c1ccc(-c2nc(-c3ccc4c(c3)oc3ccccc34)nc(-c3ccccc3-c3nc4ccc5sc6ccccc6c5c4o3)n2)cc1]

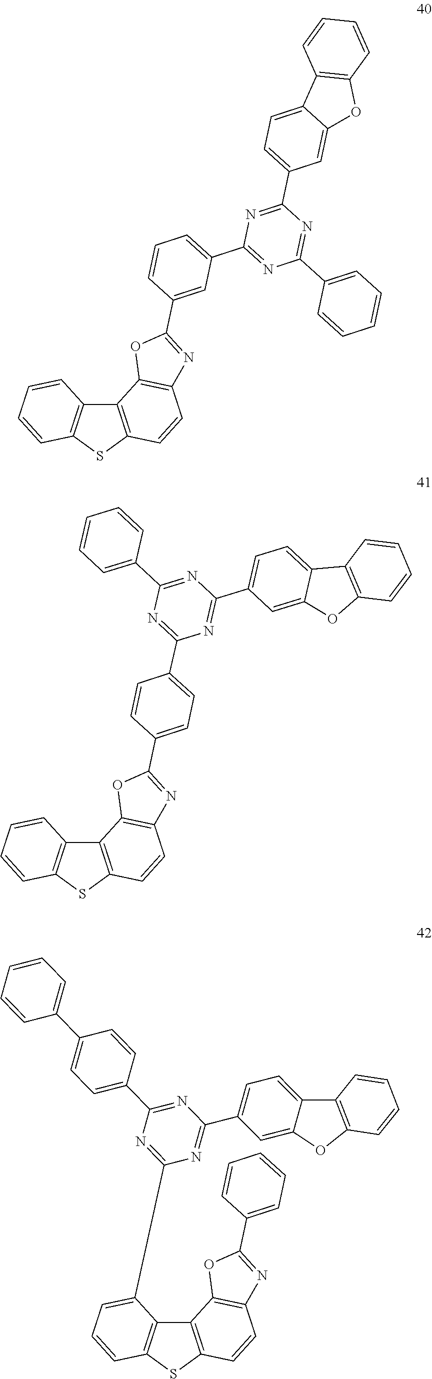 [SMILES: c1ccc(-c2nc(-c3cccc(-c4nc5ccc6sc7ccccc7c6c5o4)c3)nc(-c3ccc4c(c3)oc3ccccc34)n2)cc1 ; c1ccc(-c2nc(-c3ccc(-c4nc5ccc6sc7ccccc7c6c5o4)cc3)nc(-c3ccc4c(c3)oc3ccccc34)n2)cc1 ; c1ccc(-c2ccc(-c3nc(-c4ccc5c(c4)oc4ccccc45)nc(-c4cccc5sc6ccc7nc(-c8ccccc8)oc7c6c45)n3)cc2)cc1]

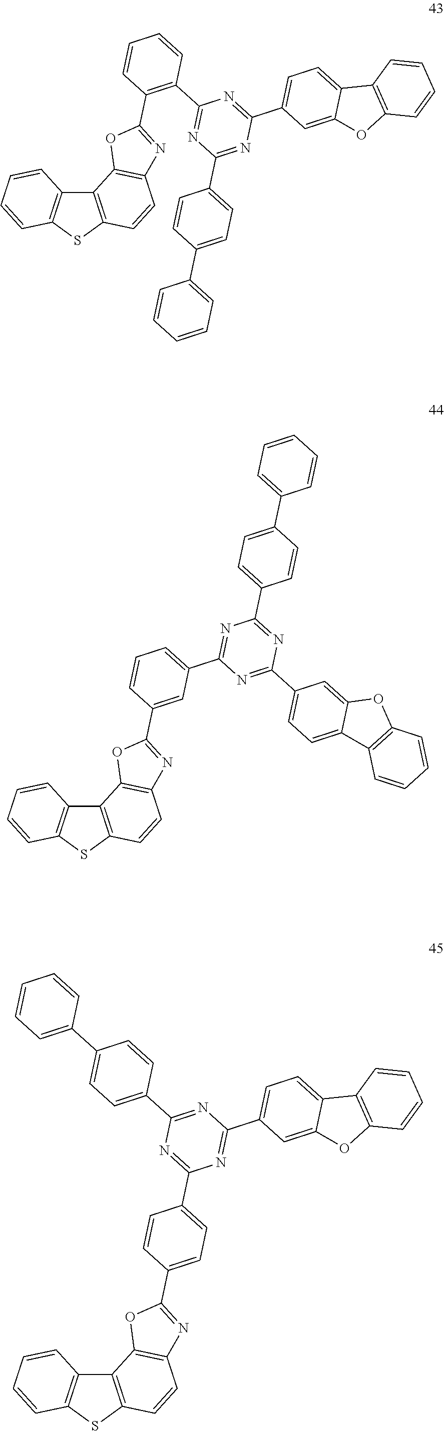 [SMILES: c1ccc(-c2ccc(-c3nc(-c4ccc5c(c4)oc4ccccc45)nc(-c4ccccc4-c4nc5ccc6sc7ccccc7c6c5o4)n3)cc2)cc1 ; c1ccc(-c2ccc(-c3nc(-c4cccc(-c5nc6ccc7sc8ccccc8c7c6o5)c4)nc(-c4ccc5c(c4)oc4ccccc45)n3)cc2)cc1 ; c1ccc(-c2ccc(-c3nc(-c4ccc(-c5nc6ccc7sc8ccccc8c7c6o5)cc4)nc(-c4ccc5c(c4)oc4ccccc45)n3)cc2)cc1]

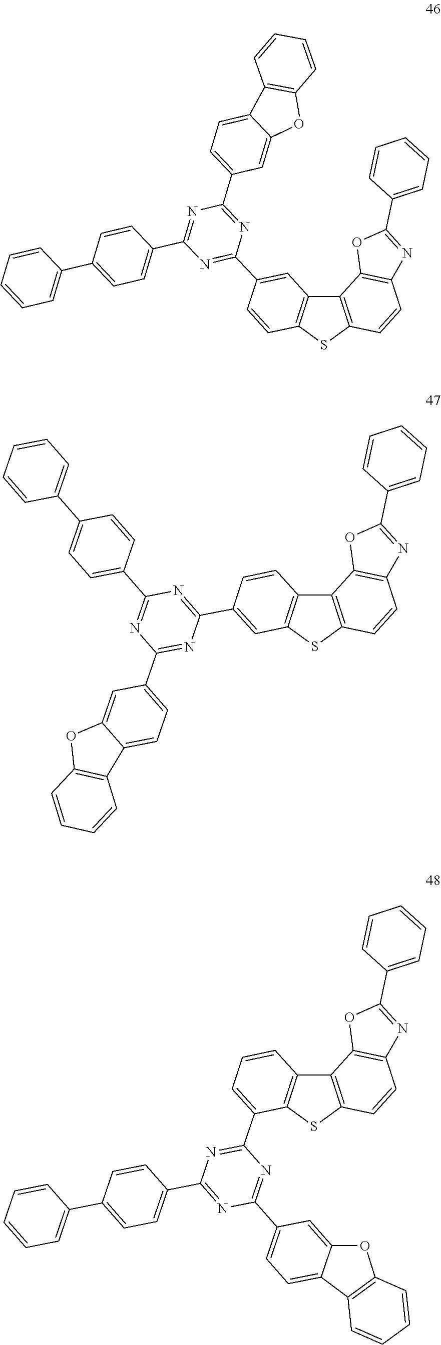 [SMILES: c1ccc(-c2ccc(-c3nc(-c4ccc5c(c4)oc4ccccc45)nc(-c4ccc5sc6ccc7nc(-c8ccccc8)oc7c6c5c4)n3)cc2)cc1 ; c1ccc(-c2ccc(-c3nc(-c4ccc5c(c4)oc4ccccc45)nc(-c4ccc5c(c4)sc4ccc6nc(-c7ccccc7)oc6c45)n3)cc2)cc1 ; c1ccc(-c2ccc(-c3nc(-c4ccc5c(c4)oc4ccccc45)nc(-c4cccc5c4sc4ccc6nc(-c7ccccc7)oc6c45)n3)cc2)cc1]

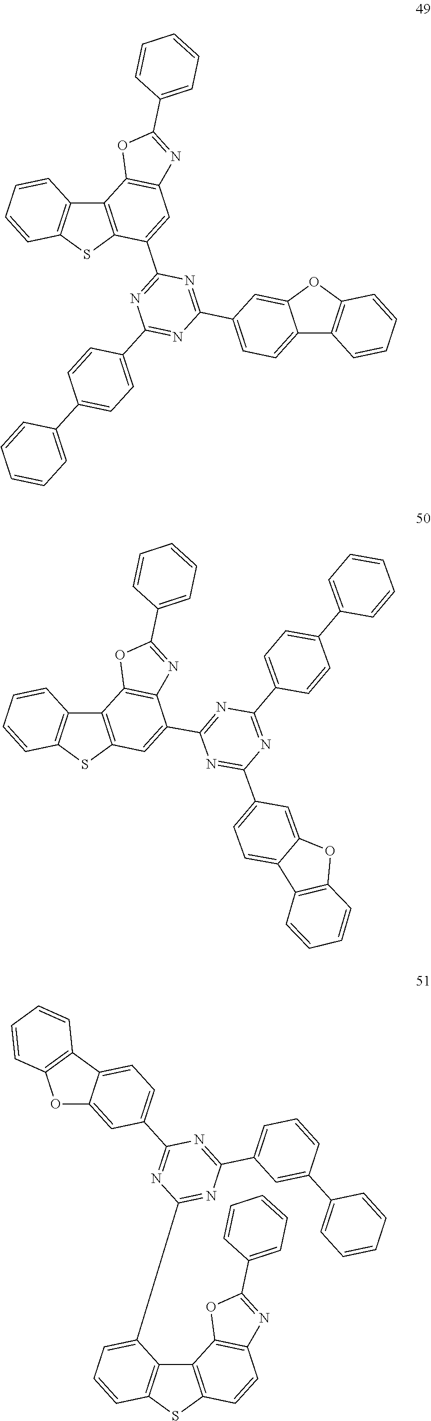 [SMILES: c1ccc(-c2ccc(-c3nc(-c4ccc5c(c4)oc4ccccc45)nc(-c4cc5nc(-c6ccccc6)oc5c5c4sc4ccccc45)n3)cc2)cc1 ; c1ccc(-c2ccc(-c3nc(-c4ccc5c(c4)oc4ccccc45)nc(-c4cc5sc6ccccc6c5c5oc(-c6ccccc6)nc45)n3)cc2)cc1 ; c1ccc(-c2cccc(-c3nc(-c4ccc5c(c4)oc4ccccc45)nc(-c4cccc5sc6ccc7nc(-c8ccccc8)oc7c6c45)n3)c2)cc1]

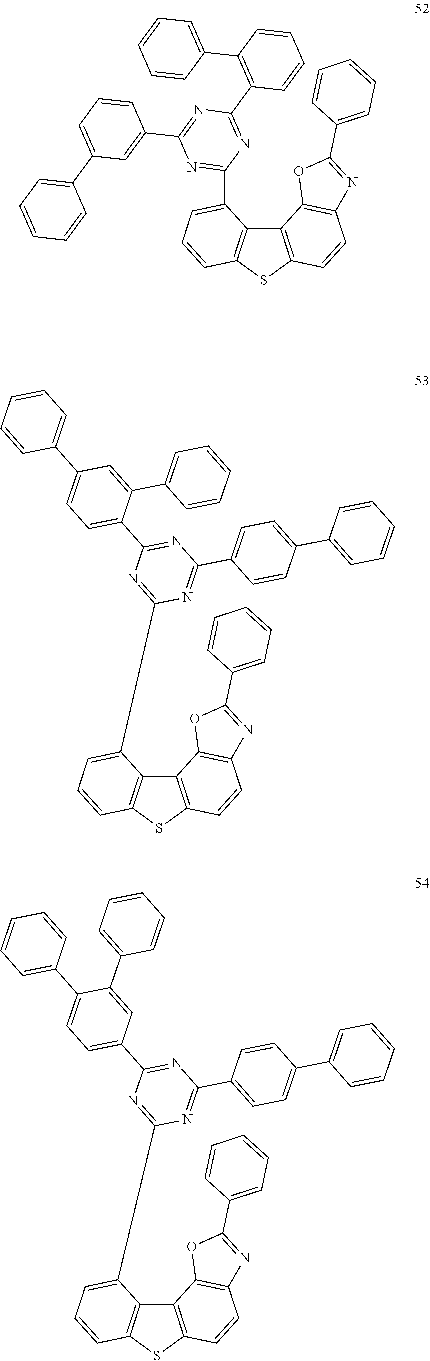 [SMILES: c1ccc(-c2cccc(-c3nc(-c4ccccc4-c4ccccc4)nc(-c4cccc5sc6ccc7nc(-c8ccccc8)oc7c6c45)n3)c2)cc1 ; c1ccc(-c2ccc(-c3nc(-c4ccc(-c5ccccc5)cc4-c4ccccc4)nc(-c4cccc5sc6ccc7nc(-c8ccccc8)oc7c6c45)n3)cc2)cc1 ; c1ccc(-c2ccc(-c3nc(-c4ccc(-c5ccccc5)c(-c5ccccc5)c4)nc(-c4cccc5sc6ccc7nc(-c8ccccc8)oc7c6c45)n3)cc2)cc1]

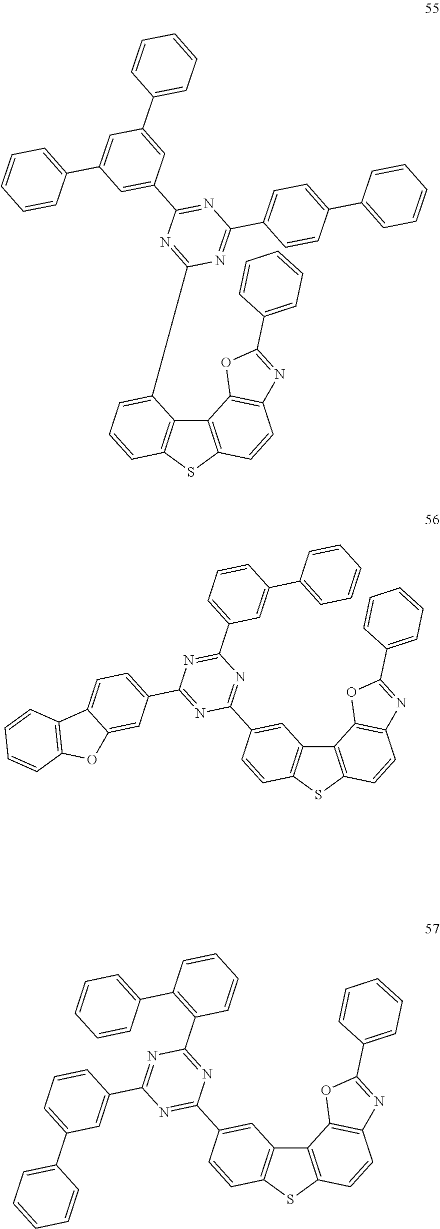 [SMILES: c1ccc(-c2ccc(-c3nc(-c4cc(-c5ccccc5)cc(-c5ccccc5)c4)nc(-c4cccc5sc6ccc7nc(-c8ccccc8)oc7c6c45)n3)cc2)cc1 ; c1ccc(-c2cccc(-c3nc(-c4ccc5c(c4)oc4ccccc45)nc(-c4ccc5sc6ccc7nc(-c8ccccc8)oc7c6c5c4)n3)c2)cc1 ; c1ccc(-c2cccc(-c3nc(-c4ccc5sc6ccc7nc(-c8ccccc8)oc7c6c5c4)nc(-c4ccccc4-c4ccccc4)n3)c2)cc1]

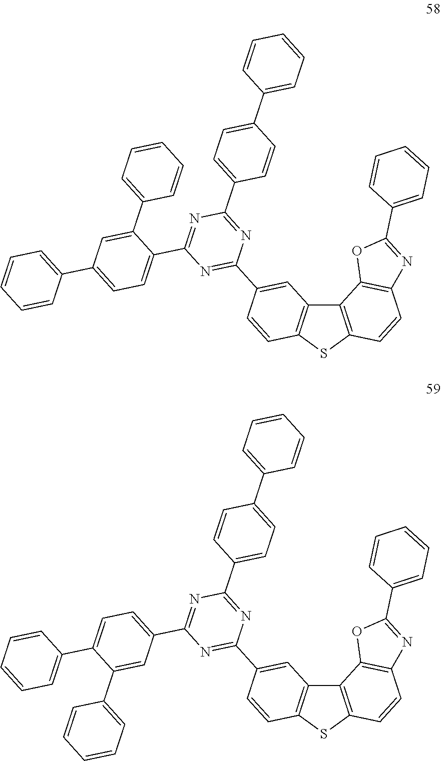 [SMILES: c1ccc(-c2ccc(-c3nc(-c4ccc5sc6ccc7nc(-c8ccccc8)oc7c6c5c4)nc(-c4ccc(-c5ccccc5)cc4-c4ccccc4)n3)cc2)cc1 ; c1ccc(-c2ccc(-c3nc(-c4ccc(-c5ccccc5)c(-c5ccccc5)c4)nc(-c4ccc5sc6ccc7nc(-c8ccccc8)oc7c6c5c4)n3)cc2)cc1]

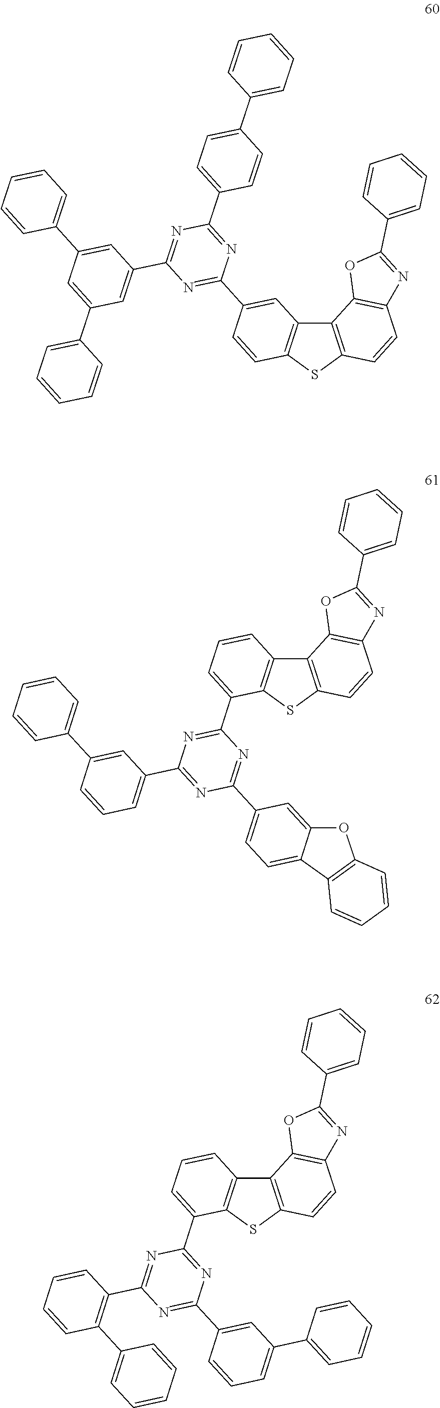 [SMILES: c1ccc(-c2ccc(-c3nc(-c4cc(-c5ccccc5)cc(-c5ccccc5)c4)nc(-c4ccc5sc6ccc7nc(-c8ccccc8)oc7c6c5c4)n3)cc2)cc1 ; c1ccc(-c2cccc(-c3nc(-c4ccc5c(c4)oc4ccccc45)nc(-c4cccc5c4sc4ccc6nc(-c7ccccc7)oc6c45)n3)c2)cc1 ; c1ccc(-c2cccc(-c3nc(-c4ccccc4-c4ccccc4)nc(-c4cccc5c4sc4ccc6nc(-c7ccccc7)oc6c45)n3)c2)cc1]

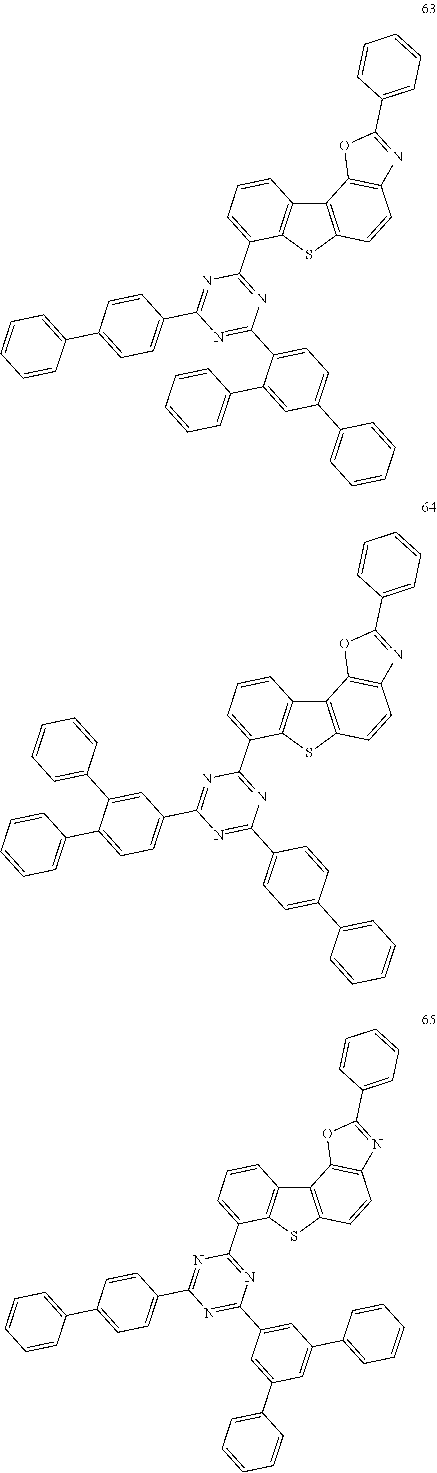 [SMILES: c1ccc(-c2ccc(-c3nc(-c4ccc(-c5ccccc5)cc4-c4ccccc4)nc(-c4cccc5c4sc4ccc6nc(-c7ccccc7)oc6c45)n3)cc2)cc1 ; c1ccc(-c2ccc(-c3nc(-c4ccc(-c5ccccc5)c(-c5ccccc5)c4)nc(-c4cccc5c4sc4ccc6nc(-c7ccccc7)oc6c45)n3)cc2)cc1 ; c1ccc(-c2ccc(-c3nc(-c4cc(-c5ccccc5)cc(-c5ccccc5)c4)nc(-c4cccc5c4sc4ccc6nc(-c7ccccc7)oc6c45)n3)cc2)cc1]

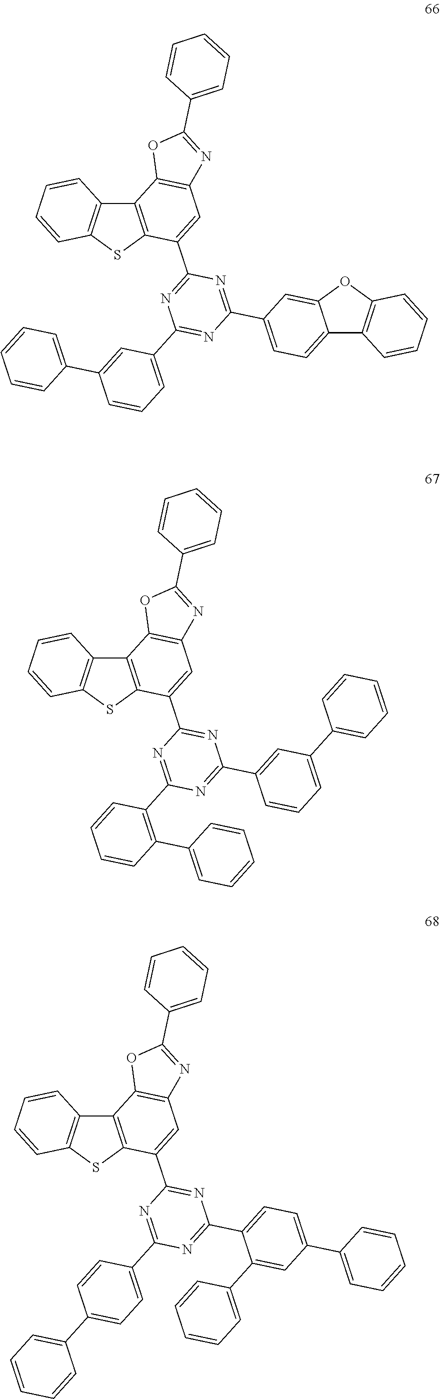 [SMILES: c1ccc(-c2cccc(-c3nc(-c4ccc5c(c4)oc4ccccc45)nc(-c4cc5nc(-c6ccccc6)oc5c5c4sc4ccccc45)n3)c2)cc1 ; c1ccc(-c2cccc(-c3nc(-c4ccccc4-c4ccccc4)nc(-c4cc5nc(-c6ccccc6)oc5c5c4sc4ccccc45)n3)c2)cc1 ; c1ccc(-c2ccc(-c3nc(-c4ccc(-c5ccccc5)cc4-c4ccccc4)nc(-c4cc5nc(-c6ccccc6)oc5c5c4sc4ccccc45)n3)cc2)cc1]

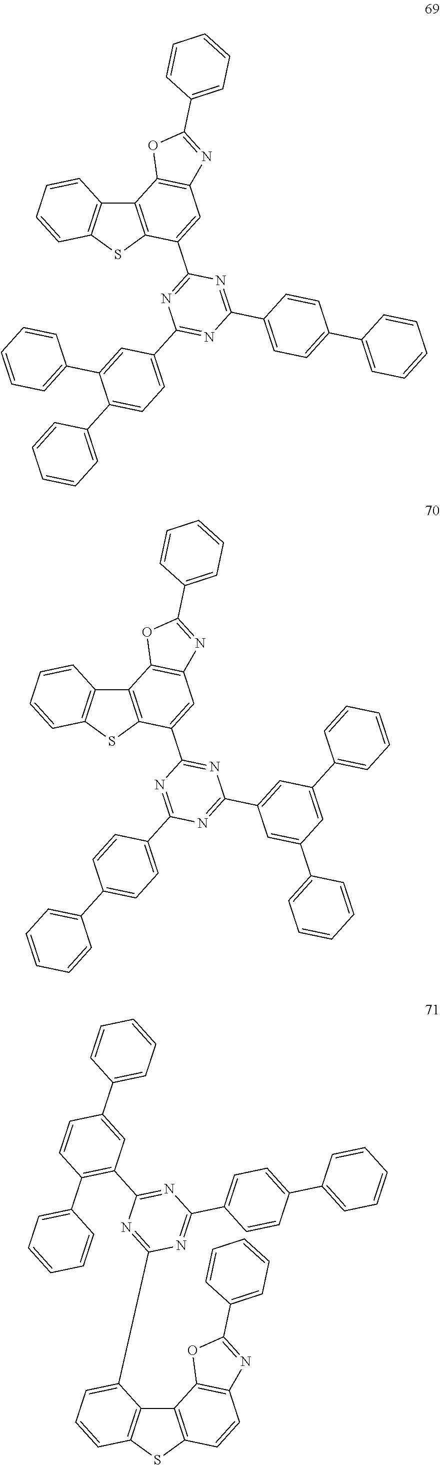 [SMILES: c1ccc(-c2ccc(-c3nc(-c4ccc(-c5ccccc5)c(-c5ccccc5)c4)nc(-c4cc5nc(-c6ccccc6)oc5c5c4sc4ccccc45)n3)cc2)cc1 ; c1ccc(-c2ccc(-c3nc(-c4cc(-c5ccccc5)cc(-c5ccccc5)c4)nc(-c4cc5nc(-c6ccccc6)oc5c5c4sc4ccccc45)n3)cc2)cc1 ; c1ccc(-c2ccc(-c3nc(-c4cc(-c5ccccc5)ccc4-c4ccccc4)nc(-c4cccc5sc6ccc7nc(-c8ccccc8)oc7c6c45)n3)cc2)cc1]

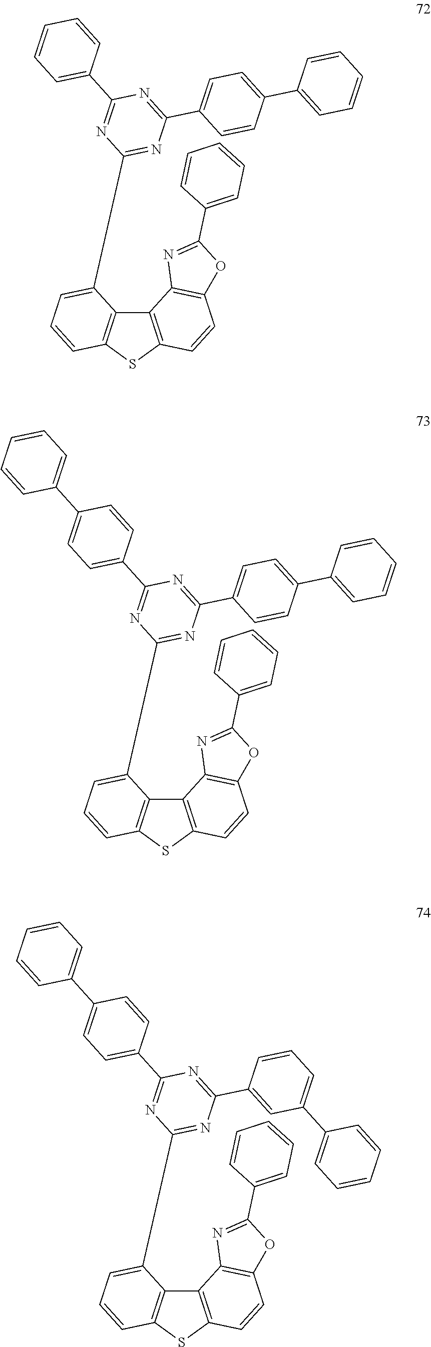 [SMILES: c1ccc(-c2ccc(-c3nc(-c4ccccc4)nc(-c4cccc5sc6ccc7oc(-c8ccccc8)nc7c6c45)n3)cc2)cc1 ; c1ccc(-c2ccc(-c3nc(-c4ccc(-c5ccccc5)cc4)nc(-c4cccc5sc6ccc7oc(-c8ccccc8)nc7c6c45)n3)cc2)cc1 ; c1ccc(-c2ccc(-c3nc(-c4cccc(-c5ccccc5)c4)nc(-c4cccc5sc6ccc7oc(-c8ccccc8)nc7c6c45)n3)cc2)cc1]

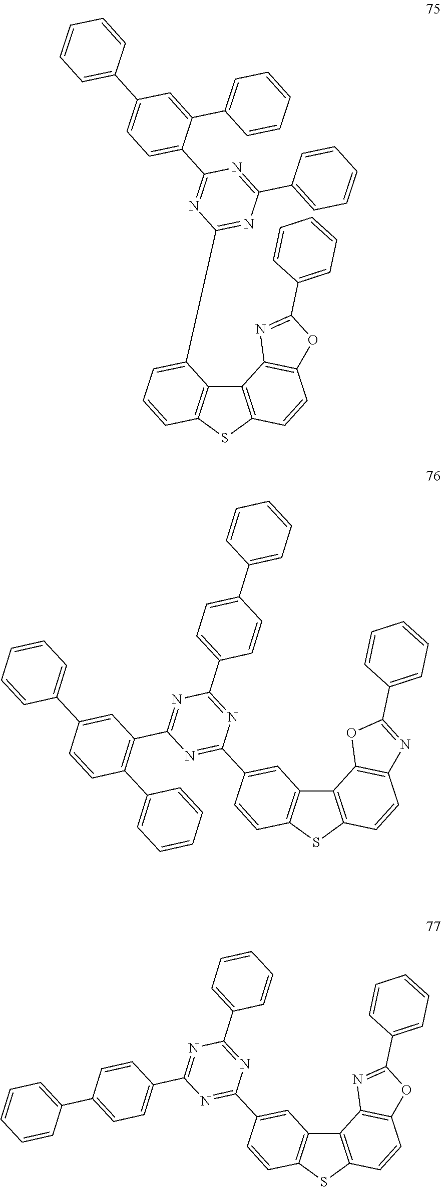 [SMILES: c1ccc(-c2ccc(-c3nc(-c4ccccc4)nc(-c4cccc5sc6ccc7oc(-c8ccccc8)nc7c6c45)n3)c(-c3ccccc3)c2)cc1 ; c1ccc(-c2ccc(-c3nc(-c4ccc5sc6ccc7nc(-c8ccccc8)oc7c6c5c4)nc(-c4cc(-c5ccccc5)ccc4-c4ccccc4)n3)cc2)cc1 ; c1ccc(-c2ccc(-c3nc(-c4ccccc4)nc(-c4ccc5sc6ccc7oc(-c8ccccc8)nc7c6c5c4)n3)cc2)cc1]

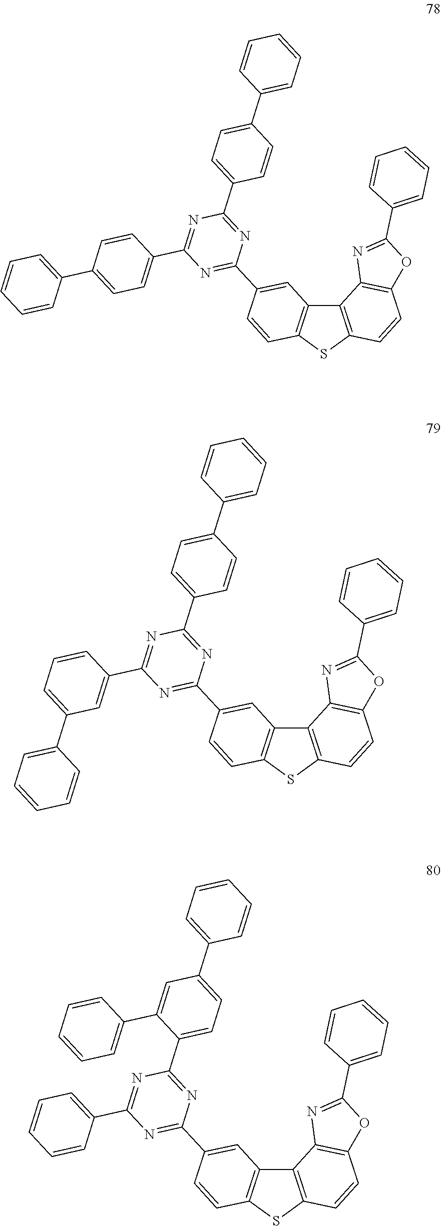 [SMILES: c1ccc(-c2ccc(-c3nc(-c4ccc(-c5ccccc5)cc4)nc(-c4ccc5sc6ccc7oc(-c8ccccc8)nc7c6c5c4)n3)cc2)cc1 ; c1ccc(-c2ccc(-c3nc(-c4cccc(-c5ccccc5)c4)nc(-c4ccc5sc6ccc7oc(-c8ccccc8)nc7c6c5c4)n3)cc2)cc1 ; c1ccc(-c2ccc(-c3nc(-c4ccccc4)nc(-c4ccc5sc6ccc7oc(-c8ccccc8)nc7c6c5c4)n3)c(-c3ccccc3)c2)cc1]

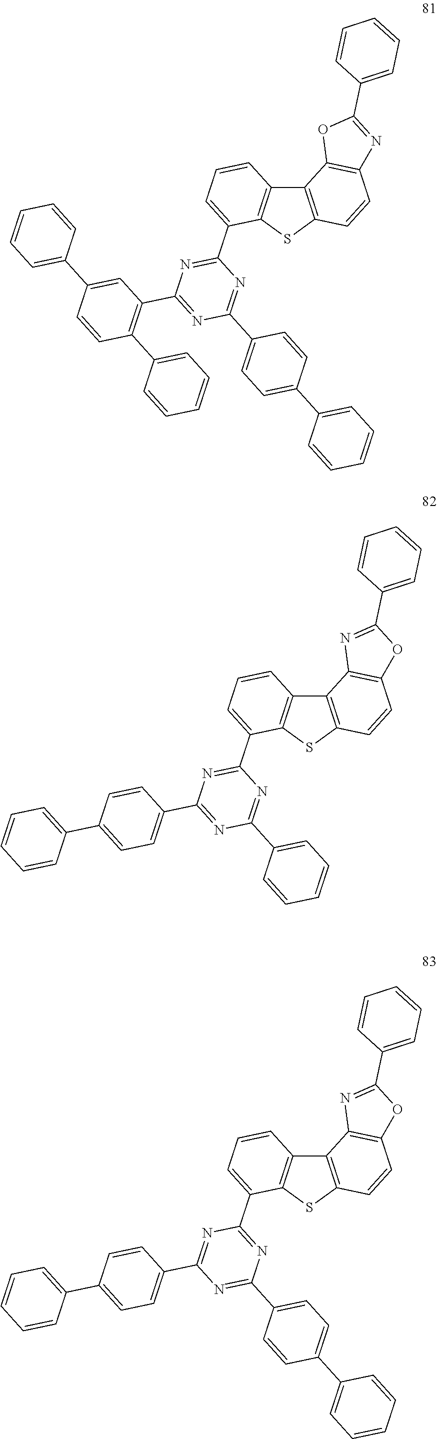 [SMILES: c1ccc(-c2ccc(-c3nc(-c4cc(-c5ccccc5)ccc4-c4ccccc4)nc(-c4cccc5c4sc4ccc6nc(-c7ccccc7)oc6c45)n3)cc2)cc1 ; c1ccc(-c2ccc(-c3nc(-c4ccccc4)nc(-c4cccc5c4sc4ccc6oc(-c7ccccc7)nc6c45)n3)cc2)cc1 ; c1ccc(-c2ccc(-c3nc(-c4ccc(-c5ccccc5)cc4)nc(-c4cccc5c4sc4ccc6oc(-c7ccccc7)nc6c45)n3)cc2)cc1]

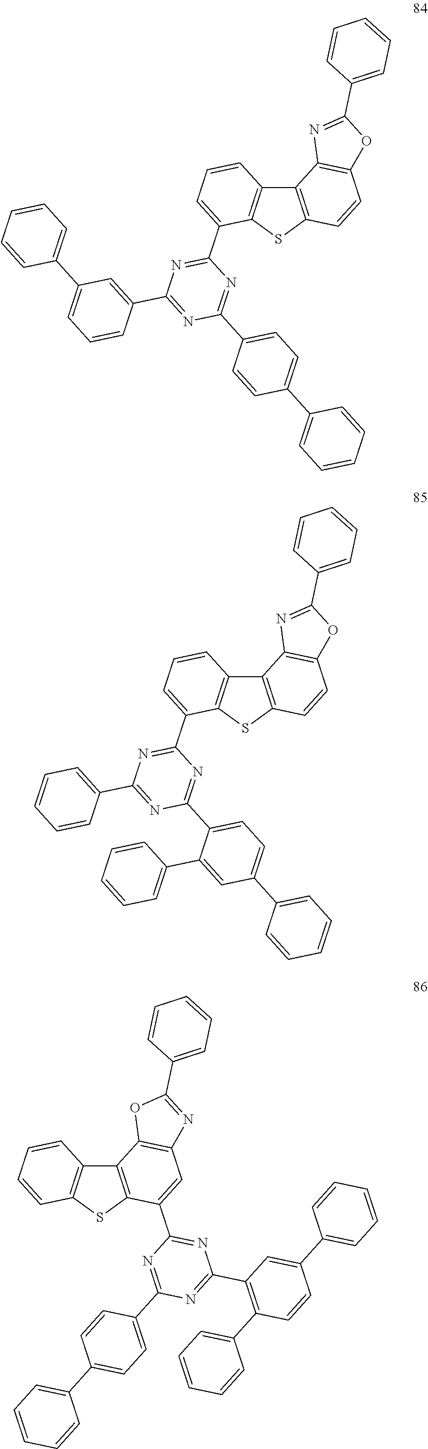

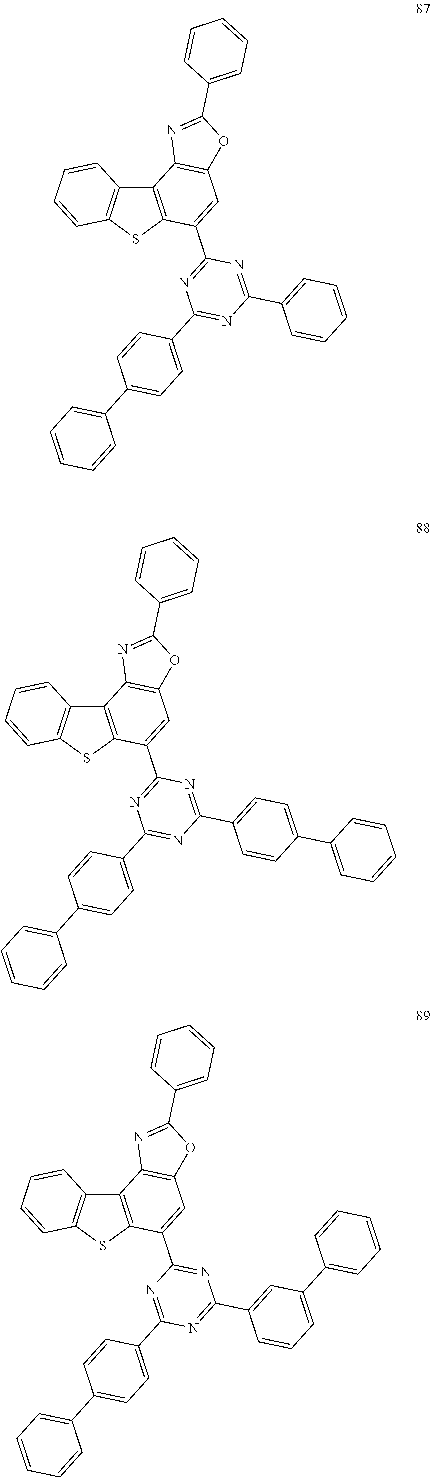 [SMILES: c1ccc(-c2ccc(-c3nc(-c4ccccc4)nc(-c4cc5oc(-c6ccccc6)nc5c5c4sc4ccccc45)n3)cc2)cc1 ; c1ccc(-c2ccc(-c3nc(-c4ccc(-c5ccccc5)cc4)nc(-c4cc5oc(-c6ccccc6)nc5c5c4sc4ccccc45)n3)cc2)cc1 ; c1ccc(-c2ccc(-c3nc(-c4cccc(-c5ccccc5)c4)nc(-c4cc5oc(-c6ccccc6)nc5c5c4sc4ccccc45)n3)cc2)cc1]

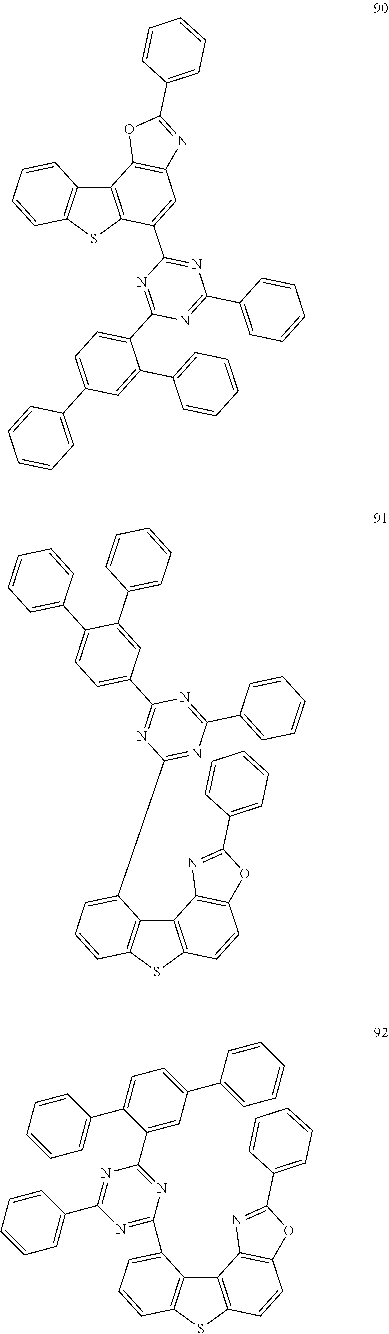 [SMILES: c1ccc(-c2ccc(-c3nc(-c4ccccc4)nc(-c4cc5nc(-c6ccccc6)oc5c5c4sc4ccccc45)n3)c(-c3ccccc3)c2)cc1 ; c1ccc(-c2nc(-c3ccc(-c4ccccc4)c(-c4ccccc4)c3)nc(-c3cccc4sc5ccc6oc(-c7ccccc7)nc6c5c34)n2)cc1 ; c1ccc(-c2ccc(-c3ccccc3)c(-c3nc(-c4ccccc4)nc(-c4cccc5sc6ccc7oc(-c8ccccc8)nc7c6c45)n3)c2)cc1]

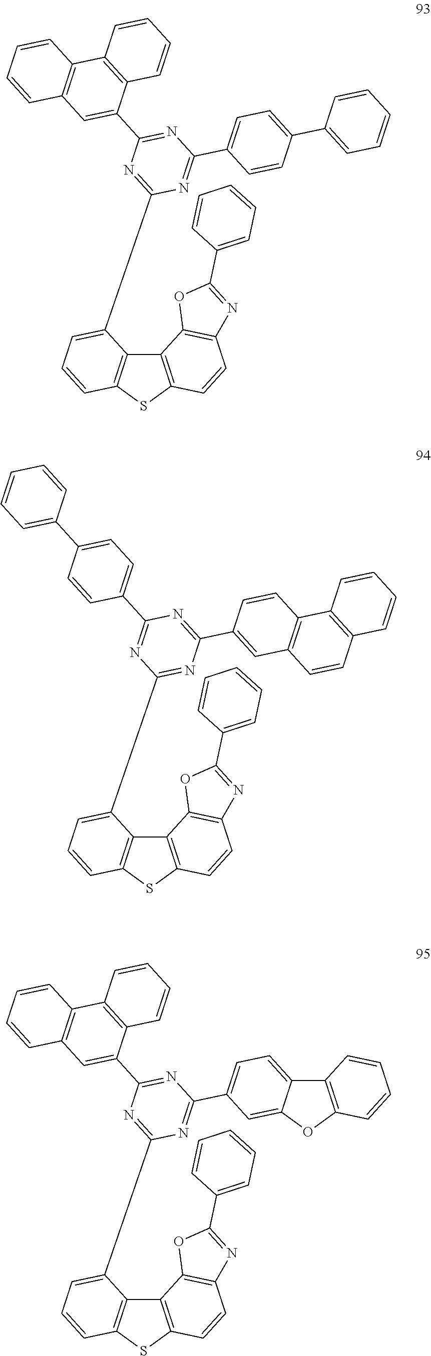 [SMILES: c1ccc(-c2ccc(-c3nc(-c4cc5ccccc5c5ccccc45)nc(-c4cccc5sc6ccc7nc(-c8ccccc8)oc7c6c45)n3)cc2)cc1 ; c1ccc(-c2ccc(-c3nc(-c4ccc5c(ccc6ccccc65)c4)nc(-c4cccc5sc6ccc7nc(-c8ccccc8)oc7c6c45)n3)cc2)cc1 ; c1ccc(-c2nc3ccc4sc5cccc(-c6nc(-c7ccc8c(c7)oc7ccccc78)nc(-c7cc8ccccc8c8ccccc78)n6)c5c4c3o2)cc1]

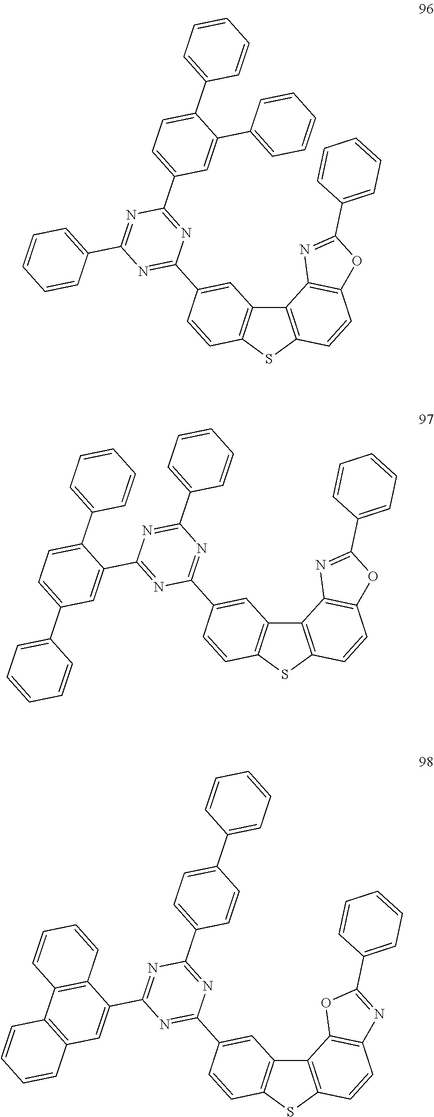 [SMILES: c1ccc(-c2nc(-c3ccc(-c4ccccc4)c(-c4ccccc4)c3)nc(-c3ccc4sc5ccc6oc(-c7ccccc7)nc6c5c4c3)n2)cc1 ; c1ccc(-c2ccc(-c3ccccc3)c(-c3nc(-c4ccccc4)nc(-c4ccc5sc6ccc7oc(-c8ccccc8)nc7c6c5c4)n3)c2)cc1 ; c1ccc(-c2ccc(-c3nc(-c4ccc5sc6ccc7nc(-c8ccccc8)oc7c6c5c4)nc(-c4cc5ccccc5c5ccccc45)n3)cc2)cc1]

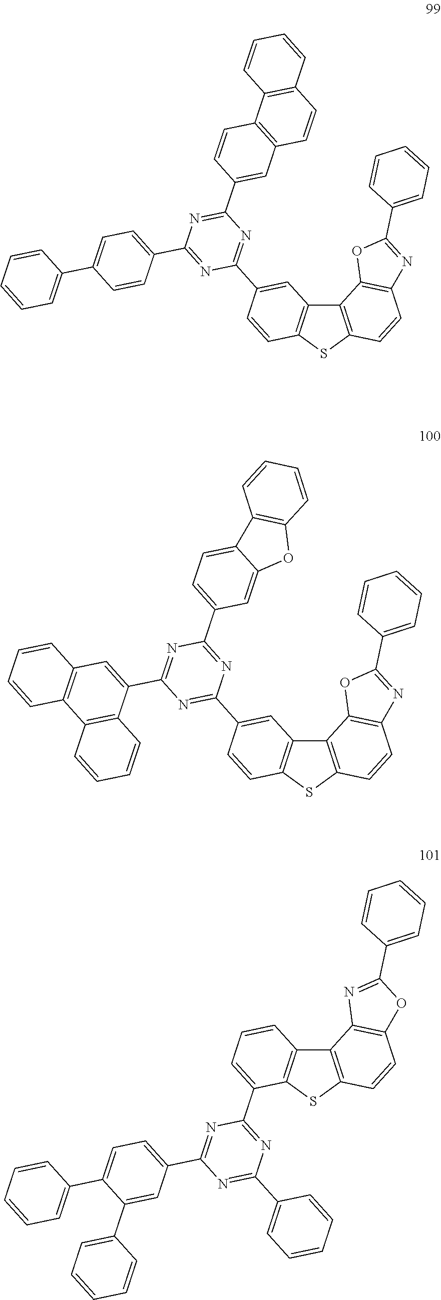 [SMILES: c1ccc(-c2ccc(-c3nc(-c4ccc5c(ccc6ccccc65)c4)nc(-c4ccc5sc6ccc7nc(-c8ccccc8)oc7c6c5c4)n3)cc2)cc1 ; c1ccc(-c2nc3ccc4sc5ccc(-c6nc(-c7ccc8c(c7)oc7ccccc78)nc(-c7cc8ccccc8c8ccccc78)n6)cc5c4c3o2)cc1 ; c1ccc(-c2nc(-c3ccc(-c4ccccc4)c(-c4ccccc4)c3)nc(-c3cccc4c3sc3ccc5oc(-c6ccccc6)nc5c34)n2)cc1]

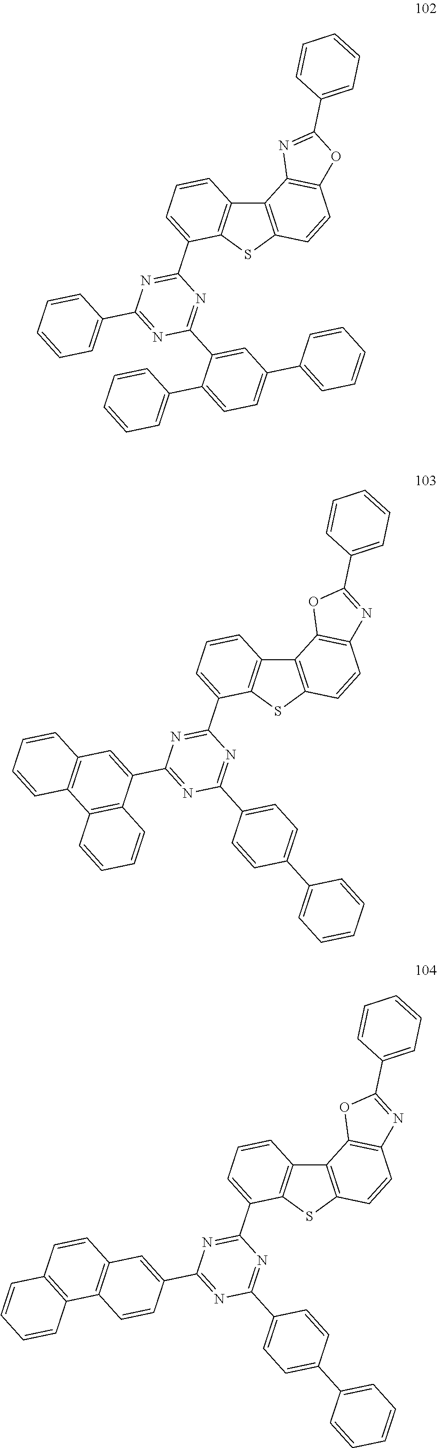 [SMILES: c1ccc(-c2ccc(-c3ccccc3)c(-c3nc(-c4ccccc4)nc(-c4cccc5c4sc4ccc6oc(-c7ccccc7)nc6c45)n3)c2)cc1 ; c1ccc(-c2ccc(-c3nc(-c4cc5ccccc5c5ccccc45)nc(-c4cccc5c4sc4ccc6nc(-c7ccccc7)oc6c45)n3)cc2)cc1 ; c1ccc(-c2ccc(-c3nc(-c4ccc5c(ccc6ccccc65)c4)nc(-c4cccc5c4sc4ccc6nc(-c7ccccc7)oc6c45)n3)cc2)cc1]

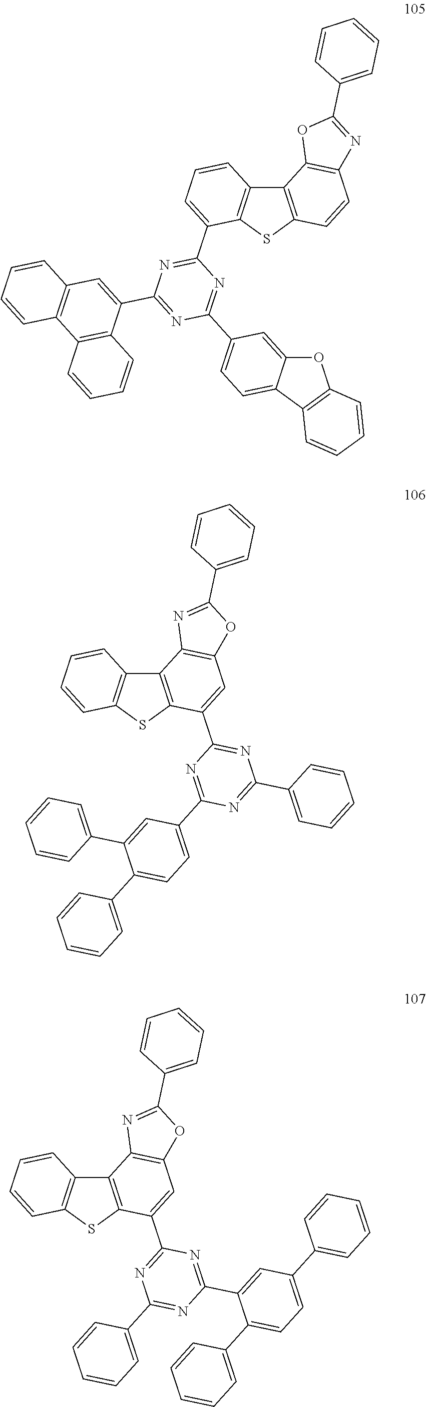 [SMILES: c1ccc(-c2nc3ccc4sc5c(-c6nc(-c7ccc8c(c7)oc7ccccc78)nc(-c7cc8ccccc8c8ccccc78)n6)cccc5c4c3o2)cc1 ; c1ccc(-c2nc(-c3ccc(-c4ccccc4)c(-c4ccccc4)c3)nc(-c3cc4oc(-c5ccccc5)nc4c4c3sc3ccccc34)n2)cc1 ; c1ccc(-c2ccc(-c3ccccc3)c(-c3nc(-c4ccccc4)nc(-c4cc5oc(-c6ccccc6)nc5c5c4sc4ccccc45)n3)c2)cc1]

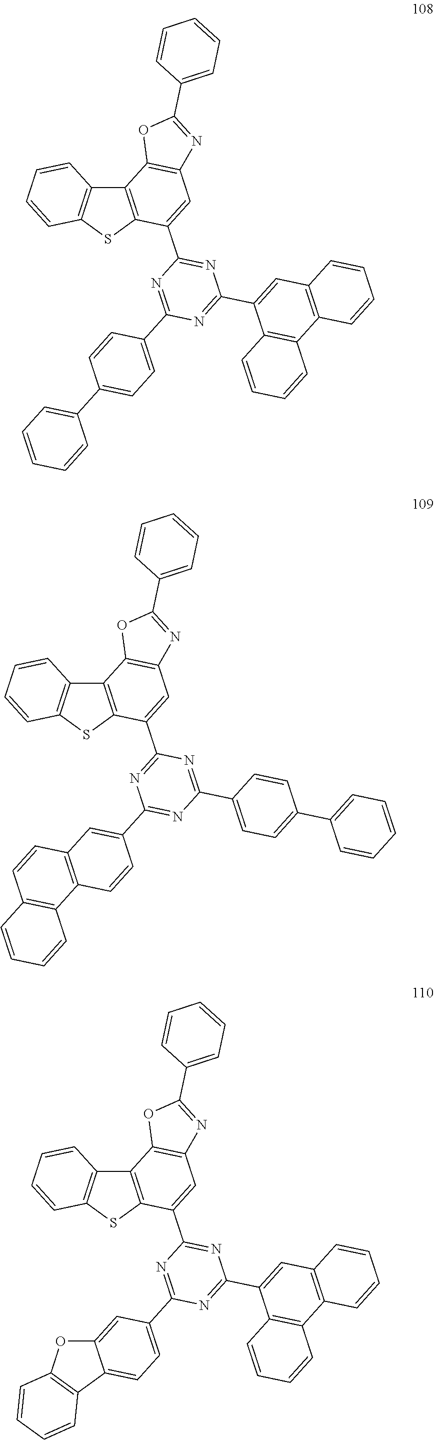 [SMILES: c1ccc(-c2ccc(-c3nc(-c4cc5ccccc5c5ccccc45)nc(-c4cc5nc(-c6ccccc6)oc5c5c4sc4ccccc45)n3)cc2)cc1 ; c1ccc(-c2ccc(-c3nc(-c4ccc5c(ccc6ccccc65)c4)nc(-c4cc5nc(-c6ccccc6)oc5c5c4sc4ccccc45)n3)cc2)cc1 ; c1ccc(-c2nc3cc(-c4nc(-c5ccc6c(c5)oc5ccccc56)nc(-c5cc6ccccc6c6ccccc56)n4)c4sc5ccccc5c4c3o2)cc1]

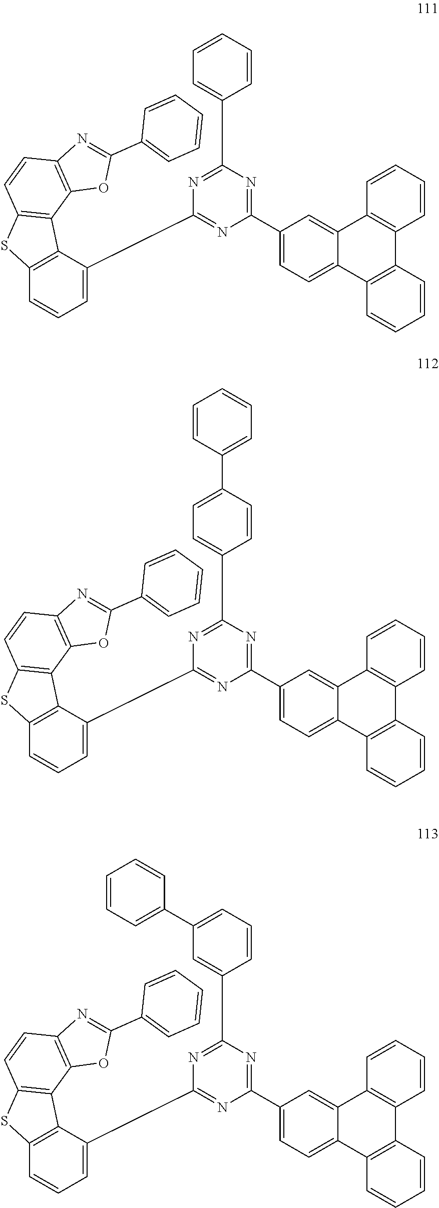 [SMILES: c1ccc(-c2nc(-c3ccc4c5ccccc5c5ccccc5c4c3)nc(-c3cccc4sc5ccc6nc(-c7ccccc7)oc6c5c34)n2)cc1 ; c1ccc(-c2ccc(-c3nc(-c4ccc5c6ccccc6c6ccccc6c5c4)nc(-c4cccc5sc6ccc7nc(-c8ccccc8)oc7c6c45)n3)cc2)cc1 ; c1ccc(-c2cccc(-c3nc(-c4ccc5c6ccccc6c6ccccc6c5c4)nc(-c4cccc5sc6ccc7nc(-c8ccccc8)oc7c6c45)n3)c2)cc1]

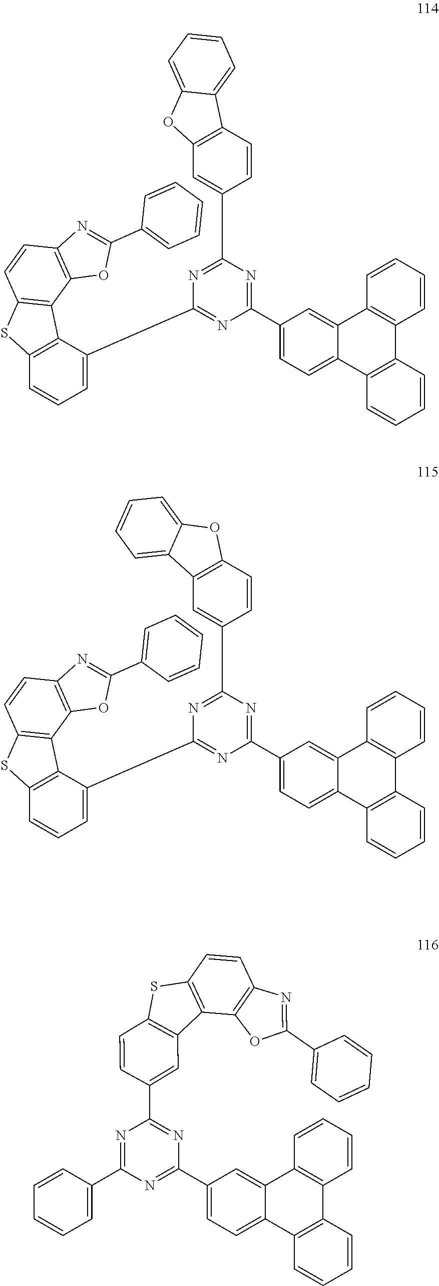 [SMILES: c1ccc(-c2nc3ccc4sc5cccc(-c6nc(-c7ccc8c(c7)oc7ccccc78)nc(-c7ccc8c9ccccc9c9ccccc9c8c7)n6)c5c4c3o2)cc1 ; c1ccc(-c2nc3ccc4sc5cccc(-c6nc(-c7ccc8oc9ccccc9c8c7)nc(-c7ccc8c9ccccc9c9ccccc9c8c7)n6)c5c4c3o2)cc1 ; c1ccc(-c2nc(-c3ccc4c5ccccc5c5ccccc5c4c3)nc(-c3ccc4sc5ccc6nc(-c7ccccc7)oc6c5c4c3)n2)cc1]

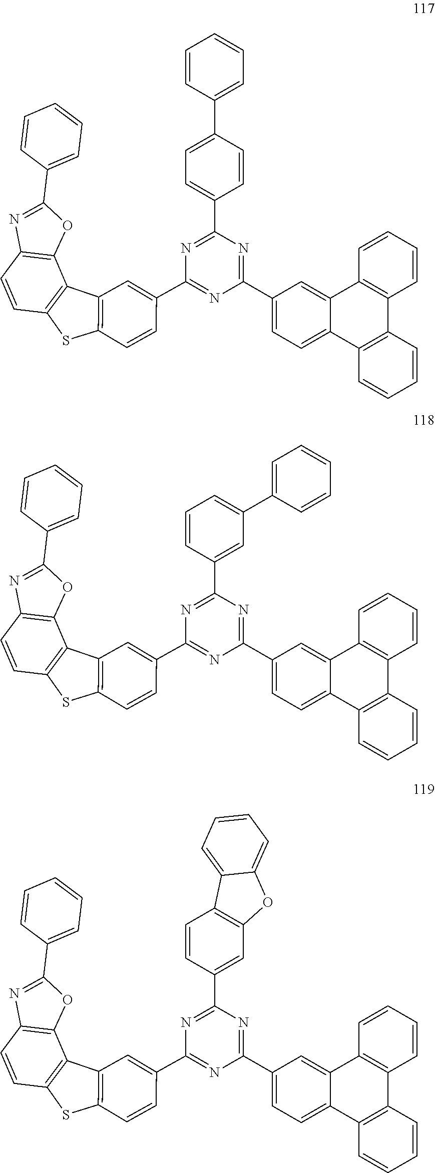 [SMILES: c1ccc(-c2ccc(-c3nc(-c4ccc5c6ccccc6c6ccccc6c5c4)nc(-c4ccc5sc6ccc7nc(-c8ccccc8)oc7c6c5c4)n3)cc2)cc1 ; c1ccc(-c2cccc(-c3nc(-c4ccc5c6ccccc6c6ccccc6c5c4)nc(-c4ccc5sc6ccc7nc(-c8ccccc8)oc7c6c5c4)n3)c2)cc1 ; c1ccc(-c2nc3ccc4sc5ccc(-c6nc(-c7ccc8c(c7)oc7ccccc78)nc(-c7ccc8c9ccccc9c9ccccc9c8c7)n6)cc5c4c3o2)cc1]

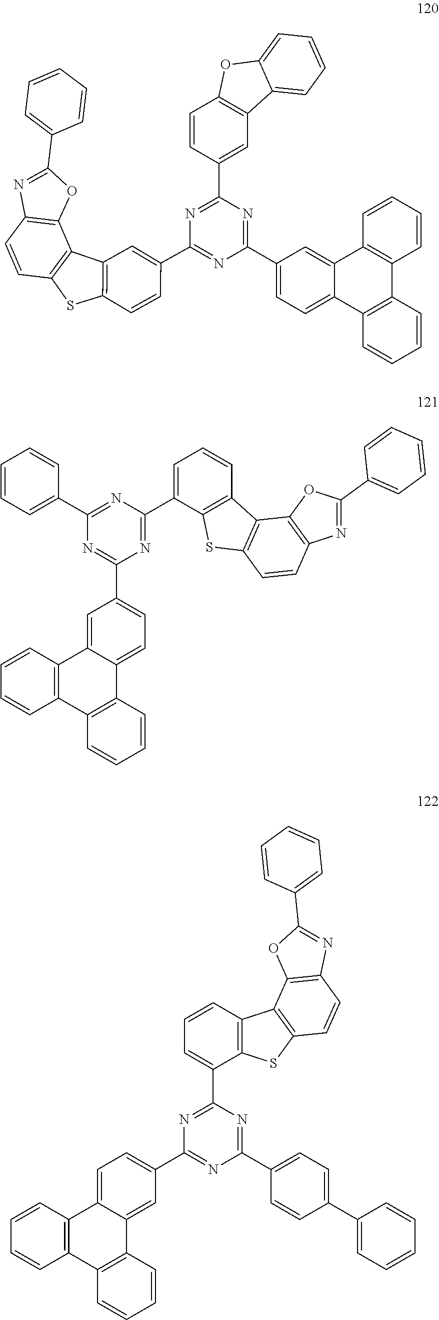 [SMILES: c1ccc(-c2nc3ccc4sc5ccc(-c6nc(-c7ccc8oc9ccccc9c8c7)nc(-c7ccc8c9ccccc9c9ccccc9c8c7)n6)cc5c4c3o2)cc1 ; c1ccc(-c2nc(-c3ccc4c5ccccc5c5ccccc5c4c3)nc(-c3cccc4c3sc3ccc5nc(-c6ccccc6)oc5c34)n2)cc1 ; c1ccc(-c2ccc(-c3nc(-c4ccc5c6ccccc6c6ccccc6c5c4)nc(-c4cccc5c4sc4ccc6nc(-c7ccccc7)oc6c45)n3)cc2)cc1]

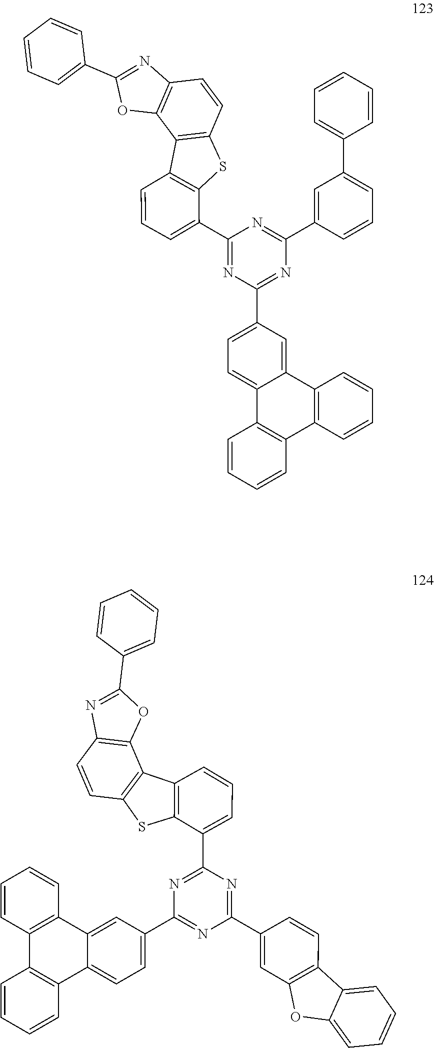 [SMILES: c1ccc(-c2cccc(-c3nc(-c4ccc5c6ccccc6c6ccccc6c5c4)nc(-c4cccc5c4sc4ccc6nc(-c7ccccc7)oc6c45)n3)c2)cc1 ; c1ccc(-c2nc3ccc4sc5c(-c6nc(-c7ccc8c(c7)oc7ccccc78)nc(-c7ccc8c9ccccc9c9ccccc9c8c7)n6)cccc5c4c3o2)cc1]

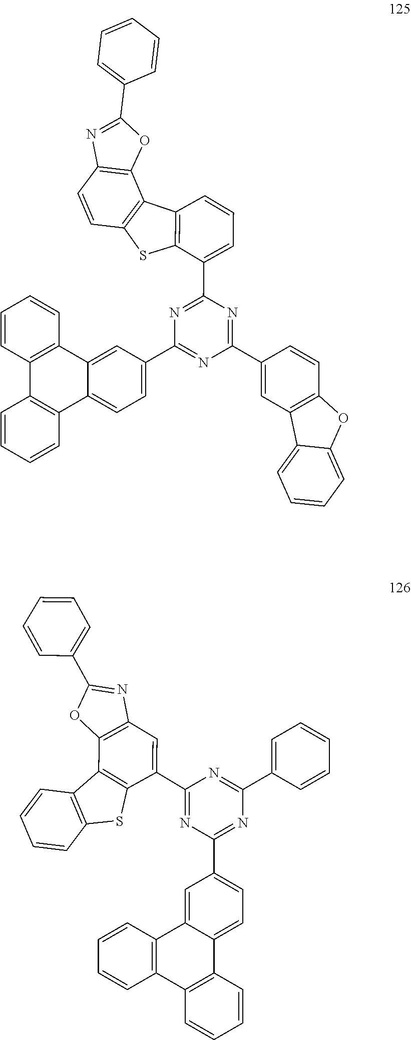 [SMILES: c1ccc(-c2nc3ccc4sc5c(-c6nc(-c7ccc8oc9ccccc9c8c7)nc(-c7ccc8c9ccccc9c9ccccc9c8c7)n6)cccc5c4c3o2)cc1 ; c1ccc(-c2nc(-c3ccc4c5ccccc5c5ccccc5c4c3)nc(-c3cc4nc(-c5ccccc5)oc4c4c3sc3ccccc34)n2)cc1]

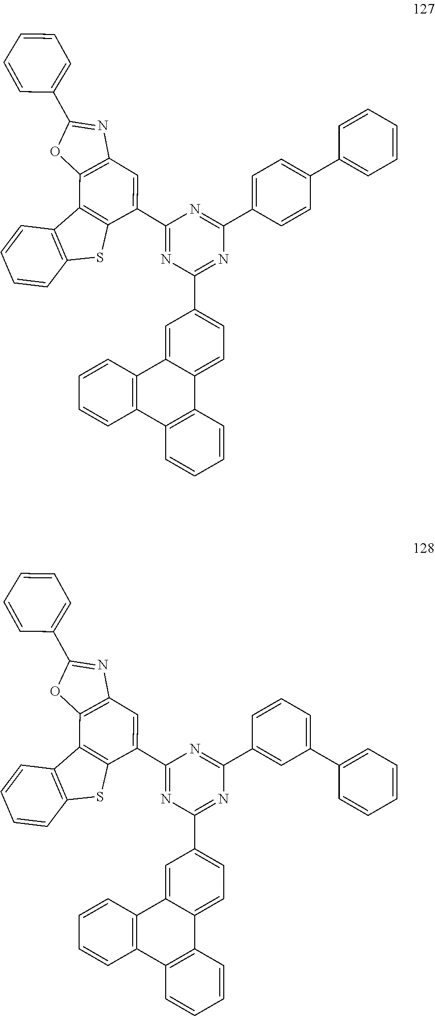 [SMILES: c1ccc(-c2ccc(-c3nc(-c4ccc5c6ccccc6c6ccccc6c5c4)nc(-c4cc5nc(-c6ccccc6)oc5c5c4sc4ccccc45)n3)cc2)cc1 ; c1ccc(-c2cccc(-c3nc(-c4ccc5c6ccccc6c6ccccc6c5c4)nc(-c4cc5nc(-c6ccccc6)oc5c5c4sc4ccccc45)n3)c2)cc1]

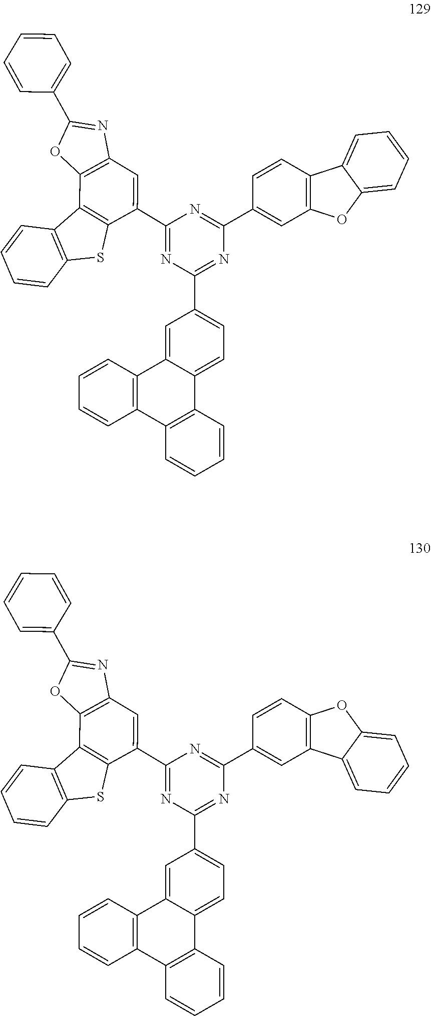 [SMILES: c1ccc(-c2nc3cc(-c4nc(-c5ccc6c(c5)oc5ccccc56)nc(-c5ccc6c7ccccc7c7ccccc7c6c5)n4)c4sc5ccccc5c4c3o2)cc1 ; c1ccc(-c2nc3cc(-c4nc(-c5ccc6oc7ccccc7c6c5)nc(-c5ccc6c7ccccc7c7ccccc7c6c5)n4)c4sc5ccccc5c4c3o2)cc1]

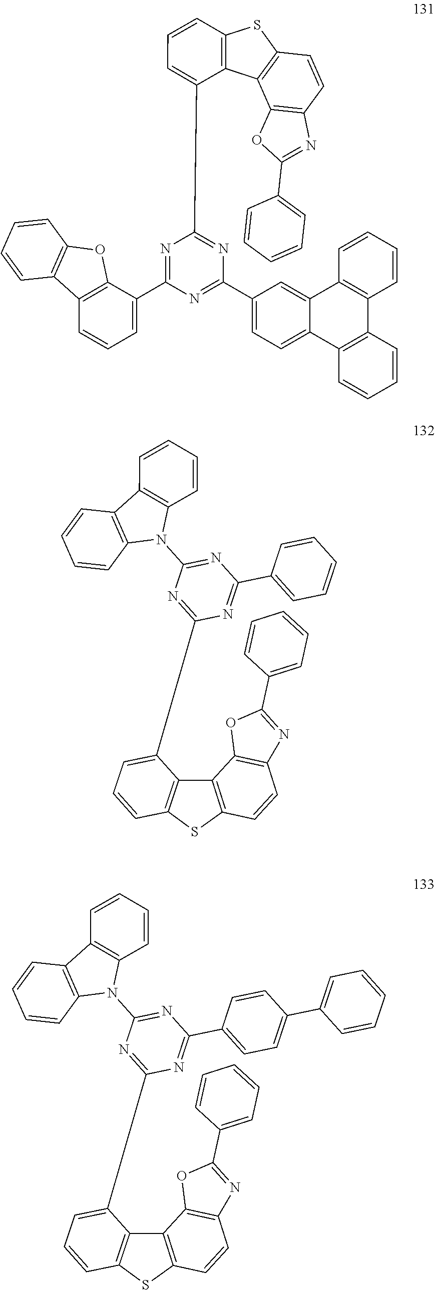 [SMILES: c1ccc(-c2nc3ccc4sc5cccc(-c6nc(-c7ccc8c9ccccc9c9ccccc9c8c7)nc(-c7cccc8c7oc7ccccc78)n6)c5c4c3o2)cc1 ; c1ccc(-c2nc(-c3cccc4sc5ccc6nc(-c7ccccc7)oc6c5c34)nc(-n3c4ccccc4c4ccccc43)n2)cc1 ; c1ccc(-c2ccc(-c3nc(-c4cccc5sc6ccc7nc(-c8ccccc8)oc7c6c45)nc(-n4c5ccccc5c5ccccc54)n3)cc2)cc1]

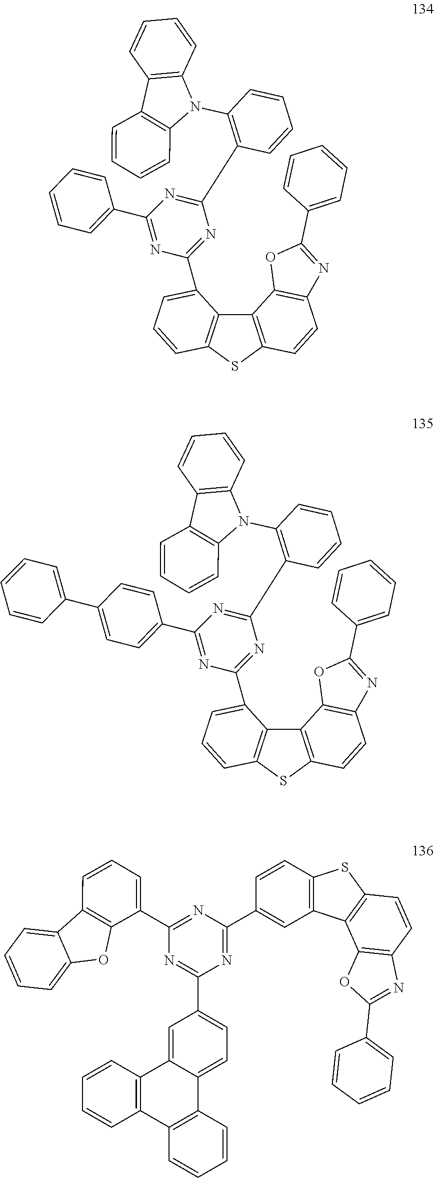 [SMILES: c1ccc(-c2nc(-c3ccccc3-n3c4ccccc4c4ccccc43)nc(-c3cccc4sc5ccc6nc(-c7ccccc7)oc6c5c34)n2)cc1 ; c1ccc(-c2ccc(-c3nc(-c4ccccc4-n4c5ccccc5c5ccccc54)nc(-c4cccc5sc6ccc7nc(-c8ccccc8)oc7c6c45)n3)cc2)cc1 ; c1ccc(-c2nc3ccc4sc5ccc(-c6nc(-c7ccc8c9ccccc9c9ccccc9c8c7)nc(-c7cccc8c7oc7ccccc78)n6)cc5c4c3o2)cc1]

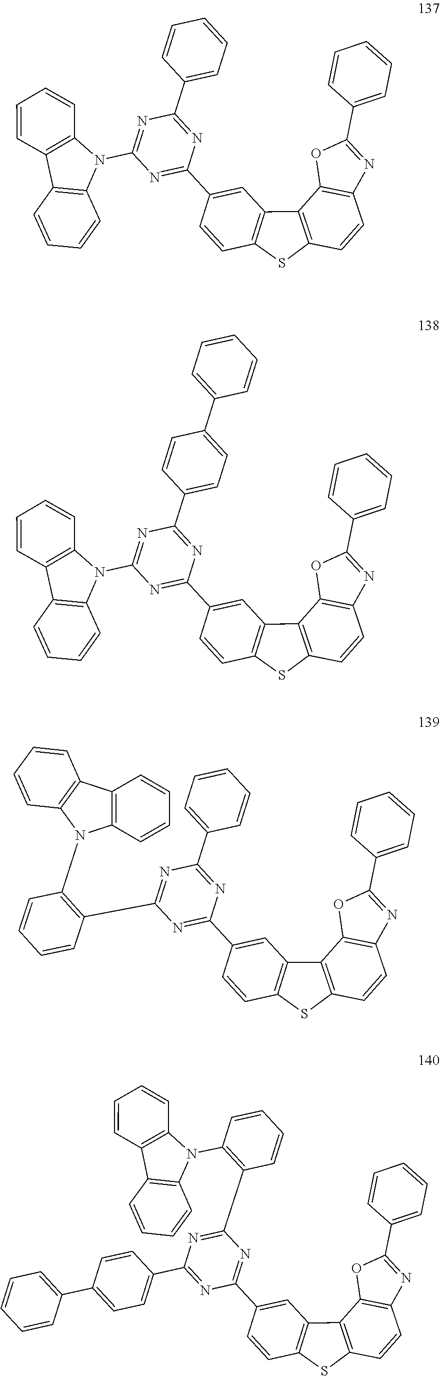 [SMILES: c1ccc(-c2nc(-c3ccc4sc5ccc6nc(-c7ccccc7)oc6c5c4c3)nc(-n3c4ccccc4c4ccccc43)n2)cc1 ; c1ccc(-c2ccc(-c3nc(-c4ccc5sc6ccc7nc(-c8ccccc8)oc7c6c5c4)nc(-n4c5ccccc5c5ccccc54)n3)cc2)cc1 ; c1ccc(-c2nc(-c3ccc4sc5ccc6nc(-c7ccccc7)oc6c5c4c3)nc(-c3ccccc3-n3c4ccccc4c4ccccc43)n2)cc1 ; c1ccc(-c2ccc(-c3nc(-c4ccc5sc6ccc7nc(-c8ccccc8)oc7c6c5c4)nc(-c4ccccc4-n4c5ccccc5c5ccccc54)n3)cc2)cc1]

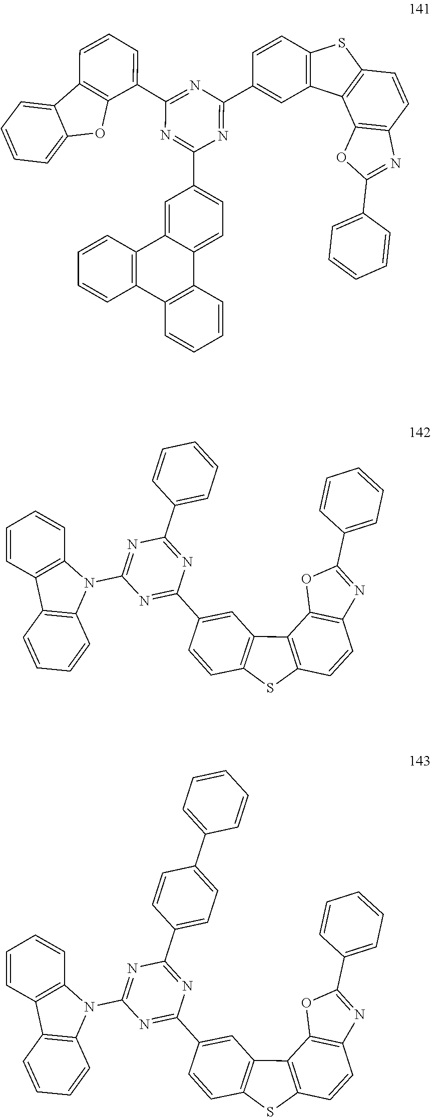 [SMILES: c1ccc(-c2nc3ccc4sc5ccc(-c6nc(-c7ccc8c9ccccc9c9ccccc9c8c7)nc(-c7cccc8c7oc7ccccc78)n6)cc5c4c3o2)cc1 ; c1ccc(-c2nc(-c3ccc4sc5ccc6nc(-c7ccccc7)oc6c5c4c3)nc(-n3c4ccccc4c4ccccc43)n2)cc1 ; c1ccc(-c2ccc(-c3nc(-c4ccc5sc6ccc7nc(-c8ccccc8)oc7c6c5c4)nc(-n4c5ccccc5c5ccccc54)n3)cc2)cc1]

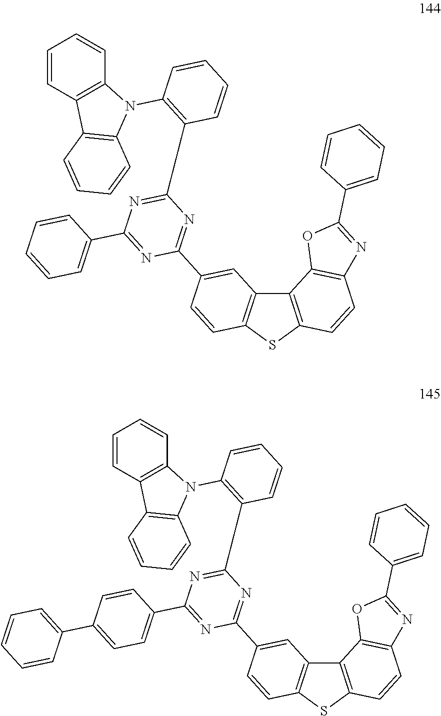 [SMILES: c1ccc(-c2nc(-c3ccc4sc5ccc6nc(-c7ccccc7)oc6c5c4c3)nc(-c3ccccc3-n3c4ccccc4c4ccccc43)n2)cc1 ; c1ccc(-c2ccc(-c3nc(-c4ccc5sc6ccc7nc(-c8ccccc8)oc7c6c5c4)nc(-c4ccccc4-n4c5ccccc5c5ccccc54)n3)cc2)cc1]

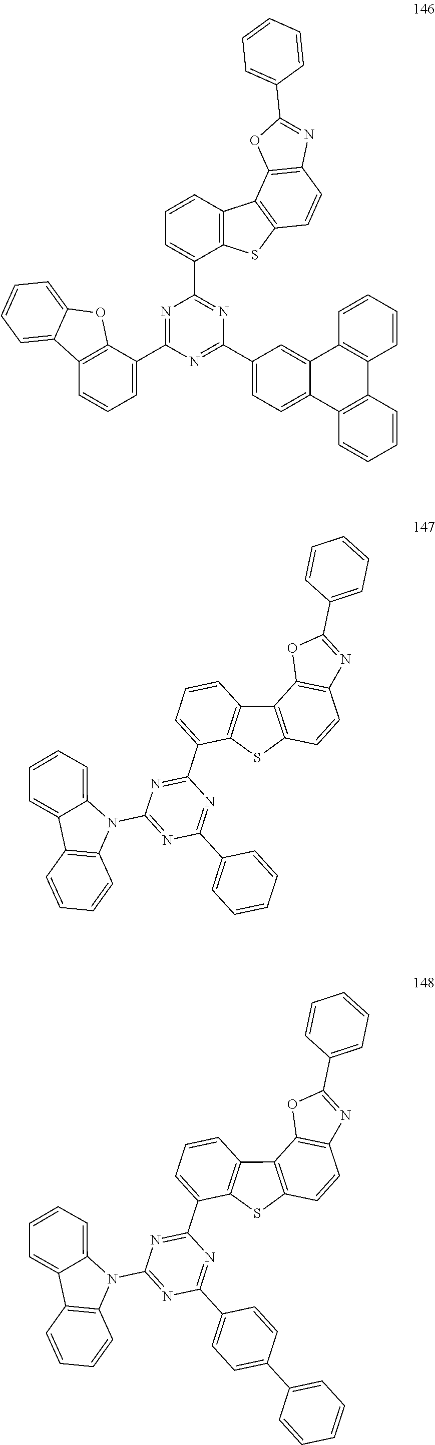 [SMILES: c1ccc(-c2nc3ccc4sc5c(-c6nc(-c7ccc8c9ccccc9c9ccccc9c8c7)nc(-c7cccc8c7oc7ccccc78)n6)cccc5c4c3o2)cc1 ; c1ccc(-c2nc(-c3cccc4c3sc3ccc5nc(-c6ccccc6)oc5c34)nc(-n3c4ccccc4c4ccccc43)n2)cc1 ; c1ccc(-c2ccc(-c3nc(-c4cccc5c4sc4ccc6nc(-c7ccccc7)oc6c45)nc(-n4c5ccccc5c5ccccc54)n3)cc2)cc1]

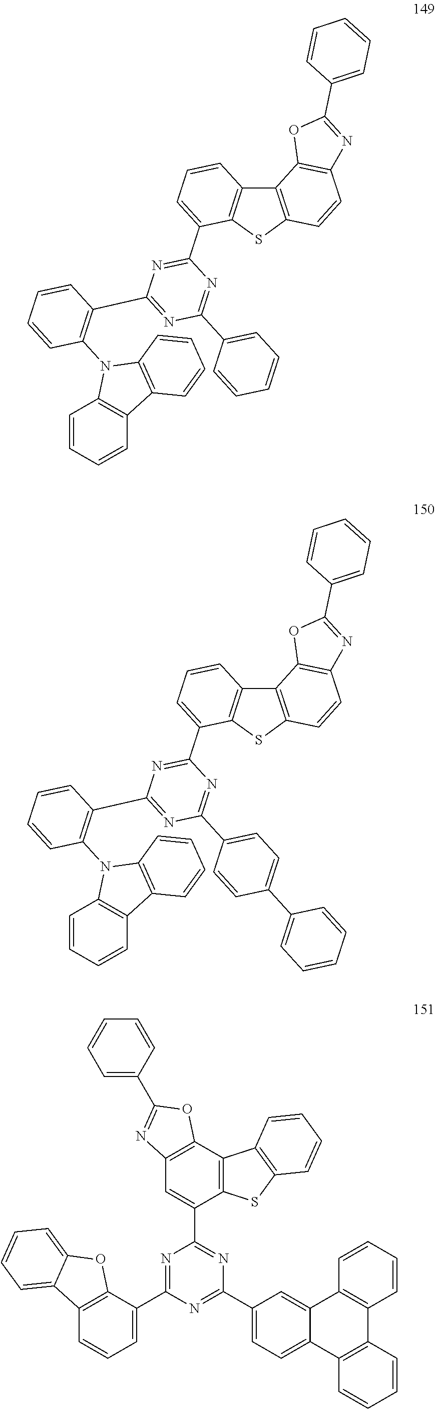 [SMILES: c1ccc(-c2nc(-c3ccccc3-n3c4ccccc4c4ccccc43)nc(-c3cccc4c3sc3ccc5nc(-c6ccccc6)oc5c34)n2)cc1 ; c1ccc(-c2ccc(-c3nc(-c4ccccc4-n4c5ccccc5c5ccccc54)nc(-c4cccc5c4sc4ccc6nc(-c7ccccc7)oc6c45)n3)cc2)cc1 ; c1ccc(-c2nc3cc(-c4nc(-c5ccc6c7ccccc7c7ccccc7c6c5)nc(-c5cccc6c5oc5ccccc56)n4)c4sc5ccccc5c4c3o2)cc1]

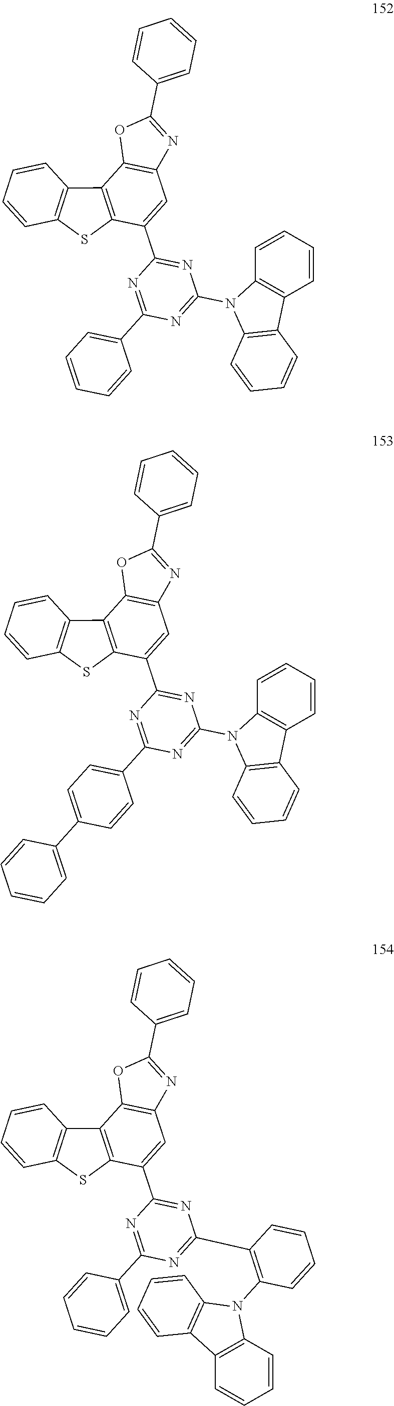 [SMILES: c1ccc(-c2nc(-c3cc4nc(-c5ccccc5)oc4c4c3sc3ccccc34)nc(-n3c4ccccc4c4ccccc43)n2)cc1 ; c1ccc(-c2ccc(-c3nc(-c4cc5nc(-c6ccccc6)oc5c5c4sc4ccccc45)nc(-n4c5ccccc5c5ccccc54)n3)cc2)cc1 ; c1ccc(-c2nc(-c3ccccc3-n3c4ccccc4c4ccccc43)nc(-c3cc4nc(-c5ccccc5)oc4c4c3sc3ccccc34)n2)cc1]

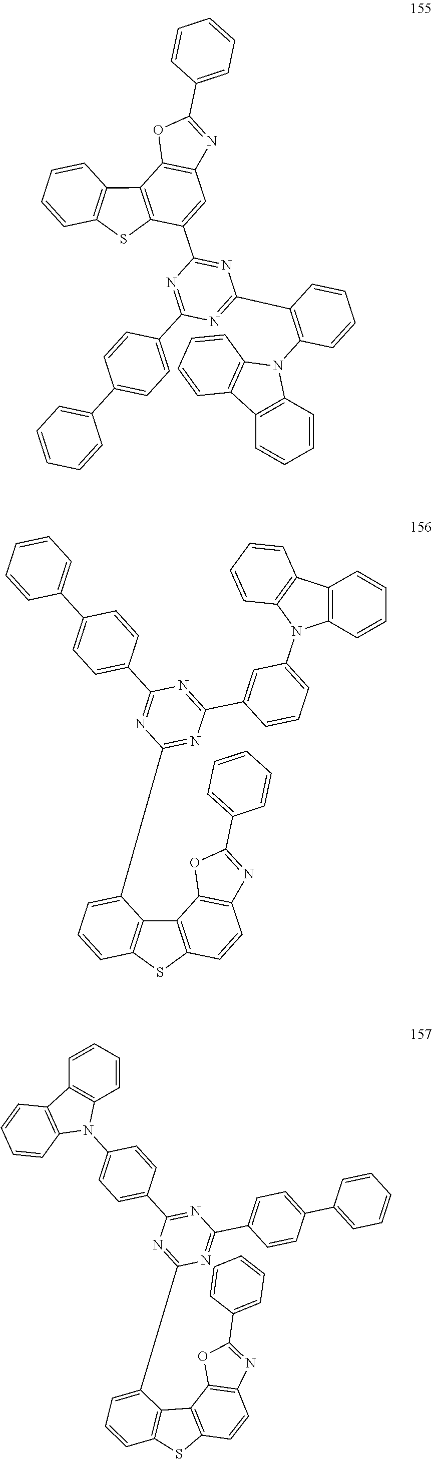 [SMILES: c1ccc(-c2ccc(-c3nc(-c4ccccc4-n4c5ccccc5c5ccccc54)nc(-c4cc5nc(-c6ccccc6)oc5c5c4sc4ccccc45)n3)cc2)cc1 ; c1ccc(-c2ccc(-c3nc(-c4cccc(-n5c6ccccc6c6ccccc65)c4)nc(-c4cccc5sc6ccc7nc(-c8ccccc8)oc7c6c45)n3)cc2)cc1 ; c1ccc(-c2ccc(-c3nc(-c4ccc(-n5c6ccccc6c6ccccc65)cc4)nc(-c4cccc5sc6ccc7nc(-c8ccccc8)oc7c6c45)n3)cc2)cc1]

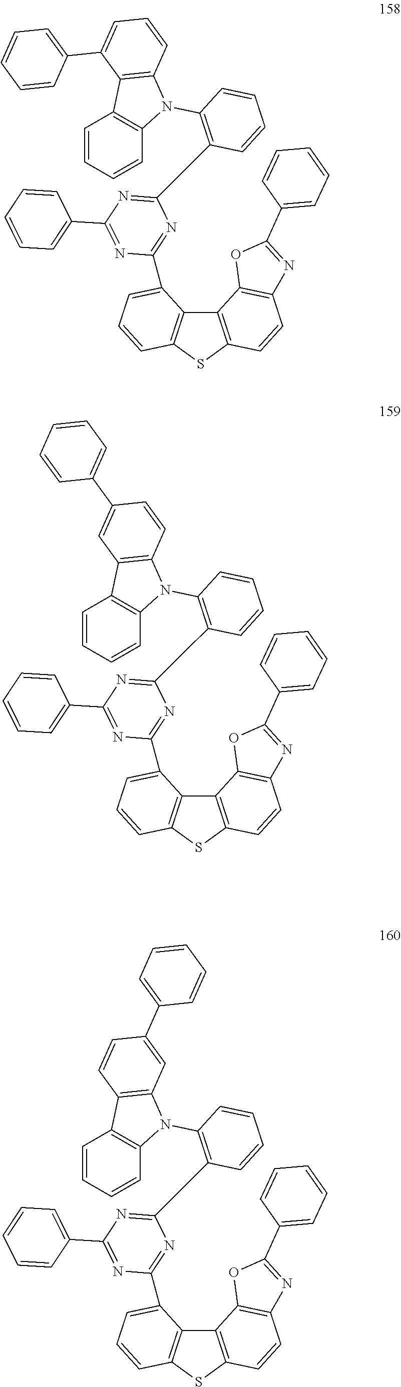 [SMILES: c1ccc(-c2nc(-c3ccccc3-n3c4ccccc4c4c(-c5ccccc5)cccc43)nc(-c3cccc4sc5ccc6nc(-c7ccccc7)oc6c5c34)n2)cc1 ; c1ccc(-c2ccc3c(c2)c2ccccc2n3-c2ccccc2-c2nc(-c3ccccc3)nc(-c3cccc4sc5ccc6nc(-c7ccccc7)oc6c5c34)n2)cc1 ; c1ccc(-c2ccc3c4ccccc4n(-c4ccccc4-c4nc(-c5ccccc5)nc(-c5cccc6sc7ccc8nc(-c9ccccc9)oc8c7c56)n4)c3c2)cc1]

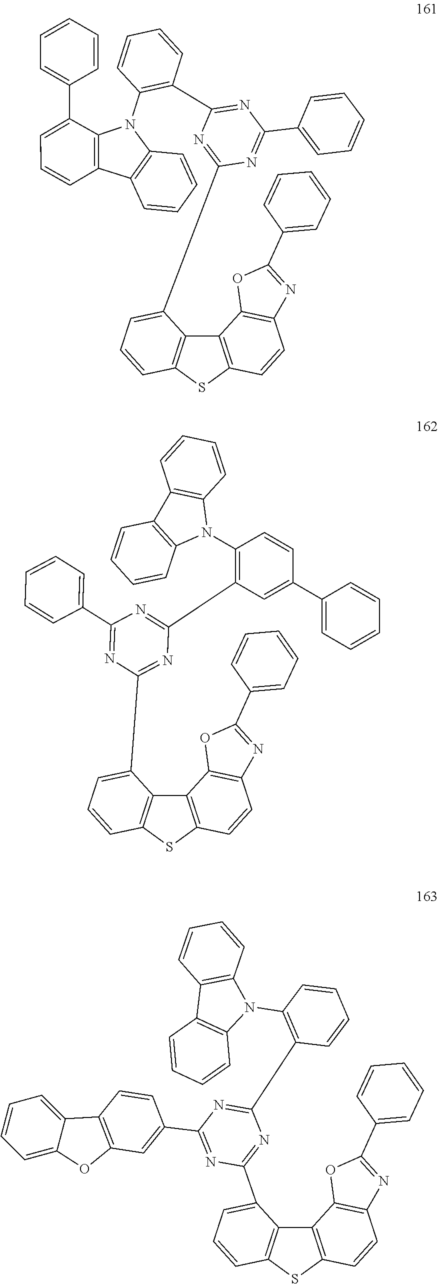 [SMILES: c1ccc(-c2nc(-c3ccccc3-n3c4ccccc4c4cccc(-c5ccccc5)c43)nc(-c3cccc4sc5ccc6nc(-c7ccccc7)oc6c5c34)n2)cc1 ; c1ccc(-c2ccc(-n3c4ccccc4c4ccccc43)c(-c3nc(-c4ccccc4)nc(-c4cccc5sc6ccc7nc(-c8ccccc8)oc7c6c45)n3)c2)cc1 ; c1ccc(-c2nc3ccc4sc5cccc(-c6nc(-c7ccc8c(c7)oc7ccccc78)nc(-c7ccccc7-n7c8ccccc8c8ccccc87)n6)c5c4c3o2)cc1]

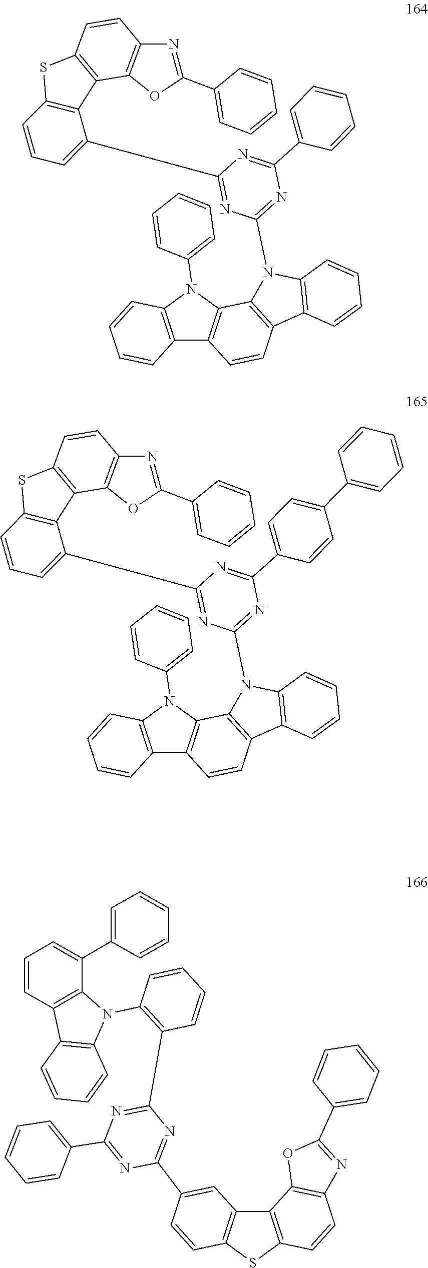 [SMILES: c1ccc(-c2nc(-c3cccc4sc5ccc6nc(-c7ccccc7)oc6c5c34)nc(-n3c4ccccc4c4ccc5c6ccccc6n(-c6ccccc6)c5c43)n2)cc1 ; c1ccc(-c2ccc(-c3nc(-c4cccc5sc6ccc7nc(-c8ccccc8)oc7c6c45)nc(-n4c5ccccc5c5ccc6c7ccccc7n(-c7ccccc7)c6c54)n3)cc2)cc1 ; c1ccc(-c2nc(-c3ccc4sc5ccc6nc(-c7ccccc7)oc6c5c4c3)nc(-c3ccccc3-n3c4ccccc4c4cccc(-c5ccccc5)c43)n2)cc1]

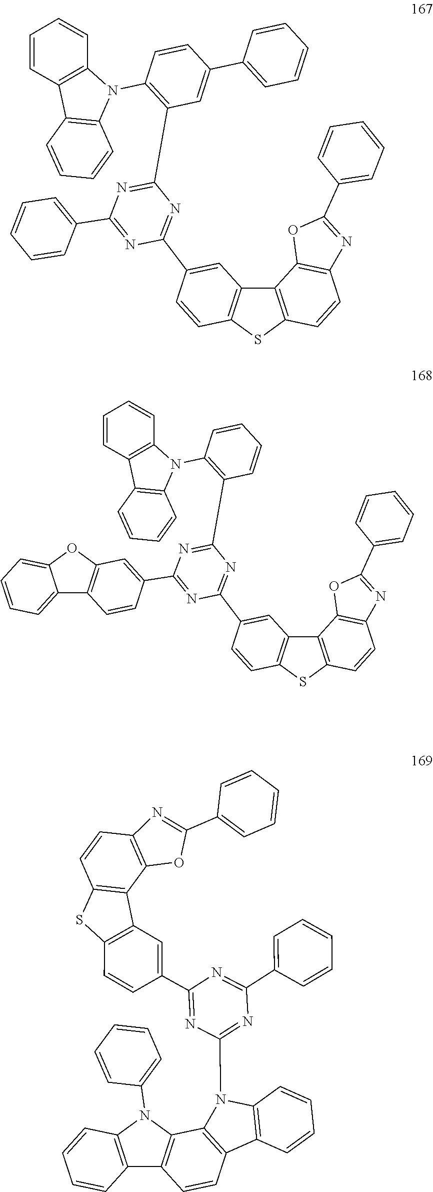 [SMILES: c1ccc(-c2ccc(-n3c4ccccc4c4ccccc43)c(-c3nc(-c4ccccc4)nc(-c4ccc5sc6ccc7nc(-c8ccccc8)oc7c6c5c4)n3)c2)cc1 ; c1ccc(-c2nc3ccc4sc5ccc(-c6nc(-c7ccc8c(c7)oc7ccccc78)nc(-c7ccccc7-n7c8ccccc8c8ccccc87)n6)cc5c4c3o2)cc1 ; c1ccc(-c2nc(-c3ccc4sc5ccc6nc(-c7ccccc7)oc6c5c4c3)nc(-n3c4ccccc4c4ccc5c6ccccc6n(-c6ccccc6)c5c43)n2)cc1]

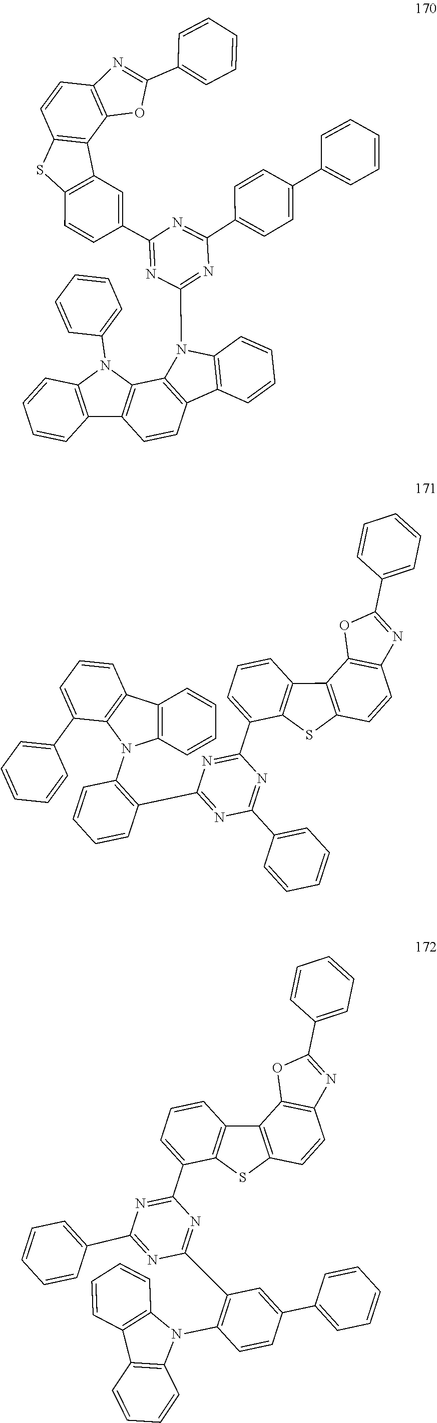 [SMILES: c1ccc(-c2ccc(-c3nc(-c4ccc5sc6ccc7nc(-c8ccccc8)oc7c6c5c4)nc(-n4c5ccccc5c5ccc6c7ccccc7n(-c7ccccc7)c6c54)n3)cc2)cc1 ; c1ccc(-c2nc(-c3ccccc3-n3c4ccccc4c4cccc(-c5ccccc5)c43)nc(-c3cccc4c3sc3ccc5nc(-c6ccccc6)oc5c34)n2)cc1 ; c1ccc(-c2ccc(-n3c4ccccc4c4ccccc43)c(-c3nc(-c4ccccc4)nc(-c4cccc5c4sc4ccc6nc(-c7ccccc7)oc6c45)n3)c2)cc1]

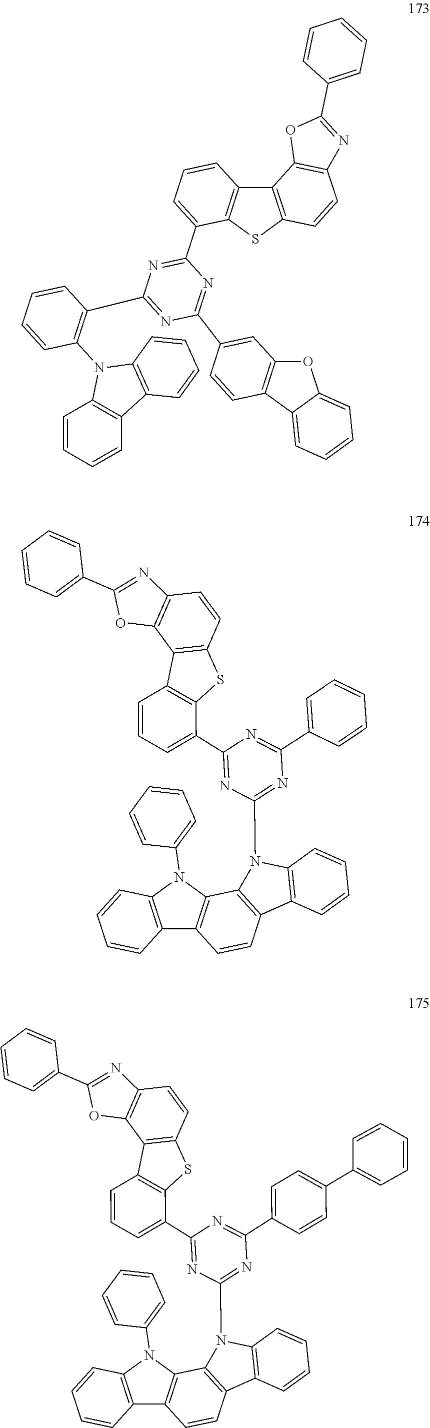 [SMILES: c1ccc(-c2nc3ccc4sc5c(-c6nc(-c7ccc8c(c7)oc7ccccc78)nc(-c7ccccc7-n7c8ccccc8c8ccccc87)n6)cccc5c4c3o2)cc1 ; c1ccc(-c2nc(-c3cccc4c3sc3ccc5nc(-c6ccccc6)oc5c34)nc(-n3c4ccccc4c4ccc5c6ccccc6n(-c6ccccc6)c5c43)n2)cc1 ; c1ccc(-c2ccc(-c3nc(-c4cccc5c4sc4ccc6nc(-c7ccccc7)oc6c45)nc(-n4c5ccccc5c5ccc6c7ccccc7n(-c7ccccc7)c6c54)n3)cc2)cc1]

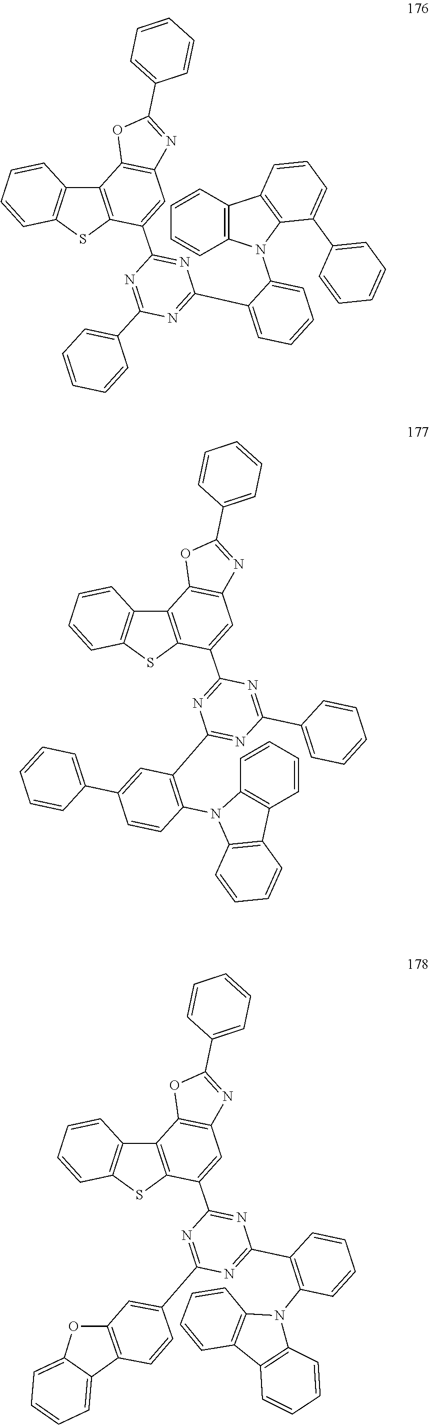 [SMILES: c1ccc(-c2nc(-c3ccccc3-n3c4ccccc4c4cccc(-c5ccccc5)c43)nc(-c3cc4nc(-c5ccccc5)oc4c4c3sc3ccccc34)n2)cc1 ; c1ccc(-c2ccc(-n3c4ccccc4c4ccccc43)c(-c3nc(-c4ccccc4)nc(-c4cc5nc(-c6ccccc6)oc5c5c4sc4ccccc45)n3)c2)cc1 ; c1ccc(-c2nc3cc(-c4nc(-c5ccc6c(c5)oc5ccccc56)nc(-c5ccccc5-n5c6ccccc6c6ccccc65)n4)c4sc5ccccc5c4c3o2)cc1]

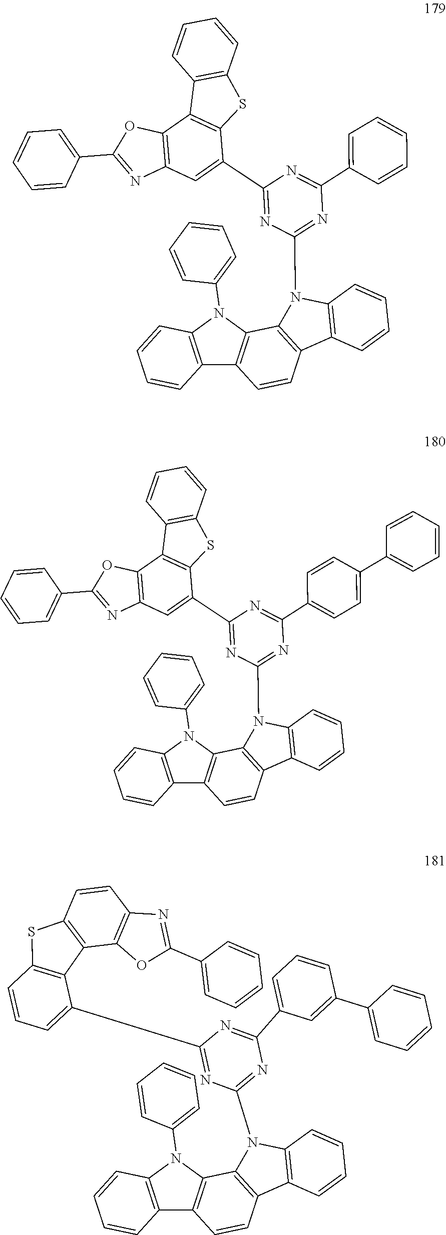 [SMILES: c1ccc(-c2nc(-c3cc4nc(-c5ccccc5)oc4c4c3sc3ccccc34)nc(-n3c4ccccc4c4ccc5c6ccccc6n(-c6ccccc6)c5c43)n2)cc1 ; c1ccc(-c2ccc(-c3nc(-c4cc5nc(-c6ccccc6)oc5c5c4sc4ccccc45)nc(-n4c5ccccc5c5ccc6c7ccccc7n(-c7ccccc7)c6c54)n3)cc2)cc1 ; c1ccc(-c2cccc(-c3nc(-c4cccc5sc6ccc7nc(-c8ccccc8)oc7c6c45)nc(-n4c5ccccc5c5ccc6c7ccccc7n(-c7ccccc7)c6c54)n3)c2)cc1]

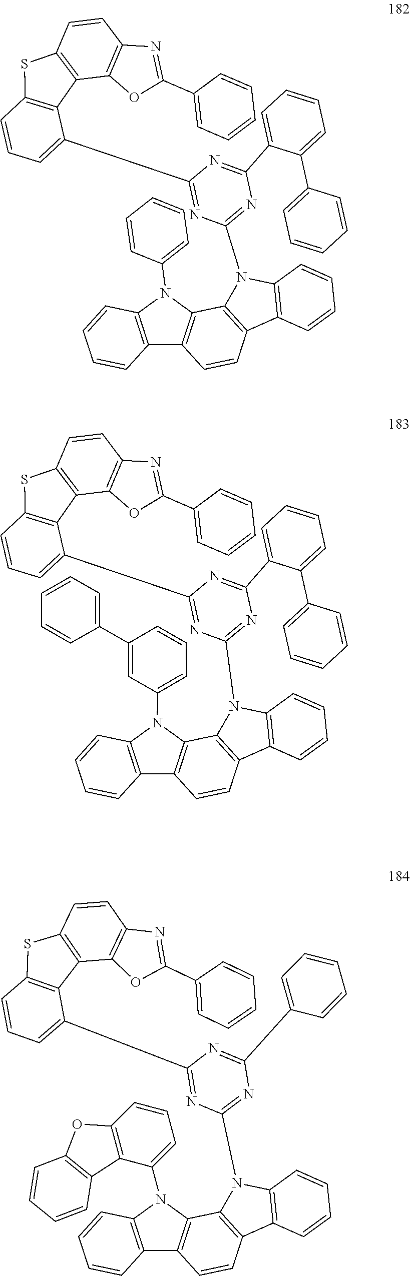 [SMILES: c1ccc(-c2nc3ccc4sc5cccc(-c6nc(-c7ccccc7-c7ccccc7)nc(-n7c8ccccc8c8ccc9c%10ccccc%10n(-c%10ccccc%10)c9c87)n6)c5c4c3o2)cc1 ; c1ccc(-c2cccc(-n3c4ccccc4c4ccc5c6ccccc6n(-c6nc(-c7ccccc7-c7ccccc7)nc(-c7cccc8sc9ccc%10nc(-c%11ccccc%11)oc%10c9c78)n6)c5c43)c2)cc1 ; c1ccc(-c2nc(-c3cccc4sc5ccc6nc(-c7ccccc7)oc6c5c34)nc(-n3c4ccccc4c4ccc5c6ccccc6n(-c6cccc7oc8ccccc8c67)c5c43)n2)cc1]

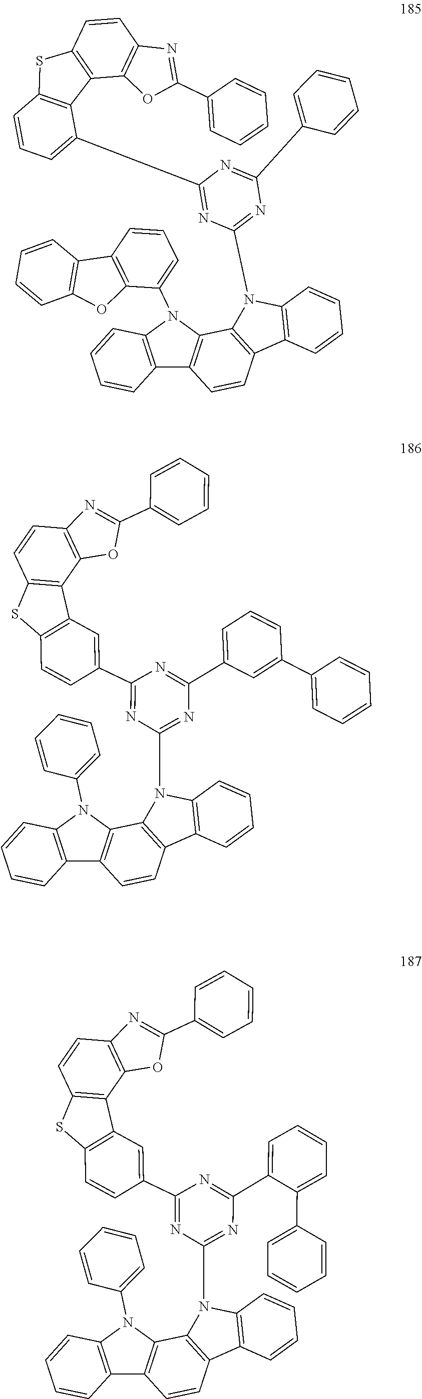 [SMILES: c1ccc(-c2nc(-c3cccc4sc5ccc6nc(-c7ccccc7)oc6c5c34)nc(-n3c4ccccc4c4ccc5c6ccccc6n(-c6cccc7c6oc6ccccc67)c5c43)n2)cc1 ; c1ccc(-c2cccc(-c3nc(-c4ccc5sc6ccc7nc(-c8ccccc8)oc7c6c5c4)nc(-n4c5ccccc5c5ccc6c7ccccc7n(-c7ccccc7)c6c54)n3)c2)cc1 ; c1ccc(-c2nc3ccc4sc5ccc(-c6nc(-c7ccccc7-c7ccccc7)nc(-n7c8ccccc8c8ccc9c%10ccccc%10n(-c%10ccccc%10)c9c87)n6)cc5c4c3o2)cc1]

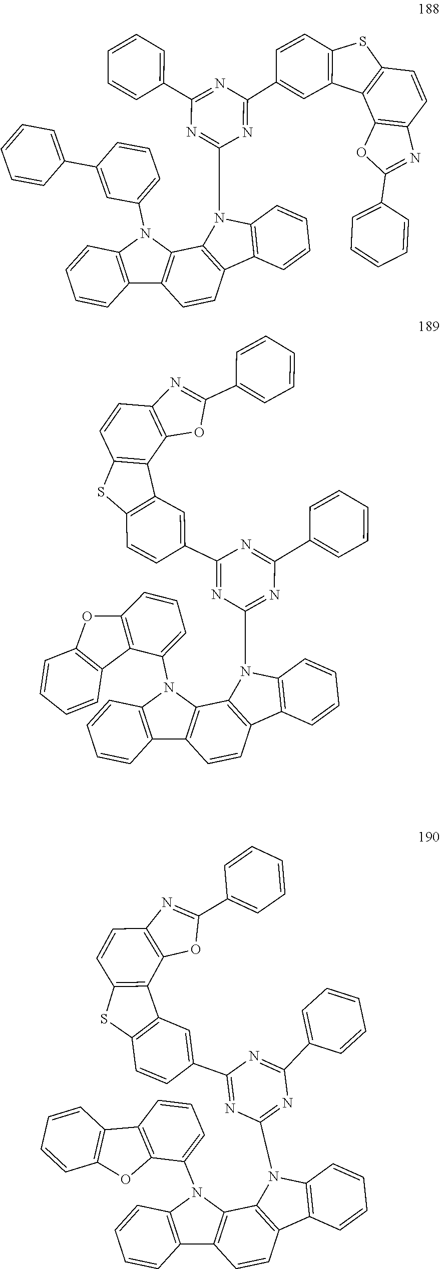 [SMILES: c1ccc(-c2cccc(-n3c4ccccc4c4ccc5c6ccccc6n(-c6nc(-c7ccccc7)nc(-c7ccc8sc9ccc%10nc(-c%11ccccc%11)oc%10c9c8c7)n6)c5c43)c2)cc1 ; c1ccc(-c2nc(-c3ccc4sc5ccc6nc(-c7ccccc7)oc6c5c4c3)nc(-n3c4ccccc4c4ccc5c6ccccc6n(-c6cccc7oc8ccccc8c67)c5c43)n2)cc1 ; c1ccc(-c2nc(-c3ccc4sc5ccc6nc(-c7ccccc7)oc6c5c4c3)nc(-n3c4ccccc4c4ccc5c6ccccc6n(-c6cccc7c6oc6ccccc67)c5c43)n2)cc1]

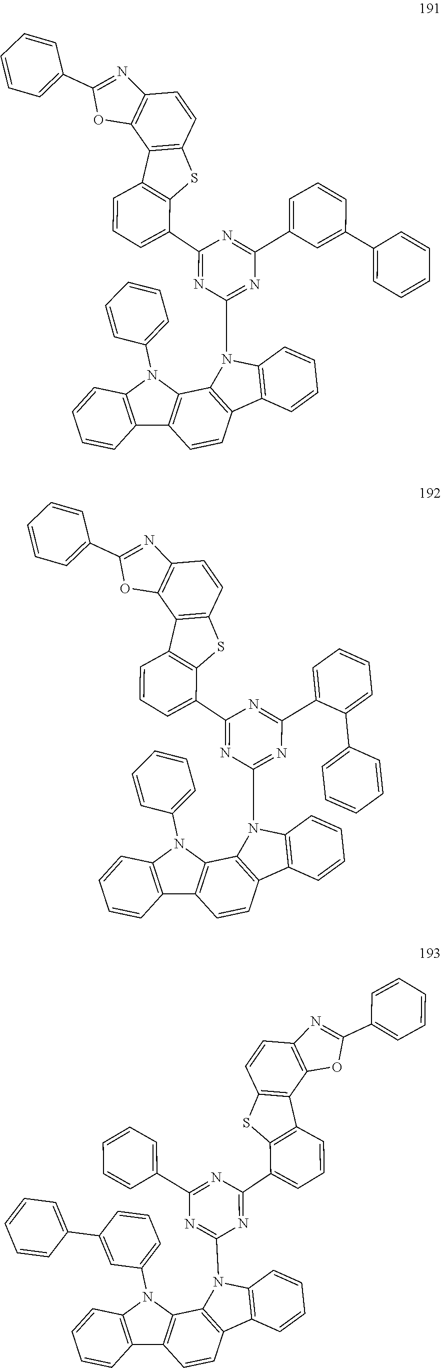 [SMILES: c1ccc(-c2cccc(-c3nc(-c4cccc5c4sc4ccc6nc(-c7ccccc7)oc6c45)nc(-n4c5ccccc5c5ccc6c7ccccc7n(-c7ccccc7)c6c54)n3)c2)cc1 ; c1ccc(-c2nc3ccc4sc5c(-c6nc(-c7ccccc7-c7ccccc7)nc(-n7c8ccccc8c8ccc9c%10ccccc%10n(-c%10ccccc%10)c9c87)n6)cccc5c4c3o2)cc1 ; c1ccc(-c2cccc(-n3c4ccccc4c4ccc5c6ccccc6n(-c6nc(-c7ccccc7)nc(-c7cccc8c7sc7ccc9nc(-c%10ccccc%10)oc9c78)n6)c5c43)c2)cc1]

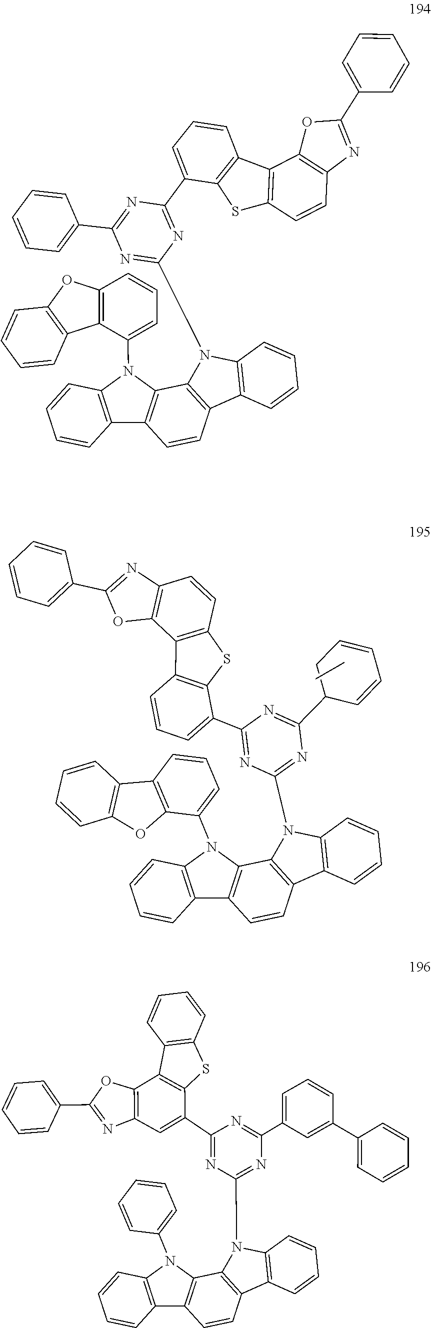

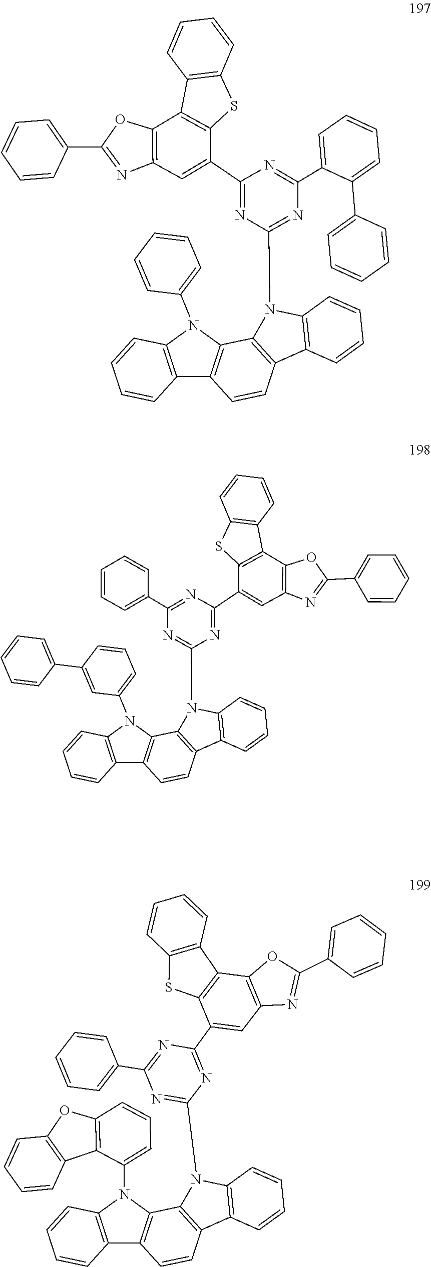 [SMILES: c1ccc(-c2nc3cc(-c4nc(-c5ccccc5-c5ccccc5)nc(-n5c6ccccc6c6ccc7c8ccccc8n(-c8ccccc8)c7c65)n4)c4sc5ccccc5c4c3o2)cc1 ; c1ccc(-c2cccc(-n3c4ccccc4c4ccc5c6ccccc6n(-c6nc(-c7ccccc7)nc(-c7cc8nc(-c9ccccc9)oc8c8c7sc7ccccc78)n6)c5c43)c2)cc1 ; c1ccc(-c2nc(-c3cc4nc(-c5ccccc5)oc4c4c3sc3ccccc34)nc(-n3c4ccccc4c4ccc5c6ccccc6n(-c6cccc7oc8ccccc8c67)c5c43)n2)cc1]

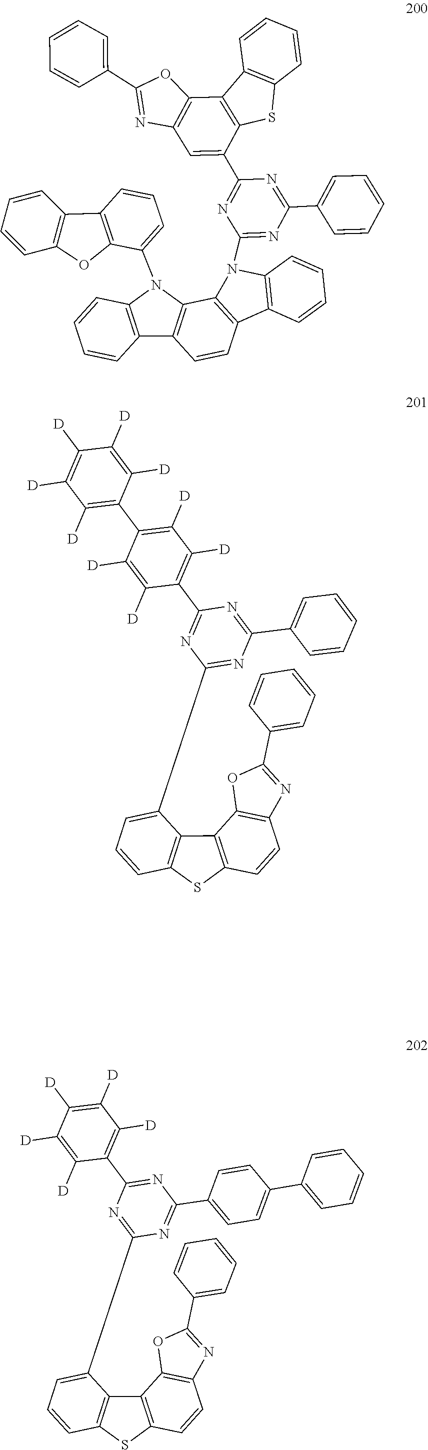 [SMILES: c1ccc(-c2nc(-c3cc4nc(-c5ccccc5)oc4c4c3sc3ccccc34)nc(-n3c4ccccc4c4ccc5c6ccccc6n(-c6cccc7c6oc6ccccc67)c5c43)n2)cc1 ; [2H]c1c([2H])c([2H])c(-c2c([2H])c([2H])c(-c3nc(-c4ccccc4)nc(-c4cccc5sc6ccc7nc(-c8ccccc8)oc7c6c45)n3)c([2H])c2[2H])c([2H])c1[2H] ; [2H]c1c([2H])c([2H])c(-c2nc(-c3ccc(-c4ccccc4)cc3)nc(-c3cccc4sc5ccc6nc(-c7ccccc7)oc6c5c34)n2)c([2H])c1[2H]]

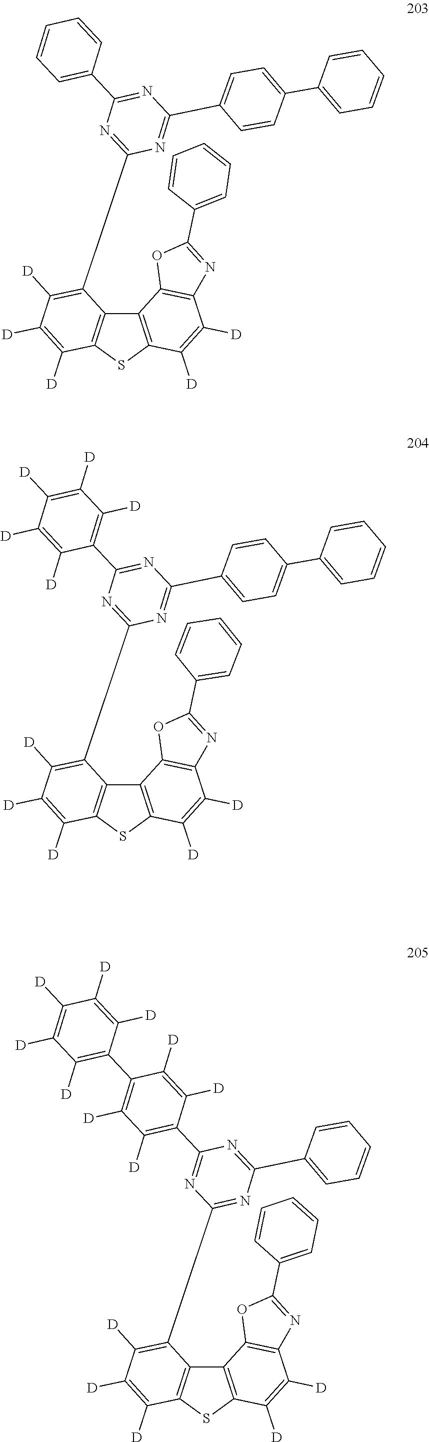 [SMILES: [2H]c1c([2H])c(-c2nc(-c3ccccc3)nc(-c3ccc(-c4ccccc4)cc3)n2)c2c(sc3c([2H])c([2H])c4nc(-c5ccccc5)oc4c32)c1[2H] ; [2H]c1c([2H])c([2H])c(-c2nc(-c3ccc(-c4ccccc4)cc3)nc(-c3c([2H])c([2H])c([2H])c4sc5c([2H])c([2H])c6nc(-c7ccccc7)oc6c5c34)n2)c([2H])c1[2H] ; [2H]c1c([2H])c([2H])c(-c2c([2H])c([2H])c(-c3nc(-c4ccccc4)nc(-c4c([2H])c([2H])c([2H])c5sc6c([2H])c([2H])c7nc(-c8ccccc8)oc7c6c45)n3)c([2H])c2[2H])c([2H])c1[2H]]

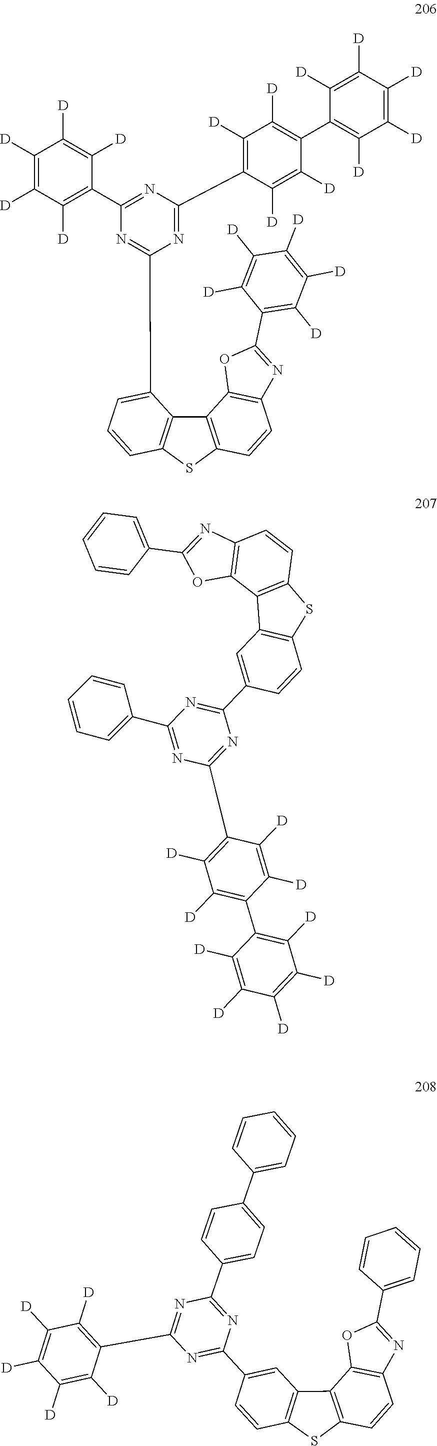 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3cccc4sc5ccc6nc(-c7c([2H])c([2H])c([2H])c([2H])c7[2H])oc6c5c34)n2)c([2H])c1[2H] ; [2H]c1c([2H])c([2H])c(-c2c([2H])c([2H])c(-c3nc(-c4ccccc4)nc(-c4ccc5sc6ccc7nc(-c8ccccc8)oc7c6c5c4)n3)c([2H])c2[2H])c([2H])c1[2H] ; [2H]c1c([2H])c([2H])c(-c2nc(-c3ccc(-c4ccccc4)cc3)nc(-c3ccc4sc5ccc6nc(-c7ccccc7)oc6c5c4c3)n2)c([2H])c1[2H]]

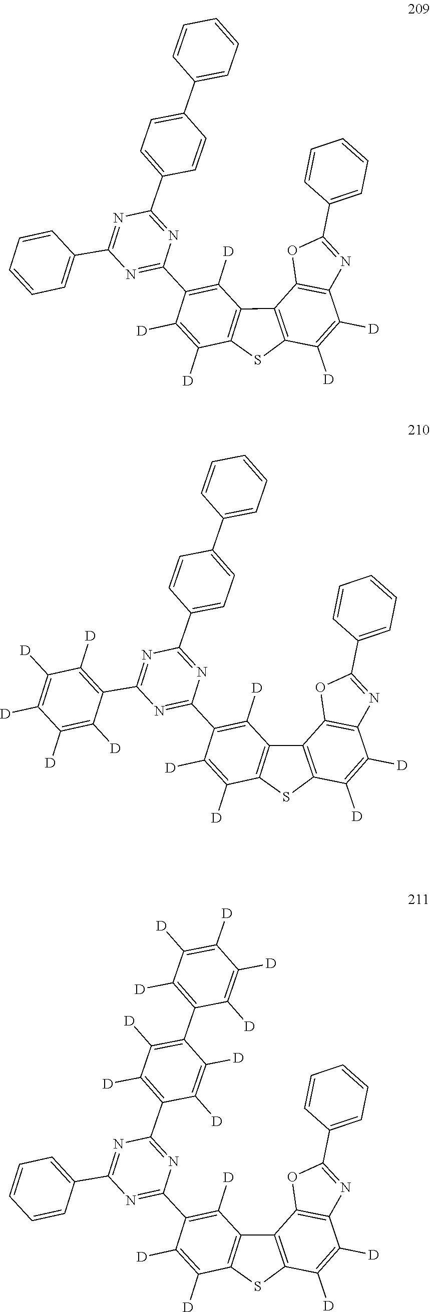 [SMILES: [2H]c1c(-c2nc(-c3ccccc3)nc(-c3ccc(-c4ccccc4)cc3)n2)c([2H])c2c(sc3c([2H])c([2H])c4nc(-c5ccccc5)oc4c32)c1[2H] ; [2H]c1c([2H])c([2H])c(-c2nc(-c3ccc(-c4ccccc4)cc3)nc(-c3c([2H])c([2H])c4sc5c([2H])c([2H])c6nc(-c7ccccc7)oc6c5c4c3[2H])n2)c([2H])c1[2H] ; [2H]c1c([2H])c([2H])c(-c2c([2H])c([2H])c(-c3nc(-c4ccccc4)nc(-c4c([2H])c([2H])c5sc6c([2H])c([2H])c7nc(-c8ccccc8)oc7c6c5c4[2H])n3)c([2H])c2[2H])c([2H])c1[2H]]

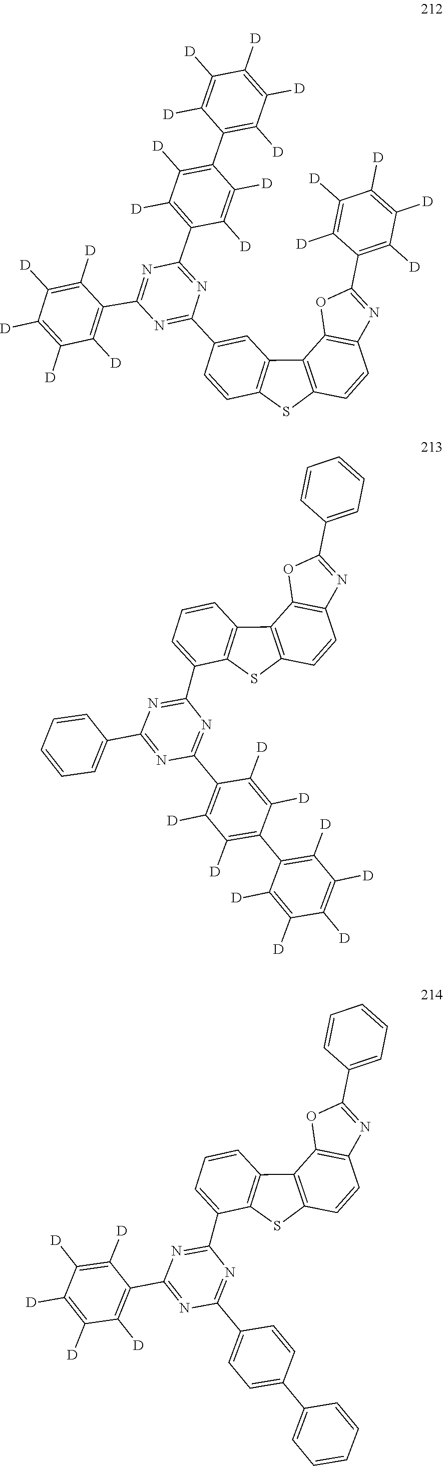 [SMILES: [2H]c1c([2H])c([2H])c(-c2nc(-c3ccc4sc5ccc6nc(-c7c([2H])c([2H])c([2H])c([2H])c7[2H])oc6c5c4c3)nc(-c3c([2H])c([2H])c(-c4c([2H])c([2H])c([2H])c([2H])c4[2H])c([2H])c3[2H])n2)c([2H])c1[2H] ; [2H]c1c([2H])c([2H])c(-c2c([2H])c([2H])c(-c3nc(-c4ccccc4)nc(-c4cccc5c4sc4ccc6nc(-c7ccccc7)oc6c45)n3)c([2H])c2[2H])c([2H])c1[2H] ; [2H]c1c([2H])c([2H])c(-c2nc(-c3ccc(-c4ccccc4)cc3)nc(-c3cccc4c3sc3ccc5nc(-c6ccccc6)oc5c34)n2)c([2H])c1[2H]]

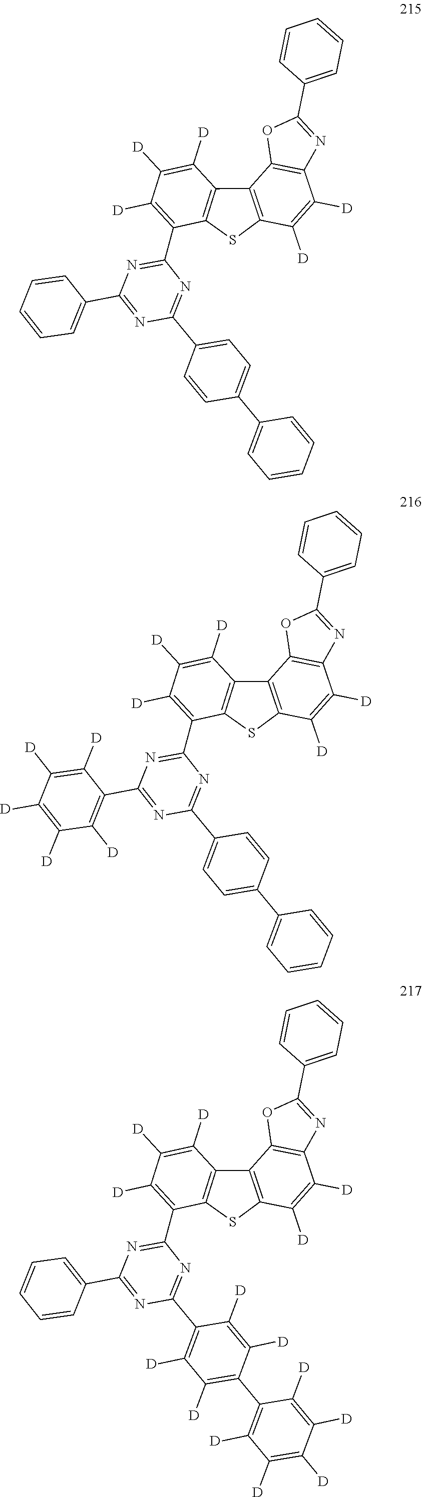

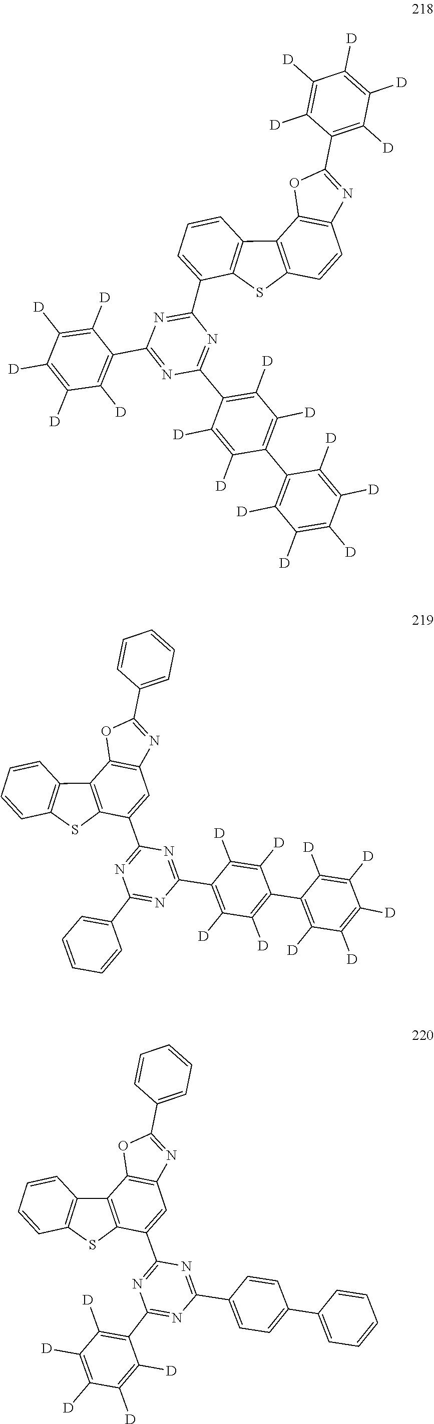 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3cccc4c3sc3ccc5nc(-c6c([2H])c([2H])c([2H])c([2H])c6[2H])oc5c34)n2)c([2H])c1[2H] ; [2H]c1c([2H])c([2H])c(-c2c([2H])c([2H])c(-c3nc(-c4ccccc4)nc(-c4cc5nc(-c6ccccc6)oc5c5c4sc4ccccc45)n3)c([2H])c2[2H])c([2H])c1[2H] ; [2H]c1c([2H])c([2H])c(-c2nc(-c3ccc(-c4ccccc4)cc3)nc(-c3cc4nc(-c5ccccc5)oc4c4c3sc3ccccc34)n2)c([2H])c1[2H]]

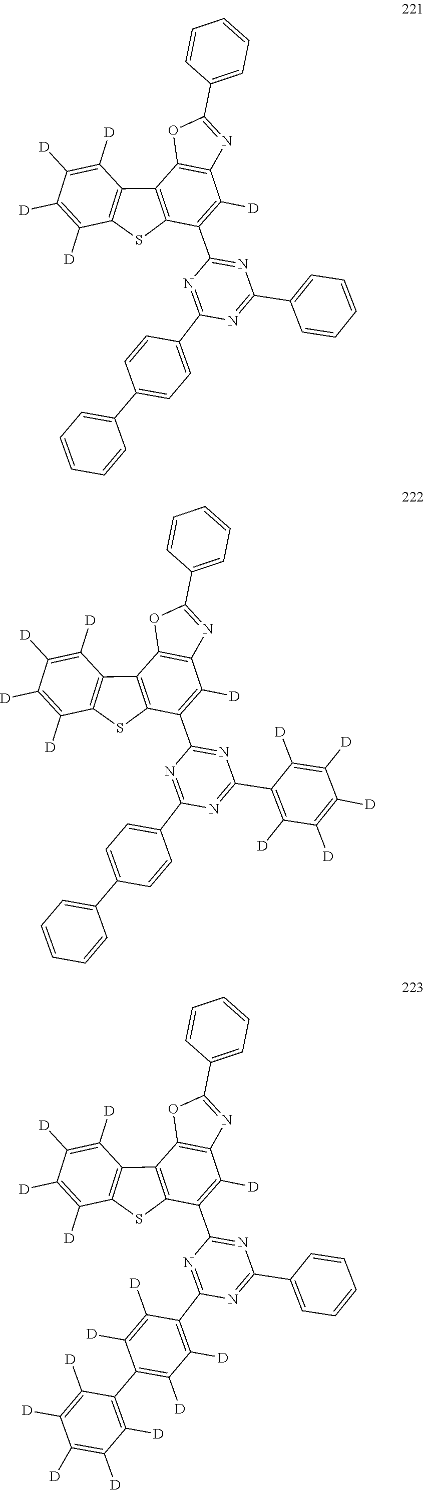 [SMILES: [2H]c1c([2H])c([2H])c2c(sc3c(-c4nc(-c5ccccc5)nc(-c5ccc(-c6ccccc6)cc5)n4)c([2H])c4nc(-c5ccccc5)oc4c32)c1[2H] ; [2H]c1c([2H])c([2H])c(-c2nc(-c3ccc(-c4ccccc4)cc3)nc(-c3c([2H])c4nc(-c5ccccc5)oc4c4c3sc3c([2H])c([2H])c([2H])c([2H])c34)n2)c([2H])c1[2H] ; [2H]c1c([2H])c([2H])c(-c2c([2H])c([2H])c(-c3nc(-c4ccccc4)nc(-c4c([2H])c5nc(-c6ccccc6)oc5c5c4sc4c([2H])c([2H])c([2H])c([2H])c45)n3)c([2H])c2[2H])c([2H])c1[2H]]

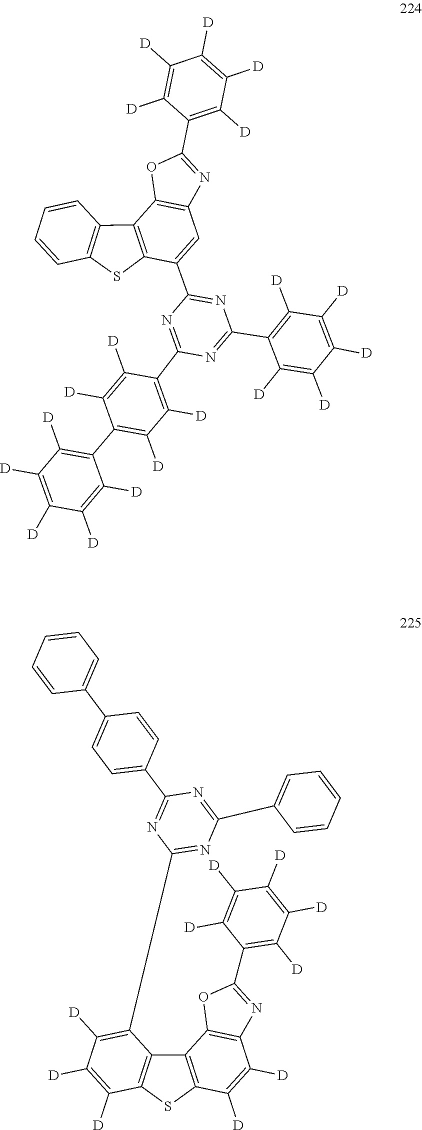 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3cc4nc(-c5c([2H])c([2H])c([2H])c([2H])c5[2H])oc4c4c3sc3ccccc34)n2)c([2H])c1[2H] ; [2H]c1c([2H])c([2H])c(-c2nc3c([2H])c([2H])c4sc5c([2H])c([2H])c([2H])c(-c6nc(-c7ccccc7)nc(-c7ccc(-c8ccccc8)cc7)n6)c5c4c3o2)c([2H])c1[2H]]

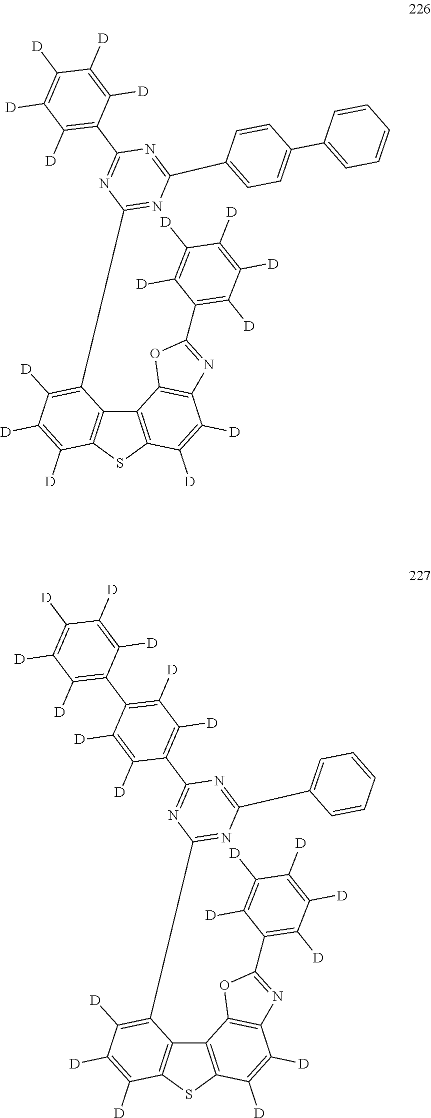 [SMILES: [2H]c1c([2H])c([2H])c(-c2nc(-c3ccc(-c4ccccc4)cc3)nc(-c3c([2H])c([2H])c([2H])c4sc5c([2H])c([2H])c6nc(-c7c([2H])c([2H])c([2H])c([2H])c7[2H])oc6c5c34)n2)c([2H])c1[2H] ; [2H]c1c([2H])c([2H])c(-c2nc3c([2H])c([2H])c4sc5c([2H])c([2H])c([2H])c(-c6nc(-c7ccccc7)nc(-c7c([2H])c([2H])c(-c8c([2H])c([2H])c([2H])c([2H])c8[2H])c([2H])c7[2H])n6)c5c4c3o2)c([2H])c1[2H]]

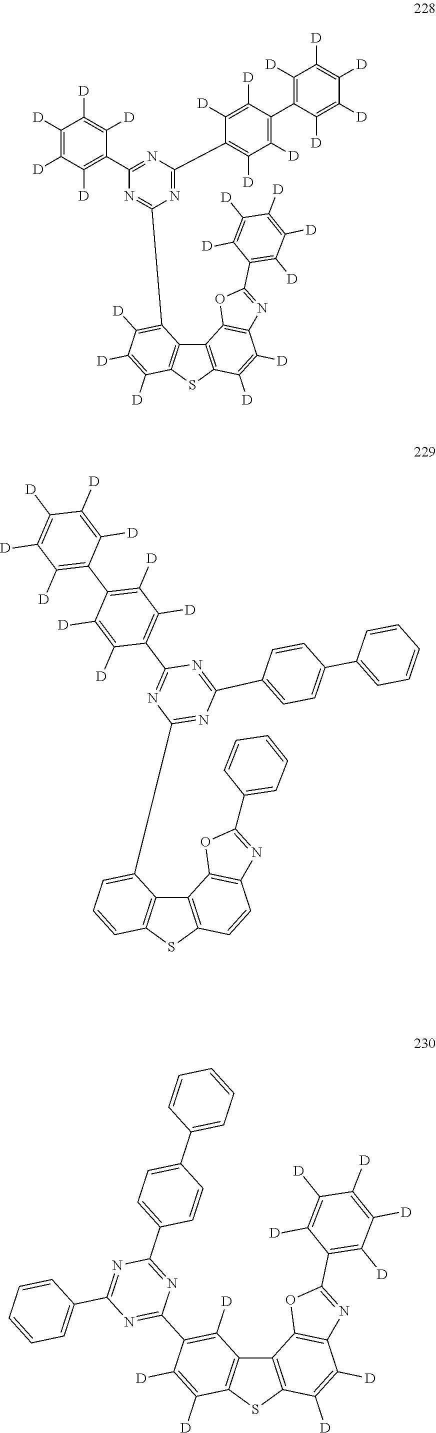 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c([2H])c4sc5c([2H])c([2H])c6nc(-c7c([2H])c([2H])c([2H])c([2H])c7[2H])oc6c5c34)n2)c([2H])c1[2H] ; [2H]c1c([2H])c([2H])c(-c2c([2H])c([2H])c(-c3nc(-c4ccc(-c5ccccc5)cc4)nc(-c4cccc5sc6ccc7nc(-c8ccccc8)oc7c6c45)n3)c([2H])c2[2H])c([2H])c1[2H] ; [2H]c1c([2H])c([2H])c(-c2nc3c([2H])c([2H])c4sc5c([2H])c([2H])c(-c6nc(-c7ccccc7)nc(-c7ccc(-c8ccccc8)cc7)n6)c([2H])c5c4c3o2)c([2H])c1[2H]]

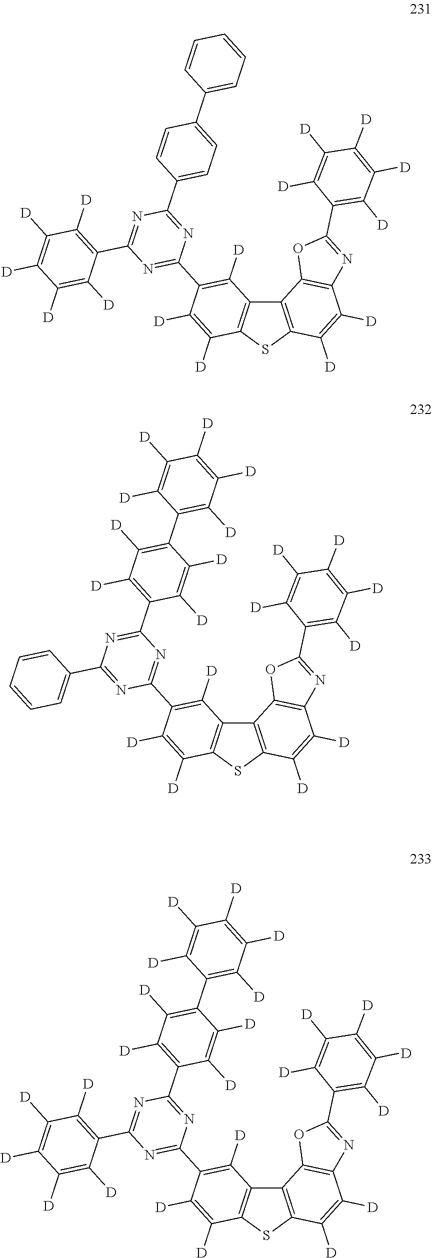

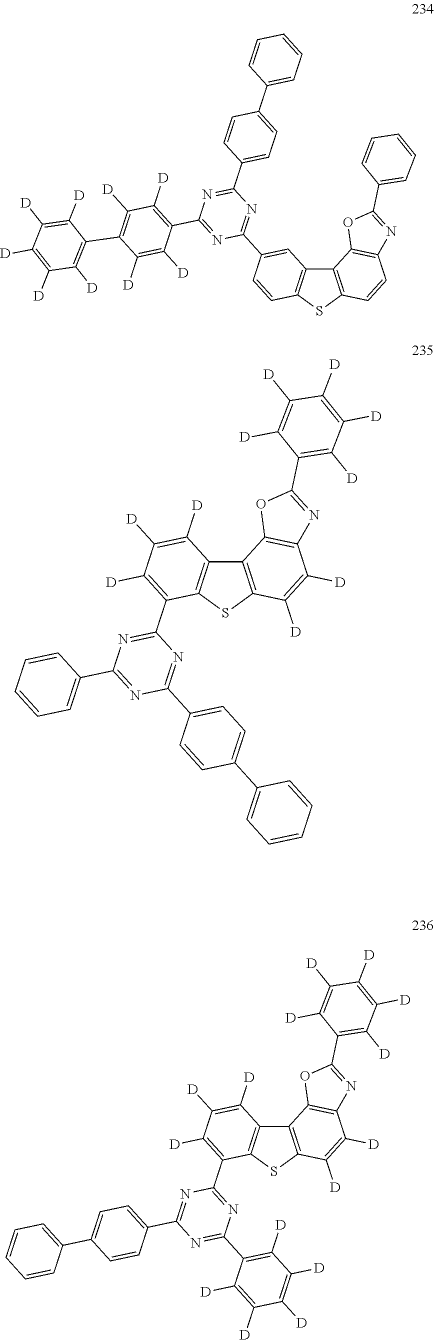 [SMILES: [2H]c1c([2H])c([2H])c(-c2c([2H])c([2H])c(-c3nc(-c4ccc(-c5ccccc5)cc4)nc(-c4ccc5sc6ccc7nc(-c8ccccc8)oc7c6c5c4)n3)c([2H])c2[2H])c([2H])c1[2H] ; [2H]c1c([2H])c([2H])c(-c2nc3c([2H])c([2H])c4sc5c(-c6nc(-c7ccccc7)nc(-c7ccc(-c8ccccc8)cc7)n6)c([2H])c([2H])c([2H])c5c4c3o2)c([2H])c1[2H] ; [2H]c1c([2H])c([2H])c(-c2nc(-c3ccc(-c4ccccc4)cc3)nc(-c3c([2H])c([2H])c([2H])c4c3sc3c([2H])c([2H])c5nc(-c6c([2H])c([2H])c([2H])c([2H])c6[2H])oc5c34)n2)c([2H])c1[2H]]

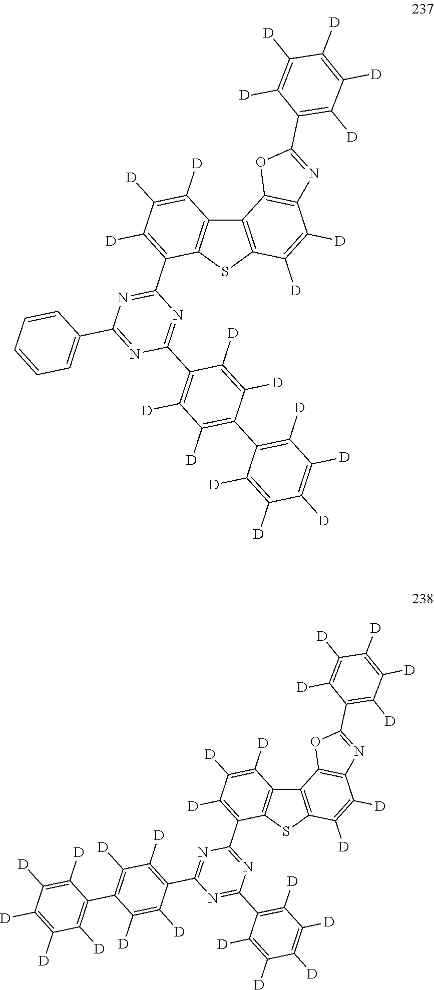 [SMILES: [2H]c1c([2H])c([2H])c(-c2nc3c([2H])c([2H])c4sc5c(-c6nc(-c7ccccc7)nc(-c7c([2H])c([2H])c(-c8c([2H])c([2H])c([2H])c([2H])c8[2H])c([2H])c7[2H])n6)c([2H])c([2H])c([2H])c5c4c3o2)c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c([2H])c4c3sc3c([2H])c([2H])c5nc(-c6c([2H])c([2H])c([2H])c([2H])c6[2H])oc5c34)n2)c([2H])c1[2H]]

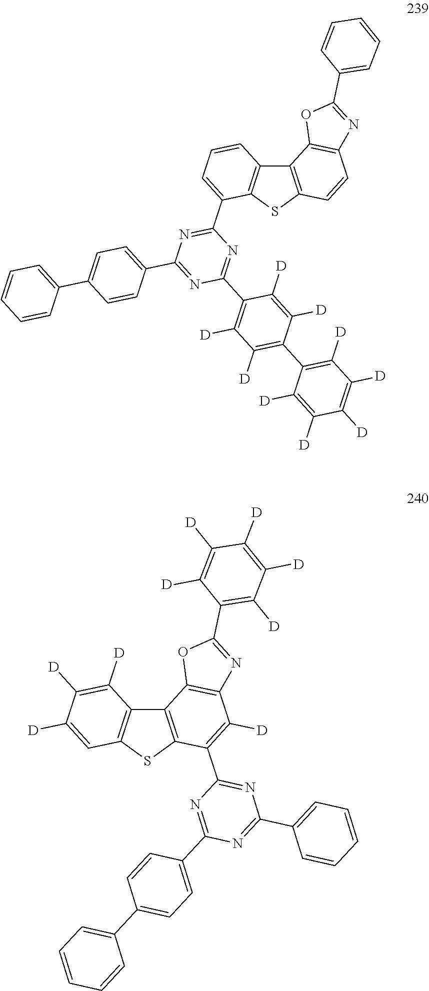 [SMILES: [2H]c1c([2H])c([2H])c(-c2c([2H])c([2H])c(-c3nc(-c4ccc(-c5ccccc5)cc4)nc(-c4cccc5c4sc4ccc6nc(-c7ccccc7)oc6c45)n3)c([2H])c2[2H])c([2H])c1[2H] ; [2H]c1cc2sc3c(-c4nc(-c5ccccc5)nc(-c5ccc(-c6ccccc6)cc5)n4)c([2H])c4nc(-c5c([2H])c([2H])c([2H])c([2H])c5[2H])oc4c3c2c([2H])c1[2H]]

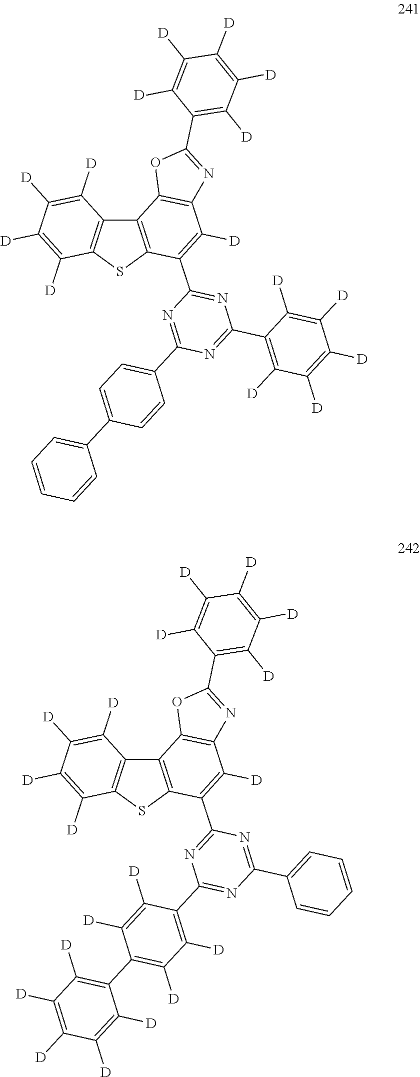 [SMILES: [2H]c1c([2H])c([2H])c(-c2nc(-c3ccc(-c4ccccc4)cc3)nc(-c3c([2H])c4nc(-c5c([2H])c([2H])c([2H])c([2H])c5[2H])oc4c4c3sc3c([2H])c([2H])c([2H])c([2H])c34)n2)c([2H])c1[2H] ; [2H]c1c([2H])c([2H])c(-c2nc3c([2H])c(-c4nc(-c5ccccc5)nc(-c5c([2H])c([2H])c(-c6c([2H])c([2H])c([2H])c([2H])c6[2H])c([2H])c5[2H])n4)c4sc5c([2H])c([2H])c([2H])c([2H])c5c4c3o2)c([2H])c1[2H]]

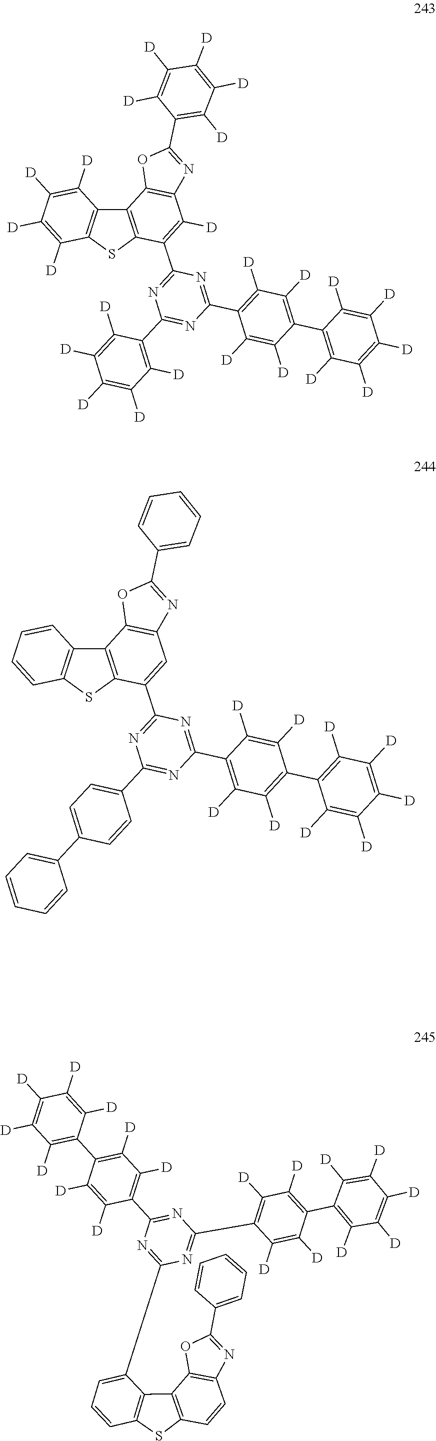

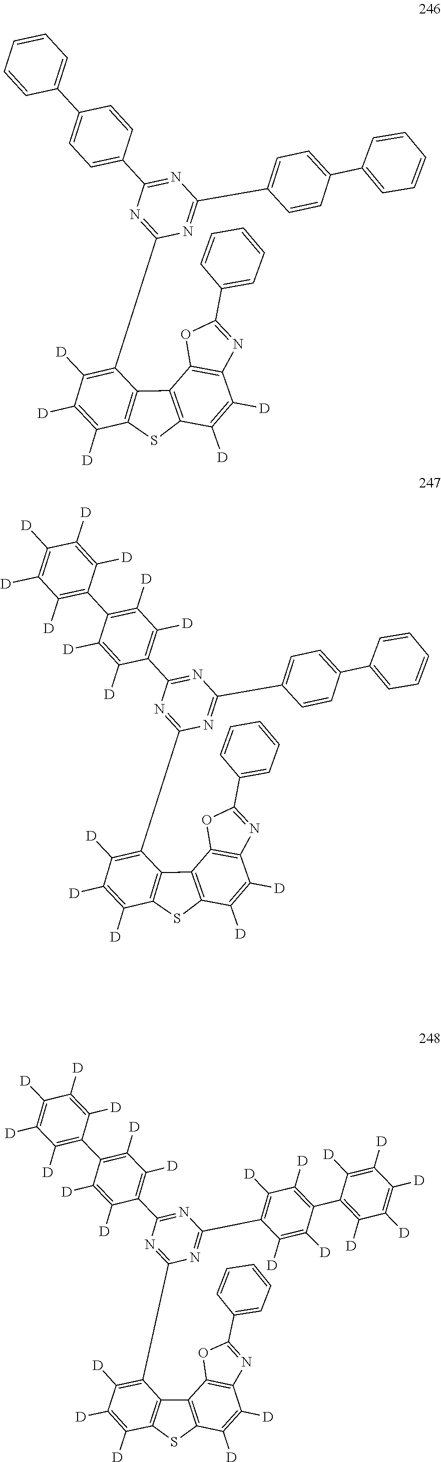

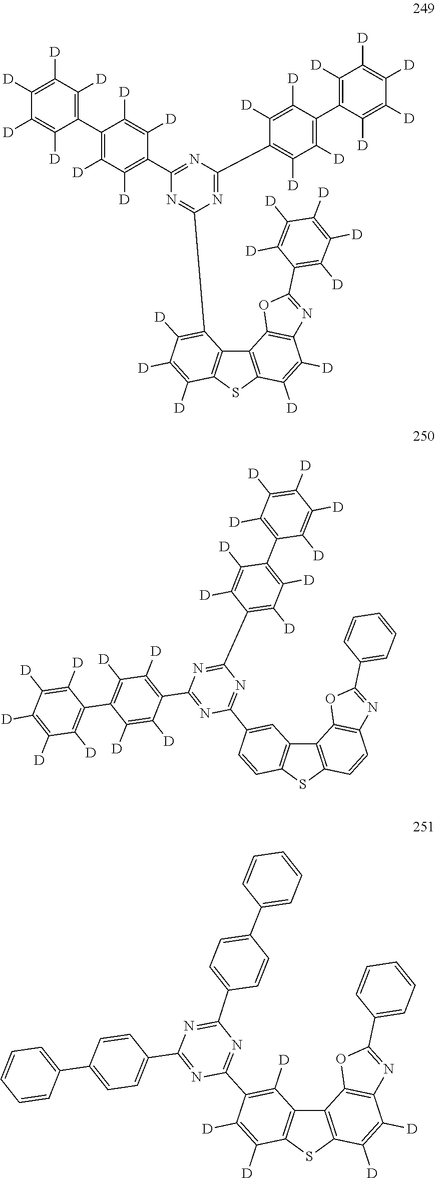

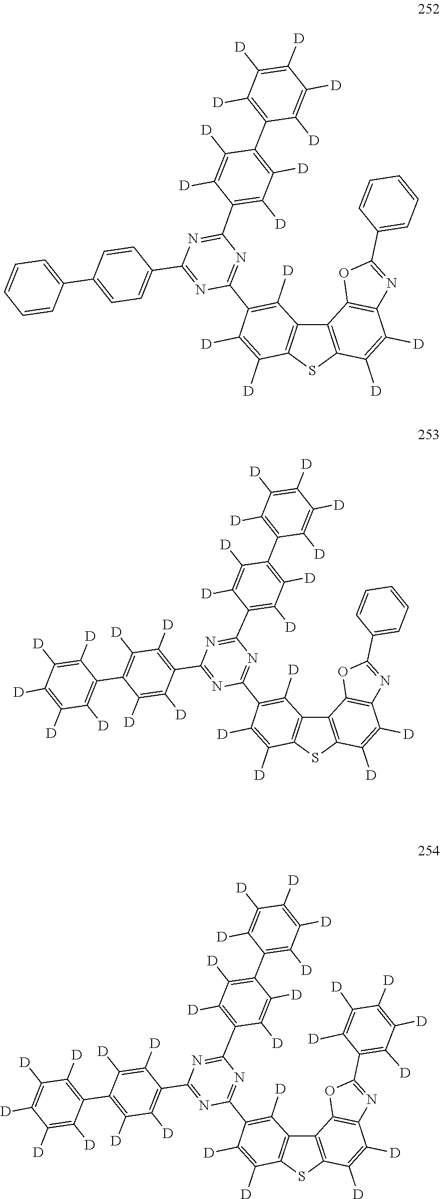

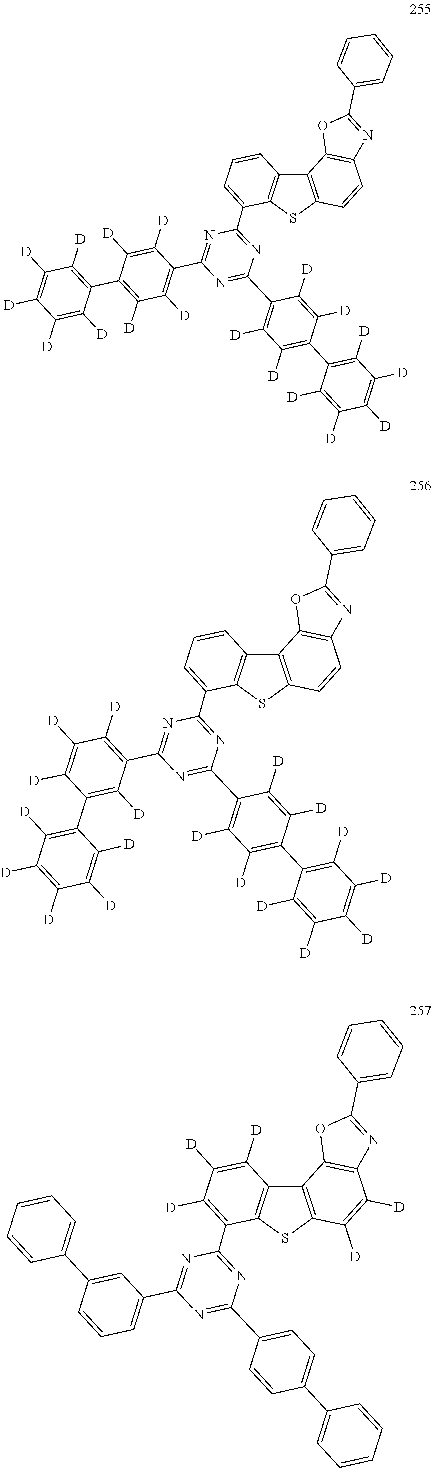 [SMILES: [2H]c1c([2H])c([2H])c(-c2c([2H])c([2H])c(-c3nc(-c4c([2H])c([2H])c(-c5c([2H])c([2H])c([2H])c([2H])c5[2H])c([2H])c4[2H])nc(-c4cccc5c4sc4ccc6nc(-c7ccccc7)oc6c45)n3)c([2H])c2[2H])c([2H])c1[2H] ; [2H]c1c([2H])c([2H])c(-c2c([2H])c([2H])c(-c3nc(-c4c([2H])c([2H])c([2H])c(-c5c([2H])c([2H])c([2H])c([2H])c5[2H])c4[2H])nc(-c4cccc5c4sc4ccc6nc(-c7ccccc7)oc6c45)n3)c([2H])c2[2H])c([2H])c1[2H] ; [2H]c1c([2H])c([2H])c2c(sc3c([2H])c([2H])c4nc(-c5ccccc5)oc4c32)c1-c1nc(-c2ccc(-c3ccccc3)cc2)nc(-c2cccc(-c3ccccc3)c2)n1]

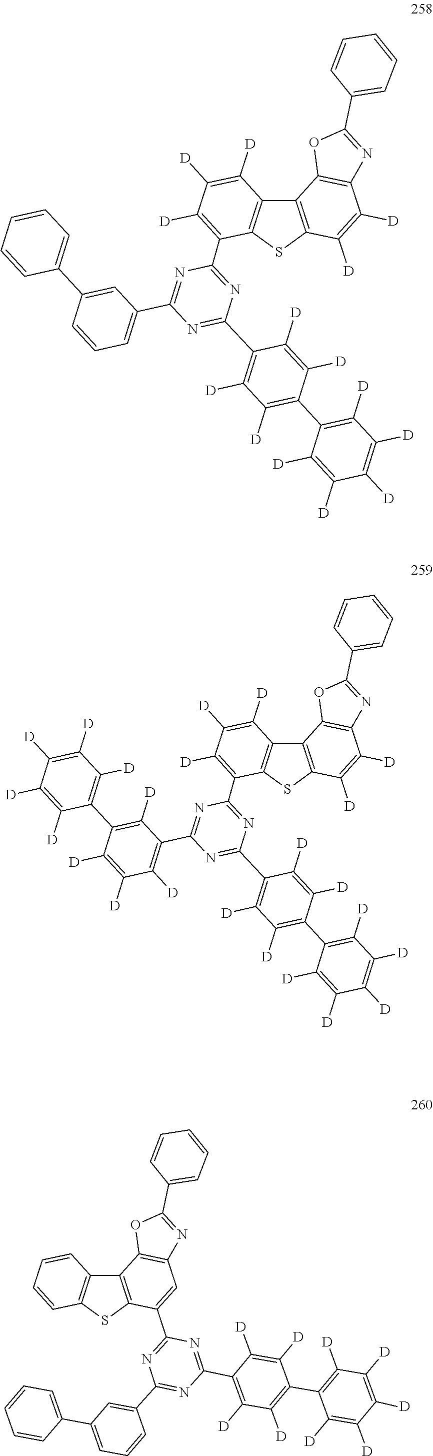

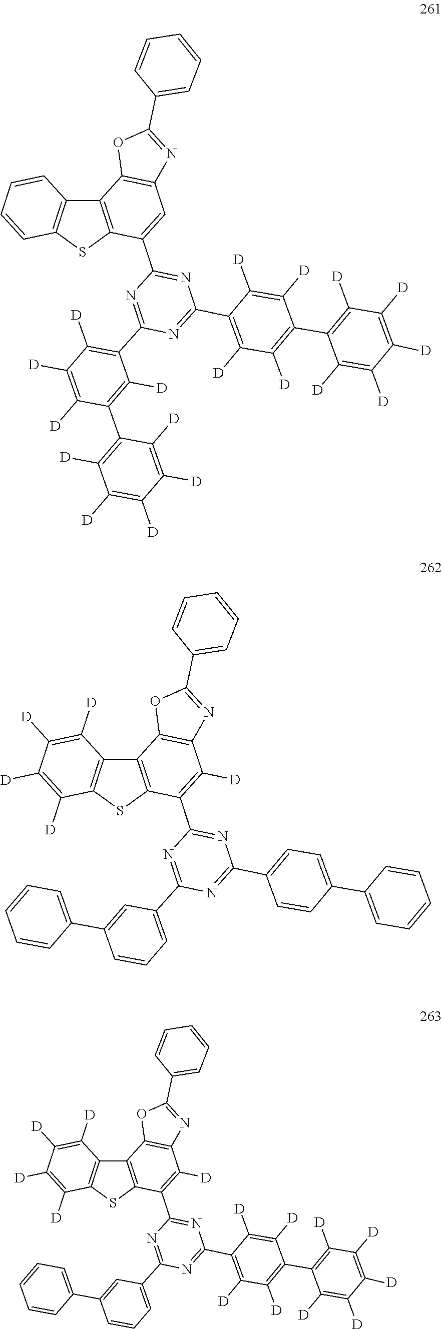 [SMILES: [2H]c1c([2H])c([2H])c(-c2c([2H])c([2H])c(-c3nc(-c4c([2H])c([2H])c([2H])c(-c5c([2H])c([2H])c([2H])c([2H])c5[2H])c4[2H])nc(-c4cc5nc(-c6ccccc6)oc5c5c4sc4ccccc45)n3)c([2H])c2[2H])c([2H])c1[2H] ; [2H]c1c([2H])c([2H])c2c(sc3c(-c4nc(-c5ccc(-c6ccccc6)cc5)nc(-c5cccc(-c6ccccc6)c5)n4)c([2H])c4nc(-c5ccccc5)oc4c32)c1[2H] ; [2H]c1c([2H])c([2H])c(-c2c([2H])c([2H])c(-c3nc(-c4cccc(-c5ccccc5)c4)nc(-c4c([2H])c5nc(-c6ccccc6)oc5c5c4sc4c([2H])c([2H])c([2H])c([2H])c45)n3)c([2H])c2[2H])c([2H])c1[2H]]

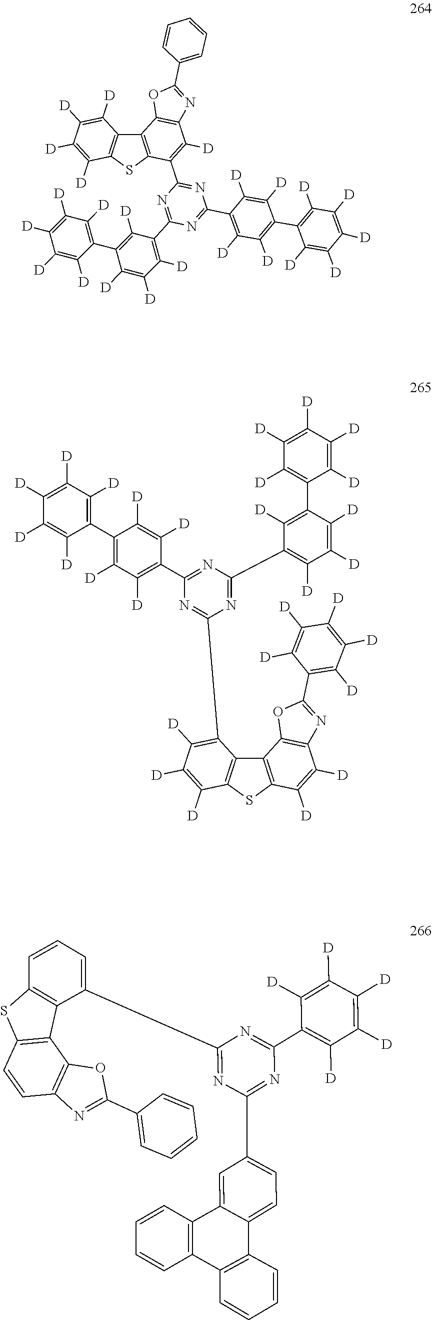

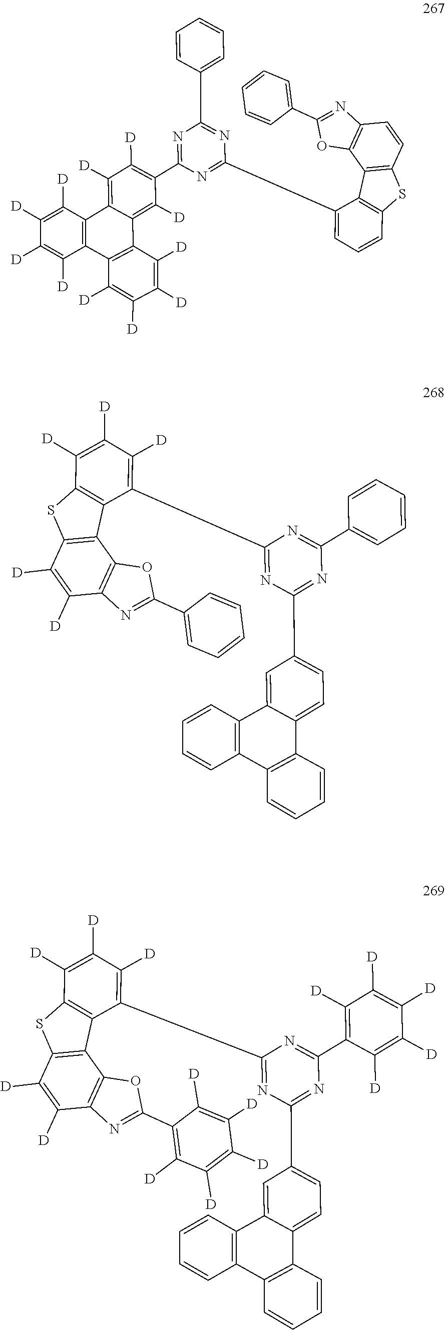 [SMILES: [2H]c1c([2H])c([2H])c2c(c1[2H])c1c([2H])c([2H])c([2H])c([2H])c1c1c([2H])c(-c3nc(-c4ccccc4)nc(-c4cccc5sc6ccc7nc(-c8ccccc8)oc7c6c45)n3)c([2H])c([2H])c21 ; [2H]c1c([2H])c(-c2nc(-c3ccccc3)nc(-c3ccc4c5ccccc5c5ccccc5c4c3)n2)c2c(sc3c([2H])c([2H])c4nc(-c5ccccc5)oc4c32)c1[2H] ; [2H]c1c([2H])c([2H])c(-c2nc(-c3ccc4c5ccccc5c5ccccc5c4c3)nc(-c3c([2H])c([2H])c([2H])c4sc5c([2H])c([2H])c6nc(-c7c([2H])c([2H])c([2H])c([2H])c7[2H])oc6c5c34)n2)c([2H])c1[2H]]

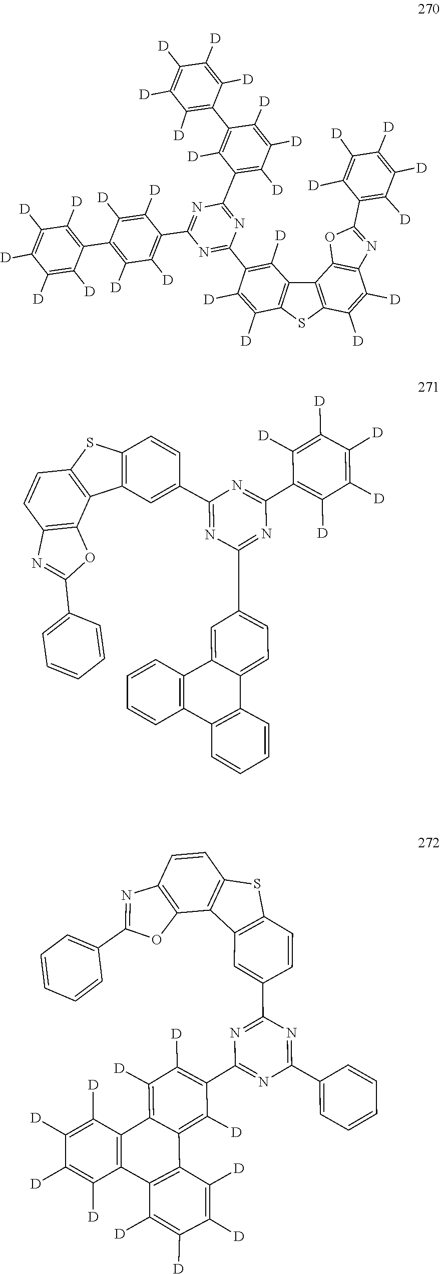

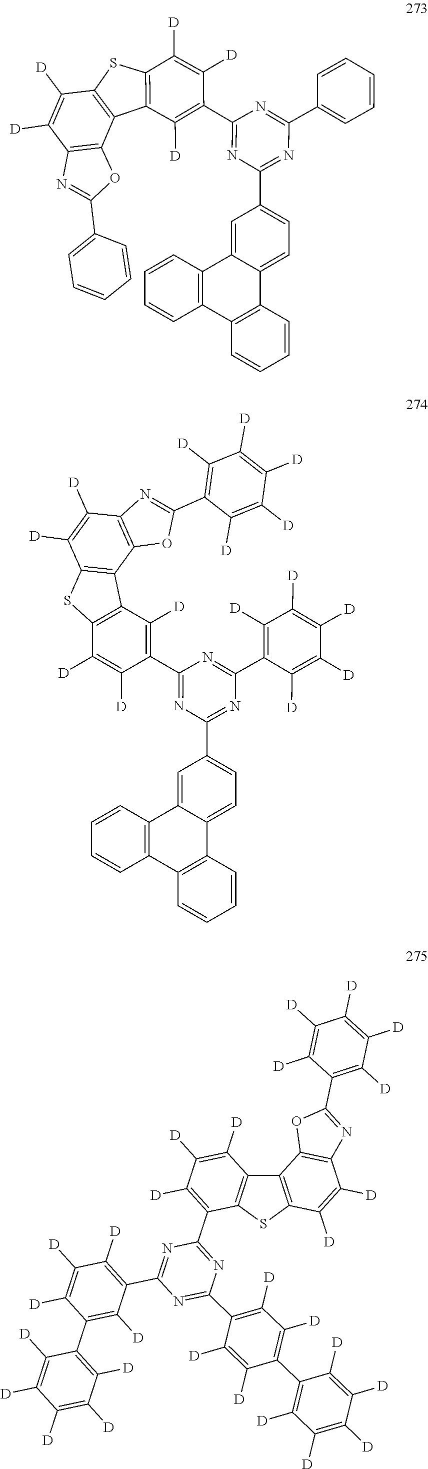

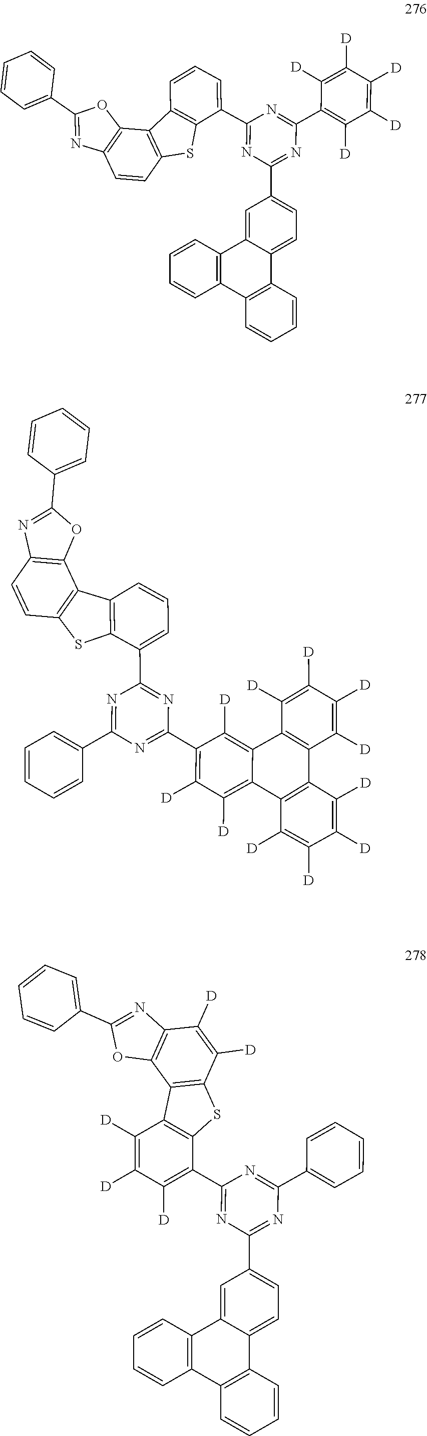 [SMILES: [2H]c1c([2H])c([2H])c(-c2nc(-c3ccc4c5ccccc5c5ccccc5c4c3)nc(-c3cccc4c3sc3ccc5nc(-c6ccccc6)oc5c34)n2)c([2H])c1[2H] ; [2H]c1c([2H])c([2H])c2c(c1[2H])c1c([2H])c([2H])c([2H])c([2H])c1c1c([2H])c(-c3nc(-c4ccccc4)nc(-c4cccc5c4sc4ccc6nc(-c7ccccc7)oc6c45)n3)c([2H])c([2H])c21 ; [2H]c1c([2H])c([2H])c2c(sc3c([2H])c([2H])c4nc(-c5ccccc5)oc4c32)c1-c1nc(-c2ccccc2)nc(-c2ccc3c4ccccc4c4ccccc4c3c2)n1]

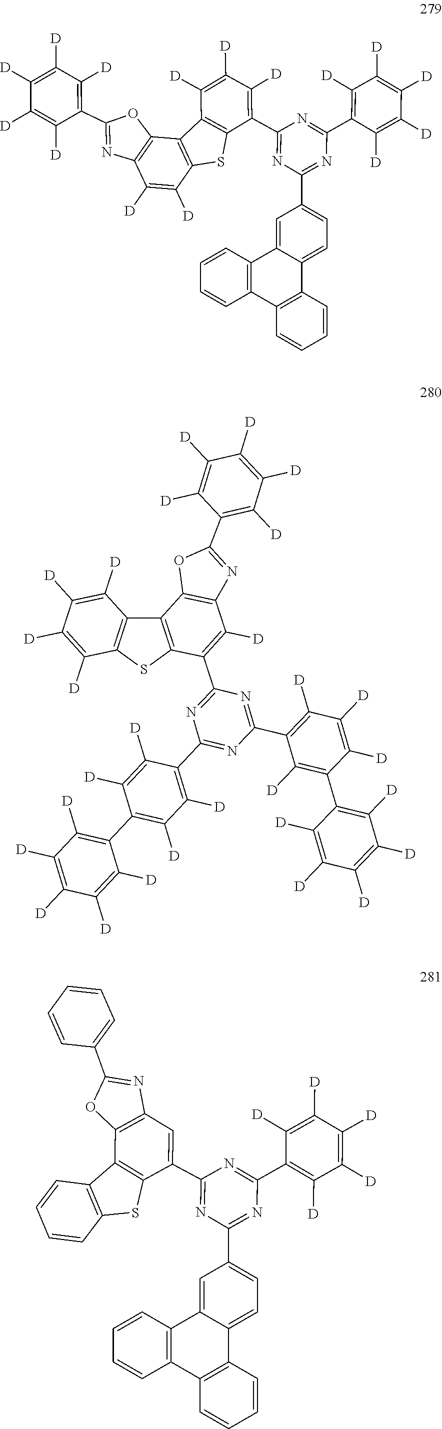

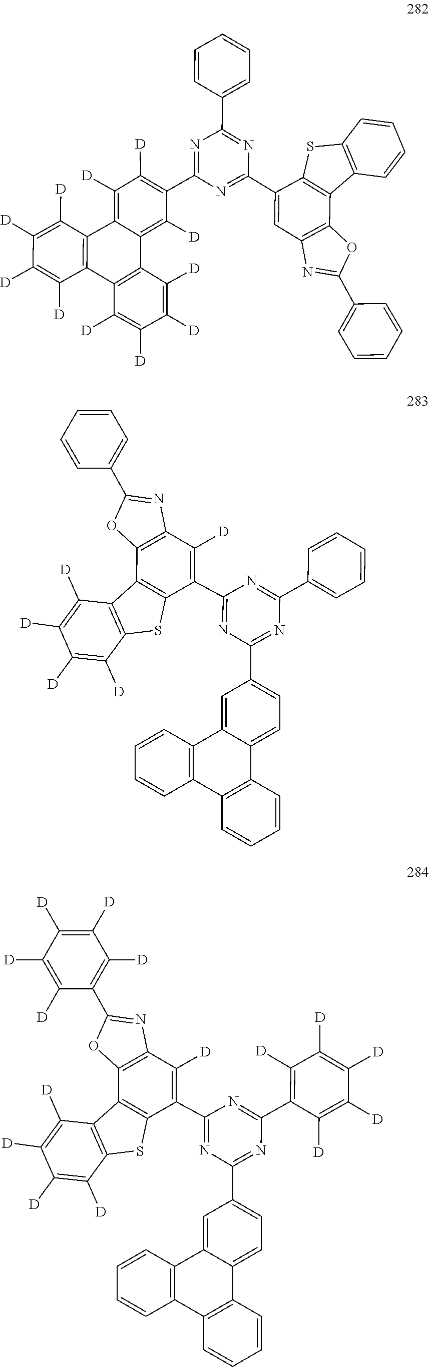 [SMILES: [2H]c1c([2H])c([2H])c2c(c1[2H])c1c([2H])c([2H])c([2H])c([2H])c1c1c([2H])c(-c3nc(-c4ccccc4)nc(-c4cc5nc(-c6ccccc6)oc5c5c4sc4ccccc45)n3)c([2H])c([2H])c21 ; [2H]c1c([2H])c([2H])c2c(sc3c(-c4nc(-c5ccccc5)nc(-c5ccc6c7ccccc7c7ccccc7c6c5)n4)c([2H])c4nc(-c5ccccc5)oc4c32)c1[2H] ; [2H]c1c([2H])c([2H])c(-c2nc(-c3ccc4c5ccccc5c5ccccc5c4c3)nc(-c3c([2H])c4nc(-c5c([2H])c([2H])c([2H])c([2H])c5[2H])oc4c4c3sc3c([2H])c([2H])c([2H])c([2H])c34)n2)c([2H])c1[2H]]

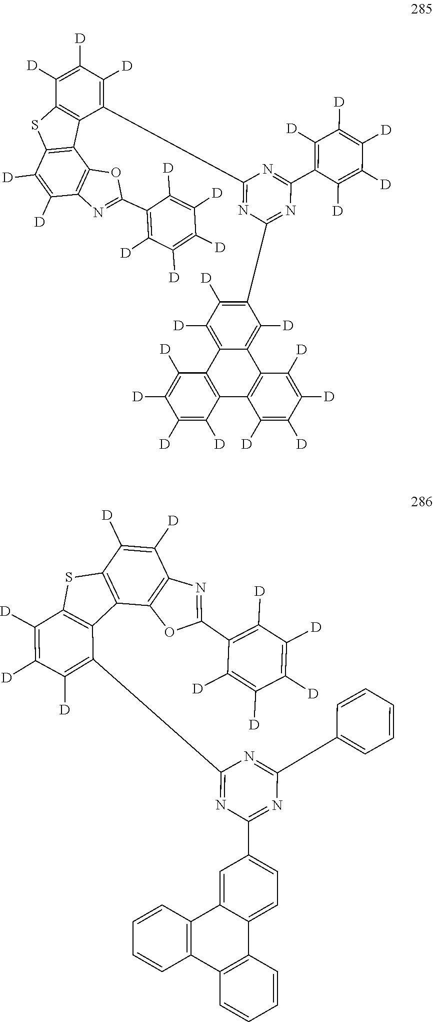 [SMILES: [2H]c1c([2H])c([2H])c(-c2nc(-c3c([2H])c([2H])c4c5c([2H])c([2H])c([2H])c([2H])c5c5c([2H])c([2H])c([2H])c([2H])c5c4c3[2H])nc(-c3c([2H])c([2H])c([2H])c4sc5c([2H])c([2H])c6nc(-c7c([2H])c([2H])c([2H])c([2H])c7[2H])oc6c5c34)n2)c([2H])c1[2H] ; [2H]c1c([2H])c([2H])c(-c2nc3c([2H])c([2H])c4sc5c([2H])c([2H])c([2H])c(-c6nc(-c7ccccc7)nc(-c7ccc8c9ccccc9c9ccccc9c8c7)n6)c5c4c3o2)c([2H])c1[2H]]

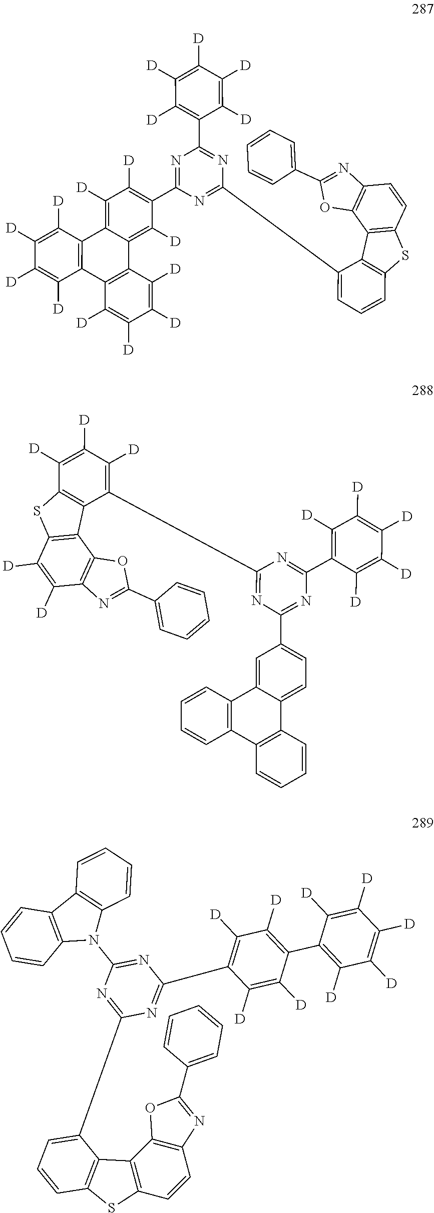 [SMILES: [2H]c1c([2H])c([2H])c(-c2nc(-c3c([2H])c([2H])c4c5c([2H])c([2H])c([2H])c([2H])c5c5c([2H])c([2H])c([2H])c([2H])c5c4c3[2H])nc(-c3cccc4sc5ccc6nc(-c7ccccc7)oc6c5c34)n2)c([2H])c1[2H] ; [2H]c1c([2H])c([2H])c(-c2nc(-c3ccc4c5ccccc5c5ccccc5c4c3)nc(-c3c([2H])c([2H])c([2H])c4sc5c([2H])c([2H])c6nc(-c7ccccc7)oc6c5c34)n2)c([2H])c1[2H] ; [2H]c1c([2H])c([2H])c(-c2c([2H])c([2H])c(-c3nc(-c4cccc5sc6ccc7nc(-c8ccccc8)oc7c6c45)nc(-n4c5ccccc5c5ccccc54)n3)c([2H])c2[2H])c([2H])c1[2H]]

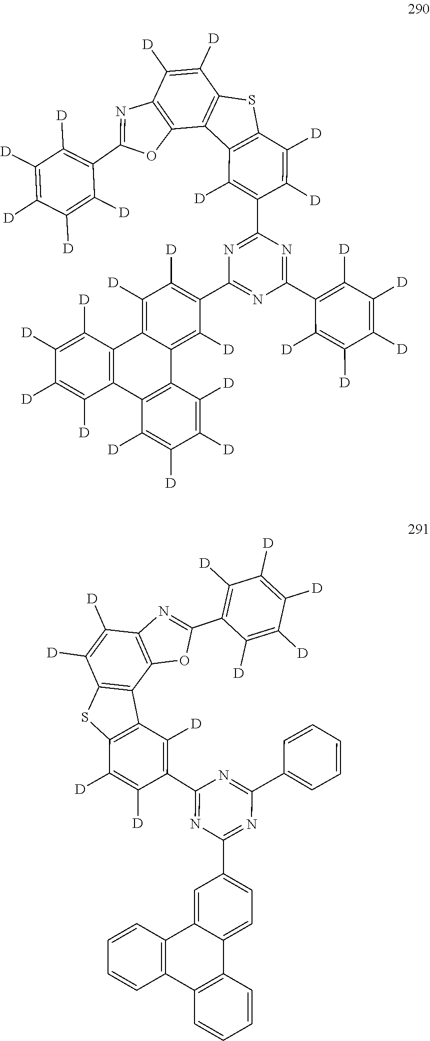 [SMILES: [2H]c1c([2H])c([2H])c(-c2nc(-c3c([2H])c([2H])c4c5c([2H])c([2H])c([2H])c([2H])c5c5c([2H])c([2H])c([2H])c([2H])c5c4c3[2H])nc(-c3c([2H])c([2H])c4sc5c([2H])c([2H])c6nc(-c7c([2H])c([2H])c([2H])c([2H])c7[2H])oc6c5c4c3[2H])n2)c([2H])c1[2H] ; [2H]c1c([2H])c([2H])c(-c2nc3c([2H])c([2H])c4sc5c([2H])c([2H])c(-c6nc(-c7ccccc7)nc(-c7ccc8c9ccccc9c9ccccc9c8c7)n6)c([2H])c5c4c3o2)c([2H])c1[2H]]

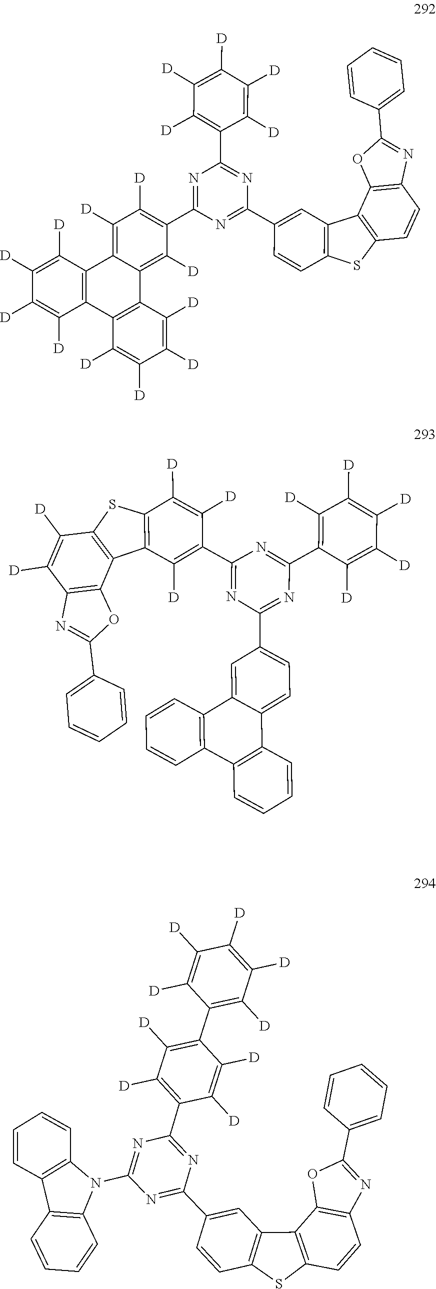 [SMILES: [2H]c1c([2H])c([2H])c(-c2nc(-c3ccc4sc5ccc6nc(-c7ccccc7)oc6c5c4c3)nc(-c3c([2H])c([2H])c4c5c([2H])c([2H])c([2H])c([2H])c5c5c([2H])c([2H])c([2H])c([2H])c5c4c3[2H])n2)c([2H])c1[2H] ; [2H]c1c([2H])c([2H])c(-c2nc(-c3ccc4c5ccccc5c5ccccc5c4c3)nc(-c3c([2H])c([2H])c4sc5c([2H])c([2H])c6nc(-c7ccccc7)oc6c5c4c3[2H])n2)c([2H])c1[2H] ; [2H]c1c([2H])c([2H])c(-c2c([2H])c([2H])c(-c3nc(-c4ccc5sc6ccc7nc(-c8ccccc8)oc7c6c5c4)nc(-n4c5ccccc5c5ccccc54)n3)c([2H])c2[2H])c([2H])c1[2H]]

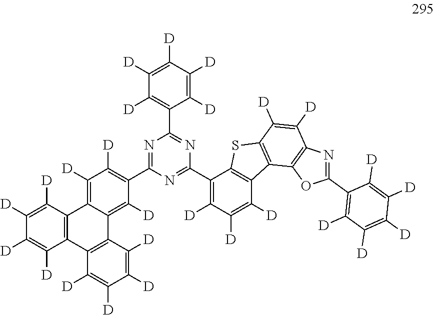 [SMILES: [2H]c1c([2H])c([2H])c(-c2nc(-c3c([2H])c([2H])c4c5c([2H])c([2H])c([2H])c([2H])c5c5c([2H])c([2H])c([2H])c([2H])c5c4c3[2H])nc(-c3c([2H])c([2H])c([2H])c4c3sc3c([2H])c([2H])c5nc(-c6c([2H])c([2H])c([2H])c([2H])c6[2H])oc5c34)n2)c([2H])c1[2H]]

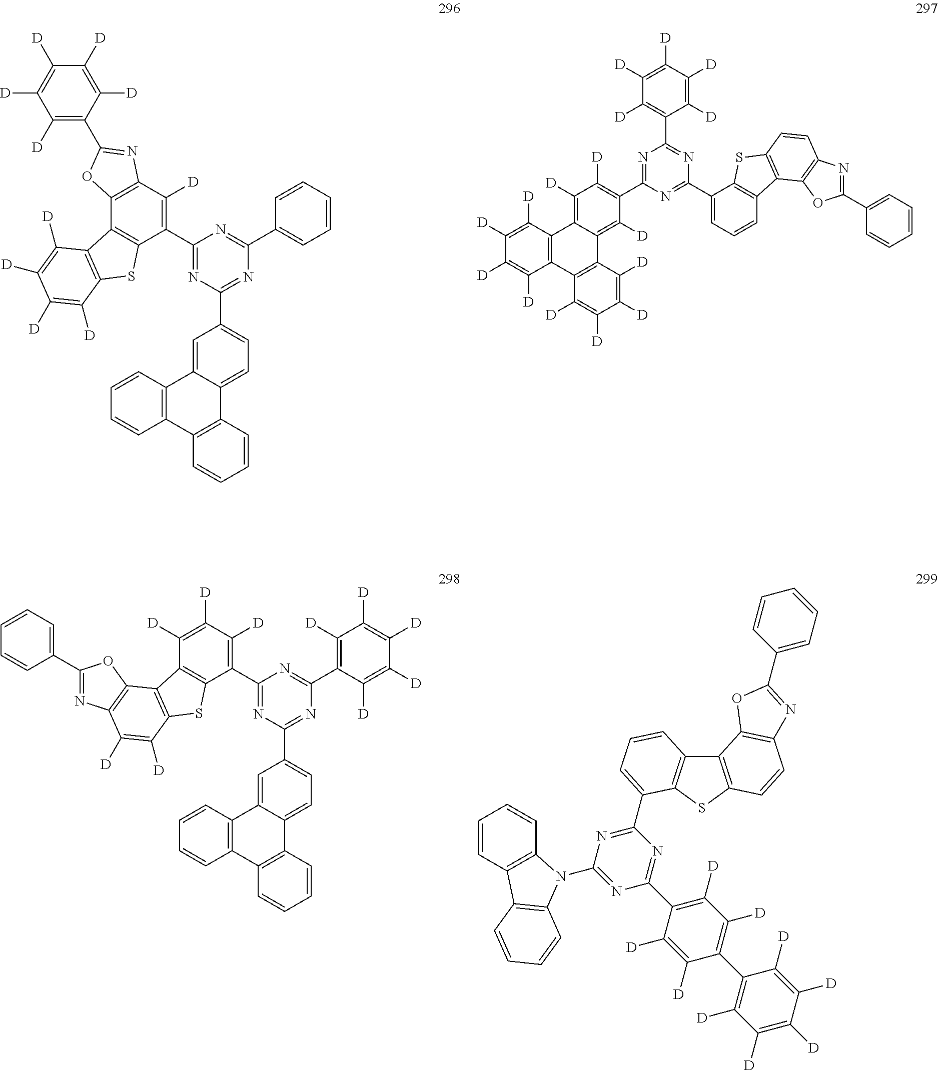

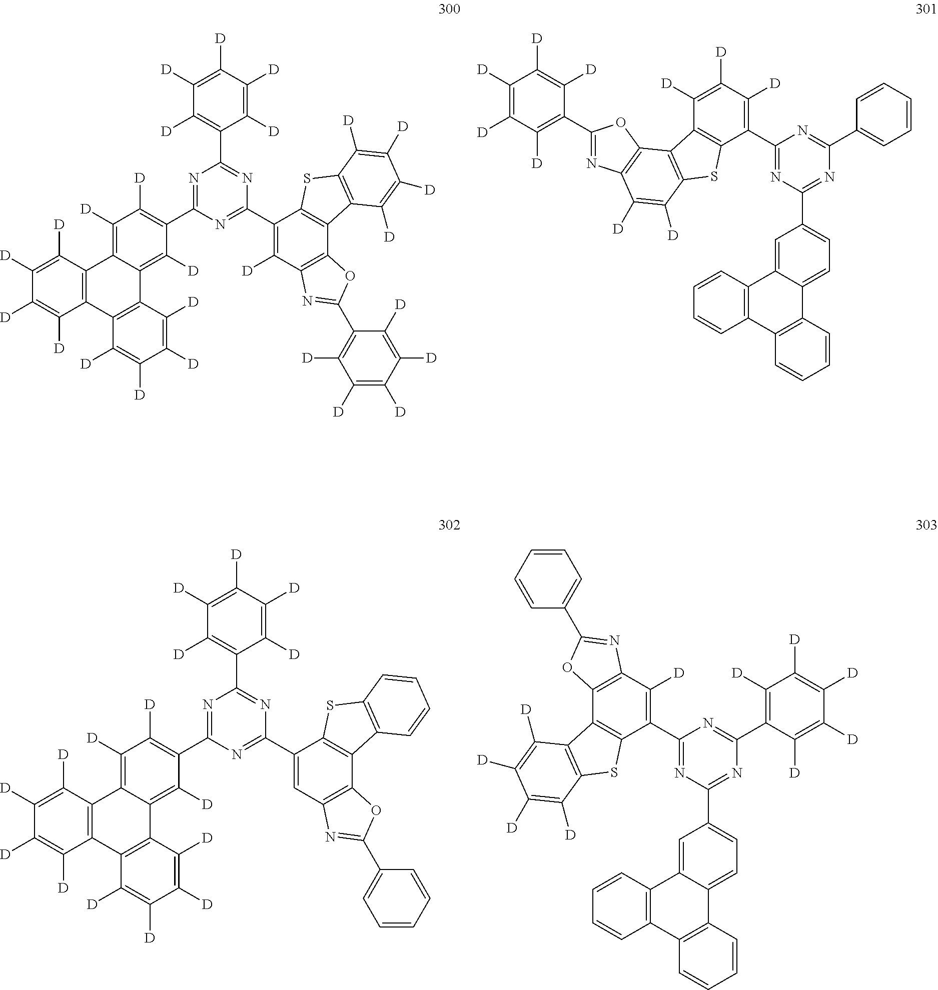

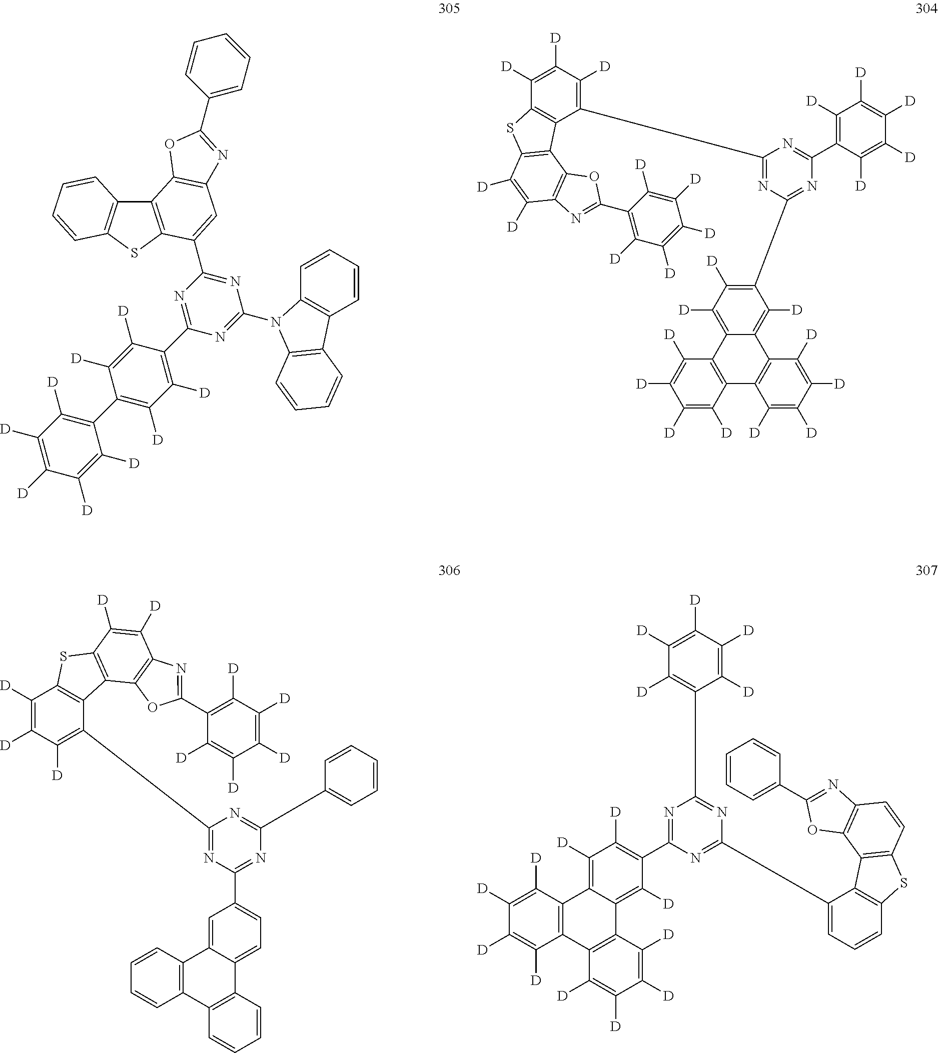

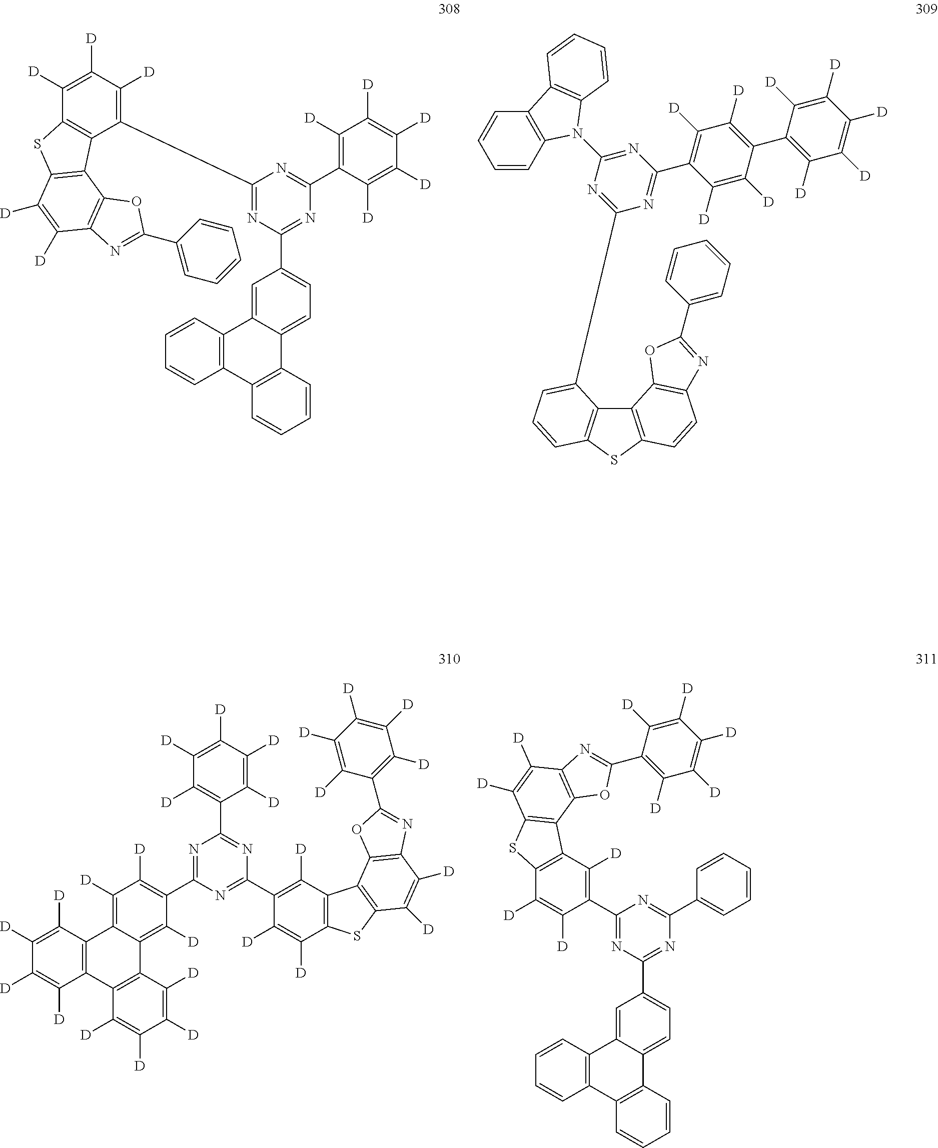

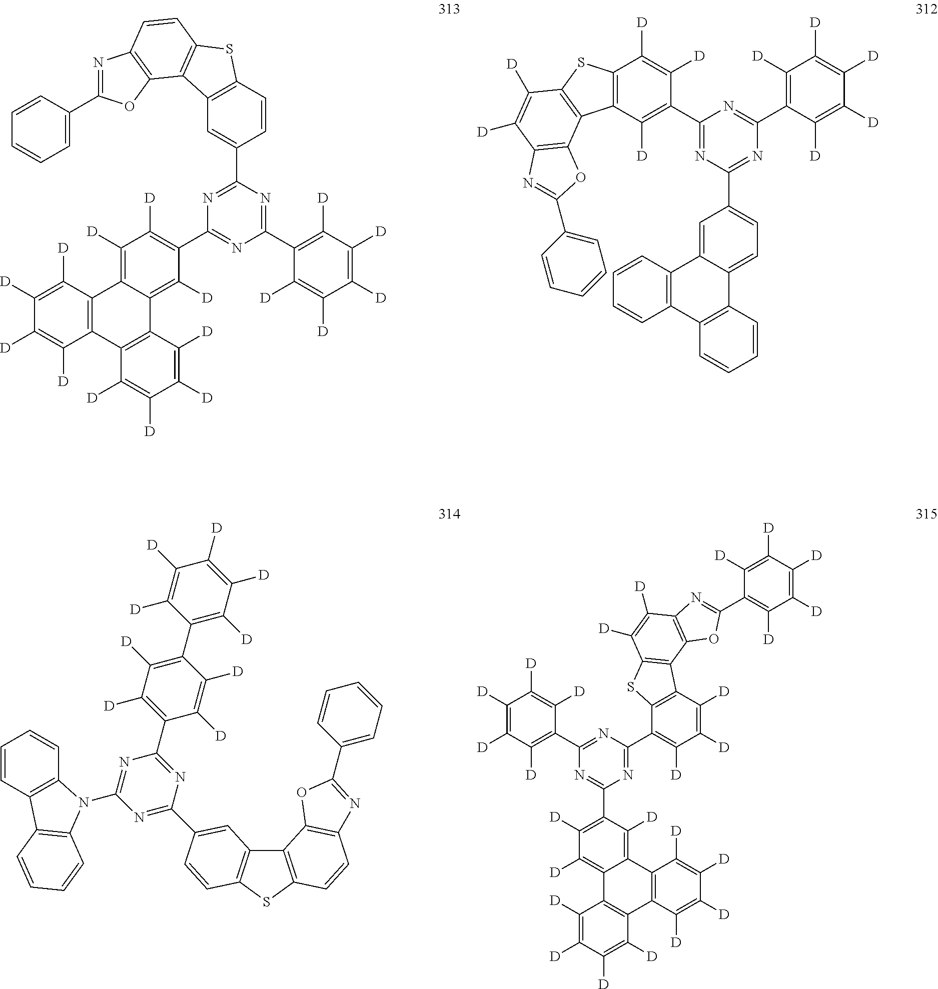

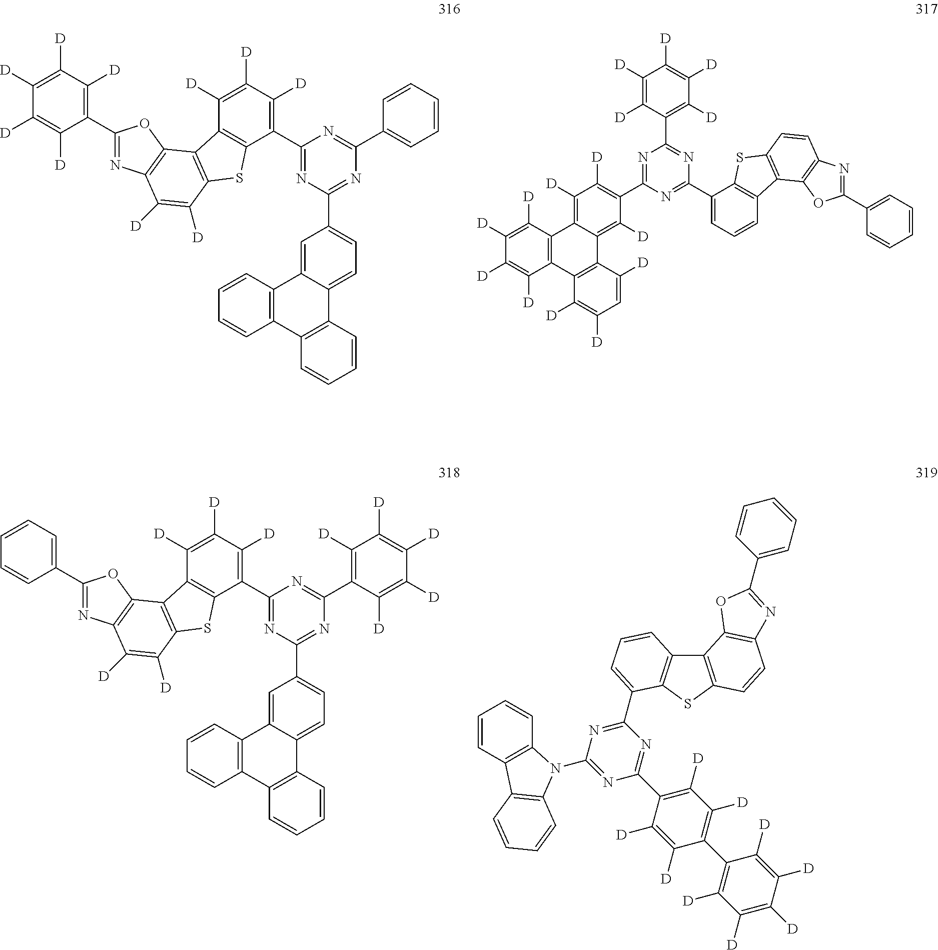

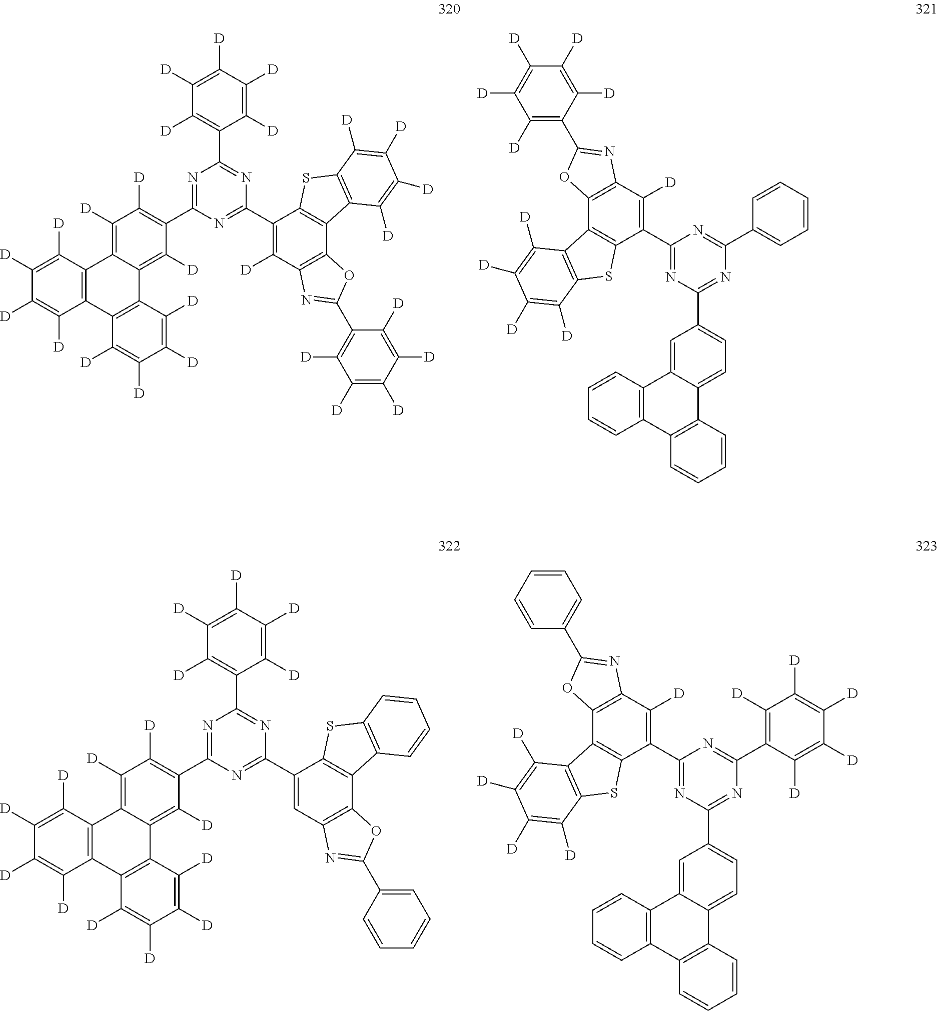

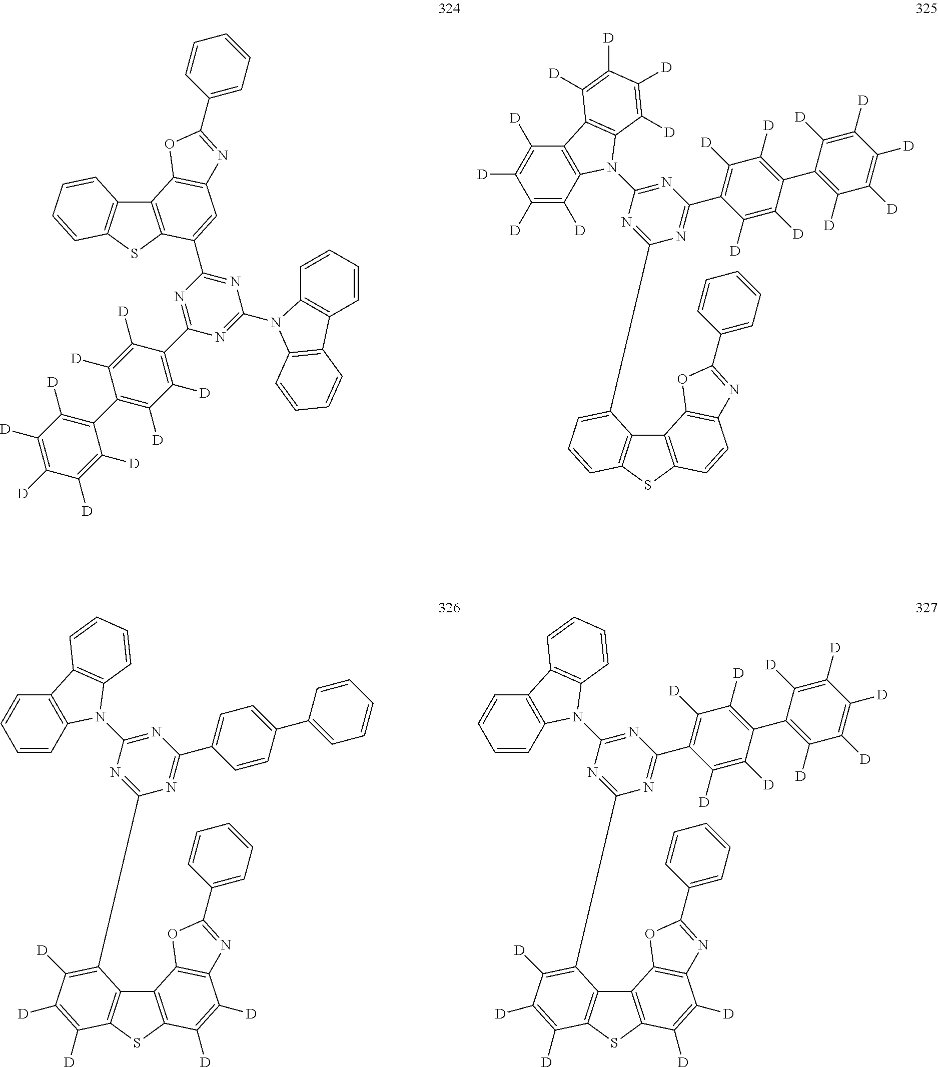

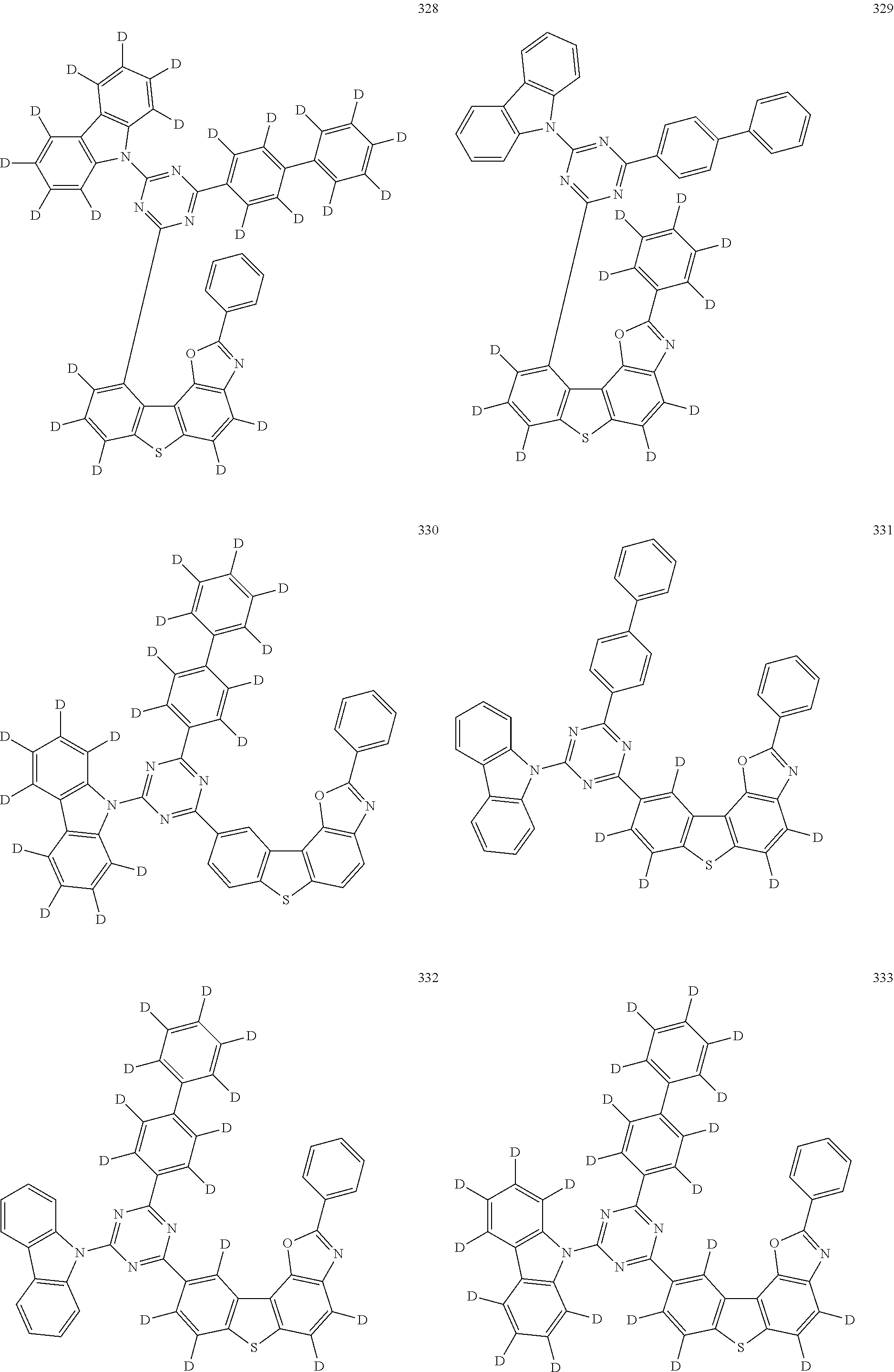

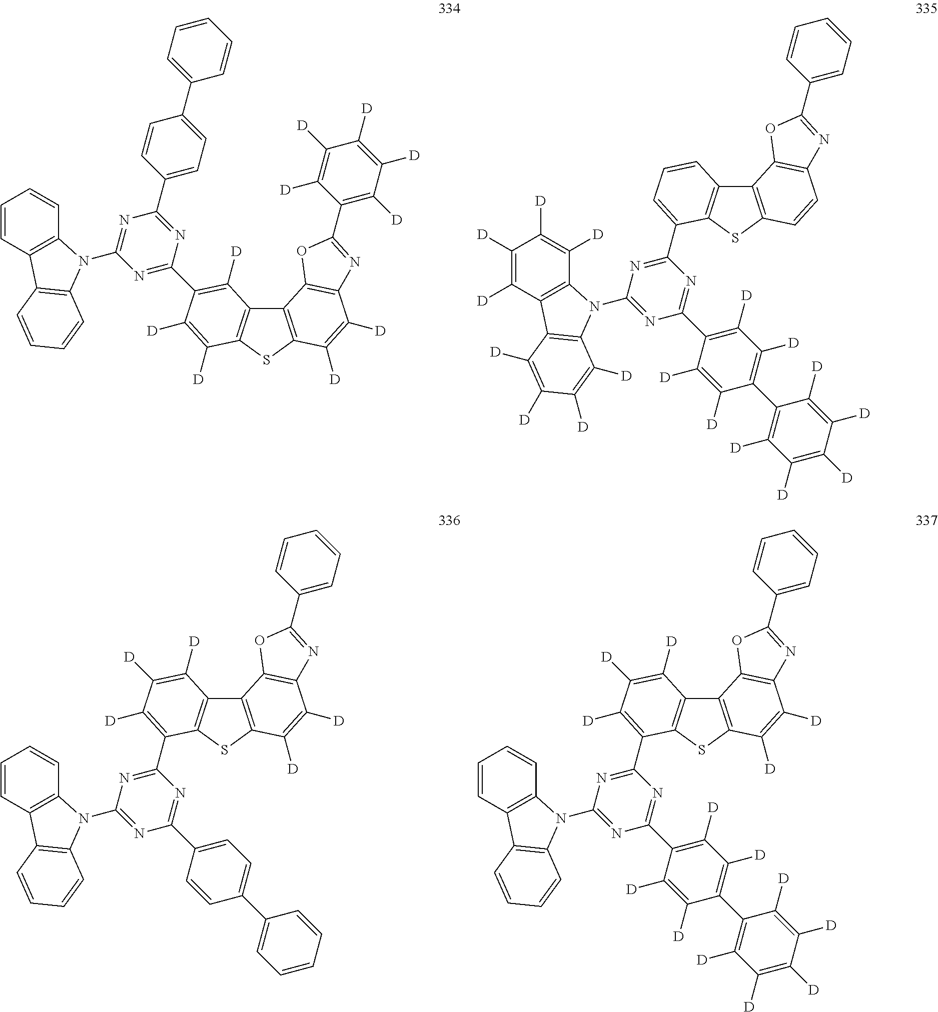

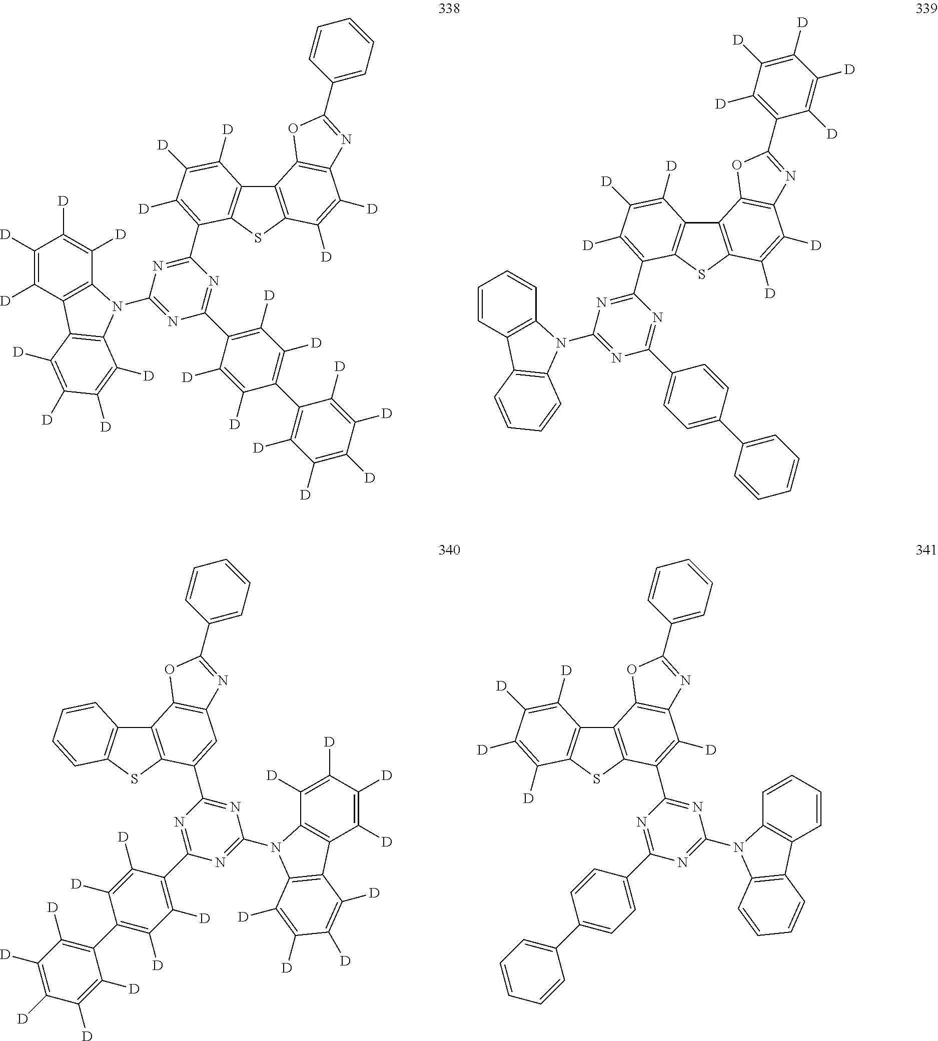

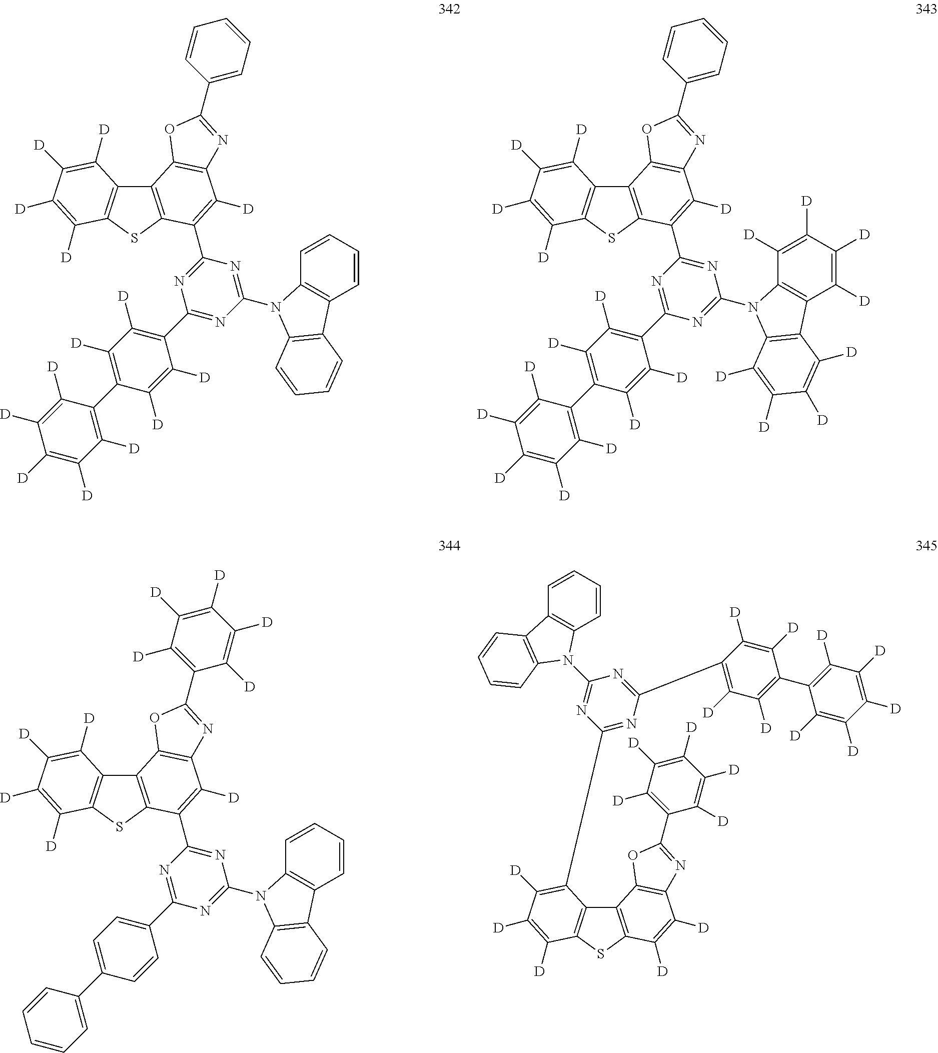

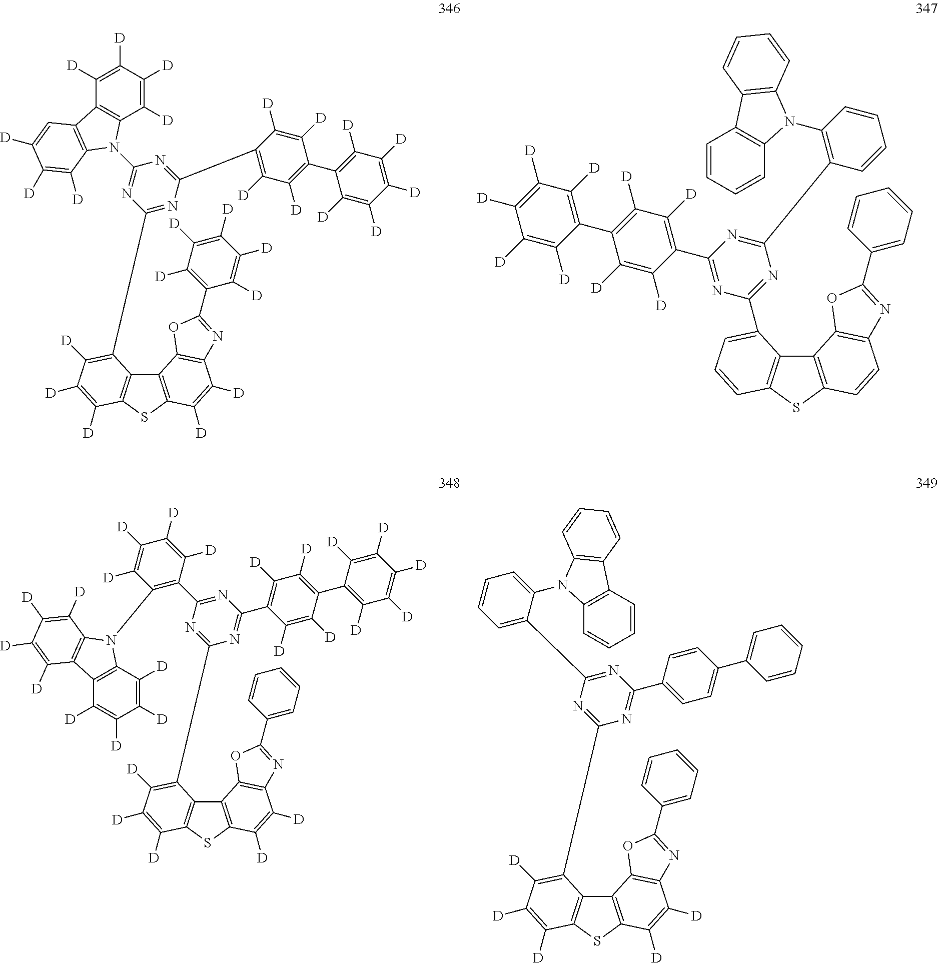

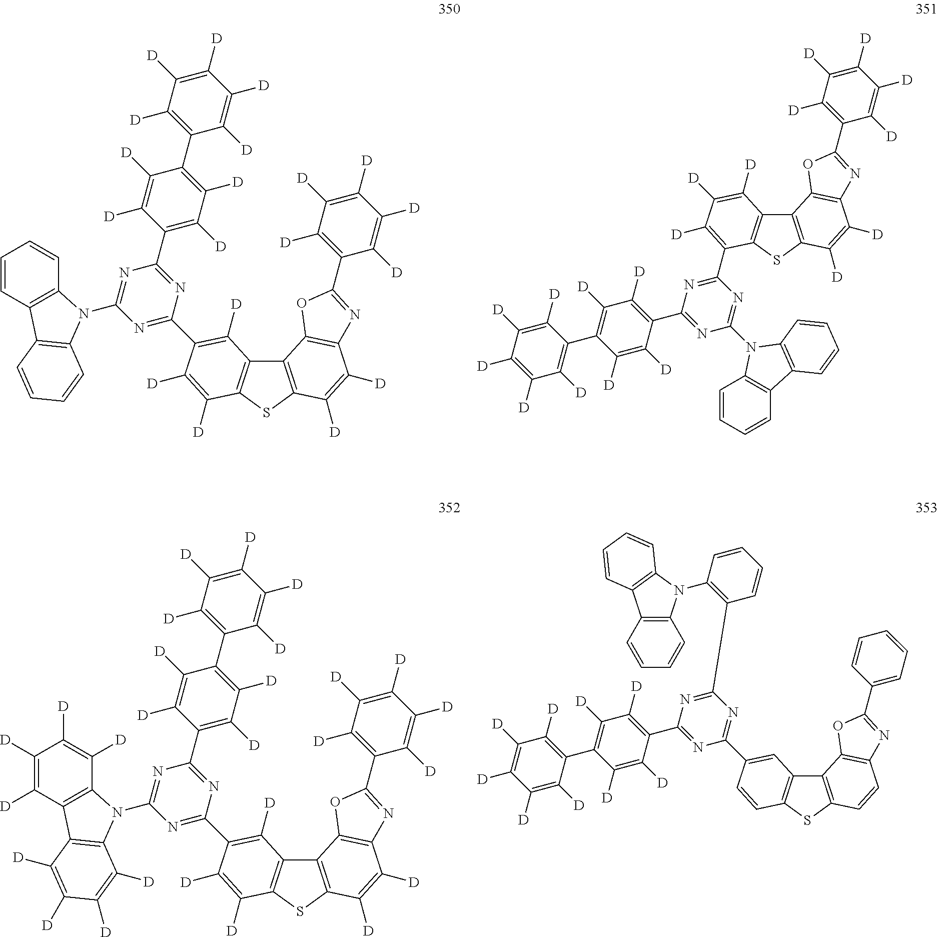

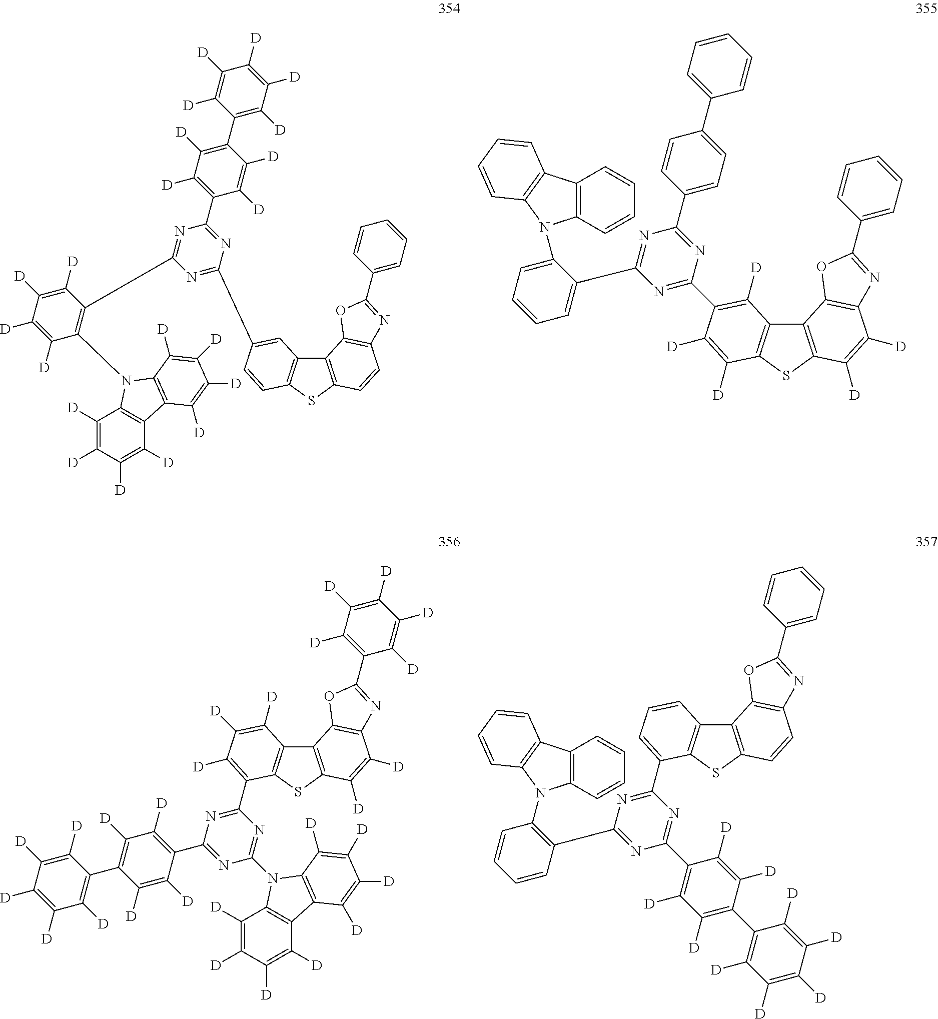

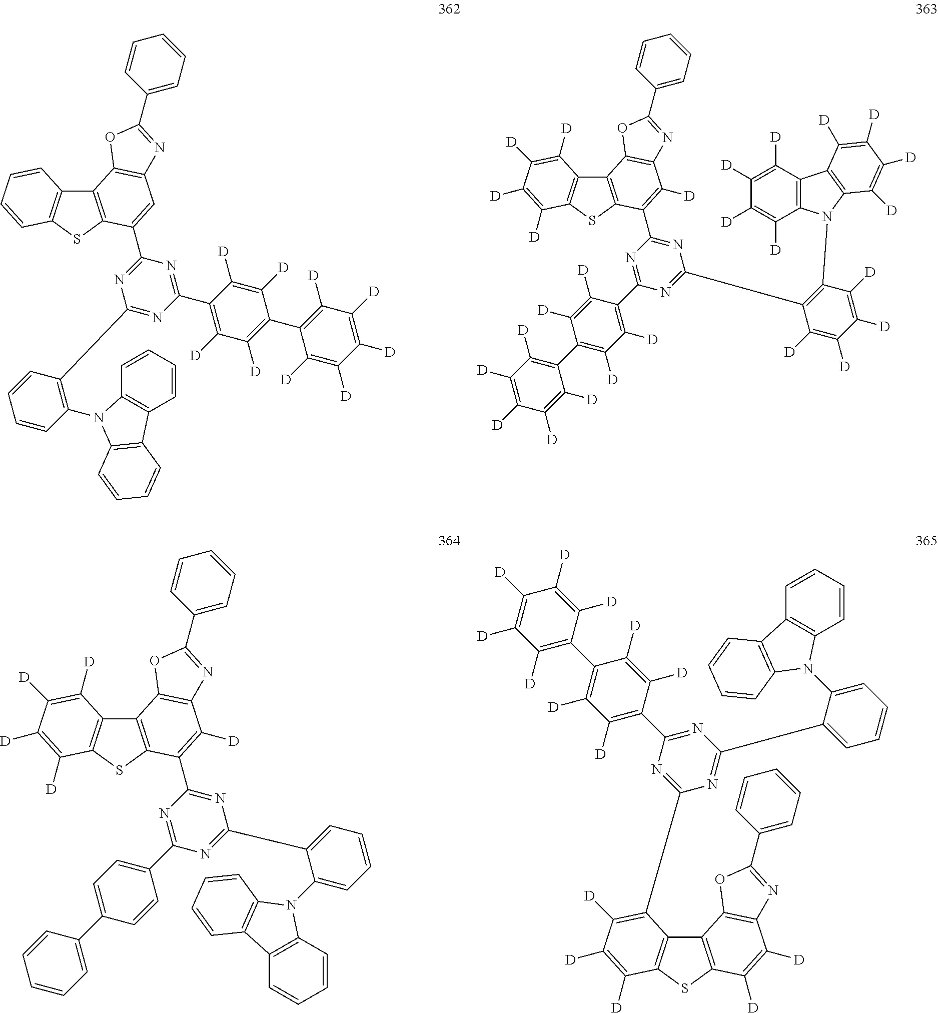

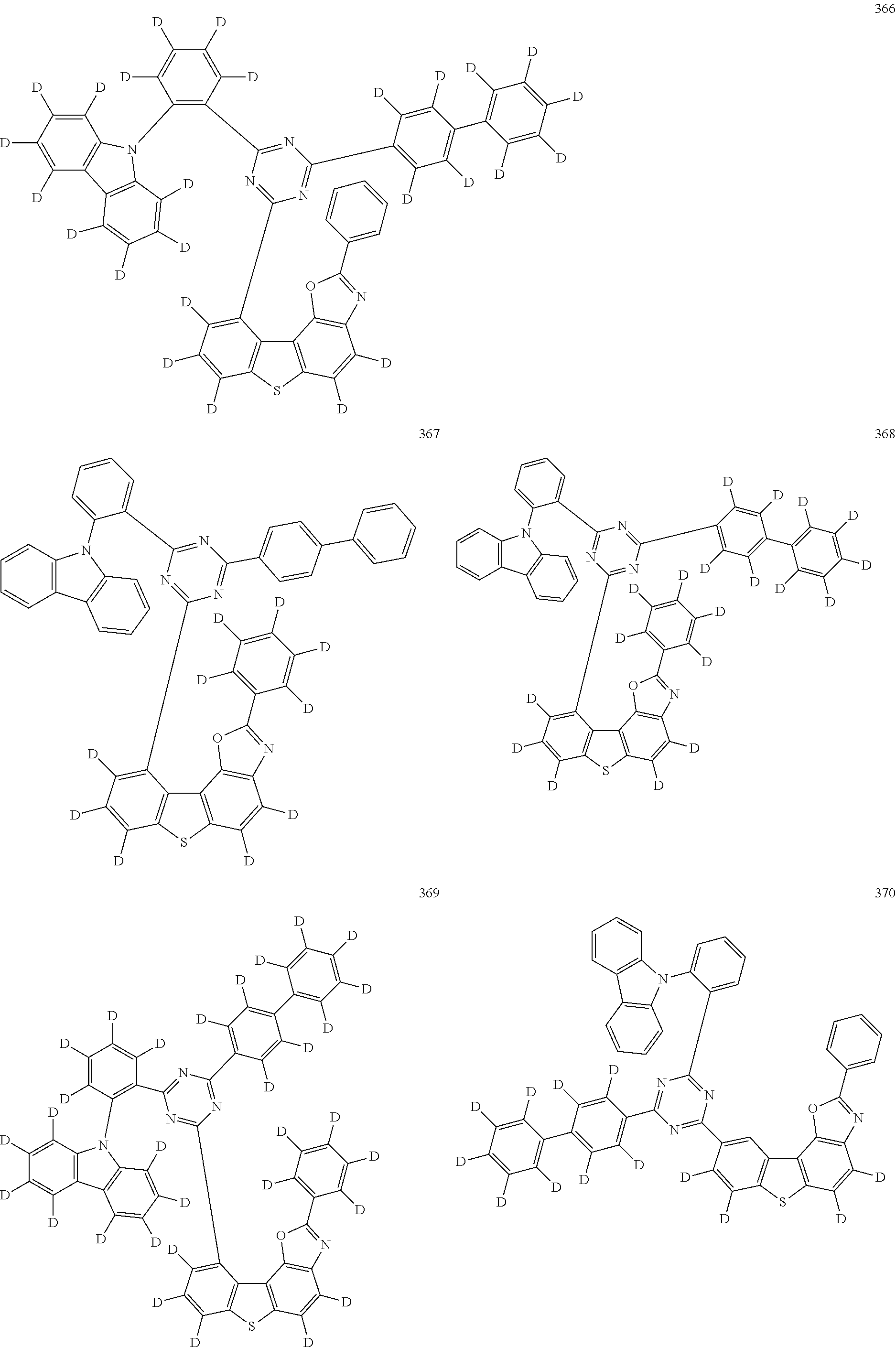

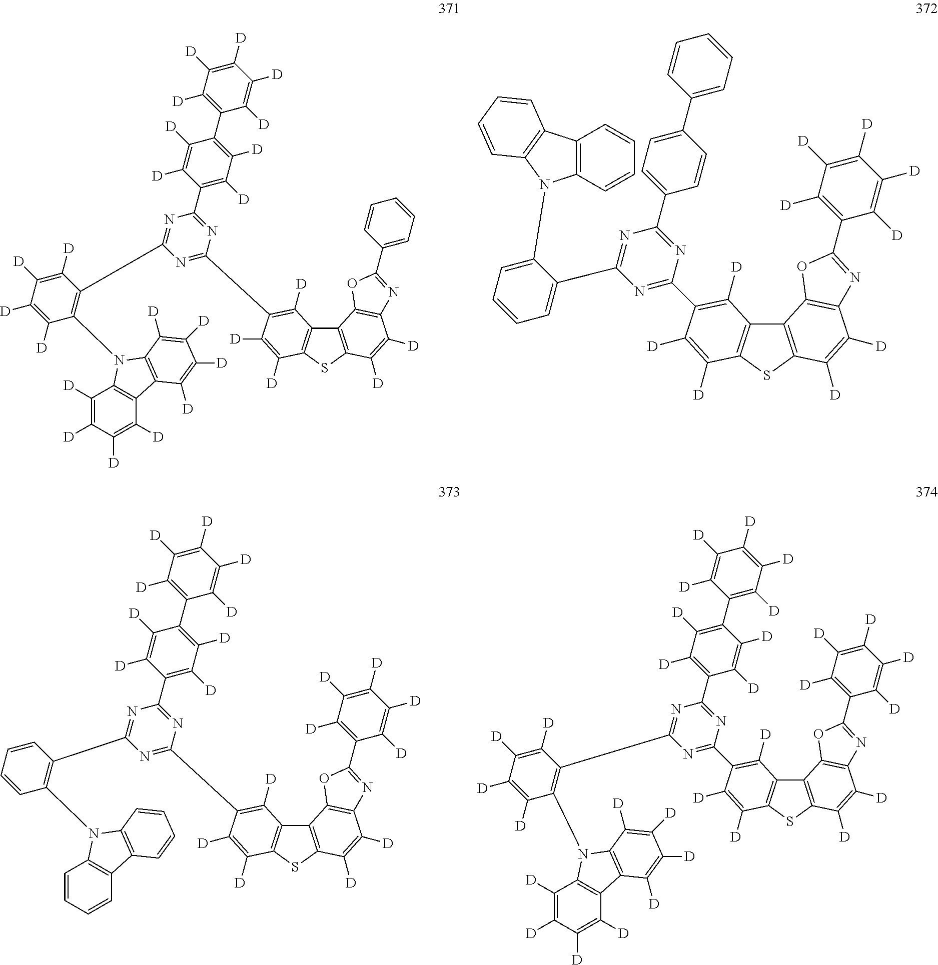

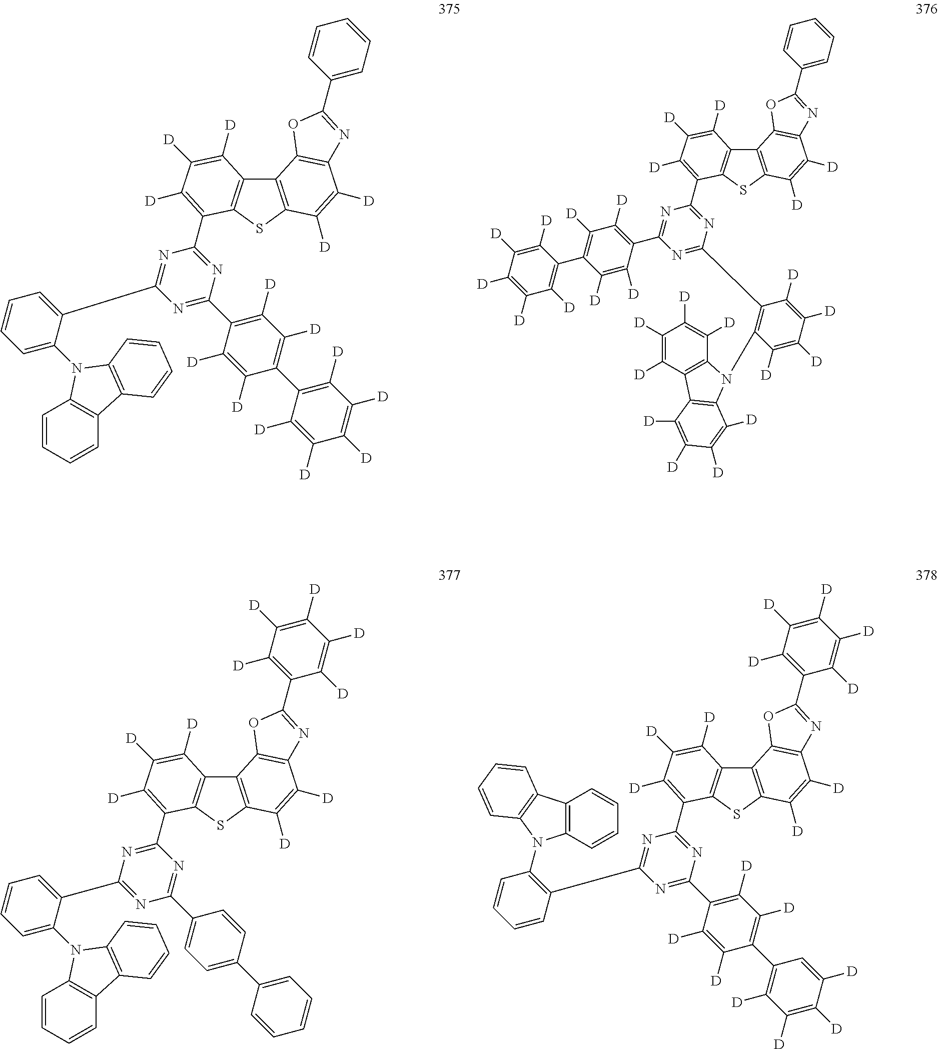

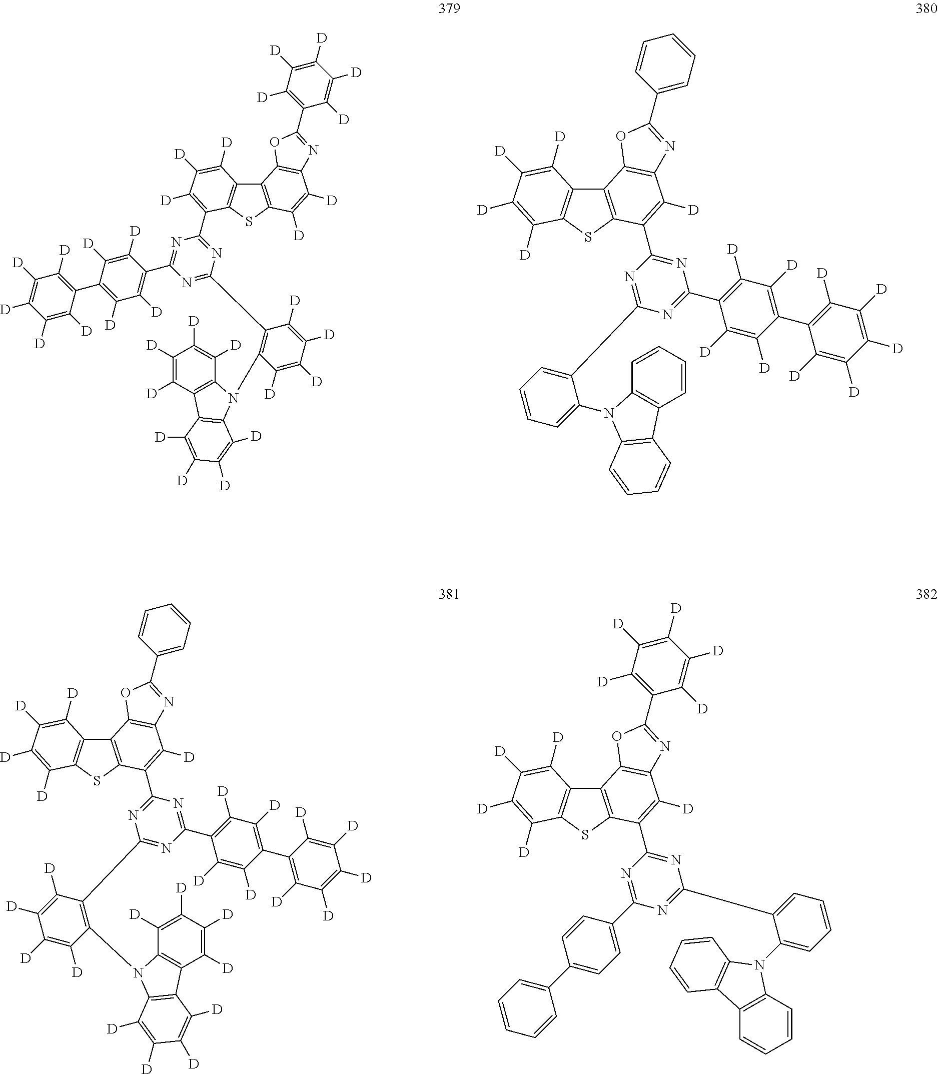

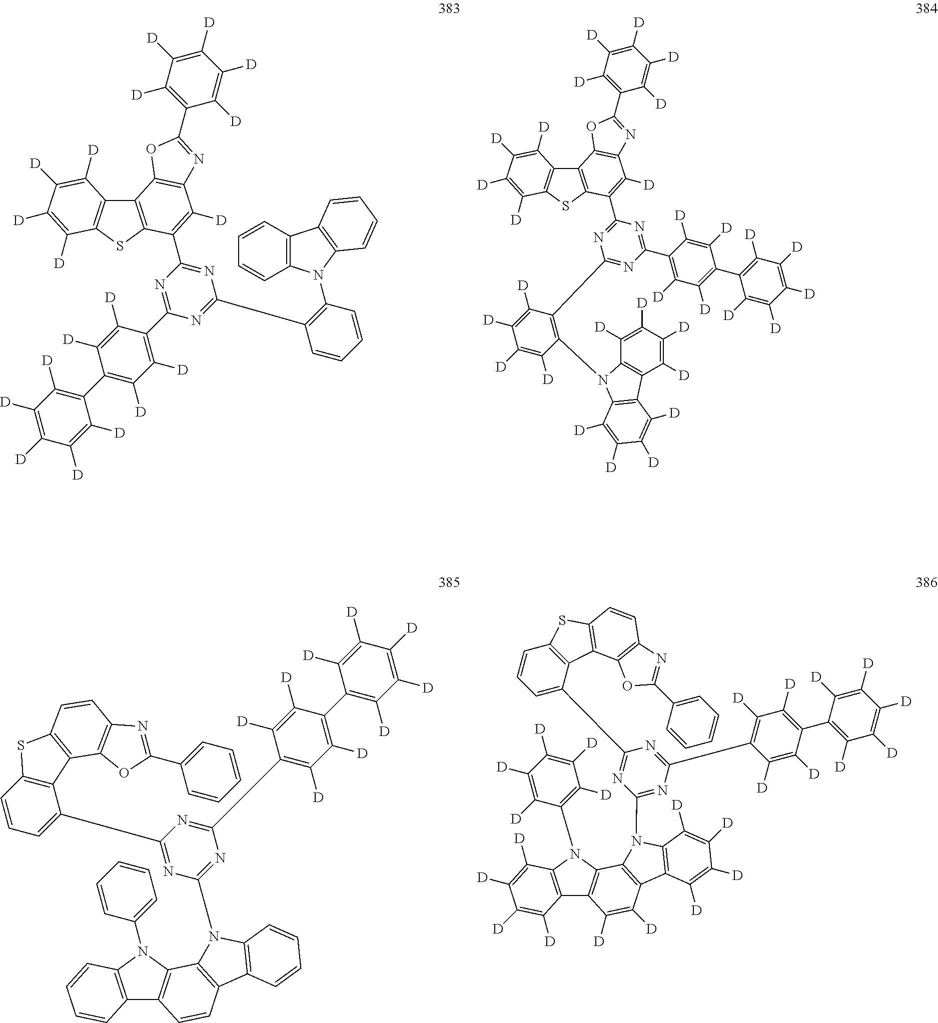

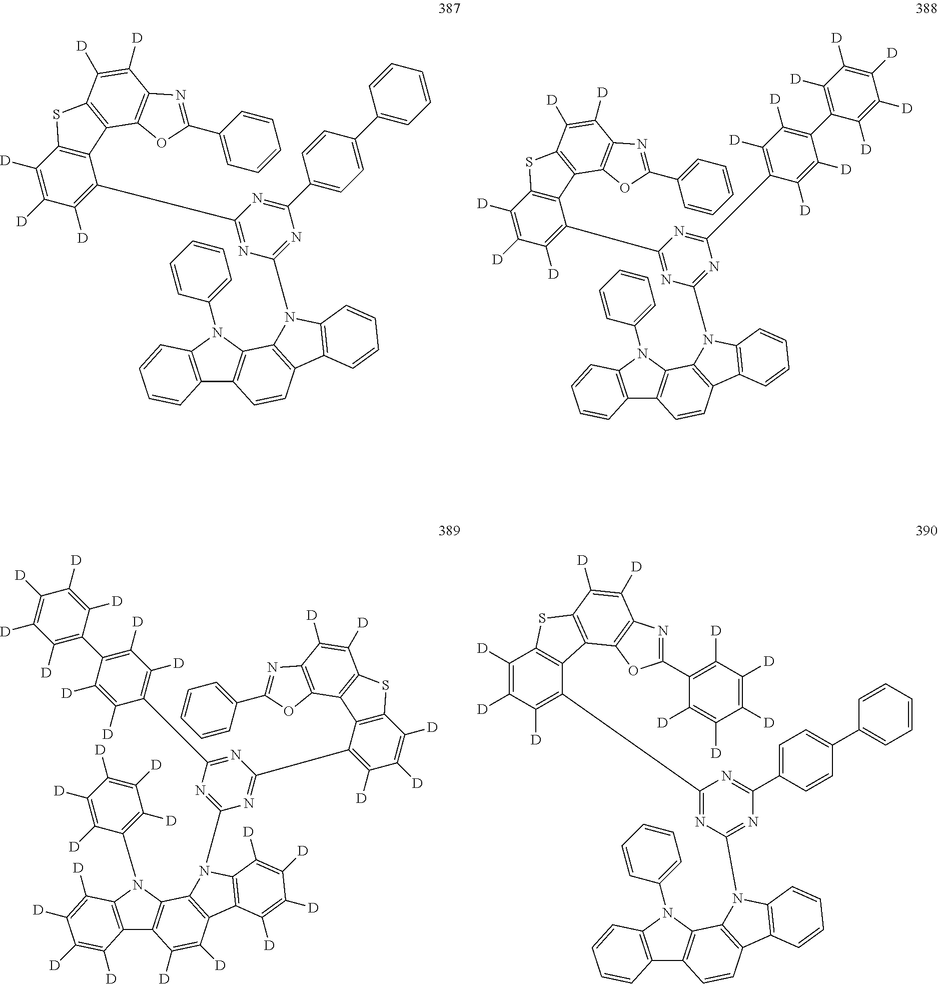

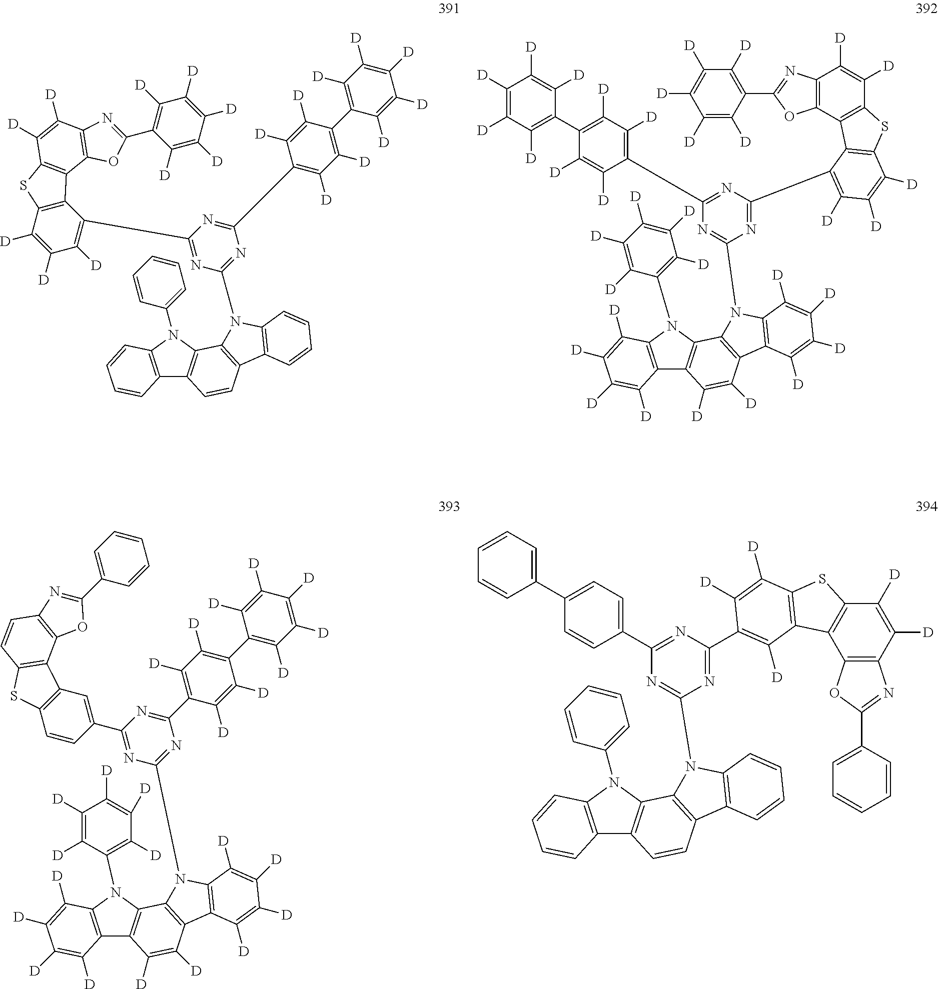

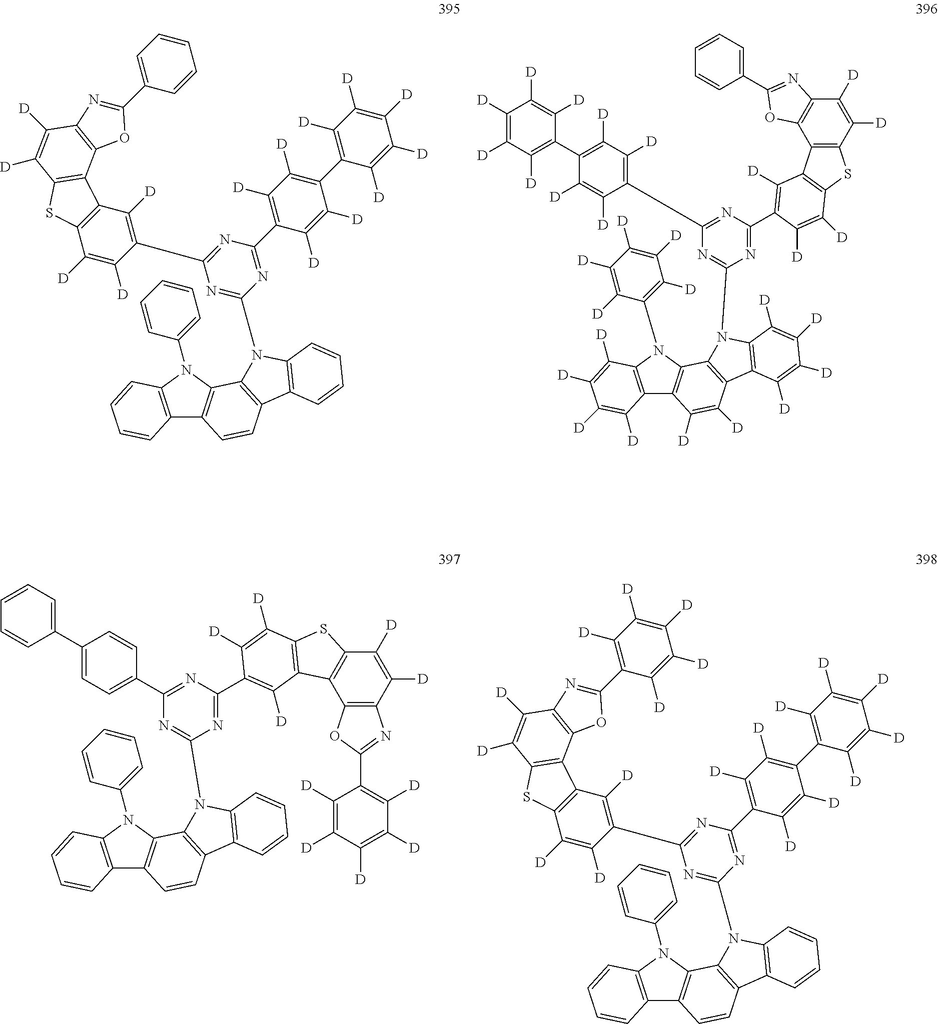

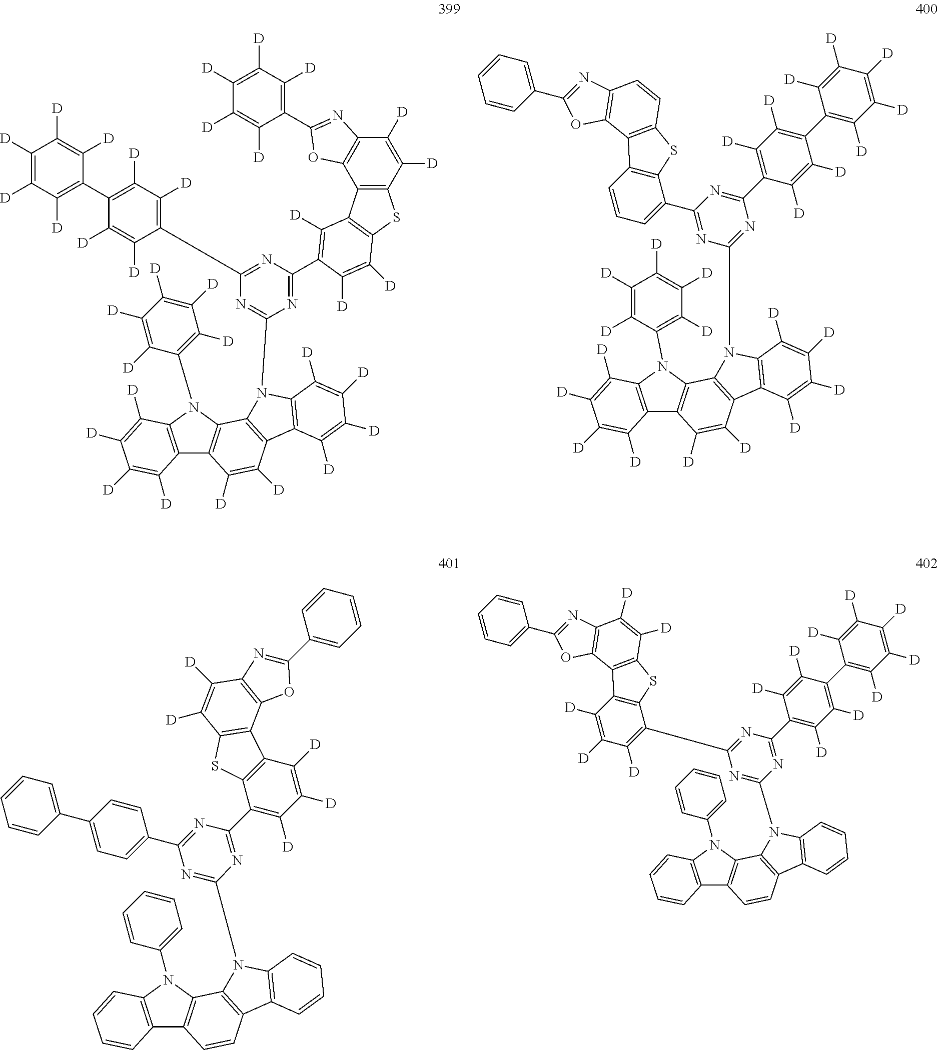

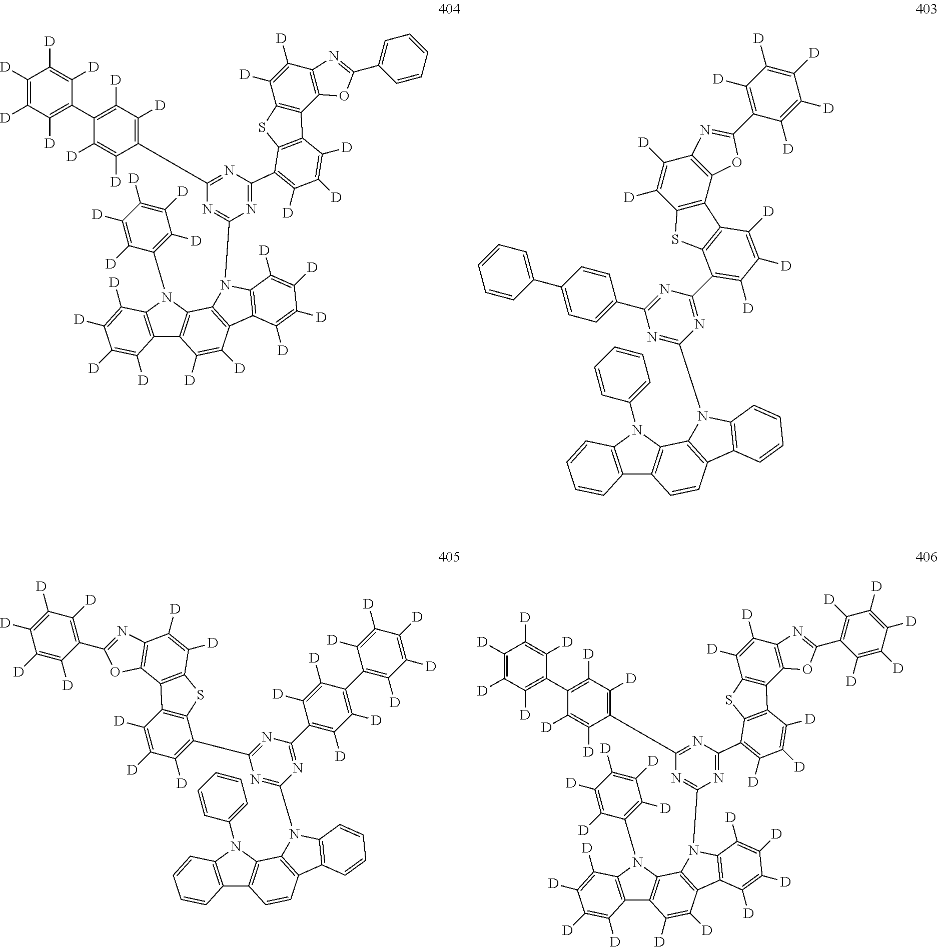

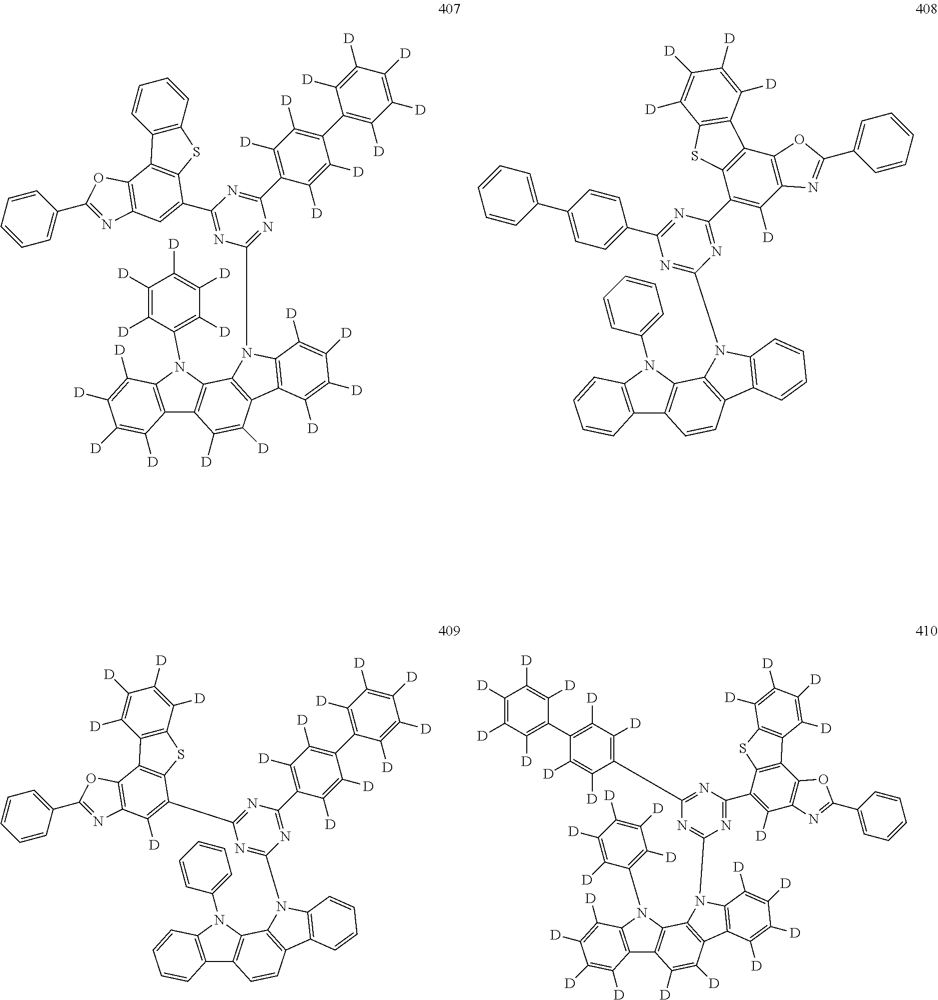

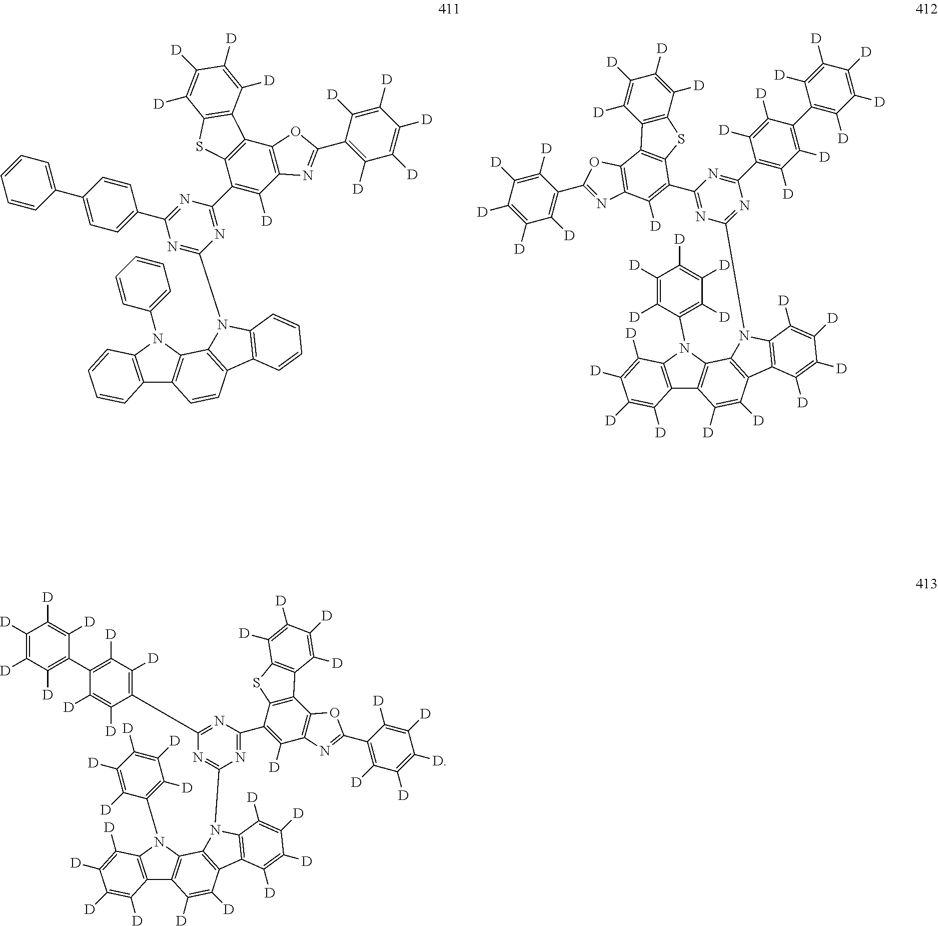

9. An organic optoelectronic device, comprising
an anode and a cathode facing each other, and
at least one organic layer between the anode and the cathode,
wherein the organic layer comprises the compound for the organic optoelectronic device as claimed in claim 1.

10. The organic optoelectronic device as claimed in claim 9, wherein
the organic layer comprises a light emitting layer, and
the light emitting layer comprises the compound for the organic optoelectronic device.

11. A display device comprising the organic optoelectronic device as claimed in claim 9.
